SEQUENCE LISTING (33 sequences)

SEQ ID NO: 1 - protein, 4 aa, Artificial Sequence
  Synthetic
GVQW

SEQ ID NO: 2 - protein, 7 aa, Artificial Sequence
  Amino acid
GVQWRDL

SEQ ID NO: 3 - protein, 9 aa, Artificial Sequence
  Amino acid
QAGVQWRDL

SEQ ID NO: 4 - protein, 10 aa, Artificial Sequence
  Amino acid
AQAGVQWRDL

SEQ ID NO: 5 - DNA, 51 bp, Artificial Sequence
  Synthetic
atatggagtttcactcttgttgcccagcctggagtgcagtggcgggatctc

SEQ ID NO: 6 - DNA, 12 bp, Artificial Sequence
  Synthetic
ggagtgcagtgg

SEQ ID NO: 7 - DNA, 15 bp, Artificial Sequence
  Synthetic
ggagtgcagtggcgg

SEQ ID NO: 8 - protein, 5 aa, Artificial Sequence
  Amino acid
GVQWR

SEQ ID NO: 9 - DNA, 21 bp, Artificial Sequence
  Synthetic
ggagtgcagtggcgggatctc

SEQ ID NO: 10 - DNA, 27 bp, Artificial Sequence
  Synthetic
cagcctggagtgcagtggcgggatctc

SEQ ID NO: 11 - protein, 17 aa, Artificial Sequence
  Synthetic
IWSFTLVAQPGVQWRDL

SEQ ID NO: 12 - protein, 16 aa, Artificial Sequence
  Synthetic
YGVSLLLPSLECSGGI

SEQ ID NO: 13 - protein, 16 aa, Artificial Sequence
  Synthetic
MEFHSCCPAWSAVAGS

SEQ ID NO: 14 - protein, 17 aa, Artificial Sequence
  Synthetic
EIPPLHSRLGNKSETPY

SEQ ID NO: 15 - protein, 16 aa, Artificial Sequence
  Synthetic
RSRHCTPGWATRVKLH

SEQ ID NO: 16 - protein, 12 aa, Artificial Sequence
  Synthetic
DPATALQAGQQE

SEQ ID NO: 17 - protein, 56 aa, Artificial Sequence
  Synthetic
MWRRRFFVFERESHSVSQAGVQWRDLGSLQPPPPGFKRFSCLSLPVAGTTGARQHG

SEQ ID NO: 18 - DNA, 27 bp, Artificial Sequence
  Synthetic
aagatcgcgccattgcactccagcctg

SEQ ID NO: 19 - DNA, 21 bp, Artificial Sequence
  Synthetic
aagatcgcgccattgcactcc

SEQ ID NO: 20 - protein, 10 aa, Artificial Sequence
  Synthetic
AQAGVQWRDL

SEQ ID NO: 21 - DNA, 30 bp, Artificial Sequence
  Synthetic
aagatcgcgccattgcactccagcctgggc

SEQ ID NO: 22 - DNA, 30 bp, Artificial Sequence
  Synthetic
gcccaggctggagtgcaatggcgcgatctt

SEQ ID NO: 23 - DNA, 21 bp, Artificial Sequence
  Synthetic
gcccaggctggagtgcaatgg

SEQ ID NO: 24 - protein, 7 aa, Artificial Sequence
  Synthetic
AQAGVQW

SEQ ID NO: 25 - DNA, 12 bp, Artificial Sequence
  Synthetic
ttgaaggtgagg

SEQ ID NO: 26 - DNA, 35 bp, Artificial Sequence
  Synthetic
gagagataatgggcgatgtttctcagggctgcttc

SEQ ID NO: 27 - DNA, 35 bp, Artificial Sequence; identical to SEQ ID NO: 26
  Synthetic

SEQ ID NO: 28 - DNA, 60 bp, Artificial Sequence
  Synthetic
gggaagaagggcggcaatgagatacagctggggaagaagggcggcaatgagatacagctg

SEQ ID NO: 29 - DNA, 83 bp, Artificial Sequence
  Synthetic
catggaaataaggaattggggcacagagataagaggtttgggcacagaaataagggattggggcacagagataaggggttggg

SEQ ID NO: 30 - DNA, 61 bp, Artificial Sequence
  Synthetic
aggtagagacaaggagagaaggggttggggtacttgccctgtccctggaaaagcagagaag

SEQ ID NO: 31 - DNA, 61 bp, Artificial Sequence
  Synthetic
gggaggggcaagtatcccaaccccttctctccgtgtctctaccccttctctgcttttctga

SEQ ID NO: 32 - DNA, 18 bp, Artificial Sequence
  Synthetic
tatcaacccttaccacaa

SEQ ID NO: 33 - DNA, 65 bp, Artificial Sequence
  Synthetic
  misc_feature (20)..(39): n is a, c, g, or t
tttcctggggcaggggcaannnnnnnnnnnnnnnnnnnnccttcacccttagccgcaagtcccgc

(19) United States
(12) Patent Application Publication
(10) Pub. No.: US 2018/0057890 A1
(43) Pub. Date: Mar. 1, 2018

(54) METHODS OF DIAGNOSIS AND THERAPEUTIC TARGETING OF CLINICALLY INTRACTABLE MALIGNANT TUMORS

(71) Applicants: Llew Keltner, Portland, OR (US); Guennadi V. Glinskii, La Jolla, CA (US)

(72) Inventors: Llew Keltner, Portland, OR (US); Guennadi V. Glinskii, La Jolla, CA (US)

(21) Appl. No.: 15/600,598

(22) Filed: May 19, 2017

Related U.S. Application Data
(60) Provisional application No. 62/339,007, filed on May 19, 2016.

(51) Int. Cl. C12Q 1/68; G06F 19/20; G06F 19/24; A61K 31/513; A61K 31/7088

(52) U.S. Cl. CPC C12Q 1/6886; G06F 19/20; G06F 19/24; C12Q 2600/106; A61K 31/7088; C12Q 2600/118; C12Q 2600/158; A61K 31/513

(57) ABSTRACT

The present disclosure is directed to methodologies or technologies for generating a predictor of a disease state (e.g. cancer-therapy efficacy status, cancer therapy progress, cancer prognosis, cancer diagnosis, therapy failure, relapse, recurrence, and the like) based on genomic and proteomic signatures, gene expression, and pathways & networks activation of endogenous human stem cell-associated retroviruses (SCAR). This disclosure is also directed to methods of targeting, designing, and using treatments for clinically intractable malignant tumors.

### CROSS-REFERENCE TO RELATED APPLICATIONS

This application claims the benefit of U.S. Provisional Patent Application No. 62/339007, filed May 19, 2016, which is incorporated herein by reference in its entirety.

All of the following related applications are also incorporated by reference in their entireties: U.S. Provisional Application No. 60/875,061, filed on Dec. 15, 2006; U.S. Provisional Application No. 60/823,577, filed on Aug. 25, 2006; U.S. Provisional Application No. 60/822,705, filed on Aug. 17, 2006; and U.S. Provisional Application No. 60/787,818, filed on Mar. 31, 2006.

### STATEMENT REGARDING SEQUENCE LISTING

The sequence listing associated with this application is provided in text format in lieu of a paper copy and is hereby incorporated by reference into the specification. The name of the text file containing the sequence listing is 58716_ST25.txt. The text file is 8 KB; was created on Oct. 24, 2017; and is submitted via EFS-Web.

### SUMMARY

In an aspect, the present disclosure is directed to, among other things, novel methods and kits for diagnosing the presence of cancer within a patient, for determining whether a subject who has cancer is susceptible to different types of treatment regimens, for monitoring the treatment of cancer within a patient, and provides novel methods of delivering cancer therapies, including individualized targeted cancer therapies. The cancers to be tested, monitored and treated include, but are not limited to, prostate, breast, lung, gastric, ovarian, bladder, lymphoma, mesothelioma, brain, liver, metastases of any of the above, and hematological cancers including but not limited to ALL, AML, and CCL. Identification of patients likely to be therapy-resistant early in their treatment regimen can lead to a change in therapy in order to achieve a more successful outcome.

In an aspect, the present disclosure is directed to, among other things, a method for diagnosing cancer or predicting cancer-therapy outcome by detecting the sequences and/or expression levels of multiple markers in the same cell at the same time, in a population of cells, or in a liquid biopsy specimen and scoring their sequences and/or expression as being qualitatively distinct or quantitatively different (above or below) in regard to a certain threshold, wherein the markers are from a particular pathway related to cancer, with the score being indicative of a cancer diagnosis or a prognosis for cancer-therapy failure. This method can be used to diagnose cancer or predict cancer-therapy outcomes for a variety of cancers. In an embodiment, the method includes determining whether an individual is experiencing SCAR's networks activation by using genetic signature information and protein signature information

In an aspect, the present disclosure is directed to, among other things, novel methods of diagnosis and therapeutic targeting of clinically intractable malignant tumors based on identification and monitoring of genomic and proteomic signatures of endogenous human Stem Cell-Associated Retroviruses (SCAR), including early detection of cancer precursor lesions. The markers can come from any pathway involved in the regulation of cancer, including specifically the SCAR's pathway and the “stemness” pathway(s). The markers can be mRNA, RNA, DNA, protein, or peptide. In an aspect, the present disclosure is directed to, among other things, novel methods of designing and using treatments for clinically intractable malignant tumors based on genomic and proteomic signatures of endogenous human stem cell-associated retroviruses (SCAR). Non-limiting examples of technologies and methodologies for detection of nucleic acids, DNA, RNA, etc., with single base mismatch specificity include those described in J.S. Gootenberg et al., “Nucleic acid detection with CRISPR-Cas13a/C2c2,” Science, doi:10.1126/science.aam9321, 2017; which is incorporated herein by reference in its entirety.

In an aspect, the present disclosure is directed to, among other things, methods and kits for diagnosing the presence of cancer within a patient, for determining whether a subject who has cancer is susceptible to different types of treatment regimens, for monitoring the treatment of cancer within a patient, and provides novel methods of delivering cancer therapies, including individualized targeted cancer therapies. The cancers to be tested, monitored and treated include, but are not limited to, prostate, breast, lung, gastric, ovarian, bladder, lymphoma, mesothelioma, brain, liver, metastases of any of the above, and hematological cancers including but not limited to ALL, AML, and CCL.. In total, the potential practical utilities of the methods have been demonstrated for 29 distinct types of human cancer.

In an embodiment, a method includes concurrently or sequentially detecting a sequence of multiple markers, the expression levels of multiple markers in the same cell at the same time, in a population of cells, or in a liquid biopsy specimen, and scoring their sequence and/or expression as being aberrant, wherein the markers are from a particular pathway related to cancer, with the score being indicative of a cancer diagnosis or a prognosis for a likelihood of cancer-therapy failure. This method can be used to diagnose cancer or predict cancer-therapy outcomes for a variety of cancers. The simultaneous co-expression of at least one, but preferably two or more markers in the same cell, population of cells, or a liquid biopsy specimen from a subject is a diagnostic for cancer and a predictor for the subject to be resistant to standard cancer therapy. The markers can come from any pathway involved in the regulation of cancer, including specifically the SCAR's pathway, PcG pathway and the “stemness” pathway(s). The markers can be mRNA, RNA, DNA, protein, or peptide.

In an aspect, the present disclosure is directed to, among other things, a novel finding that the expression of multiple markers from the SCAR's pathway above a threshold level in the same cell at the same time, wherein the markers are found within pathways related to cancer, can be used as an assay to diagnose cancer and to predict whether a patient already diagnosed with cancer will be therapy-responsive or therapy-resistant. An element of the assay is that at least one, but preferably two or more markers are detected concurrently within the same cell, population of cells, or in a liquid biopsy specimen. Marker detection can be made through a variety of detection means, including next generation sequencing and bar-coding through immunofluorescence. The markers detected can be a variety of products, including mRNA, RNA, DNA, protein, and peptide. For mRNA, RNA, and DNA based markers, next generation sequencing and/or PCR can be used as a detection means. Additionally, nucleic acid sequence, protein sequence, protein products or gene copy number can be identified through detection means known in the art. The markers detected can be from a variety of pathways related to cancer. Suitable pathways for markers include any pathways related to oncogenesis and metastasis, and more specifically include the SCAR's pathway, Polycomb group (PcG) chromatin silencing pathway and the “stemness” pathway(s).

In an aspect, the present disclosure is directed to, among other things, a method for diagnosing cancer or predicting cancer-therapy outcome in a biological subject.

In an embodiment, the method includes obtaining a biological sample (e.g., tissue, a cell, a specimen of bodily fluid, biological fluid, biomarker composition, and the like) from the subject.

In an embodiment, the method includes selecting a marker from a pathway related to cancer,

In an embodiment, the method includes screening for simultaneous aberrant sequences and/or expression level of at least one but preferably, two or more markers.

In an embodiment, the method includes scoring their sequence(s) as being aberrant when the quality of the sequence (the defined sequence of the positions of the bases within an entire sequence or its fragment) is distinct compared with the reference sequences, and

In an embodiment, the method includes scoring their expression level as being aberrant when the expression level detected is above a certain threshold.

In an embodiment, the method includes the presence of an aberrant sequence and/or an aberrant expression level of at least one but preferably, two or more such markers is indicative of a cancer diagnosis or a prognosis for cancer-therapy failure in the subject.

In an embodiment, an aberrant sequence and/or co-expression level of the markers can be indicative of the presence of cancer in the subject, or predictive of cancer-therapy failure in the subject. The markers can be selected from any suitable cancer pathway, including in preferred embodiments markers from the SCAR's or “stemness” pathway (s). For aberrant sequences detection, these markers can be genes selected from the group consisting of ELF3; PCDH15; MALAT1; PTPN11; RB1; CHST6; NF1; VEZF1; TP53; SMAD4; KEAP1; STK11; PRX; ZNF28; IDH1; FEZ2; DPPA2; LPHN3; KIAA1244; EPHA7; EGFR; TLR4; DAB2IP; NOTCH1; GLUD2; DMD; KDM6A; KRAS; CDKN2A; DNMT3A; FLT3; NFE2L2; NPM1; MIR142; FOXL2; H3F3A; H3F3B; KMT2D ; RNF43 ; TERT; ERBB2; PLCG1. For aberrant expression detection, these markers can be genes selected from the group consisting of PLCXD1, HKR1, ZNF283, ADA, AMACR+p63, ANK3, BCL2L1, BIRC5, BMI-1, BUB1, CCNB1, CCND1, CES1, CHAF1A, CRIP1, CRYAB, ESM1, EZH2, FGFR2, FOS, Gbx2, HCFC1, IER3, ITPR1, JUNB, KLF6, KI67, KNTC2, MGC5466, Phc1, RNF2, Suz12, TCF2, TRAP100, USP22, Wnt5A and ZFP36. In preferred embodiments, the markers are selected from the group consisting of regulatory and down-stream genetic elements of the SCAR's pathway(s), transcription factors, and methylation patterns. In one preferred embodiment, the aberrant sequence(s) being detected and in another preferred embodiment the aberrant co-expression level being detected is of regulatory and down-stream genetic elements of the SCAR's pathway(s), transcription factors, and methylation patterns. The markers being detected are in the form of either mRNA, RNA, DNA, protein, or peptide.

In an embodiment, the aberrant expression level of at least one but preferably, two or more markers can be detected by any detection means known in the art, including, but not limited to, subjecting the cells to an analysis selected from the group consisting of next generation sequencing, multicolor quantitative immunofluorescence co-localization analysis, fluorescence in situ hybridization, and quantitative RT-PCR analysis.

In an aspect, the present disclosure is directed to, among other things, a method for concurrently detecting an aberrant sequence(s) and/or co-expression level of at least one but preferably, two or more markers in a single cell, population of cells, or liquid biopsy samples. In an embodiment, obtaining a sample of tissue, a cell, or a specimen of bodily fluid. In an embodiment, selecting a marker defined by a pathway. In an embodiment, screening for a simultaneous aberrant sequences and/or expression level of at least one but preferably, two or more markers. In an embodiment, scoring their sequence(s) as being aberrant when the quality of the sequence (the sequence of the positions of the bases within an entire sequence or its fragment) is distinct compared with the reference sequences. In an embodiment, scoring their expression level as being aberrant when the expression level detected is above a certain threshold.

In an aspect, the present disclosure is directed to, among other things, a method for detecting at least one of an aberrant sequence(s) and/or co-expression level of at least one but preferably, two or more markers in a single cell, population of cells, or liquid biopsy samples. In an embodiment, obtaining a sample of tissue, a cell, or a specimen of bodily fluid. In an embodiment, selecting a marker defined by a pathway. In an embodiment, screening for a simultaneous aberrant sequences and/or expression level of at least one but preferably, two or more markers. In an embodiment, scoring their sequence(s) as being aberrant when the quality of the sequence (the sequence of the positions of the bases within an entire sequence or its fragment) is distinct compared with the reference sequences. In an embodiment, scoring their expression level as being aberrant when the expression level detected is above a certain threshold.

In an aspect, the present disclosure is directed to, among other things, kits useful in detecting the concurrently aberrant sequences or co-expression levels of two or more markers in a single cell, population of cells, or liquid biopsy samples. In an aspect, the present disclosure is directed to, among other things, kits useful in detecting at least one of an aberrant sequences or co-expression levels of two or more markers in a single cell, population of cells, or liquid biopsy samples.

In an aspect, the present disclosure is directed to, among other things, a method of targeted therapy of malignant tumors which harbor the molecular markers selected from any suitable cancer pathway, including in preferred embodiments markers from the SCAR's or “stemness” pathway(s). Therapeutic targeting of said malignant tumors is guided by the markers being detected in the form of either mRNA, RNA, DNA, protein, or peptide. In preferred embodiments, therapeutic modalities are designed toward molecular targets selected from the group consisting of regulatory SCARs loci and down-stream genetic elements of the SCAR's pathway(s).

The present disclosure details one or more methodologies or technologies for diagnosing cancer, predicting cancer-therapy outcome, determining whether a subject who has cancer is susceptible to different types of treatment regimens, monitoring the efficacy of a cancer treatment, determining, a cancer diagnosis or a prognosis for cancer-therapy failure, and the like by detecting the sequences, expression levels, gene levels, transcription levels, and the like for multiple markers.

In an embodiment, one or more methodologies or technologies for diagnosing untreatable cancer (e.g., one with activated endogenous human Stem Cell-Associated Retroviruses (SCAR) network) include one or more of detecting mutations of the sequences of 42 genes (listed in FIG. 16); analyzing transcription levels of specific SCAR sequences; analyzing levels of protein sequences; analyzing expression levels in signatures, determining gene expression levels and determining gene copy numbers of Data Set S1 (Tables 4-9), Data Set S2 (Tables 10-14), and Data Set S3 (Tables 15-17).

For example, in an embodiment, methodologies or technologies include generating a user-specific cancer therapy protocol, or a user-specific cancer diagnosis, responsive to receiving one or more inputs indicative of an aberrant sequence or an aberrant expression level associated with the expression levels of one or more locus or loci listed in Table 3.3. Non-limiting examples of genomic signature pathways, signature evaluation method, and the like can be found in U.S. Pat. Nos. 8,349,555 and 7,890,267; each of which is incorporated herein by reference in its entirety.

In an embodiment, methodologies or technologies include generating a predictor of a disease state (e.g., a cancer-therapy efficacy status, cancer therapy progress, a cancer prognosis, a cancer diagnosis, therapy failure, relapse, recurrence, and the like) responsive to receiving one or more inputs indicative of an aberrant expression level associated with the expression levels of one or more peptides listed in FIGS. 18A and 18B.

In an embodiment, methodologies or technologies include generating a predictor of a disease state (e.g., a cancer-therapy efficacy status, cancer therapy progress, a cancer prognosis, a cancer diagnosis, therapy failure, relapse, recurrence, and the like) responsive to receiving one or more inputs indicative of the SCAR's pathway activation signatures for genes listed in FIGS. 19A and 19B.

In an embodiment, methodologies or technologies include generating a SCARs activation status responsive to receiving one or more inputs indicative of an aberrant expression level associated with the expression levels of one or more locus or loci listed in FIGS. 20A-20C.

In an embodiment, methodologies or technologies include generating a predictor of a disease state (e.g., a cancer-therapy efficacy status, cancer therapy progress, a cancer prognosis, a cancer diagnosis, therapy failure, relapse, recurrence, and the like) responsive to receiving one or more inputs indicative of an aberrant expression level associated with the expression levels of one or more locus or loci listed in FIGS. 21A-21C.

In an embodiment, methodologies or technologies include generating a predictor of a disease state (e.g., a cancer-therapy efficacy status, cancer therapy progress, a cancer prognosis, a cancer diagnosis, therapy failure, relapse, recurrence, and the like) responsive to receiving one or more inputs indicative of an aberrant expression level or a gene copy number associated with the expression levels or the copy number of one or more locus or loci listed in Data Set S1 (Tables 4-9).

In an embodiment, methodologies or technologies include generating a predictor of a disease state (e.g., a cancer-therapy efficacy status, cancer therapy progress, a cancer prognosis, a cancer diagnosis, therapy failure, relapse, recurrence, and the like) responsive to receiving one or more inputs indicative of an aberrant expression level associated with the expression levels of one or more sequences listed in Data Set S2 (Tables 10-14).

In an aspect, the present disclosure is directed to, among other things, a method of identification of common peptide sequences encoded by the genomic loci derived from SCAR sequences. In an embodiment, the method includes retrieving nucleic acid sequences of the SCARs-derived genomic loci which are located at distinct genomic coordinates; and identifying all open reading frames (ORFs) within said nucleic acid sequences. In an embodiment, the method further includes identifying all peptide sequences encoded by and potentially transcribed from said nucleic acid sequences; and Identifying peptide sequences common for distinct SCAR-derived genomic loci which are located at distinct genomic coordinates.

In an embodiment, methodologies or technologies include determining SCAR's networks activation using genetic signature information and protein signature information. In an embodiment, SCAR's networks activation information is used to generate a cancer outcome prognosis. For example, activated SCAR's networks is indicative of a poor cancer therapy outcome or a poor prognosis.

In an embodiment, methodologies or technologies include generating a cancer related outcome based on one more inputs indicative of an aberrant sequence and one more inputs indicative of an expression level of SCARs networks markers

Non-limiting examples of SCAR's networks include a genome-wide compendium of: i) transcriptionally-active SCAR's loci defined based on detection of the expression of corresponding RNA molecules; and ii) expression signatures of down-stream SCARs-regulated coding genes, including protein-coding genes, genes encoding non-coding RNA molecules, micro-RNAs, and other regulatory & structural molecules affected by SCARs activity.

Non-limiting examples of a SCAR pathway include a sub-set of SCAR's loci that are transcriptionally active in specific cells and/or specific biological samples, including single cells as well as populations of cells.

SCAR's pathways: a sub-set of genomic loci defined by the genome-wide SCAR's networks analyses in specific cells and/or specific biological samples, including single cells as well as populations of cells.

Non-limiting example of signatures include 74-gene signature (referring to table S4 for example), 55-gene signature (referring to table S4 for example), the SCAR's pathway signatures defined by the single cell analysis of human oocytes in which expression changes of these genes appear associated with activated transcription of HERV-H-derived retroviral sequences. The gene symbols are listed in the first column. These are coding genes expression of which is altered in a specific manner (up- and down-regulated) using shRNA-interference protocol targeting HERV-H-encoded regulatory transcripts (the log-transformed fold expression changes are listed in the second column). Expression changes of these genes in human oocytes (the log-transformed fold-expression changes are listed in the third column) are consistent with the HERV-H-pathway activation (r=−0.74043), that is genes expression of which is up-regulated following the shHERVH interference appear down-regulated in oocytes; conversely, genes expression of which is down-regulated following the shHERVH interference appear up-regulated in oocytes. The utility of these signatures have been demonstrated by the analyses of samples of normal and pathological human prostates, including prostate cancer samples and prostatic intraepithelial neoplasia samples (FIGS. 1C & 2D). The fold expression changes of each of the individual gene listed in the Table S4 would be determined using the technologies and methods known to the individuals skilled in the art. The values for corresponding genes will be listed in the order defined in the Table S4 as it is shown for the oocyte's values listed in the third column. Next, the correlation coefficient is computed for the values listed in the second and the third columns. The negative values of the correlation coefficient should be interpreted as the indication of the SCAR's pathway activation. The positive values of the correlation coefficient would indicate no evidence of SCAR's pathway activation.

In an embodiment, genetic signatures and protein signatures are used as predictors of a disease state independently. In an embodiment, some specific gene/protein targets listed in current signatures are likely relevant to cancer. In an embodiment, some specific gene/protein targets listed in current signatures are utilized them to detect the SCAR's pathways & networks activation.

### BRIEF DESCRIPTION OF THE FIGURES

FIGS. 1A-1K collectively illustrate distinct expression patterns of HERVH-regulated genes in euploid and aneuploid human embryos at 1-cell versus 8-cell stages (FIGS. 1A-1D), developmentally viable versus non-viable zygotes (A, FIG. 1D), and in vivo matured human oocytes (FIGS. 1E-1H).

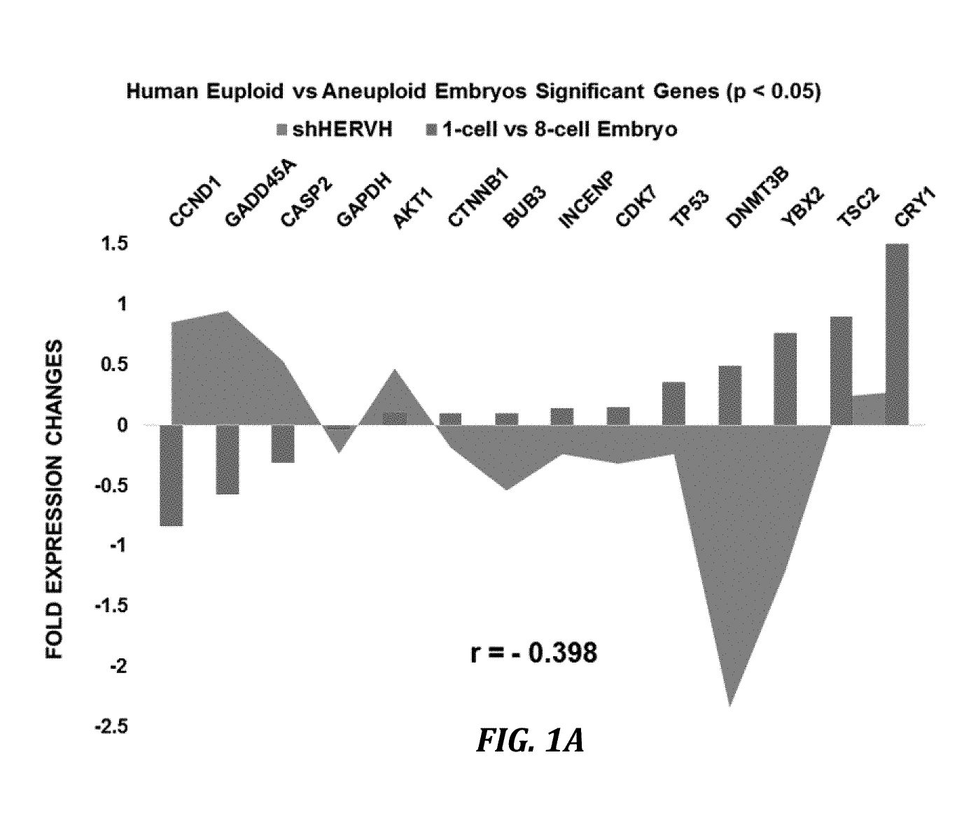

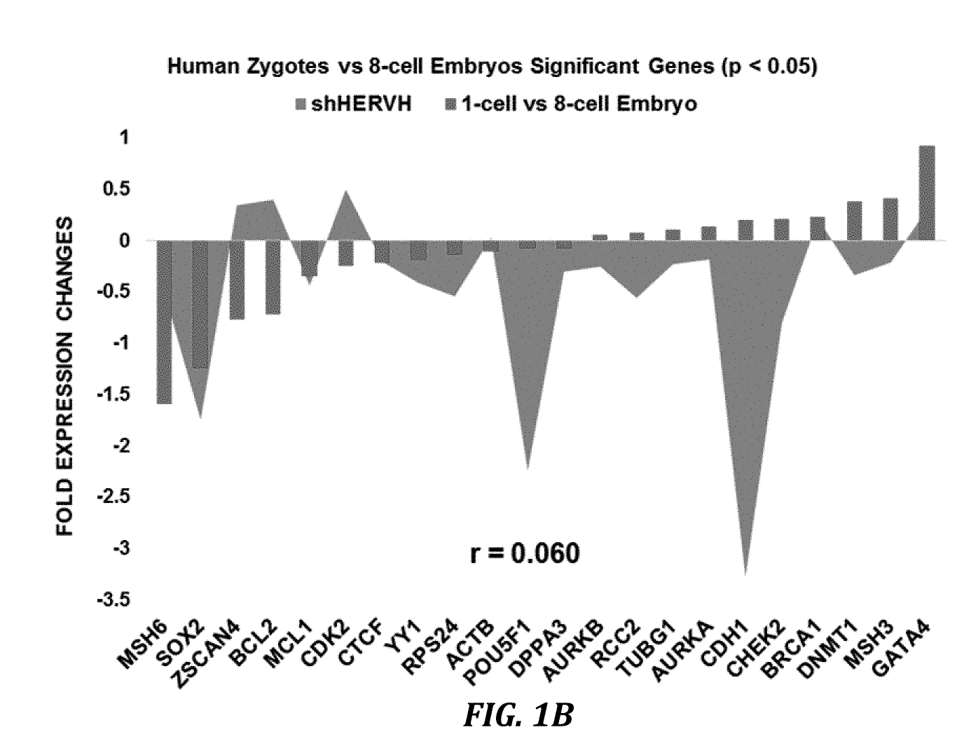

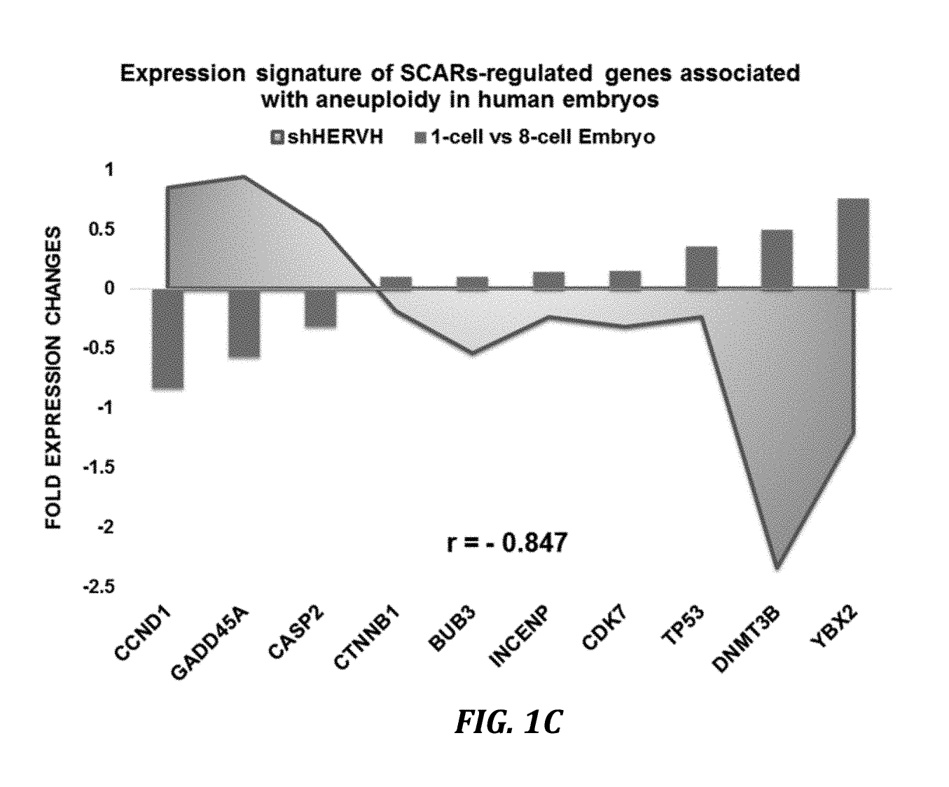

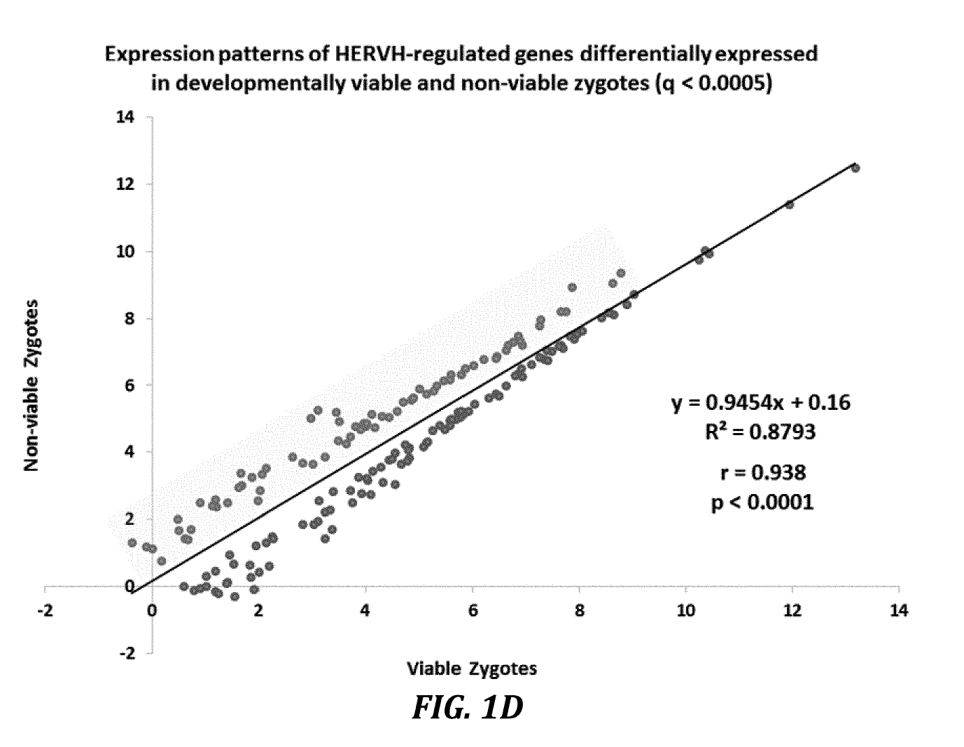

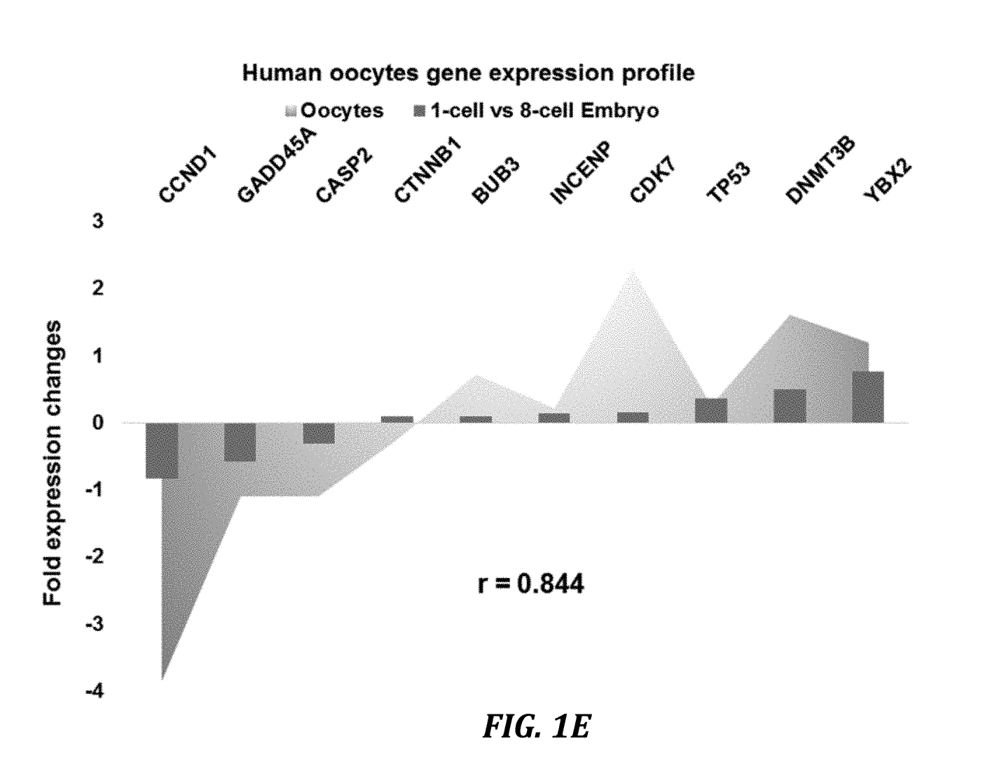

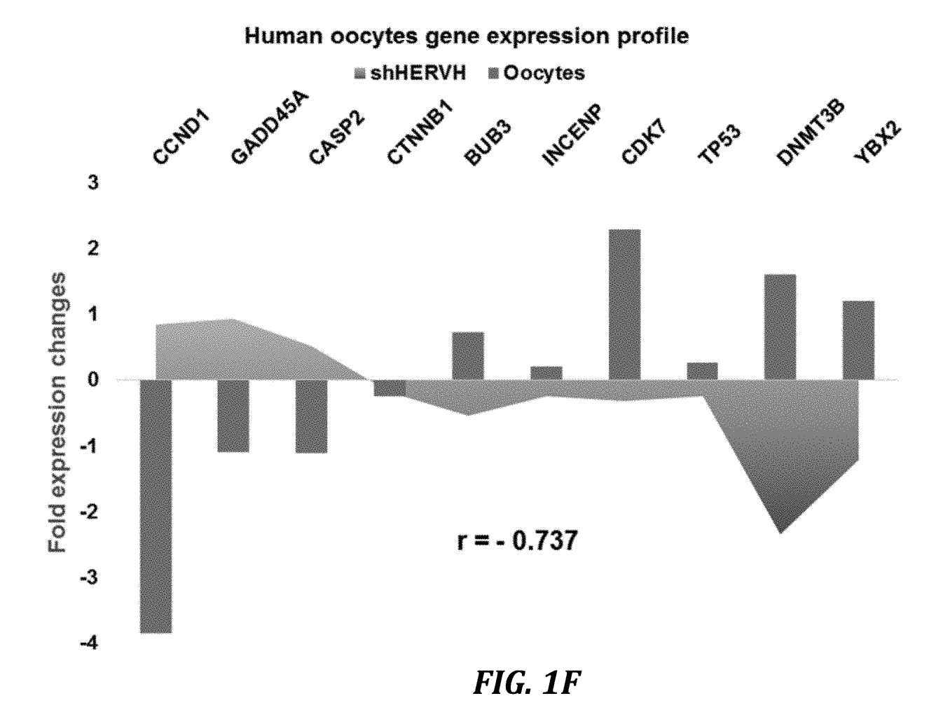

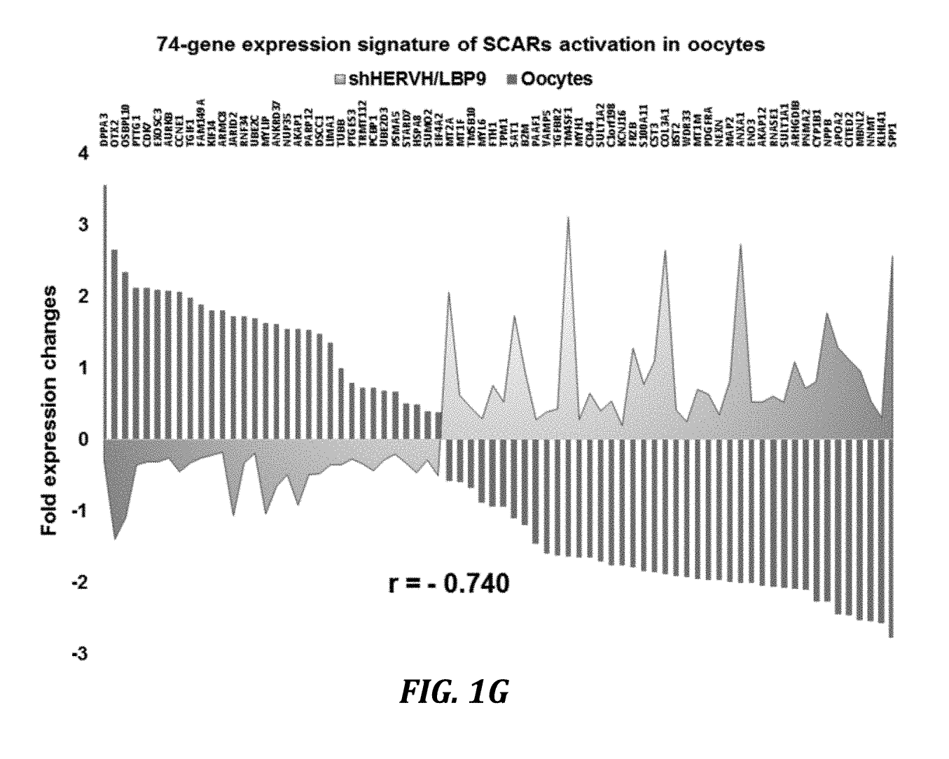

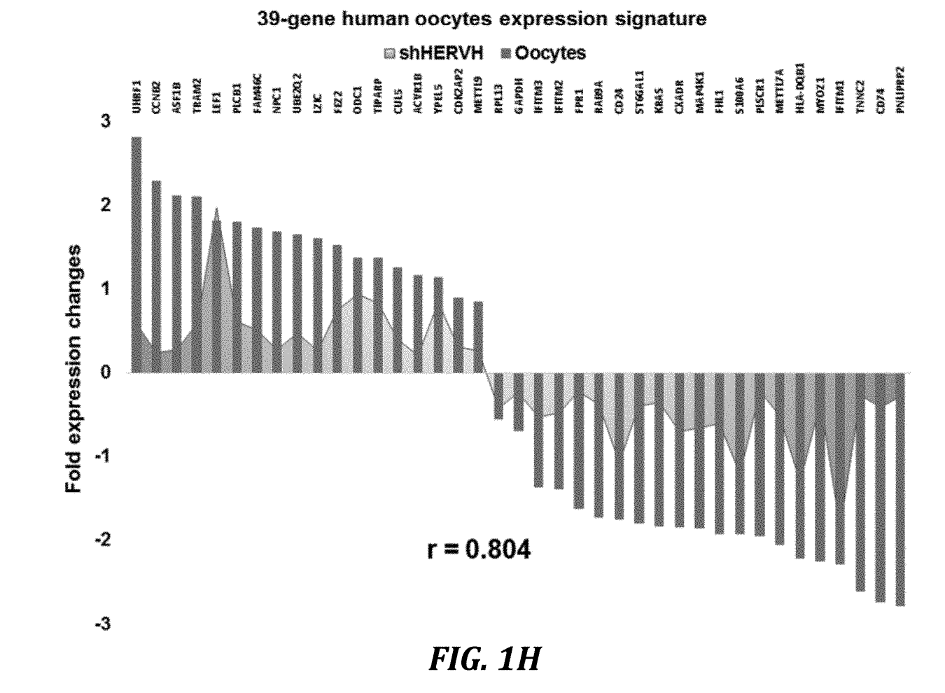

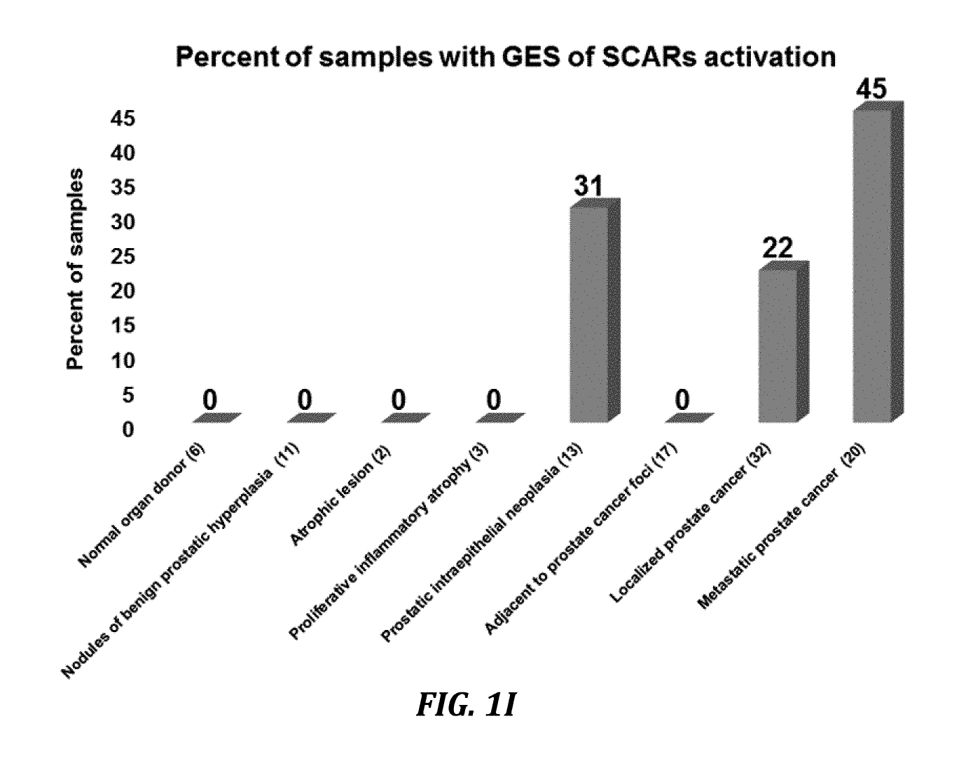

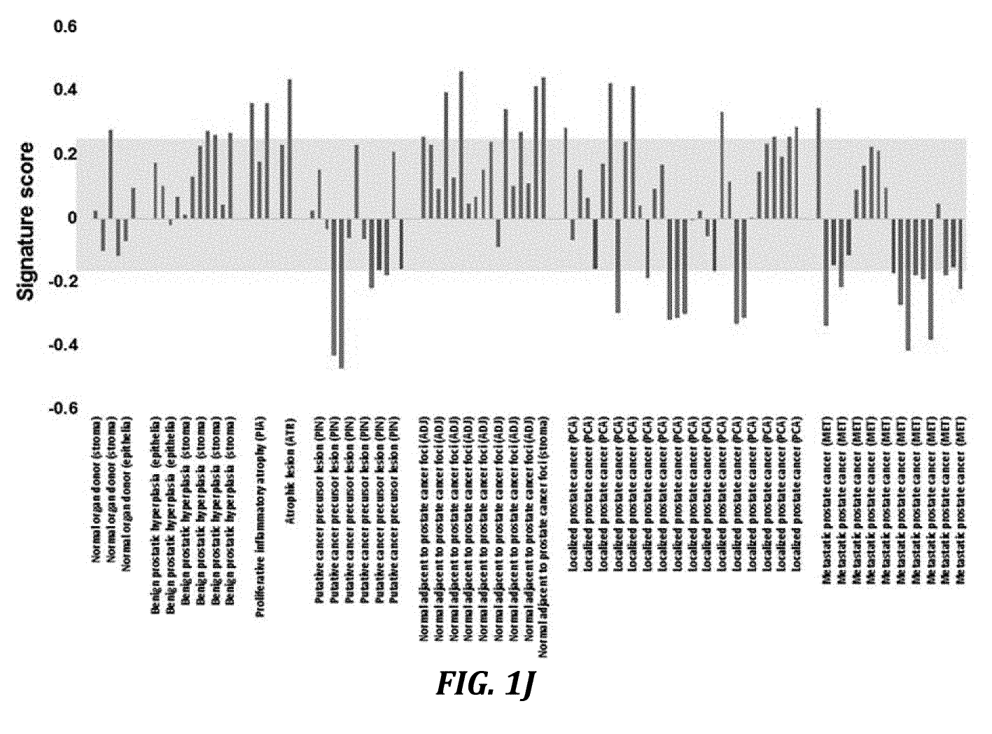

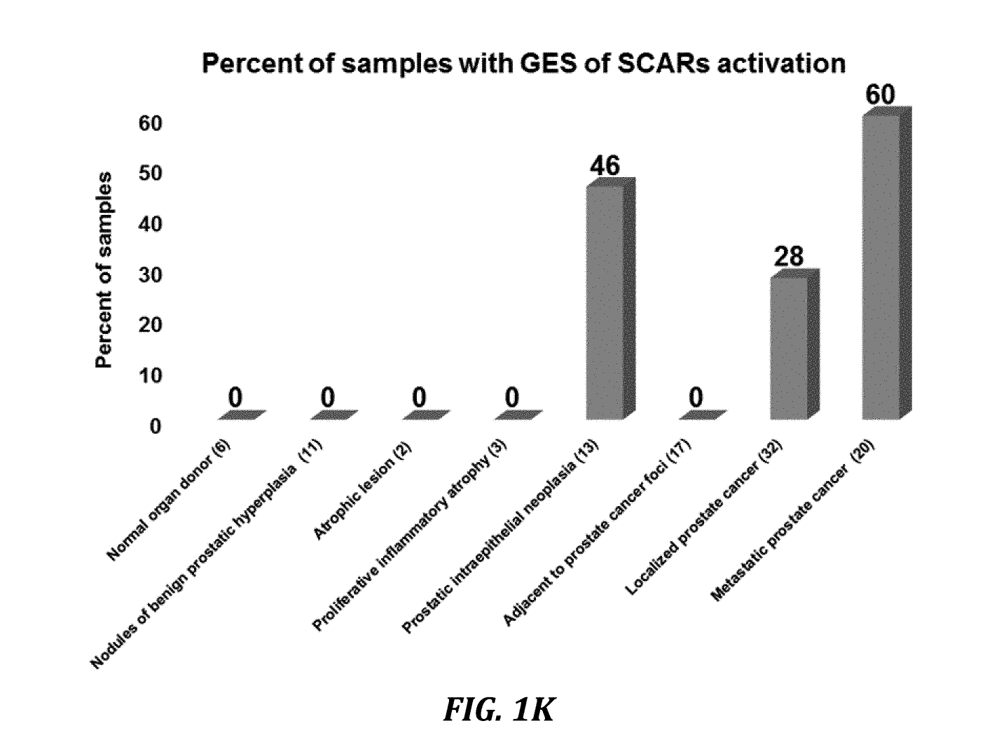

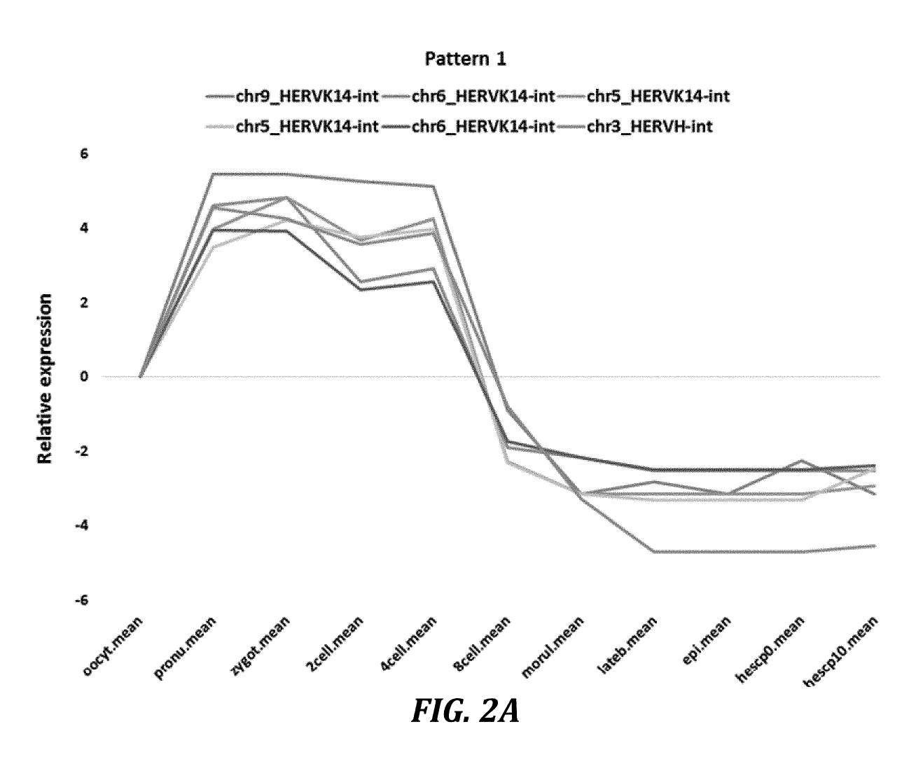

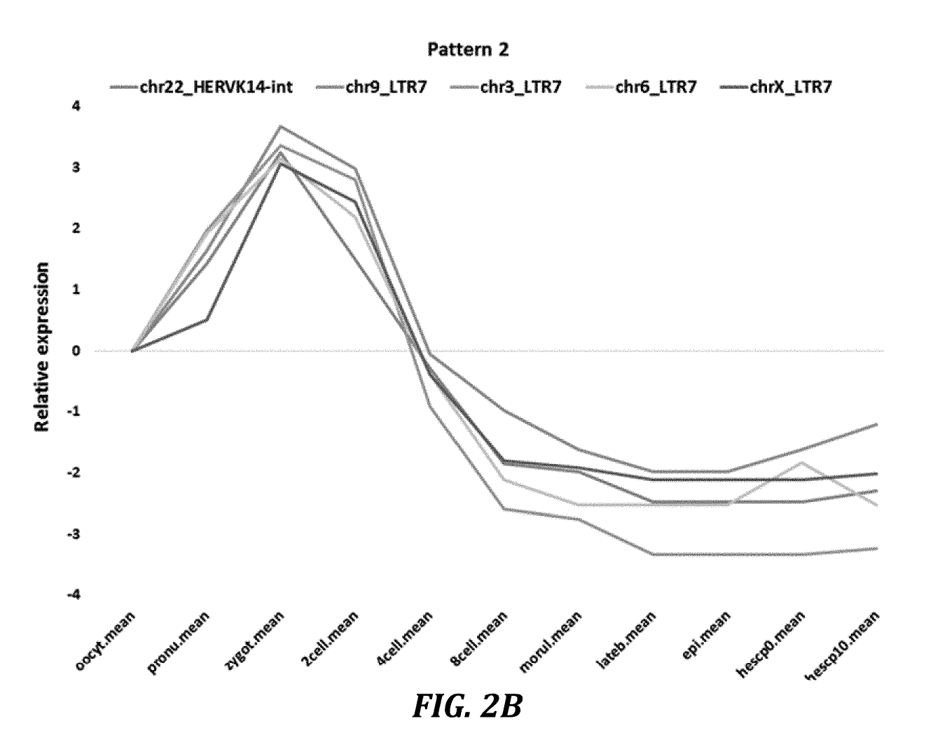

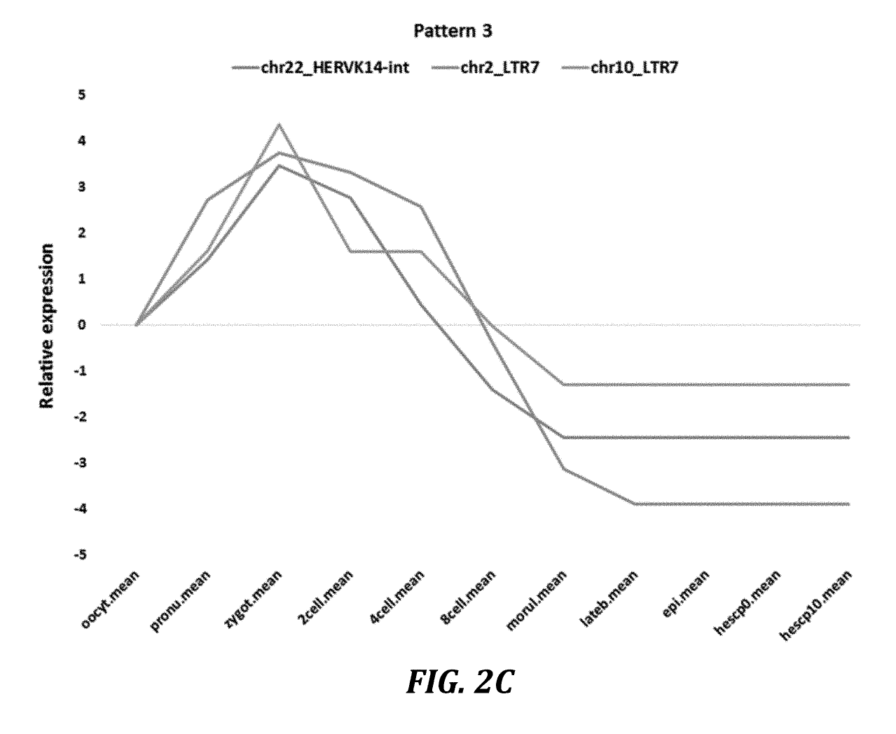

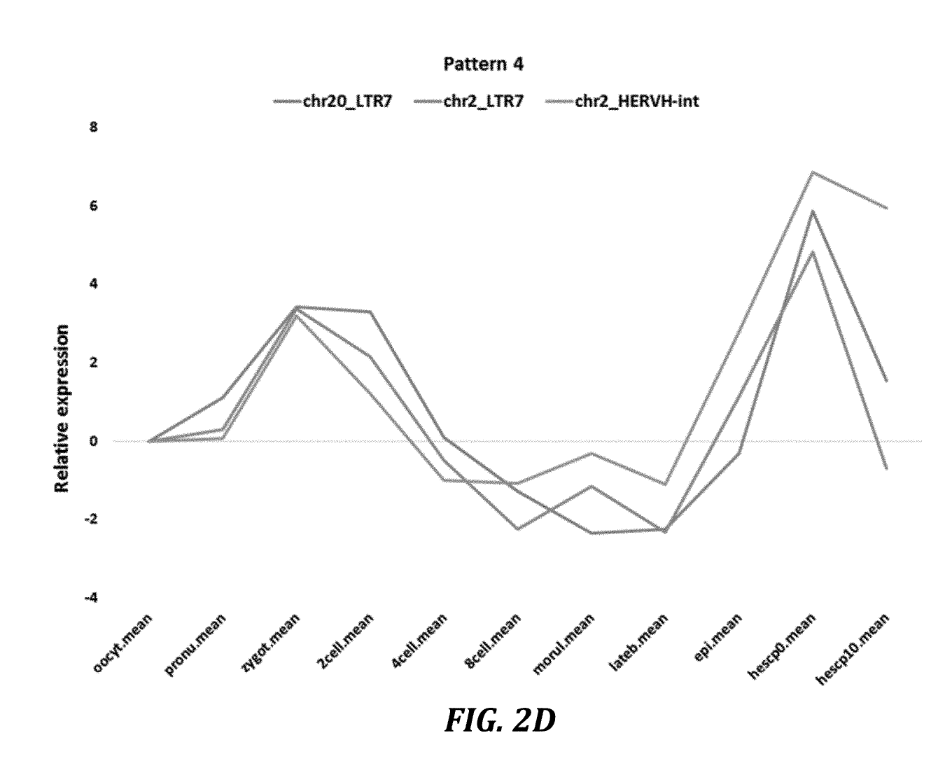

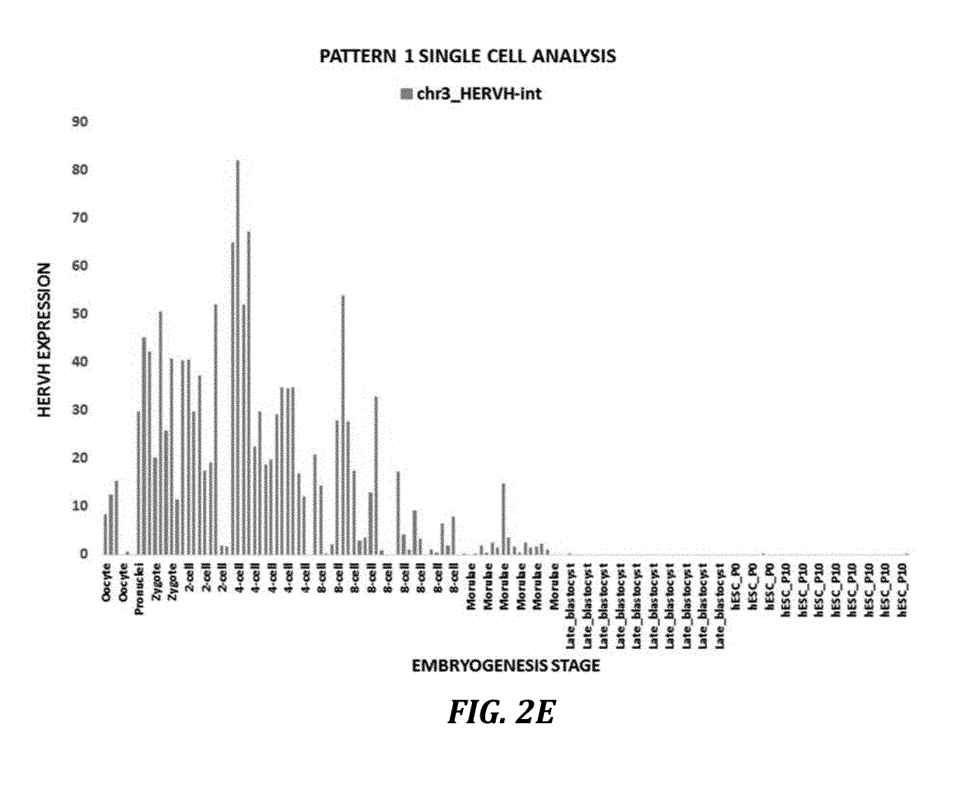

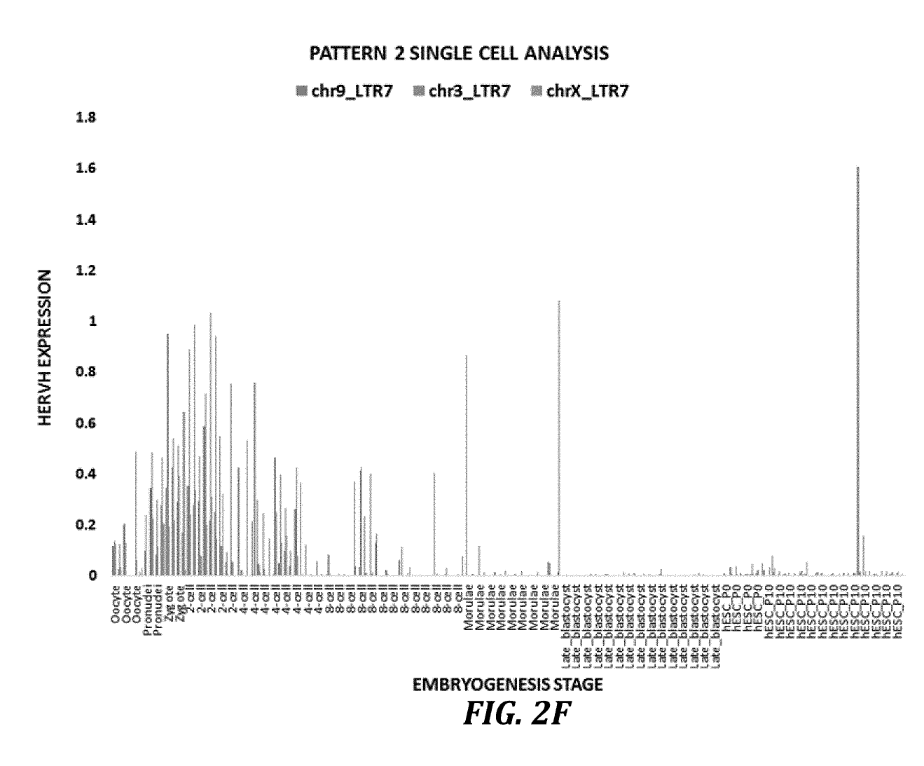

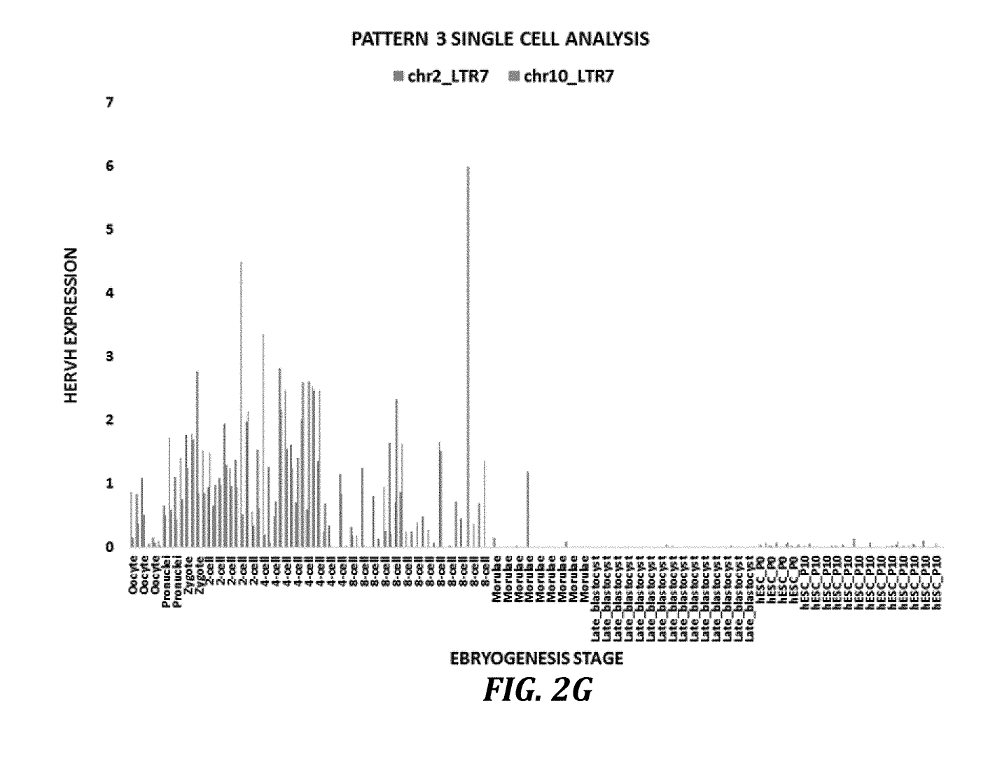

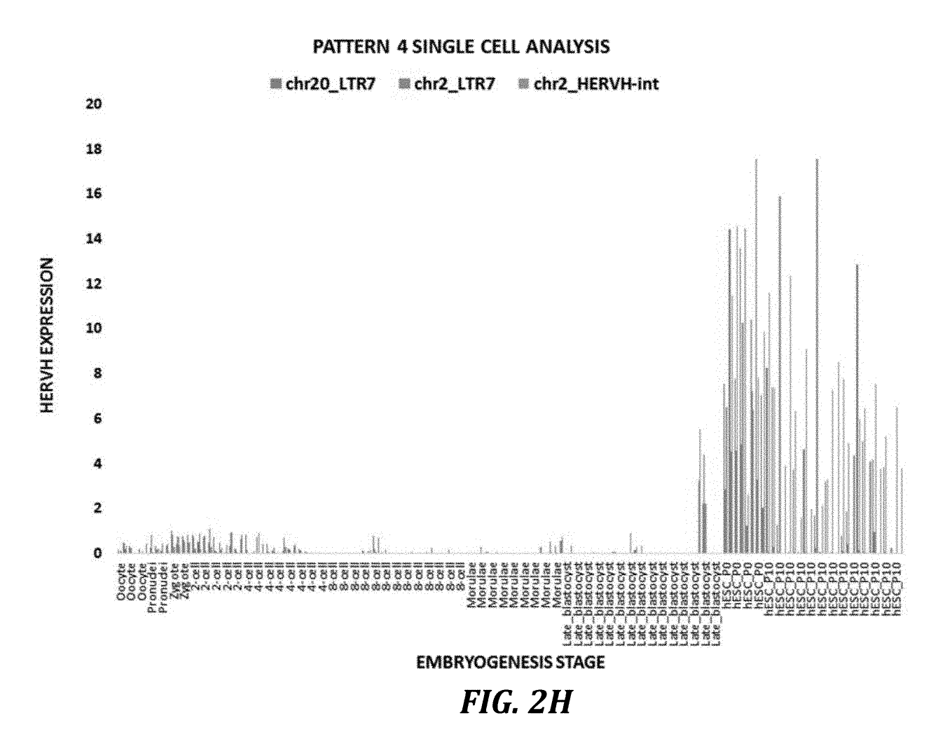

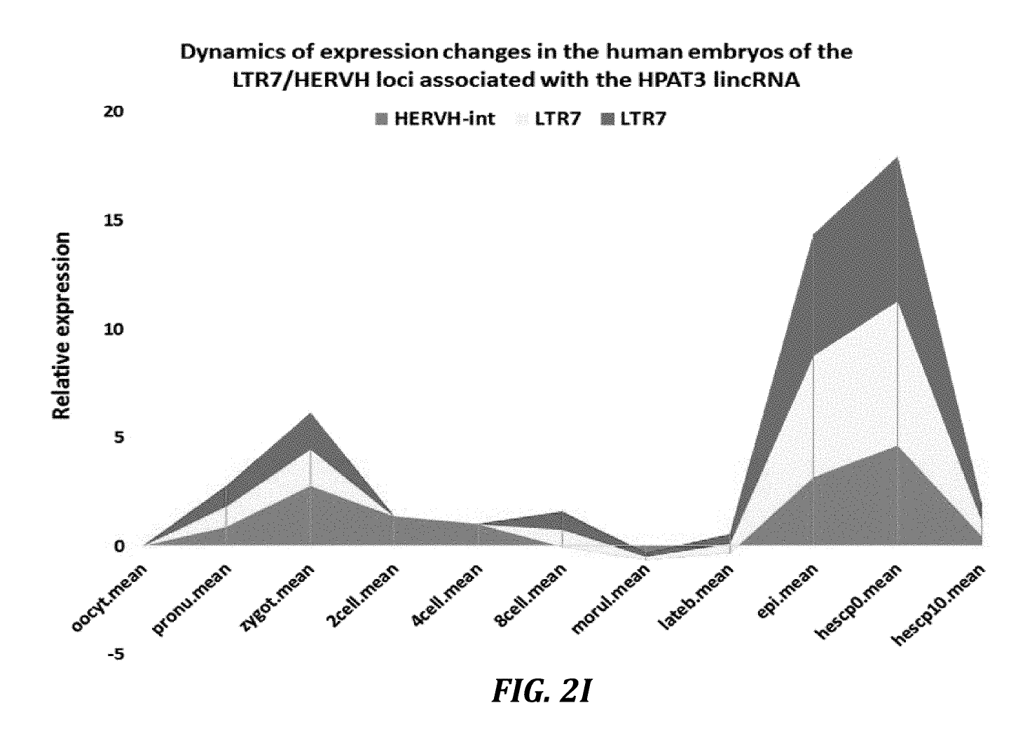

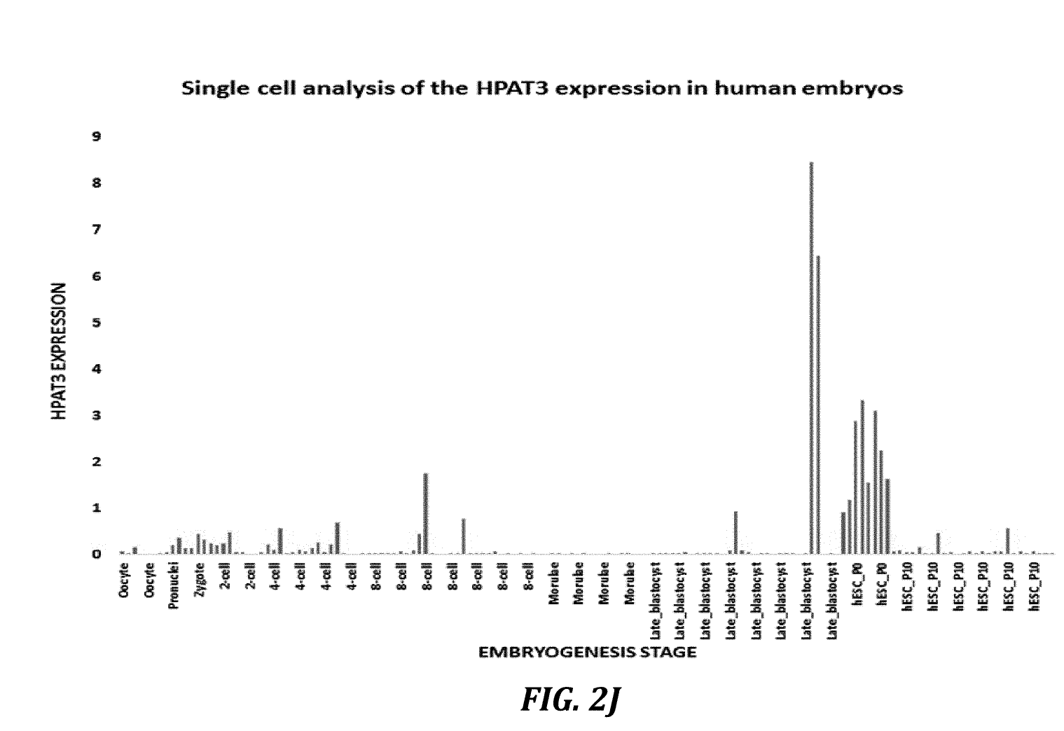

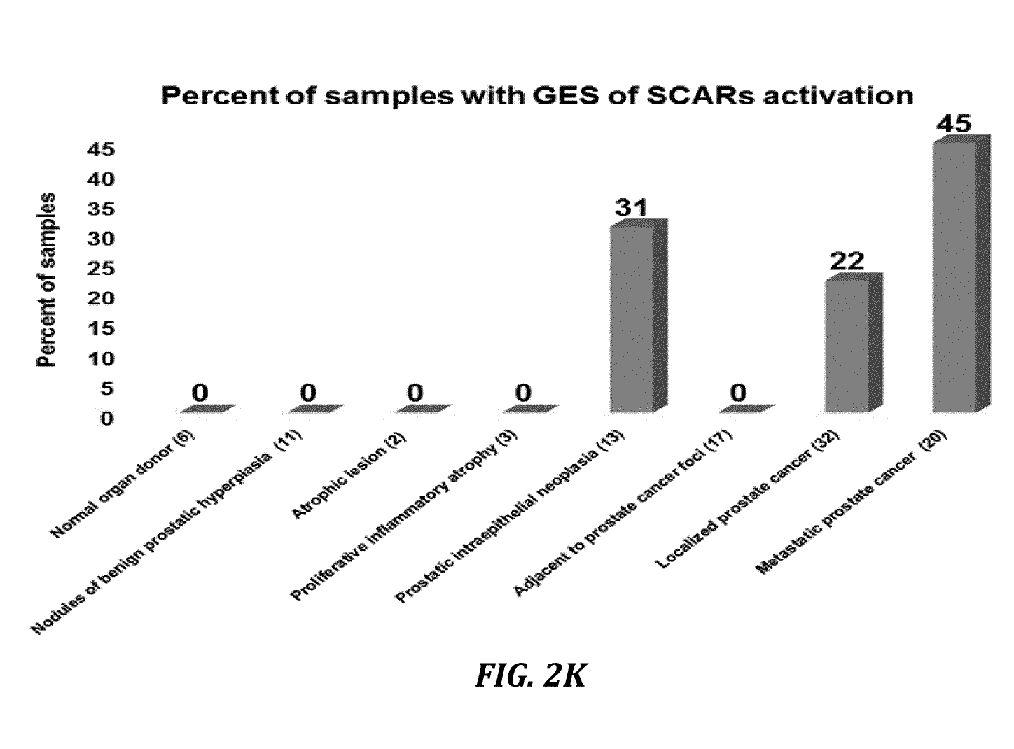

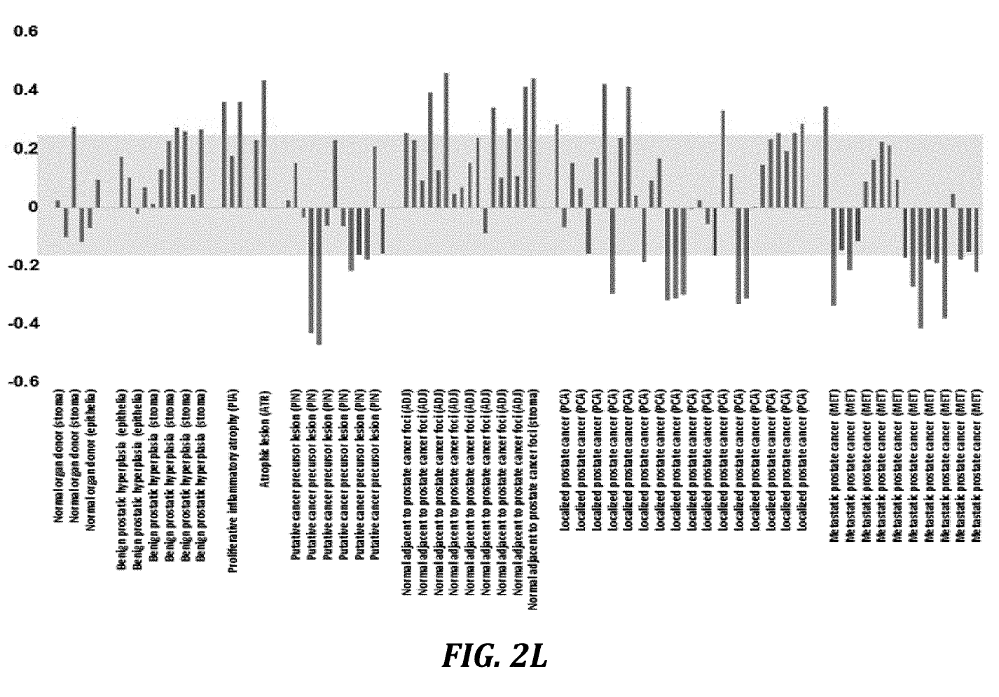

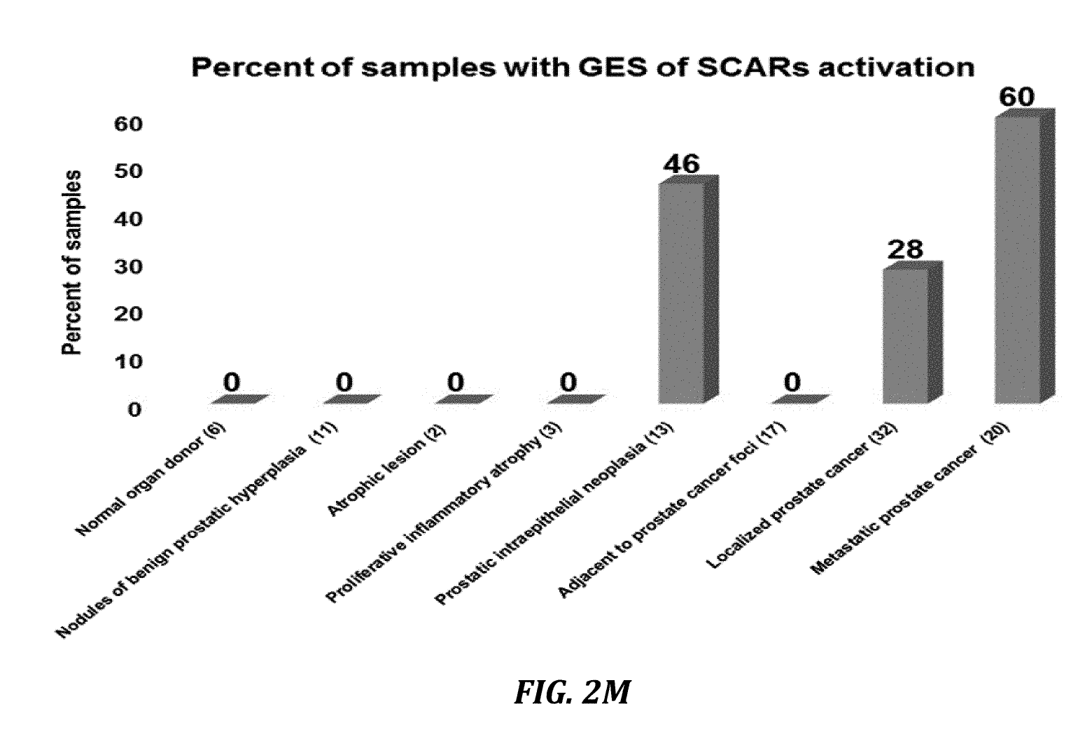

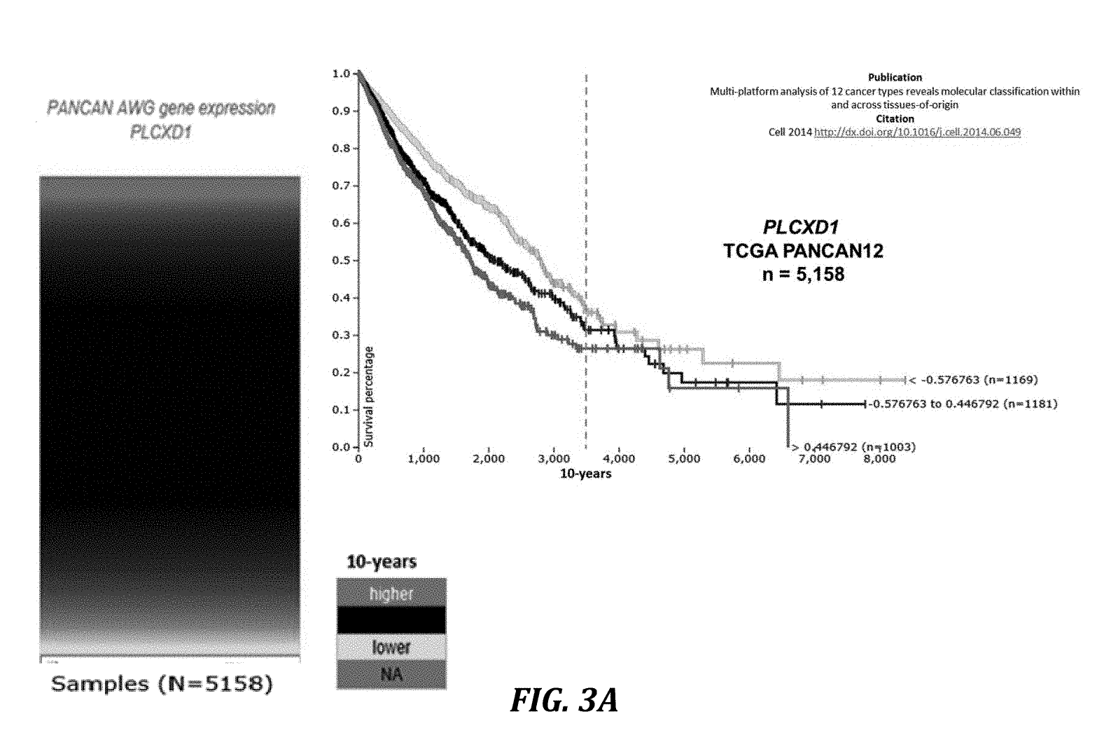

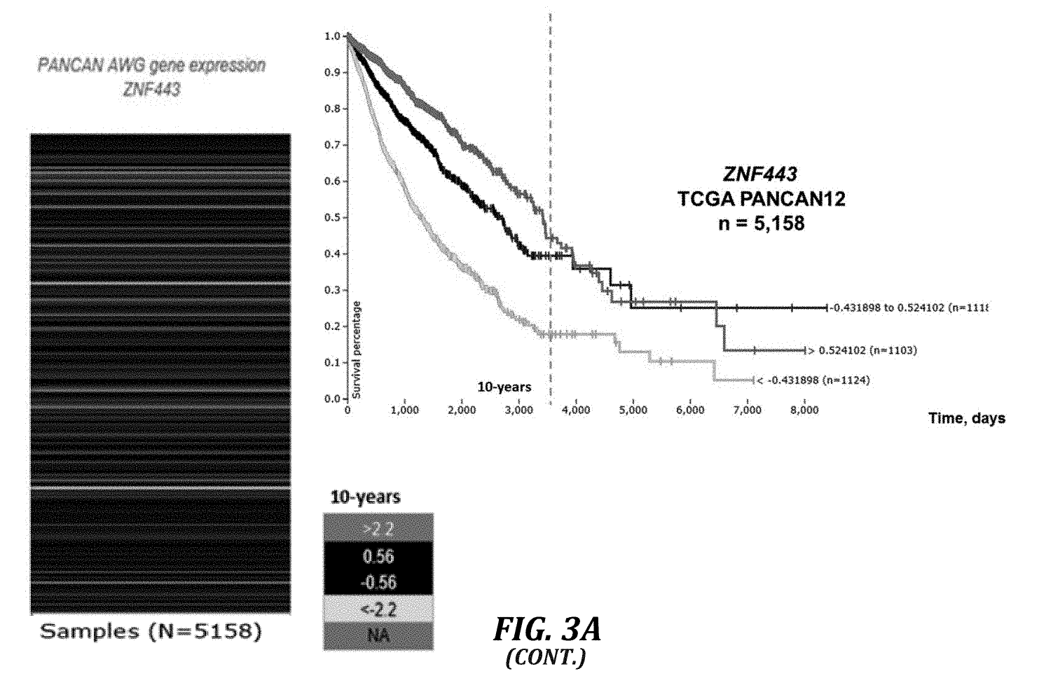

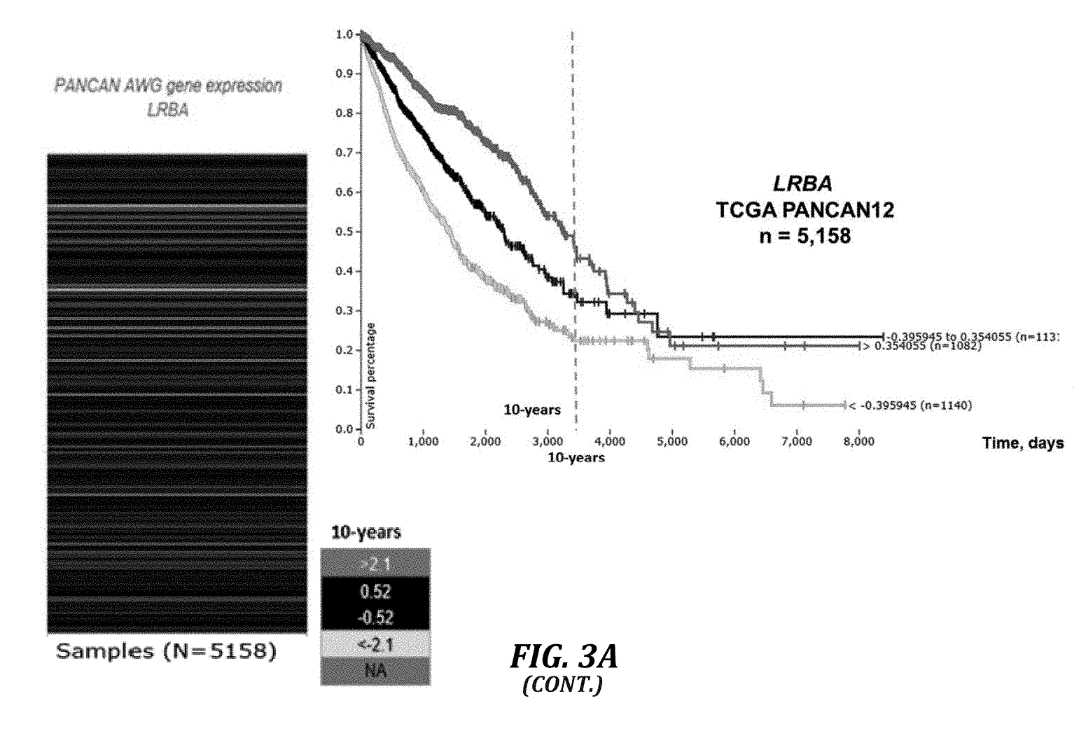

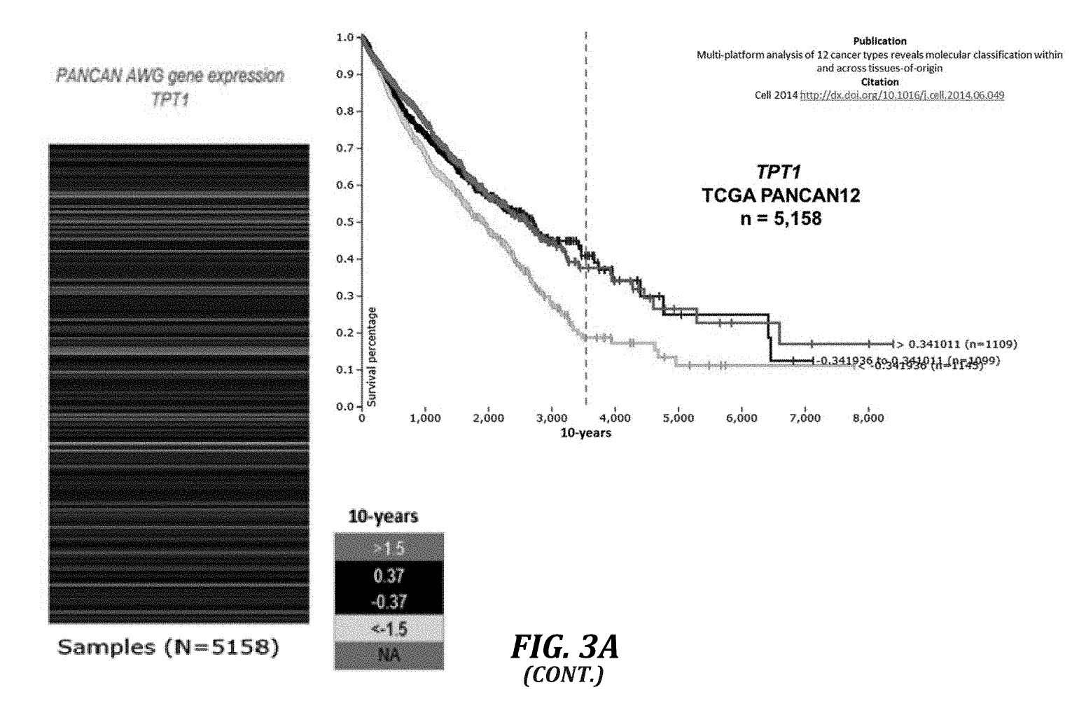

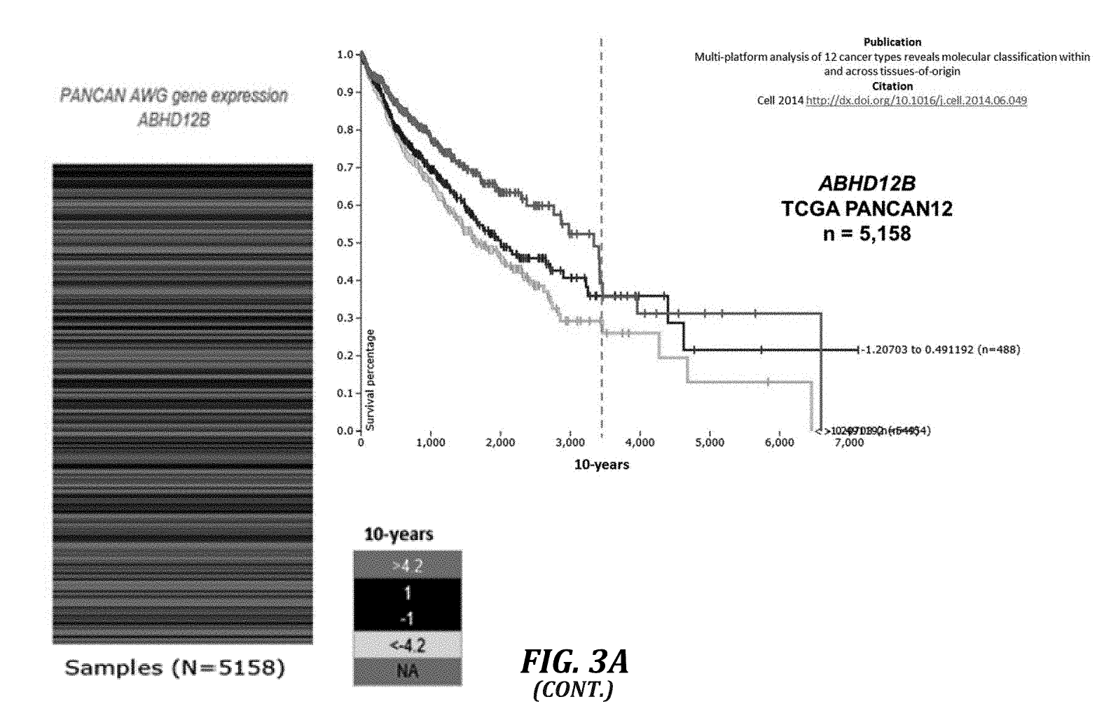

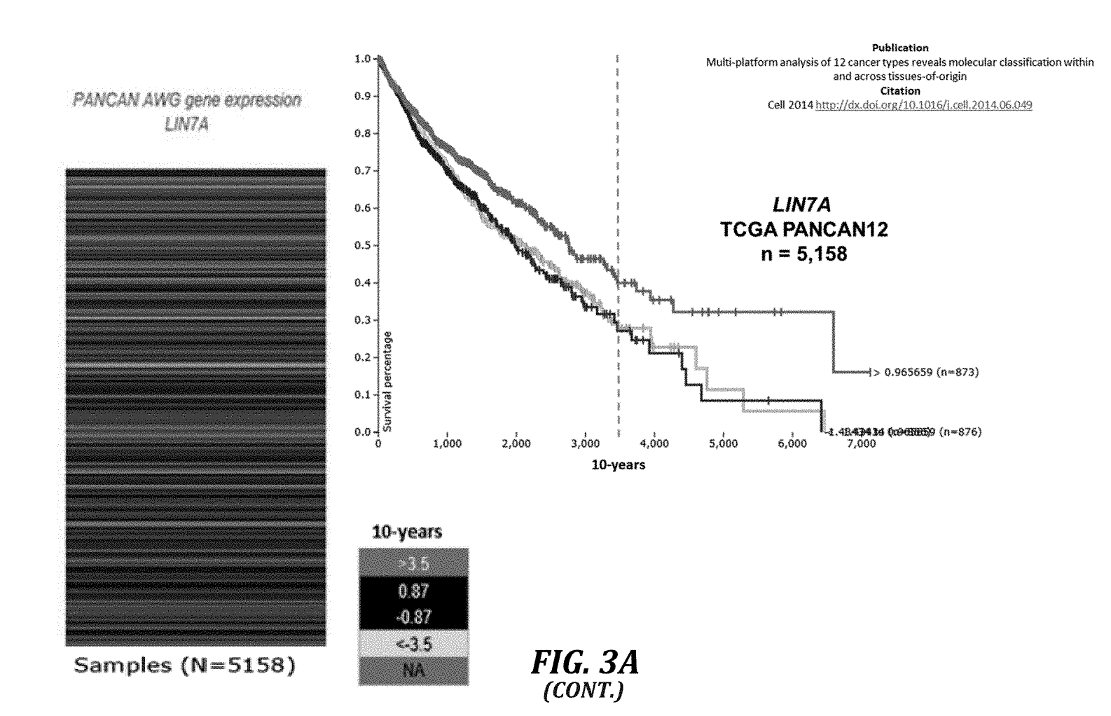

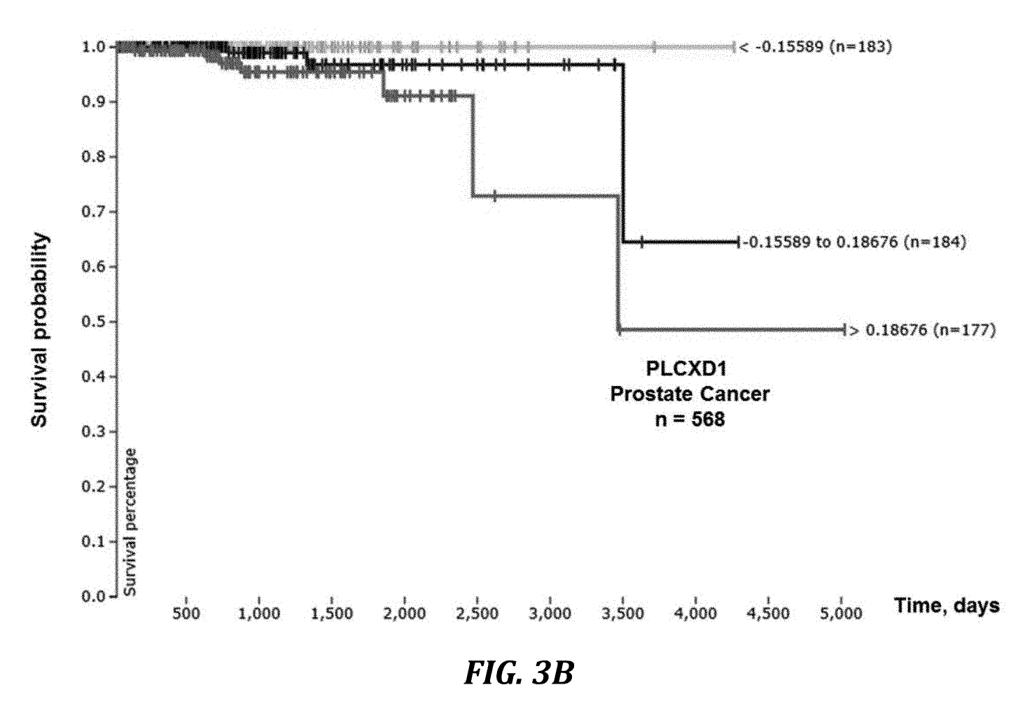

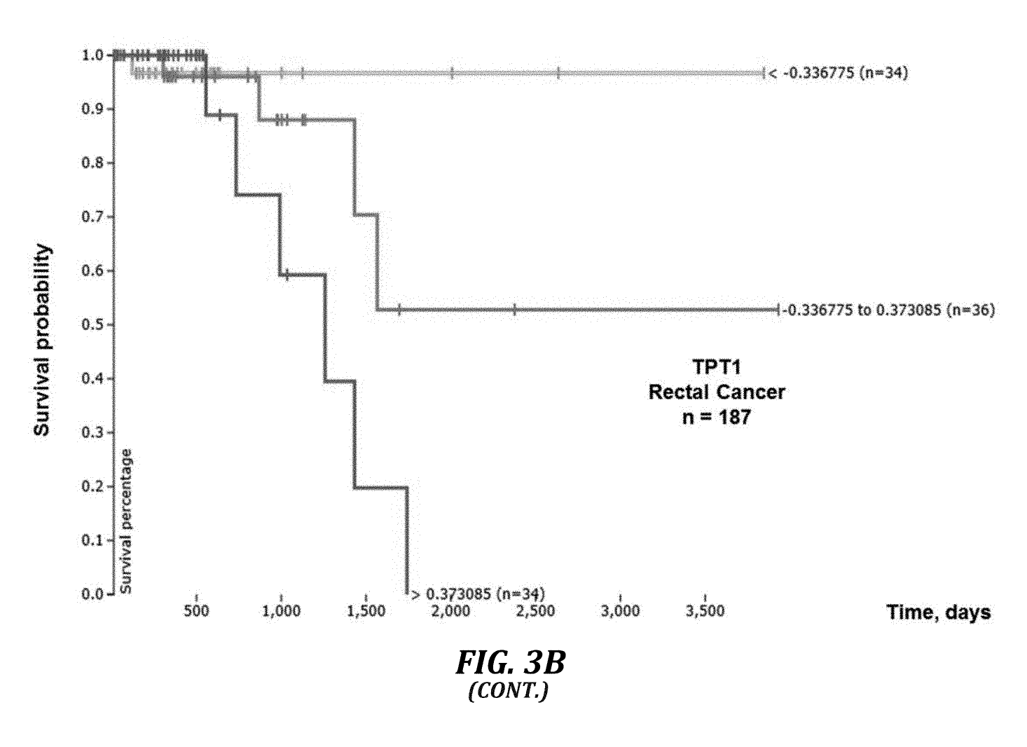

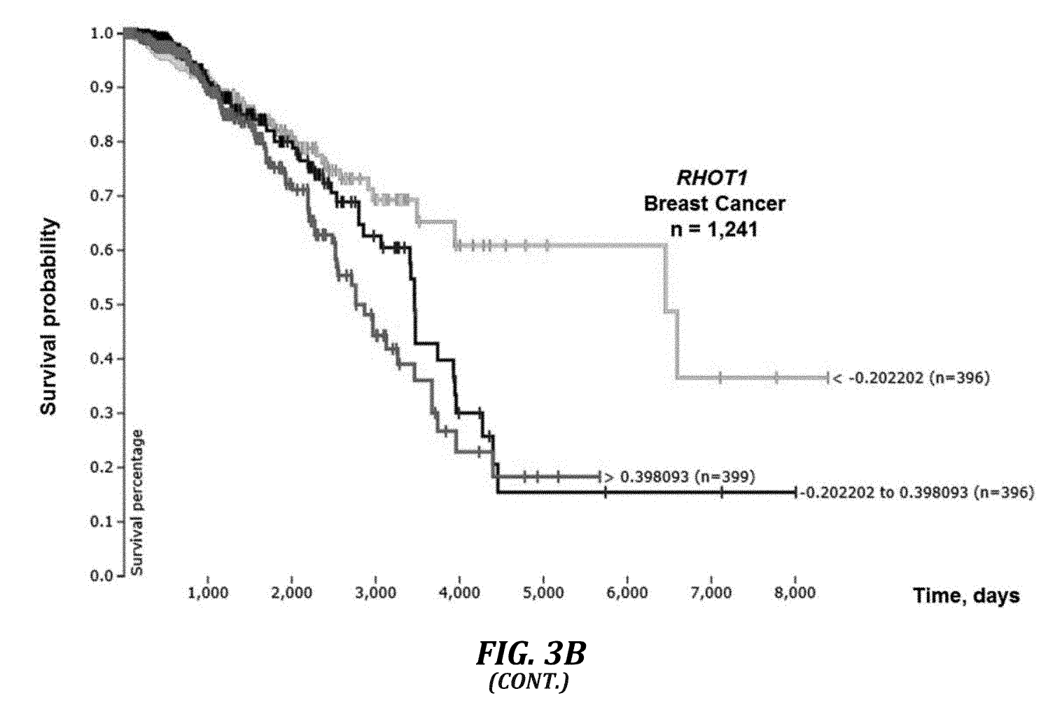

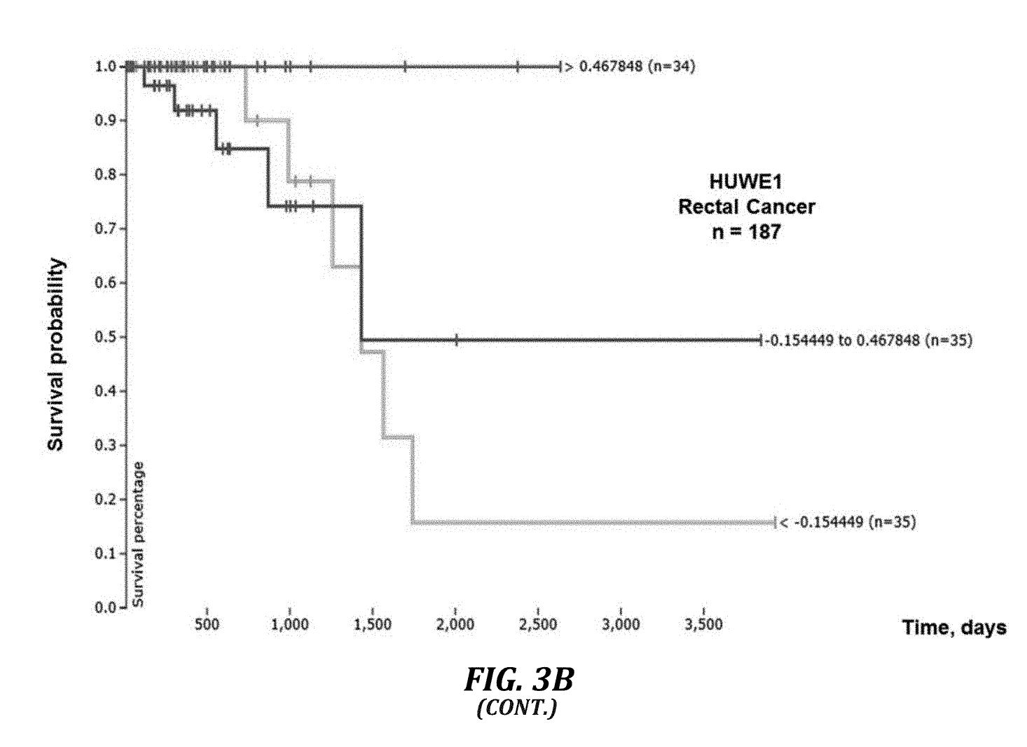

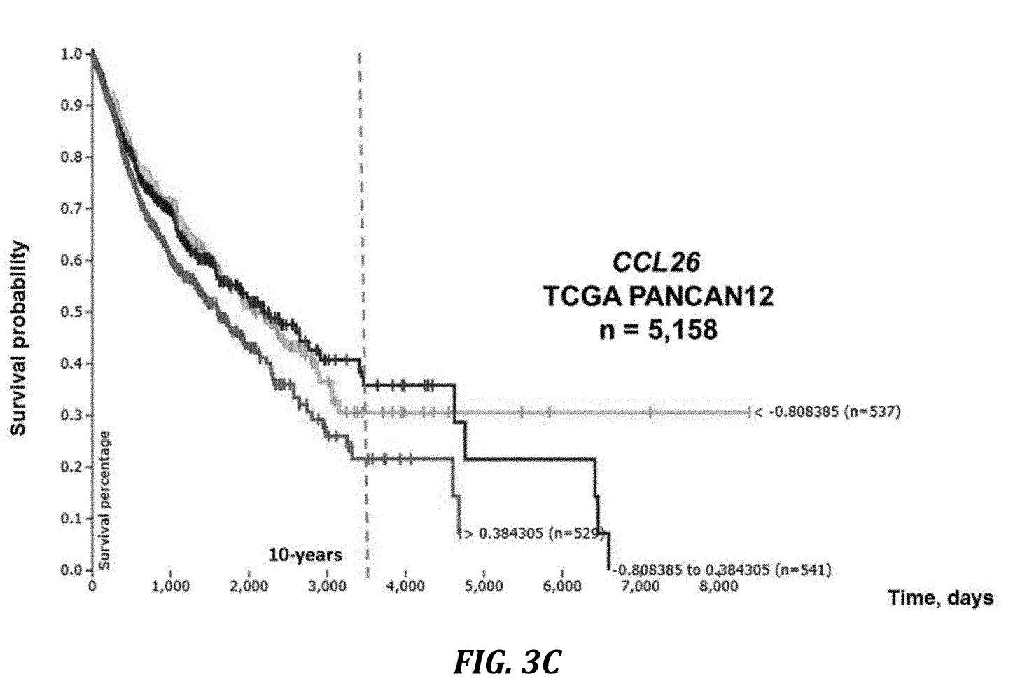

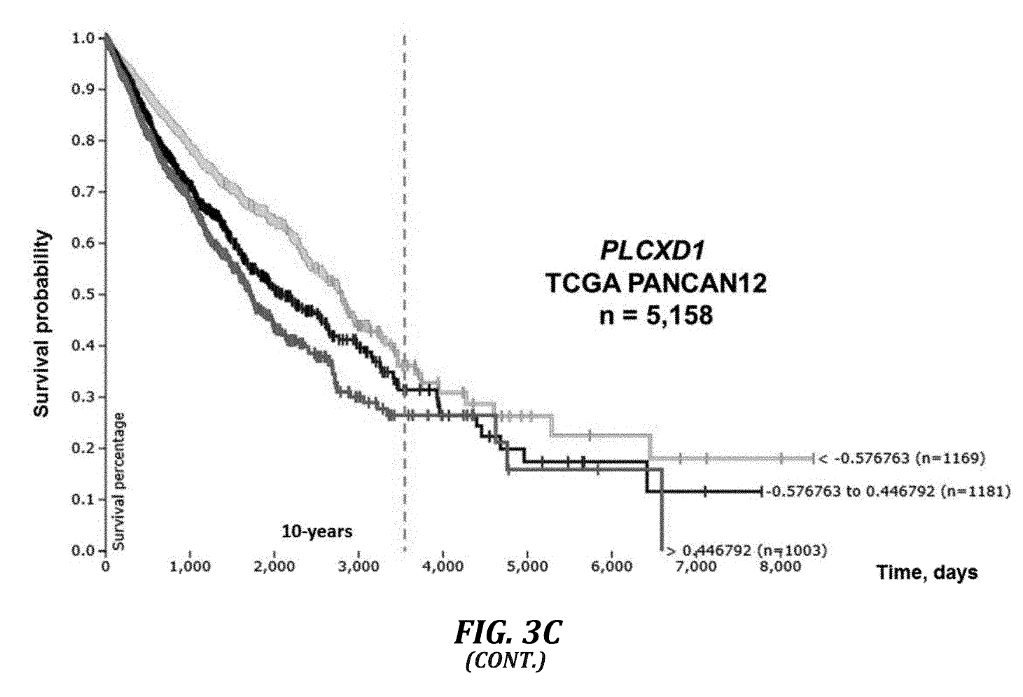

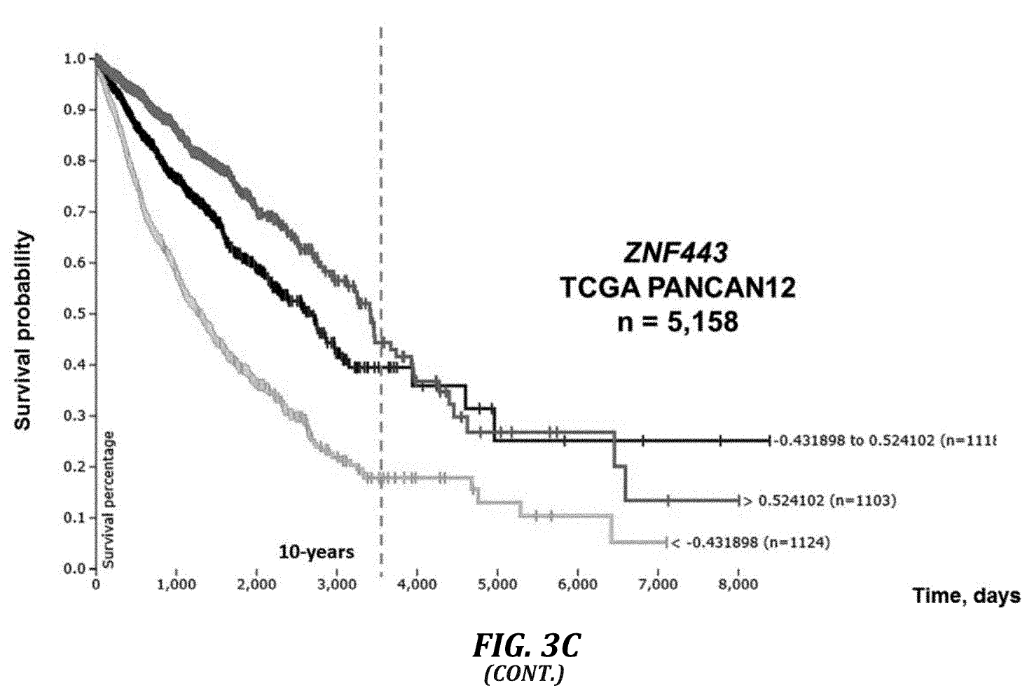

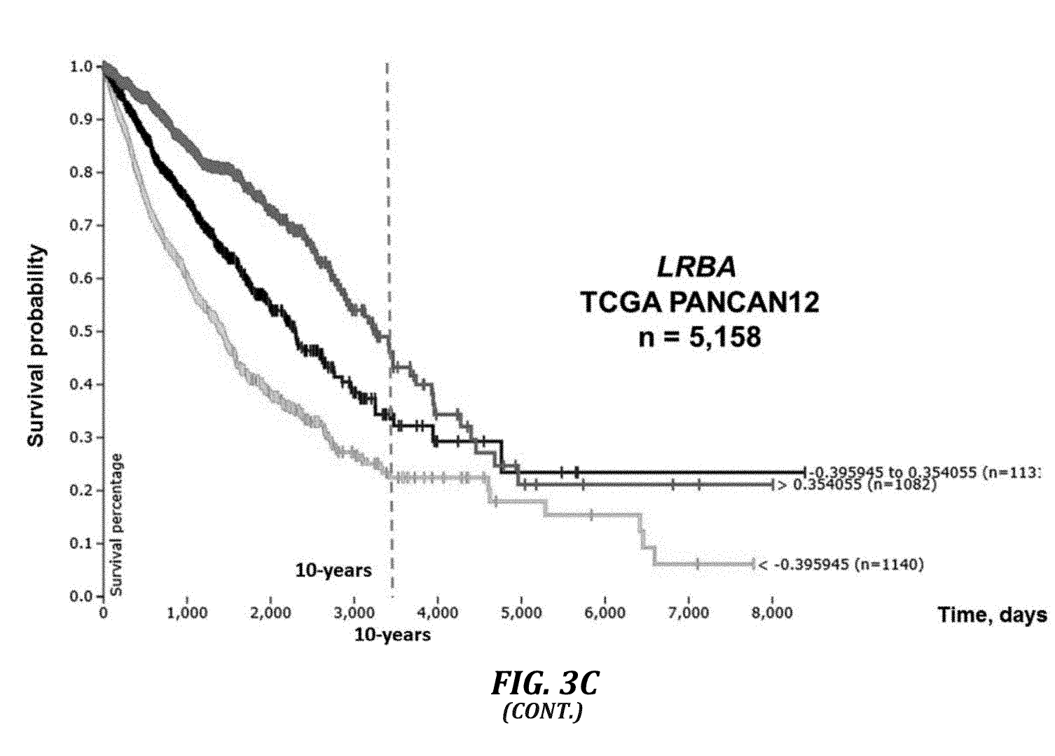

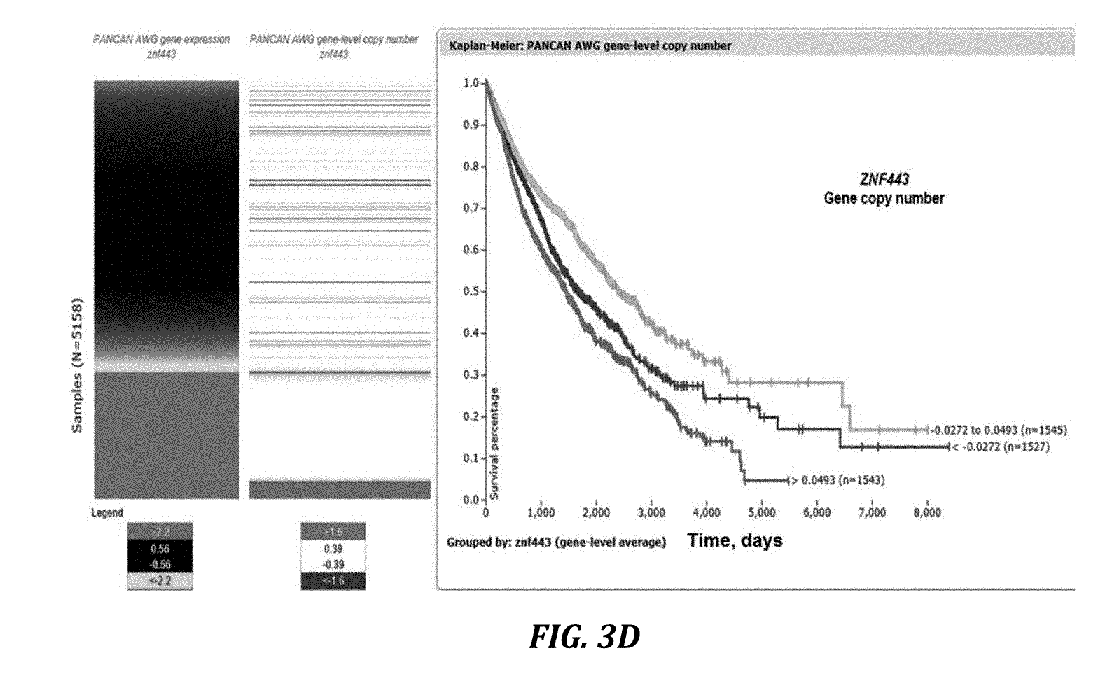

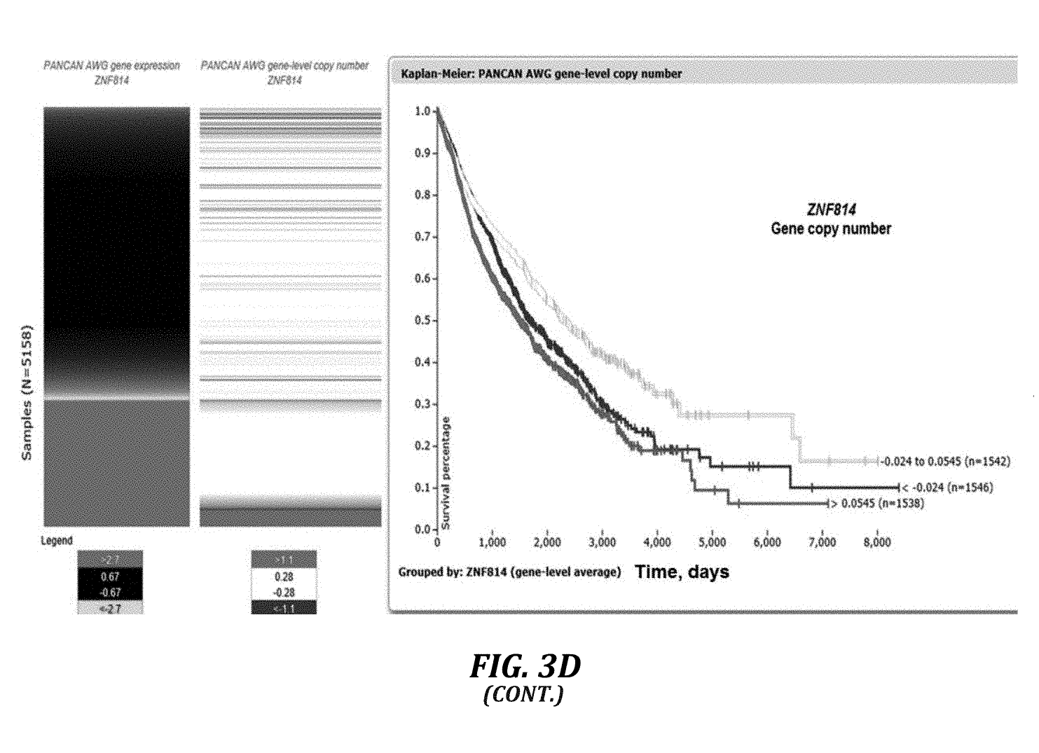

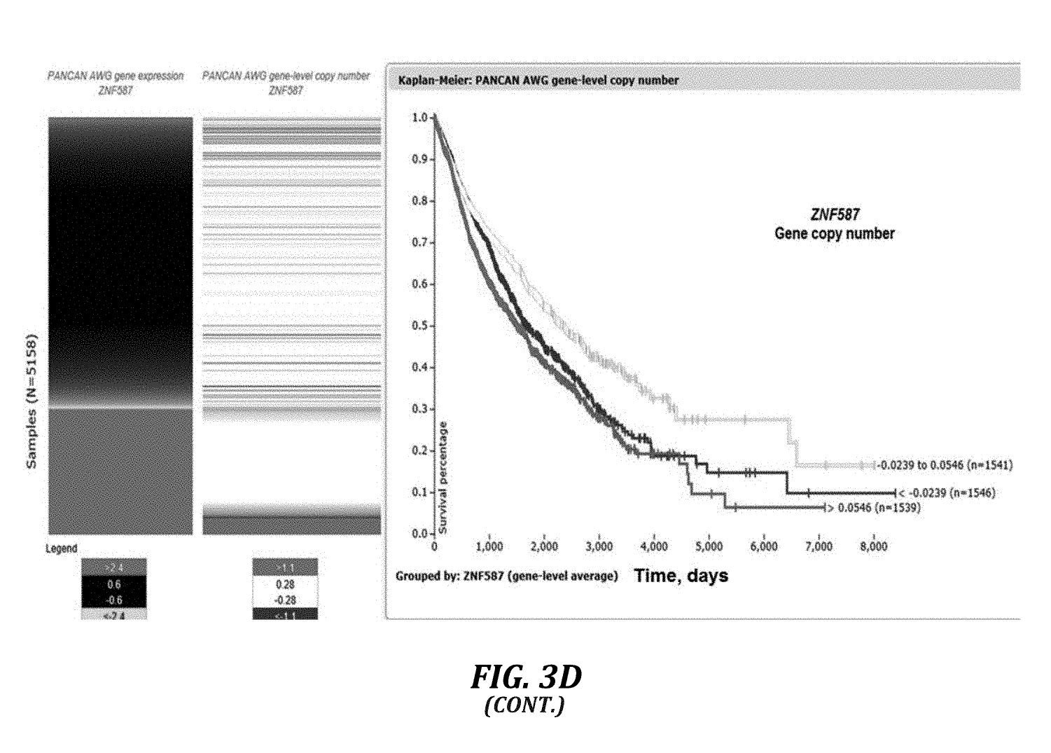

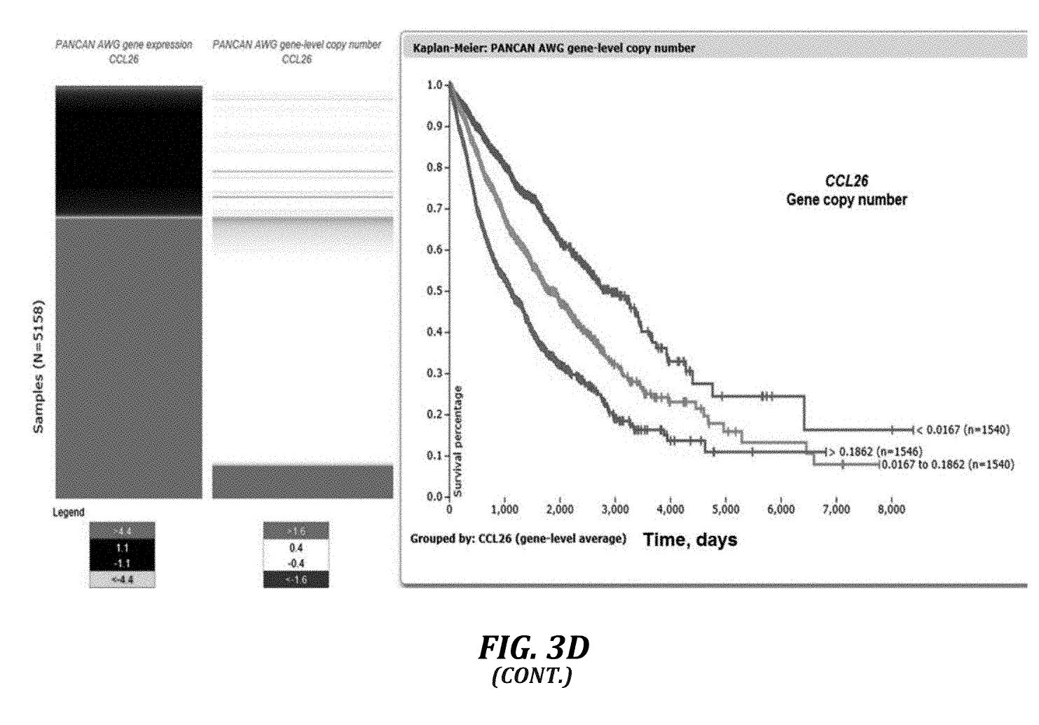

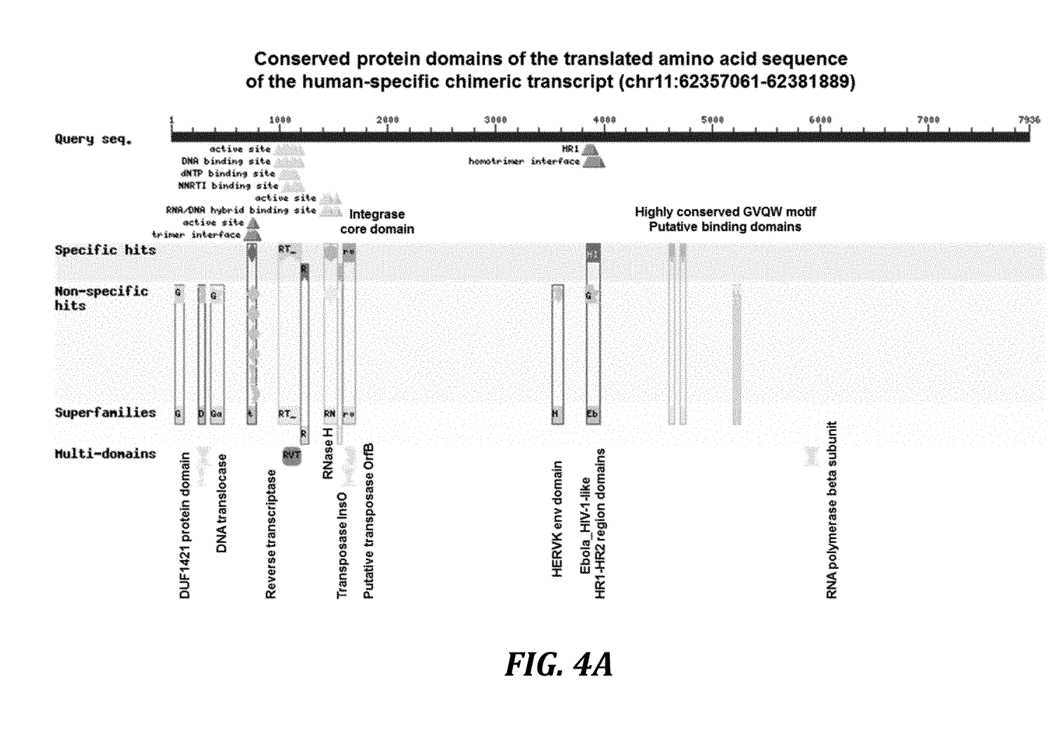

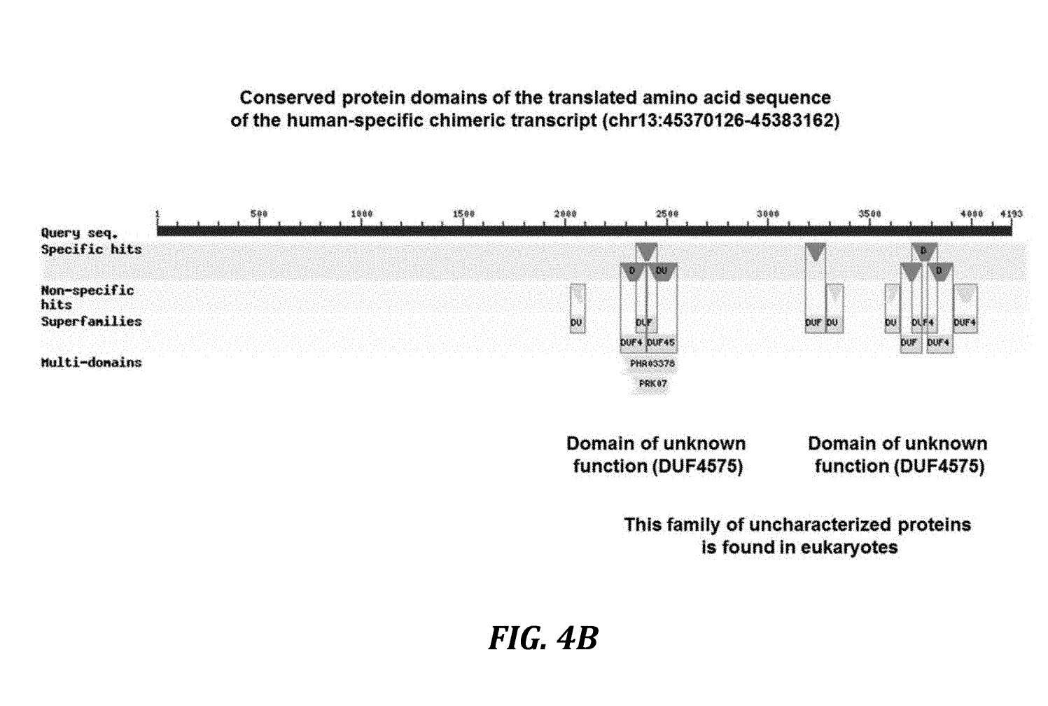

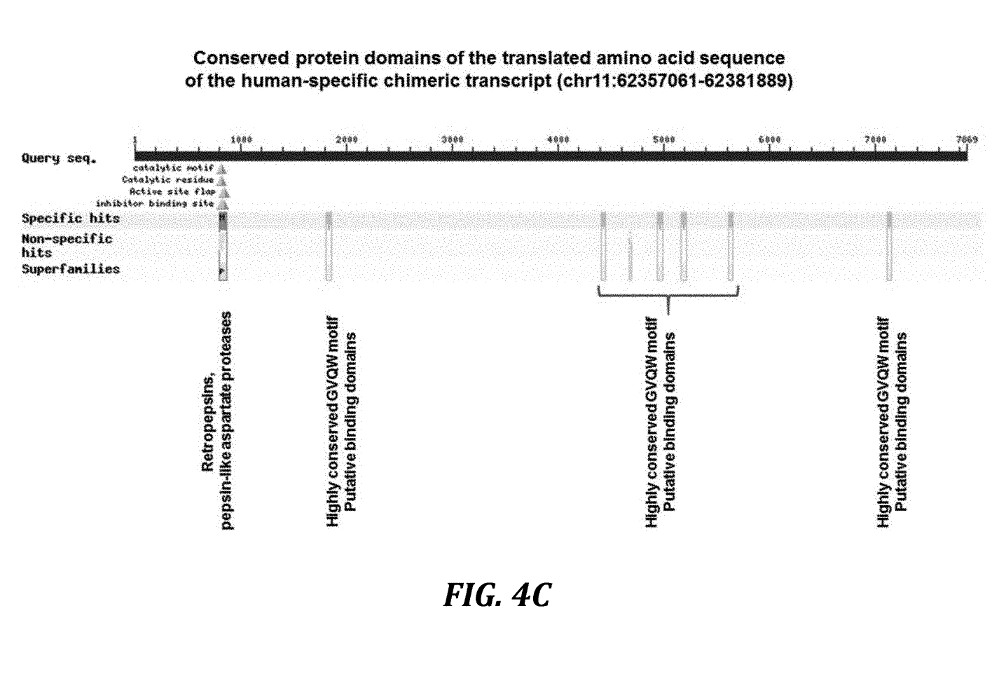

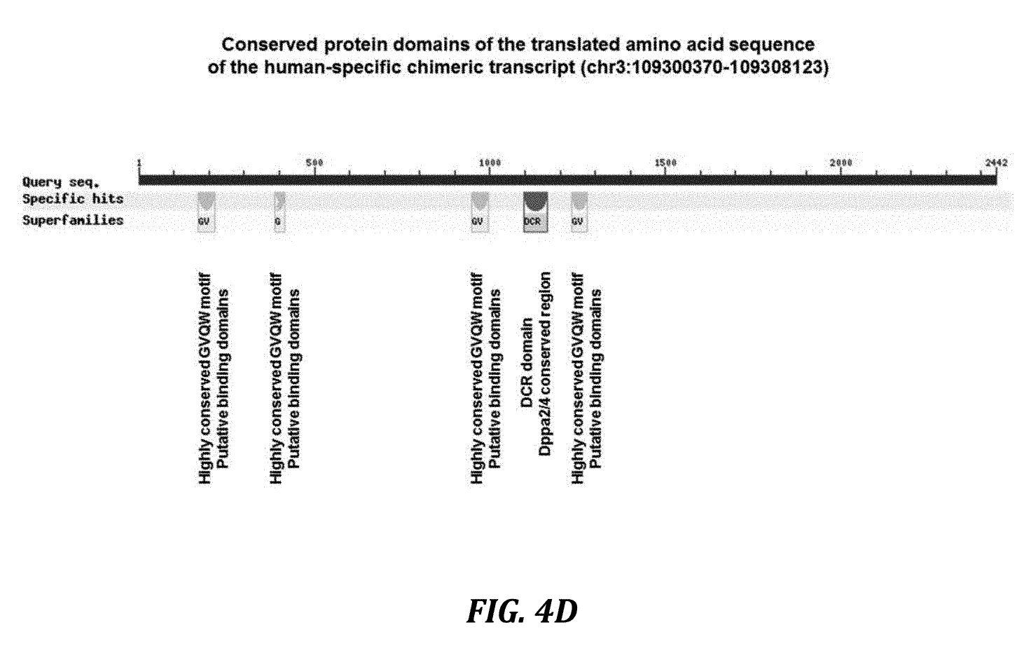

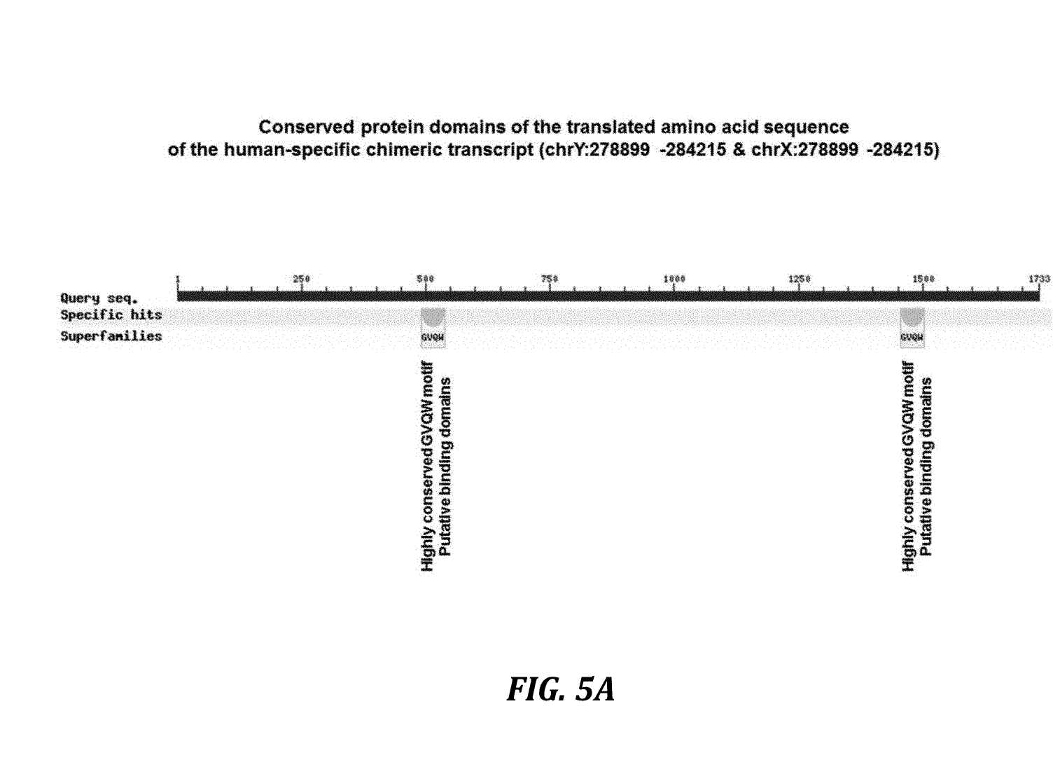

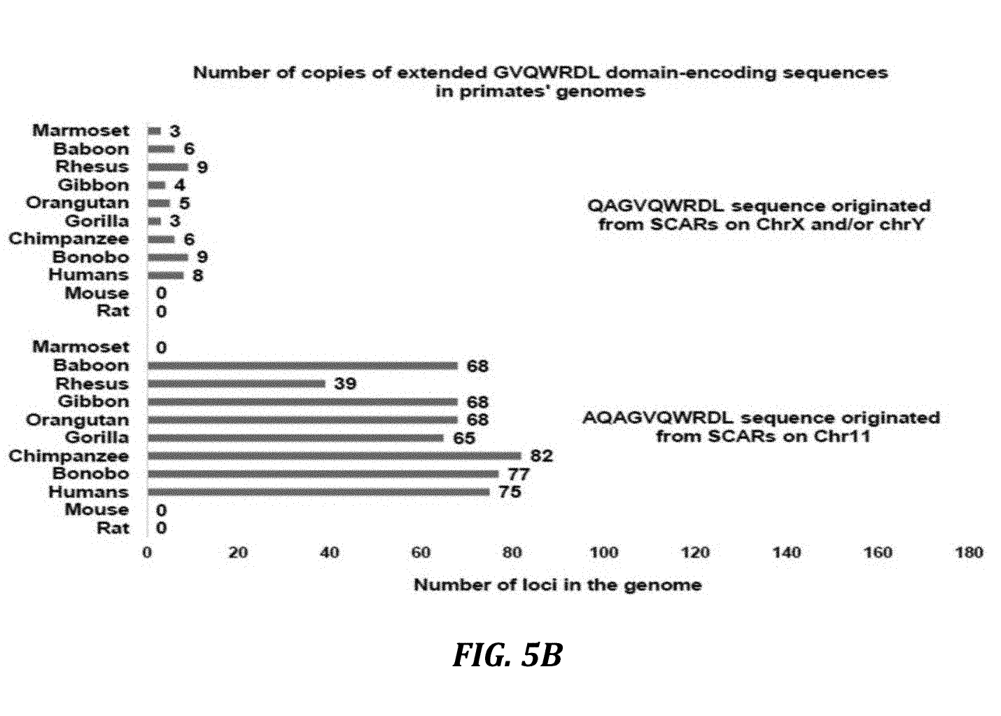

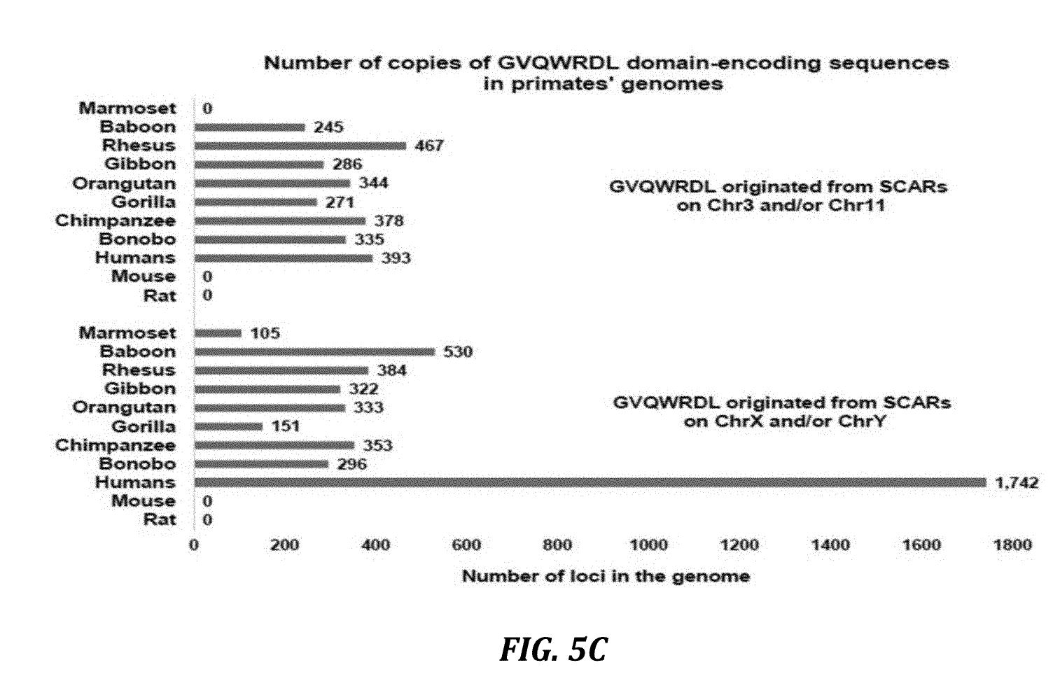

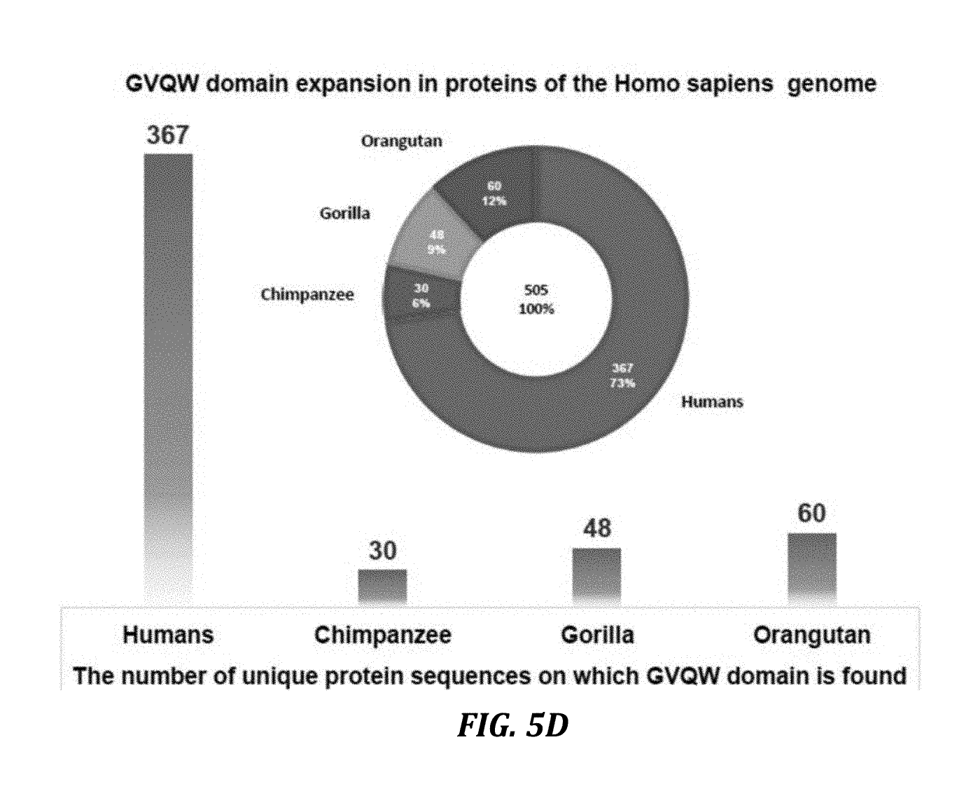

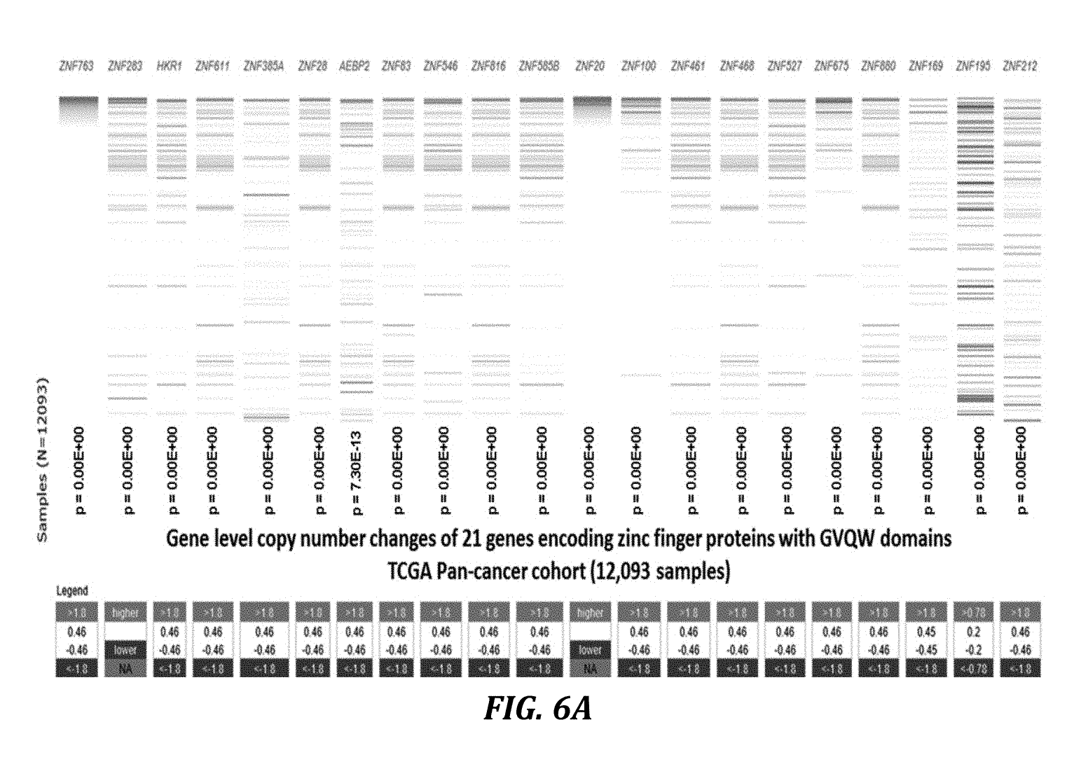

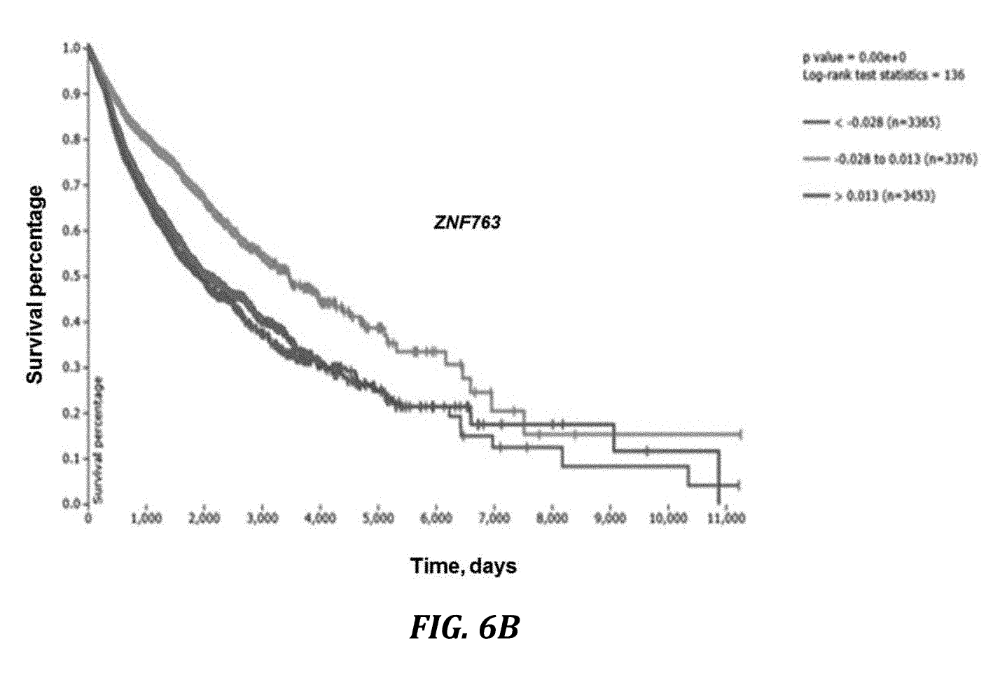

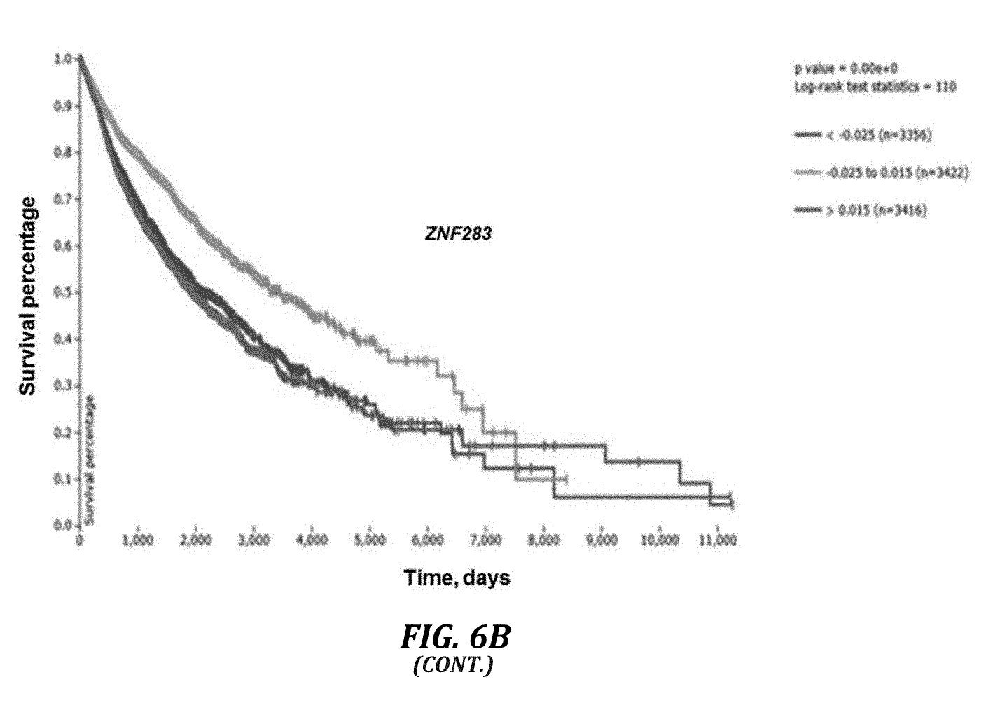

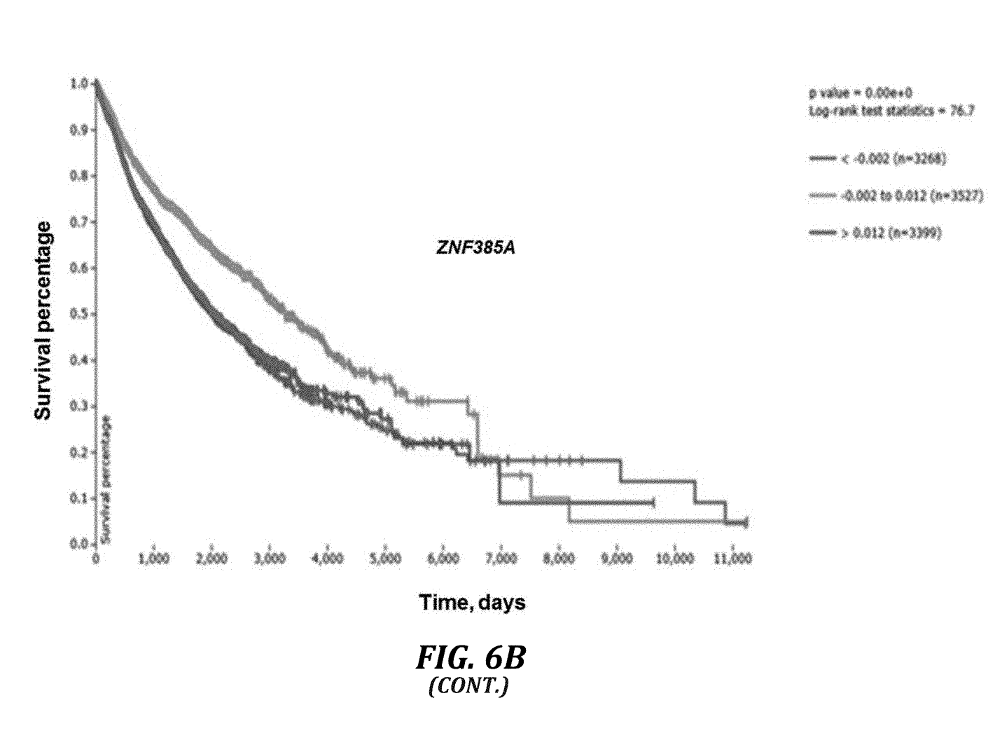

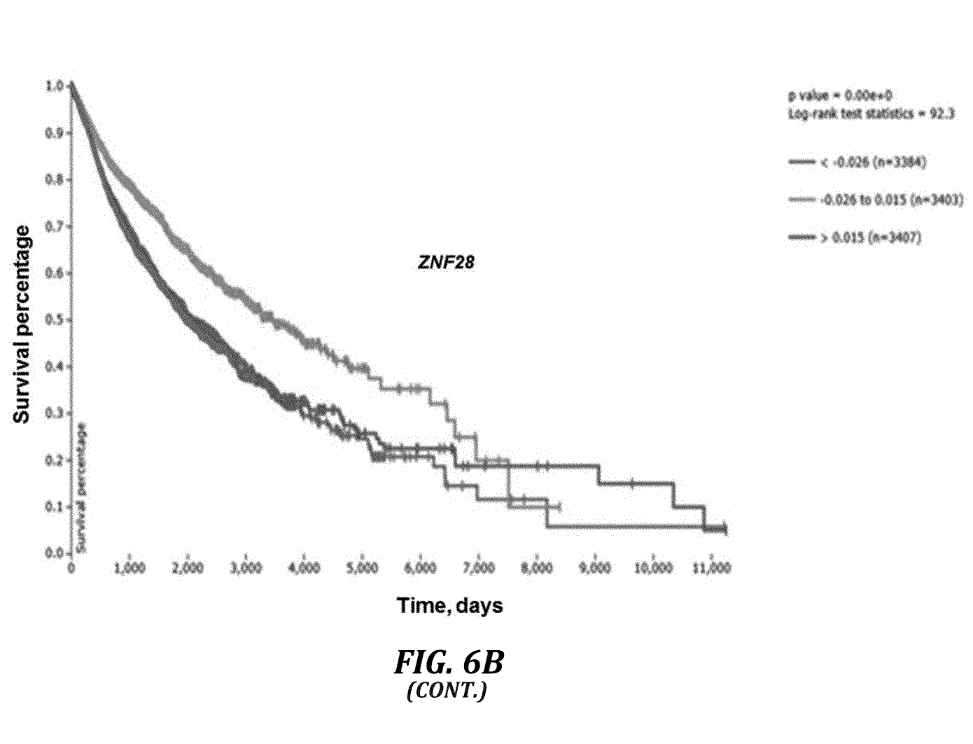

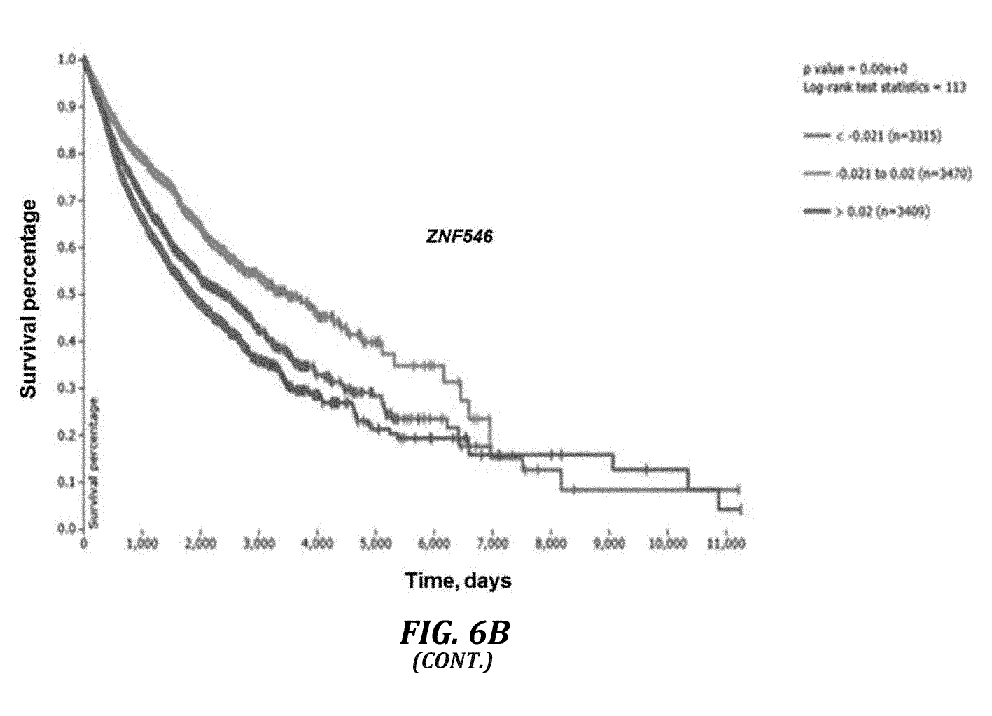

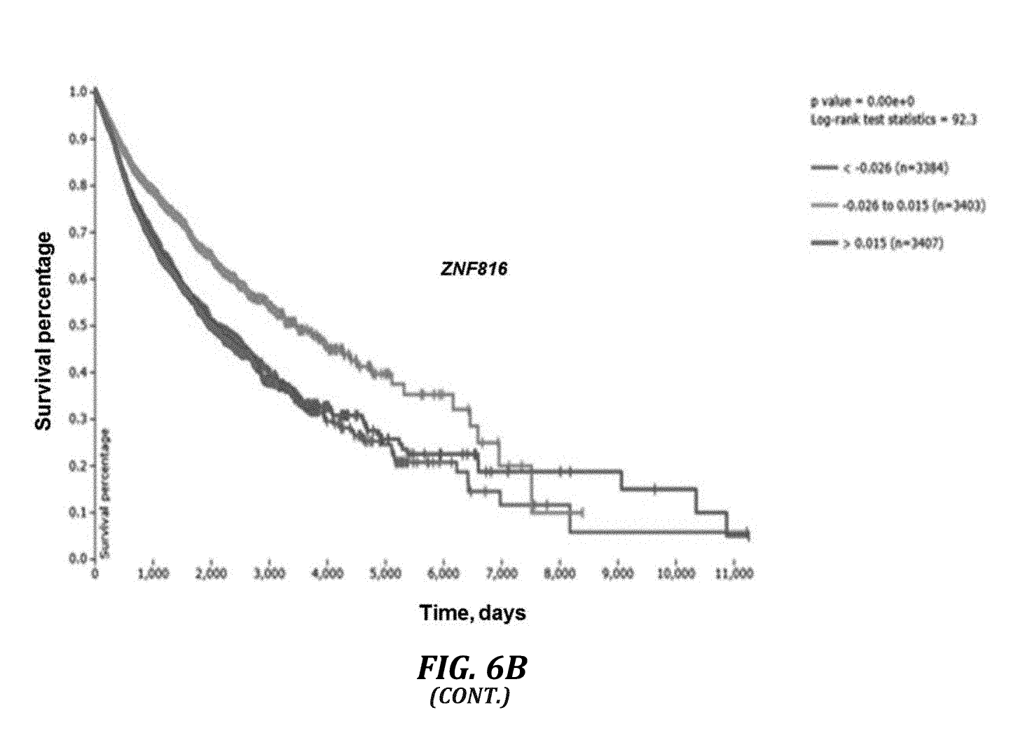

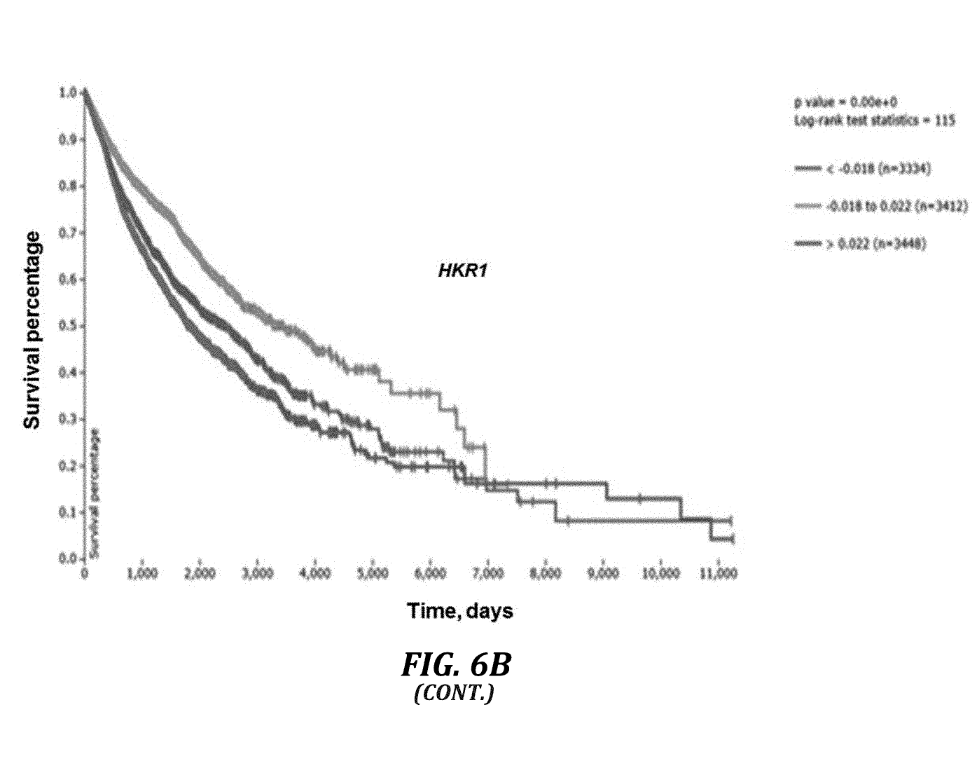

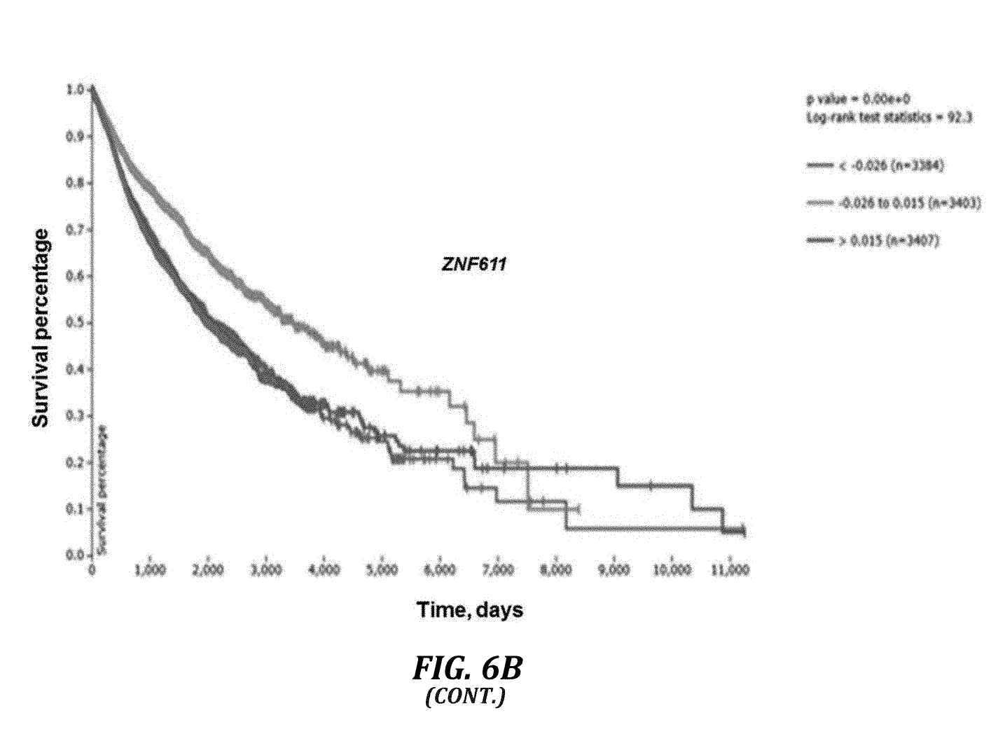

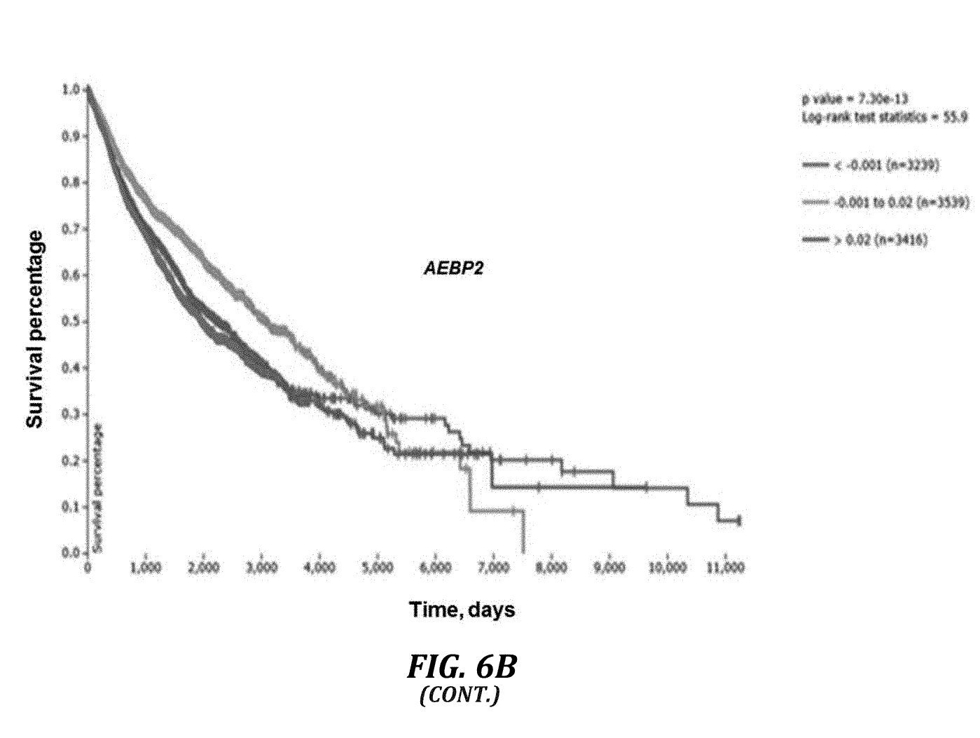

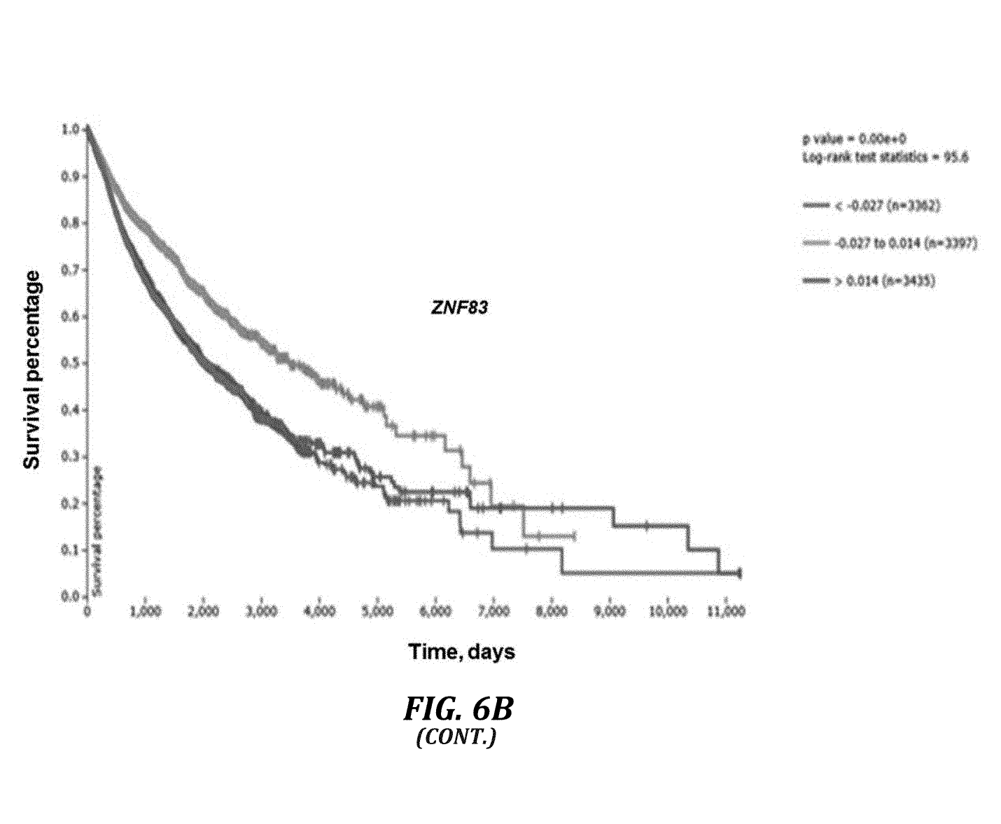

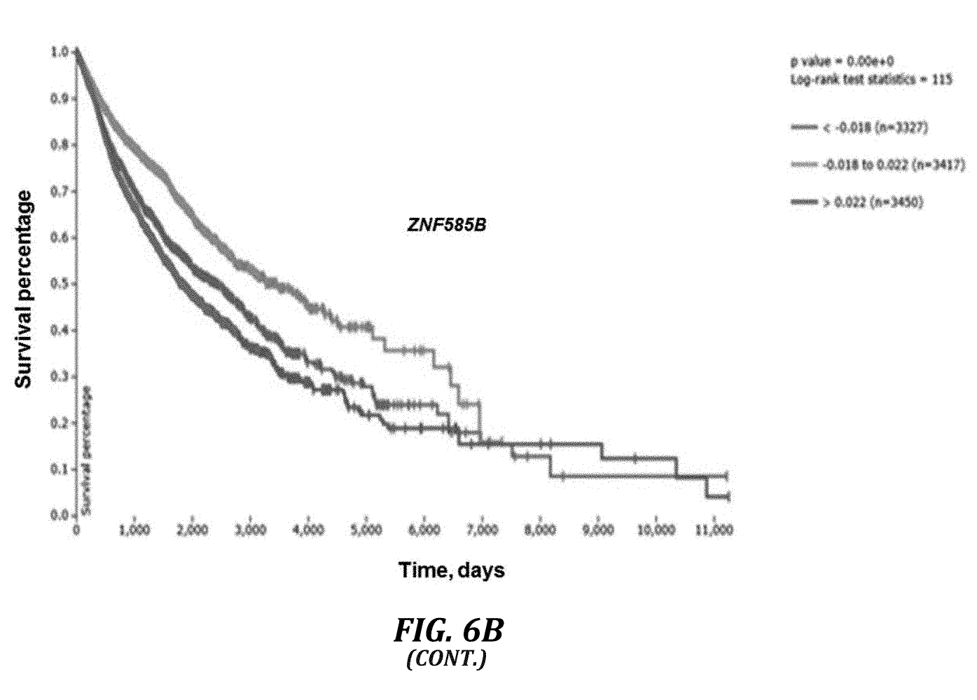

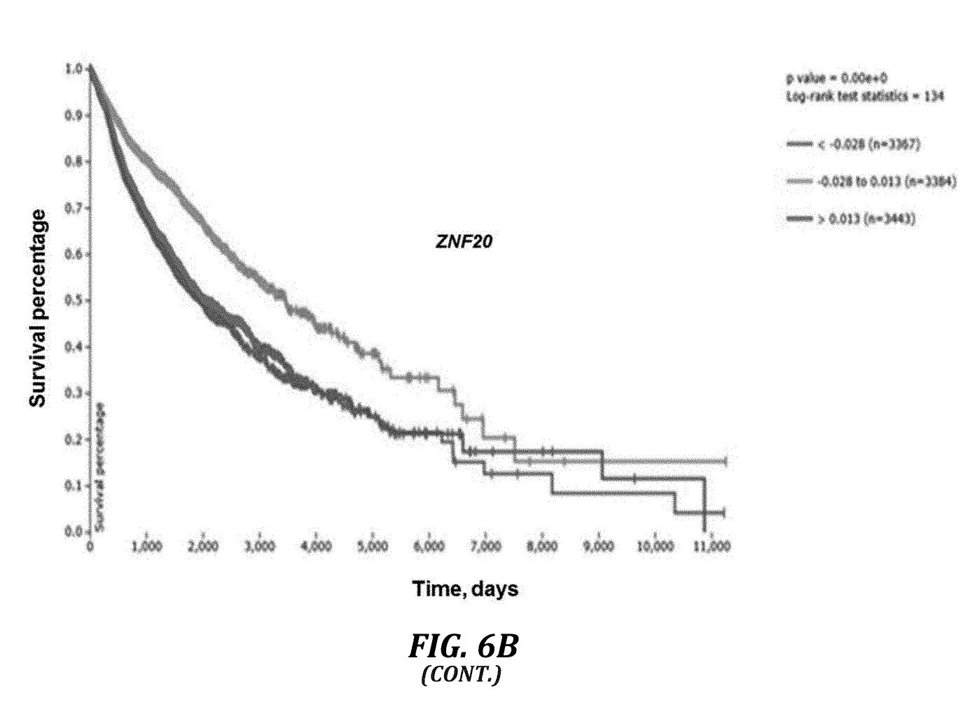

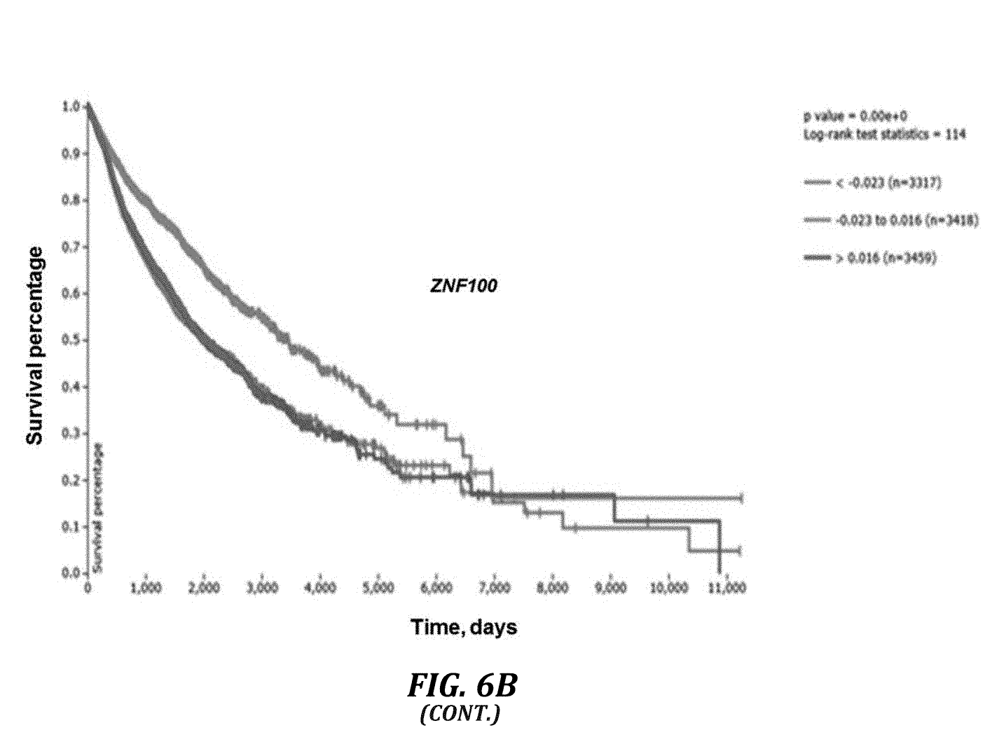

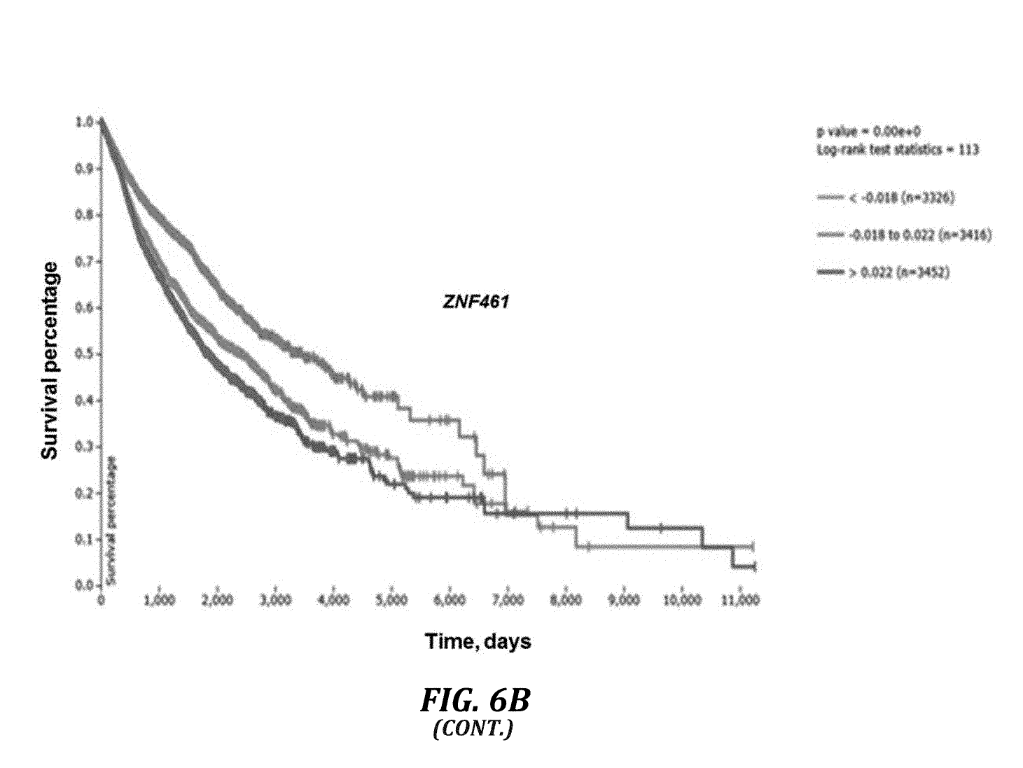

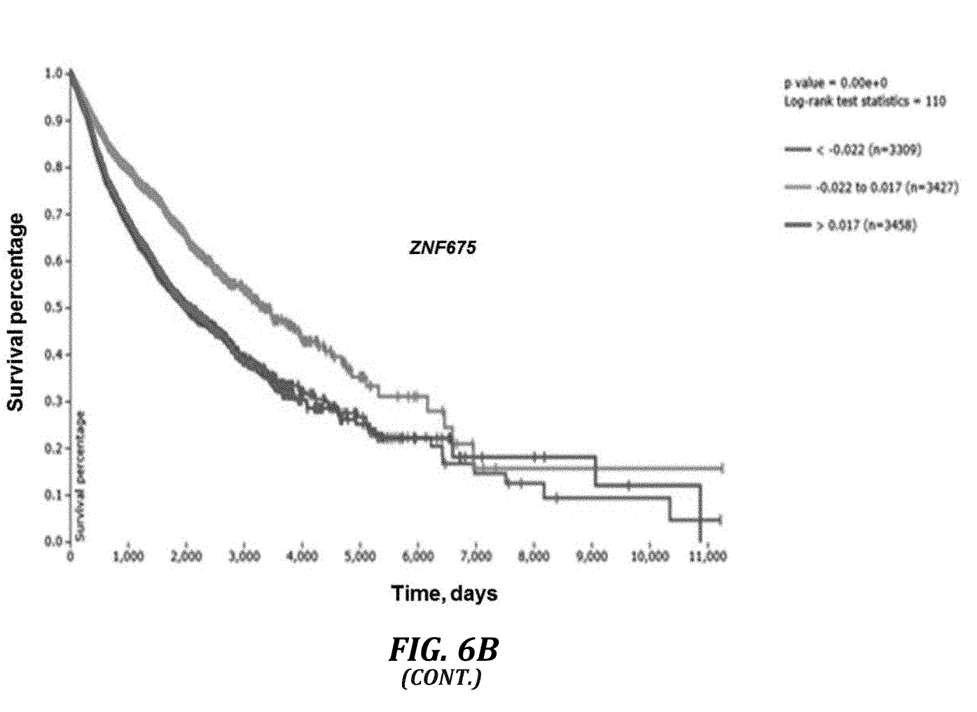

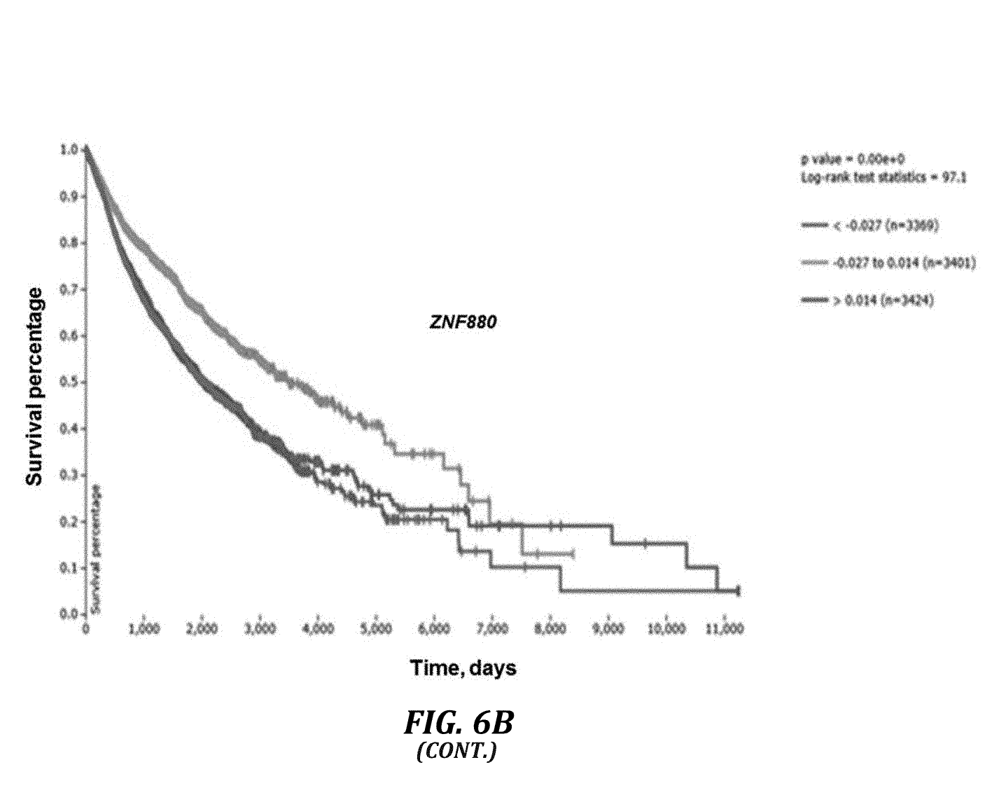

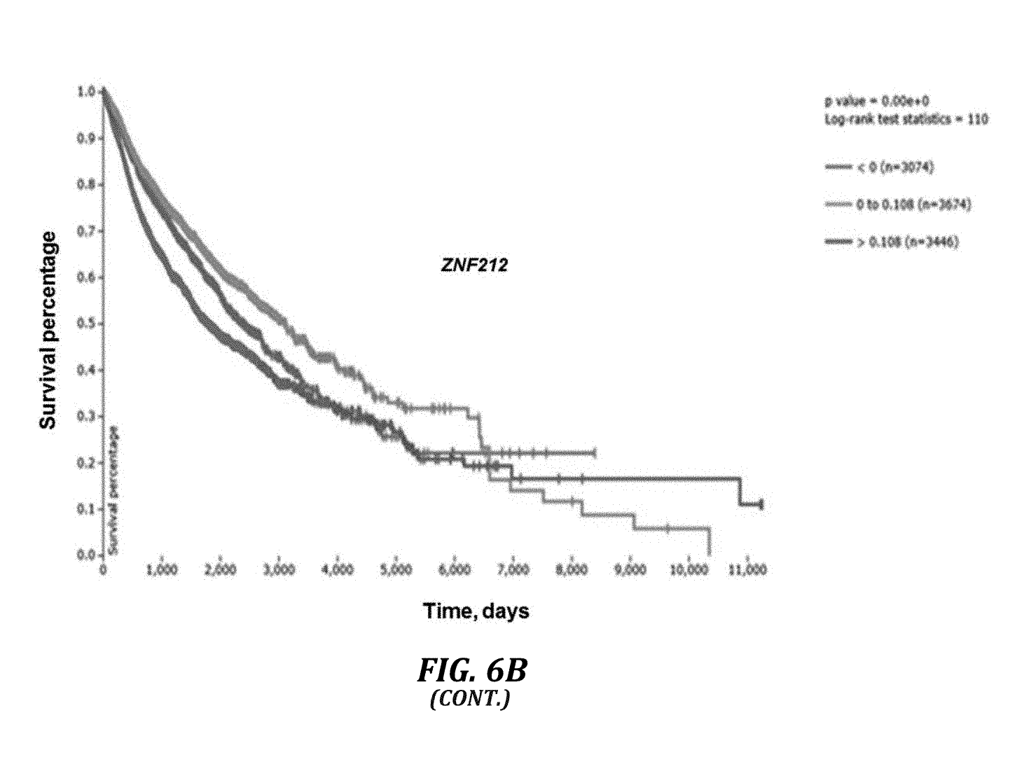

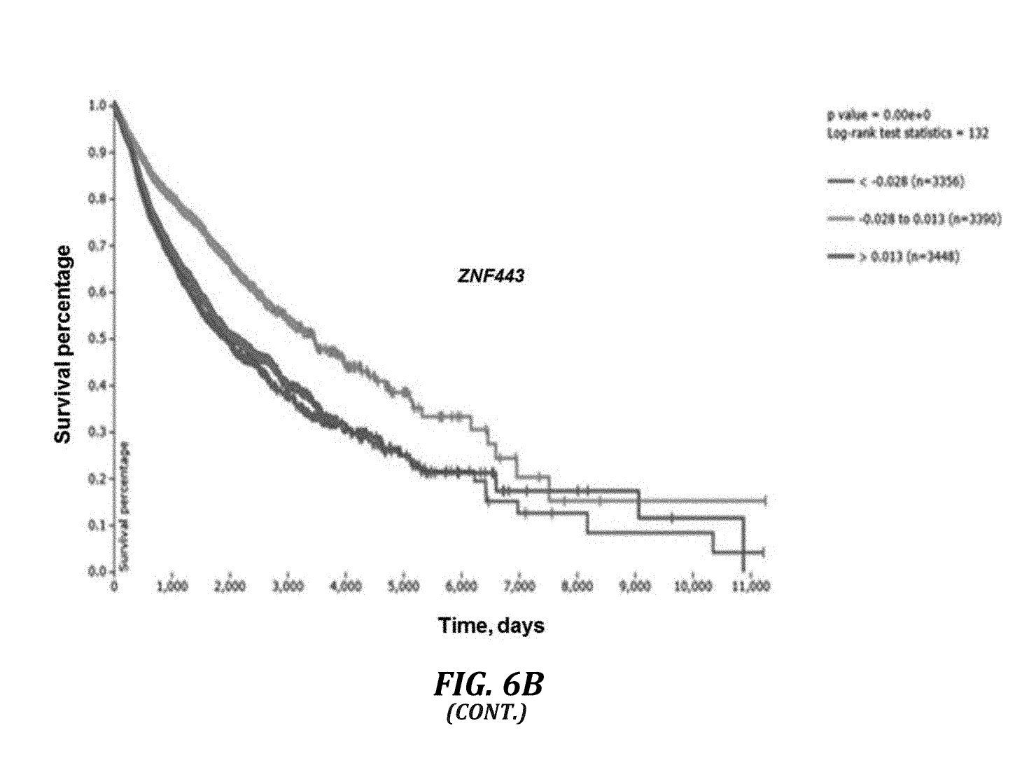

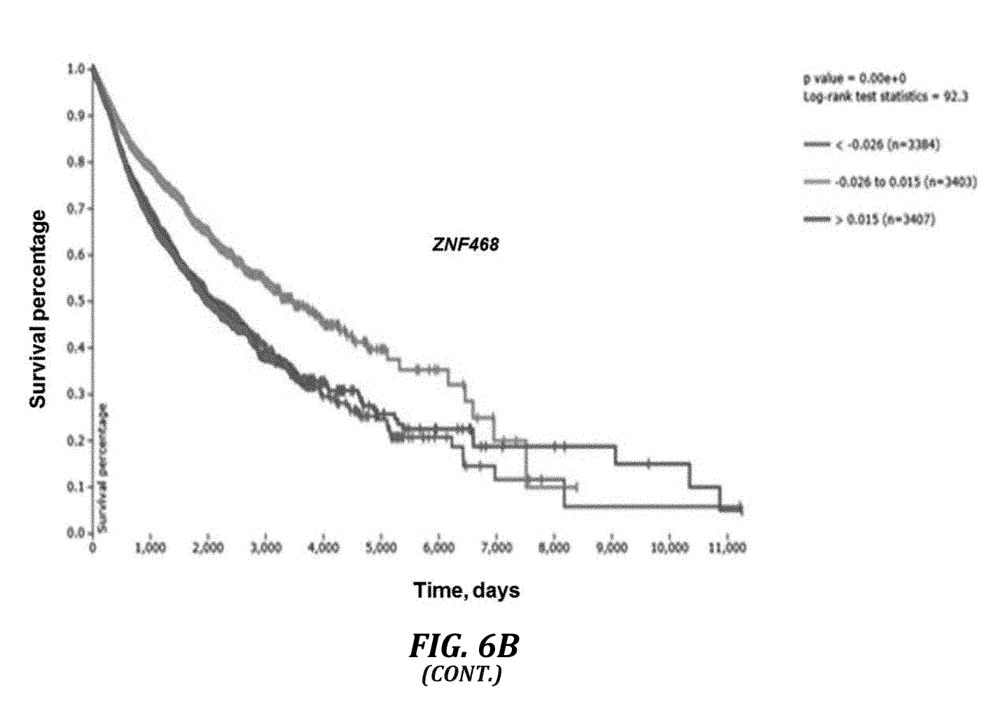

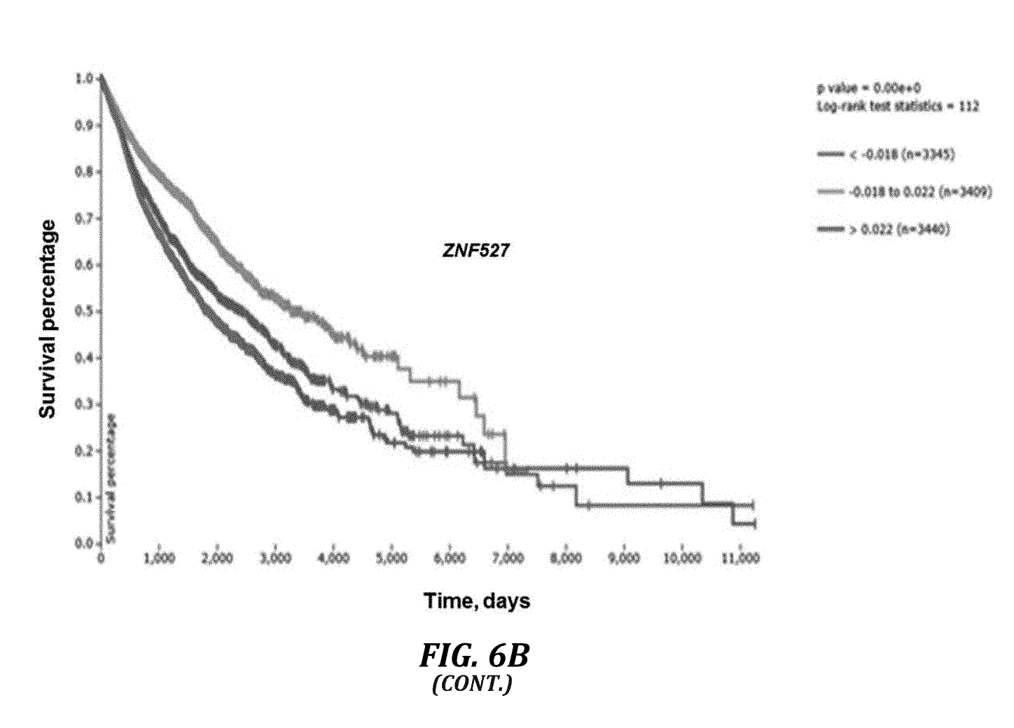

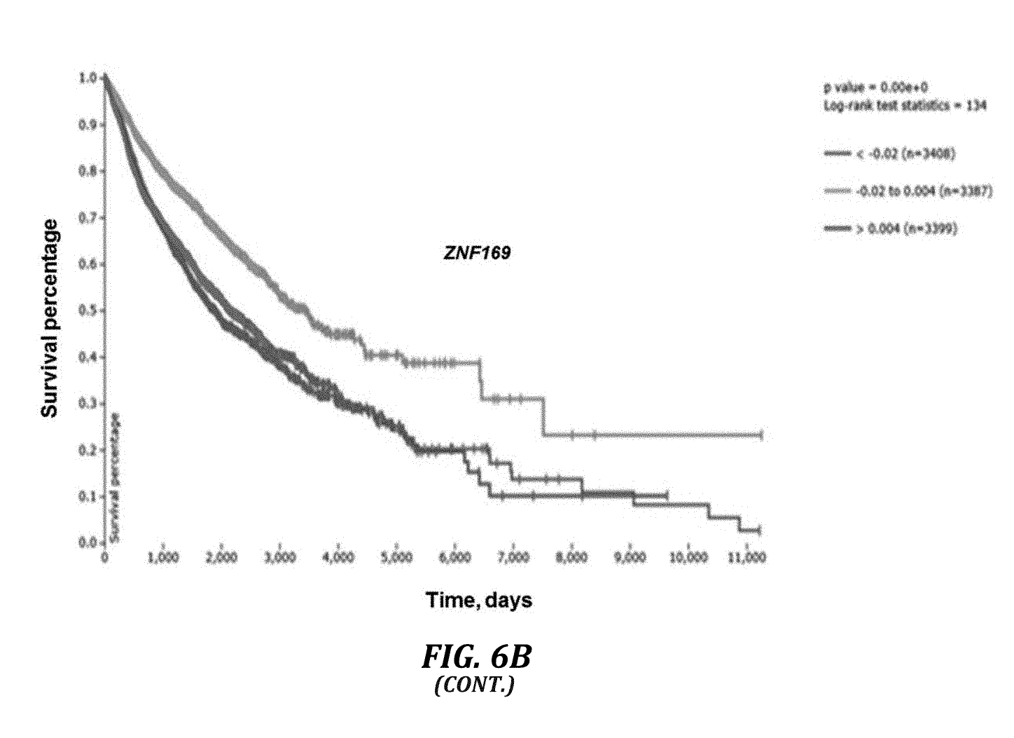

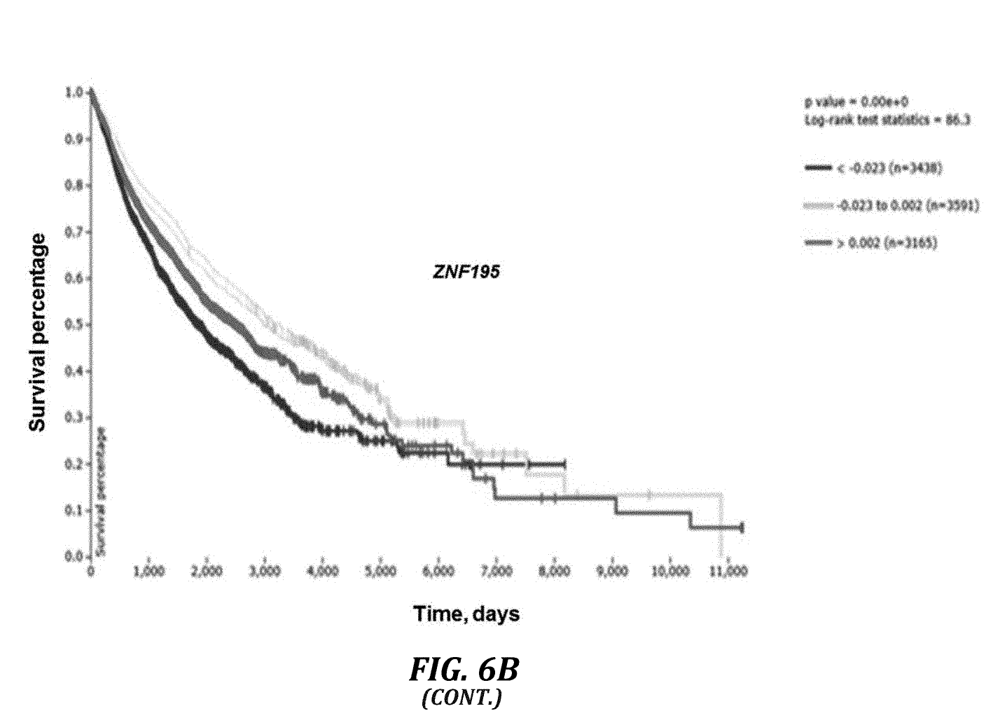

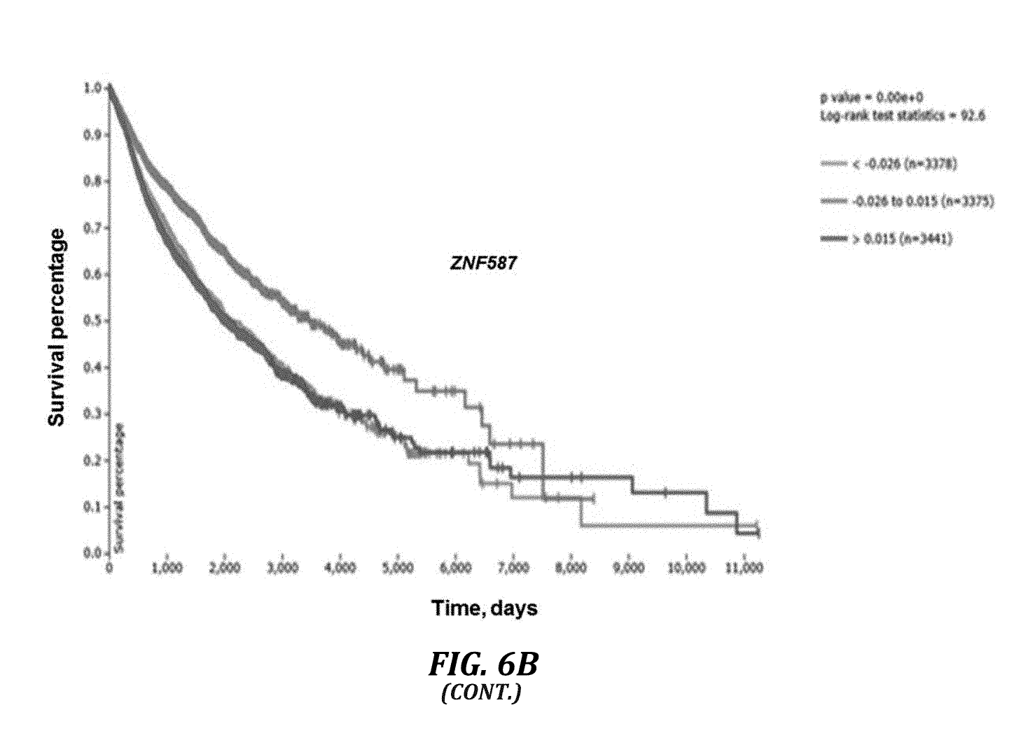

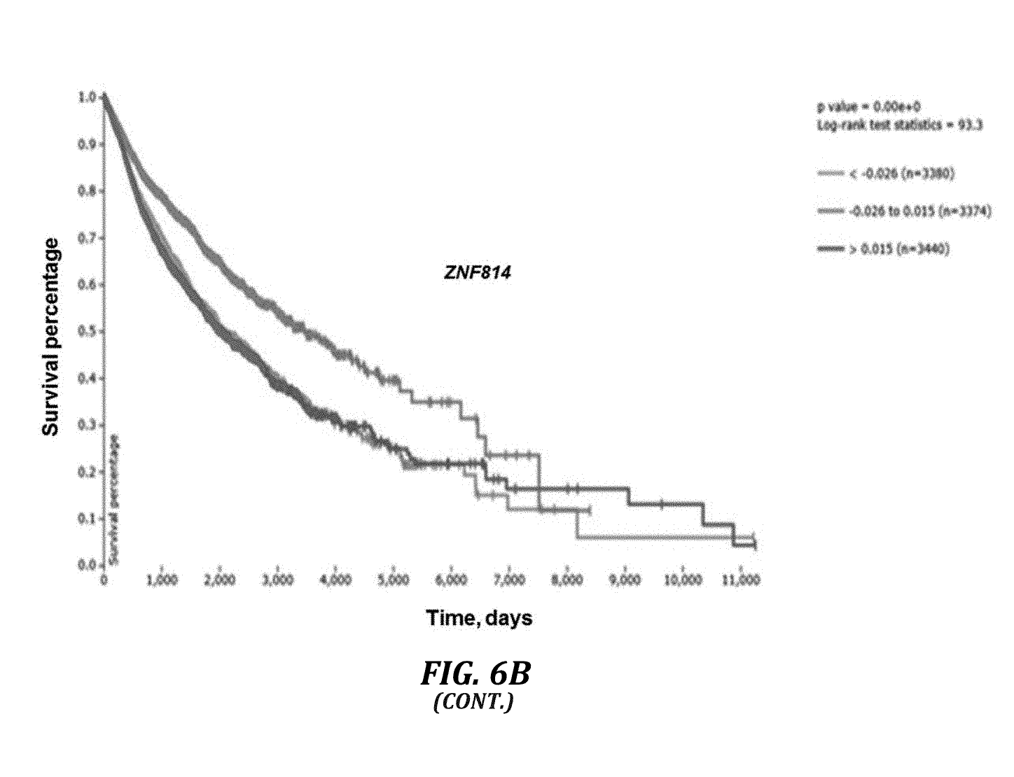

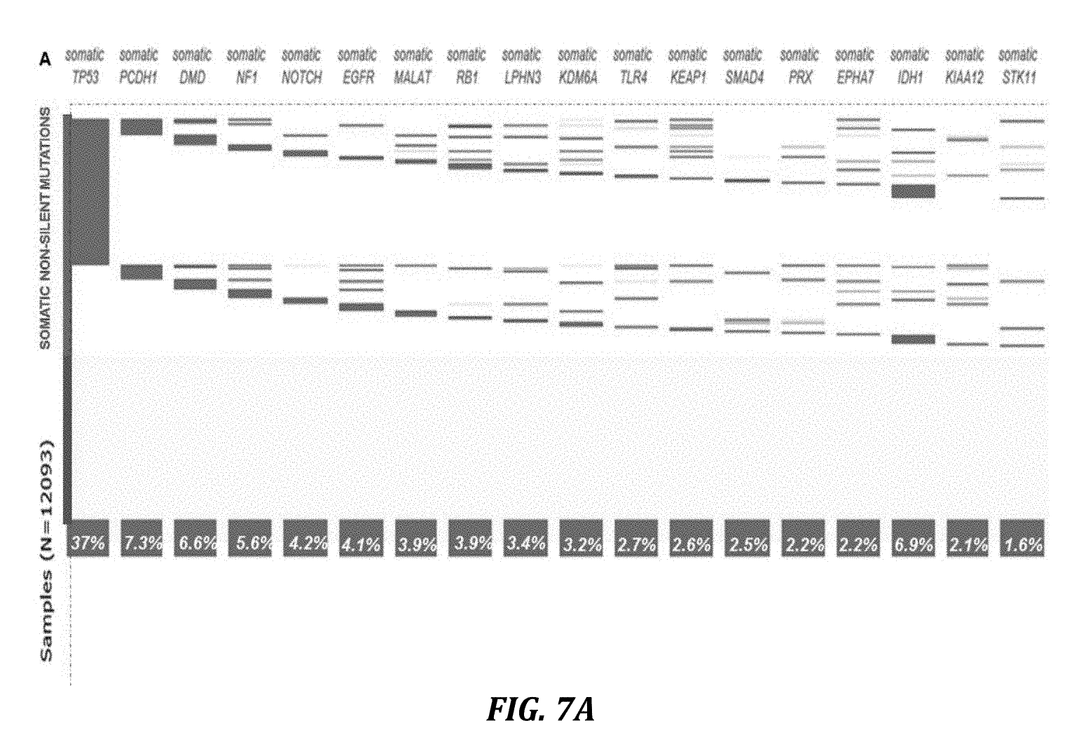

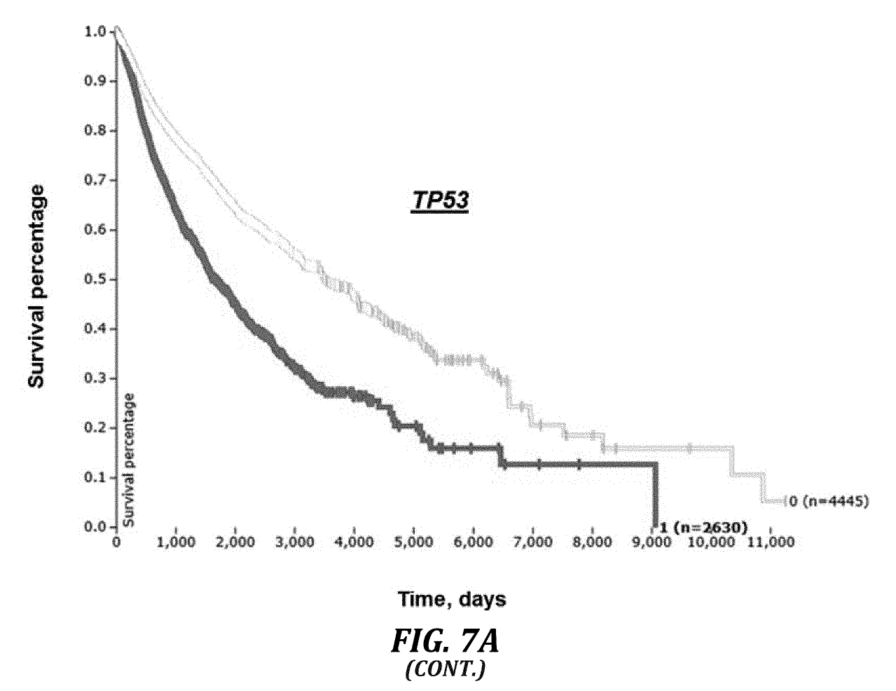

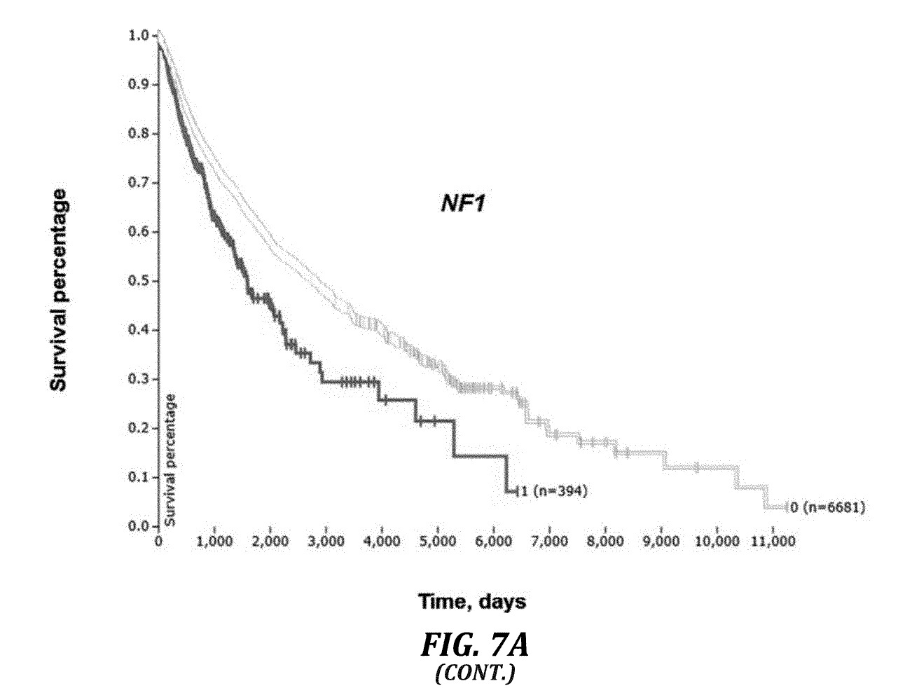

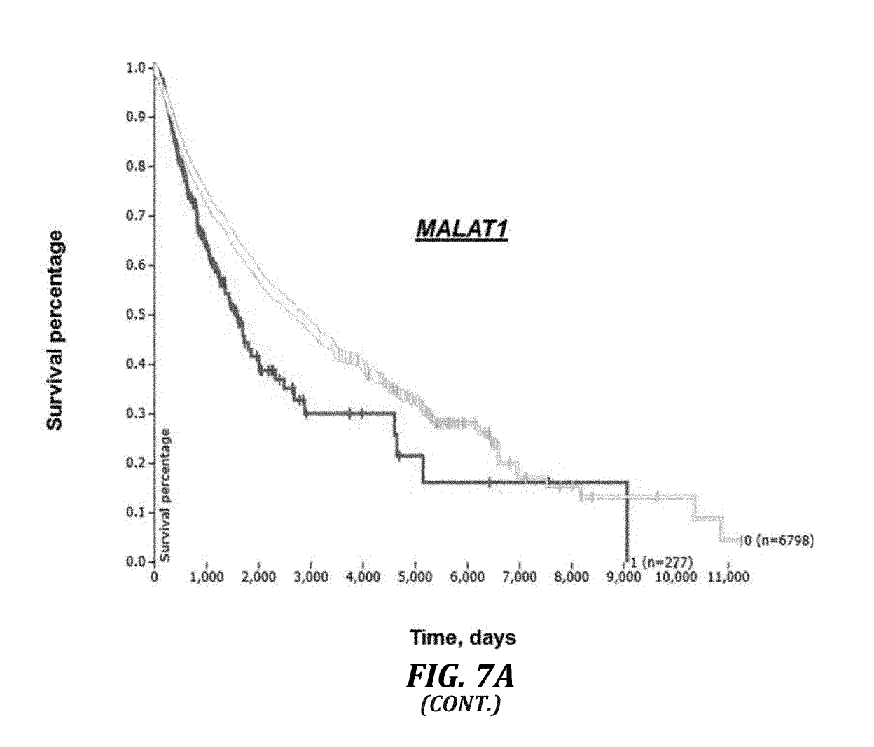

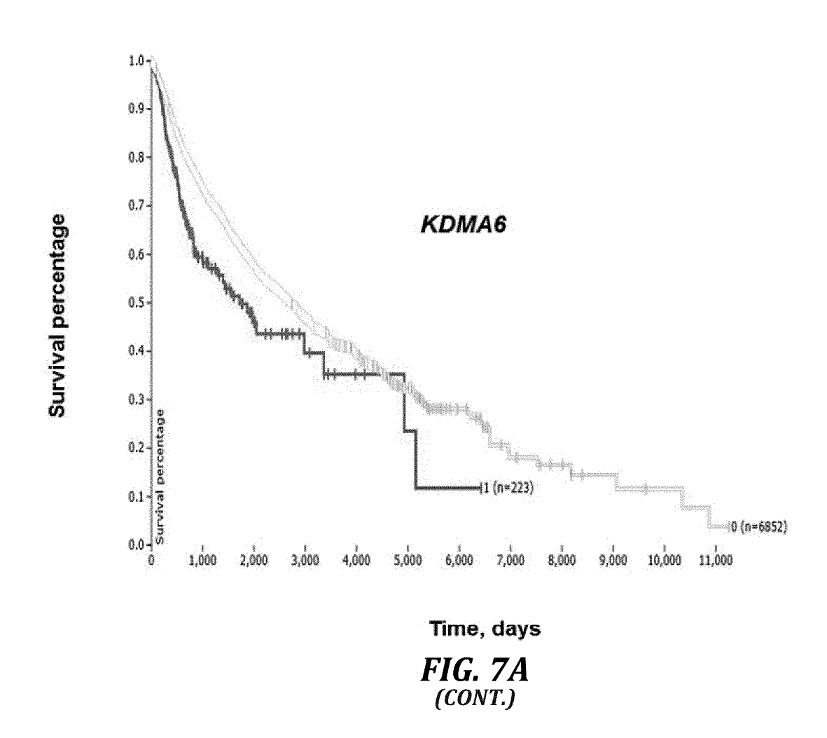

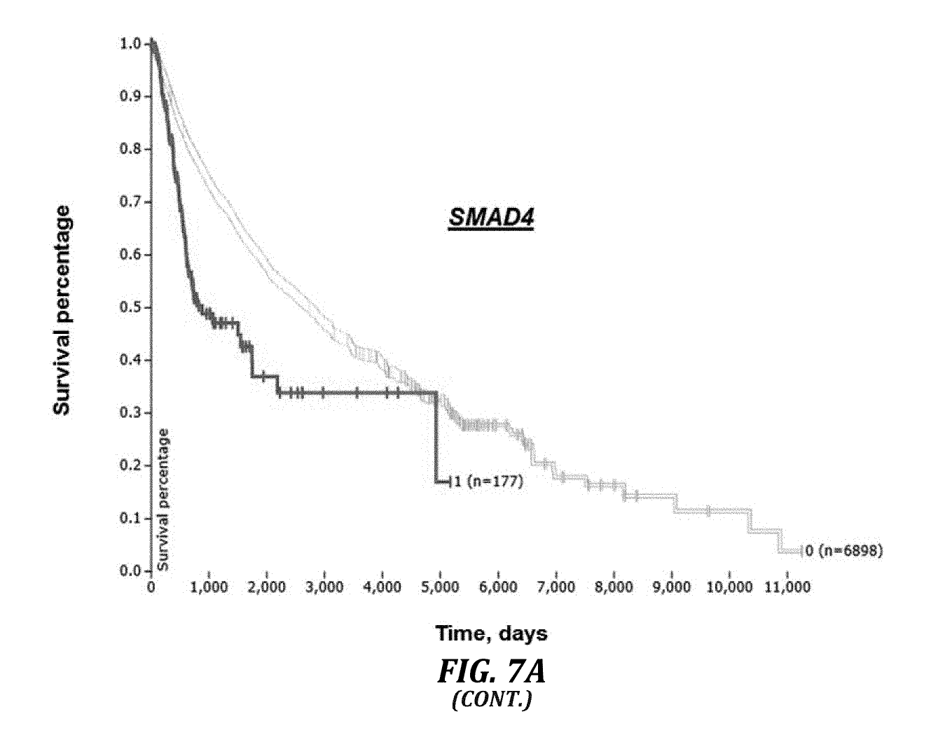

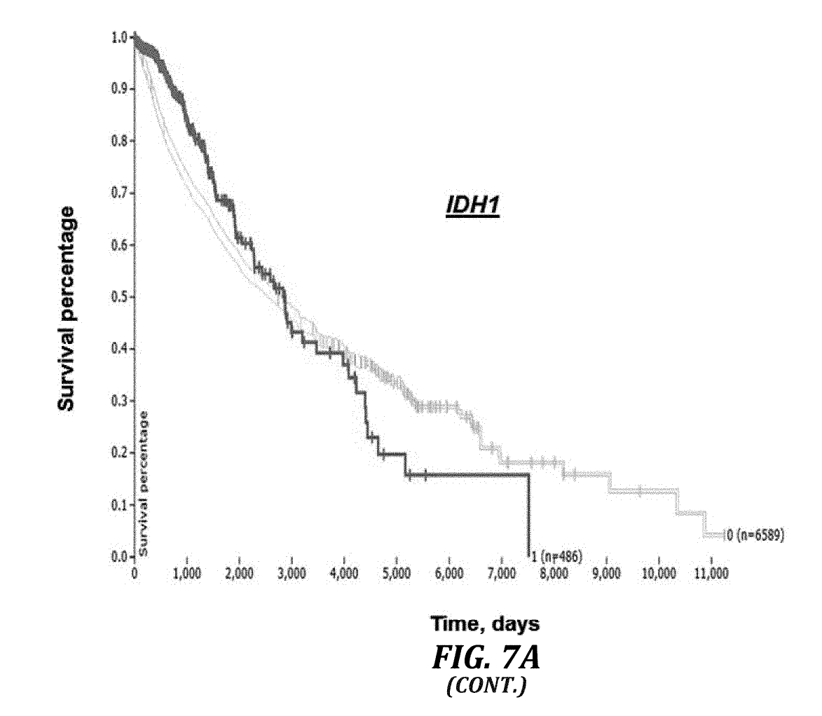

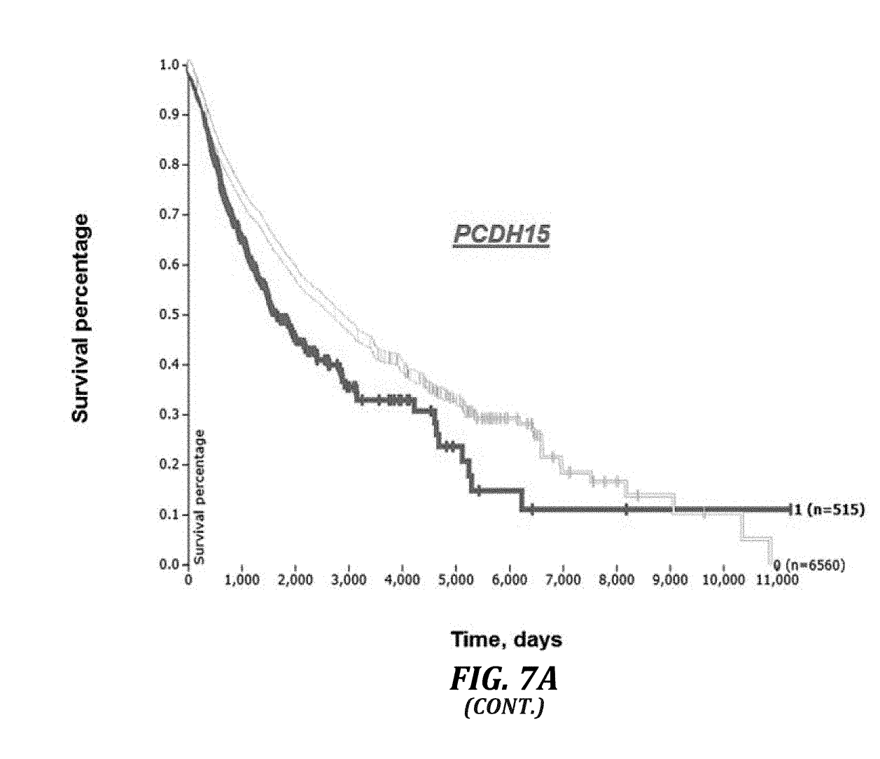

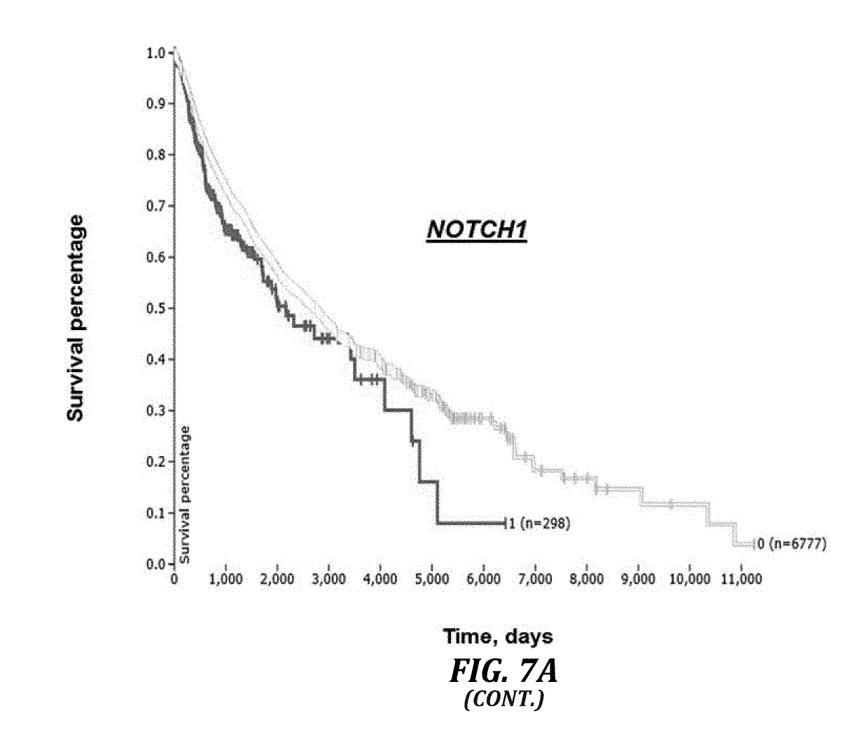

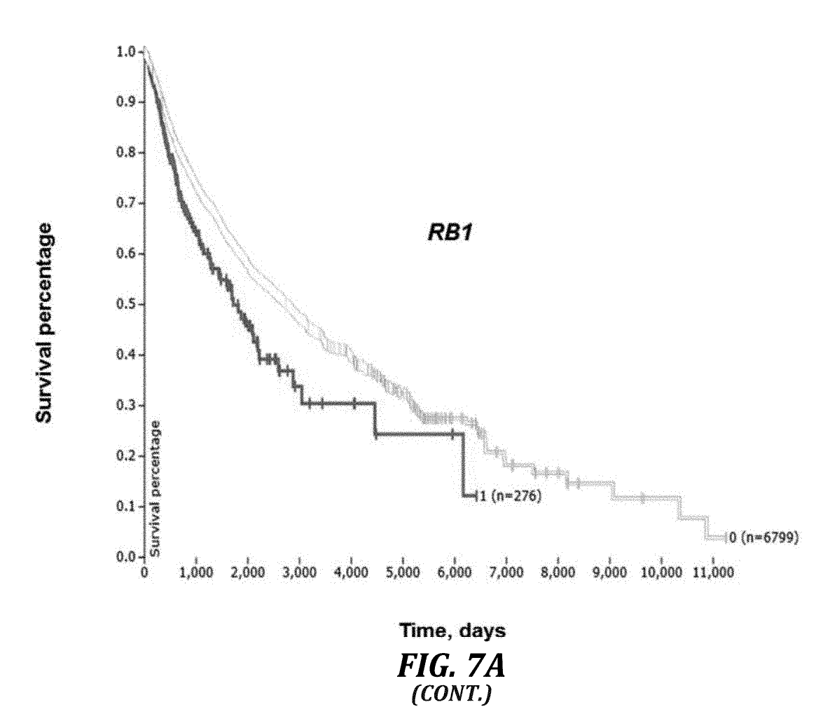

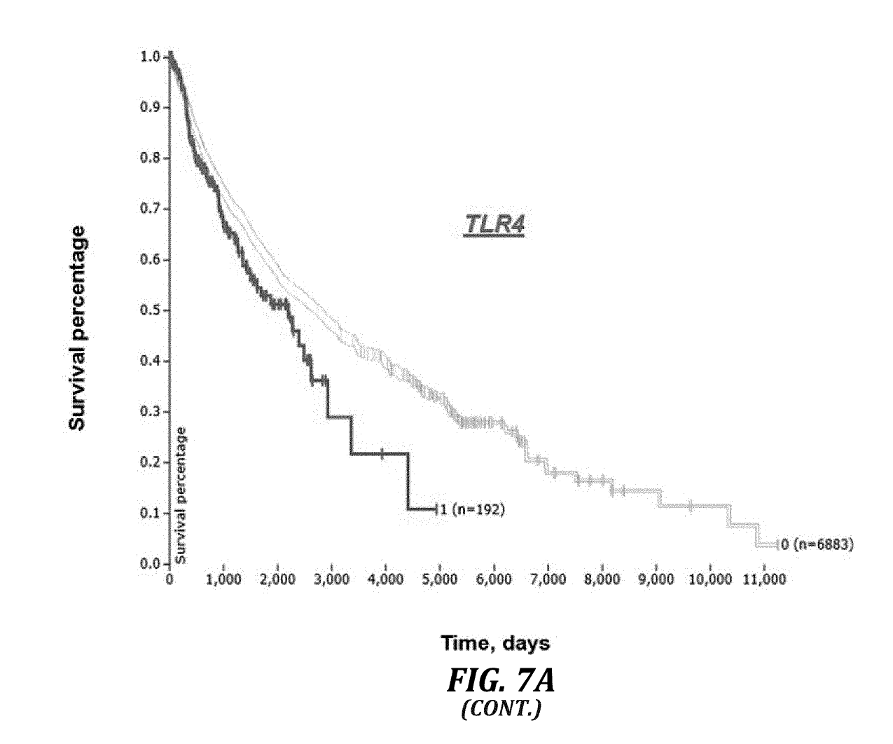

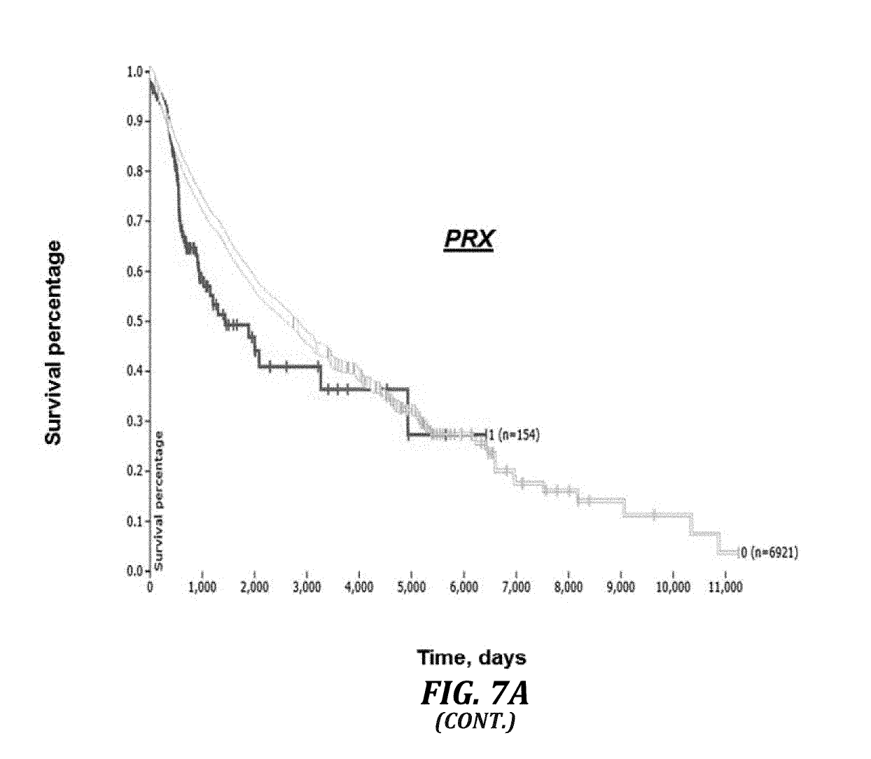

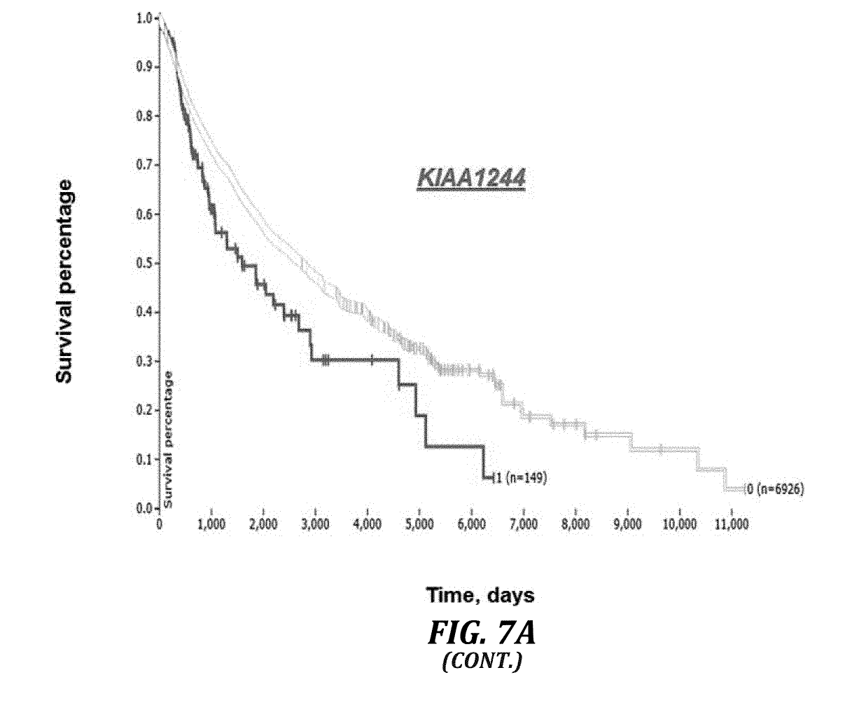

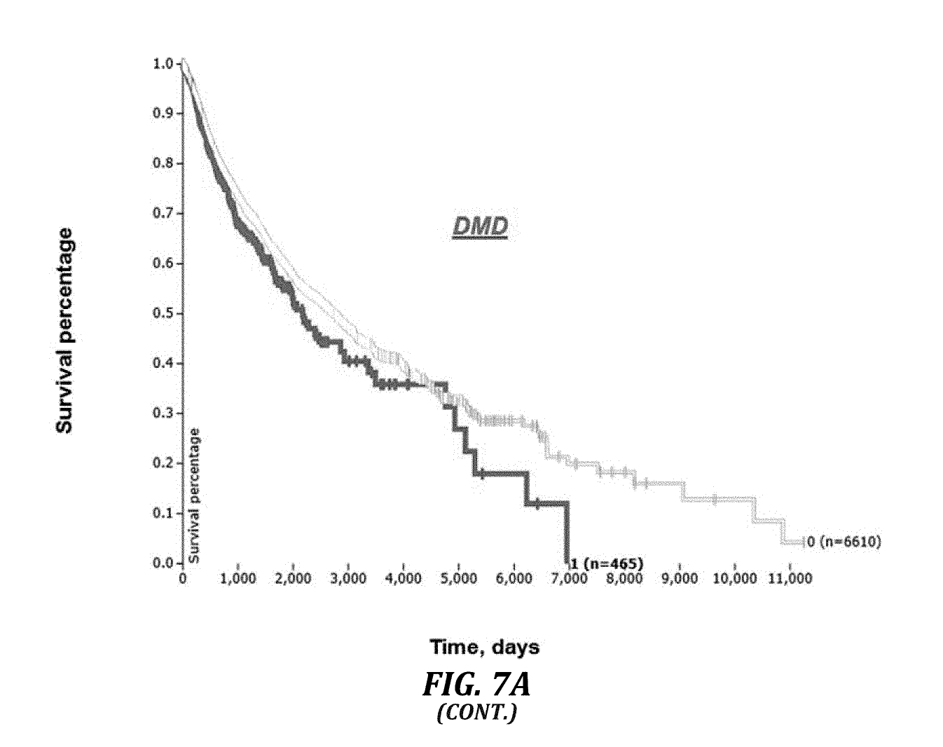

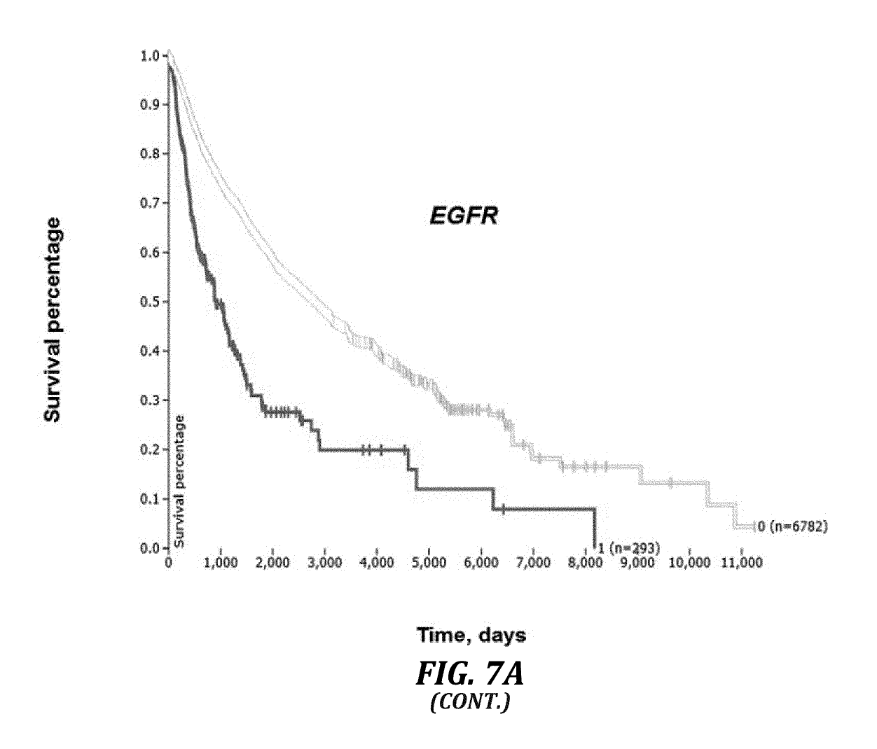

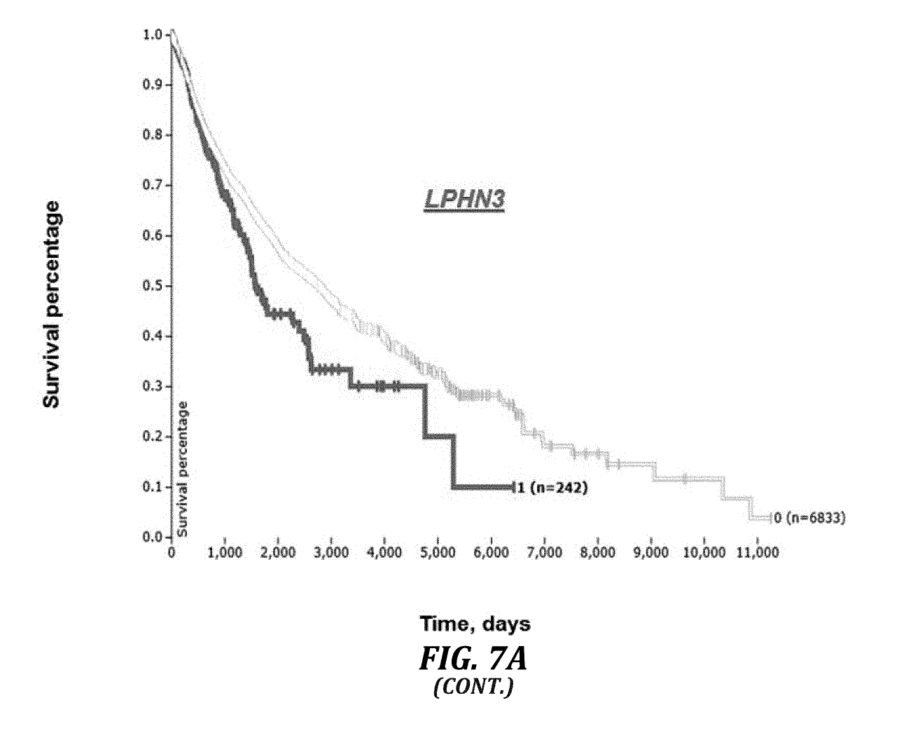

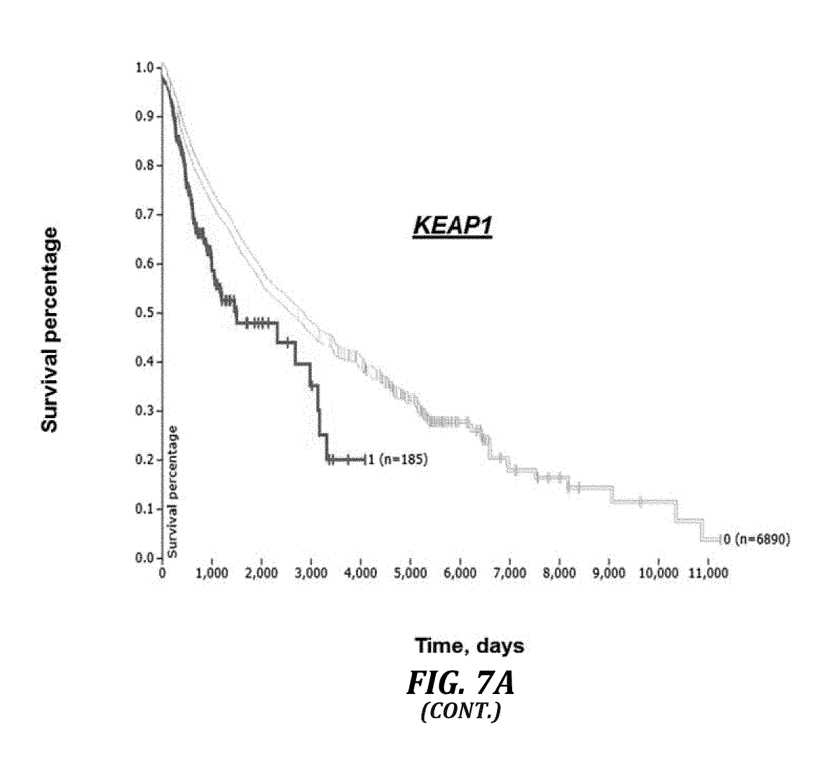

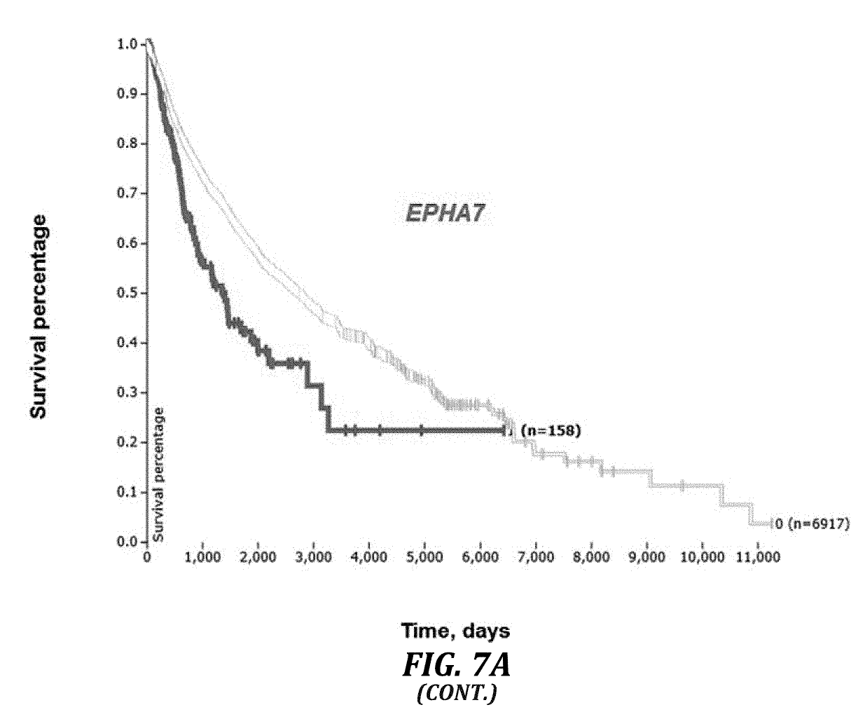

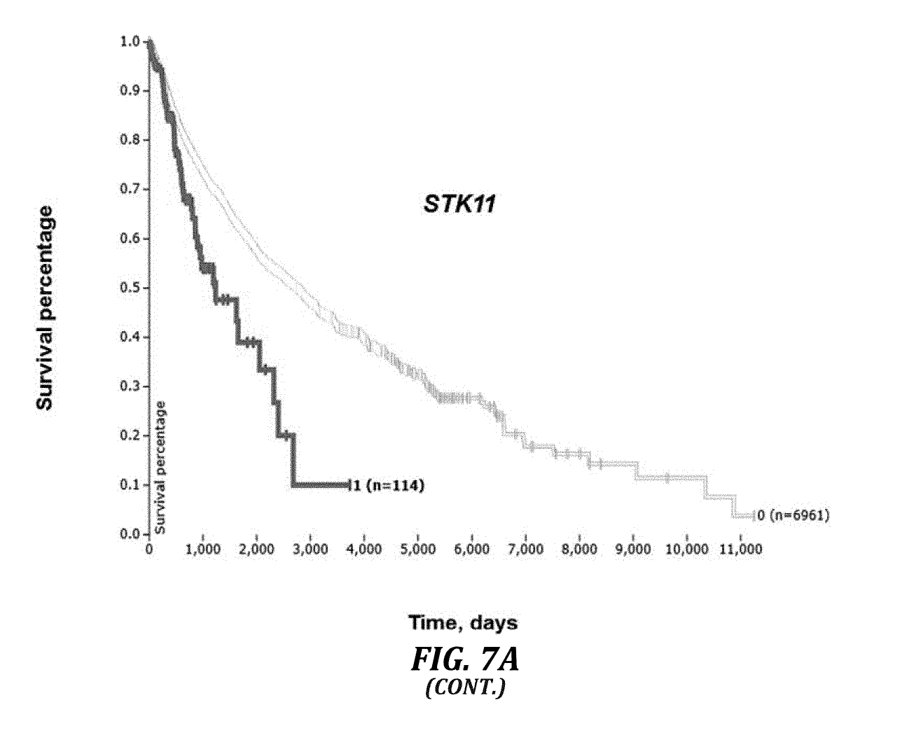

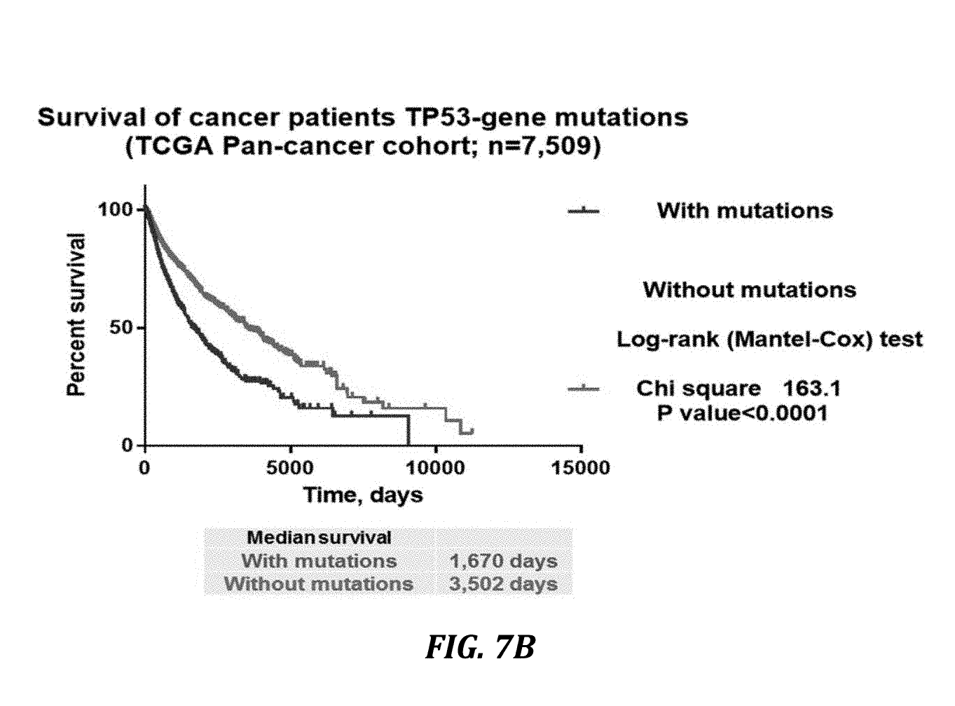

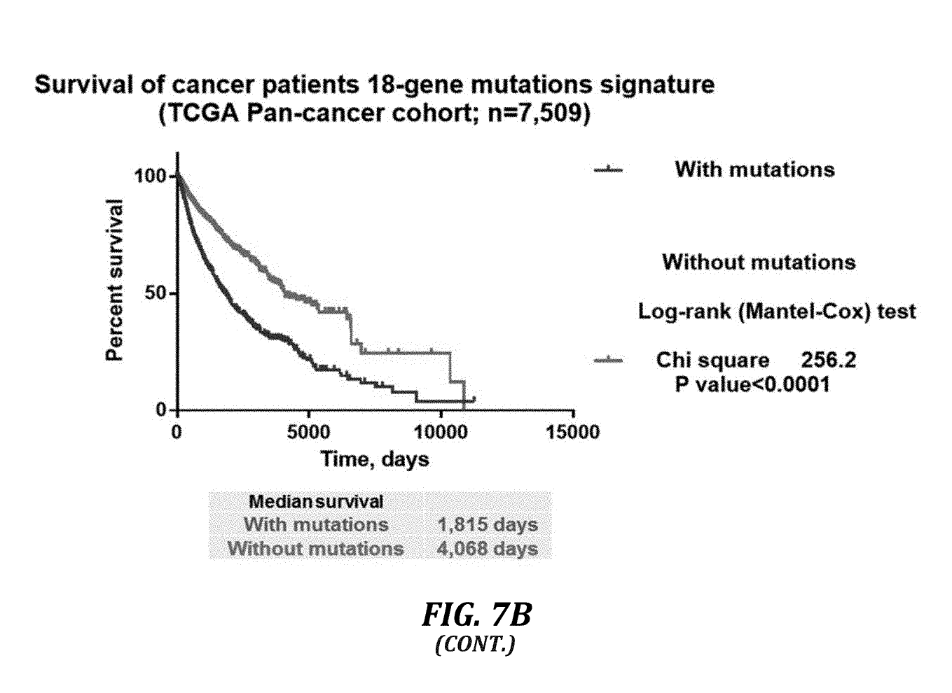

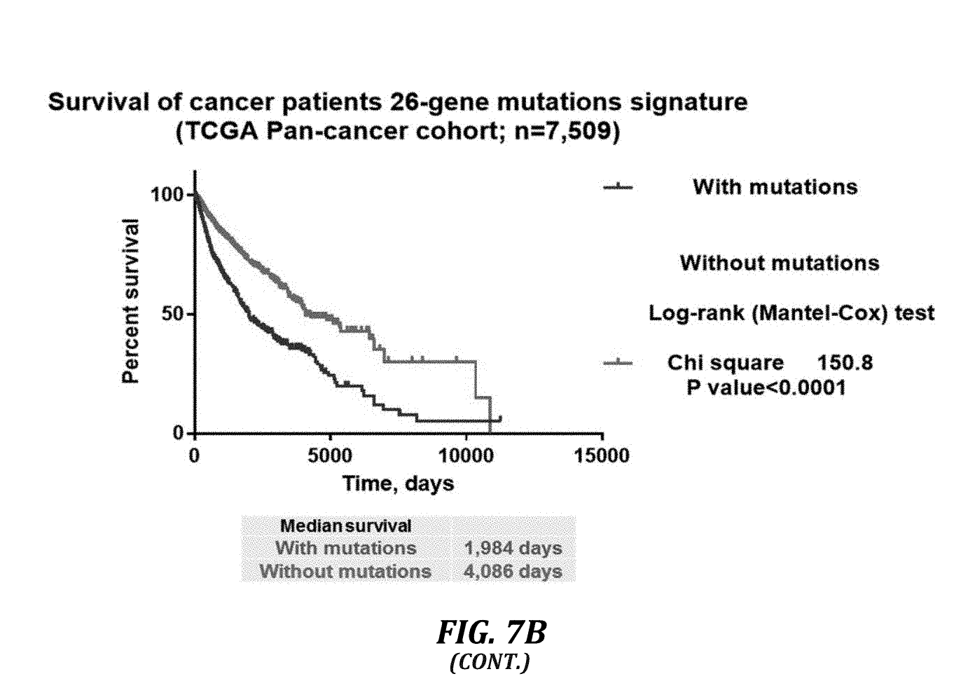

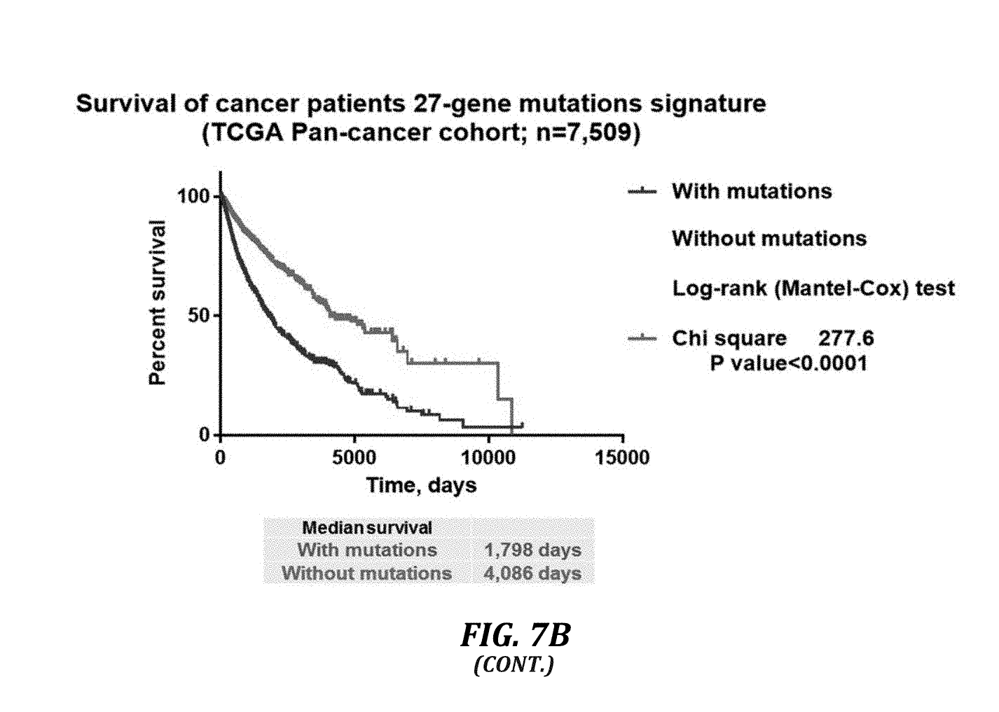

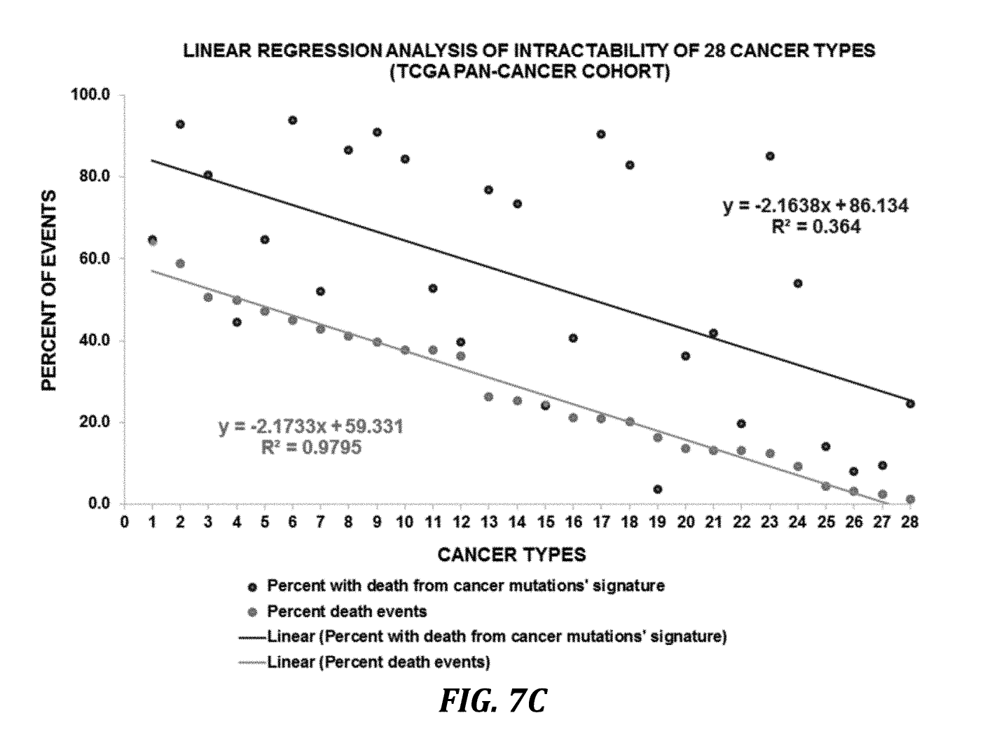

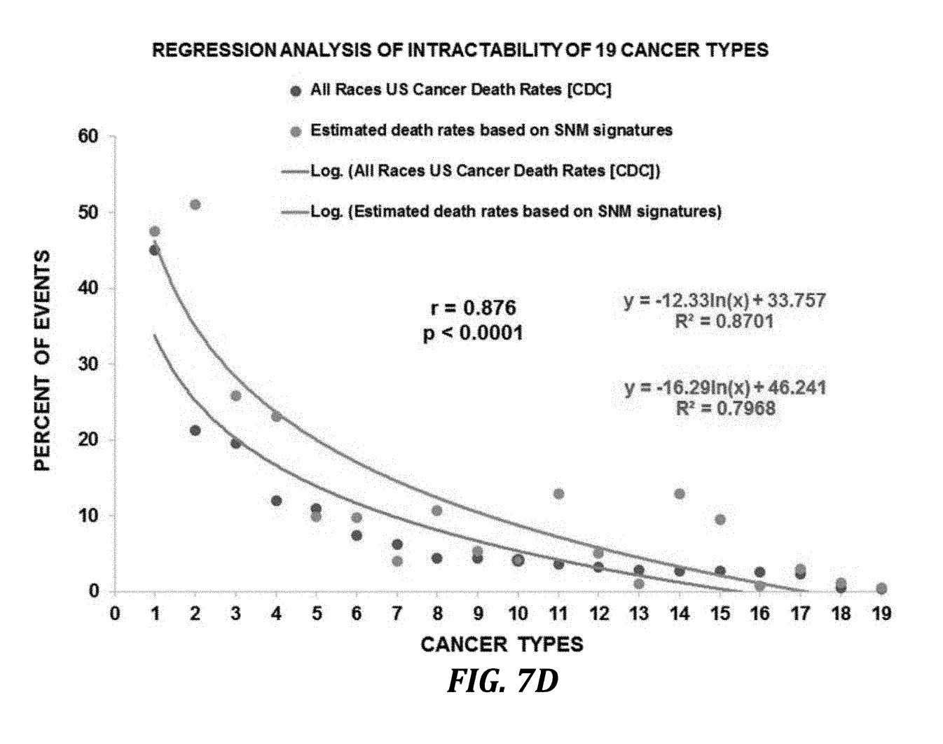

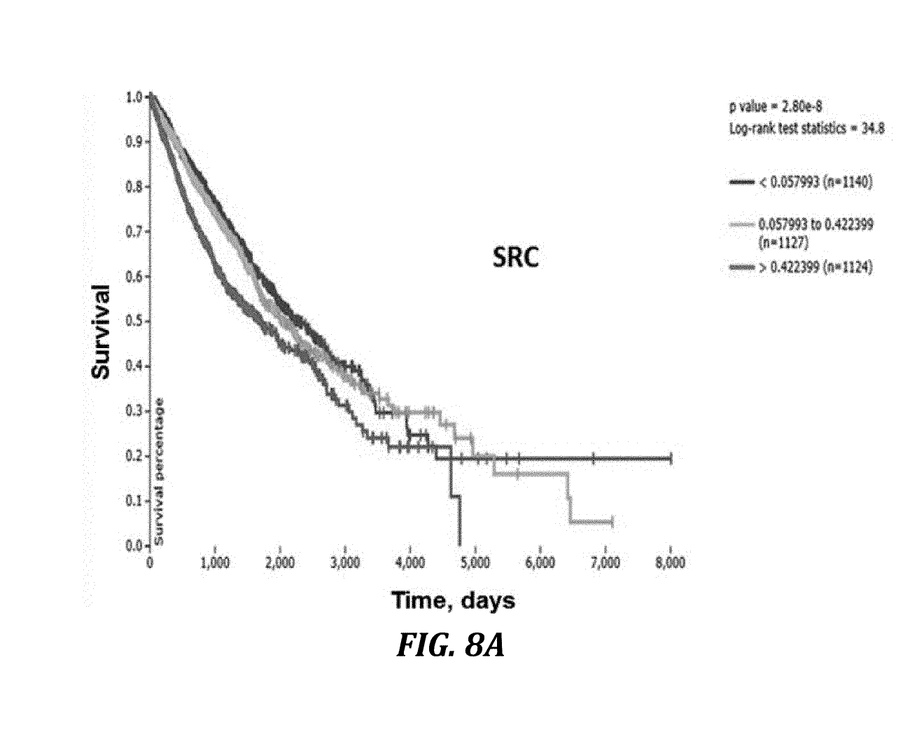

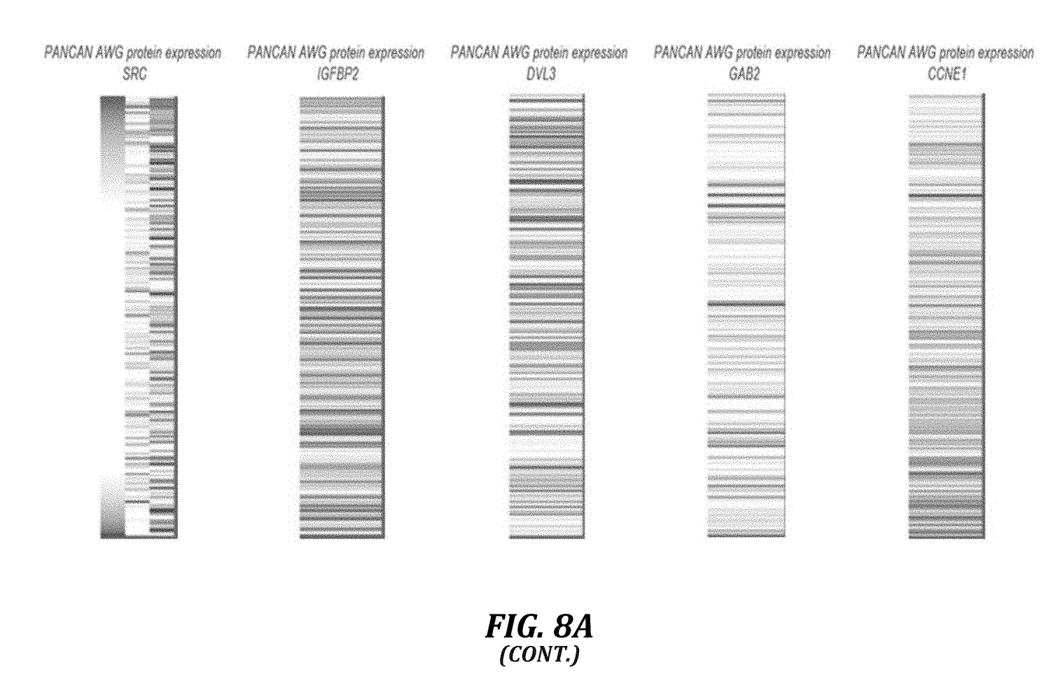

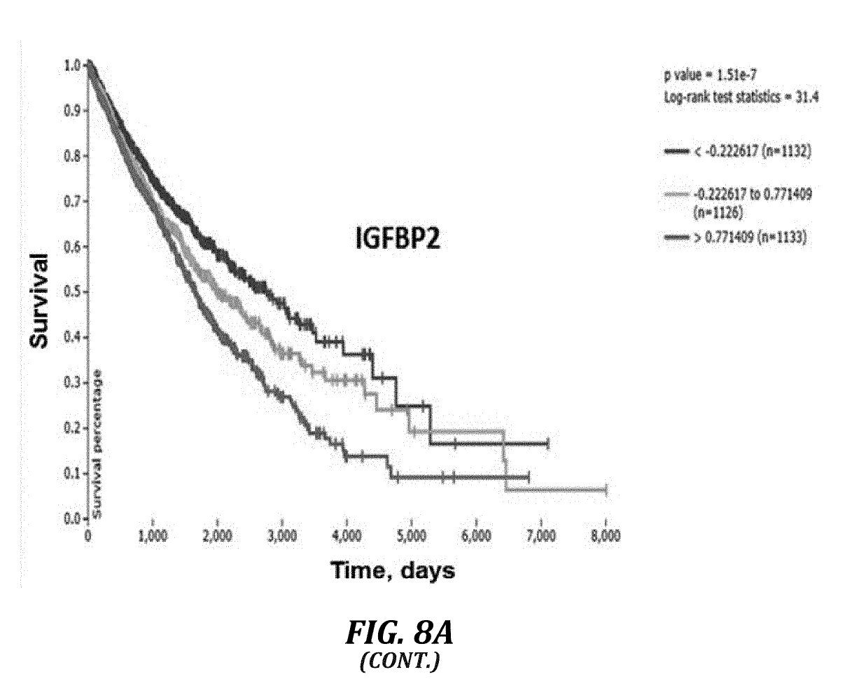

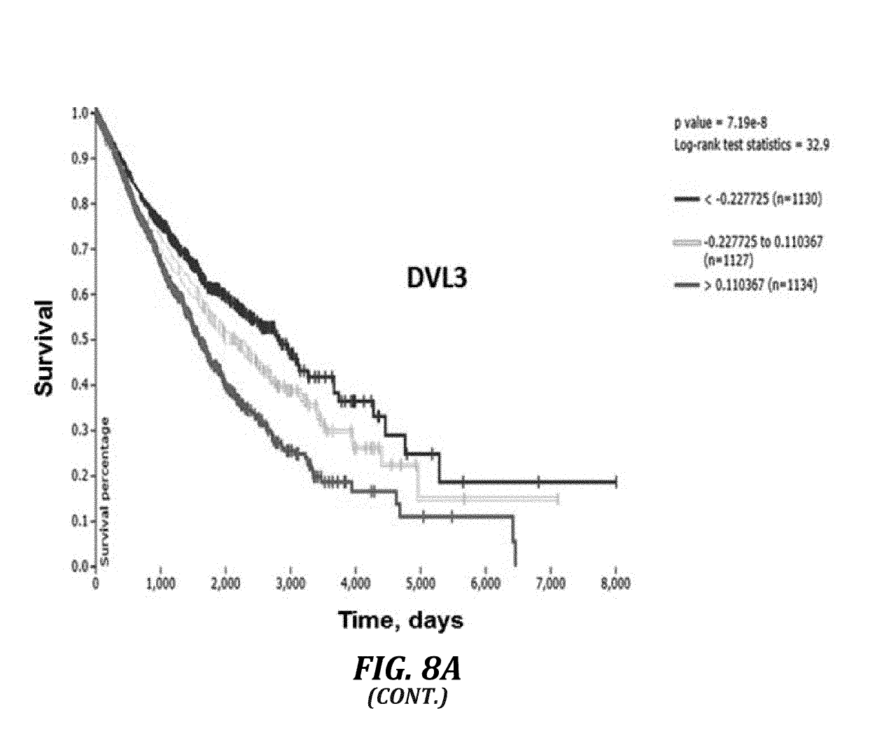

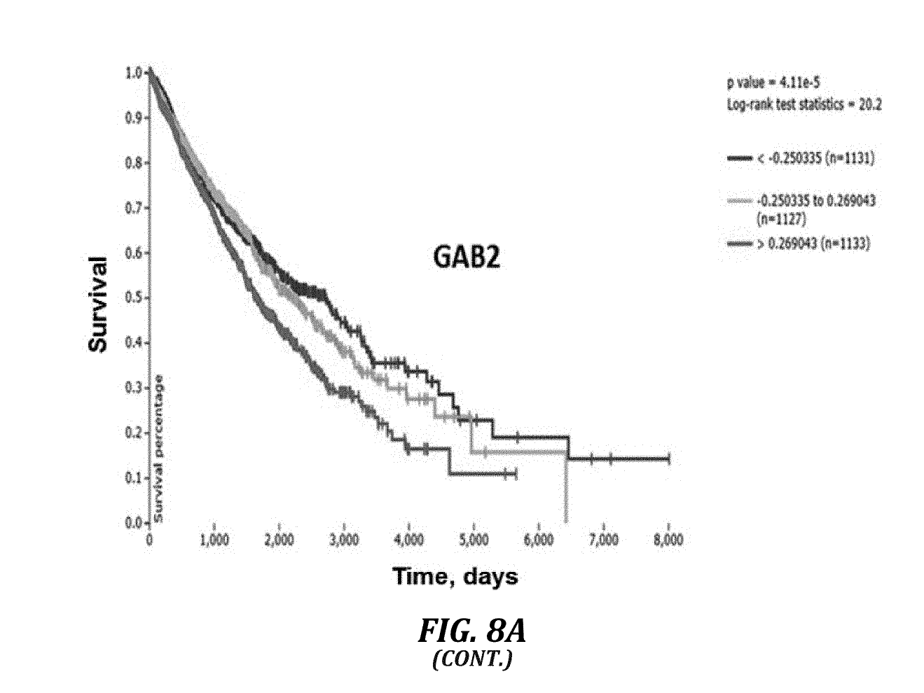

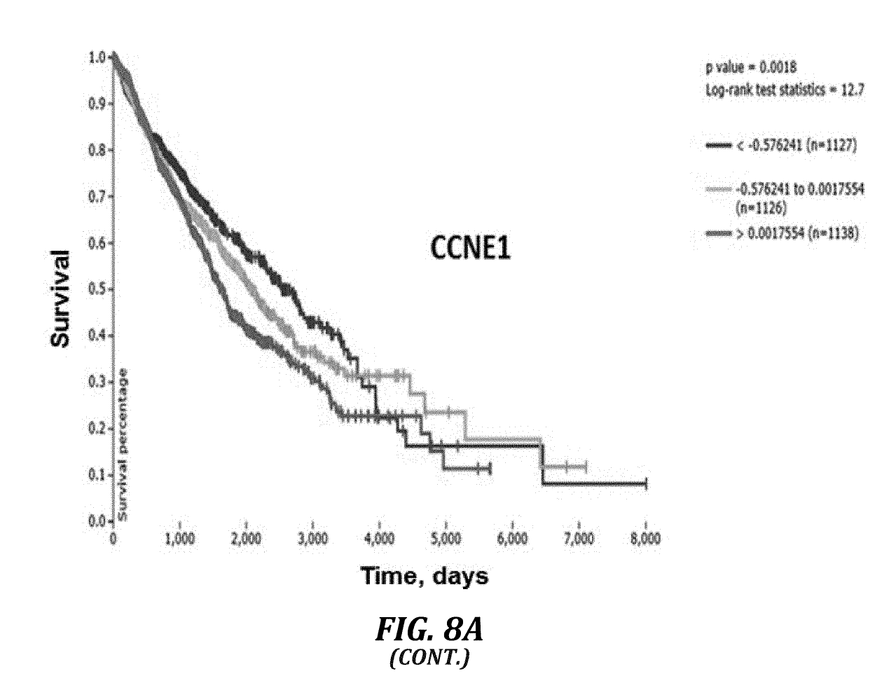

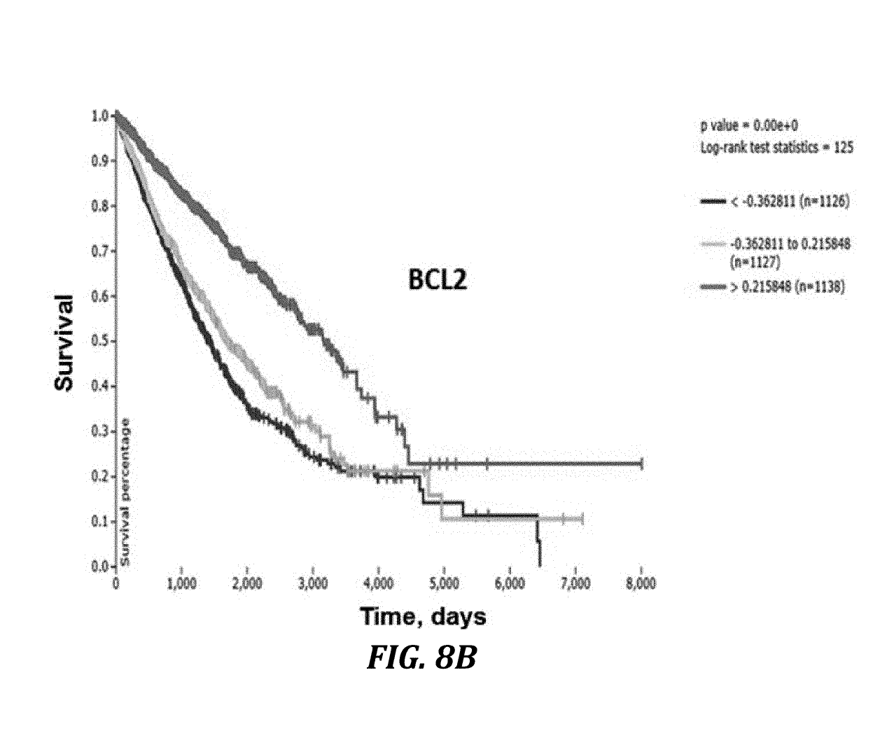

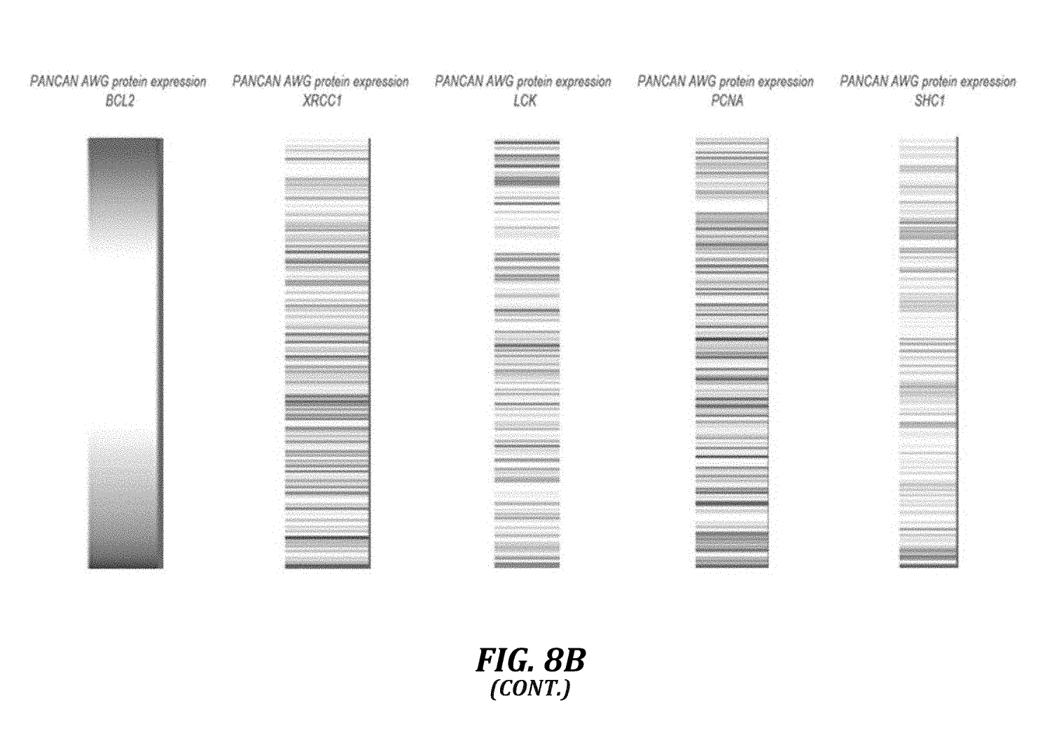

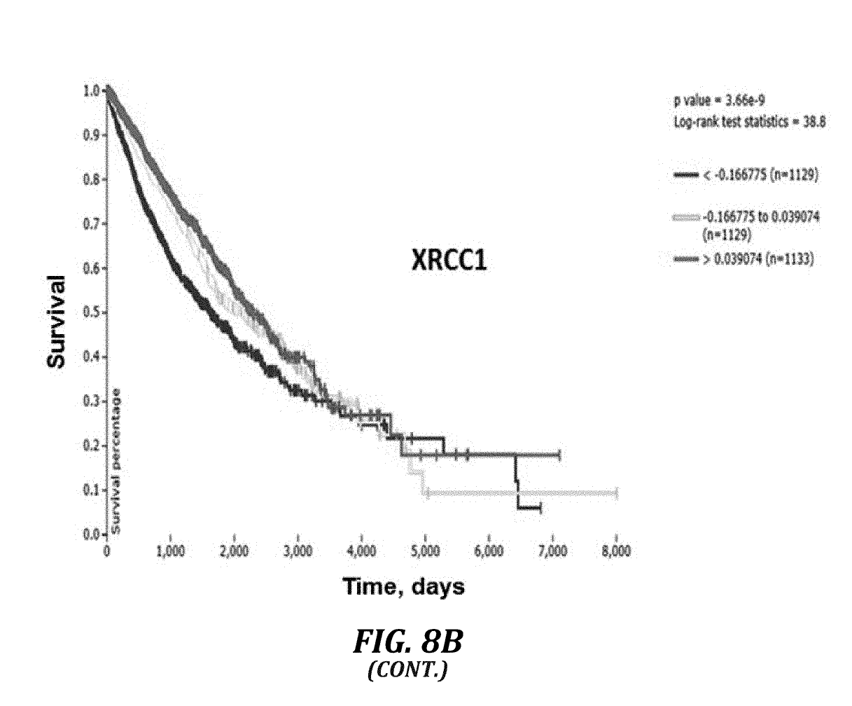

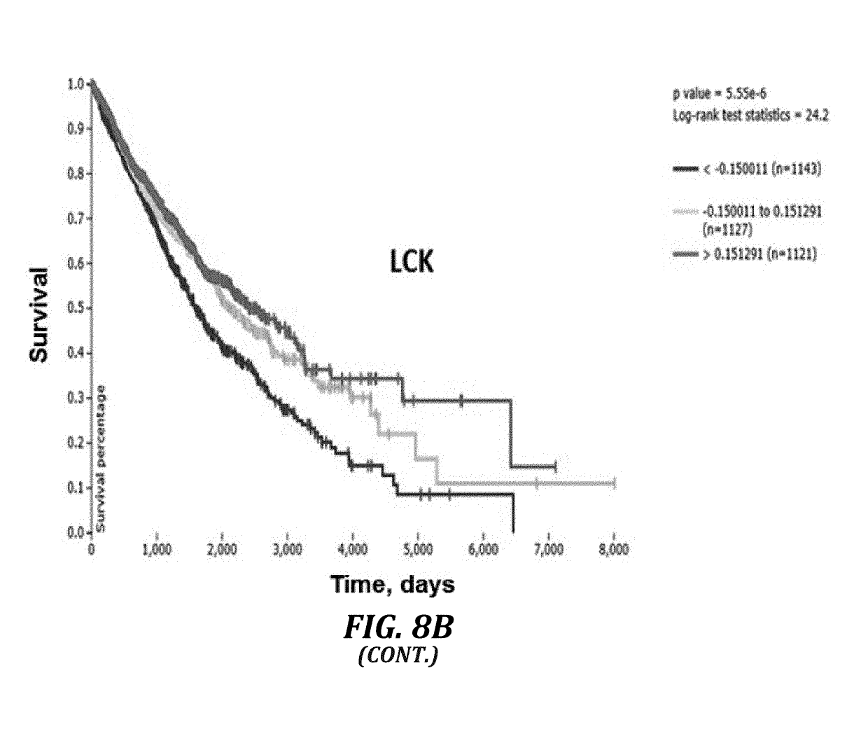

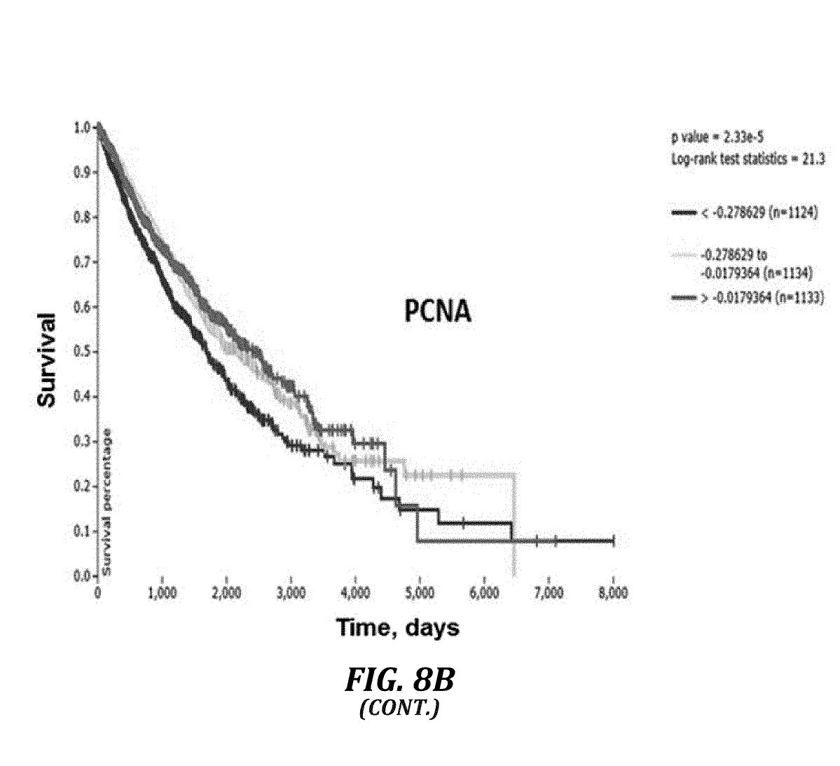

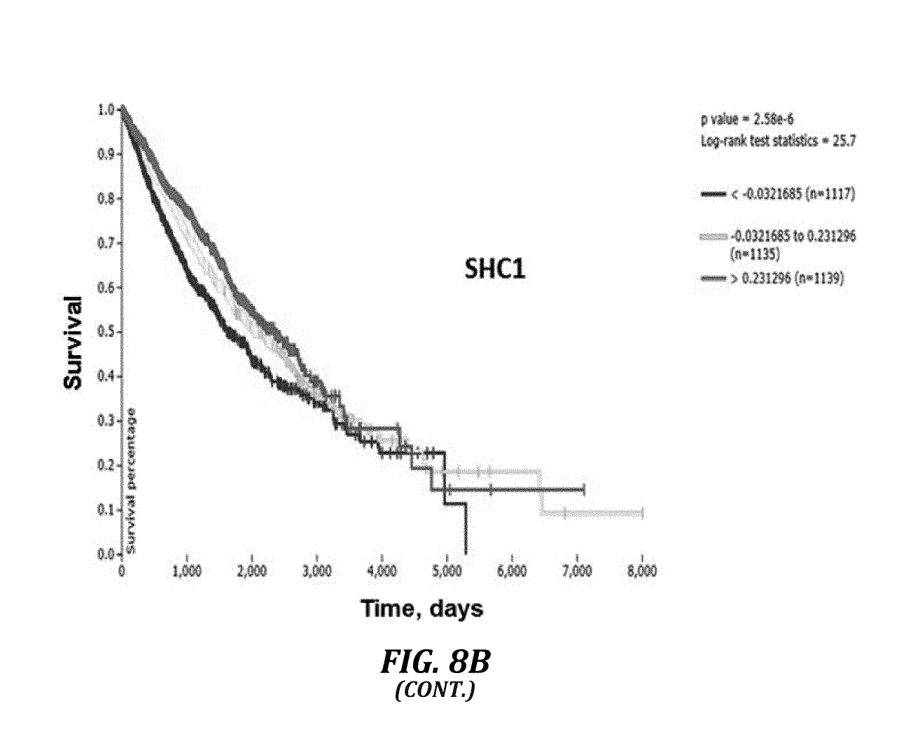

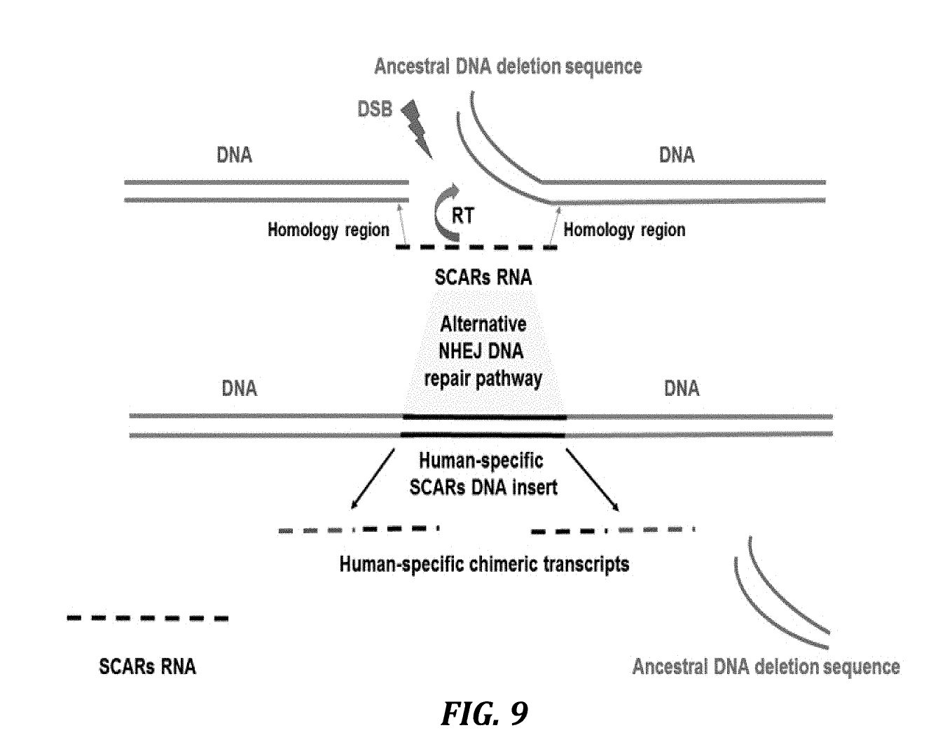

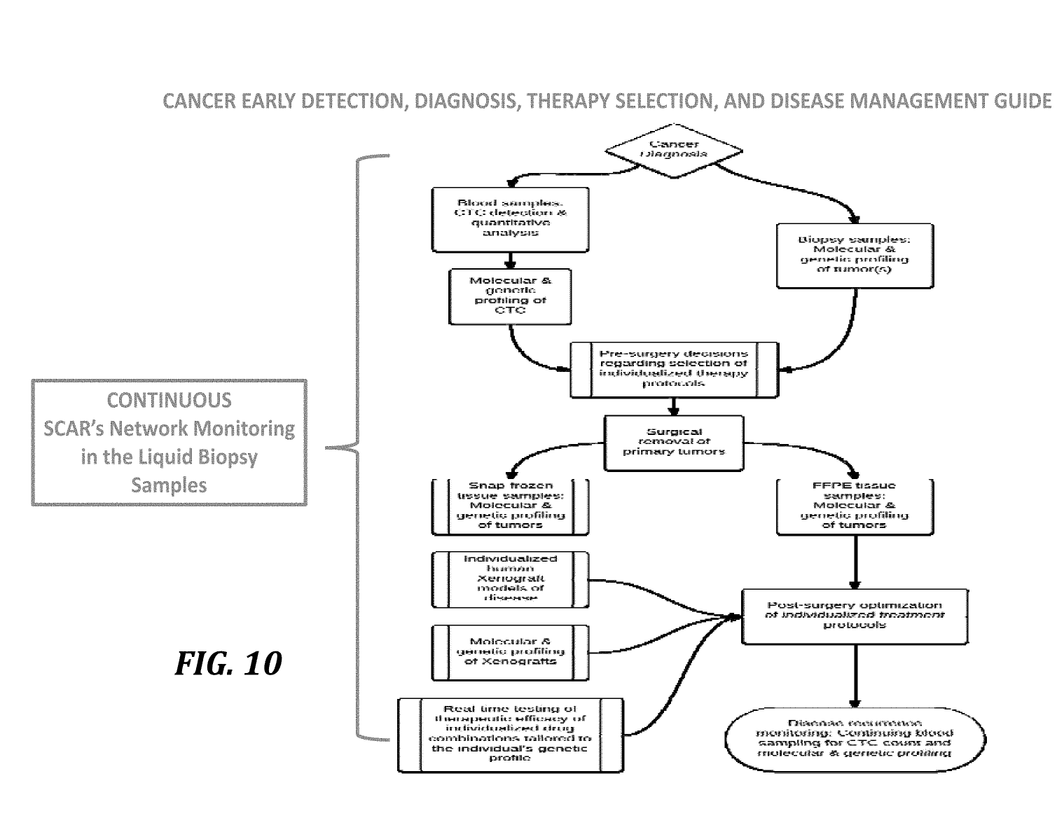

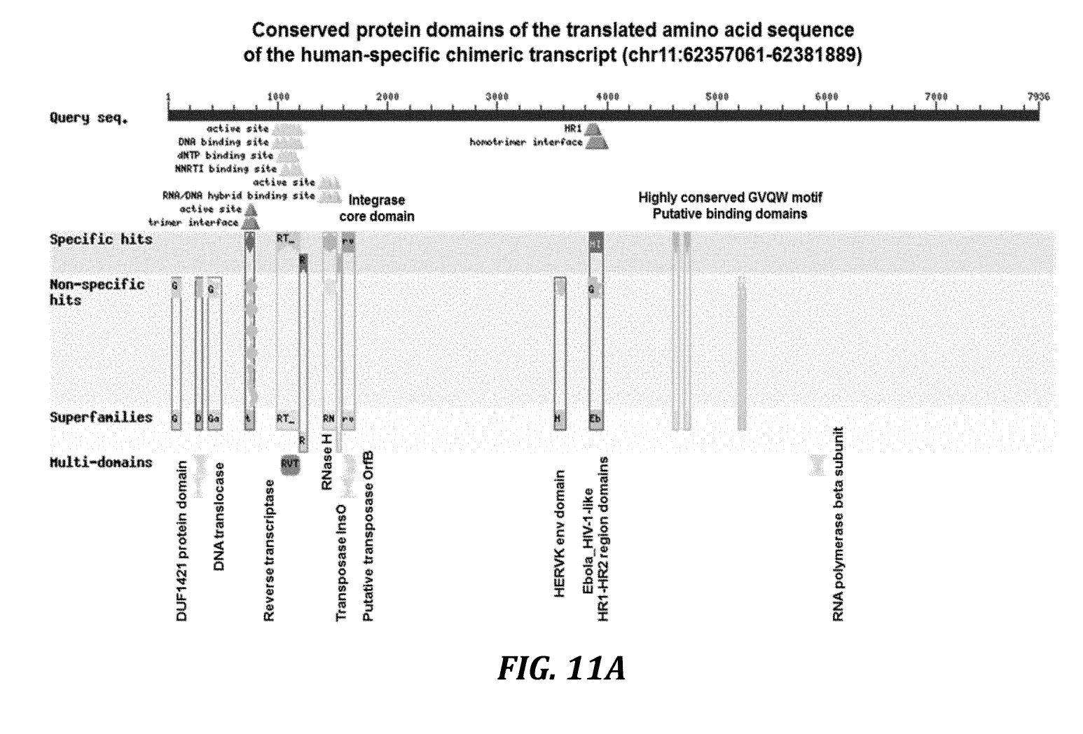

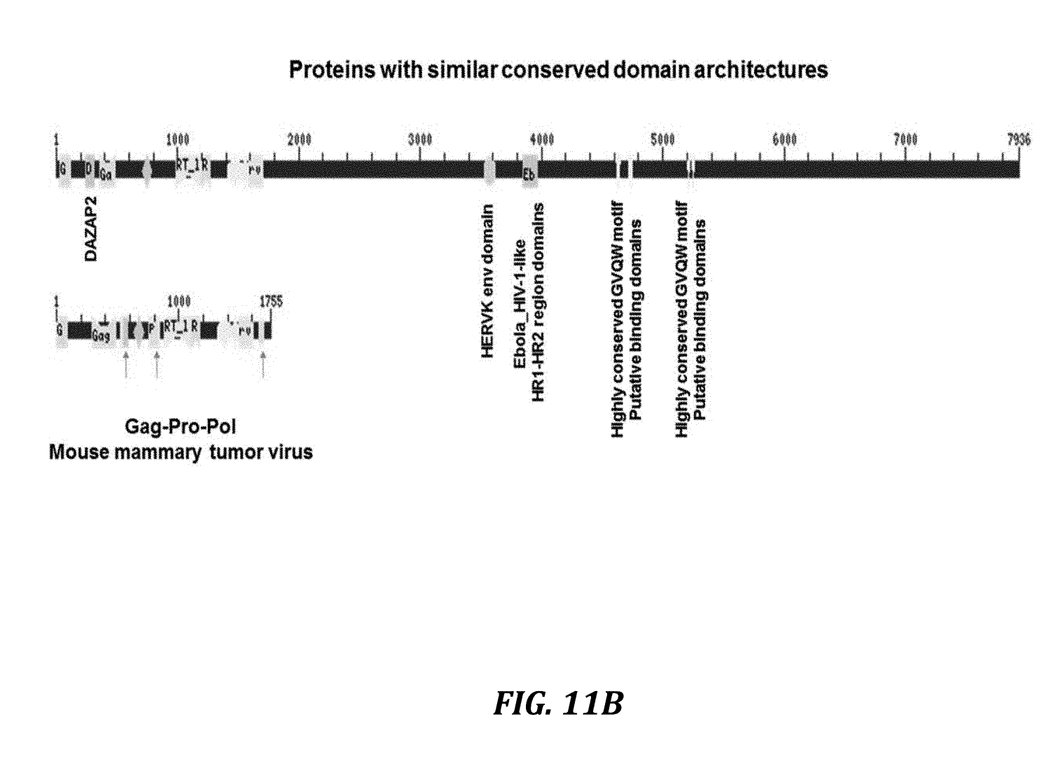

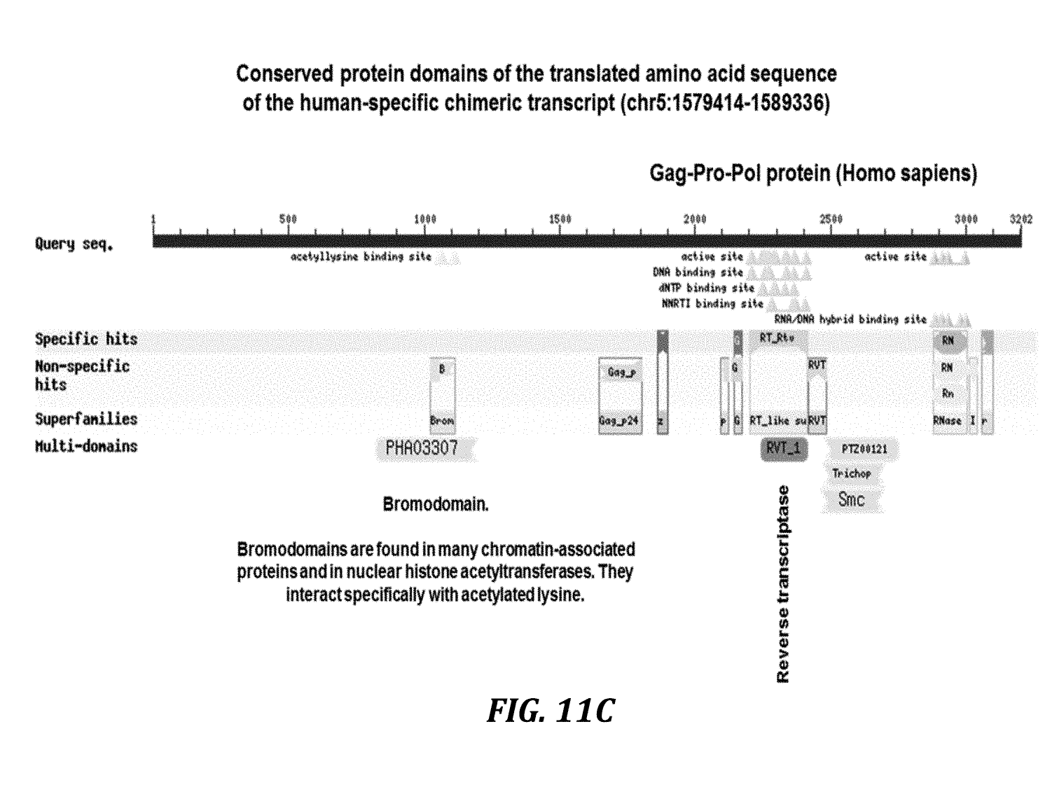

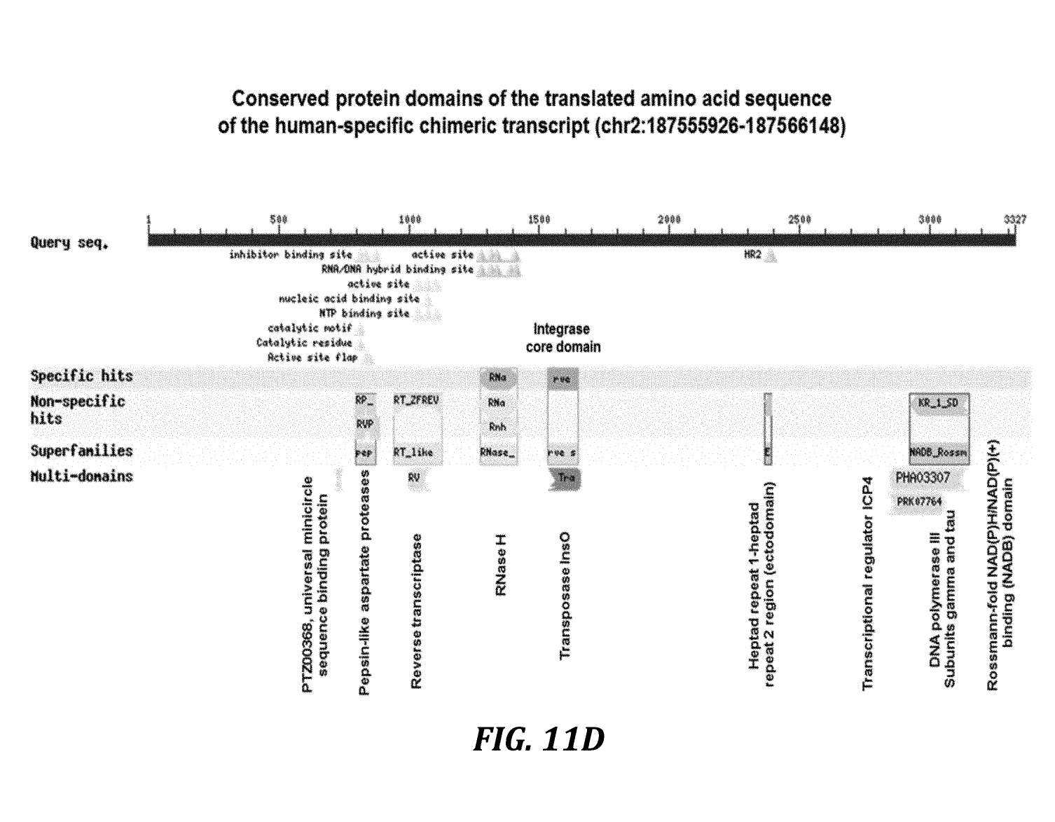

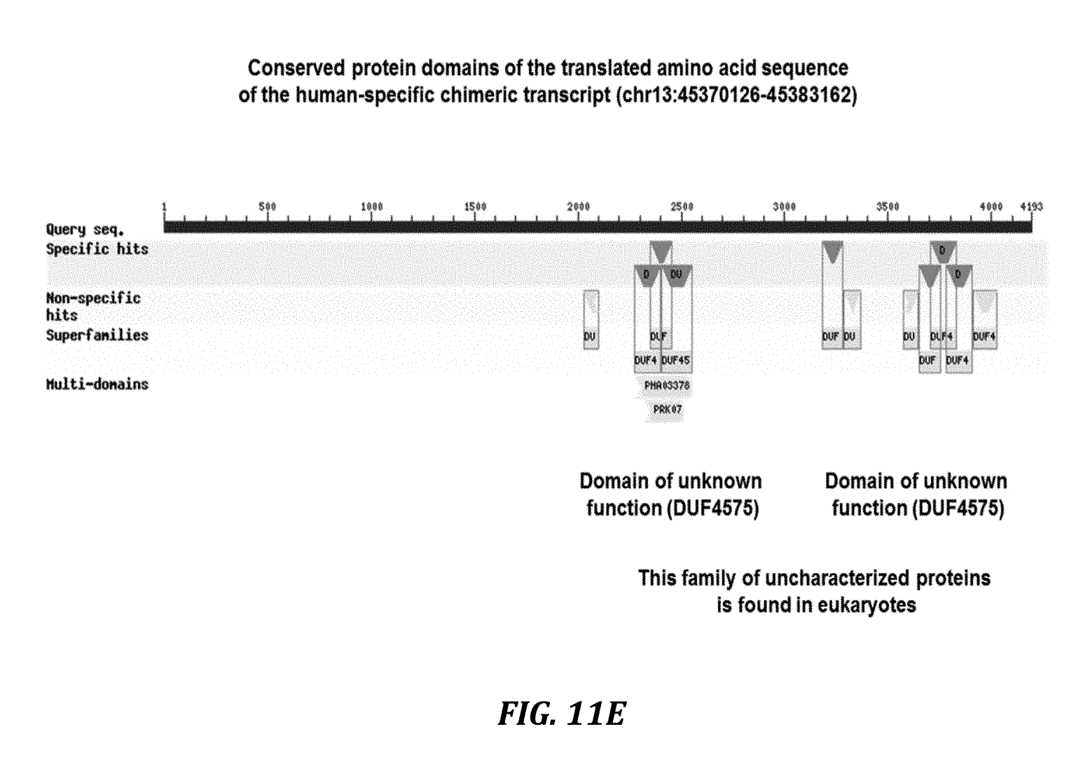

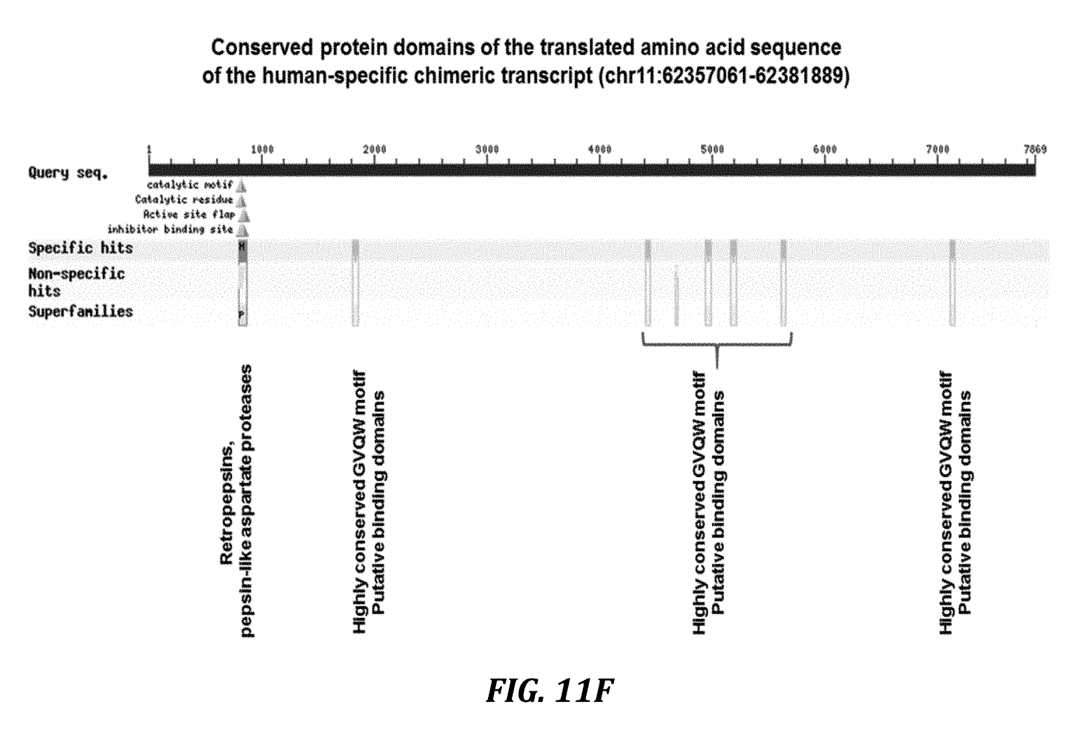

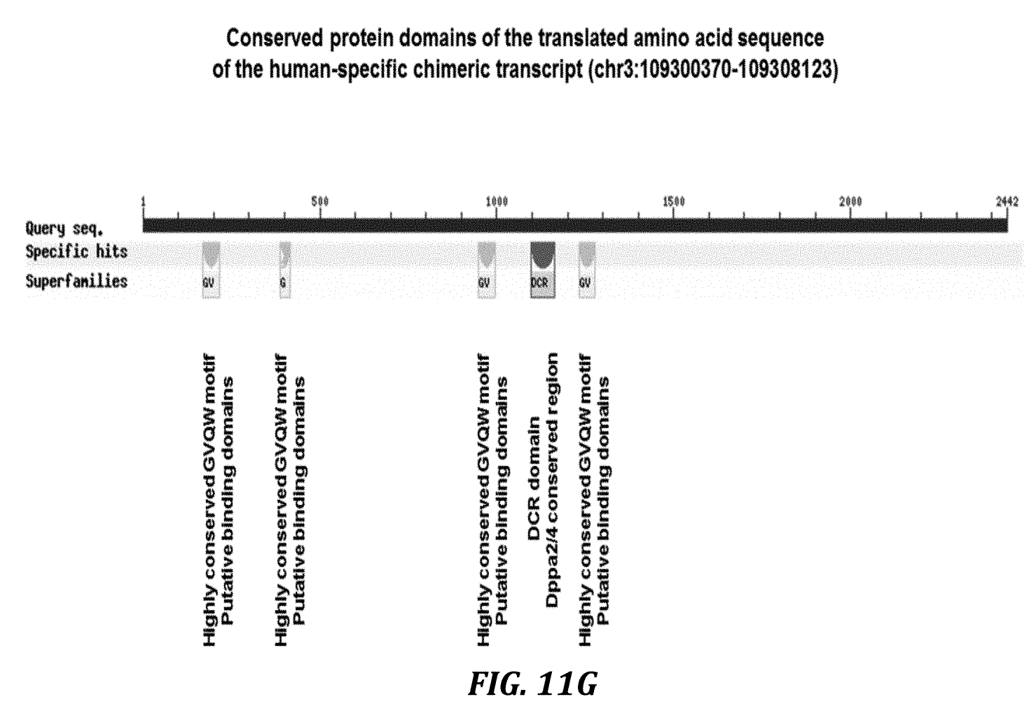

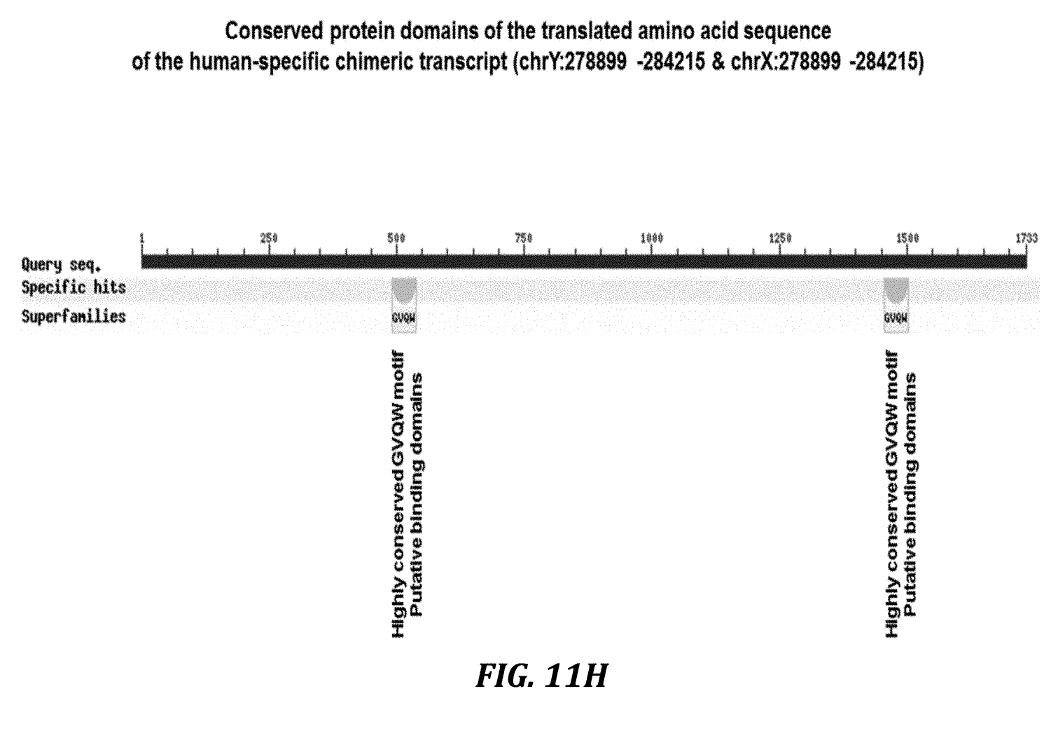

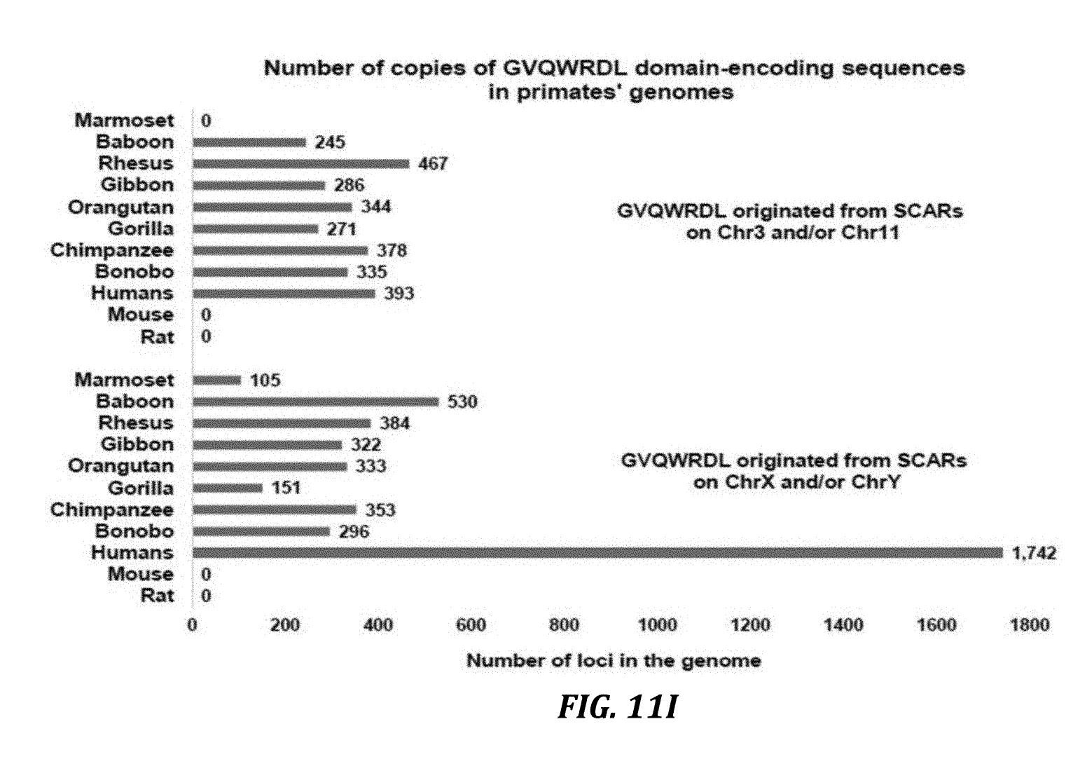

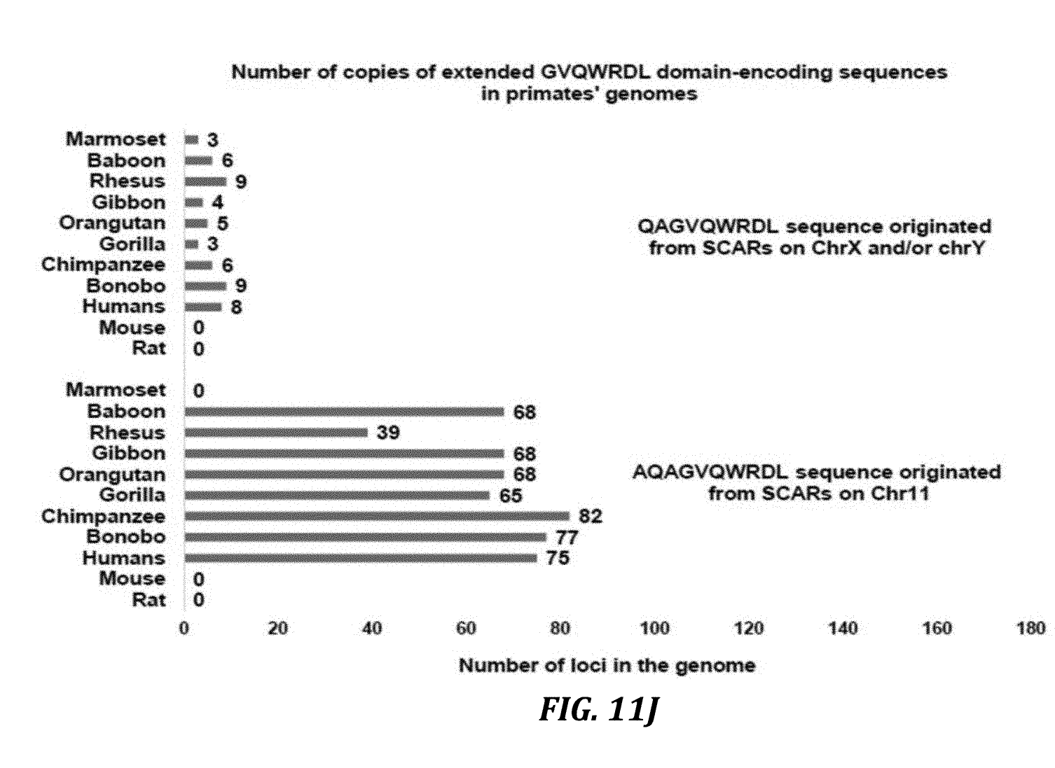

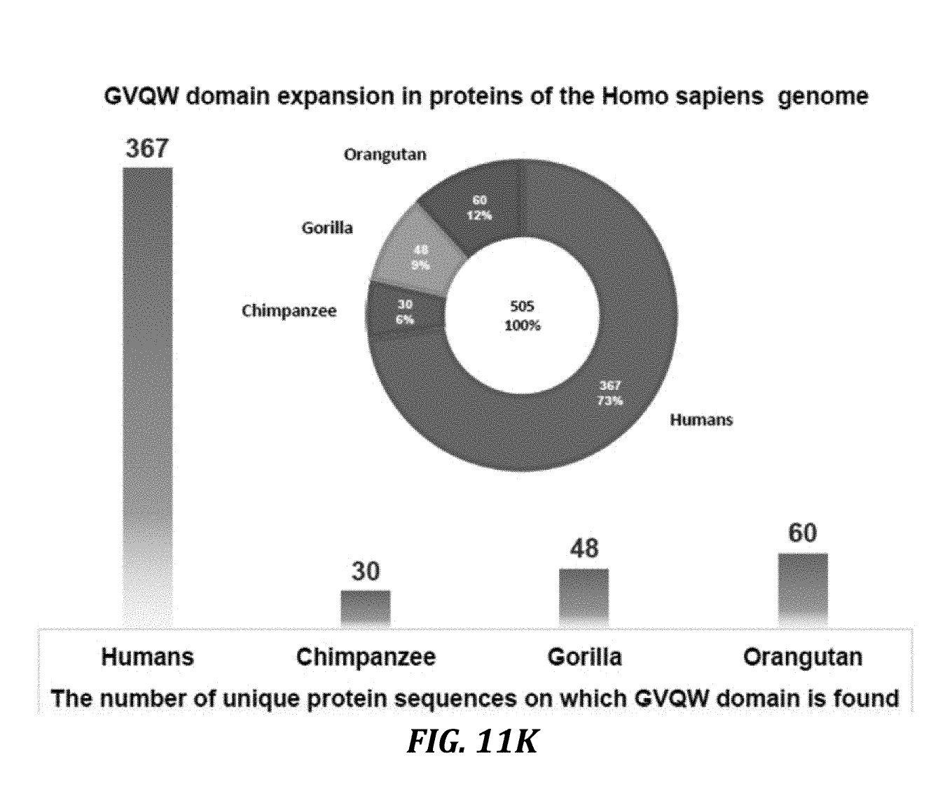

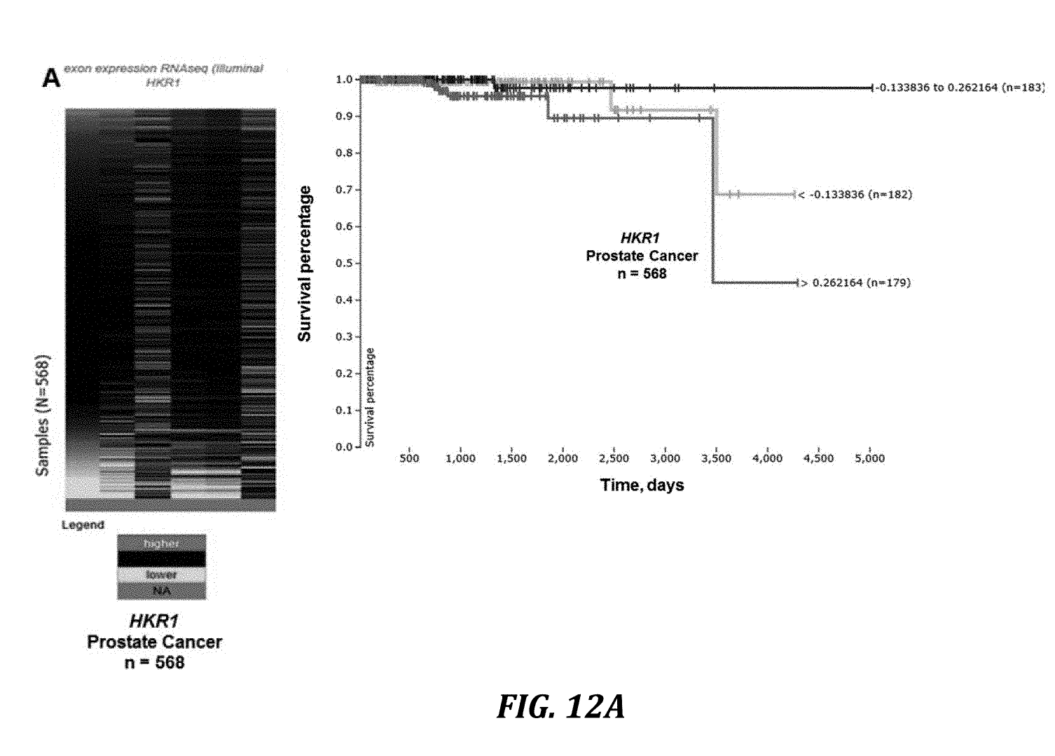

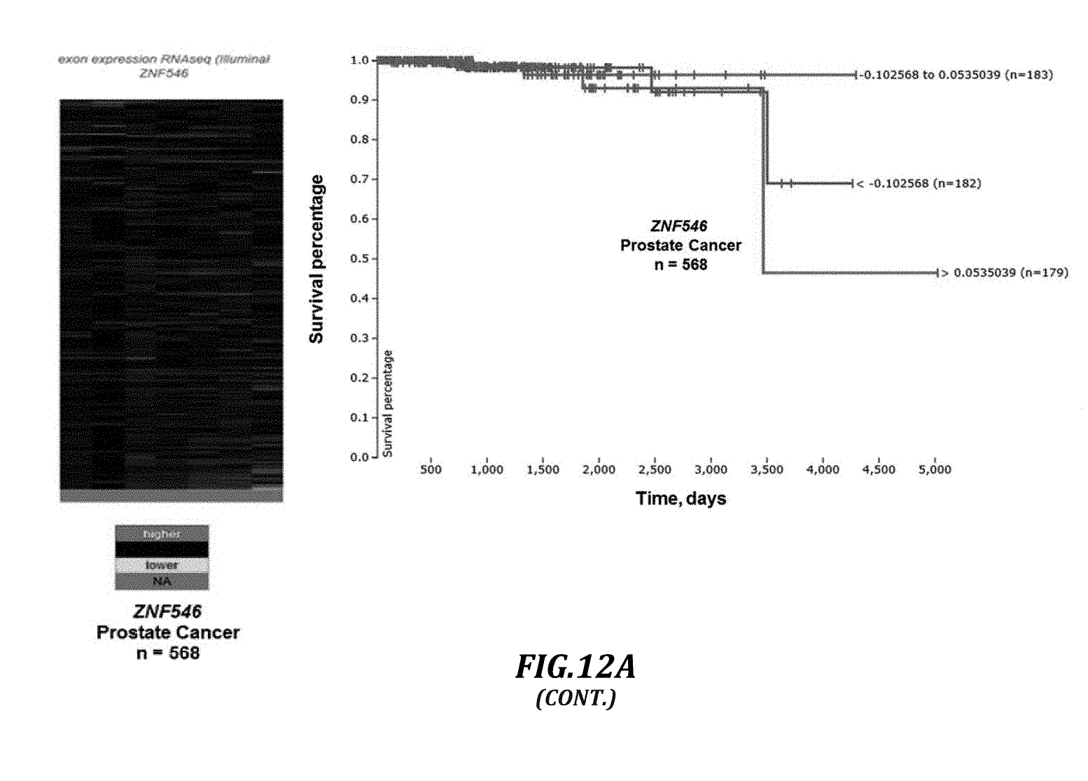

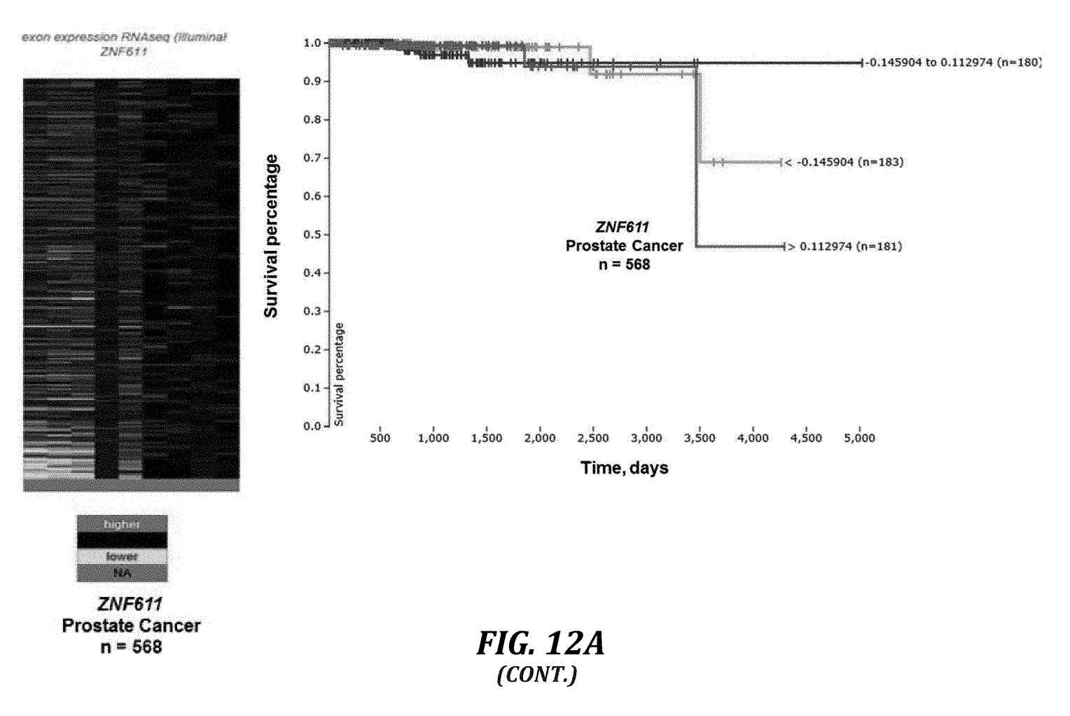

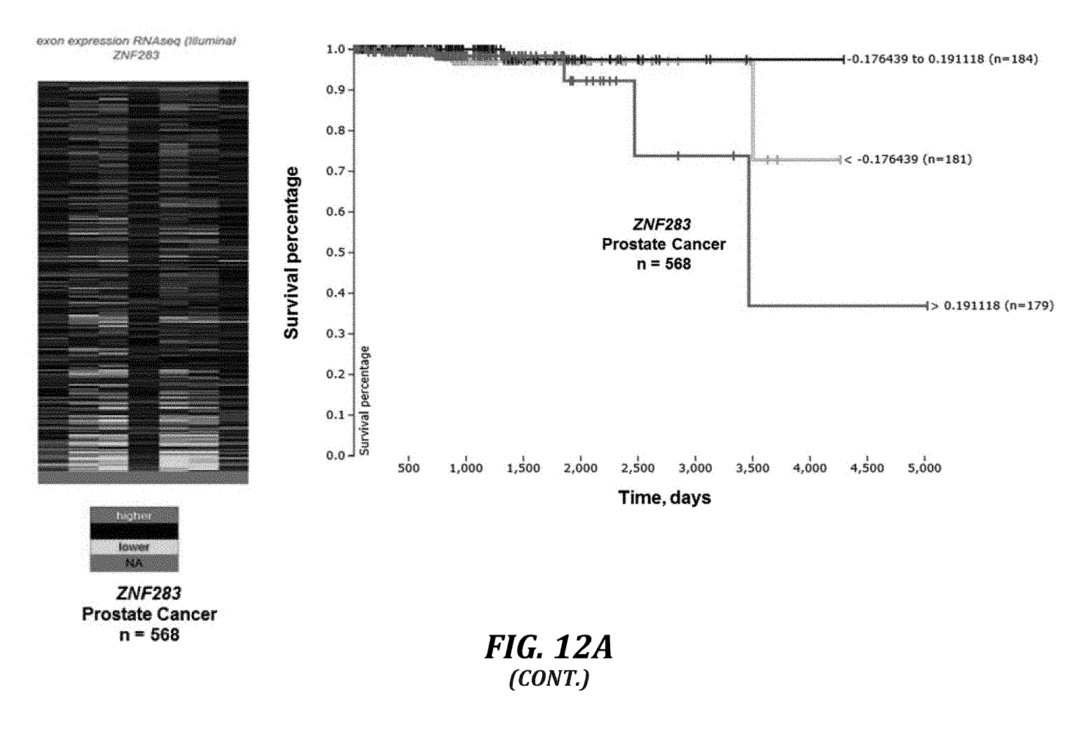

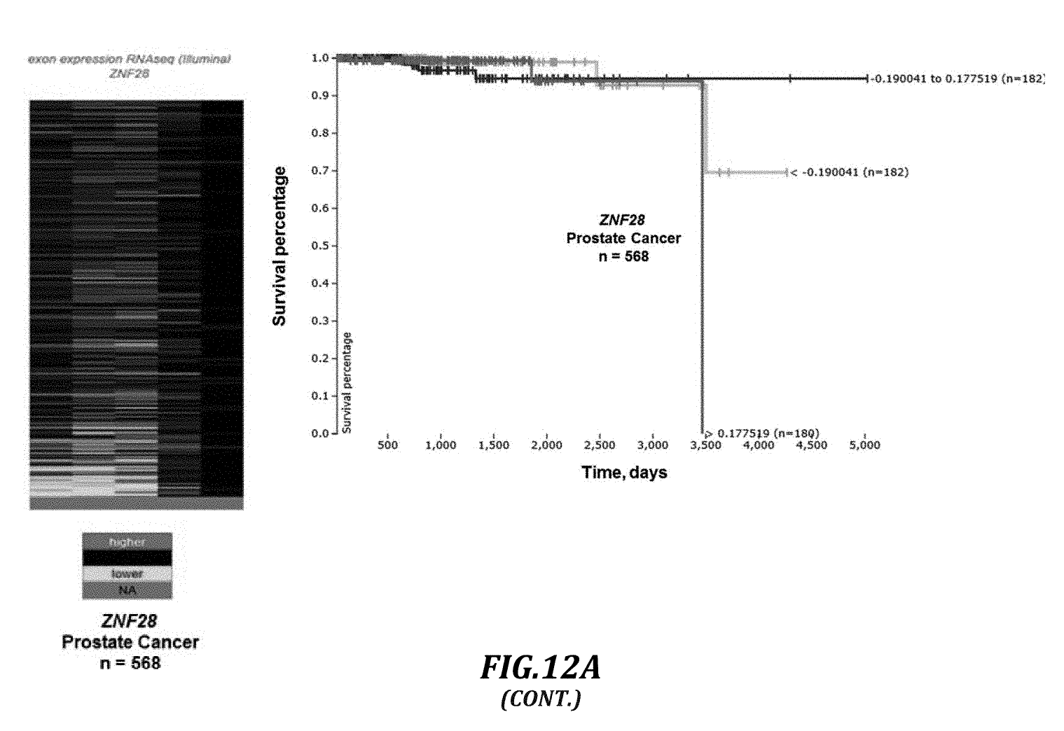

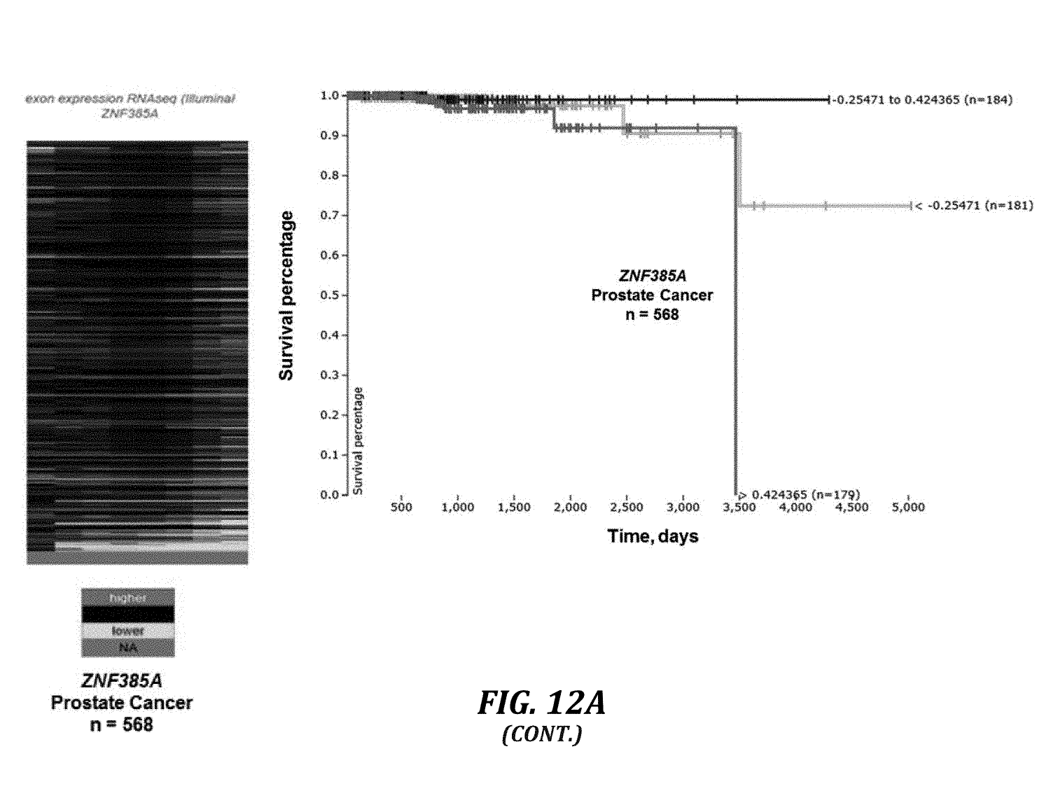

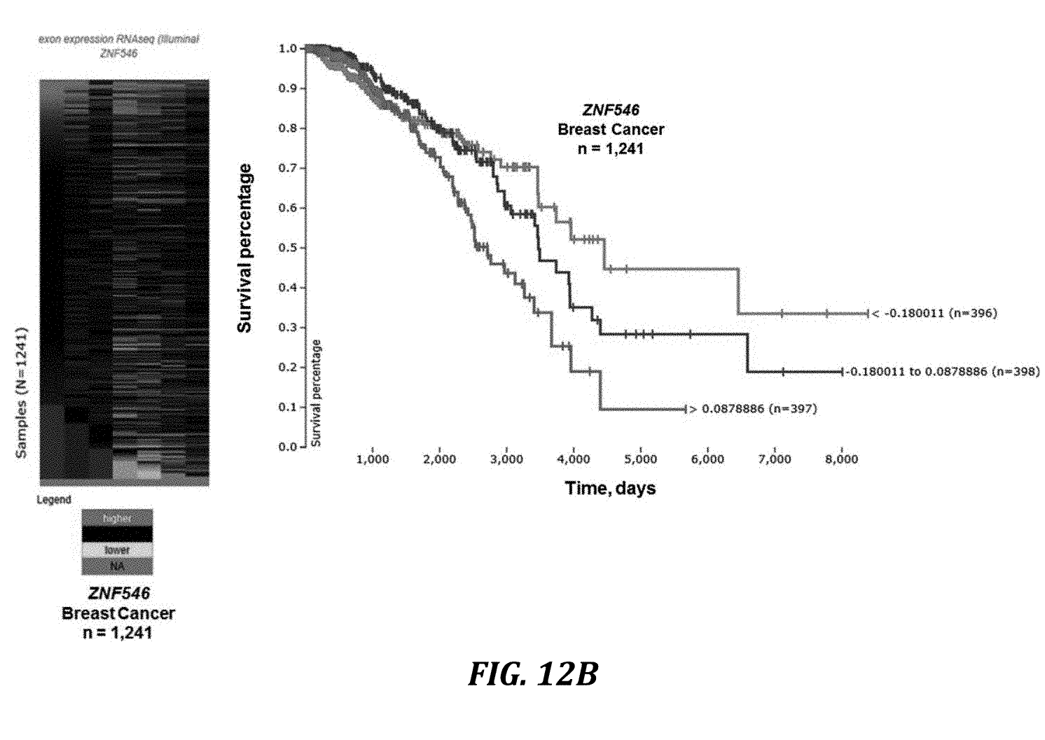

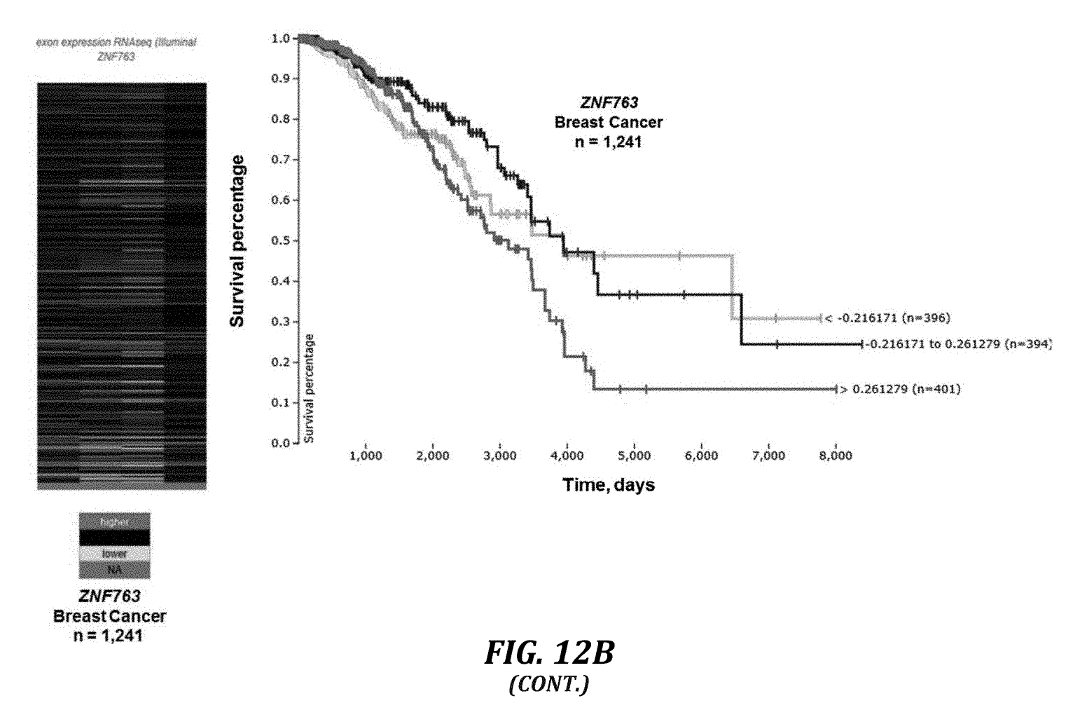

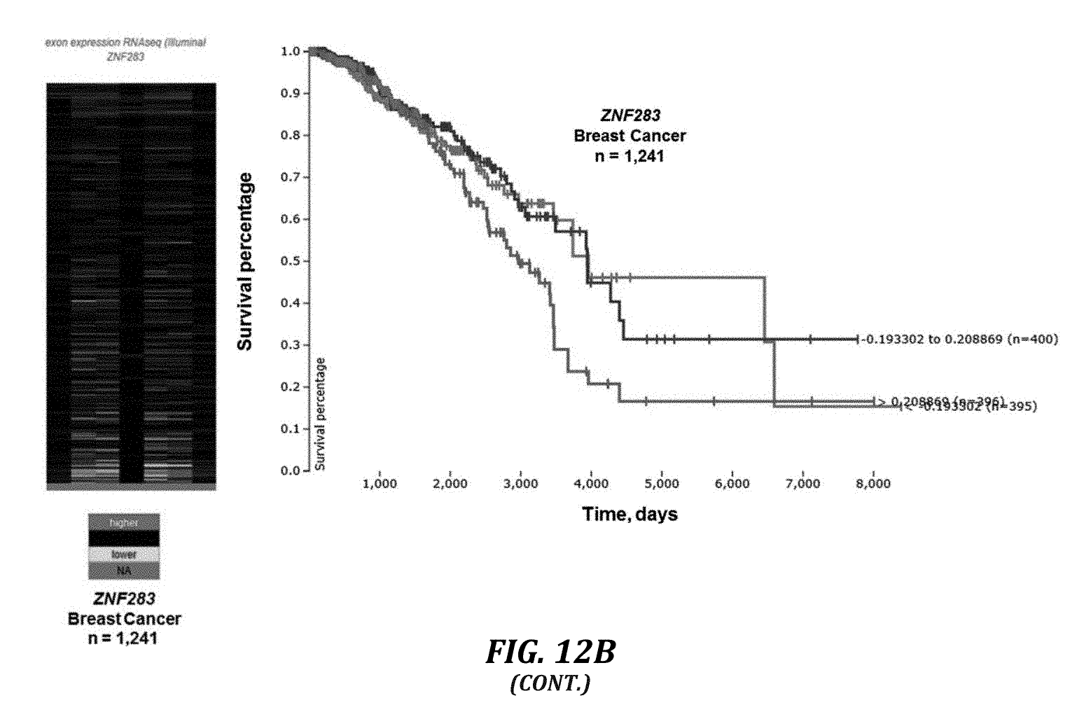

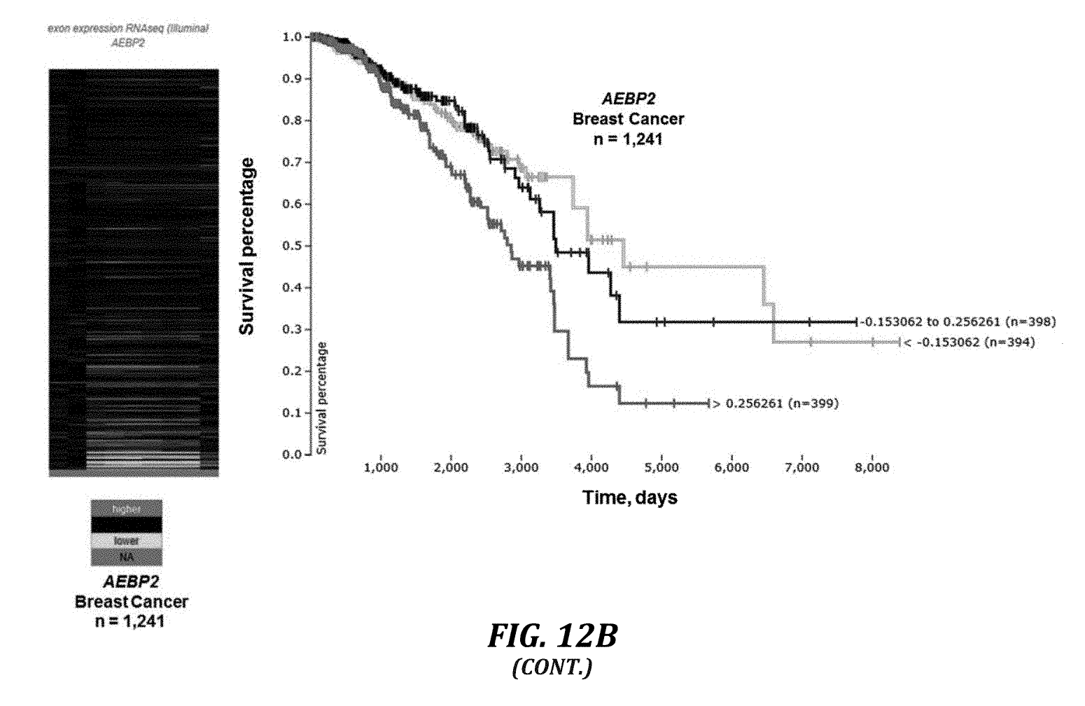

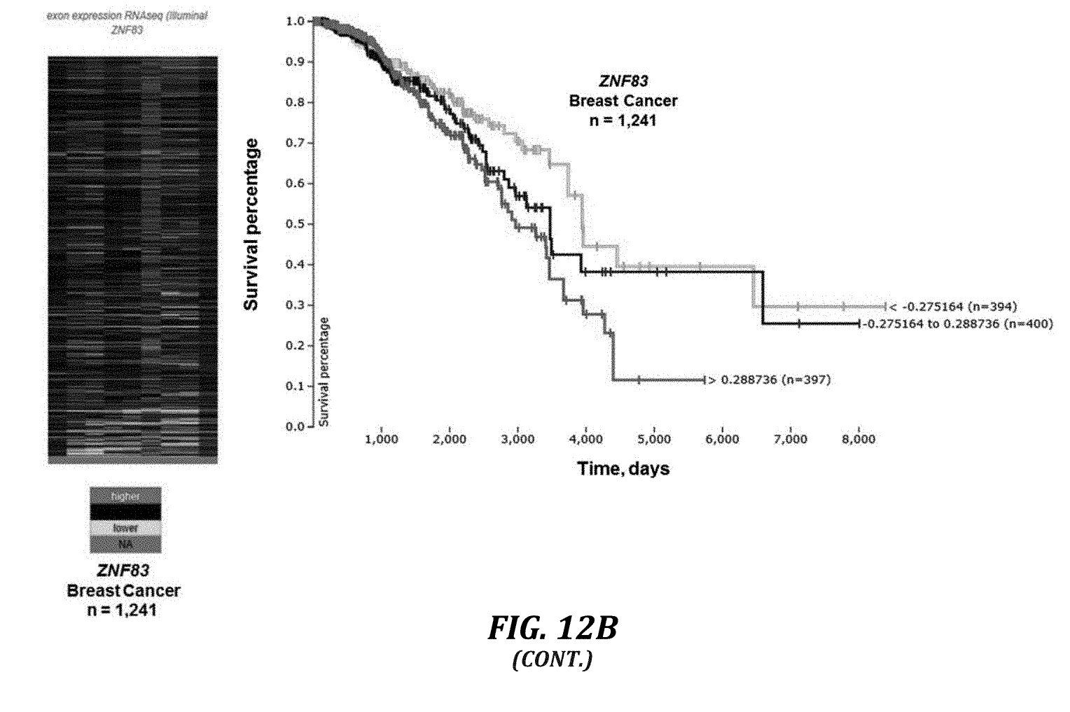

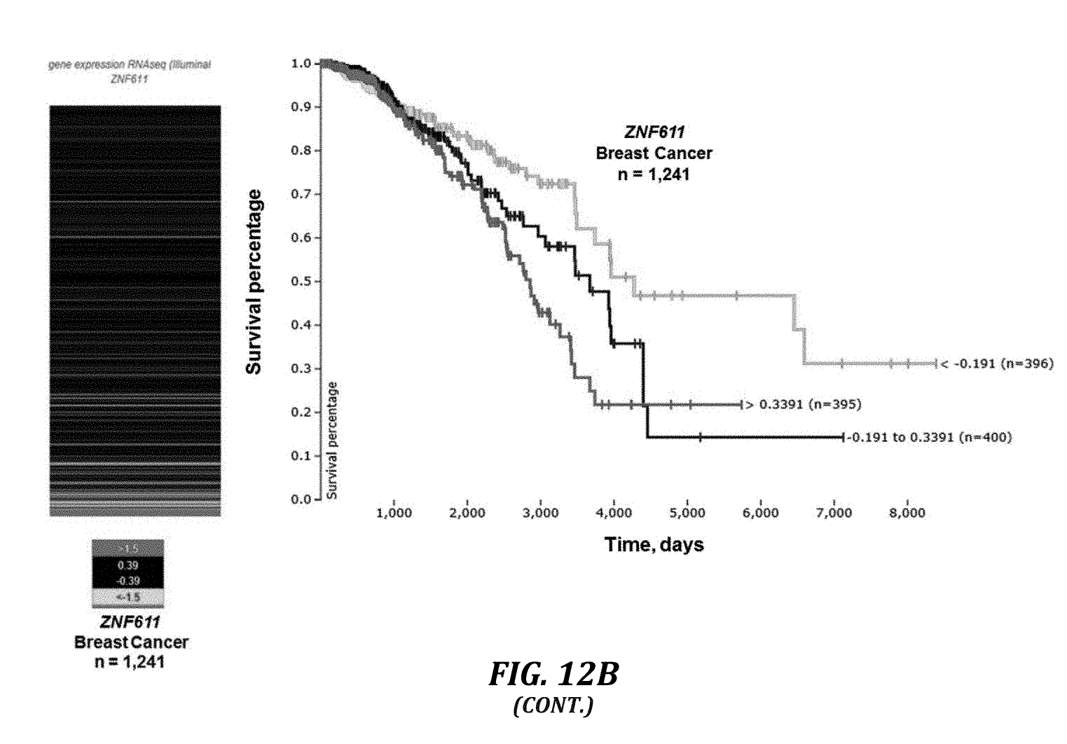

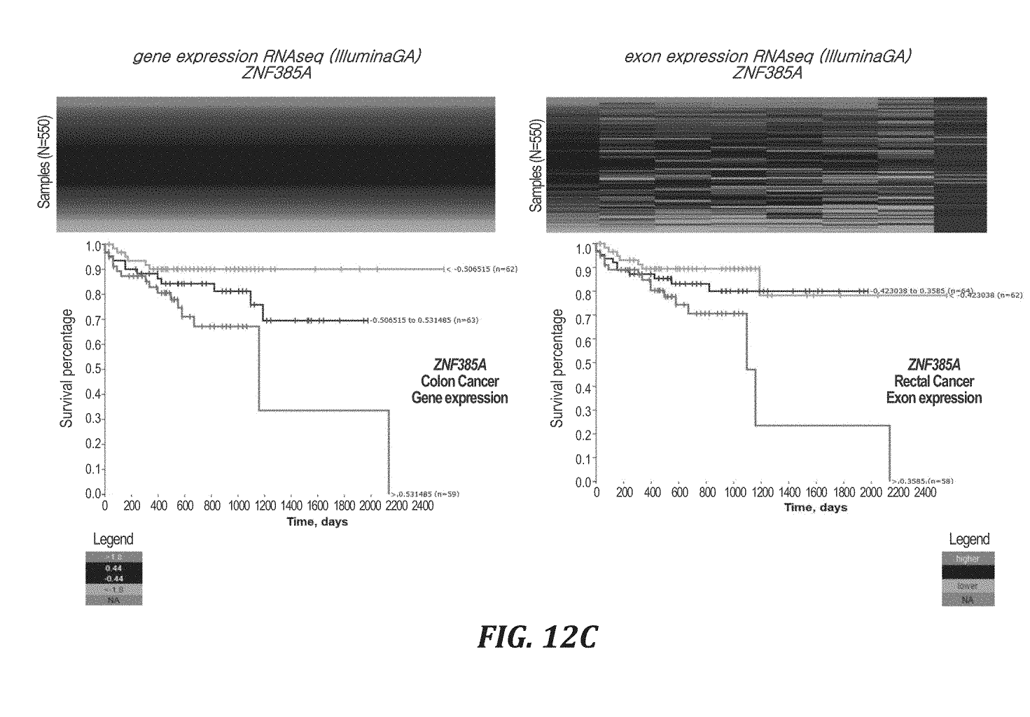

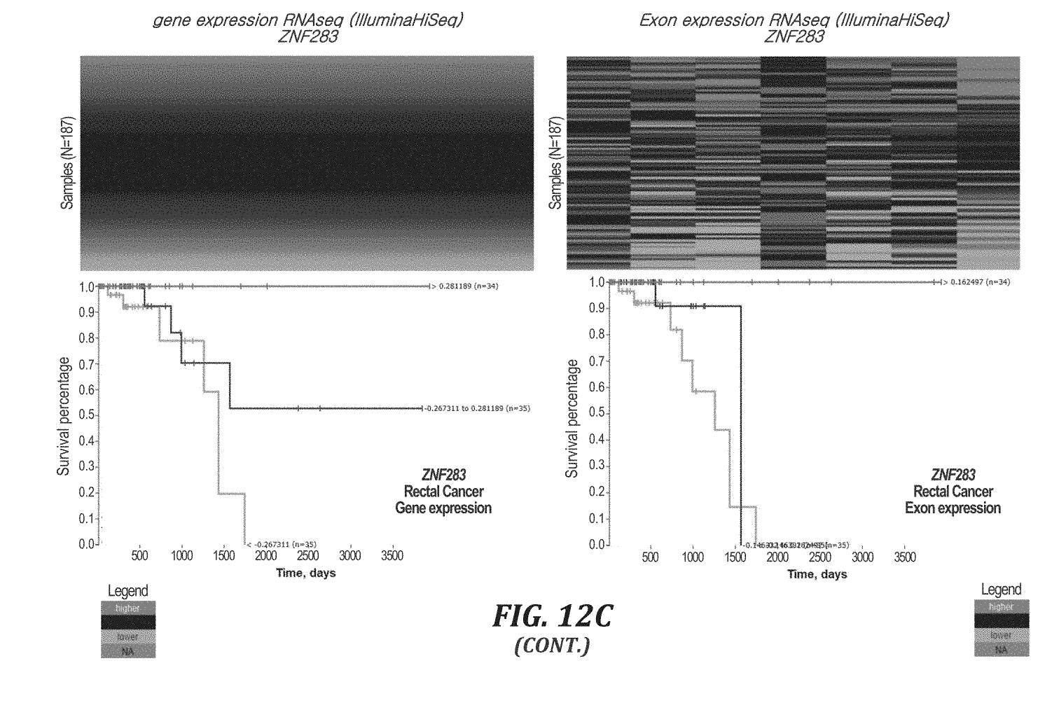

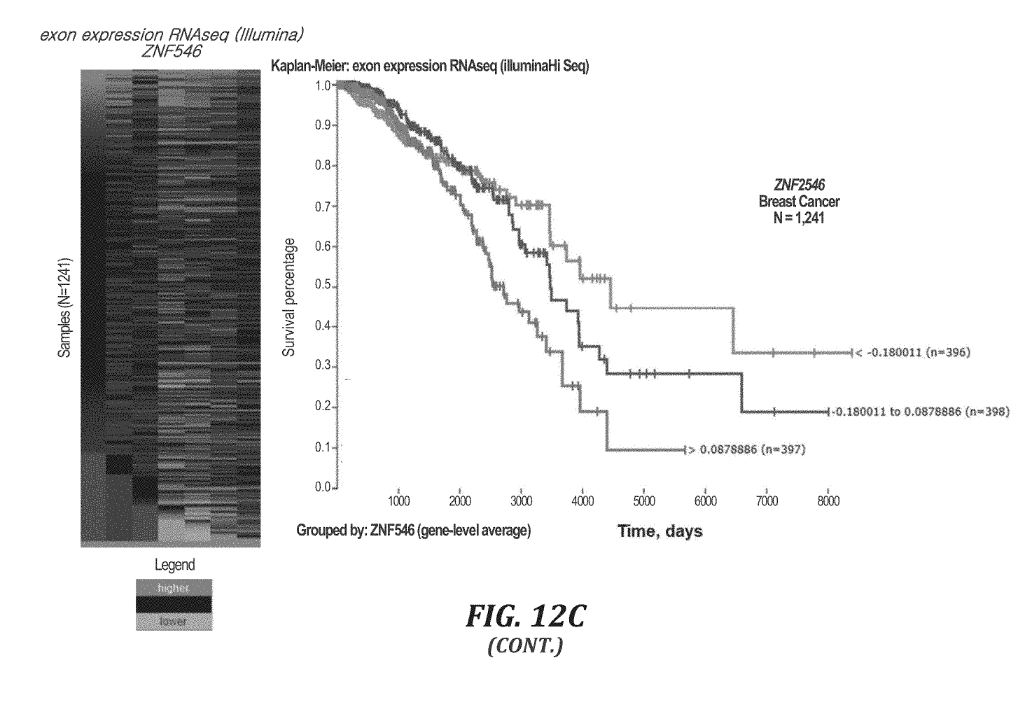

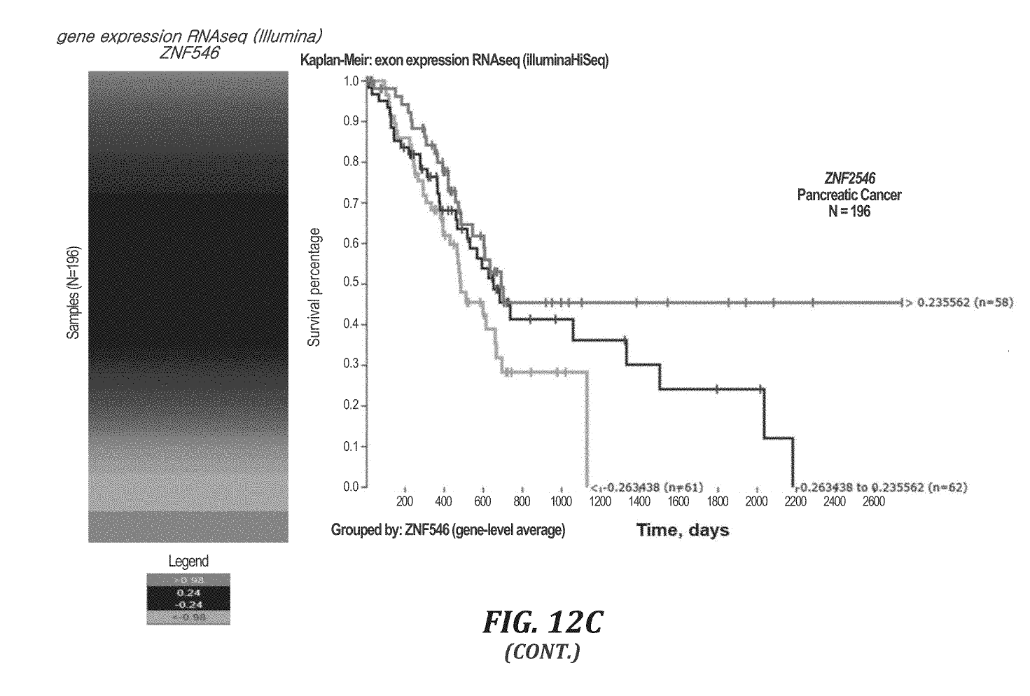

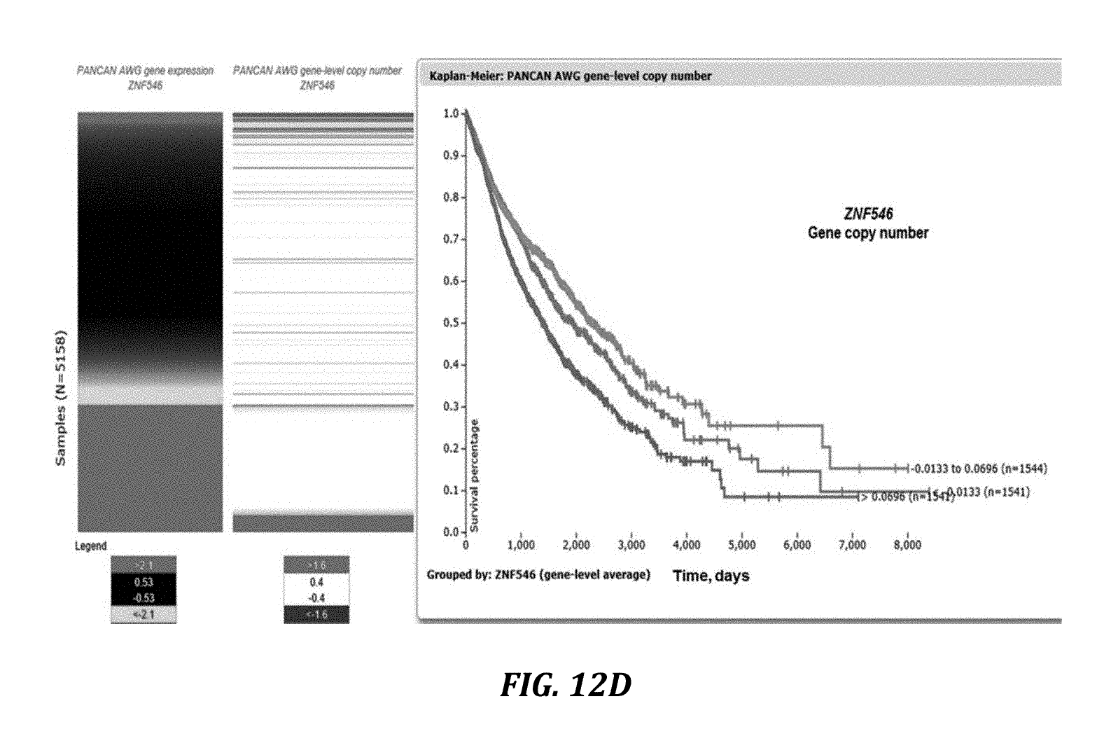

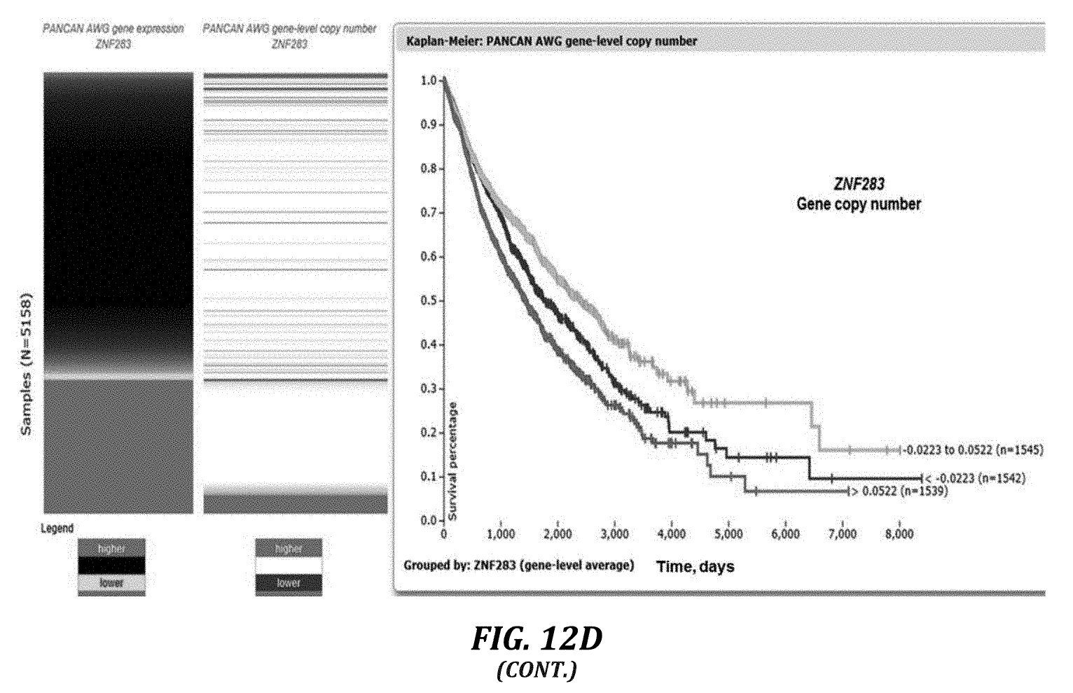

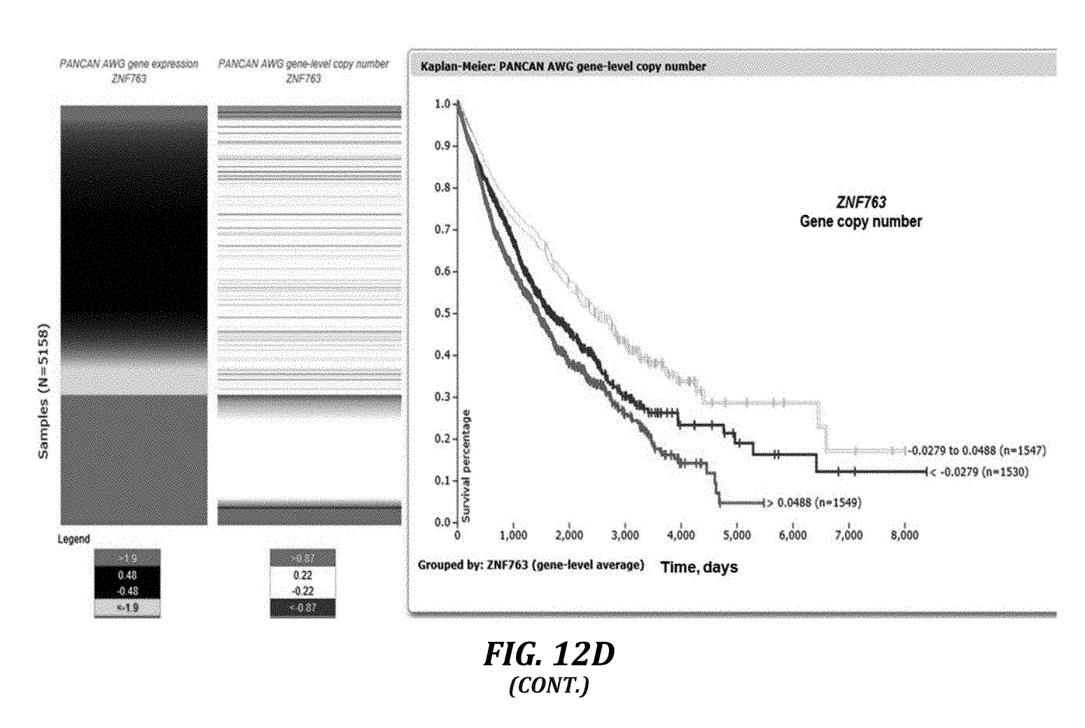

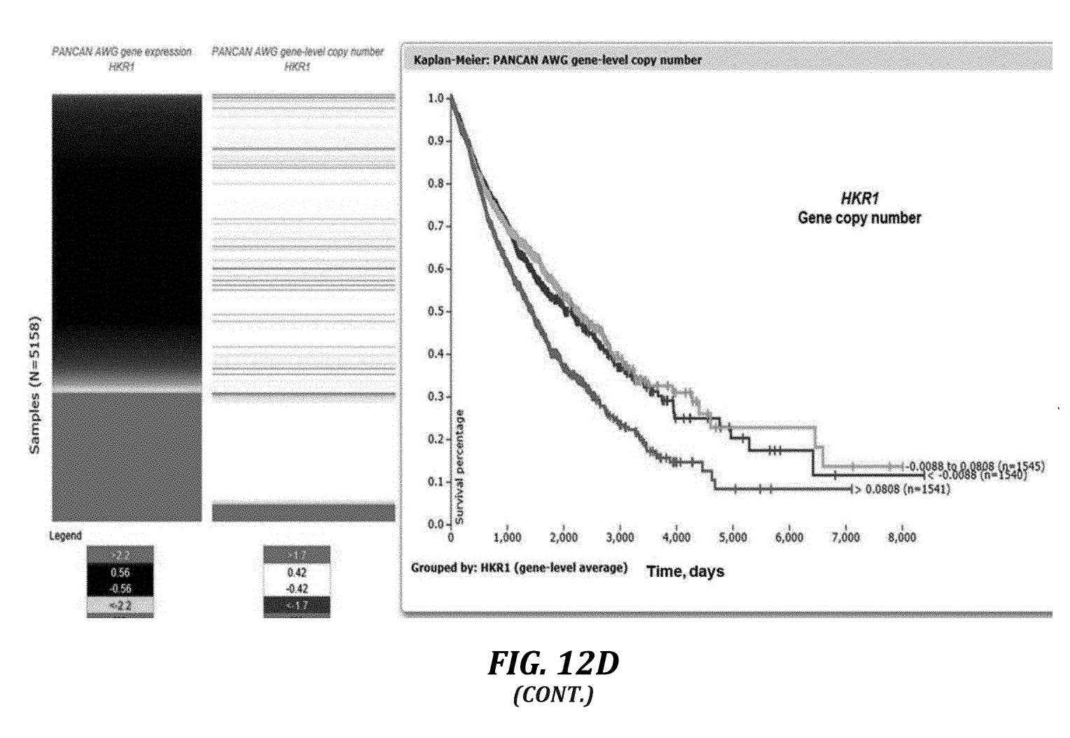

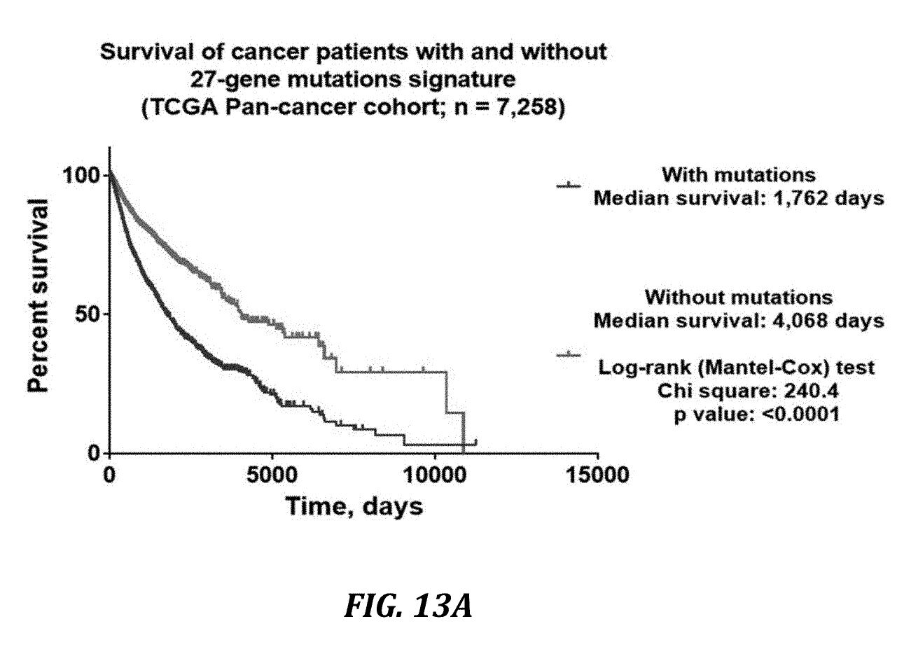

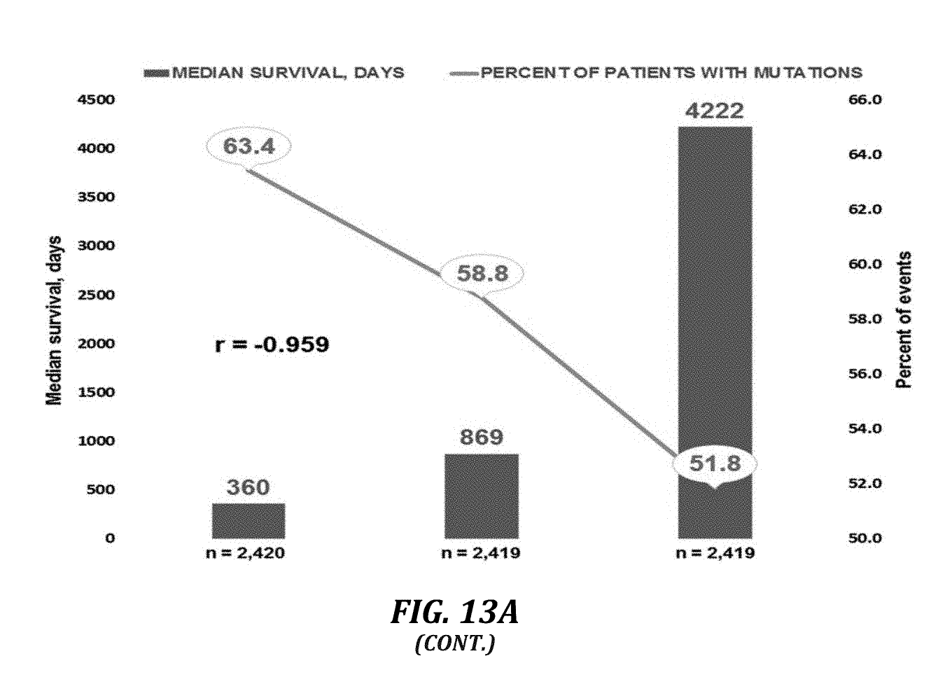

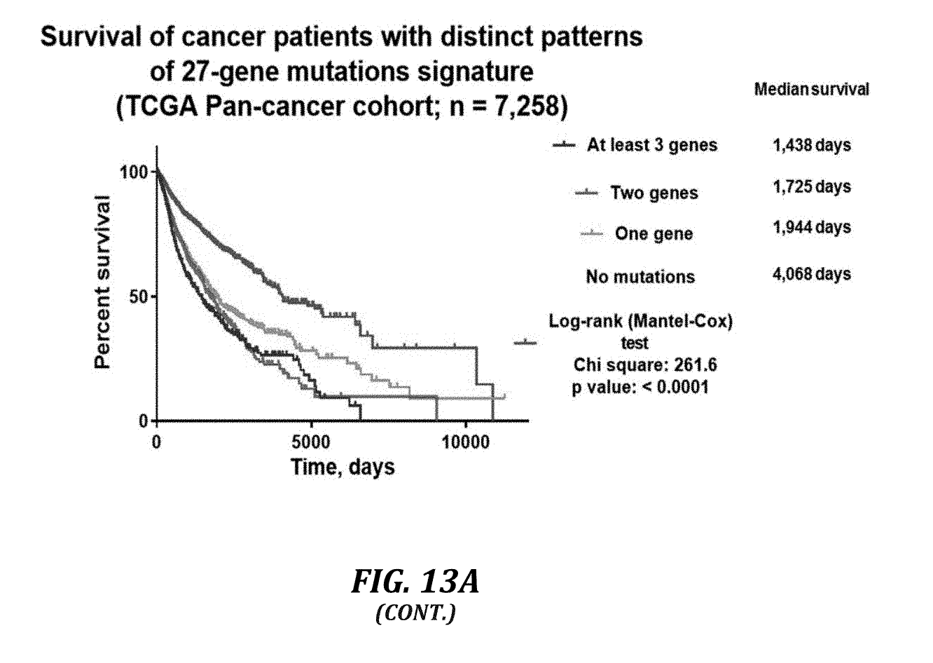

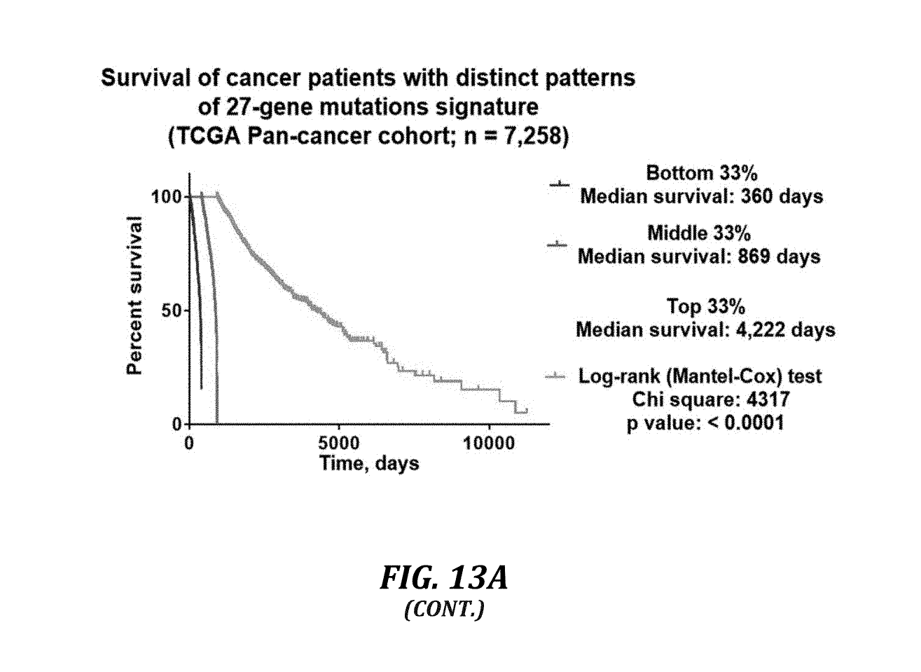

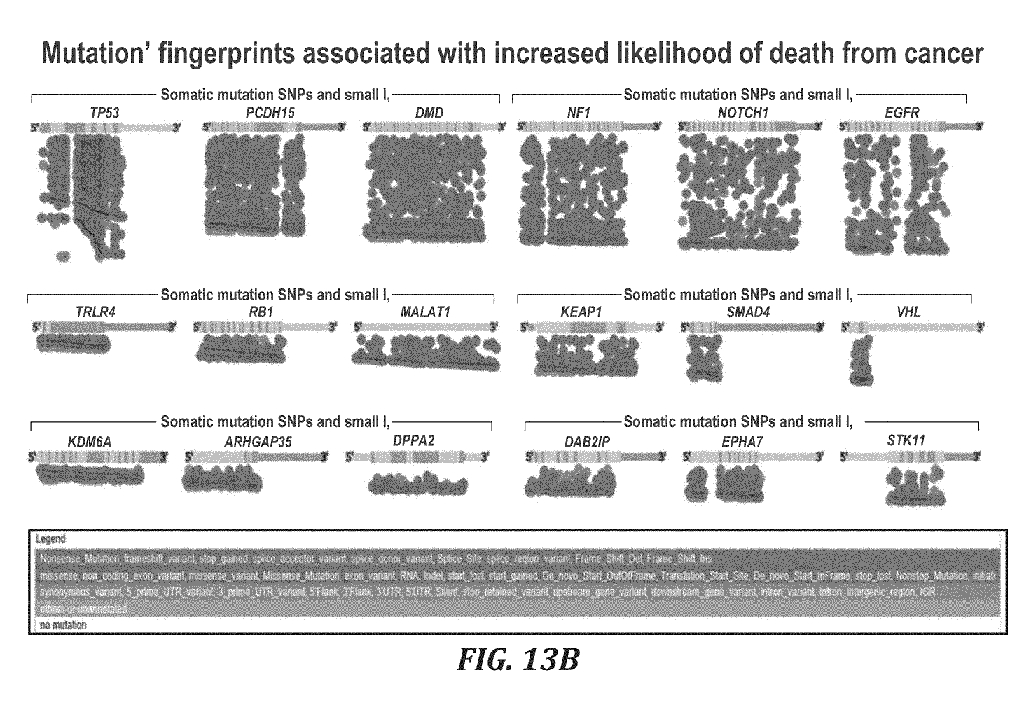

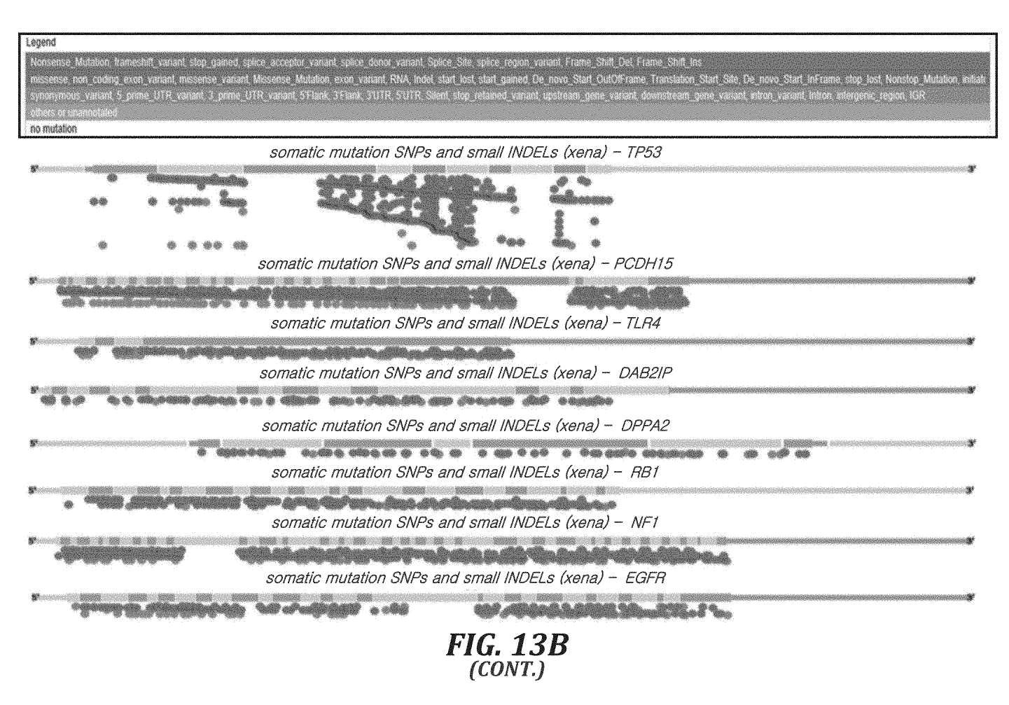

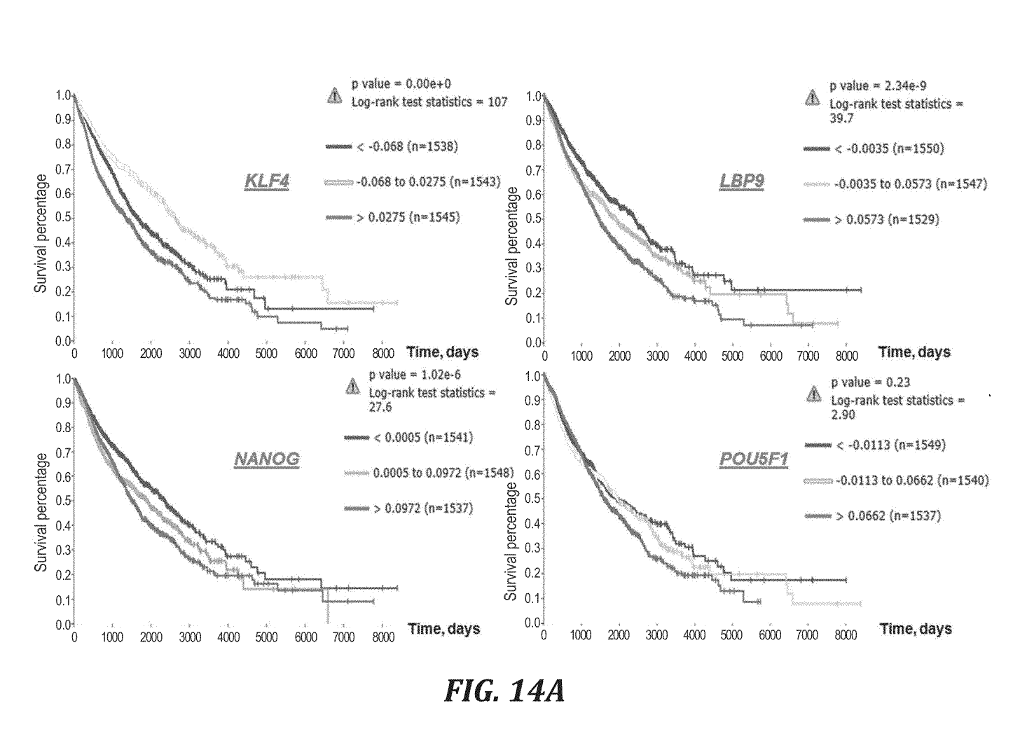

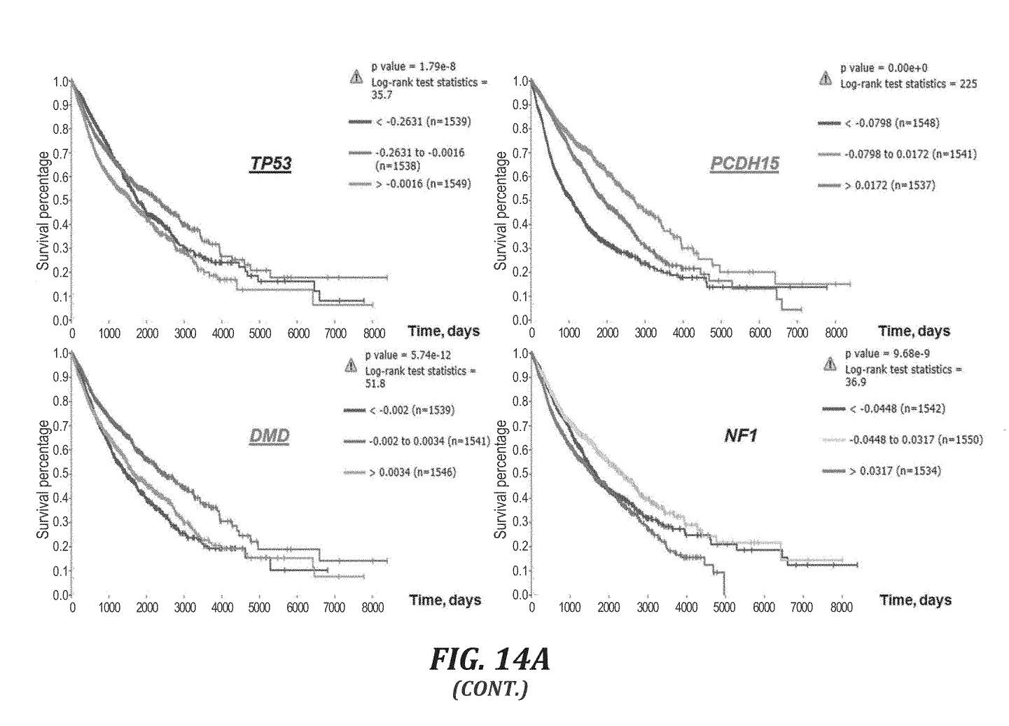

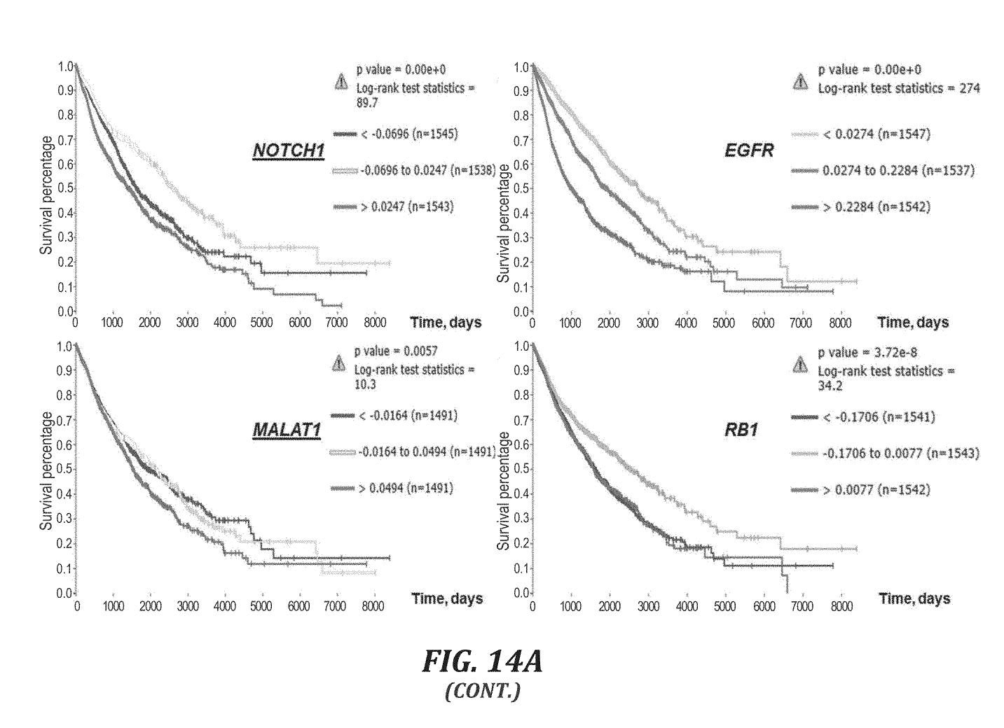

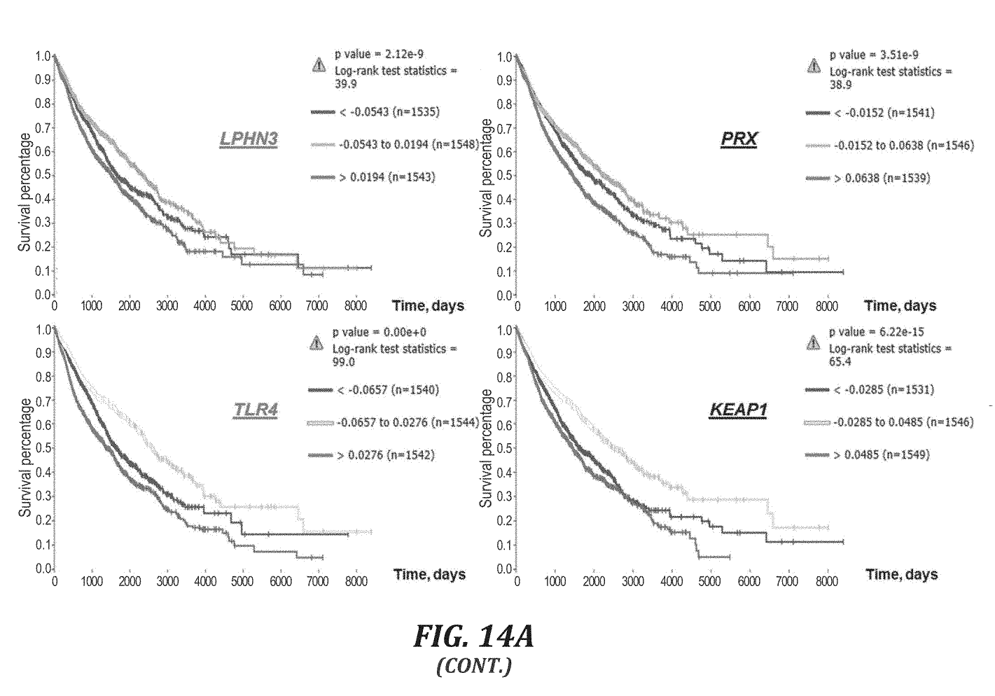

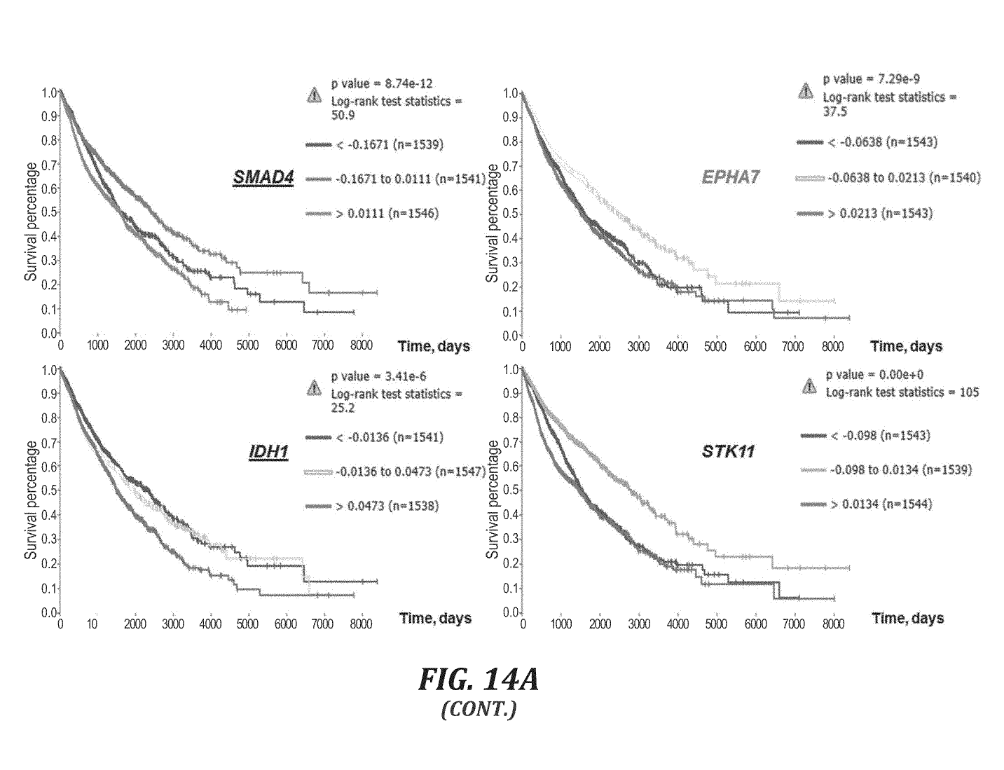

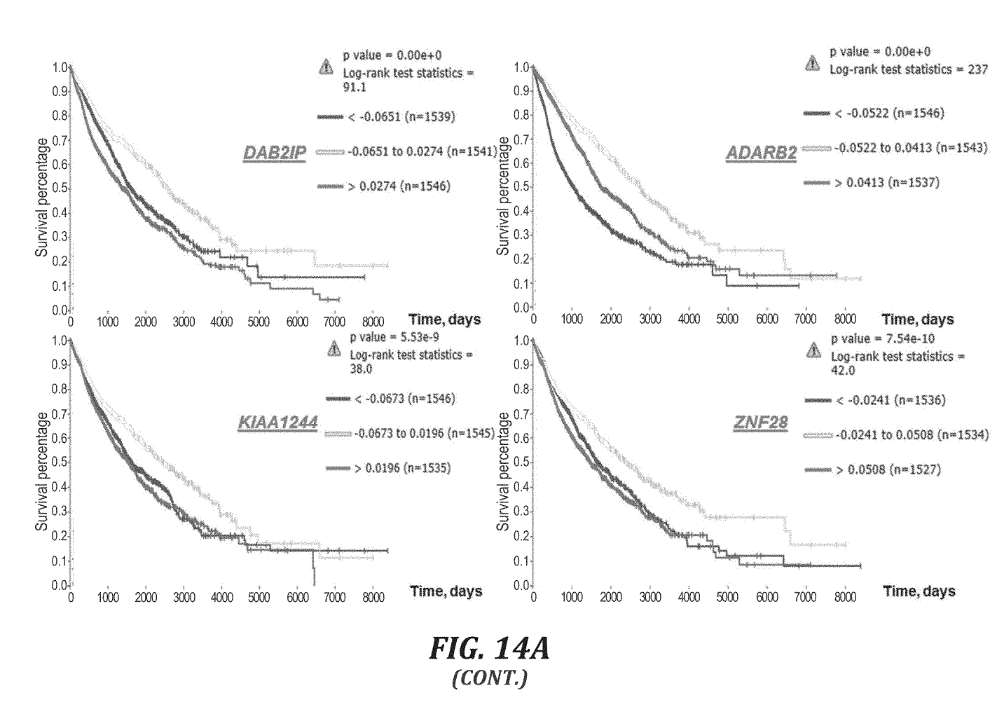

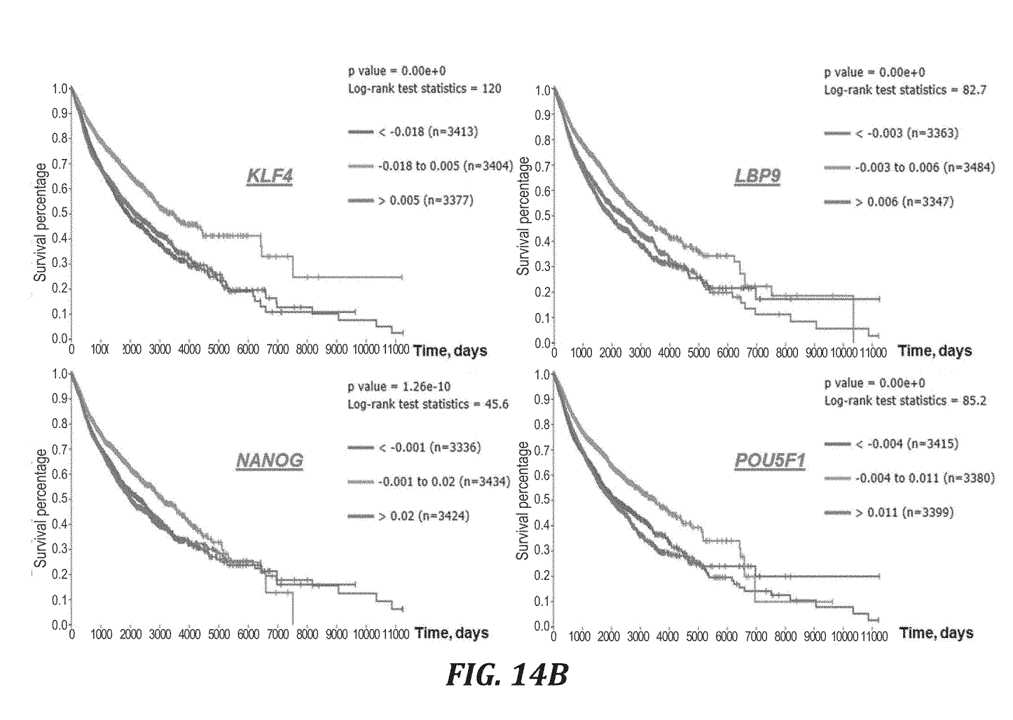

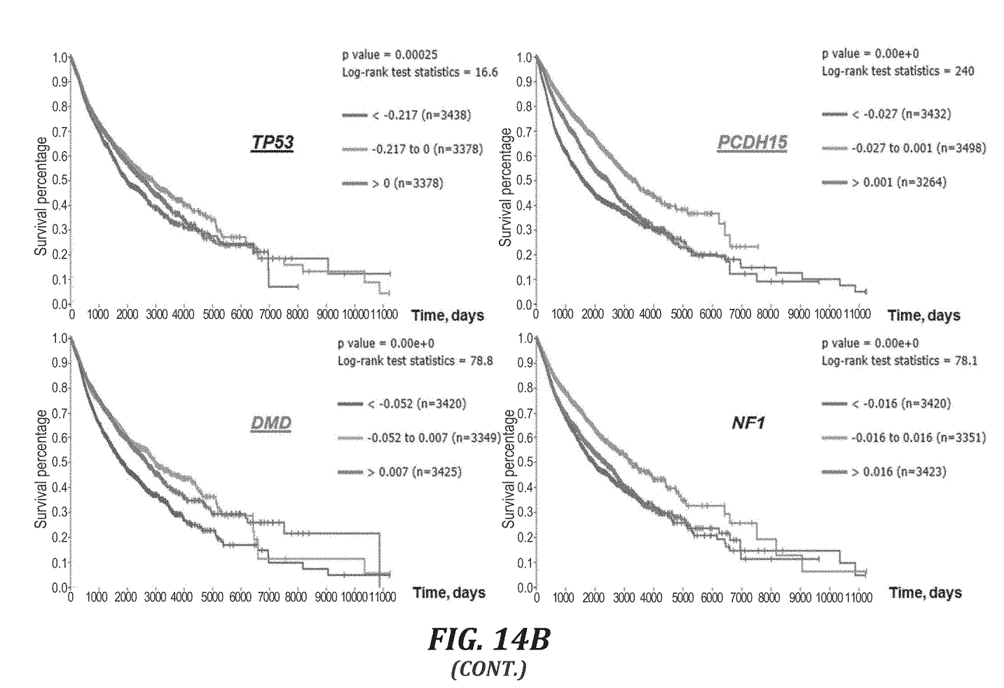

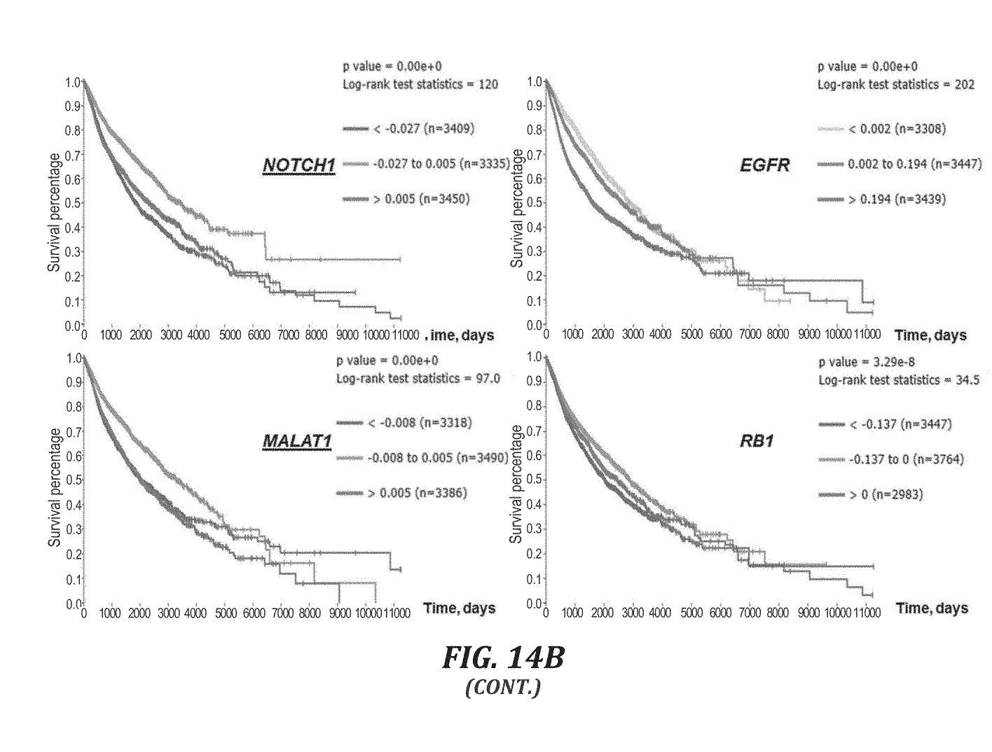

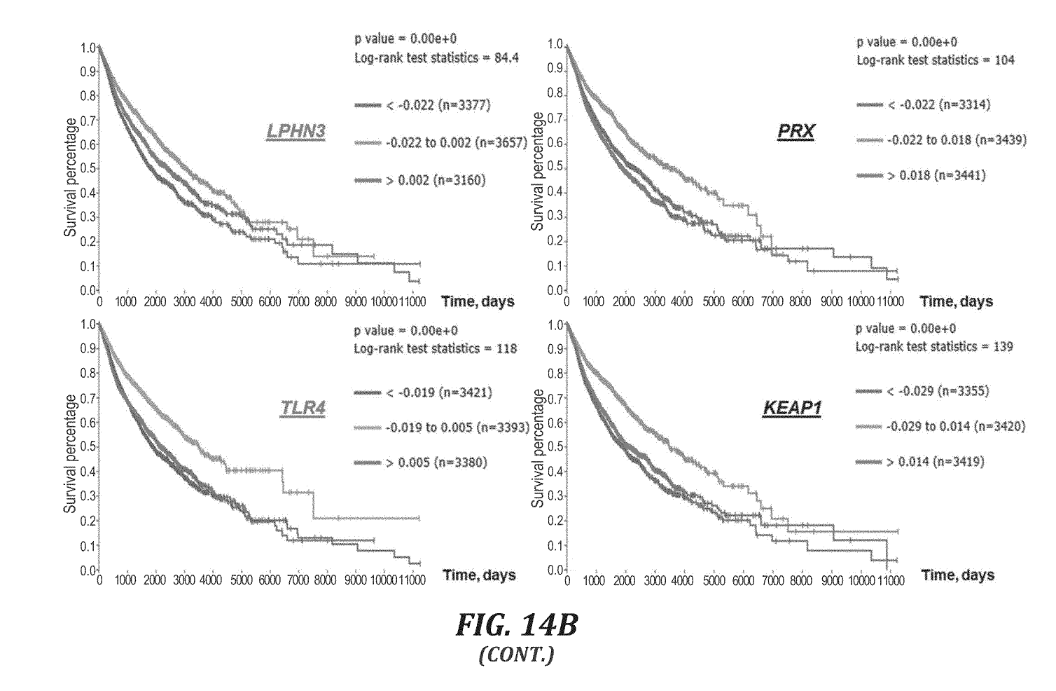

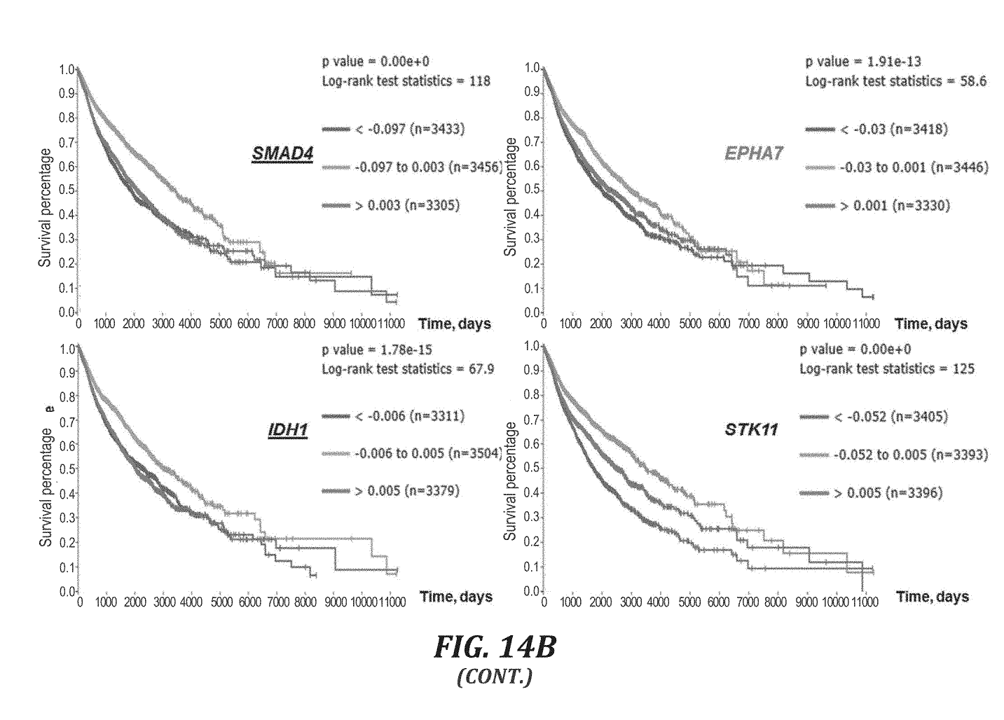

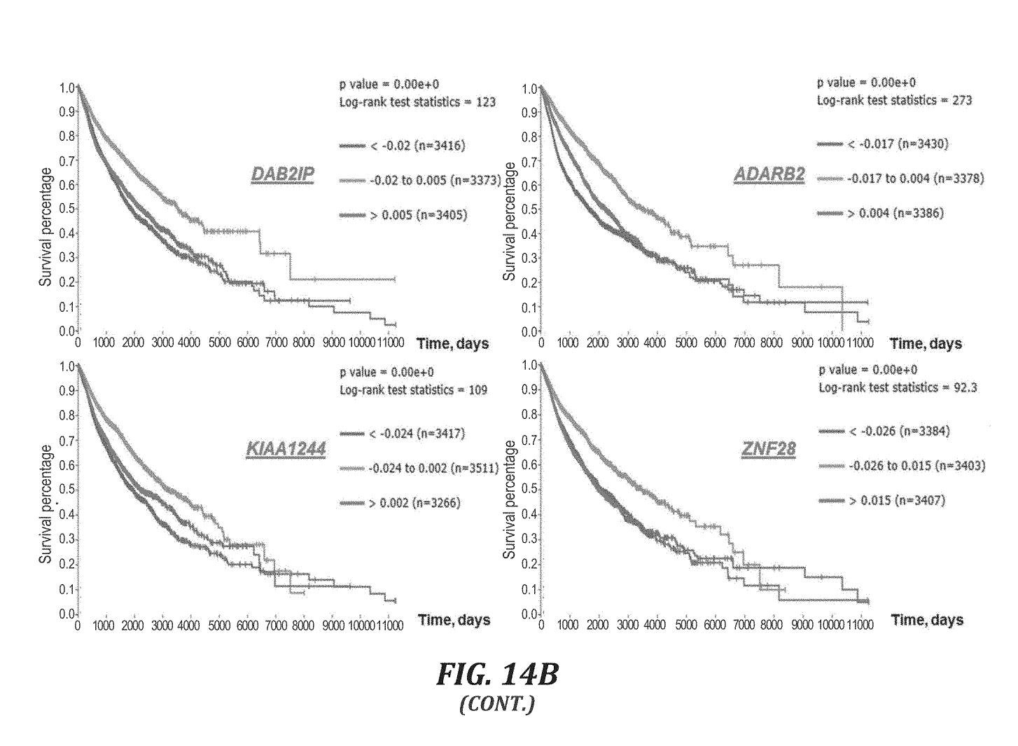

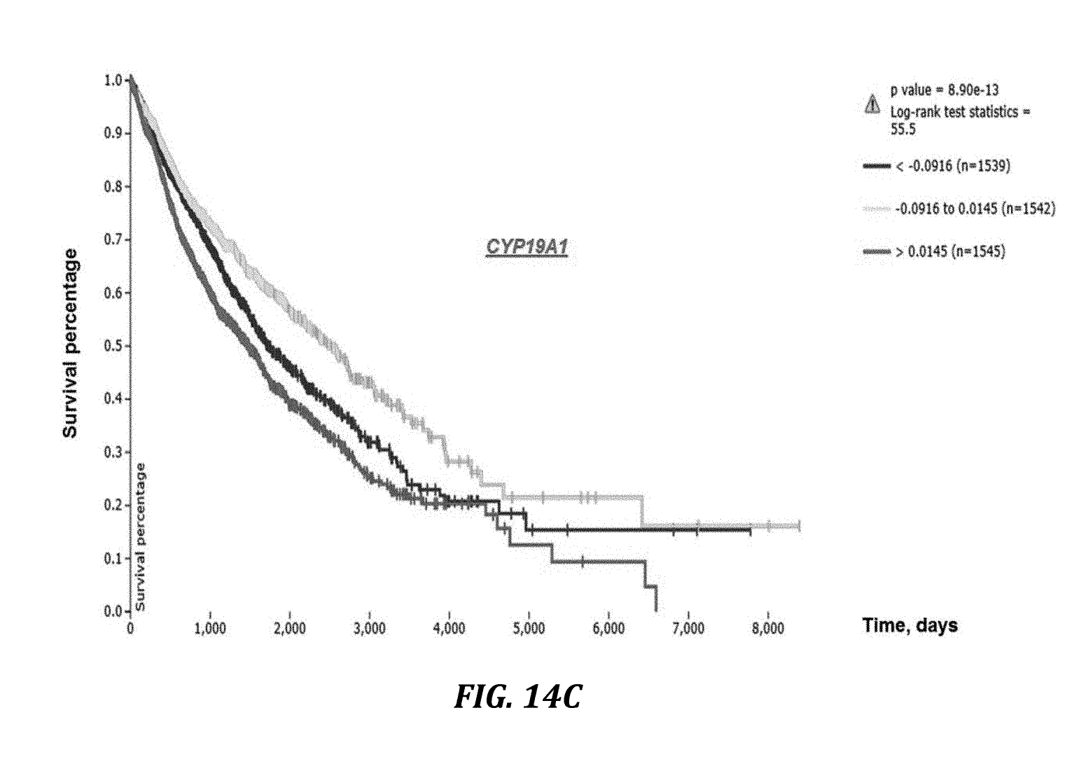

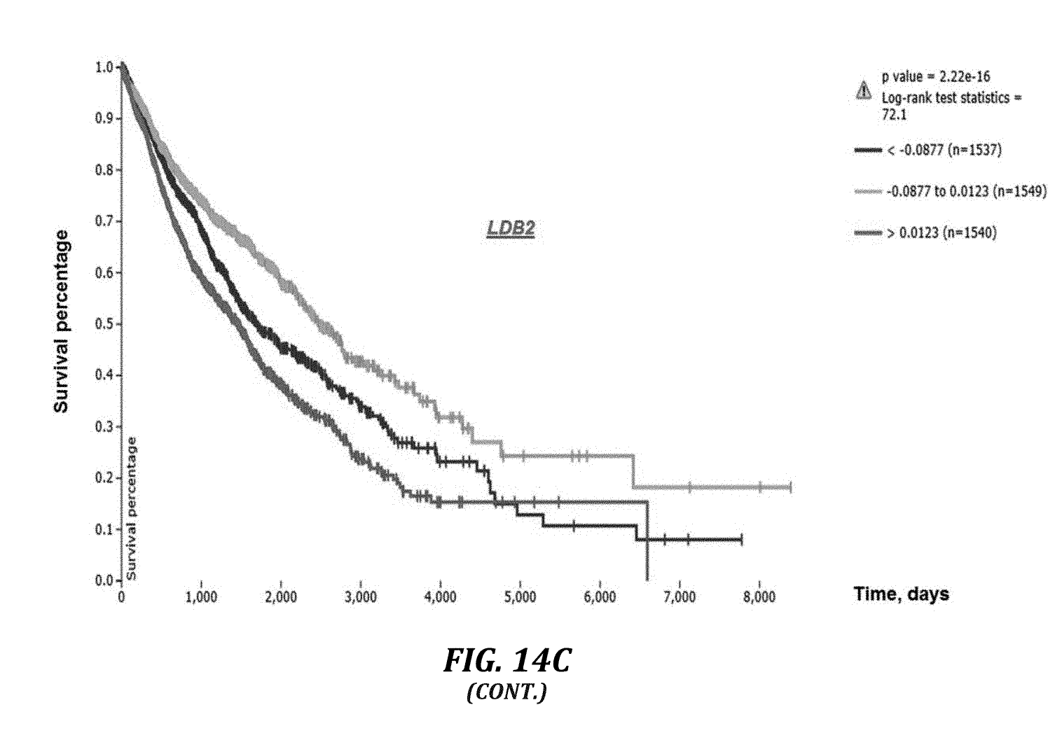

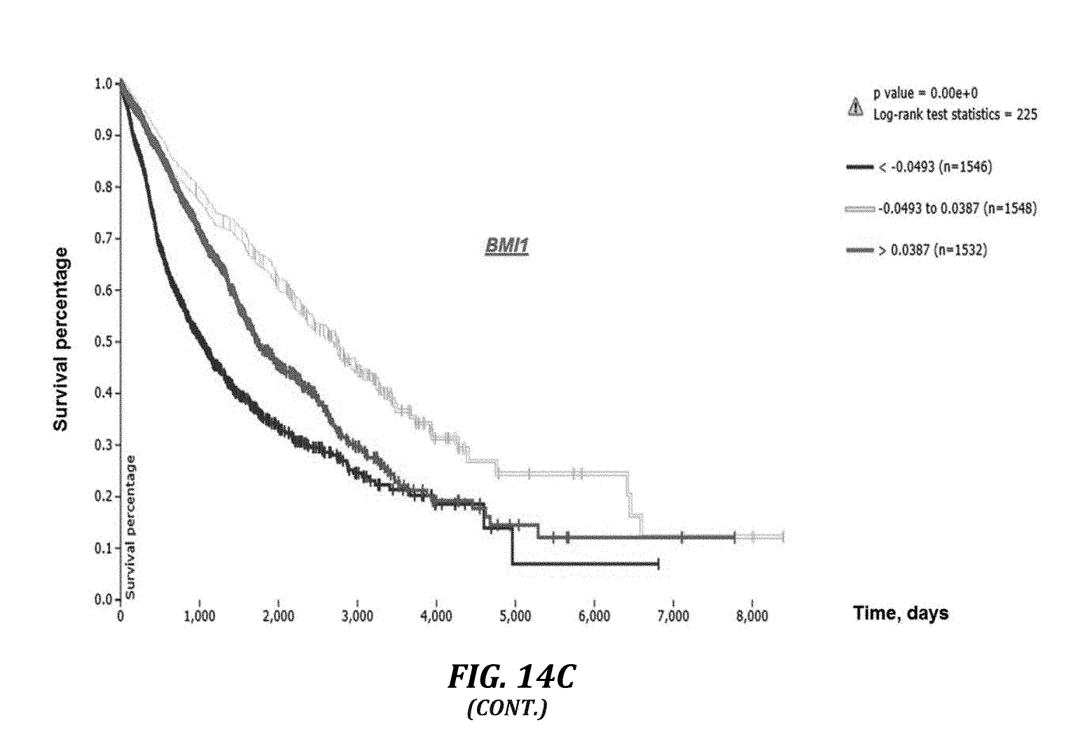

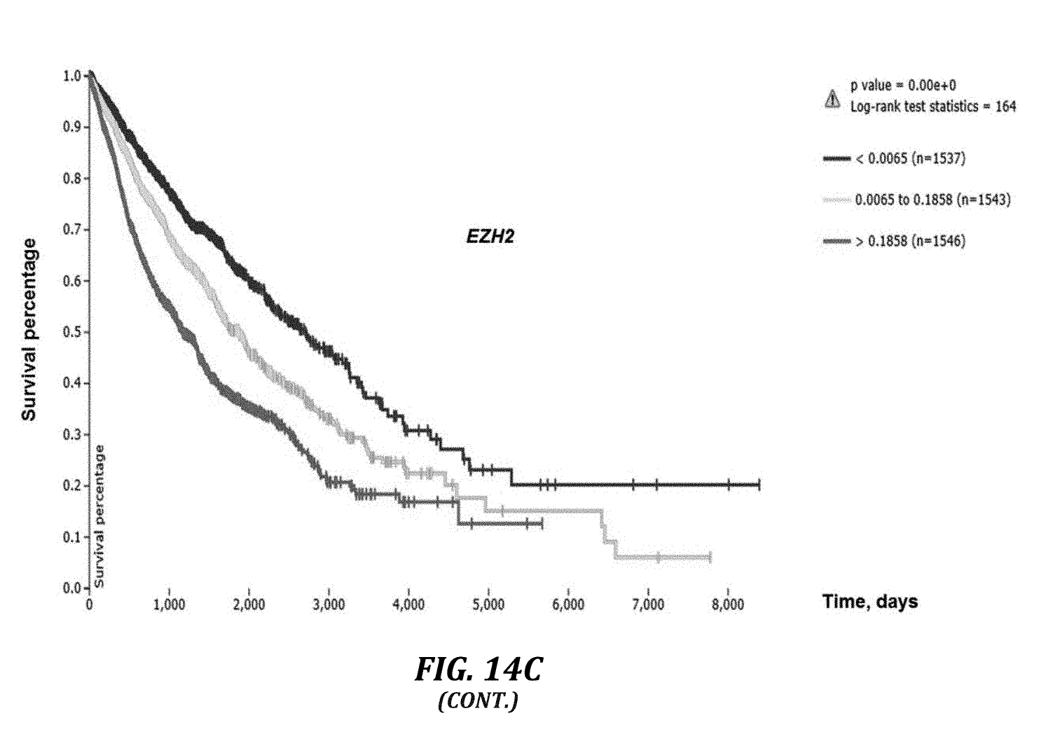

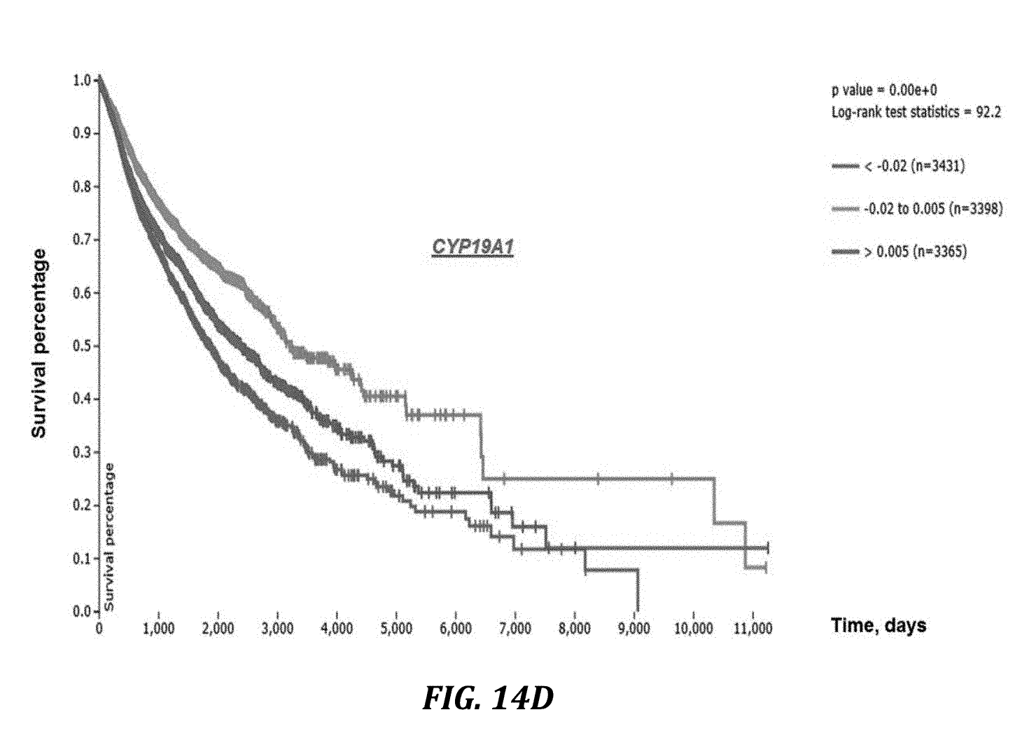

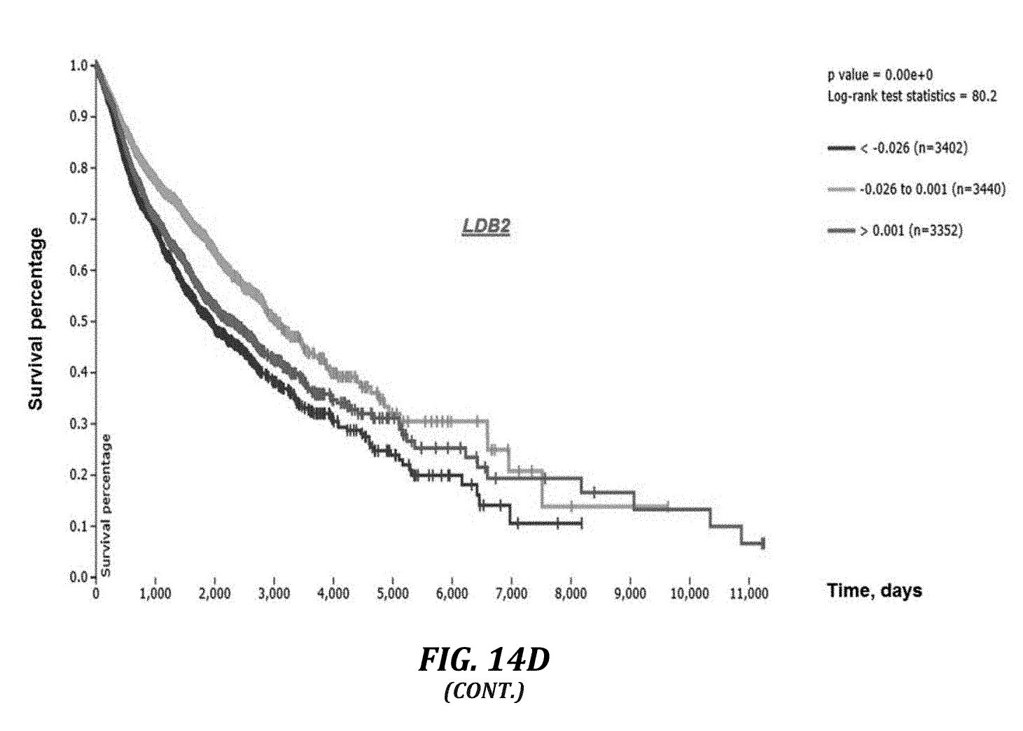

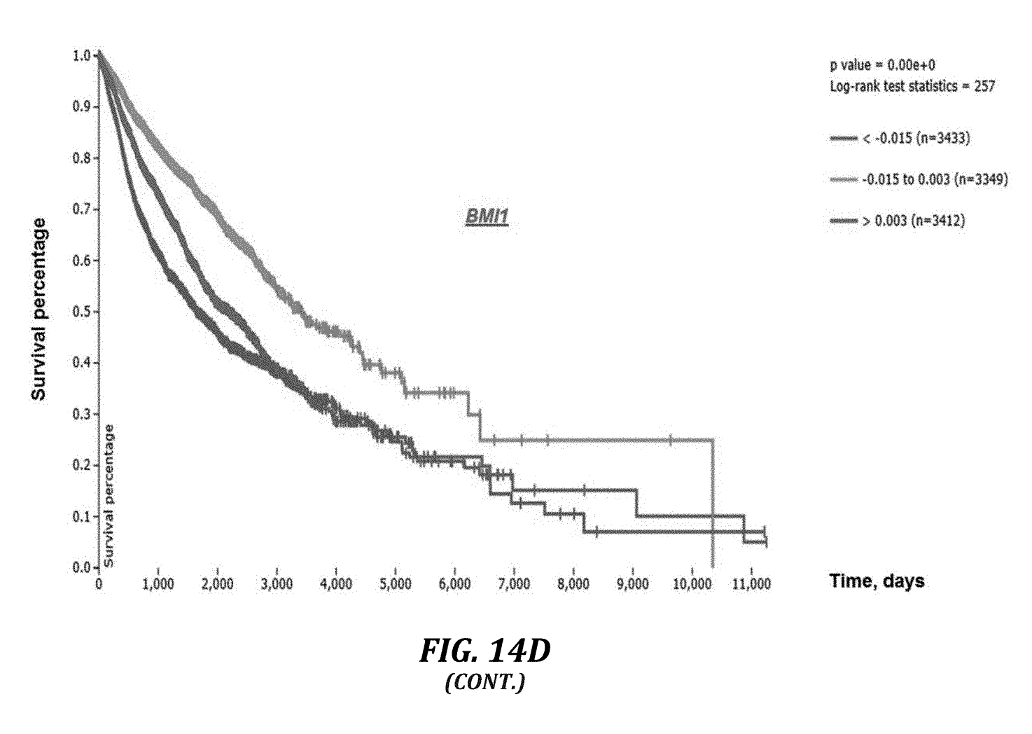

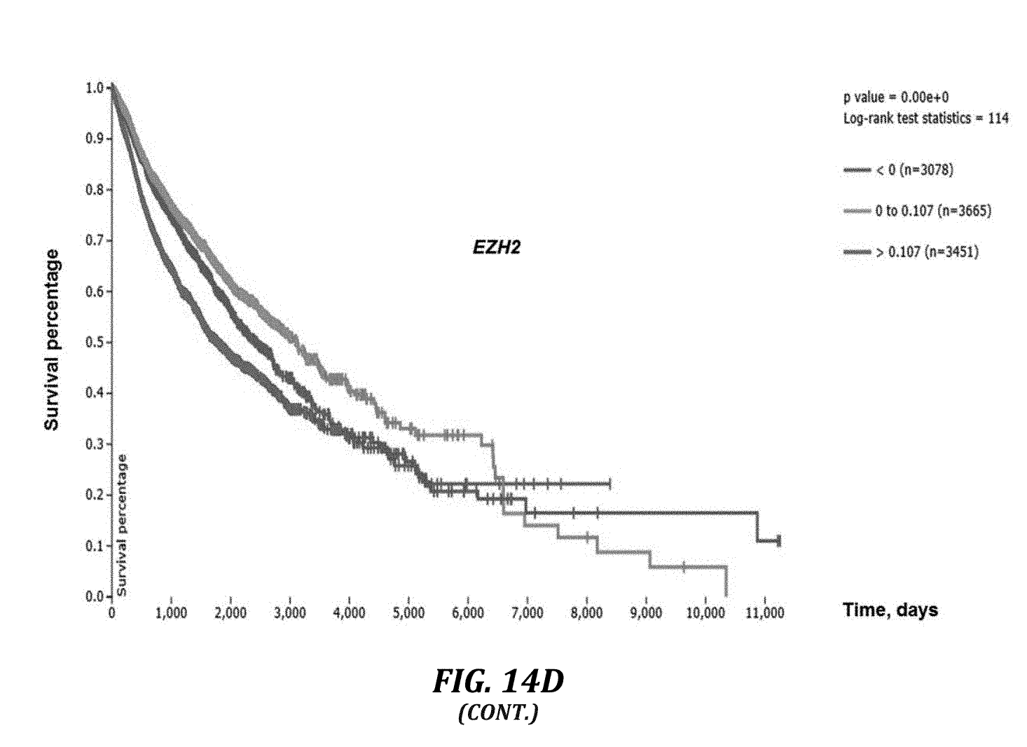

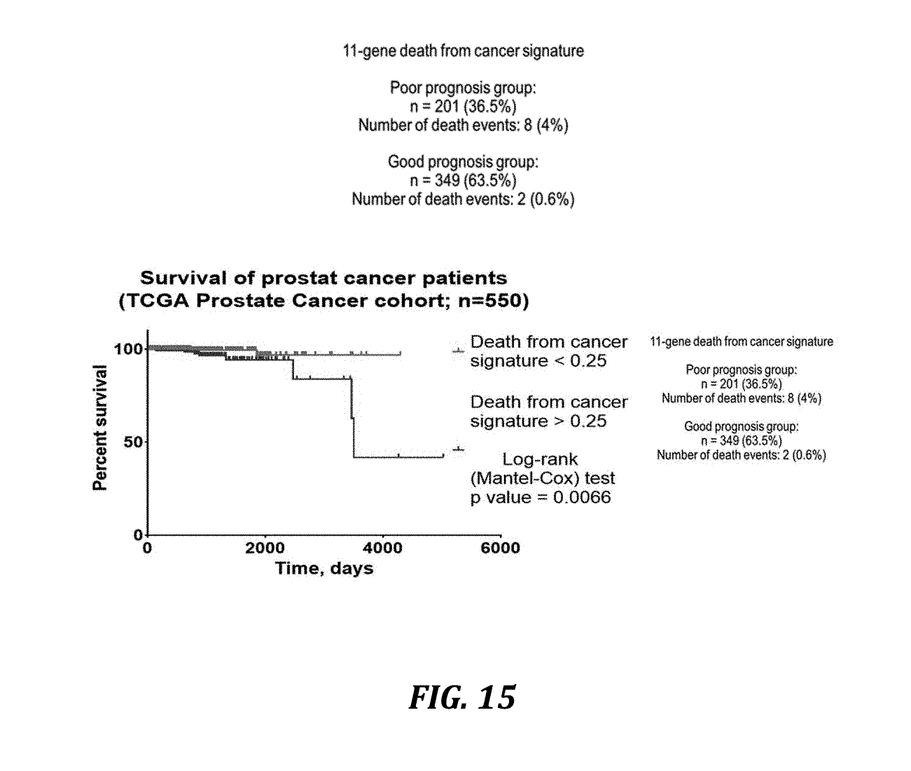

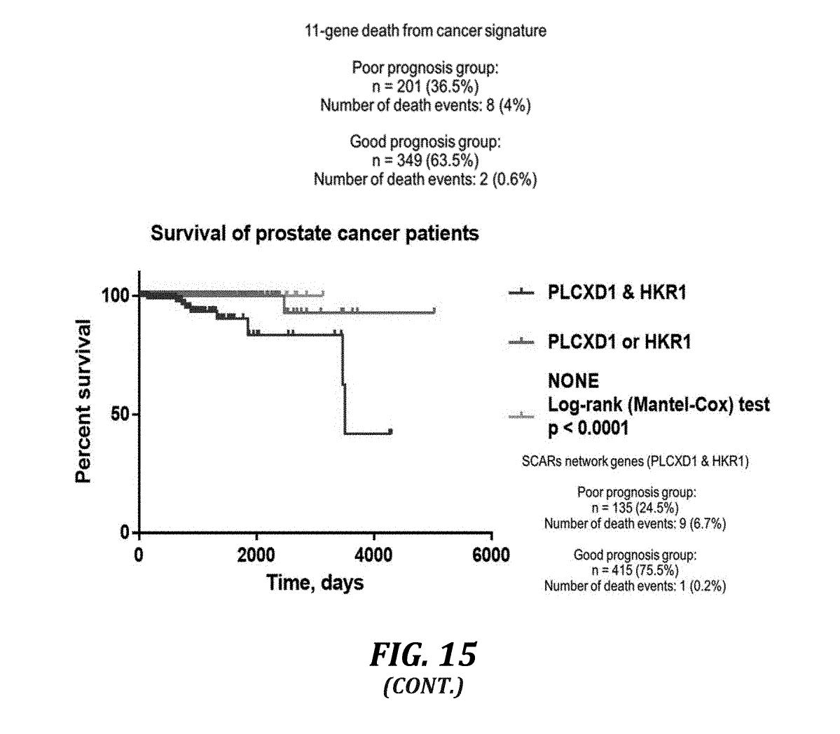

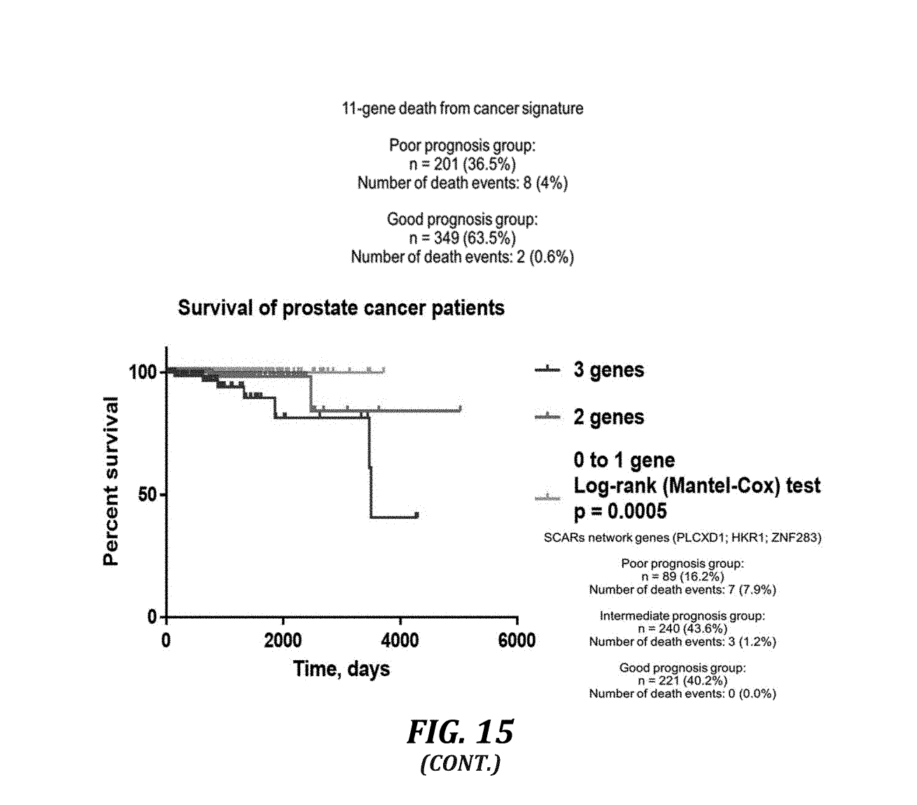

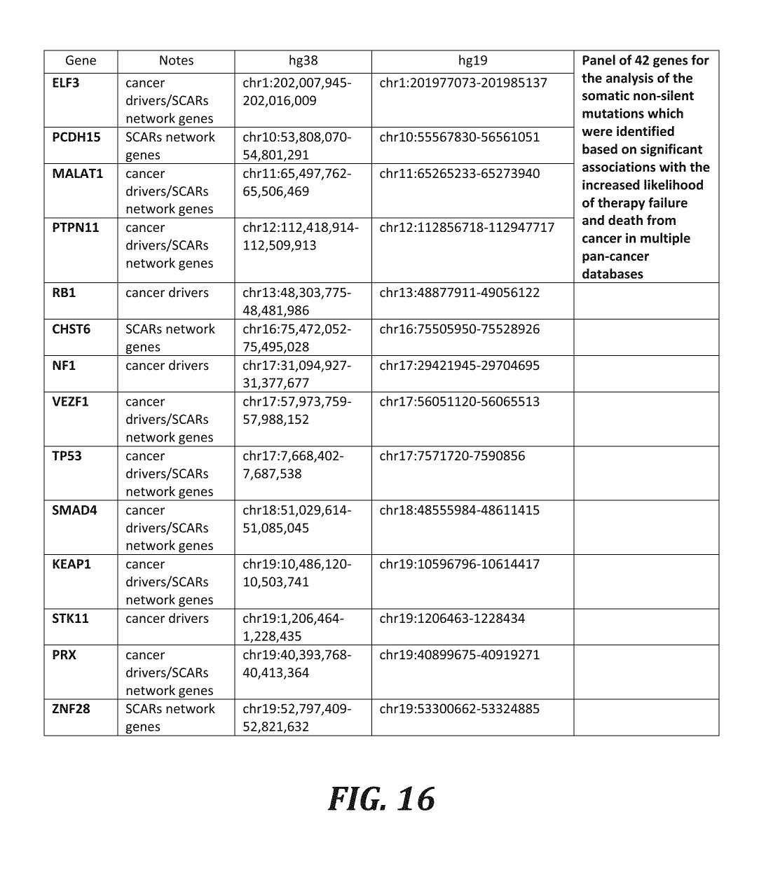

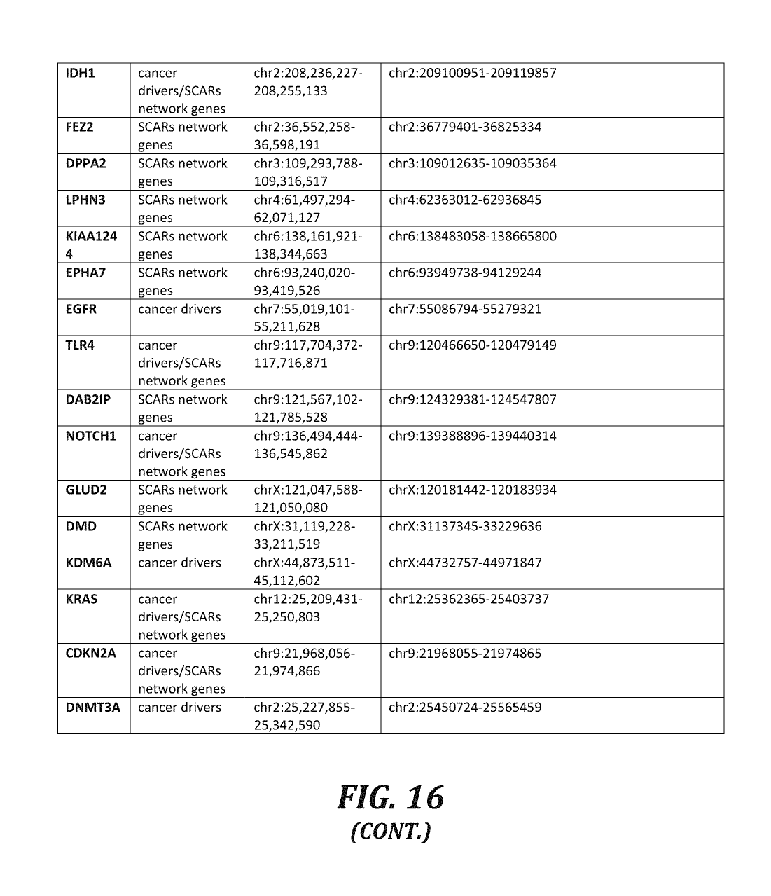

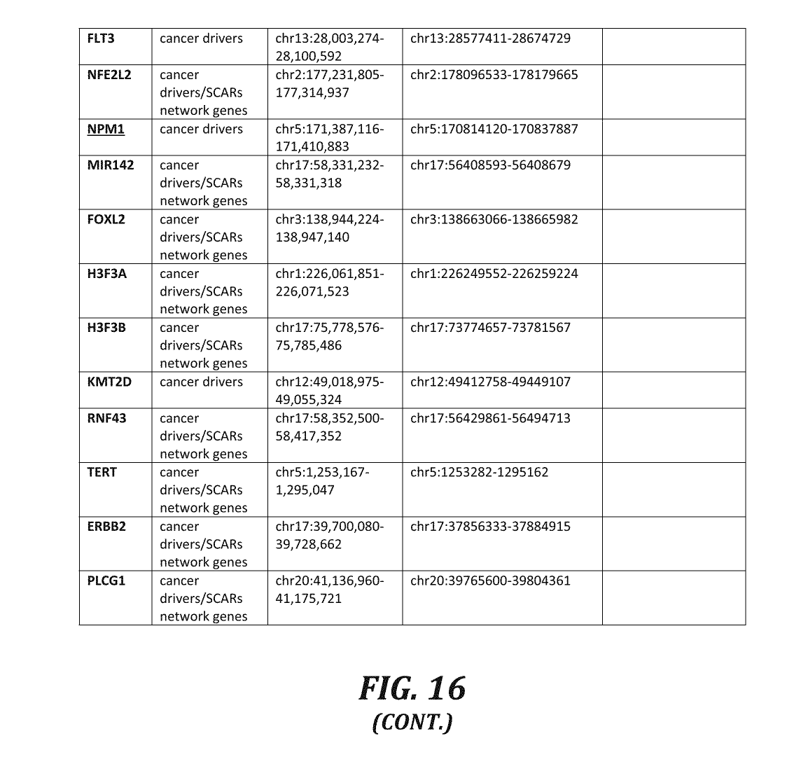

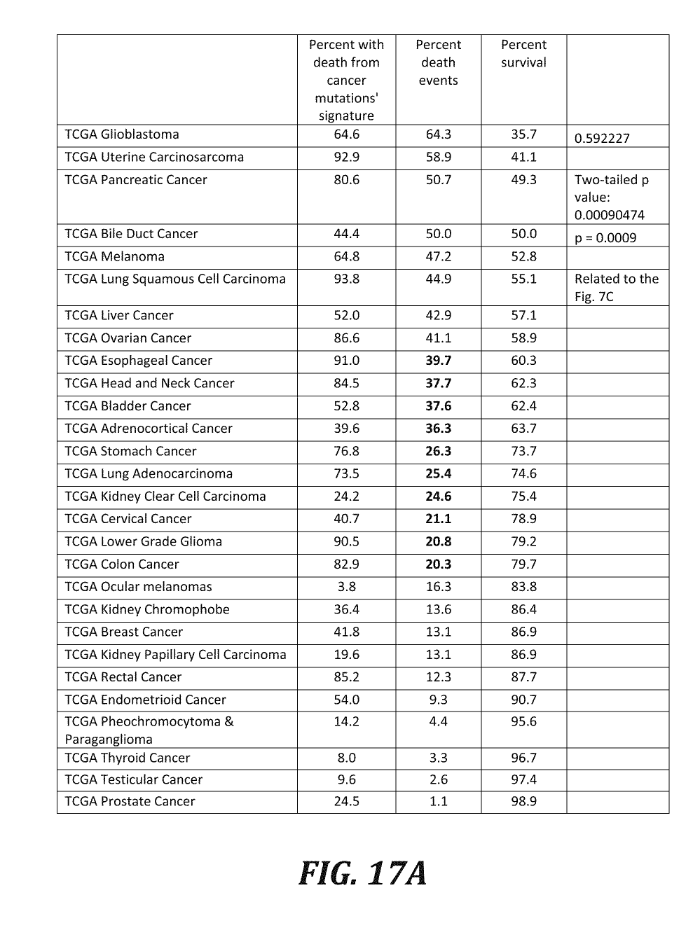

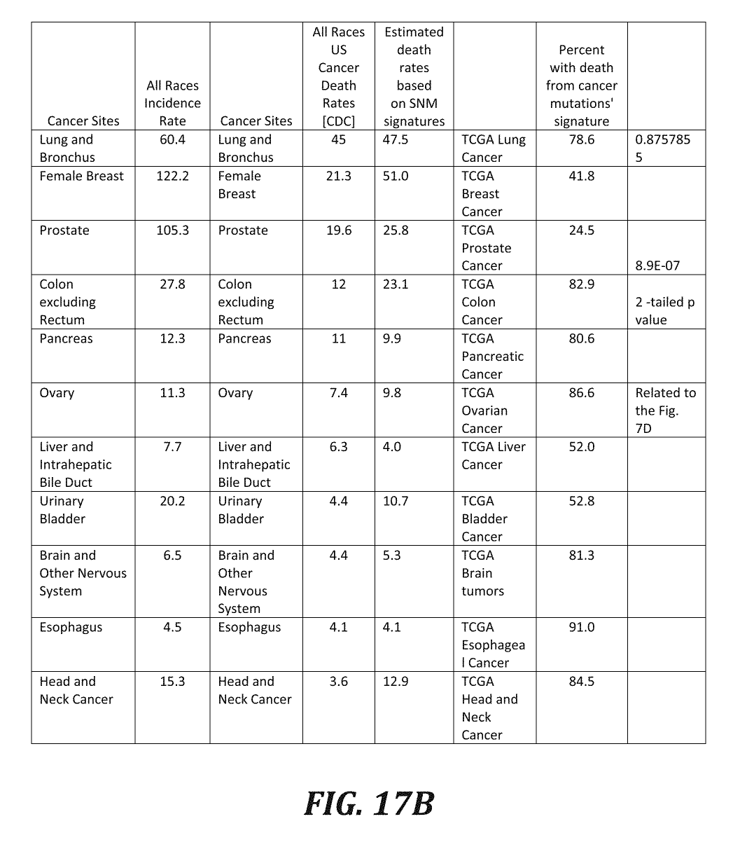

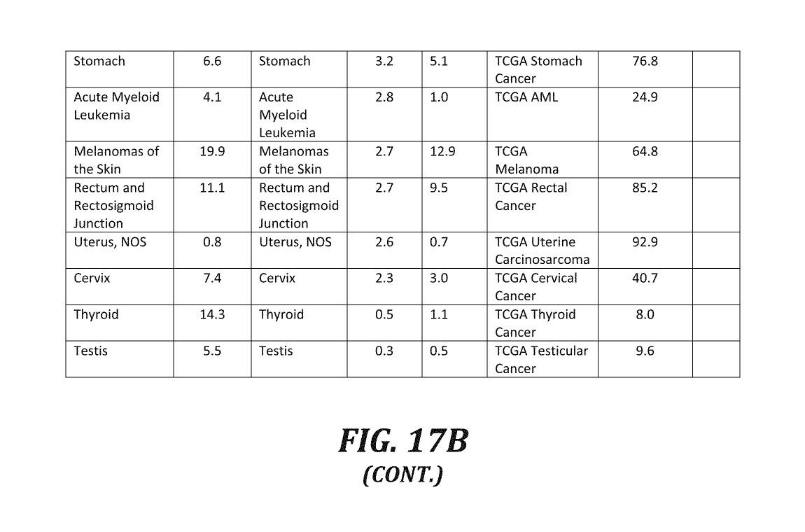

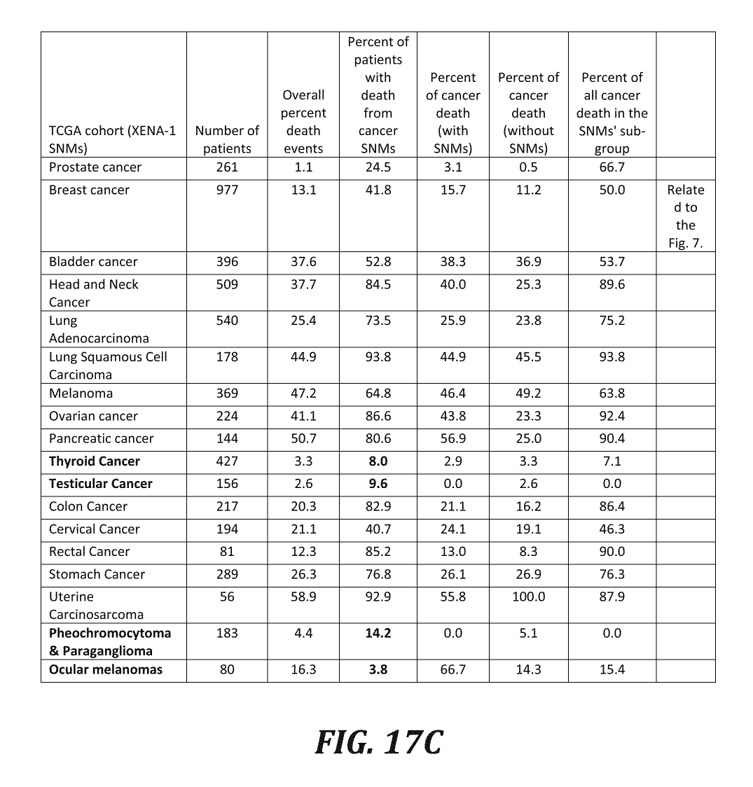

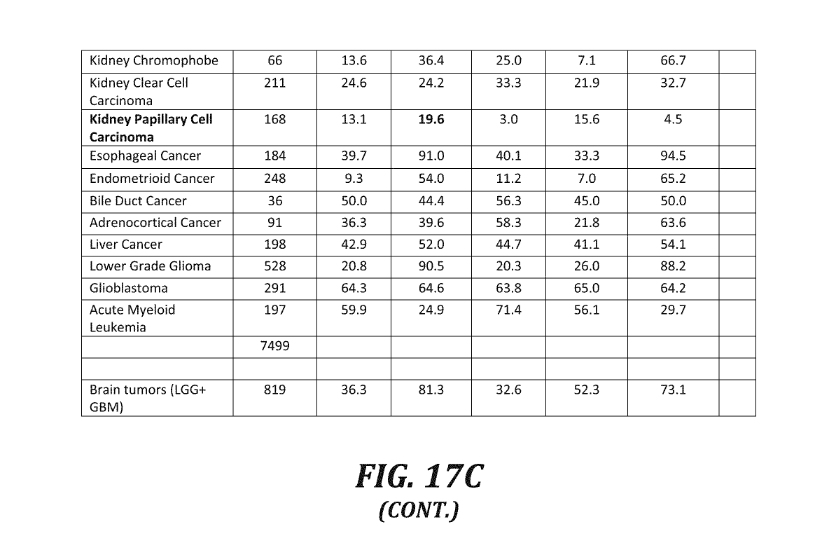

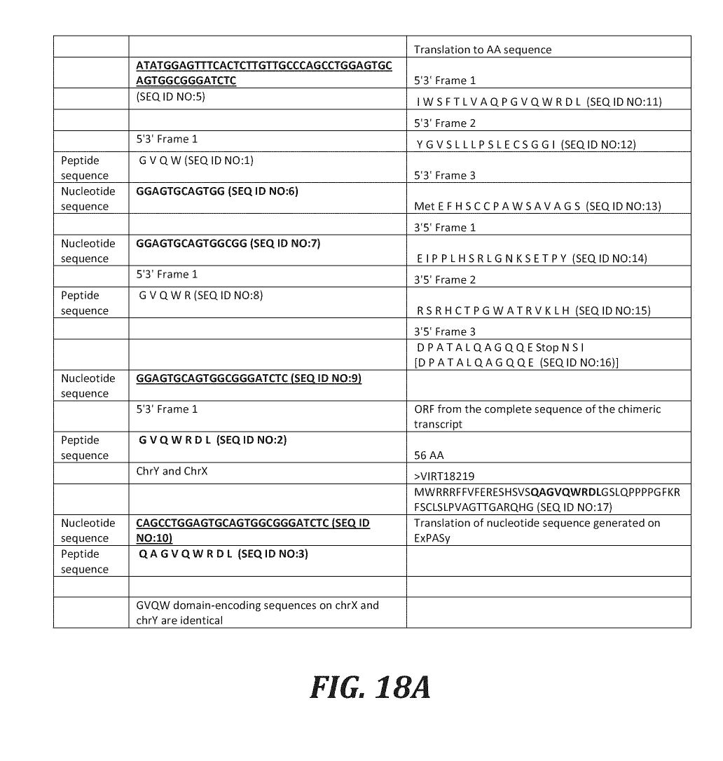

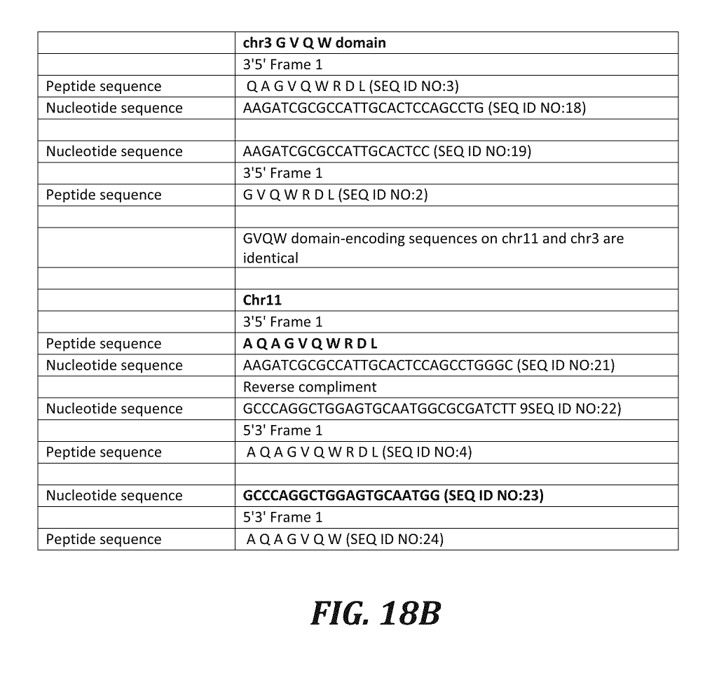

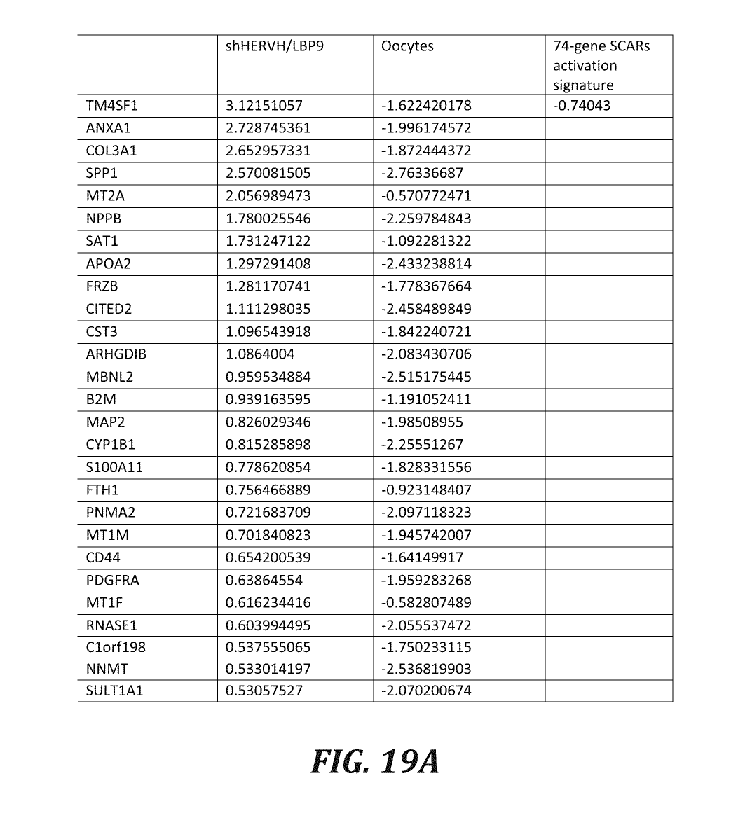

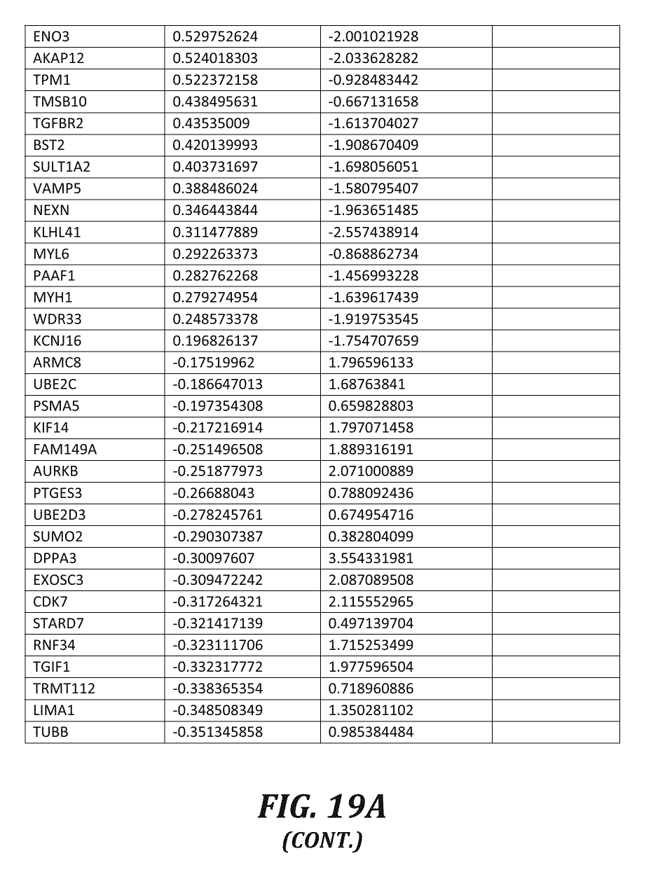

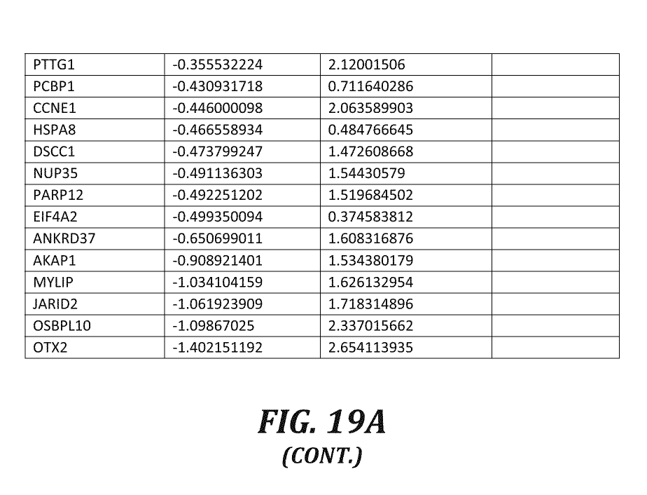

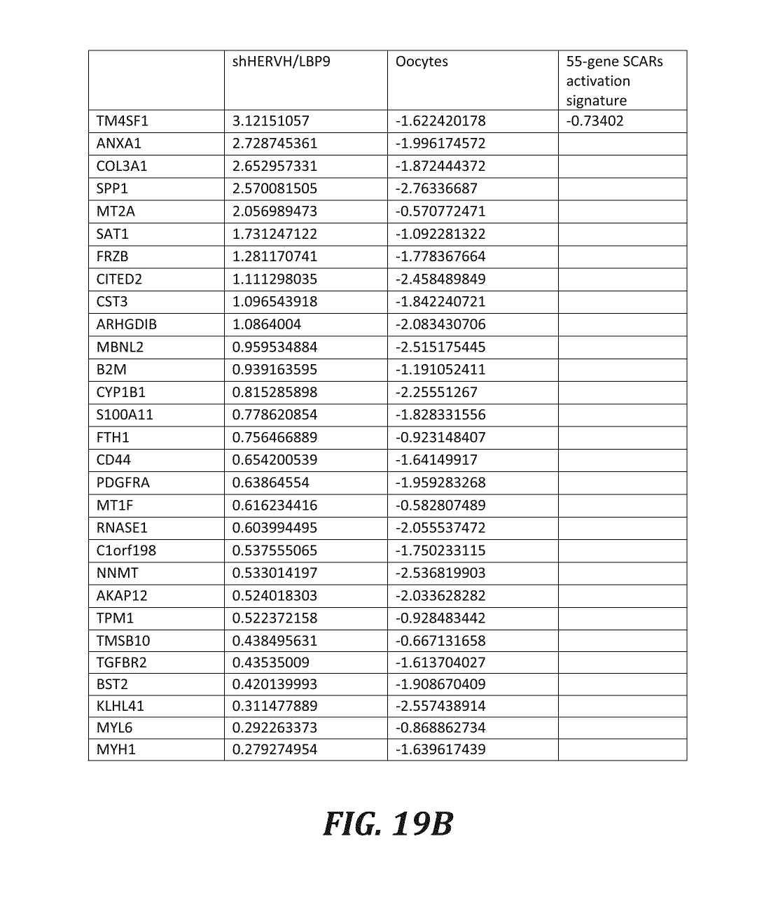

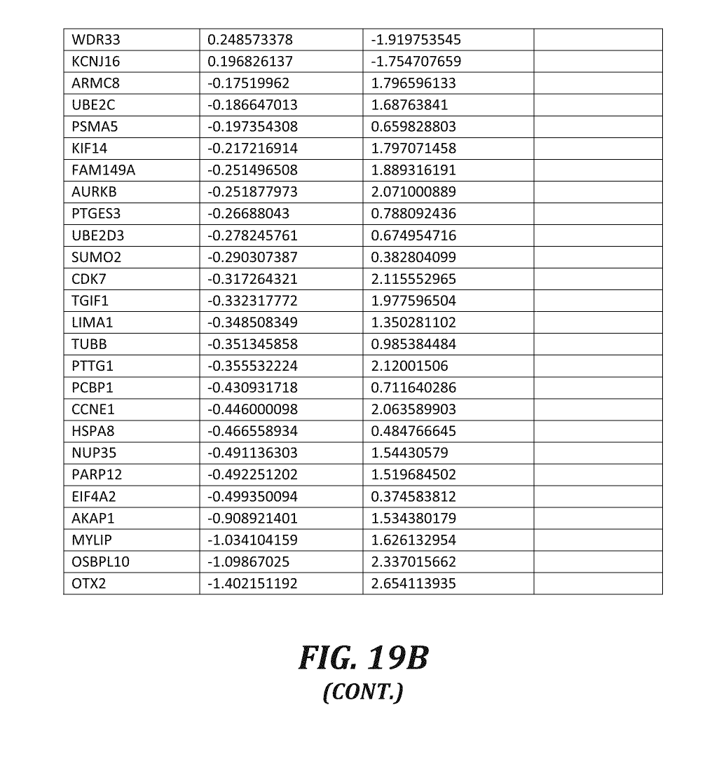

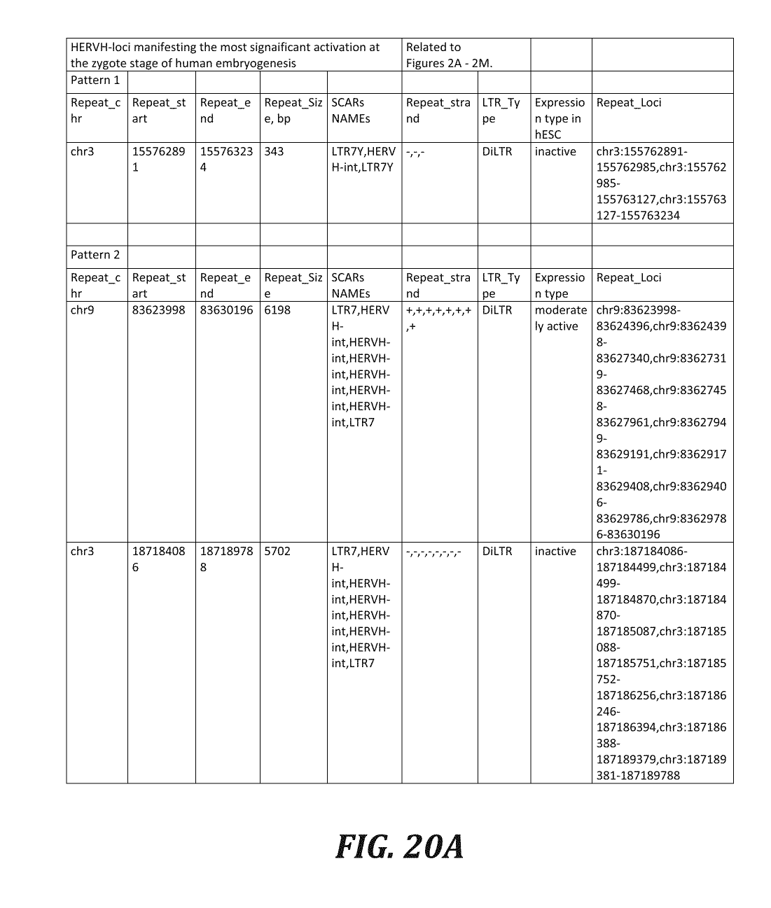

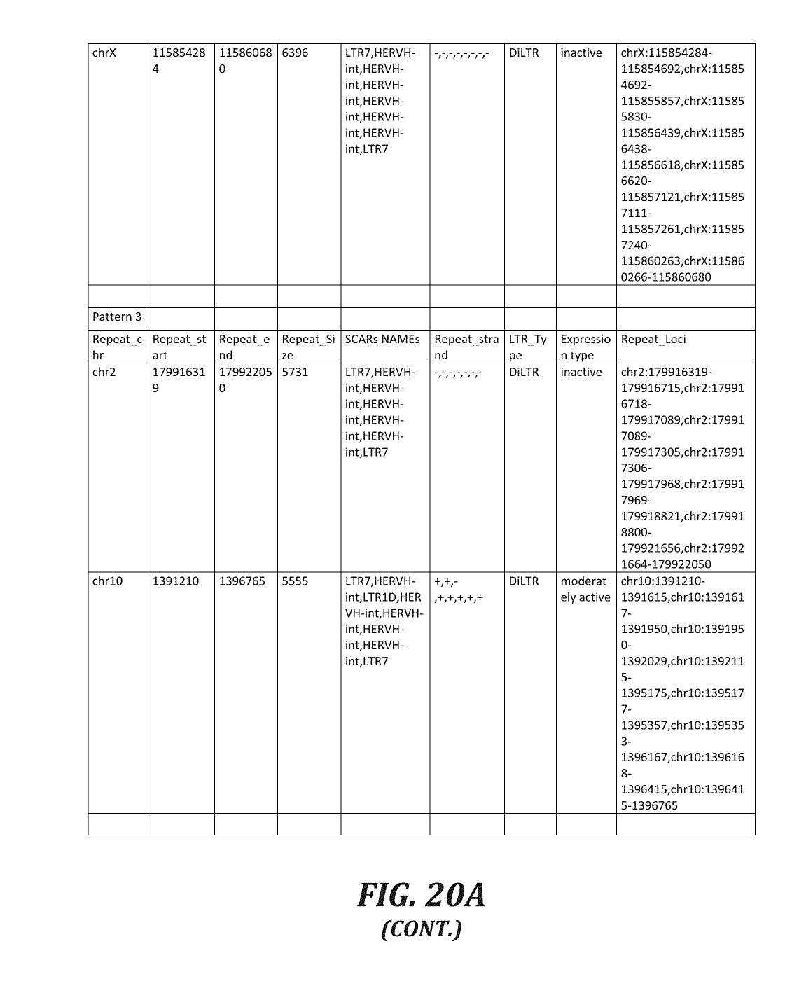

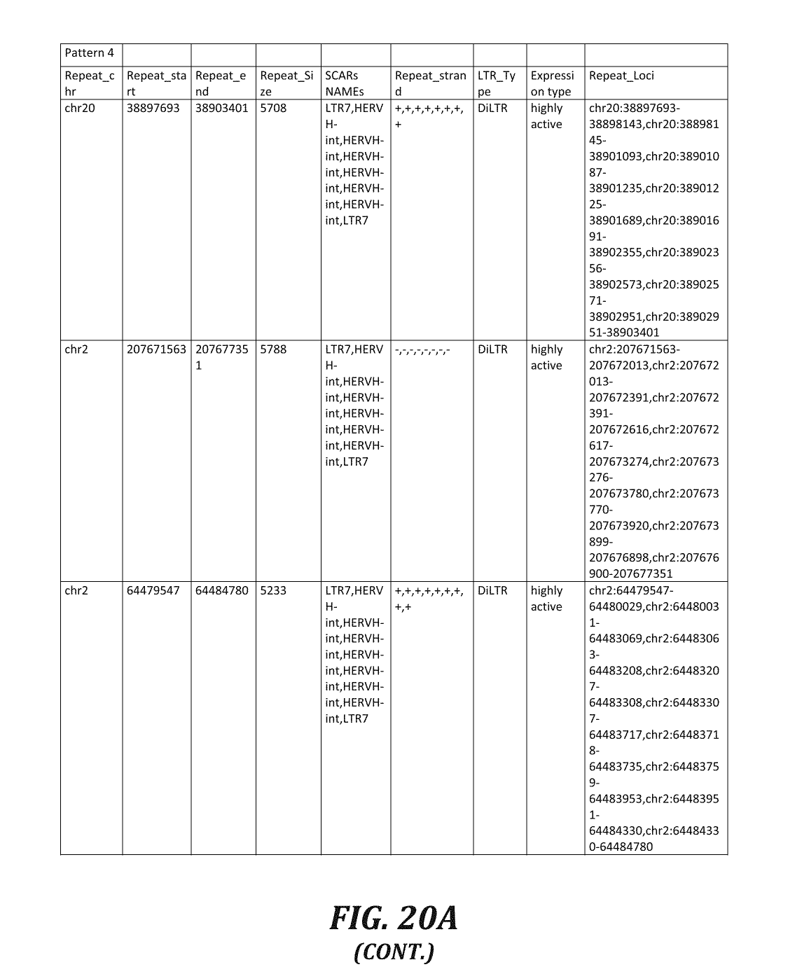

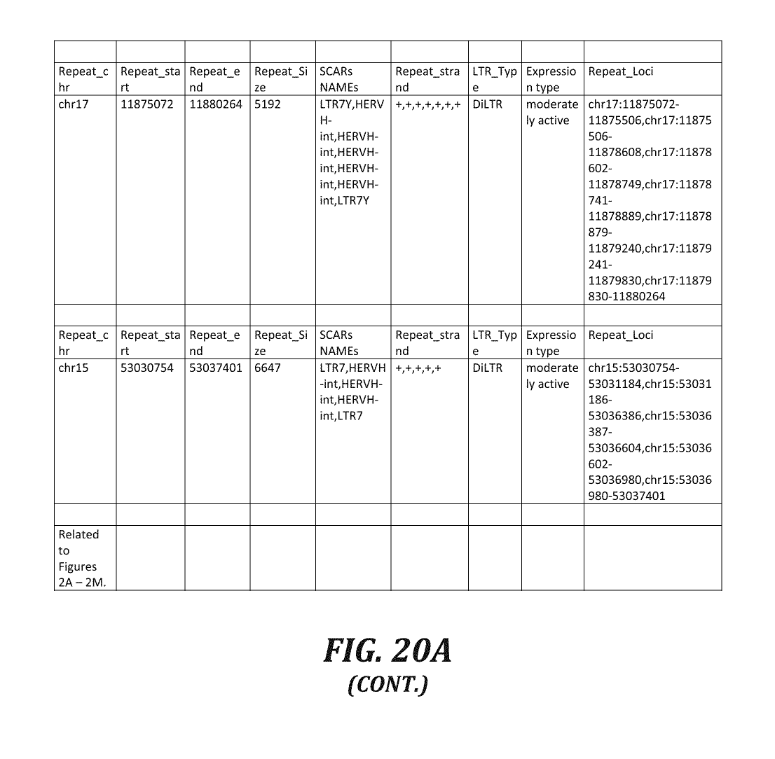

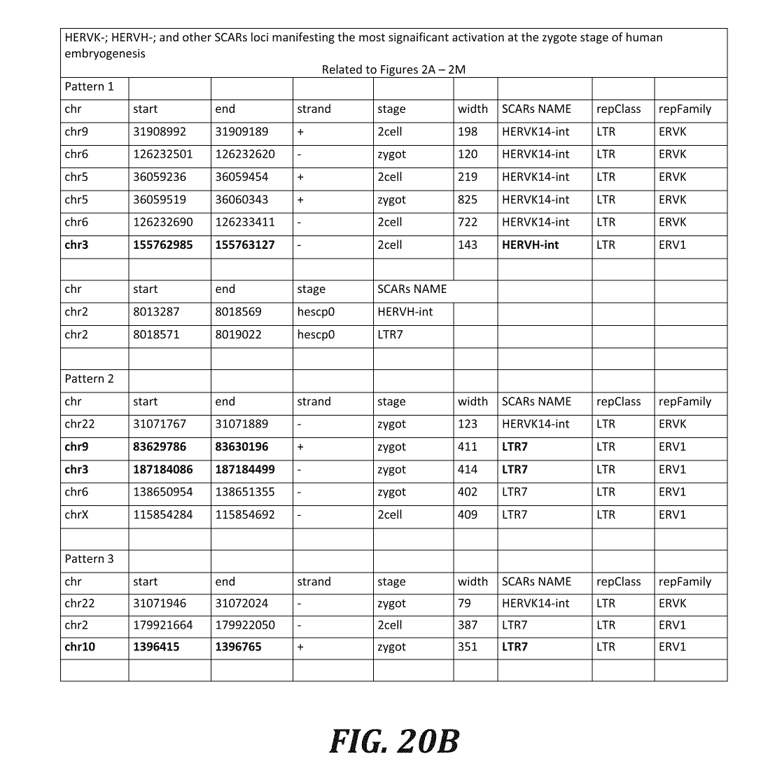

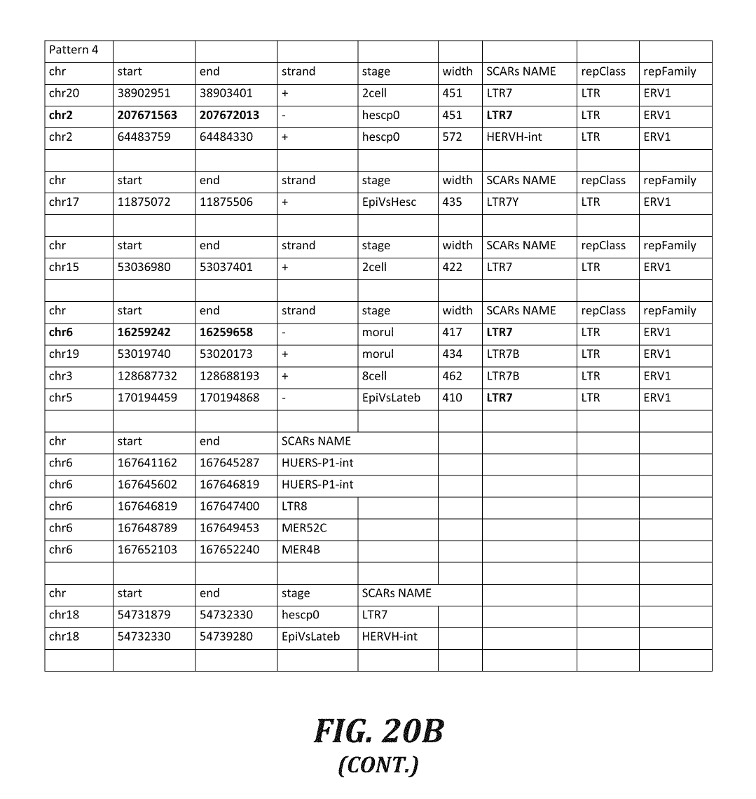

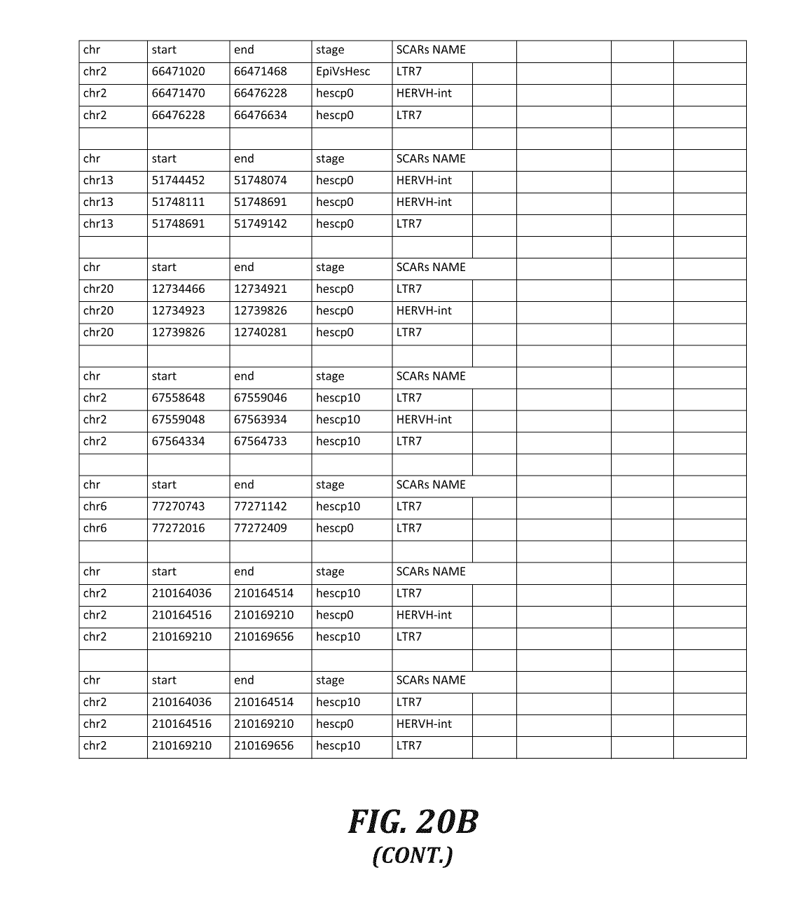

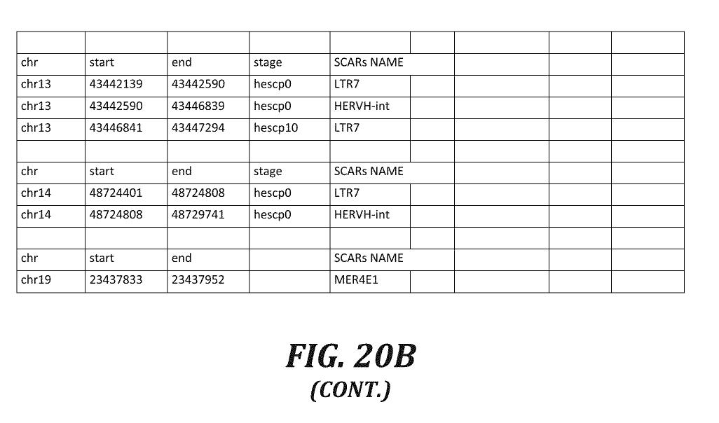

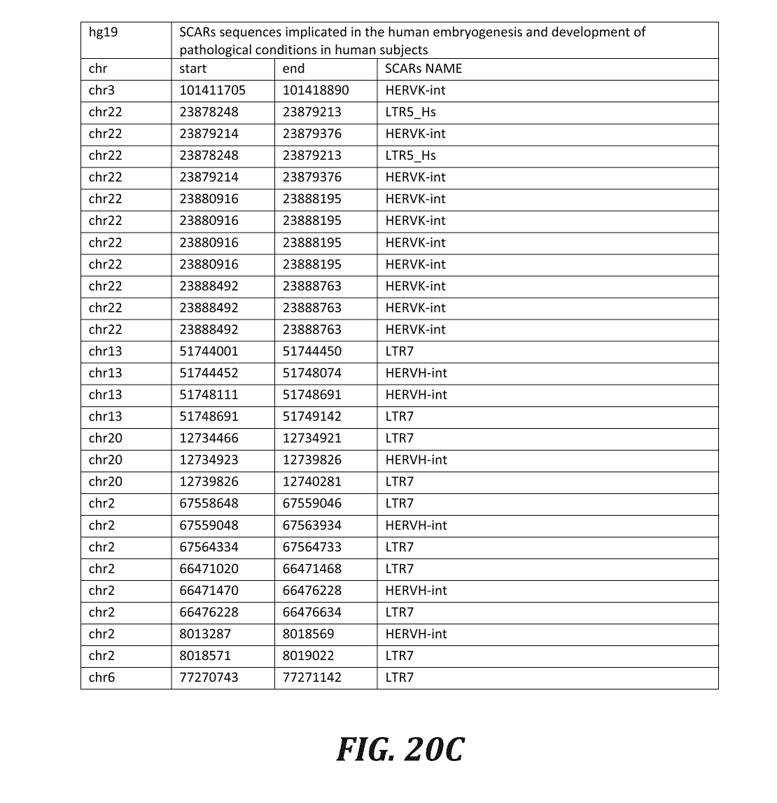

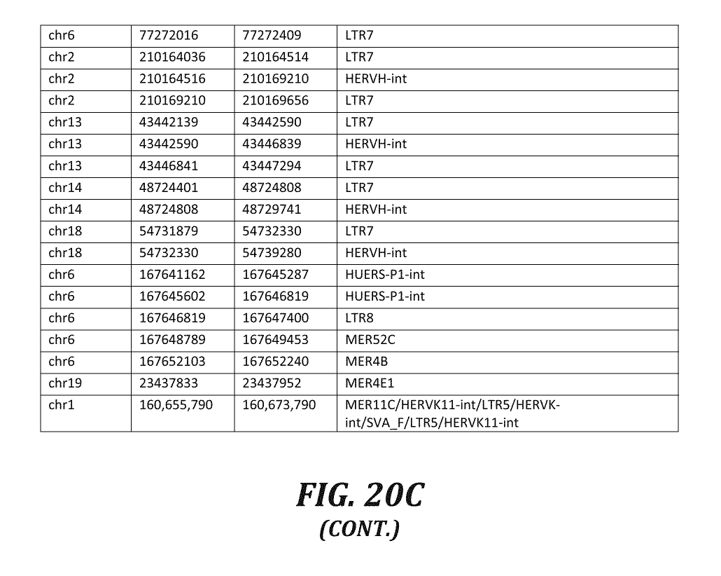

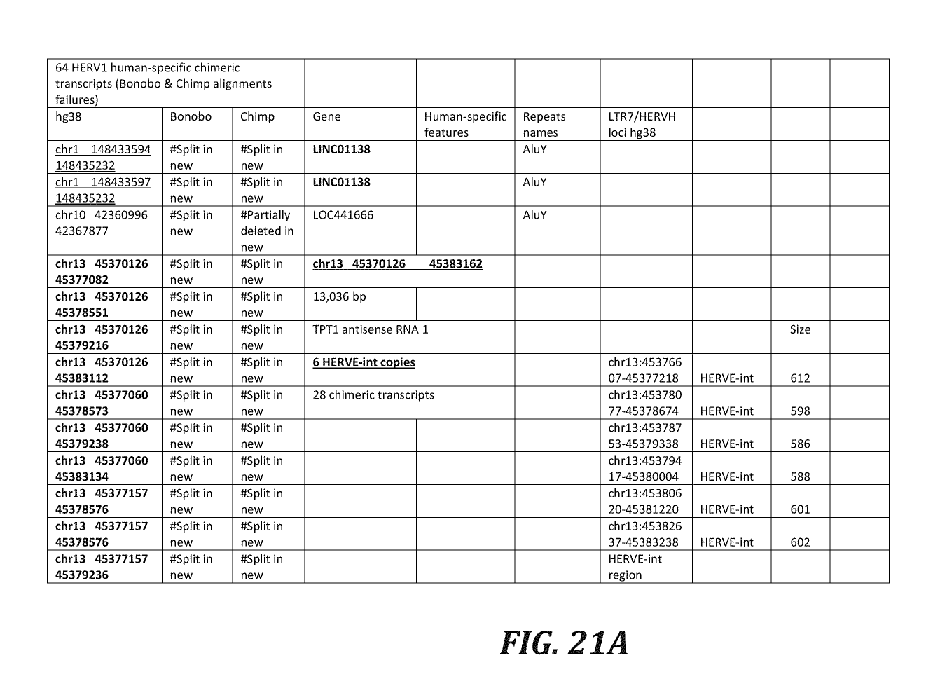

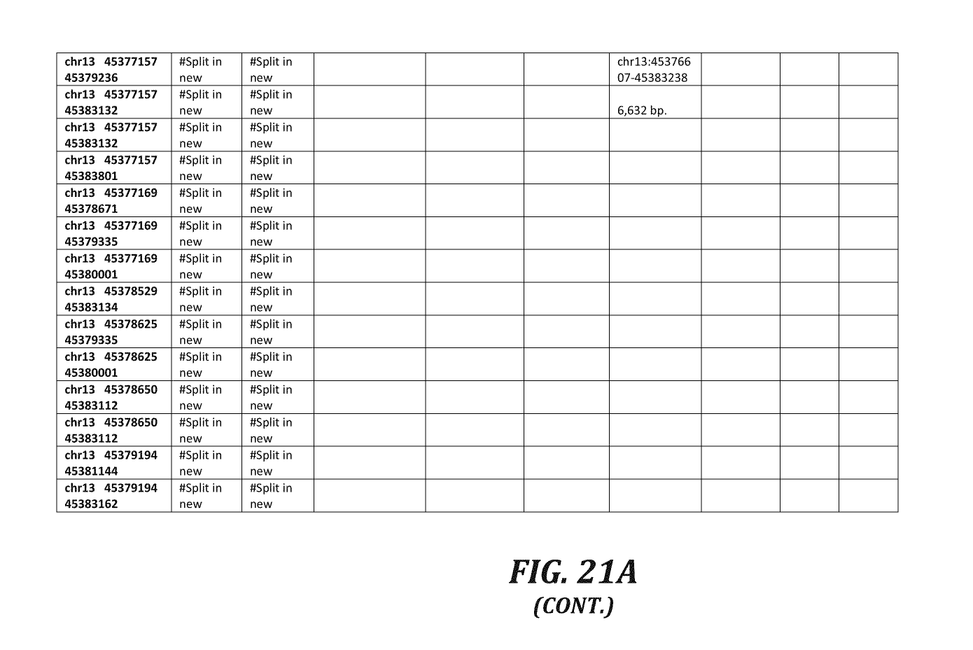

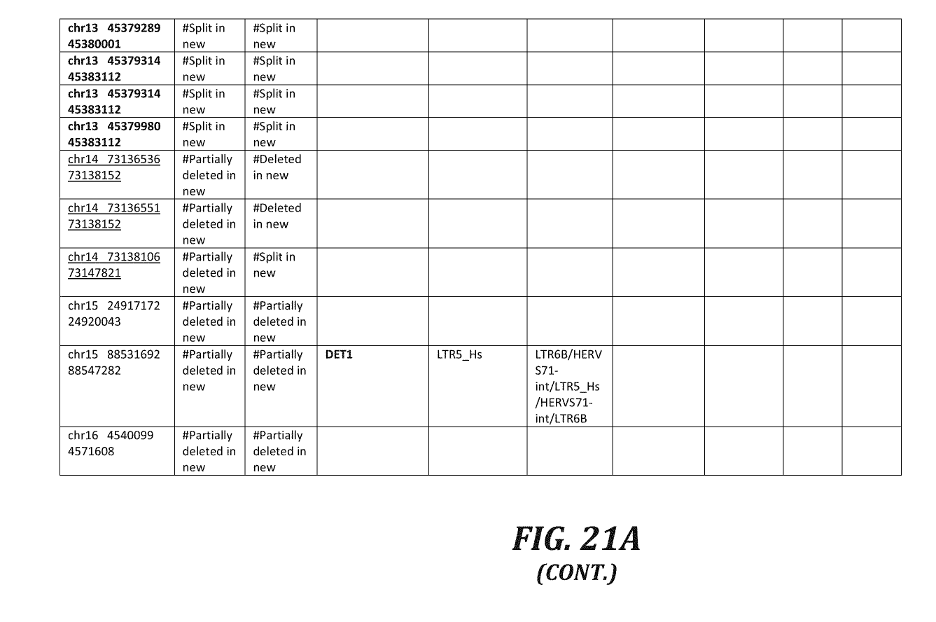

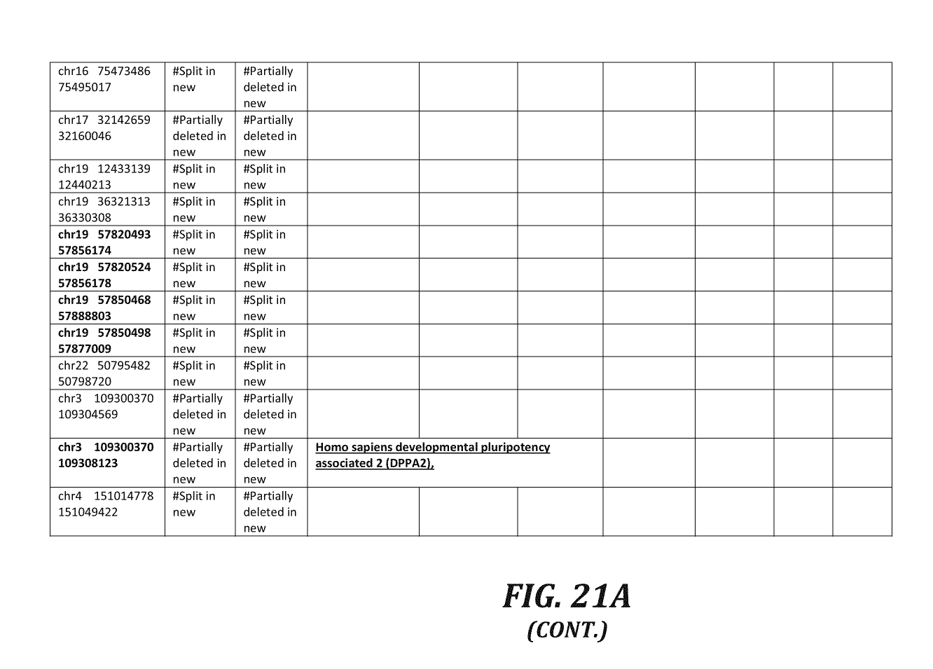

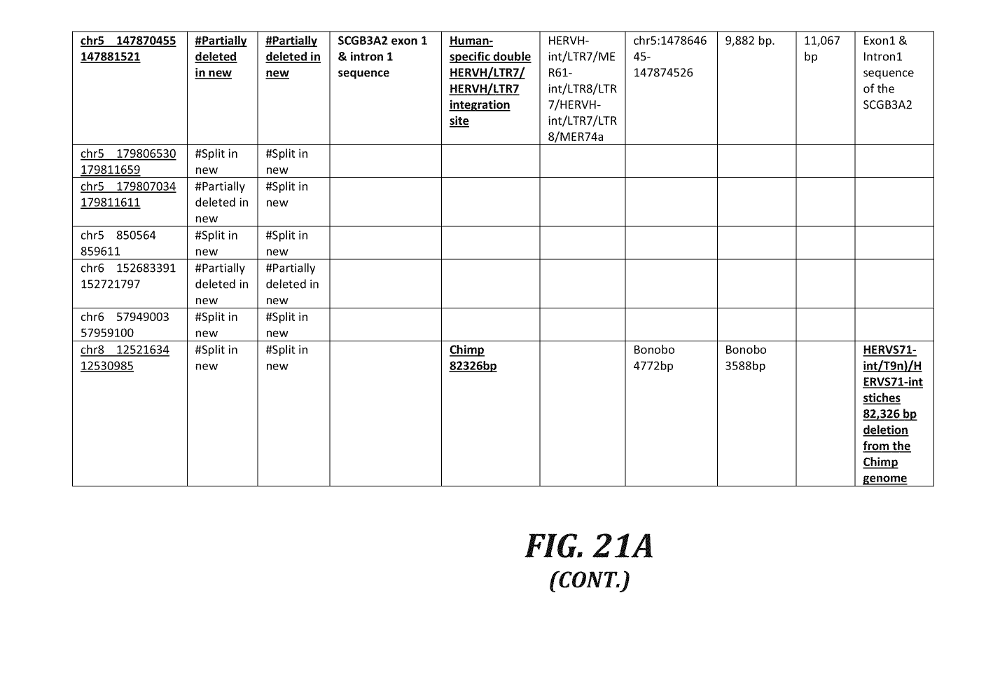

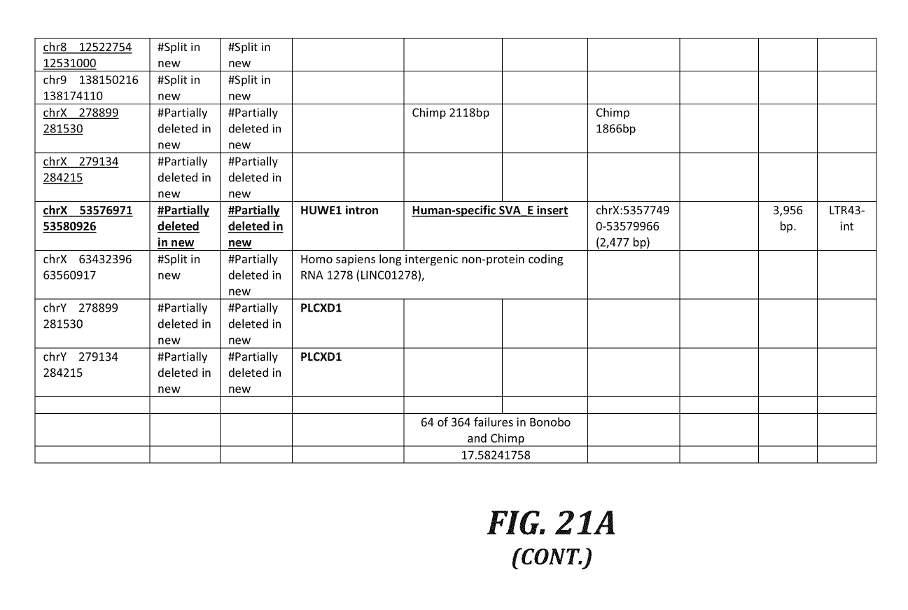

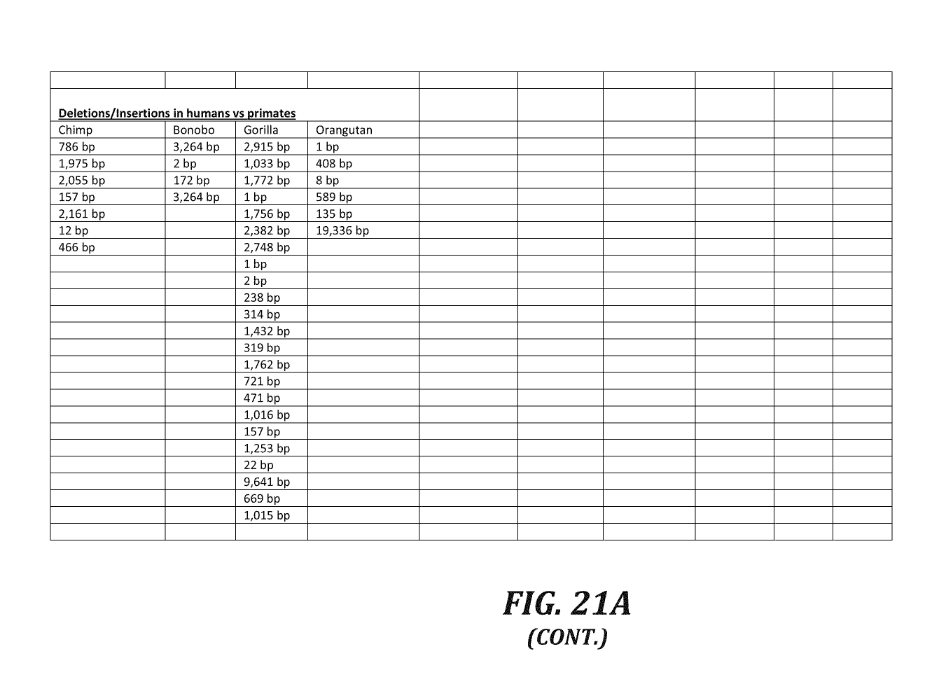

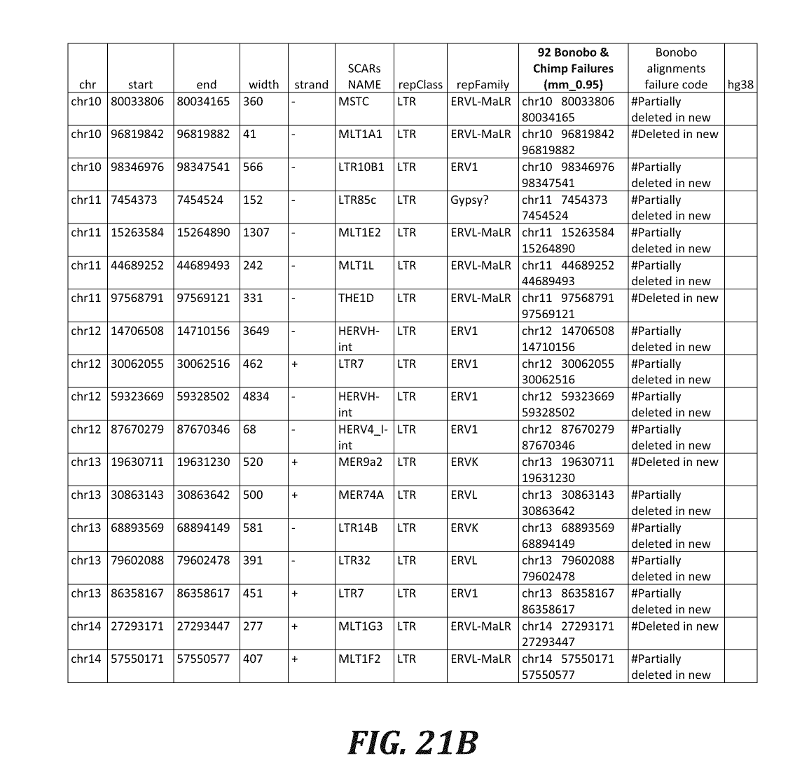

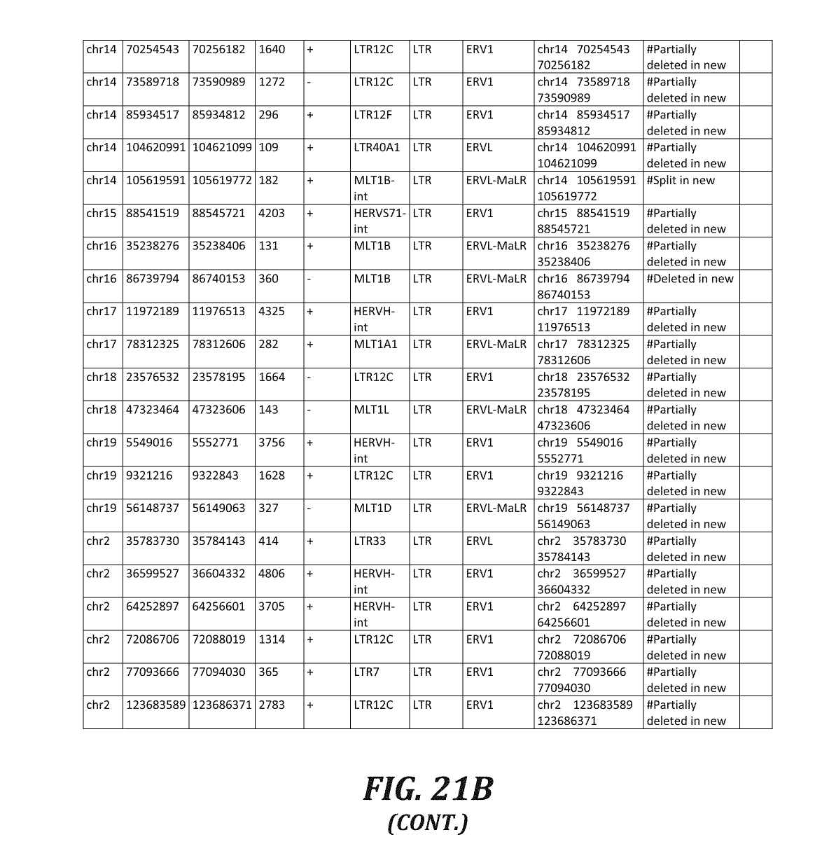

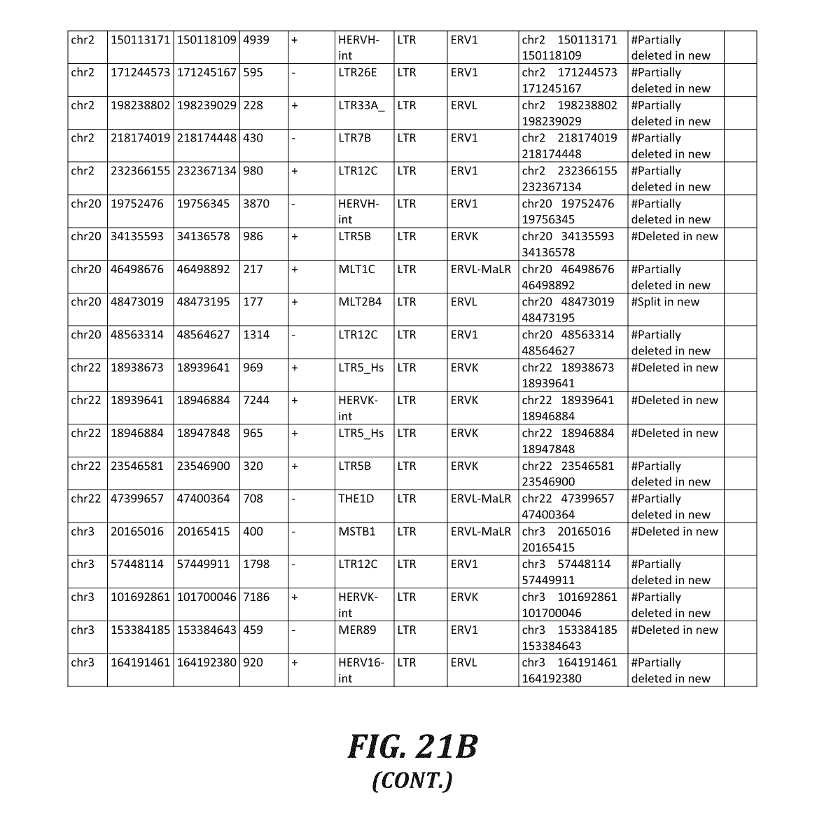

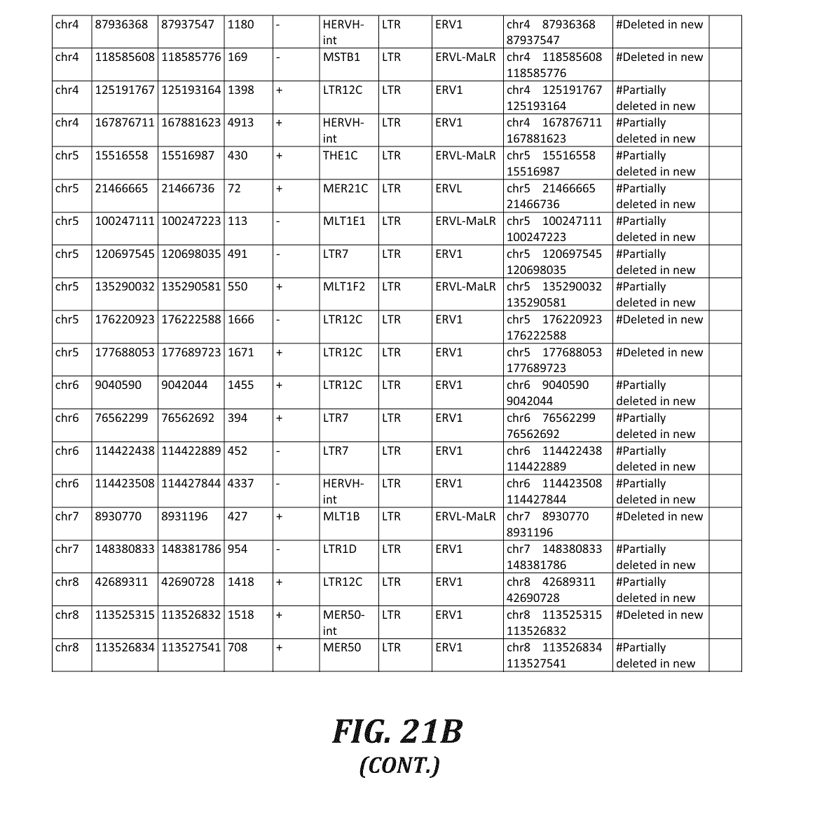

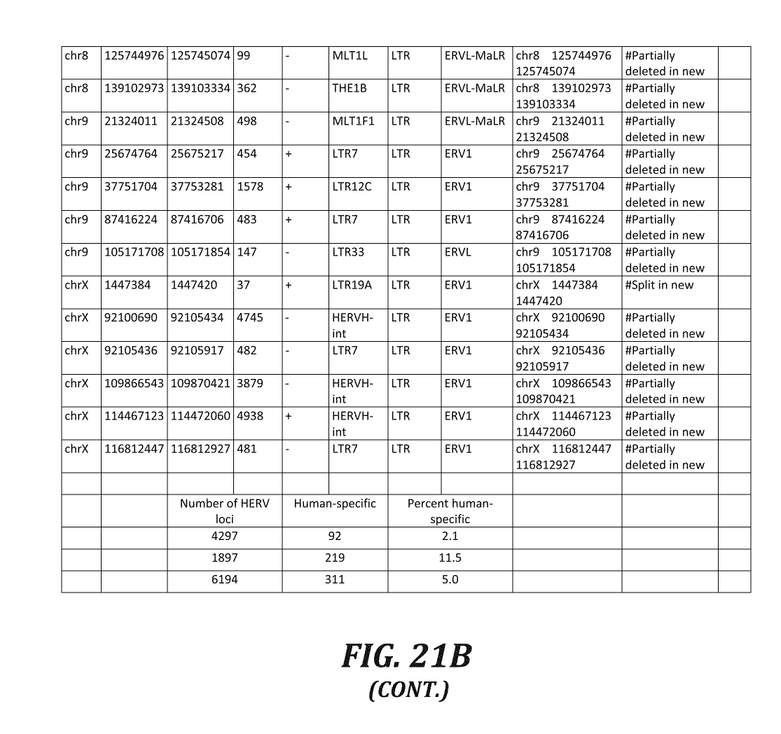

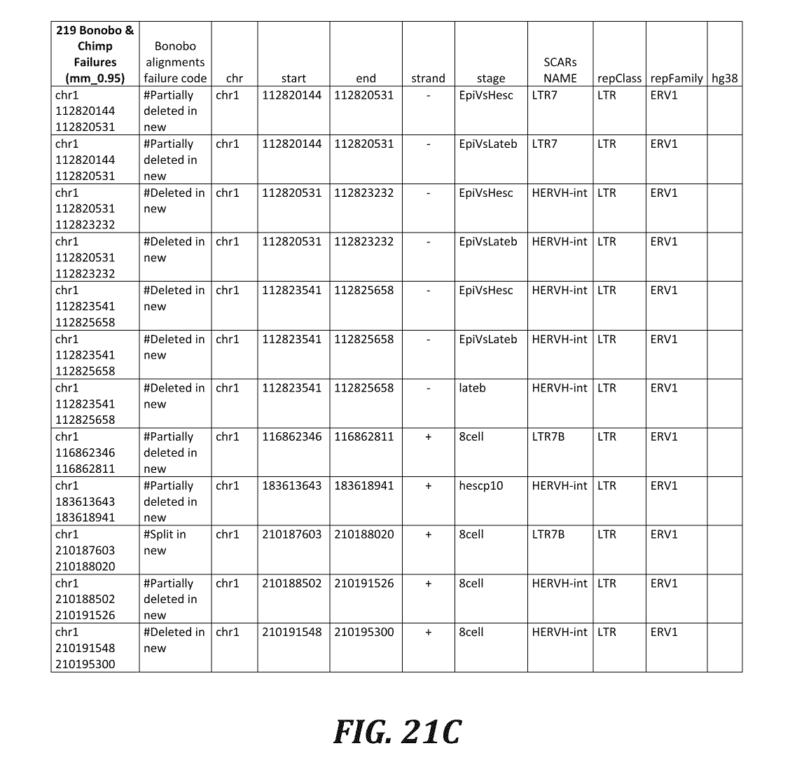

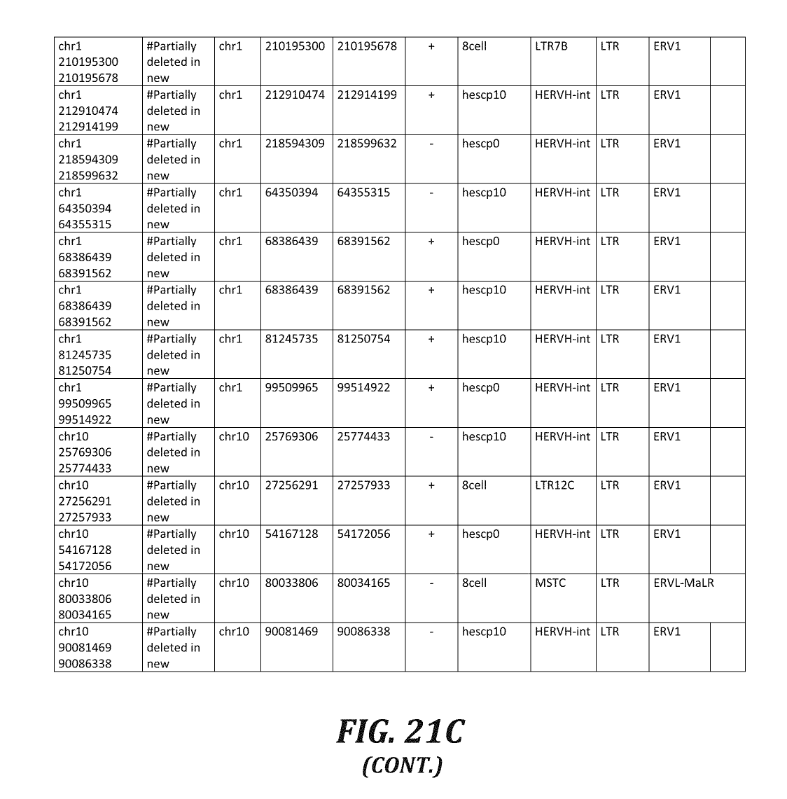

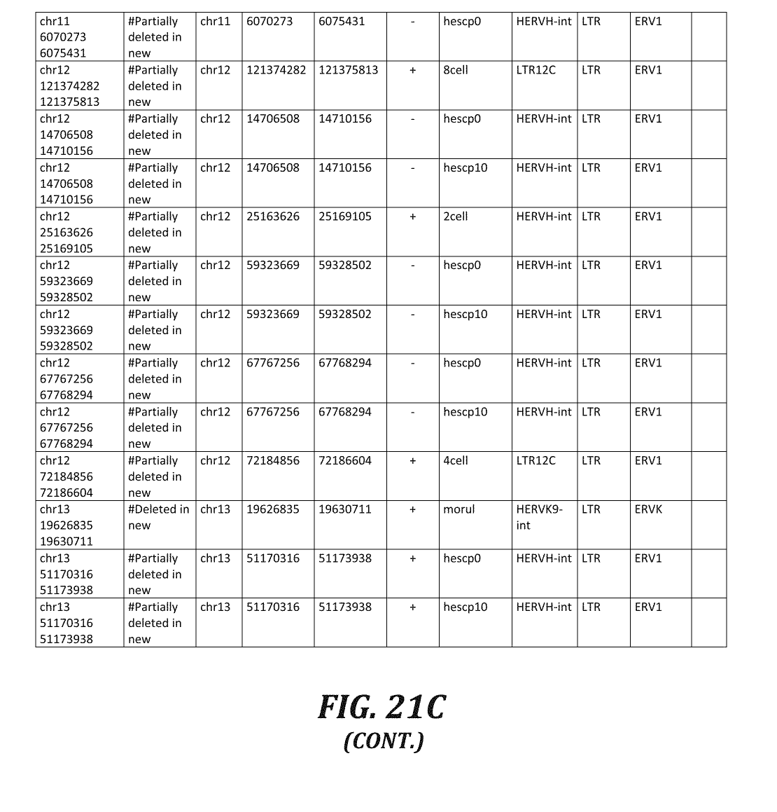

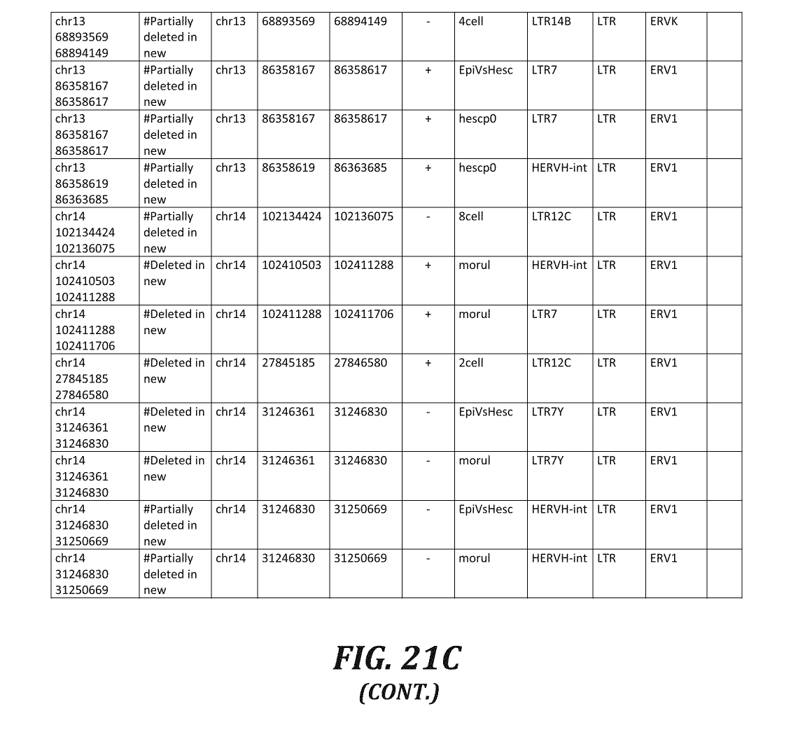

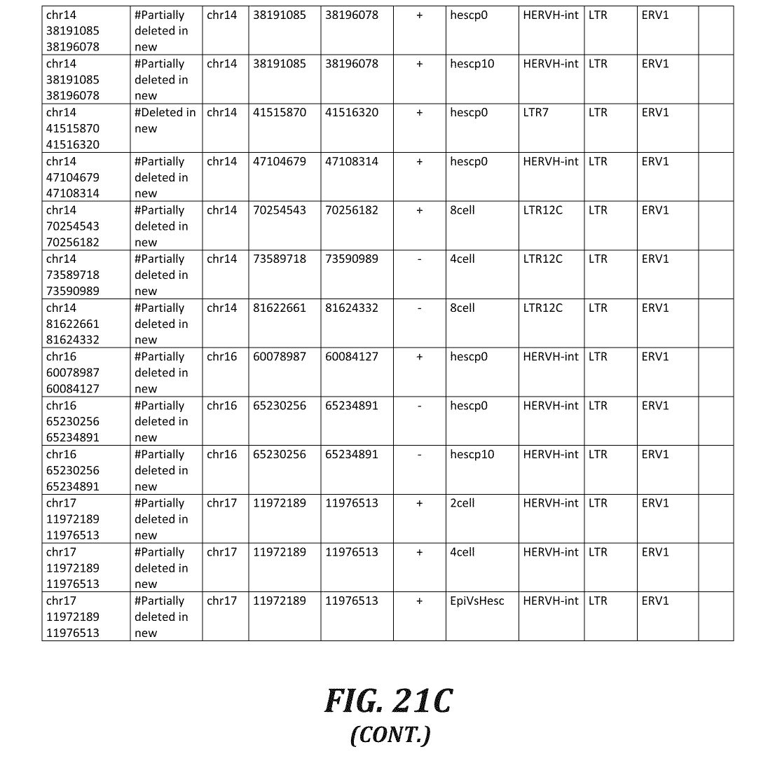

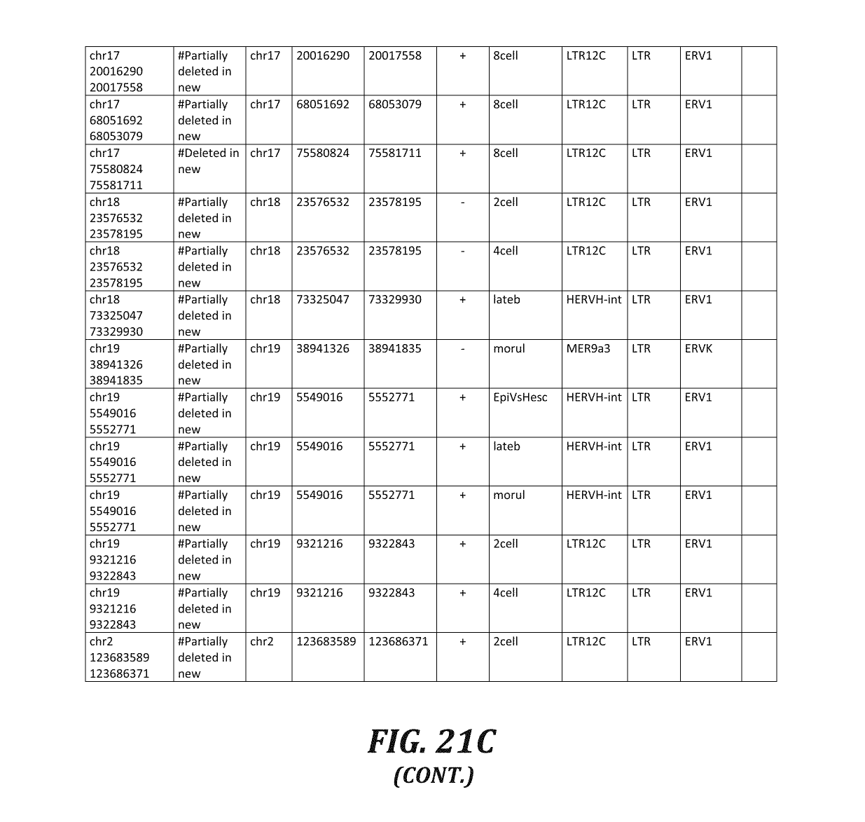

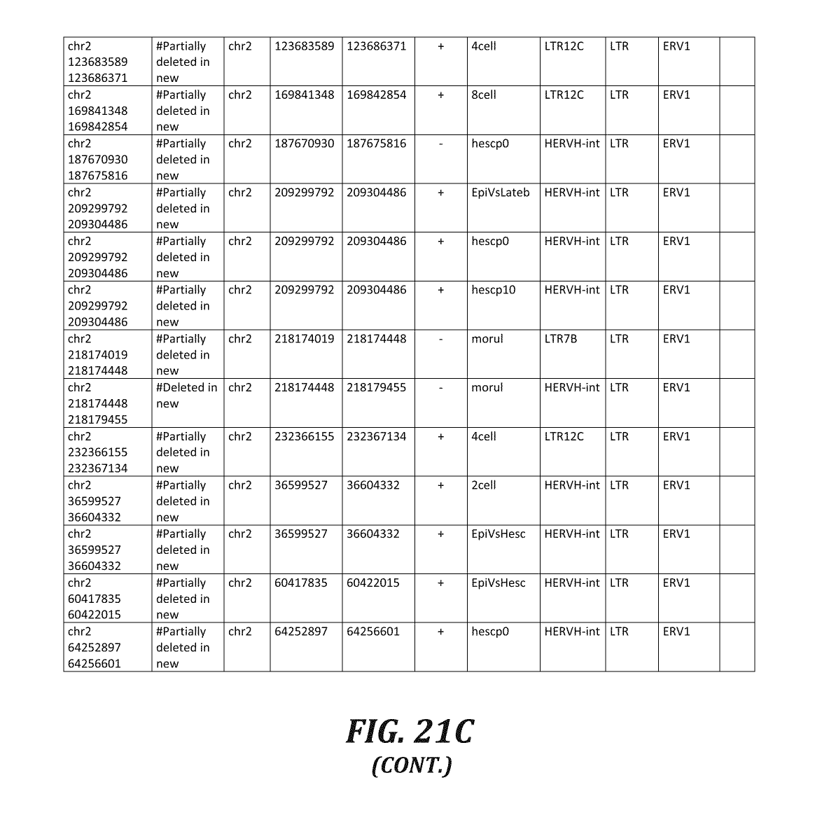

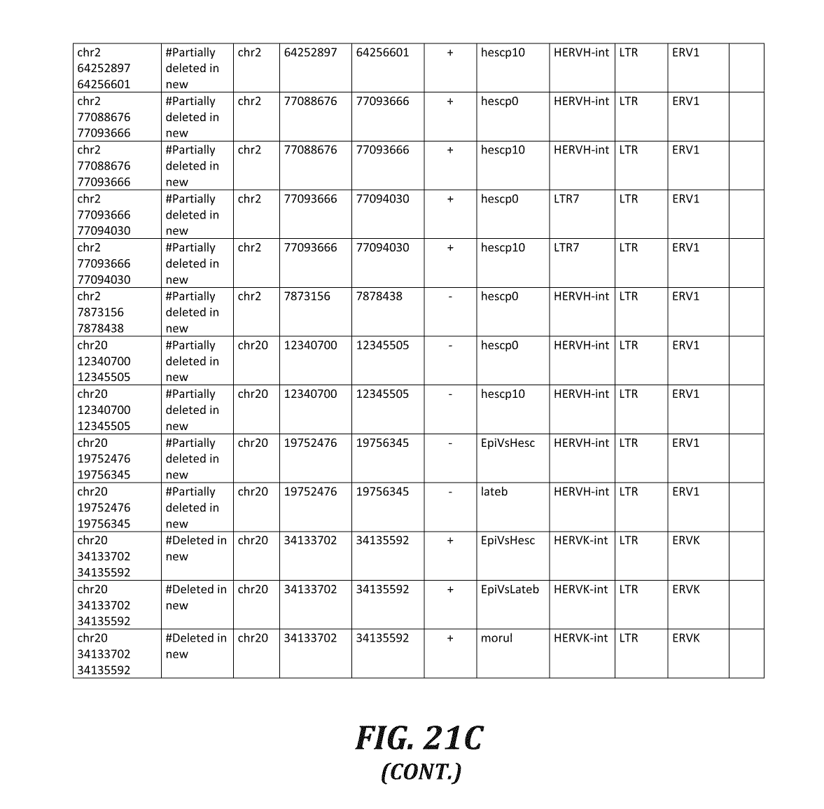

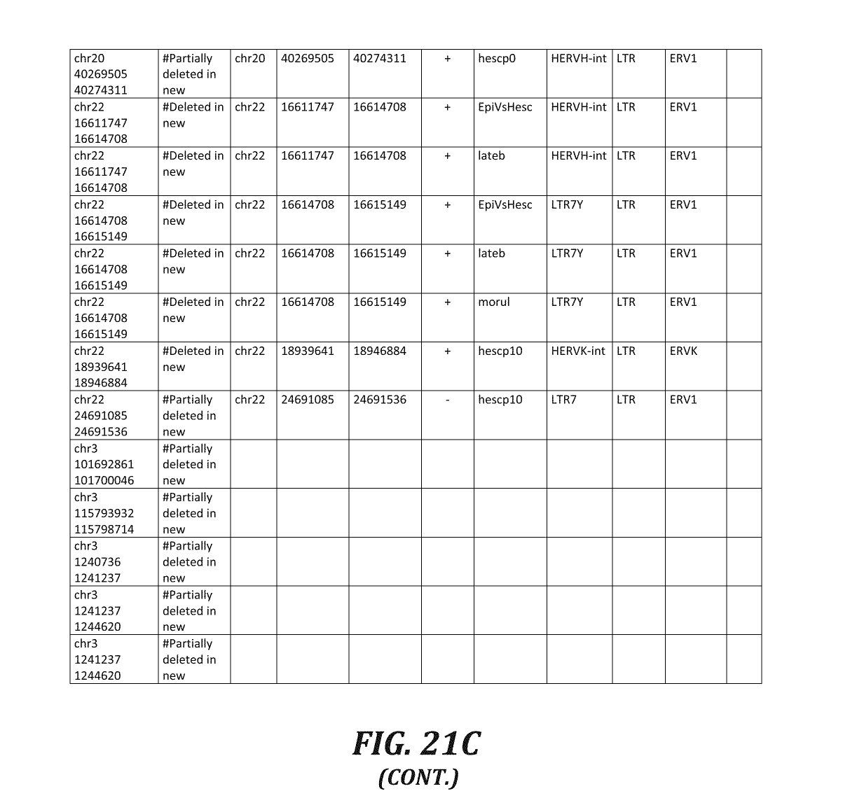

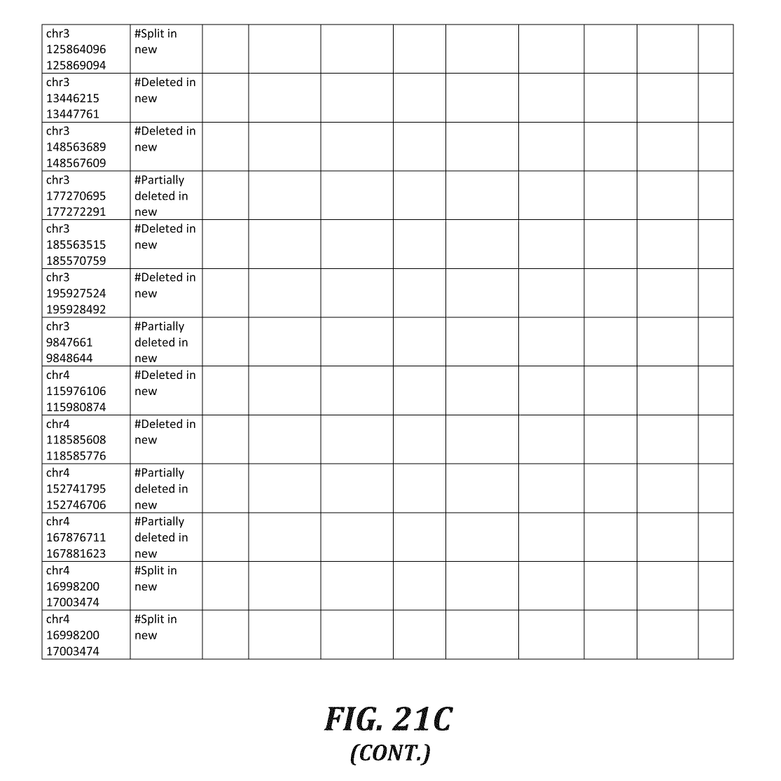

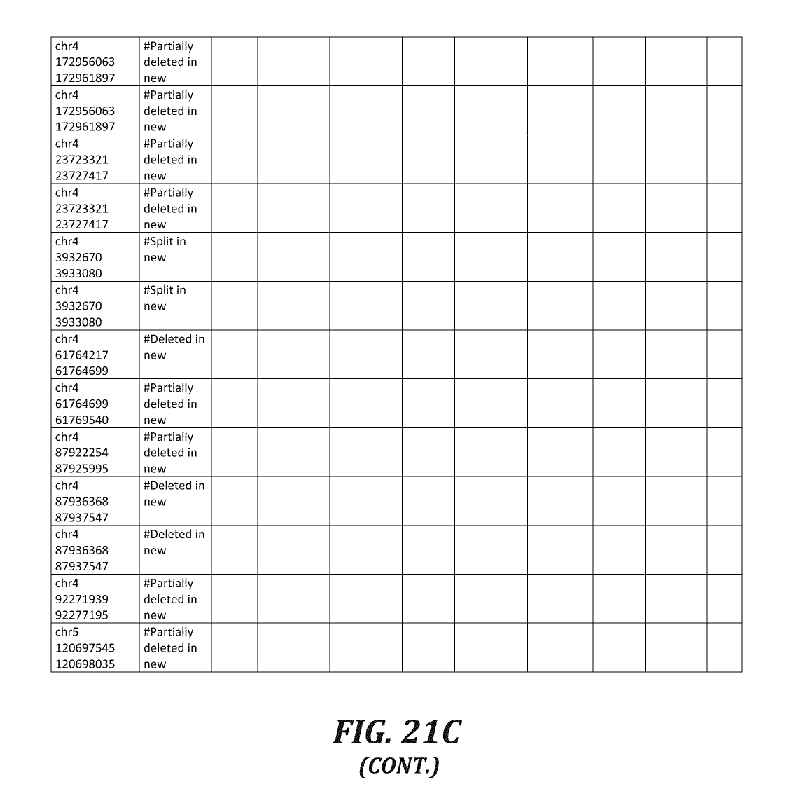

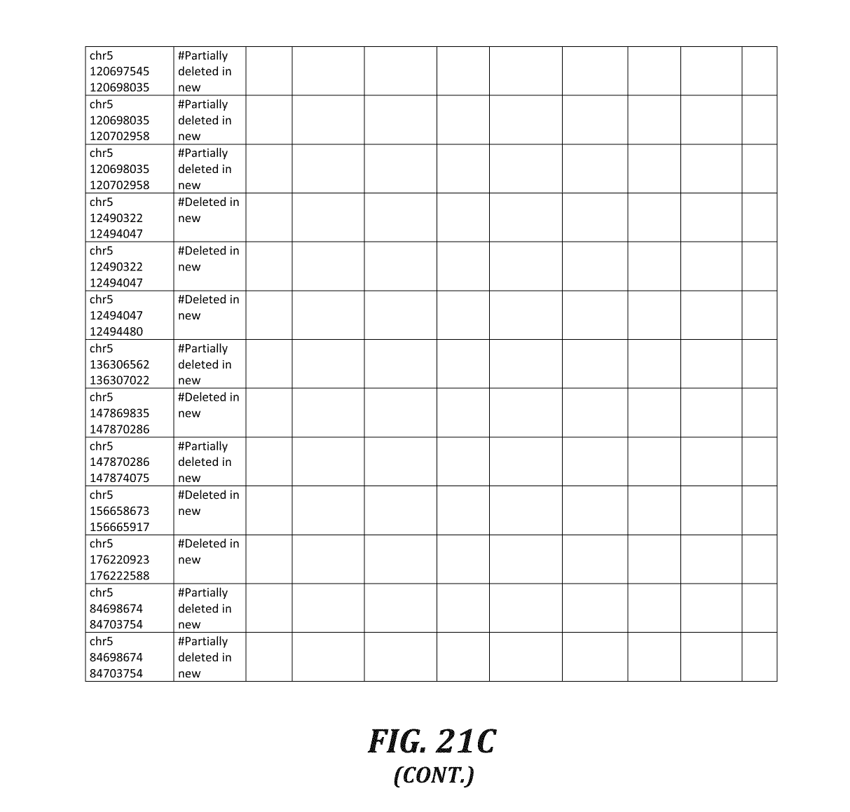

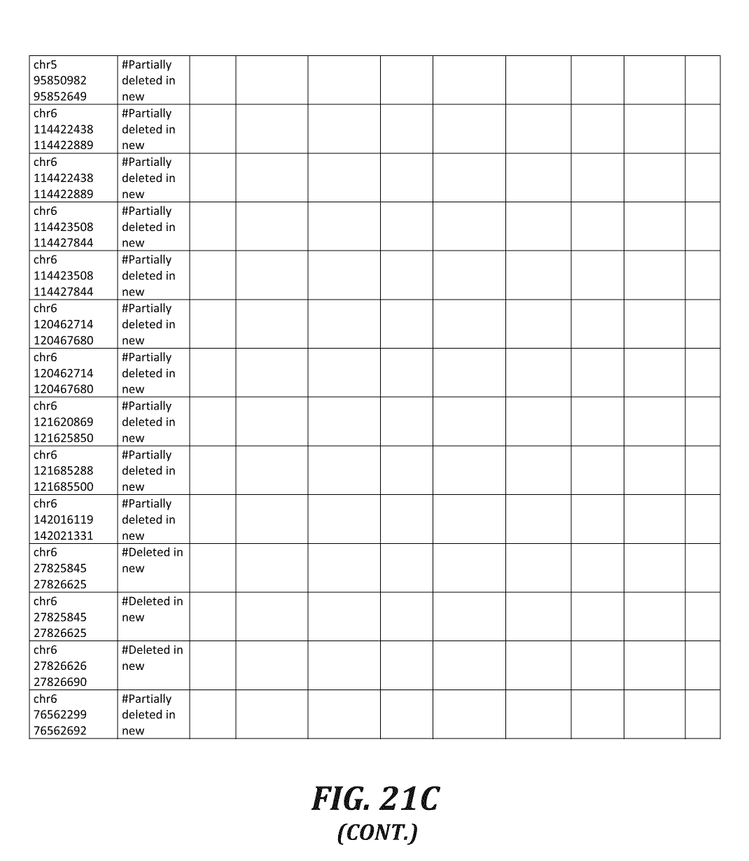

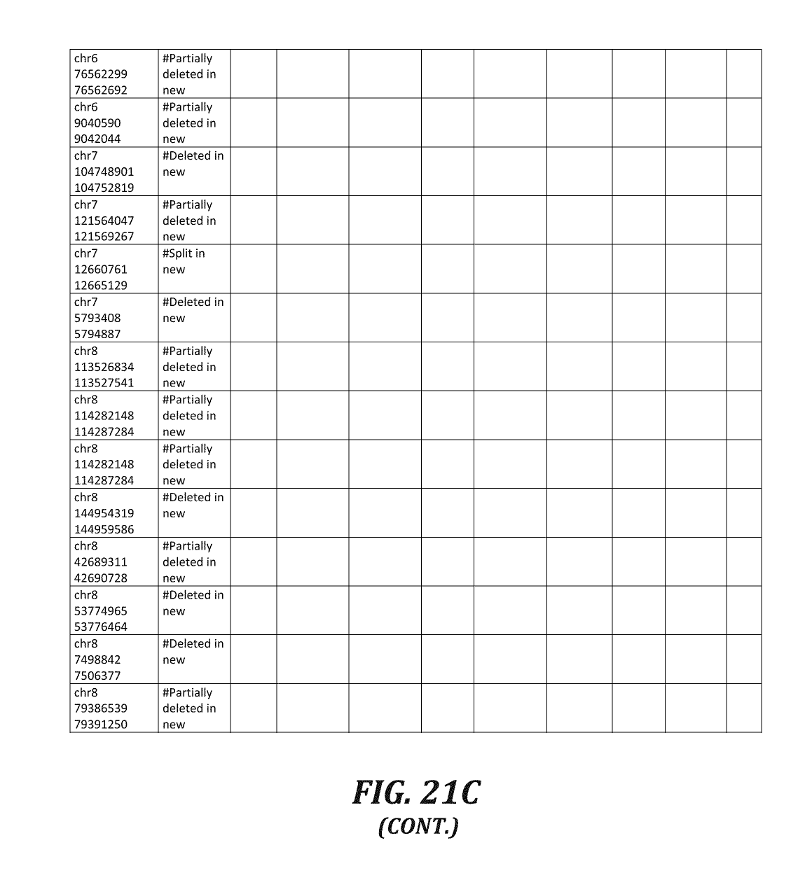

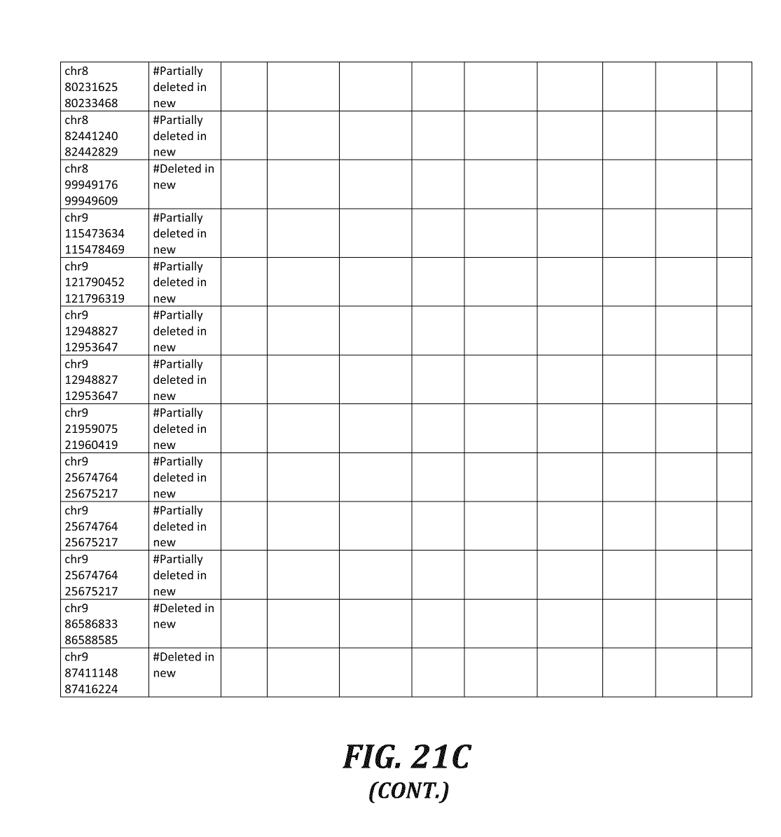

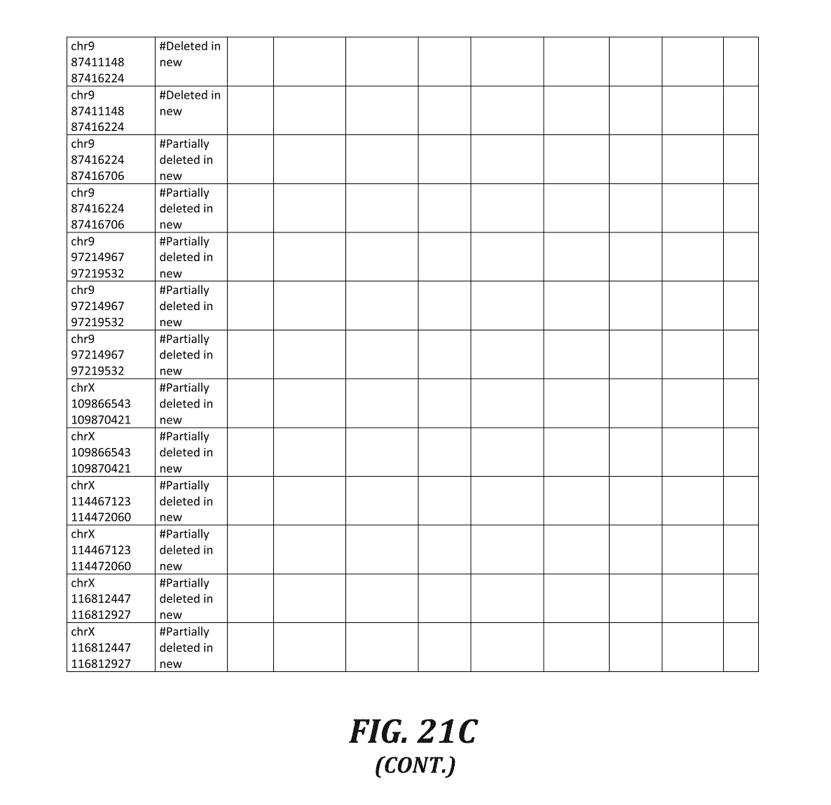

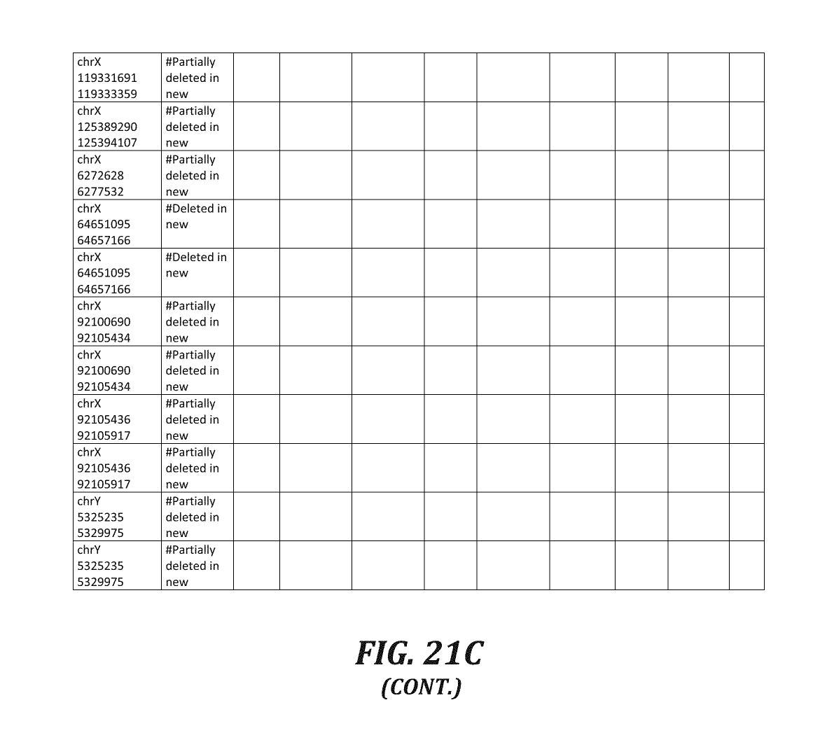

(FIGS. 1A-1D): A total of 36 statistically significant genes that are differentially expressed in human zygotes vs 8-cell human embryos are regulated by the HERVH/LBP9 in hESC. Expression of 14 of these genes is significantly different in euploid versus aneuploid human embryos (FIGS. 1A and 1C), whereas expression of 22 of these genes is not significantly different in euploid versus aneuploid human embryos (FIG. 1B). Similarly, expression signatures of 174 HERVH-regulated genes are distinct in developmentally viable and non-viable human zygotes (q<0.0005; A, FIG. 1D). Genes up-regulated in developmentally non-viable human zygotes are highlighted.

(FIGS. 1E-1H): Microarray analysis identifies gene expression signatures of HERVH-regulated genes in matured human oocytes.

FIGS. 2A-2M collectively illustrate single-cell next generation sequencing (FIGS. 2A-2J) and microarray gene expression analysis (FIGS. 2k-2M) of the individual SCARs loci (FIGS. 2A-2H), SCARs-regulatory sequences of the IncRNA HPAT3 (FIGS. 21 and 2J), and SCARs-regulated protein-coding genes (FIGS. 2k-2M) at various stages of the human preimplantation embryonic development (FIGS. 2A-2J) and in clinical samples of normal prostate epithelia, normal prostate stroma, benign prostatic hyperplasia, atrophic lesions in the prostate, putative prostate cancer precursor lesions of the prostatic intraepithelial neoplasia (PIN), morphologically normal prostate epithelia adjacent to prostate cancer lesions, localized prostate cancer, and metastatic prostate cancer (FIGS. 2k-2M).

(FIGS. 2A-2J) Single-cell next generation RNA sequencing analysis of human preimplantation embryos reveals activation of expression of selected HERVH and HERVK loci in human oocytes and zygotes. Expression patterns of individual HERV loci at the each stage of human preimplantation embryos are shown. Plotted expression values were defined either by the mean expression values normalized to the expression levels in oocytes (A) or the actual measurements in every individual cell of the corresponding stage of embryonic development (B, C).

(FIGS. 2k-2M) Microarray gene expression profiling of clinical samples representing the key stages of a hypothetical sequence of malignant progression from normal prostate epithelia to metastatic prostate tumors comprising of cells resected from normal prostate epithelia, normal prostate stroma, benign prostatic hyperplasia, atrophic lesions in the prostate, putative prostate cancer precursor lesions of the prostatic intraepithelial neoplasia (PIN), morphologically normal prostate epithelia adjacent to prostate cancer lesions, localized prostate cancer, and metastatic prostate cancer.

FIGS. 3A-3D collectively illustrate changes of gene expression and gene copy numbers of SCARs-targeted protein-coding genes manifest significant associations with the long-term survival of cancer patients. Gene copy numbers and mRNA expression levels of protein coding genes comprising structural components of the host/virus chimeric transcripts were evaluated for associations with long-term survival probabilities of cancer patients defined by the Kaplan-Meier survival analysis in TCGA Pan-cancer databases comprising 5,158 clinical samples across 12 TCGA cohorts (PANCAN12 study of 12 distinct cancer types) and 12,093 clinical samples across all TCGA cohorts. Examples of SCARs-targeted genes manifesting significant associations of gene expression changes (FIGS. 3A-3C) and gene copy number alterations (FIG. 3D) with the long-term survival of cancer patients of TCGA PANCAN12 study are shown (FIGS. 3A, 3C, and 3D). Representative examples of these associations for TCGA cohorts of three individual types of cancer [prostate cancer (n=568), breast cancer (n=1,241), and rectal cancer (n=187)] are shown in (FIG. 3B). Gene expression heatmaps and corresponding Kaplan-Meier survival curves are shown in (FIG. 3A). Heatmaps of gene expression (left images) and copy numbers (right images) and associated Kaplan-Meier survival curves are shown in (FIG. 3D). Vertical dashed lines depict the ten years survival data points. Corresponding p values are reported in the Data Set S1 (Tables 4-9).

FIGS. 4A-4D collectively illustrate protein alignments of translated amino acid sequences of the human-specific virus/host chimeric transcripts identify distinct patterns of conserved protein domains encoded by different SCARs loci. Nucleotide sequences of human-specific chimeric transcripts were translated into amino acid sequences and subjected to the BLAST protein alignment analyses as described in the Materials and Methods. Note that the most frequently represented conserved protein domains within translated amino acid sequences encoded by human-specific SCARs-derived host/virus chimeric transcripts is the GVQW (SEQ ID NO:1) amino acid sequence (FIGS. 4A, 4C, and 4D).

FIGS. 5A-5D collectively illustrate the evolutionary tracing of human-specific expansion of the GVQW conserved protein domain originated from the identical nucleic acid sequences of human-specific chimeric virus/host transcripts of SCARs on chrX:278899-284216 and chrY:278899-284216. Nucleotide sequences encoding the GVQW conserved domain were expanded to include a few adjacent amino acids, which was sufficient to obtain the SCARs' locus-specific nucleotide sequences. The genomic origin of the GVQW-encoding sequences was inferred based on the 100% nucleotide sequence identities of a given genomic sequence and the corresponding locus-specific SCARs-derived sequence. The BLAST algorithm was utilized to determine the numbers of GVQW-encoding nucleotide sequences in genomes of humans and hon-human primates, which are 100% identical to the sequences of chimeric virus/host transcripts encoded by the specific SCARs' loci. Note that no GVQW conserved protein domain-encoding sequences were detected in the mouse and rat genomes. Only GVQW-encoding sequences originated from SCARs transcripts on chrX:278899-284216 and/or chrY:278899-284216 appear markedly expanded in the human genome (red colored bar in FIG. 3C) and this expansion is associated with marked enrichment in the human proteome compared with other Great Apes of the number of proteins harboring conserved GVQW domains (FIG. 3D). Sequence reference numbers for indicated sequences are as follows: GVQW (SEQ ID NO:1), GVQWRDL (SEQ ID NO:2), QAGVQWRDL (SEQ ID NO:3), and AQAGVQWRDL (SEQ ID NO:4).

FIGS. 6A-6B illustrate changes of gene-level copy numbers of 21 zinc finger proteins harboring GVQW conserved protein domains manifest significant associations with the long-term survival of cancer patients diagnosed with 29 distinct types of malignancies. Gene copy numbers of all identified to date zinc finger proteins harboring GVQW conserved protein domains were evaluated for associations with long-term survival probabilities of cancer patients defined by the Kaplan-Meier survival analysis of TCGA Pan-cancer databases comprising 12,093 clinical samples across all TCGA cohorts representing 29 cancer types. Heatmaps of gene copy number changes (FIG. 6A) and associated Kaplan-Meier survival curves (FIG. 6B) are shown. Results of the Kaplan-Meier survival analyses are shown for 21 zinc finger proteins harboring GVQW conserved protein domains and three SCARs-targeted zin finger proteins (ZNF443; ZNF587; ZNF814). The reported p values are from the Kaplan-Meier survival curves generated by the Xena Cancer Genome Browser data visualization tools (xena.ucsc.edu).

FIGS. 7A-7D collectively illustrate the somatic non-silent mutations' signatures of the clinical intractability of malignant tumors defined by the decreased survival and increased likelihood of death from cancer.

FIG. 7A: Identification of the eighteen genes harboring somatic non-silent mutation signatures of death from cancer phenotypes. The eighteen top-scoring human genes were identified in which the largest numbers of somatic non-silent mutations (SNMs) were detected in 12,093 tumor samples across all TCGA cohorts, provided a requirement is met that the presence of these mutations in tumors is associated with significantly increased likelihood of death from cancer defined by the Kaplan-Meier survival analysis. Top panel shows distributions of SNMs of the 18 genes among patients' tumor samples aligned to the SNMs' profile of the TP53 gene. The numbers of cancer patients with SNMs of each of the 18 genes are reported as the percent of events. Shaded area highlights the relative number of cancer patients without SNMs. Note that Kaplan-Meier survival curves for each of these 18 genes identify patients with significantly decreased survival probability and increased likelihood of death from cancer. Therefore, detection of SNMs in each of these eighteen genes isolated from tumor samples is associated with poor long-term prognosis of cancer patients compared with patients whose tumors do not have SNMs of these genes (FIG. 5A). Underlined gene symbols identify genes expression of which is regulated by SCARs in the hESC. Red-colored gene symbols depict SCARs-targeted genes, whereas black-colored gene symbols identify previously reported candidate cancer driver genes.

FIG. 7B: Comparisons of the Kaplan-Meier survival analyses of 7,509 cancer patients with and without SNMs in their tumors for the TP53 gene only (FIG. 7A, top left figure below); the 18-gene SNMs' signature (FIG. 7B, top right figure below); the 26-gene SNMs' signature without TP53 (FIG. 7C, bottom left figure below); the 27-gene SNMs' signature including the TP53 gene (FIG. 7D, bottom right figure below).

FIGS. 7C and 7D: Linear regression analyses of the clinical intractability of malignant tumors in patients diagnosed with 28 (FIG. 7C) and 19 (FIG. 7D) cancer types. FIG. 7C, Cancer patients' survival data from TCGA Pan-cancer cohort of 28 cancer types were utilized to calculate the percent of death events for each cancer type; the resulting values were aligned with the percent of patients with the SNMs death from cancer signatures in the corresponding groups of cancer patients and subjected to the linear regression analysis. FIG. 7D, Age-adjusted cancer incidence and death rates (per 100,000 people) in the United States for 19 cancer types were obtained from the Center for Disease Control and Prevention (CDC) United States Cancer Statistics (USCS) report; the estimated death rates for each cancer type were calculated by multiplying the corresponding values of incidence rates and percent's of patients with the SNMs death from cancer signatures; the resulting values were aligned with the actual death rates for the corresponding cancer types and subjected to the regression analysis.

FIGS. 8A-8B illustrate that protein expression changes of the SCARs stemness networks' genes manifest statistically significant associations with decreased long-term survival and increased likelihood of death from cancer.

Protein expression changes of 38 SCARs stemness networks' genes were evaluated for associations with long-term survival probabilities of cancer patients defined by the Kaplan-Meier survival analysis in TCGA Pan-cancer database comprising 5,158 clinical samples across 12 TCGA cohorts. In total, changes in the protein expression levels of 23 SCARs-regulated genes (60.5%) manifested significant associations with the long-term survival probability of cancer patients Data Set S1; (Tables 4-9)). Heatmaps of protein expression and associated Kaplan-Meier survival curves are shown. Corresponding p values are reported in the Data Set S1 (Tables 4-9).

FIG. 9. Transcriptionally active LTR7/HERVH SCARs contribute to repair of double-stranded breaks (lightning bolt) of host DNA (blue lines) by coopting the alternative non-homologous end joining (NHEJ) DNA repair pathway. Reverse transcription of SCARs RNA (dashed black line) with partial homology regions to host DNA creates DNA molecules (solid black lines) filling the gap at the site of double-stranded breaks of host DNA. A hallmark of this mechanism of SCARs-associated repair of double-stranded DNA breaks is the evidence of deletions of ancestral DNA segments (solid red lines) at the sites of insertions of the LTR7/HERVH sequences in the human genome (see Table 3 and text for further details). This process creates human-specific integration sites of SCARs and may facilitate generation of host/virus chimeric transcripts (blue/black dashed lines). DSB, double-stranded break; NHEJ, non-homologous end joining; RT, reverse transcription; SCARs, stem cell-associated retroviruses.

FIG. 10. Flow chart of a decision-making process in clinical management of cancer patients on the basis of continuing sequential sampling for monitoring of the SCAR's networks activity status in blood, serum, and plasma samples; circulating tumor cells; primary and metastatic tumor samples.

Identification of genetic and/or molecular evidence of the activated SCAR's networks at any stage of this sequence would favor the diagnosis of therapy-resistant clinically-lethal disease phenotype and trigger the requirement for the immediate consideration of the following therapy selection choices: the “next-in-line” aggressive treatment protocols; novel therapies specifically targeting SCAR's pathways and/or therapeutic interventions considered suitable for patients with malignant tumors manifesting the active status of SCAR's networks. CTC, circulating tumor cell; FFPE, formalin-fixed paraffin embedded. Adopted from: Glinsky, GV. 2008. “Stemness” genomics law governs clinical behavior of human cancer: Implications for decision making in disease management. Journal of Clinical Oncology, 26: 2846-53.

FIGS. 11A-11K (related to FIGS. 4A-4D) provide additional examples of distinct and common patterns of the conserved protein domain expression within translated amino acid sequences of the host/virus chimeric transcripts encoded by endogenous human SCARs in the hESC. Nucleotide sequences of human-specific chimeric transcripts were translated into amino acid sequences and subjected to the protein alignment analyses using the protein BLAST algorithm (blast.ncbi.nlm.nih.gov) and associated web-based tools for identification and visualization of conserved protein domains (ncbi.nlm.nih.gov/Structure), which were described in details elsewhere [80, 81].

Protein alignments of translated amino acid sequences of the human-specific virus/host chimeric transcripts identify distinct patterns of conserved protein domains encoded by different SCARs loci. Nucleotide sequences of human-specific chimeric transcripts were translated into amino acid sequences and subjected to the BLAST protein alignment analyses as described in the Materials and Methods. Note that the most frequently represented conserved protein domains within translated amino acid sequences encoded by human-specific SCARs-derived host/virus chimeric transcripts is the GVQW amino acid sequence (SEQ ID NO:1). Sequence reference numbers for additional sequences as follows: GVQWRDL (SEQ ID NO:2), QAGVQWRDL (SEQ ID NO:3), and AQAGVQWRDL (SEQ ID NO:4).

FIGS. 12A-12D (related to FIGS. 6A and 6B) illustrate that changes of gene expression and gene copy numbers of zinc finger proteins harboring GVQW conserved protein domains manifest significant associations with the long-term survival of cancer patients. Gene copy numbers (FIG. 12D) and mRNA expression levels (FIGS. 12A-12C) of zinc finger proteins harboring GVQW conserved protein domains were evaluated for associations with long-term survival probabilities of cancer patients defined by the Kaplan-Meier survival analysis of cancer patients diagnosed with prostate cancer (n=568); breast cancer (n=1,241); colon cancer (n=550); rectal cancer (n=187); pancreatic cancer (n=196); and TCGA Pan-cancer databases comprising 5,158 clinical samples across 12 TCGA cohorts (PANCAN12 study of 12 distinct cancer types). Representative examples of zinc finger proteins with GVQW conserved protein domains that manifest significant associations of gene expression changes (FIGS. 12A-12C) in TCGA cohorts of five individual types of cancer [prostate cancer (FIG. 12A); breast cancer (FIG. 12B; FIG. 12C, bottom left panel); colon cancer (FIG. 12C; top left panel); rectal cancer (FIG. 12C; top right panel); and pancreatic cancer (FIG. 12C, bottom right panel)] are shown. Examples of zinc finger proteins with GVQW conserved protein domains manifesting significant associations of gene copy number alterations with the long-term survival of cancer patients of TCGA PANCAN12 study are shown in FIG. 4D. Gene expression heatmaps and corresponding Kaplan-Meier survival curves are shown in (FIGS. 12A-12C). Heatmaps of gene expression (left images) and exon expression (right images) and associated Kaplan-Meier survival curves are shown in (FIG. 12C). Heatmaps of gene expression (left images) and copy numbers (right images) and associated Kaplan-Meier survival curves are shown in (FIG. 12D). Corresponding p values are reported in the Data Set S1 (Tables 4-9).

FIGS. 13A and 13B (related to FIGS. 7A-7D) illustrate additional Kaplan-Meier survival analyses of the classification performance of SNMs genes including only patients with the complete clinical records of the follow-up survival data.

FIG. 13A: Comparisons of the Kaplan-Meier survival analyses of 7,258 cancer patients with and without SNMs in their tumors (top and bottom left figures) and cancer patients stratified into sub-groups of identical size (n=2,419) after sorting in the ascending order of their survival time (top and bottom left figures). In this analysis only patients with the complete clinical records of the follow-up survival data were included.

FIG. 13B: Visualization of mutations' fingerprints of genes harboring the SNMs signatures of death from cancer phenotypes. Note that these genes isolated from clinical tumor samples appear “littered” with mutations, a vast majority of which is represented by the SNMs.

FIGS. 14A-14D illustrate changes of gene-level copy numbers of master transcriptional regulators of SCARs-associated stemness networks in the hESC (boxed Kaplan-Meier plots of the KLF4; LBP9; NANOG; and POU5F1 genes) and the SNMs' death from cancer signatures' genes manifest statistically significant associations with decreased long-term survival and increased likelihood of death from cancer. Gene-level copy number changes of indicated protein coding genes were independently evaluated for associations with long-term survival probabilities of cancer patients defined by the Kaplan-Meier survival analysis in two TCGA Pan-cancer databases comprising 5,158 clinical samples across 12 TCGA cohorts (FIGS. 14A and 14C) and 12,093 clinical samples across 29 TCGA cohorts (FIGS. 14B and 14D). Note, that strikingly similar results were observed for the copy number changes of the BMI1 (bottom left panels in FIGS. 14C and 14D) and EZH2 (bottom right panels in FIGS. 14C and 14D) genes, associations of which with the activation of the Polycomb chromatin silencing pathway and stemness gene expression signatures in tumors from cancer patients with increased likelihood of death from cancer were previously documented (37-51). Corresponding p values are reported in the Data Set S1 (Tables 4-9).

FIG. 15 illustrates Kaplan-Meier survival analyses of therapy outcomes in prostate cancer patients stratified into distinct sub-groups based on expression profiles of the 11-gene death from cancer signature and expression signatures of three SCARs network genes (PLCXD1, HKR1, ZNF283).

FIG. 16 is a table disclosing a panel of 42 genes for the analysis of the somatic non-silent mutations which were identified based on significant associations with the increased likelihood of therapy failure and death from cancer in multiple pan-cancer databases.

FIGS. 17A-17C are tables that disclose the following:
FIG. 17A: Two-tailed p value: 0.00090474; p=0.0009; related to FIG. 7C.
FIG. 17B: 2 -tailed p value; related to FIG. 7D.
FIG. 17C: Related to FIGS. 7A-7D.

FIGS. 18A and 18B are tables that disclose the following:
FIG. 18A: ChrY_ChrX
FIG. 18B: chr3_chr11

FIGS. 19A and 19B are tables that disclose the following:
FIG. 19A: 74 genes.
FIG. 19B: 55 genes.

FIGS. 20A-20C are tables that disclose the following:
FIG. 20A: HERVH-loci manifesting the most significant activation at the zygote stage of human embryogenesis. Related to FIGS. 2A-2M.
FIG. 20B HERVK-; HERVH-; and other SCARs loci manifesting the most significant activation at the zygote stage of human embryogenesis. Related to FIGS. 2A-2M.
FIG. 20C: SCARs sequences implicated in the human embryogenesis and development of pathological conditions in human subjects.
FIGS. 21A-21C are tables that disclose the following:
FIG. 21A: 64 HERV1 human-specific chimeric transcripts (Bonobo & Chimp alignments failures).
FIG. 21B is a table.
FIG. 21C is a table.

### DETAILED DESCRIPTION

A wide variety of cancer treatment protocols have been developed in recent years, including novel methods of personalized, target-tailored cancer therapies. Often, very aggressive cancer therapy is reserved for late stage cancers due to unwanted side effects produced by such therapy. However, even such aggressive therapy commonly fails at such a late stage. The ability to identify cancers responsive only to the most aggressive therapies at an earlier stage could greatly improve the prognosis for patients having such cancers.

In recent years, potentially useful markers predictive of such outcomes have been identified. Glinsky, G.V. et al., J. Clin. Invest. 113: 913-923 (2004) teaches that gene expression profiling predicts clinical outcomes of prostate cancer. Van ′t Veer et al., Nature 415: 530-536 (2002) teaches that gene expression profiling predicts clinical outcomes of breast cancer. Glinsky et al., J. Clin. Invest. 115: 1503-1521 (2005) teaches that altered expression of the BMI1 oncogene is functionally linked with the self-renewal state of normal and leukemic stem cells as well as a poor prognosis profile of an 11-gene death-from-cancer signature predicting therapy failure in patients with multiple types of cancer. These studies utilized the microarray gene expression analysis approach.

There is, therefore, a continuous and ever-growing need for highly accurate methods for early diagnosis of cancer and for prognostic assays for cancer therapy that are readily adaptable to the clinical setting. Such methods should utilize state of the art technologies that can be readily carried out in clinical laboratories, and should accurately predict the likelihood of resistance of various cancers to be applied to standard therapeutic regimens.

A very large number of attempts have been made to discover, define, and design treatments, develop treatments, and to treat metastatic and intractable cancers, principally by either attacking basic mechanisms of rapid cell growth or aberrant cancer cell metabolic pathways, with little success. Recently, some methods of enabling or re-enabling the immune system in its attack on tumors and micro-metastases has shown much more promising data in trials and commercial use, but the majority of patients with metastatic and intractable disease have proven refractory to even these immune-modulating therapies. There is, therefore, a need for new cancer therapies which, either used as sole therapeutic agents or in combination with other modalities—particularly immune-modulation—are designed to fundamentally attack the cellular mechanisms allowing the metastatic phenotype. Such new therapies should be derived from an understanding of the critical gene signatures responsible for metastasis and survival of cancer cells.

Somatic mutations and chromosome instability are hallmarks of genomic aberrations in cancer cells. Aneuploidies represent common manifestations of chromosome instability, which is frequently observed in human embryos and malignant solid tumors. Activation of human endogenous retroviruses (HERV)-derived loci is documented in preimplantation human embryos, hESC, and multiple types of human malignancies. It remains unknown whether the HERV activation may highlight a common molecular pathway contributing to the frequent occurrence of chromosome instability in the early stages of human embryonic development and the emergence of genomic aberrations in cancer.

Single cell RNA sequencing analysis of human preimplantation embryos reveals activation of specific LTR7/HERVH loci during the transition from the oocytes to zygotes and identifies HERVH network signatures associated with the aneuploidy in human embryos. The correlation pattern's analysis links transcriptome signatures of the HERVH network activation of the in vivo matured human oocytes with gene expression profiles of clinical samples of prostate tumors supporting the existence of a cancer progression pathway from putative precursor lesions (prostatic intraepithelial neoplasia) to localized and metastatic prostate cancers. Tracking signatures of HERVH networks' activation in tumor samples from cancer patients with known long-term therapy outcomes enabled patients' stratification into sub-groups with markedly distinct likelihoods of therapy failure and death from cancer.

Genome-wide analyses of human-specific genetic elements of stem cell-associated retroviruses (SCARs)-regulated networks in 12,093 clinical tumor samples across 29 cancer types revealed pan-cancer genomic signatures of clinically-lethal therapy resistant disease defined by the presence of somatic non-silent mutations (SNMs), gene-level copy number changes, transcripts' and proteins' expression of SCARs-regulated host genes. More than 73% of all cancer deaths occurred in patients whose tumors harbor the SNMs' signatures. Linear regression analysis of cancer intractability in the United States population demonstrated that organ-specific cancer death rates are directly correlated with the percentages of patients whose tumors harbor the SNMs' signatures.

SCARs-encoded RNA molecules possess intrinsic protein-coding potentials including amino acid sequences defined as conserved protein domains (CPD). Mapping of SCARs-encoded CPDs revealed thousands of locus-specific fingerprints of CPDs scattered genome-wide. The evolutionary expansion of SCARs' sequences encoding specific CPDs resulted in a marked enrichment in the human proteome of the unique protein sequences on which the CPD is found. These results indicate that diseased cells with high expression levels of SCARs RNA are likely to carry a markedly increased load of SCARs RNA-encoded peptides providing attractive and highly specific molecular targets for immunotherapeutic interventions.

A systematic analysis of molecular structures of human-specific virus/host chimeric transcripts demonstrates that a hallmark feature of SCARs' integration in the human genome is a multispecies deletion pattern of ancestral DNA. The cross-species tracing of SCARs' loci with human-specific insertions and deletions suggests a potential role in the repair of double-stranded DNA breaks, highlighting a putative biological function of SCARs that may enhance the immediate survival and fitness of host cells. On the evolutionary scale, in addition to seeding thousands of human-specific regulatory sequences, the SCARs' activity appears involved in DNA repair and spreading sequences of specific CPDs throughout the human genome.

Examples presented herein demonstrate that awakening of SCARs-regulated stemness networks in differentiated cells is associated with development of a diverse spectrum of genomic aberrations subsequently readily detectable in multiple types of clinically lethal malignant tumors and likely contributing to emergence of therapy-resistant phenotypes.

Key words: human endogenous stem cell-associated retroviruses (SCARs); human-specific regulatory sequences; human ESC; human embryos; pluripotent state regulators; NANOG; POU5F1 (OCT4); CTCF; LTR7 RNAs; long terminal repeats, LTR; LTR7/HERVH; LTR5HS/HERVK; therapy-resistant cancers; cancer stem cells

### LIST OF ABBREVIATIONS

HERV, human endogenous retroviruses

hESC, human embryonic stem cells

LINE, long interspersed nuclear element

IncRNA, long non-coding RNA

lincRNA, long intergenic non-coding RNA

LTR, long terminal repeat

NANOG, Nanog homeobox

POU5F1, POU class 5 homeobox 1

SCARs, stem cell associated retroviruses

TOGA, The Cancer Genome Atlas

TE, transposable elements

TF, transcription factor

TFBS, transcription factor-binding sites

sncRNA, small non coding RNA

### STEM CELL-ASSOCIATED RETROVIRUSES (SCARS)

Activity of endogenous retroviruses is suppressed in human cells to restrict the potentially harmful effects of mutations on functional genome integrity and to ensure the maintenance of genomic stability. Human embryonic stem cells (hESCs) and early-stage human embryos seem markedly different in this regard. Expression of human endogenous retroviruses (HERV), in particular, HERVH and HERVK subfamilies, is markedly activated in hESCs [1-3]. An enhanced rate of insertion of LTR7/HERVH sequences in the human genome appears to be associated with binding sites for pluripotency core transcription factors [1; 3; 4], including human-specific transcription binding sites [3], and long noncoding RNAs [5]. Analysis of transcription factor binding sites in hESC suggests that expression of HERVH is regulated by the pluripotency regulatory circuitry, since 80% of long terminal repeats (LTRs) of the 50 most highly expressed HERVH loci are occupied by pluripotency core transcription factors, including NANOG and POU5F1 [1]. Furthermore, transposable elements (TE) -derived sequences, most notably LTR7/HERVH, LTR5_Hs/HERVK, and L1HS, harbor 99.8% of the candidate human-specific regulatory sequences (HSRS) with putative transcription factor-binding sites (TFBS) in the genome of hESC [3]. Based on the common functional features of these specific families of HERVs, which are mediated by their active expression in the human embryos and hESC [6-9], they were designated as the endogenous human stem cell-associated retroviruses (SCARs).

Recent studies highlighted mechanisms of activation and putative biological functions of SCARs in human preimplantation embryos and embryonic stem cells. The LTR7/HERVH subfamily is rapidly demethylated and upregulated in the blastocyst of human embryos and remains highly expressed in hESC [10]. Sequences of LTR7, LTR7B, and LTR7Y, which typically harbor the promoters for the downstream full-length HERVH-int elements, were found expressed at the highest levels and were the most statistically significantly up-regulated retrotransposons in human ESC and induced pluripotent stem cells, iPSC [11]. It has been demonstrated that LTRs of HERVH subfamily, in particular, LTR7, function in hESC as enhancers and HERVH sequences encode nuclear non-coding RNAs, which are required for maintenance of pluripotency and identity of hESC [12]. Transient spatiotemporally controlled hyper-activation of HERVH is required for reprogramming of differentiated human cells toward induced pluripotent stem cells (iPSC), maintenance of pluripotency and reestablishment of differentiation potential [13]. Failure to control and silence the LTR7/HERVH activity leads to the differentiation-defective phenotype in neural lineage [13, 14]. Activation of L1 retrotransposons may also contribute to these processes because significant activities of both L1 transcription and transposition were recently reported in iPSC of humans and other great apes [15]. Single-cell RNA sequencing of human preimplantation embryos and embryonic stem cells [16, 17] enabled identification of specific distinct populations of early human embryonic stem cells defined by marked activation of specific retroviral elements [18].

Discovery of endogenous human SCARs and compelling evidence of their essential role in human embryogenesis may have some immediate practical implications. Heterogeneous populations of human ESCs and iPSC contain naive-state stem cells that have the most broad and robust multi-lineage developmental potentials and, therefore, hold great promise for a multitude of life-saving therapeutic applications in regenerative medicine. Consistent with definition of increased LTR7/HERVH expression as a hallmark of naive-like hESCs, a sub-population of hESCs and human induced pluripotent stem cells (hiPSCs) with markedly elevated LTR7/HERVH expression manifests key properties of naive-like pluripotent stem cells [19]. Furthermore, human naive-like pluripotent stem cells can be genetically tagged, successfully isolated and maintained in vitro based on markers of elevated transcription of LTR7/HERVH [19]. Embryonic stem cell-specific transcription factors NANOG, POU5F1, KLF4, and LBP9 drive LTR7/HERVH transcription in human pluripotent stem cells [19]. Targeted interference with HERVH activity and HERVH-derived transcripts severely compromises self-renewal functions of human pluripotent stem cells [19].

Similar to the LTR7/HERVH subfamily, transactivation of LTR5_Hs/HERVK by pluripotency master transcription factor POU5F1 (OCT4) at hypomethylated LTRs, which represent the most evolutionary recent genomic integration sites of HERVK retroviruses, induces HERVK expression during normal human embryogenesis [20]. It coincides with embryonic genome activation at the eight-cell stage, continuing through the stage of epiblast cells in preimplantation blastocysts, and ceasing during hESC derivation from blastocyst outgrowths [20]. The unequivocal experimental evidence of HERVK activation during human embryogenesis has been reported by Grow et al. [20]. They demonstrated the presence of HERVK viral-like particles and Gag proteins in human blastocysts, supporting the idea that endogenous human retroviruses are active and functional during early human embryonic development. Consistent with this hypothesis, overexpression of HERVK virus-accessory protein Rec in pluripotent cells was sufficient to increase the host protein IFITM1 level and inhibit viral infection [20], suggesting that this anti-viral defense mechanism in human early-stage embryos may be triggered by HERVK activation. Detailed analysis of how activation of retrotransposons orchestrates species-specific gene expression in embryonic stem cells is presented in the recent review [21], highlighting the fine regulatory balance established during evolution between activation and repression of specific retrotransposons in human cells.

Recent experiments identified key effector molecules mediating critical biological activities of SCARs in hESC. SCARs-derived long noncoding RNAs have been described as the essential regulatory molecules for maintaining pluripotency, functional identity, and integrity of hESC [12]. Collectively, these experiments conclusively established the essential role of the sustained yet tightly spatiotemporally controlled activity of specific endogenous retroviruses for pluripotency maintenance and functional identity of human pluripotent stem cells, including hESC and iPSC. It has been hypothesized that awakening of SCARs may be associated with activation of stemness genomic networks in cancer cells and the emergence of clinically-lethal death from cancer phenotypes in patients diagnosed with multiple types of malignant tumors [6-9].

In summary, the emerging consensus view is that spatiotemporally controlled activation of endogenous stem cell-associated retroviruses (SCARs) in human preimplantation embryos, specifically LTR7/HERVH and LTR5_Hs/HERVK subfamilies, is required for the pluripotency maintenance, functional identity and integrity of the naive-state ESC, and anti-viral resistance of the early-stage human embryos. Expression of SCARs is epigenetically silenced in differentiated human cells and failure to control and efficiently silence the SCARs activity leads to differentiation-defective phenotypes. Reversal of epigenetic silencing of SCARs loci in cancer cells appears associated with activation of SCARs expression in multiple types of human tumors (reviewed in 9 and references therein).

In this contribution, single cell RNA sequencing analysis of human preimplantation embryos reveals activation of specific LTR7/HERVH loci during the transition from the oocytes to zygotes and identifies HERVH network signatures associated with aneuploidy in human embryos. The correlation patterns' analysis links transcriptome signatures of the HERVH network activation of the in vivo matured human oocytes with gene expression profiles of clinical samples of prostate tumors supporting the existence of a cancer progression pathway from prostatic intraepithelial neoplasia to localized and metastatic prostate cancers. Manifestation of a diverse spectrum of genomic aberrations in malignant tumors from cancer patients with clinically lethal disease has been associated with the activation of SCARs networks in cancer cells. The Cancer Genome Atlas (TCGA)-guided analyses of SCARs networks in 12,093 clinical samples across all TCGA cohorts representing 29 cancer types revealed pan-cancer genomic signatures of clinically-lethal therapy resistant disease defined by the gene expression, gene-level copy number changes, protein expression, somatic non-silent mutations of SCARs-associated protein-coding genes and non-coding RNA loci.

### DESCRIPTION OF EXPERIMENTAL EXAMPLES

Single-cell transcriptome analysis reveals active transcription from selected LTR7/HERVH loci and altered expression of LTR7/HERVH-regulated genes in aneuploidy-prone and developmentally non-viable human zygotes

Chromosome instability is common in the early-stage human embryonic development and aneuploidies observed in 50-80% of cleavage-stage human embryos [Vanneste E, Voet T, Le Caignec C, Ampe M, Konings P, Melotte C, Debrock S, Amyere M, Vikkula M, Schuit F, Fryns J P, Verbeke G, D'Hooghe T, Moreau Y, Vermeesch J R. Chromosome instability is common in human cleavage-stage embryos. Nat Med. 2009; 15 :577-83; Johnson D S, Gemelos G, Baner J, Ryan A, Cinnioglu C, Banjevic M, Ross R, Alper M, Barrett B, Frederick J, Potter D, Behr B, Rabinowitz M. Preclinical validation of a microarray method for full molecular karyotyping of blastomeres in a 24-h protocol. Hum Reprod. 2010; 25: 1066-75; Chavez S L, Loewke K E, Han J, Moussavi F, Coils P, Munne S, Behr B, Reijo Pera R A. Dynamic blastomere behaviour reflects human embryo ploidy by the four-cell stage. Nat Commun. 2012; 3:1251; Vera-Rodriguez M, Chavez S L, Rubio C, Reijo Pera R A, Simon C. Prediction model for aneuploidy in early human embryo development revealed by single-cell analysis. Nat Commun. 2015; 6: 7601; Yanez L Z, Han J, Behr B B, Pera R A, Camarillo D B. Human oocyte developmental potential is predicted by mechanical properties within hours after fertilization. Nat Commun. 2016; 7: 10809].

Aneuploidies in human embryos impair proper development leading to the cell cycle arrest, loss of cell viability, and developmental failures. Single-cell transcriptome analyses demonstrated that gene expression signatures of zygotes could reliably predict the development of euploid and aneuploid human embryos as well as distinguish between developmentally viable and non-viable zygotes [Vera-Rodriguez M, Chavez S L, Rubio C, Reijo Pera R A, Simon C. Prediction model for aneuploidy in early human embryo development revealed by single-cell analysis. Nat Commun. 2015; 6: 7601; Yanez L Z, Han J, Behr B B, Pera R A, Camarillo D B. Human oocyte developmental potential is predicted by mechanical properties within hours after fertilization. Nat Commun. 2016; 7: 10809].

The validity test of the hypothesis that activation of specific LTR7/HERVH loci is associated with development of aneuploidies in human embryos must conform to these experimental paradigms and comply with the following postulates:
Increased LTR7/HERVH expression should be readily detectable in human zygotes;
Cells with activated LTR7/HERVH loci at the zygote stage should not persist during the subsequent stages of human embryogenesis; and
Gene expression signatures of aneuploidy-prone human embryos should harbor the significant number of LTR7/HERVH-regulated genes.

Analysis of human embryonic development-associated genes demonstrates that the number of LTR7/HERVH-regulated genes is significantly enriched among genes that are differentially expressed in aneuploid compared with euploid embryos (Table 1A). In contrast, no significant enrichment of the LTR7/HERVH-regulated genes was documented in other gene sets representing six distinct gene expression categories of human embryonic development-associated genes (Table 1A). Consistent with the hypothesis that activation of LTR7/HERVH loci is associated with development of aneuploidies in human embryos, the significant correlation was observed between the gene expression signature of shHERVH-treated hESC and the gene expression profile of zygotes versus 8-cell embryos comprising of genes that are differentially expressed in aneuploid versus euploid embryos (FIGS. 1A-1K). In contrast, no significant correlation was documented between the expression signature of shHERVH-treated hESC and the gene expression profile of zygotes versus 8-cell stage embryos comprising of genes that are not differentially expressed between aneuploidy versus euploid embryos (FIGS. 1A-1K). Consistent with the idea that the expression of HERVH-regulated genes distinguishes human zygotes with distinct developmental potentials, it has been observed that fifty percent of all genes differentially expressed in developmentally viable versus non-viable zygotes comprised of genes regulated by the LBP9/HERVH in hESC (FIGS. 1A-1K).

Next, the validity of a prediction was tested that activation of LTR7/HERVH expression occurs early in the embryogenesis following the fertilization of oocytes and, therefore, it could be readily observed in human zygotes during the single cell transcriptome analysis of human preimplantation embryos. In agreement with this idea, the significant activation of several defined LT7/HERVH loci was observed during transition of the fertilized human oocytes to zygotes (FIGS. 2A-2M). Notably, the increased LTR7/HERVH expression in zygotes was restricted to only limited number of specific LTR7/HERVH loci and failed to persist beyond the 8-cell stage (FIGS. 2A-2M). As expected, most of the LTR7/HERVH loci remain silent during the early-stage embryogenesis and undergo massive activation during the late blastocyst stage, the epiblast formation, and at the onset of hESC creation [1-14; 16-21]. In agreement with the hypothesis, a vast majority of cells with activated LTR7/HERVH loci in zygotes did not persist during the subsequent stages of human embryogenesis (FIGS. 2A-2M), with the exception of the pattern 4 cells manifesting markedly increased LTR7/HERVH expression at the epiblast and hESC creation stages of embryogenesis. Activation of the LTR7/HERVH loci manifesting the pattern 4 of expression profiles during human embryogenesis is likely related to the creation of the ground-state pluripotency state and naive hESC. This hypothesis is further corroborated by the single-cell transcriptome analyses of expression profiles of the LTR7/HERVH sequences of HPAT3 lincRNA which plays an important role in pluripotency regulation and maintenance networks of hESC (FIGS. 2A-2M).

Gene expression signature of the LTR7/HERVH network activation in human oocytes distinguishes prostate cancer precursor lesions, localized and metastatic prostate cancers from normal prostate epithelia and benign prostatic hyperplasia.

During embryogenesis no transcription occurs before the embryonic genome activations, indicating that the early stages of embryogenesis are controlled exclusively by the maternal genetic information inherited exclusively from the oocytes. The major wave of transcriptional activation of embryonic genome was observed at the four-to eight-cell stage of human embryogenesis [Dobson A T, Raja R, Abeyta M J, Taylor T, Shen S, Haqq C, Pera R A. The unique transcriptome through day 3 of human preimplantation development. Hum. Mol. Genet. 2004; 13: 1461-1470]. These considerations suggest that the increased expression of the HERVH loci observed in human zygotes may be related to their active transcriptional status in oocytes. Consistent with this idea, analysis of the transcriptome of human metaphase II oocytes obtained within minutes after their removal from the ovary [Kocabas A M, Crosby J, Ross P J, Otu H H, Beyhan Z, Can H, Tam W L, Rosa G J, Halgren R G, Lim B, Fernandez E, Cibelli J B. The transcriptome of human oocytes. Proc Natl Acad Sci U S A. 2006; 103: 14027-32] identified a large set of differentially-expressed HERVH-regulated genes (FIGS. 1A-1K). Furthermore, single cell transcriptome analysis of human preimplantation embryos revealed direct experimental evidence of the expression of selected LTR7/HERVH loci in human oocytes [FIGS. 2A-2M]. Identification of the gene expression signature of LTR7/HERVH network activation in human oocytes provides the opportunity to determine whether this gene signature may be useful for detection of the LTR7/HERVH transcriptome activation in clinical samples of malignant tumors. Remarkably, this analysis reveals that the gene expression signature of the LTR7/HERVH network activation in human oocytes appears to distinguish prostate cancer precursor lesions, localized and metastatic prostate cancers from clinical samples of normal prostate epithelia, stroma, and benign prostatic hyperplasia (FIGS. 3A-3D).

These observations strongly indicate that activation of the LTR7/HERVH transcriptome occurs in large sub-sets of clinical samples of prostatic intraepithelial neoplasia constituting prostate cancer precursor lesions (31-46% of samples), localized prostate adenocarcinomas (22-28% of samples), and metastatic prostate cancers (45-60% of samples). Collectively, these results argue that activation of the LTR7/HERVH regulatory network occurs early during development of clinically significant prostate cancer and manifests the persistence during prostate cancer progression from putative precursor lesions (prostatic intraepithelial neoplasia) to localized and metastatic prostate cancers.

Differential expression of human-specific chimeric host/virus transcripts segregates cancer patients into subgroups with markedly distinct long-term survival probabilities

It has been hypothesized that awakening of SCARs is associated with activation of stemness genomic networks in cancer cells and the emergence of clinically-lethal death from cancer phenotypes in patients diagnosed with multiple types of malignant tumors [6-9]. Insertions of SCARs in defined regions of the hESC genome appear to markedly affect the expression of host genes and chimeric host/virus transcripts by creating alternative promoters, exonization, and alternative splicing (18-20). These data suggest that genomic signatures of the activation of SCARs networks may consist of different classes of genetic elements, including SCARs-derived transcripts, SCARs-regulated protein-coding genes, chimeric host/virus transcripts, and non-coding RNAs. Interestingly, while ˜75% of the full-length LTR7/HERVH loci appear highly conserved in humans and non-human primates (Table 1), more than 300 loci represent candidate human-specific regulatory elements, thus underscoring the need for exploration of biological roles of both conserved primate-specific and unique to human regulatory SCARs-derived sequences. Of note, full-length human-specific LTR7/HERVH sequences are significantly enriched among the transcriptionally active loci compared with the inactive LTR7/HERVH loci (Table 1). Therefore, mRNA expression profiles of protein-coding genes comprising structural components of the host/virus chimeric transcripts may be useful for the assessment of the potential clinical relevance of the locus-specific SCARs activation in human tumors.

To assess the potential clinical relevance of SCARs activation, the patterns of changes of mRNA expression levels of protein coding genes comprising structural components of the host/virus chimeric transcripts in association with long-term survival probabilities of cancer patients defined by the Kaplan-Meier survival analysis were evaluated (FIGS. 1A-1H). The primary focus of this analysis was on the host/virus chimeric transcripts which harbor human-specific SCARs insertions and, therefore, were defined as candidate human-specific regulatory sequences (Tables 1-3).

Interrogation of two TCGA Pan-Cancer databases, comprising 5,158 clinical samples across 12 TCGA cohorts (PANCAN12 study of 12 distinct cancer types) and 12,093 clinical samples across all TCGA cohorts (genomecancersoe.ucsc.edu/proysite/xena/datapages/), demonstrates that changes of gene expression and gene copy numbers of SCARs-targeted protein-coding genes manifest two distinct association patterns with the long-term survival of cancer patients (FIGS. 1A-1H).

One of the association patterns is defined by the observations that increased gene expression levels of the SCARs-targeted genes appear associated with decreased likelihood of cancer patients' survival. This pattern was observed for the PLCXD1 and CCL26 genes (FIGS. 1A-1H). In contrast, the second association pattern is illustrated by the evidence that decreased gene expression levels of the SCARs-targeted genes are associated with decreased probabilities of cancer patients' survival. This pattern was observed for the ZNF443, LRBA, TPT1, ABHD12B, and LIN7A mRNAs (FIGS. 1A-1H).

Association patterns similar to TCGA Pan-Cancer datasets were observed during the analyses of the cancer type-specific patients' survival profiles (FIG. 1B), including TCGA Breast Cancer cohort (1,241 clinical samples); TCGA Prostate Cancer cohort (568 clinical samples); and TCGA Rectal Cancer cohort (187 clinical samples). Notably, among patients diagnosed with prostate and rectal cancers, it appears possible to identify the good prognosis sub-group of patients comprising of individuals with ˜100% survival probability more than 10 years after diagnosis and therapy (FIGS. 1A-1H and FIGS. 12A-12E). Therefore, changes of mRNA expression levels and gene copy numbers of SCARs-targeted protein-coding genes with human-specific retroviral insertions comprising structural elements of host/virus chimeric transcripts seem consistent with the hypothesis that different SCAR's activation patterns observed in malignant tumors are associated with clinically distinct outcomes in cancer patients.

Somatic non-silent mutations' fingerprints associated with increased likelihood of death from cancer For efficient evidence-based, individualized management of cancer patients and development of novel diagnostic, prognostic, and therapeutic applications, it would be particularly useful to identify the genetic signatures of somatic non-silent mutations of clinical intractability of malignant tumors, which is defined by the increased probabilities of therapy failure, disease recurrence, metastatic progression, and ultimately death from cancer. To this end, the SCARS' genomic networks and cancer drivers genes were systematically searched for genes that acquired somatic non-silent mutations, detection of which in tumor samples is associated with increased likelihood of death from cancer. Multiple statistically significant instances of this type of associations were observed: that is, genes of the SCARs-associated genomic networks acquired somatic non-silent mutations (SNMs) in malignant tumors and cancer patients having tumors with these mutations manifested a significantly decreased long-term survival probability and increased likelihood of death from cancer FIGS. 5A-5D. These observations implied that there are genes within SCARs-associated genomic networks that may function as genetic drivers of clinically lethal death from cancer phenotypes. Conversely, it was reasonable to expect that some of genes previously defined as cancer drivers may constitute a category of candidate SCARs-regulated genes.

This hypothesis has been tested by determining how many previously reported candidate cancer driver genes were also identified in independent experiments as candidate SCARs-regulated genes, which were recently discovered using shRNA approaches [19]. A total of 183 of 291 genes (63%) reported as the high-confidence cancer driver genes [22] were identified as the candidates HERVH/LBP9-regulated genes in the hESC. Similarly, 75 of 127 genes (59%) previously identified as significantly mutated genes in human tumors [23] were reported among the candidates HERVH/LBP9-regulated genes. Lastly, 325 of 572 genes (57%) of the latest release of the Cancer Gene Census (http://cancer.sanger.ac.uk/census) were identified as the candidates HERVH/LBP9-regualted genes in the hESC. Collectively, these observations indicate that a majority of genes that exhibit signals of positive selection across multiple cohorts of tumor samples and were defined as candidate cancer driver genes appears regulated by the HERVH/LBP9 stemness pathway in the hESC.

Based on these consideration, the 18-gene death from cancer SNMs' signature has been identified that segregates patients with decreased survival probability and increased likelihood of death from cancer FIGS. 5A-5D. Detection of somatic non-silent mutations in each of these eighteen genes isolated from tumor samples appears associated with poor long-term prognosis of cancer patients compared with patients whose tumors do not have somatic non-silent mutations of these genes FIGS. 5A-5D. Significantly, it has been observed that ˜70% of all cancer death events occurred in the poor prognosis patients' sub-group defined by the 18-gene death from cancer mutations' signature, whereas TP53 mutations signature alone captured less than 50% of death events FIGS. 5A-5D. The eighteen genes comprising the death from cancer SNMs' signature represent human genes in which the presence of somatic non-silent mutations were detected in a single pan-cancer dataset of 7,509 tumor samples across all TCGA cohorts and confirmed during the follow-up analyses of 9 pan-cancer datasets ranging from 1,934 to 8,272 tumor samples, provided that a requirement is met that the presence of these mutations in tumors is associated with significantly increased likelihood of death from cancer defined by the Kaplan-Meier survival analysis (see below). Notably, when the additional nine significant SNMs genes were included in the Kaplan-Meier survival analyses, the classification power of the SNM signature appears to increase only marginally FIGS. 5A-5D.

Cancer survival likelihood classification performance of the SNMs genes was confirmed using several additional analyses (FIGS. 13A and 13B). In these analyses only patients with the complete clinical records of the follow-up survival data were included. Comparisons of the Kaplan-Meier survival analyses of 7,258 cancer patients with and without SNMs in their tumors demonstrate that cancer patients whose tumors harbor at least three SNMs genes manifested the shortest median survival (1,438 days), compared with patients with two SNMs genes (median survival 1,725 days) or patients with just one SNMs gene (median survival 1,944 days). Cancer patients without SNMs genes in their tumors had the longest median survival time (4,068 days). When 7,258 cancer patients were stratified into three sub-groups of identical size (n=2,419) after sorting in the ascending order of their survival time, 63.4% of patients with the median survival of 360 days had the SNMs genes in their tumors, whereas 58.5% and 51.8% of cancer patients with the median survival of 869 days and 4,222 days had the SNMs genes in their tumors, respectively (FIG. 13A). Visualization of mutations' fingerprints of genes harboring the SNMs signatures of death from cancer phenotypes revealed that these genes isolated from clinical tumor samples appear “littered” with mutations, a vast majority of which is represented by the SNMs (FIG. 13B).

Interestingly, 11 of 18 (61%) death from cancer SNMs' signature genes are located near fifteen human-specific NANOG-binding sites [3], suggesting that these genes may represent genetic elements of the NANOG-regulatory network in the hESC. The placement of 15 human-specific NANOG-binding sites near 11 death from cancer SNMs' signature genes is significantly higher than could be expected by chance alone (p=9.95E-05; hypergeometric distribution test). This is in contrast to other human-specific transcription factor binding sites (CTCF; POU5F1; RNAPII), none of which manifest the significant placement enrichment near death from cancer SNMs' signature genes (data not shown). Notably, the changes of gene copy numbers of all of these 18 genes seem associated with poor long term survival of cancer patients (FIGS. 14A-14D), thus confirming the potential diagnostic and prognostic values of this gene panel using independent analytical end points for detection of gene-specific genetic alterations.

Next, the search for genes detection of SNMs in which is associated with increased likelihood of death from cancer was conducted employing multiple pan-cancer datasets (see below) to interrogate 127 genes significantly mutated in human cancer [23] and 177 genes listed in the catalogue of somatic mutations in cancer, COSMIC (cancersangerac.uk/cosmic/census). In total, 42 genes have been identified, which acquired somatic non-silent mutations in clinical samples of malignant tumors and the presence of these mutations is associated with significantly increased likelihood of poor therapy outcomes and death from cancer (Data Set S3 (Tables 15-17)). Notably, 33 of 42 (78.6%) of genes harboring mutations' fingerprints of death from cancer phenotypes constitute members of SCARs-associated genomic networks (FIG. 16 and Data Set S3 (Tables 15-17)).

Validation analyses of SNMs' signatures associated with increased likelihood of death from cancer Detection of somatic non-silent mutations (SNMs) in genome-wide high-throughput experiments represents a significant experimental and analytical challenge. SNMs' calls are affected by numerous factors even during the processing of the same DNA samples. In addition to the technical factors, such as library preparation and sequencing platforms, differences in analytical and computational methodologies, such as mapping of sequencing reads and calling algorithms, the choice of the reference genome database, genome annotation, and target selection regions all contribute to the identification of SNMs. Finally, differences in ad-hoc pre/post data processing such as black lists of genes and samples may be a confounding factor. To account for these potential sources of variability, the significance of the associations between cancer patients' survival and SNMs calls were examined using the databases of somatic non-silent mutations calls reported by different research teams for pan-cancer datasets available at the UCSC Xena browser. In total, ten pan-cancer datasets comprising from 1,934 to 8,272 tumor samples were evaluated in this analysis (Data Set S3 (Tables 15-17)). All eighteen genes of the SNMs' death from cancer phenotype signature (FIGS. 5A-5D) were scored as statistically significant genes in at least two pan-cancer datasets (Data Set S3 (Tables 15-17)). Seventeen of eighteen SNMs' signature genes (94.4%) were identified in at least three datasets as statistically significant genes, SNMs' mutations in which were associated with the increased likelihood of death from cancer defined by the Kaplan-Meier analysis (Data Set S3 (Tables 15-17)). Similarly, detection of SNMs in 39 of 42 genes (92.9%) was associated with the significantly increased likelihood of death from cancer in at least two pan-cancer datasets (Data Set S3 (Tables 15-17)). Taken together, these observations seem to argue that identified herein genes represent promising candidate genetic markers that are sufficiently robust to justify definitive mutation target site-specific validation experiments and follow-up structural-functional and mechanistic studies.

Linear regression analyses of the clinical intractability of malignant tumors in patients diagnosed with multiple types of malignant tumors revealed striking evidence of associations between the likelihood of dying from cancer, cancer types, and the presence of SNMs' death from cancer signatures in tumors (FIGS. 5A-5D). In one analysis, cancer patients' survival data from TCGA Pan-cancer cohort of 28 cancer types were utilized to calculate the percent of death events for each cancer type. The resulting values were aligned with the percent of patients with the SNMs' death from cancer signatures in the corresponding groups of cancer patients and subjected to the linear regression analysis (FIG. 5C). In another analysis, age-adjusted cancer incidence and death rates (per 100,000 people) in the United States for 19 cancer types were obtained from the Center for Disease Control and Prevention (CDC) United States Cancer Statistics (USCS) report. The estimated death rates for each cancer type were calculated by multiplying the corresponding values of incidence rates and percent's of patients with the SNMs death from cancer signatures. The estimated death rate values were aligned with the actual death rates for the corresponding cancer types and subjected to the regression analysis (FIG. 5D). In both instances, the strikingly significant correlations were observed, strongly supporting the hypothesis that the presence of SNMs' signatures in tumors may represent a molecular signal of the increased likelihood of developing clinically lethal disease.

Collectively, present analyses indicate that molecular evidence of activation of defined genetic elements of SCARs-associated genomic networks in clinical tumor samples appears linked with the increased likelihood of manifestation of clinically lethal death from cancer phenotypes defined by the poor long-term survival of cancer patients after diagnosis and therapy of malignant tumors. The observed significant correlation of poor survival of cancer patients and copy number changes of genes constituting the master transcriptional regulators of SCARs activity and maintenance of the stemness networks in hESC, namely KLF4, LBP9, POU5F1, and NANOG, strongly support this hypothesis (FIGS. 14A-14E). These data suggest that activation of SCARs-associated genomic networks in cancer cells may provide selective growth and/or survival advantages and represent genetic signals of positive selection during malignant progression.

This conclusion is further supported by the analysis of the expression of proteins encoded by the SCARs-regulated genes in the clinical samples of the TCGA PANCAN12 cohort FIGS. 6A and 6B. All available protein expression data associated with the Kaplan-Meier survival curves were evaluated for 38 HERVH/LBP9-regulated genes. Notably, changes in the protein expression levels of 23 SCARs-regulated genes (60.5%) manifested significant associations with the long-term survival probability of cancer patients (Data Set 51 (Tables 4-9)). Examples of these highly significant associations are shown in FIGS. 6A and 6B, confirming the hypothesis that functional alterations of the SCARs-associated stemness genomic networks may play a role in clinically lethal disease progression in cancer patients.

Based on the results of present analyses, it has been concluded that TCGA-guided surveys of SCAR's networks in 12,093 clinical samples across all TCGA cohorts representing twenty-nine distinct types of human cancer revealed pan-cancer genomic signatures of clinically-lethal therapy resistant disease defined by the presence of somatic non-silent mutations (SNMs), gene-level copy number changes, transcripts' and proteins' expression of SCARs-regulated host genes. Reported in this communication genes represent promising candidate genetic markers of clinically lethal forms of human cancer that are sufficiently robust to justify definitive mutation target site-specific validation experiments and follow-up structural-functional and mechanistic studies.

Genome-wide mapping of defined genetic signatures of distinct SCAR's loci revealed marked expansion in the human genome of conserved protein domains encoded by the human-specific chimeric transcript.

Analysis of conserved protein domains within translated amino acid sequences encoded by human-specific SCARs-derived host/virus chimeric transcripts demonstrates that different SCARs' loci manifest distinct protein-coding signatures defined by the combinatorial patterns of conserved protein domains (FIGS. 2A-2M and FIGS. 11A-11K). Systematic BLAST analyses of individual SCAR's sequences demonstrate that mutations of viral sequences degraded the full coding potentials of functional viral proteins and only residual structures of certain conserved protein domains remain preserved (FIGS. 2A-2M and FIGS. 11A-11K). Notably, one of the most frequently represented conserved protein domains within translated amino acid sequences encoded by human-specific SCARs-derived host/virus chimeric transcripts is the GVQW amino acid sequence FIGS. 2A-3D. Because nucleotide sequences of distinct SCARs' loci encoding the GVQW amino acid sequence are readily distinguishable, it was possible to ascertain the numbers of the GVQW-encoding sequences in the human genome that were seeded by different SCARs loci. It has been hypothesized that this analysis may be useful for evaluation of the relative impact of expansion of different SCARs loci on spreading the GVQW domain across the human genome.

Genome-wide mapping of specific genetic signatures of distinct SCARs' loci encoding the conserved GVQW protein domain identified thousands of locus-specific genetic fingerprints scattered across the human genome, which were defined as nucleotide sequences having 100% sequence identity with no gaps or insertions compared with the parental SCAR's sequence FIGS. 3A-3D. Remarkably, this analysis revealed that the majority of DNA sequences encoding the GVQW conserved protein domain sequences in the human genome seems to originate from the human-specific chimeric transcripts derived from DNA sequences on chrY:278899 -284215 & chrX:278899 -284215 FIGS. 3A-3D. This expansion of specific SCARs-derived nucleotide sequences may have contributed to the marked enrichment of the GVQW conserved protein domains within the human proteome compared with other Great Apes FIGS. 3A-3D.

Further analysis revealed that zinc finger proteins represent one of the largest protein families in the human genome that harbor the GVQW domains. Therefore, it was of interest to determine whether expression of the zinc finger proteins harboring the GVQW domains is altered in malignant tumors from cancer patients with distinct long-term survival after therapy. Remarkably, this analysis demonstrates that changes of mRNA expression levels and gene copy numbers of zinc finger proteins harboring the GVQW domains appear to segregate cancer patients into sub-groups with markedly distinct treatment outcomes FIGS. 12A-12D. The observed patterns of changes in gene expression and gene copy numbers seem useful for identification of individuals with increased likelihood of therapy failure and death from cancer among patients diagnosed with prostate, breast, colon, rectal, and pancreatic cancers FIGS. 12A-12E. It will be of interest to determine experimentally what the function of the GVQW domain is and how the insertion of this domain into specific protein sequences affects the structural-functional properties of host proteins.

Remarkably, the gene-level copy number changes of all 21 zinc finger proteins with GVQW conserved protein domains and three SCARs network zinc finger protein genes (ZNF443; ZNF587; ZNF814) manifest highly significant associations with the poor prognosis and increased likelihood of death from cancer defined by the Kaplan-Meier survival analyses of the 12,093 clinical samples comprising TCGA Pan-cancer cohort FIGS. 4A-4D. These data strengthen the conclusion regarding the potential diagnostic and prognostic values of the zinc finger proteins containing the conserved GVQW domains for the clinical management of cancer patients and identification of individuals with the increased risk of therapy failure and disease progression.

Putative role of DNA repair pathways in creation of human-specific regulatory sequences encoded by endogenous human SCARs.

Mammalian cells have evolved to efficiently employ highly effective DNA repair pathways capable of patching DNA double-stranded brakes (DSBs) with almost any DNA molecules available in the vicinity of the lesions [24, 25]. Insertions of transposable element (TE)-derived DNA sequences (including DNA transposons and both LTR and non-LTR retrotransposons) at the site of DNA lesions appear to utilized by eukaryotic cells to repair DSBs [26-31]. An alternative model of TE-derived DNA capture, an endonuclease-independent L1 insertion mechanism at DNA DSBs repair sites has been proposed [27, 28, 30]. This pathway was initially observed in DNA repair-deficient rodent cell lines [27]. Subsequent reports indicated that this mechanism is likely to function in the human genome as well [28, 30-32]. It has been suggested that non-classical mechanisms of TE insertions may be associated with DSBs repair mediated by Alu elements [31] and HERV-K retroviruses [32]. It was of interest to ascertain whether SCARs activity may have contributed to the DNA repair in human cells.

A consensus signature feature of the non-classical TE-insertion mechanisms observed for various classes of retrotransposons is deletions of ancestral DNA sequences within the sites of insertions of TE-derived sequences. Human-specific deletions associated with TE-mediated DSBs are often extended for thousands base pairs of ancestral DNA sequences [31, 32]. To ascertain whether SCARs may have contributed to the DSBs repair pathways, candidate human-specific regulatory sequences (HSRS) encoded by endogenous human SCARs were identified and analyzed for the presence of human-specific gains (insertions) and losses (deletions) of regulatory DNA (Tables 1, 2). As expected, a majority of transcriptionally-active in human pluripotent stem cells HSRS (75.0%-79.5%) contains human-specific insertions (Table 2). Remarkably, the DNA sequence conservation analysis employing the LiftOver algorithm and Multiz Alignments of 20 mammals (17 primates) of the UCSC Genome Browser on Human December 2013 (GRCh38/hg38) Assembly (http://genome.ucsc.edu/cgi-bin/hgTracks?db=hg38&position=chr1%3A90820922-90821071&hgsid=441235989_eelAivpkubSY2AxzLhSXKL5ut7TN) revealed that 74.4%-88.6% of SCARs-encoded HSRS contain deletions of ancestral DNA sequences defined by the comparisons with the chimpanzee and bonobo genomes (Table 2). Notably, 40.0%-59.1% of SCARs-encoded HSRS contain large continuous human-specific losses of DNA segments exceeding 1,000 bp. in length. Some of the most extreme examples include the human-specific deletion of 27,843 bp. (hg38 coordinates: chr4:132,117,632-132,124,853) compared with chimpanzee's genome and the human-specific deletion of 81,108 bp. (hg38 coordinates: chr4:3,927,445-3,933,080) compared with bonobo's genome. Similarly, large human-specific deletions of 75,171 bp. (chr12:8,279,022-8,294,090), 35,326 bp. (chr4:3,927,445-3,933,080), and 71,036 bp. (chr1:112,809,666-112,826,054) were detected at different loci of SCAR's insertions compared with gorilla, orangutan and gibbon genomes, respectively.

Present analysis identified 101 transcriptionally active in human pluripotent stem cells SCARs-encoded human-specific regulatory loci that underwent multiple independent events of distinct human-specific DNA losses during primate's evolution (Table 2). Genomic coordinates of these 101 loci manifesting human-specific deletions' cascade patterns were identified by comparisons of human DNA sequences with the orthologous sequences of non-human primates using the UCSC Genome Browser tracks of the Multiz Alignments of 20 mammals (17 primates). In this analysis HSRS were defined as the genomic loci with human-specific deletions' cascade patterns when a continuous human-specific DNA sequence in the human genome manifests at least 2 distinct events of human-specific deletions compared to genomes of at least 2 different species of non-human primates, which were selected from the group comprising of chimpanzee, bonobo, gorilla, orangutan, and gibbon. Therefore, genomic loci manifesting human-specific deletions' cascade patterns appear to experience repeated losses of distinct continuous DNA segments over extended time periods during primates' evolution, which would be consistent with the mechanism of repetitive cycles of occurrence of DSBs and repair of DNA molecules mediated by the insertions of SCARs sequences at these genomic locations.

These distinctive structural features of human-specific SCAR's integration sites suggest that molecular mechanisms of the SCARs-associated DSBs repair may be similar to a backup DNA repair pathway known as an alternative non-homologous end-joining (Alt NHEJ), because the hallmark features of the repair junctions built by the Alt NHEJ pathway are large DNA deletions, insertions, and tracts of microhomology [33, 34]. Collectively, these data support the hypothesis that the Alt NHEJ pathway of DSBs repair may have contributed to the insertions of SCARs at specific genomic locations, which resulted in creation of HSRS transcriptionally active in human pluripotent stem cells FIGS. 7A-7D.

### DESCRIPTION OF POTENTIAL BIOLOGICAL, PATHOPHYSIOLOGICAL, DIAGNOSTIC, AND THERAPEUTIC IMPLICATIONS

Implications for the Liquid Biopsy Applications

Observations that malignant tumors shed cell-free fragments of DNA into the bloodstream as a result of apoptotic and/or necrotic death of cancer cells pave the way for the disclosure and rapid introduction into experimental and clinical cancer research the concept of a liquid biopsy based on the analysis of circulating cell-free (cfDNA) derived from cancer cells. The consensus view emerged that the load of cfDNA derived from cancer cells appear to correlate with tumor staging and prognosis [Diaz L A Jr, Bardelli A. Liquid Biopsies: Genotyping Circulating Tumor DNA. J Clin Oncol. 2014;32: 579-86; Haber, D. A. & Velculescu, V. E. Blood-Based Analyses of Cancer: Circulating Tumor Cells and Circulating Tumor DNA. Cancer Discov. 2014; 4: 650-661; Bettegowda, C. et al. Detection of circulating tumor DNA in early- and late-stage human malignancies. Sci. Transl. Med. 2014; 6: 224ra24; Newman A M, Bratman S V, To J, Wynne J F, Eclov N C, Modlin L A, Liu C L, Neal J W, Wakelee H A, Merritt R E, Shrager J B, Loo B W Jr, Alizadeh A A, Diehn M. An ultrasensitive method for quantitating circulating tumor DNA with broad patient coverage. Nat. Med. Nat Med. 2014; 20: 548-54; Dawson S J, Tsui D W, Murtaza M, Biggs H, Rueda O M, Chin S F, Dunning M J, Gale D, Forshew T, Mahler-Araujo B, Rajan S, Humphray S, Becq J, Halsall D, Wallis M, Bentley D, Caldas C, Rosenfeld N. Analysis of circulating tumor DNA to monitor metastatic breast cancer. N. Engl. J. Med. 2013; 368: 1199-209; Garcia-Murillas I, Schiavon G, Weigelt B, Ng C, Hrebien S, Cutts R J, Cheang M, Osin P, Nerurkar A, Kozarewa I, Garrido J A, Dowsett M, Reis-Filho J S, Smith I E, Turner N C. Mutation tracking in circulating tumor DNA predicts relapse in early breast cancer. Sci Transl Med. 2015;7: 302ra133]. Most recent advances in the next generation sequencing technology markedly improved the sensitivity, specificity, and accuracy of the analysis of tumor-derived DNA. In principle, the state of the art next generation sequencing techniques have allowed for genotyping of tumor-derived cfDNA for somatic genomic alterations which were previously possible to document only by the direct analysis of cancer cells. The ability to readily detect and reliably quantify highly heterogeneous spectrum of mutations in individual tumors using cfDNA-based assays has proven highly efficient in tracking dynamics of tumor evolution in real time that can be used for a variety of translational applications facilitating the clinical implementation of the concept of personalized disease management in cancer patients.

Despite the perceived great promise for multiple translational applications, the liquid biopsy technology in its current form has significant limitations. These limitations are particularly apparent when the intended uses of the liquid biopsy for diagnosis of the early-stage solid tumors or prospective identification of therapeutically actionable mutations of cancer driver genes are carefully considered. In its current form, the liquid biopsy is primarily utilized for in-depth high-resolution sequencing of cfDNA extracted from blood samples (plasma or serum) with the primary intent to reliably detect somatic mutations in pre-selected sets of cancer driver genes. It seems reasonable to expect that tumor vascularization would be required for cancer cell-derived cfDNA to appear in blood. However, it is well established that the early stages of development of essentially all solid tumors in cancer patients are characterized by the lack of the need for vascularization and, indeed, represent the avascular stage of tumor development and progression for many years with the sufficient nutrient supply by diffusion. In this context, the appearance of tumor-derived cfDNA in blood should be regarded as the evidence of tumor vascularization and a molecular signal of increased likelihood of malignant progression toward metastatic disease. Consistent with this line of reasoning, tumor-derived cfDNA is reliably and reproducibly detected in blood of >90% of cancer patients with advanced solid tumors, whereas the detection rate drops to ˜50% (or less) in blood from patients diagnosed with the early-stage cancers. Importantly, it is almost certain that further improvements in the analytical performance of the next generation sequencing technology would not dramatically change these realities.

It appears that the consensus view is that the primary origin of the cancer cell-derived cfDNA is from tumor cells undergoing apoptotic and/or necrotic death. There are no credible evidence consistently demonstrating that the origin of tumor-derived cfDNA extracted from blood samples is from viable actively dividing cancer cells or tumor growth-sustaining minority sub-populations of cancer cells such as cells of cancer origin, tumor-initiating cells, or cancer stem cells. Therefore, it is reasonable to believe that mutational signatures of tumor-derived cfDNA extracted from blood of cancer patients represent the past history of tumor evolution and there is no credible way to discern the real time mutational status or to predict the future of tumor evolution based on the genetic information extracted from dead cancer cells.

Most recent analysis of genome-wide mutational dynamics during tumor evolution at the single-nucleus resolution revealed that somatic point mutations, in contrast to aneuploidies, evolved gradually and generated extensive clonal diversity [Wang Y, Waters J, Leung M L, Unruh A, Roh W, Shi X, Chen K, Scheet P, Vattathil S, Liang H, Multani A, Zhang H, Zhao R, Michor F, Meric-Bernstam F, Navin N E. Clonal evolution in breast cancer revealed by single nucleus genome sequencing. Nature. 2014; 512: 155-160]. Targeted single-molecule sequencing conclusively demonstrated that many of diverse point mutations detected in tumors occur at frequency <10% of tumor cell populations. In striking contrast, aneuploid rearrangements appeared early in tumor evolution and remained highly stable during the clonal expansion [Wang, Y., et al. Clonal evolution in breast cancer revealed by single nucleus genome sequencing. Nature. 2014; 512: 155-160]. This contribution links development of aneuploidies with aberrant activity of SCARs networks and demonstrates that gene expression signatures of activated SCAR's pathway (s) can be detected in clinical samples of cancer precursor lesions, localized tumors, and metastatic cancers. Collectively, these observations strongly argue that activation of SCARs networks and associated genomic aberrations are likely to occur in the cancer precursor cells and continually persist throughout tumor evolution and progression toward metastatic disease. Therefore, detection of identified herein SCARs sequences, SCAR/host gene hybrid sequences, SCARs-regulated protein coding genes and non-coding RNA sequences will open the remarkable opportunities for diagnostic, prognostic, therapy selection, and disease management applications utilizing the liquid biopsy technology.

Cell-free macromolecules, including nucleic acids and proteins, are often reside in nano-scale size particles called exosomes. Packaging of DNA and RNA molecules in the exosomes appears to protect them from degradation by extracellular nucleases and the biologically active nucleic acid molecules such as microRNAs and lincRNA appears to remain stable. Therefore, the sample preparation protocols for liquid biopsy analyses would likely to benefit from the inclusion of the exosome enrichment and purification step.

Putative Role of SCAR's Sequences in DNA Repair and Increased Survival of Metastatic Cancer Cells

Present analyses suggest a plausible biological role for SCARs in DNA repair that may override the potentially harmful effects of retrotransposon-driven mutations by providing the immediate survival and fitness advantages to host cells, which would be particularly beneficial for immortal cancer cells. Despite relatively high activity of DNA repair pathways, hESCs exhibit increased sensitivity to radiation-induced DNA damage and apoptosis [35, 36]. It has been suggested that increased sensitivity to apoptosis of hESC is due to low apoptotic threshold in response to DNA damage [36]. In striking contrast, previously reported experimental and clinical evidence of activation of stemness pathways in therapy resistant malignant tumors, highly metastatic cancer cells, and circulating tumor cells consistently demonstrated genetic and phenotypic associations with manifestations of markedly increased resistance to apoptosis induced by various biologically-relevant micro-environmental changes and different chemical perturbations [37-51]. These important biological distinctions, which are defined by the underlying differences of genomic architectures between normal human pluripotent stem cells and highly malignant populations of tumor cells with activated stemness genetic networks, are likely responsible for relentless growth, self-renewal, survival, and tumor-initiating abilities of cancer stem cells. Continuing transcriptional activity of SCARs in tumor cells may represent a constant potentially deadly threat despite their apparent structural deficiencies to encode the functional viral genomes. There are many thousand variants of SCARs' sequences integrated in the human genome, suggesting that many mutations of SCARs' genes can be repaired by recombination with endogenous copies of SCARs' sequences. Consistent with this hypothesis, it has been demonstrated that introduction of mutant retroviruses carrying a lethal deletion in an essential viral gene can result in spread of revertant viruses that repaired the mutation by homologous recombination with endogenous DNA sequences [52].

Genomic Networks of Stem Cell-Associated Retroviruses Harbor Signatures of Clinically Intractable Malignant Tumors

Present analysis of SCARs and associated stemness genomic networks was focused on genetic loci harboring human-specific insertions and/or deletions that may have contributed to development of human-specific regulatory networks and pathways. One of the primary line of reasoning for the choice of this strategy is based on the apparent major differences in the cancer incidence between humans and nonhuman primates that have been documented extensively. Prostate carcinoma is essentially nonexistent and lung cancer is very rare in nonhuman primates (53-58). Overall, the incidence rate of common cancers, including breast, prostate, lung, colon, ovary, pancreas, and stomach, is estimated in the range of ˜2% to 4% (53-57). Unique to human phenotypic effects of human-specific regulatory loci and pathways operating within the circuitry of stemness genomic networks may have contributed to these dramatic species-specific differences in the cancer incidence.

Based this idea, the initial analysis was focused on the host/virus chimeric transcripts which harbor human-specific SCARs insertions (Tables 1-3; FIGS. 1A-1H). Observed changes of mRNA expression levels and gene copy numbers of SCARs-targeted protein-coding genes with human-specific retroviral insertions comprising structural elements of host/virus chimeric transcripts support the hypothesis that different SCAR's activation patterns are associated with significantly distinct long term survival of cancer patients.

Next, the analysis of conserved protein domains within translated amino acid sequences encoded by human-specific SCARs-derived host/virus chimeric transcripts was carried out. It demonstrates that different SCARs' loci manifest distinct protein-coding signatures defined by the combinatorial patterns of conserved protein domains FIGS. 2A-2M and FIGS. 11A-11K. It has been observed that one of the most frequently represented conserved protein domains within translated amino acid sequences encoded by human-specific SCARs-derived host/virus chimeric transcripts is the GVQW amino acid sequence FIGS. 2A-3D. Using defined SCARs-locus-specific signatures of nucleotide sequence encoding GVQW domains, it has been determined that the origin of a majority of DNA sequences encoding the GVQW amino acid sequences in the human genome is from the human-specific chimeric transcripts encoded by DNA sequences on chrY:278899 -284215 & chrX:278899 -284215 FIGS. 3A-3D. The spreading of SCARs-derived nucleotide sequences appears to result in the marked expansion of the specific GVQW-encoding DNA sequences and ˜10-fold enrichment of the GVQW conserved protein domains within the human proteome compared with other Great Apes FIGS. 3A-3D. These data strongly argue that one of the biologically-significant consequences of the continuing SCARs activity is the seeding of nucleotide sequences encoding specific conserved protein domains throughout the human genome.

Remarkably, subsequent analysis demonstrates that changes of mRNA expression levels and gene copy numbers of zinc finger proteins harboring the GVQW domains segregate cancer patients into sub-groups with markedly distinct treatment outcomes (FIGS. 4A-4D and FIGS. 12A-12E). The observed patterns of changes in gene expression and copy numbers seem to segregate individuals with increased likelihood of therapy failure and death from cancer among patients diagnosed with prostate, breast, colon, rectal, and pancreatic cancers (FIGS. 12A-12E). Among patients diagnosed with prostate and rectal cancers, it appears possible to identify the good prognosis sub-group of patients comprising of individuals with ˜100% survival probability more than 10 years after diagnosis and therapy (FIGS. 12A-12E), which may have a highly significant clinical implications for individualized, evidence-based disease management decision making process.

To determine whether genetic signatures of SCARs activity may be potentially useful for diagnostic and prognostic applications, the SCAR's genomic networks were systematically searched for genes that acquired somatic non-silent mutations, detection of which in tumor samples is associated with increased likelihood of death from cancer. A total of 42 human genes have been identified in this contribution that acquired somatic non-silent mutations in clinical tumor samples across all TCGA cohorts and presence of these mutations in malignant tumors seems associated with significantly increased likelihood of death from cancer (FIGS. 5A-5D; FIG. 16; Tables 15-17). A significant majority of genes (33 of 42; 78.6%) harboring mutations' fingerprints of death from cancer phenotypes constitute members of SCARs-associated genomic networks (FIG. 16 and Tables 15-17), thus confirming that molecular evidence of activation of defined genetic elements of SCARs-associated stemness genomic networks in clinical tumor samples appears linked with the increased likelihood of manifestation of clinically lethal death from cancer phenotypes defined by the Kaplan-Meier survival analysis. Significantly, it has been observed that more than 70% of all cancer death events occurred in the poor prognosis patients' sub-group defined by the death from cancer SNMs' signature (FIGS. 5A-5D).

One of the significant conclusions reported in this contribution is based on the observations that detection of molecular evidence of altered activities of defined genetic elements of SCARs-associated stemness genomic networks in clinical tumor samples appears associated with the increased likelihood of clinical manifestation of disease progression defined by the poor long-term survival of cancer patients after diagnosis and therapy of malignant tumors. Observations of engagements of specific genes within SCARs networks in tumors are based on detection of somatic non-silent mutations and changes of gene copy numbers, suggesting that altered activities of SCARs-associated genomic networks in cancer cells may provide selective growth and/or survival advantages and represent genetic signals of positive selection during malignant progression. Significantly, the clinical intractability of malignant disease, which was ascertained based on the long-term survival of patients diagnosed with twenty-eight cancer types, is directly correlated with the percentage of cancer patients whose tumors harbor somatic non-silent mutations' signatures. Therefore, reported herein genetic correlates of death from cancer phenotypes may represent highly attractive targets for development of novel diagnostic, prognostic, and therapeutic applications directed against intractable human malignancies.

Consistent with the idea that the human-specific structural-functional features of SCAR's genomic networks may play unique roles in both physiology and pathology of H. sapiens, it has been reported that the HERV-H transcriptome has recently evolved in humans under the influence of directional selection and is likely to exert detectable fitness effects on the host since the chimp-human split (59). Explorations of biologically significant functions of SCARs in the pathological and physiological conditions should not focus exclusively on the detection and isolation of infectious viral particles. Like many other HERV families, the majority of SCAR's sequences accumulated multiple mutations and deletions during evolution and no HERV sequence has been shown to be replication-competent and infectious.

In human genome the HERV-K family comprises 91 proviruses with full or partial coding capacity of retroviral proteins and 944 solo LTRs (60). Collectively, HERV-K proviruses maintain open reading frames for all retroviral genes needed for infectivity and potential recombination among only three HERV-K proviruses could facilitate the production of an infectious retrovirus (61). However, the new conclusive evidence of significant impact of SCARs-derived retroviral sequences on development of cancer in humans may not necessarily require the isolation of infectious virus and establishing a correlation between the viral infection and cancer incidence. The pathologically significant effects of retroviral sequences may arise from many different mechanisms of their biological activities and can be demonstrated as the following experimental evidence (62):

Presence of New, Cancer-Specific Integration Sites of Retroviruses;

Consistent regulatory targeting of one or a few host genes in many different tumors;

Oncogenic actions of protein products of retroviral genes (env; rec; np9);

Targeted regulatory effects on expression of host genes due to contributions of new splice donor or acceptor sites, alternative promoters, and transcription regulatory sites.

In addition, presence of multiple SCAR's sequences on the same and/or different chromosomes is likely to facilitate the chromosomal rearrangements due to recombination events between the genomic loci within the permissive chromatin context.

Present analyses suggest that epigenetic activation of silenced SCAR's loci in differentiated cells may establish a cancer susceptibility state in a cell by engaging stemness regulatory networks. It seems plausible to argue that subsequent mutagenesis and selection of cancer driver genes occur in cells with SCARs-activated stemness networks, which would explain why nearly two-third of high confidence cancer drivers and COSMIC genes appear regulated by SCARs in hESC (see above). The central postulate of this hypothesis predicts the presence of pre-cancerous differentiated cells with SCARs-activated stemness networks that may serve as a precursor of cancer stem cells, emergence of which would subsequently fuel tumor growth, cancer progression, metastasis, and development of clinically intractable malignancies.

### Materials and Methods

Data Sources and Analytical Protocols

Solely publicly available datasets and resources were used for this analysis as well as methodological approaches and a computational pipeline validated for discovery of primate-specific gene and human-specific regulatory loci [3; 63-68]. The individual genetic elements comprising the SCARs-associated stemness genomic networks, including HERVH/LBP9-regulated genes identified in the hESC using shRNA experiments [19], were obtained from the recently published contributions reporting transcriptionally active SCARs loci [12; 16-20], host/virus chimeric transcripts [18-20], and human-specific transcription factor binding sites (TFBS) seeded in the hESC genome by SCARs [3].

The most recent beta release of web-based tools of The Cancer Genome Atlas (TCGA) project, the UCSC Xena (http://xena.ucsc.edu/), associated clinical data, and multiple functional cancer genomics' end points identified in thousands tumor samples were utilized to explore, analyze, and visualize the clinically-relevant patterns of gene expression, somatic non-silent mutations, and gene copy numbers of individual genetic elements of the SCARs-associated stemness genomic networks by interrogating the comprehensive functional cancer genomics datasets of more than twelve thousands annotated clinical tumor samples (https://genomecancer.soe.ucsc.edu/proj/site/xena/datapages/). Pan-cancer signatures of gene expression, somatic non-silent mutations, and copy number changes associated with increased likelihood of death from cancer were identified by interrogation of two TCGA Pan-Cancer databases, comprising 5,158 clinical samples across 12 TCGA cohorts (PANCAN12 study of 12 distinct cancer types) and 12,088 clinical samples across all TCGA cohorts (https://genomecancer.soe.ucsc.edu/proj/site/xena/datapages/).

The sequence conservation analysis is based on the University of California Santa Cruz (UCSC) LiftOver algorithm for conversion of the coordinates of human blocks to corresponding non-human genomes using chain files of pre-computed whole-genome BLASTZ alignments with a MinMatch of 0.95 and other search parameters in default setting (http://genome.ucsc.edu/cgi-bin/hgLiftOver). Extraction of BLASTZ alignments by the LiftOver algorithm for a human query generates a LiftOver output “Deleted in new”, which indicates that a human sequence does not intersect with any chains in a given non-human genome. This indicates the absence of the query sequence in the subject genome and was used to infer the presence or absence of the human sequence in the non-human reference genome. Human-specific regulatory sequences were manually curated to validate their identities and genomic features using a BLAST algorithm and the latest releases of the corresponding reference genome databases for time periods between April, 2013 and October, 2015.

Considerations of the putative functionally-significant regulatory effects of SCARs on host genes were based, in part, on the results of the genome-wide proximity placement analyses of the corresponding candidate regulatory elements and target genes. The quantitative limits of proximity during the proximity placement analyses were defined based on several metrics. One of the metrics was defined using the genomic coordinates placing human-specific regulatory sequences closer to putative target protein-coding or IncRNA genes than experimentally defined distances to the nearest targets of 50% of the regulatory proteins analyzed in hESCs [69]. For each gene of interest, specific HSGRL were identified and tabulated with a genomic distance between HSGRL and a putative target gene that is smaller than the mean value of distances to the nearest target genes regulated by the protein-coding TFs in hESCs. The corresponding mean values for protein-coding and IncRNA target genes were calculated based on distances to the nearest target genes for TFs in hESC reported by Guttman et al. [69]. In addition, the proximity placement metrics were defined based on co-localization within the boundaries of the same topologically associating domains (TADs) and the placement enrichment pattern of human-specific NANOG-binding sites (HSNBS) located near the 251 neocortex/prefrontal cortex-associated genes [70]. The placement enrichment analysis of HSNBS identified the most significant enrichment at the genomic distances less than 1.5 Mb with a sharp peak of the enrichment p value at the genomic distance of 1.5 Mb [70].

Comprehensive databases of individual regulatory elements and chromatin regulatory domains identified in the hESC genome were considered in this study. Genomic coordinates of 3,127 topologically-associating domains (TADs) in hESC; 6,823 hESC-enriched enhancers; 6,322 conventional and 684 super-enhancers (SEs) in hESC; 231 SEs and 197 super-enhancers domains (SEDs) in mESC were reported in the previously published contributions [2; 71-74]. Species-specific datasets of NANOG-, POU5F1-, and CTCF-binding sites and human-specific TFBS in hESCs were reported previously [3; 4] and are publicly available. RNA-Seq datasets were retrieved from the UCSC data repository site (http://genome.ucsc.edu/; [75]) for visualization and analysis of cell type-specific transcriptional activity of defined genomic regions. A genome-wide map of the human methylome at single-base resolution was reported previously [76; 77] and is publicly available (http://neomorph.salk.edu/human_methylome). The histone modification and transcription factor chromatin immunoprecipitation sequence (ChIP-Seq) datasets for visualization and analysis were obtained from the UCSC data repository site (http://genome.ucsc.edu/; [78]). Genomic coordinates of the RNA polymerase II (PIO-binding sites, determined by the chromatin integration analysis with paired end-tag sequencing (ChIA-PET) method, were obtained from the saturated libraries constructed for the MCF7 and K562 human cell lines [79]. The density of TF-binding to a given segment of chromosomes was estimated by quantifying the number of protein-specific binding events per 1-Mb and 1-kb consecutive segments of selected human chromosomes and plotting the resulting binding site density distributions for visualization. Visualization of multiple sequence alignments was performed using the WebLogo algorithm (http://weblogo.berkeley.edu/logo.cgi). Consensus TF-binding site motif logos were previously reported [4; 80; 81].

The assessment of conservation of HSGRL in individual genomes of 3 Neanderthals, 12 Modern Humans, and the 41,000-year old Denisovan genome [82; 83] was carried-out by direct comparisons of corresponding sequences retrieved from individual genomes and the human genome reference database (http://genome.ucsc.edu/Neandertal/).

Nucleotide sequences of human-specific chimeric transcripts were translated into amino acid sequences and subjected to the protein alignment analyses using the protein BLAST algorithm (http://blast.ncbi.nlm.nih.gov/Blast.cgi?PROGRAM=blastp&BLAST_PROGRAMS=blastp&PAGE_TYPE=BlastSearch&SHOW_DEFAULTS=on&LINK_LOC=blasthome) and associated web-based tools for identification and visualization of conserved protein domains (http://www.ncbi.nlm.nih.gov/Structure/cdd/wrpsb.cgi?RlD=3HZ5BMES01R&mode=all), which were described in details elsewhere [84, 85].

Age-adjusted cancer incidence and death rates in the United States were obtained from the Center for Disease Control and Prevention (CDC) United States Cancer Statistics (USCS) report:

U.S. Cancer Statistics Working Group. United States Cancer Statistics: 1999-2012 Incidence and Mortality Web-based Report. Atlanta: U.S. Department of Health and Human Services, Centers for Disease Control and Prevention and National Cancer Institute; 2015. Available at: www.cdc.gov/uscs .

Statistical Analyses of the Publicly Available Datasets

All statistical analyses of the publicly available genomic datasets, including error rate estimates, background and technical noise measurements and filtering, feature peak calling, feature selection, assignments of genomic coordinates to the corresponding builds of the reference human genome, and data visualization, were performed exactly as reported in the original publications and associated references linked to the corresponding data visualization tracks (http://genome.ucsc.edu/ and http://xena.ucsc.edu/). Any modifications or new elements of statistical analyses are described in the corresponding sections of the Results. Statistical significance of the Pearson correlation coefficients was determined using GraphPad Prism version 6.00 software. The significance of the differences in the numbers of events between the groups was calculated using two-sided Fisher's exact and Chi-square test, and the significance of the overlap between the events was determined using the hypergeometric distribution test [86].

### REFERENCES

1. Santoni, F.A., Guerra, J., and Luban, J. HERV-H RNA is abundant in human embryonic stem cells and a precise marker for pluripotency. Retrovirology 2012; 9: 111.
2. Xie W, Schultz M D, Lister R, Hou Z, Rajagopal N, Ray P, Whitaker J W, Tian S, Hawkins R D, Leung D, Yang H, Wang T, Lee A Y, Swanson S A, Zhang J, Zhu Y, Kim A, Nery J R, Urich M A, Kuan S, Yen C A, Klugman S, Yu P, Suknuntha K, Propson N E, Chen H, Edsall L E, Wagner U, Li Y, Ye Z, Kulkarni A, Xuan Z, Chung W Y, Chi N C, Antosiewicz-Bourget J E, Slukvin I, Stewart R, Zhang M Q, Wang W, Thomson J A, Ecker J R, Ren B. Epigenomic analysis of multilineage differentiation of human embryonic stem cells. Cell 2013. 153: 1134-1148.
3. Glinsky, G V. Transposable Elements and DNA Methylation Create in Embryonic Stem Cells Human-Specific Regulatory Sequences Associated with Distal Enhancers and Noncoding RNAs. Genome Biol Evol. 2015; 7: 1432-54.
4. Kunarso, G, Chia, N Y, Jeyakani, J, Hwang, C, Lu, ., Chan, Y S, Ng, H H, and Bourque, G. Transposable elements have rewired the core regulatory network of human embryonic stem cells. Nat Genet. 2010; 42: 631-634.
5. Kelley, D, and Rinn, J. Transposable elements reveal a stem cell-specific class of long noncoding RNAs. Genome Biol. 2012; 13: R107.
6. Glinsky G V. Endogenous human stem cell-associated retroviruses. BioRxiv 2015; doi: http://dx.doi.org/10.1101/024273
7. Glinsky G V. SCARs: endogenous human stem cell-associated retroviruses and therapy-resistant malignant tumors. arXiv preprint 2015; arXiv:1508.02022 http://arxiv.org/abs/1508.02022
8. Glinsky G V. Viruses, stemness, embryogenesis, and cancer: a miracle leap toward molecular definition of novel oncotargets for therapy-resistant malignant tumors? Oncoscience 2015; 2: 751-754.
9. Glinsky G V. Activation of endogenous human Stem Cell-Associated Retroviruses and therapy-resistant phenotypes of malignant tumors. 2016. In revision.
10. Smith Z D, Chan M M, Humm KC, Karnik R, Mekhoubad S, Regev A, Eggan K, Meissner A. DNA methylation dynamics of the human preimplantation embryo. Nature 2014; 511: 611-615.
11. Fort A, Hashimoto K, Yamada D, Salimullah M, Keya C A, Saxena A, Bonetti A, Voineagu I, Bertin N, Kratz A, Noro Y, Wong C H, de Hoon M, Andersson R, Sandelin A, Suzuki H, Wei C L, Koseki H; FANTOM Consortium, Hasegawa Y, Forrest A R, Carninci P. Deep transcriptome profiling of mammalian stern cells supports a regulatory role for retrotransposons in pluripotency maintenance. Nature Genet. 2-14; 46: 558-566.
12. Lu X, Sachs F, Ramsay L, Jacques P E, Goke J, Bourque G, Ng H H. The retrovirus HERVH is a long noncoding RNA required for human embryonic stem cell identity. Nat Struct Mol Biol. 2014; 21:423-425.
13. Ohnuki M, Tanabe K1, Sutou K, Teramoto I, Sawamura Y, Narita M, Nakamura M, Tokunaga Y, Nakamura M, Watanabe A, Yamanaka S, Takahashi K. Dynamic regulation of human endogenous retroviruses mediates factor-induced reprogramming and differentiation potential. Proc Natl Acad Sci USA. 2014. 111:12426-31.
14. Koyanagi-Aoi M, Ohnuki M, Takahashi K, Okita K, Noma H, Sawamura Y, Teramoto I, Narita M, Sato Y, Ichisaka T, Amano N, Watanabe A, Morizane A, Yamada Y, Sato T, Takahashi J, Yamanaka S. Differentiation-defective phenotypes revealed by large-scale analyses of human pluripotent stem cells. Proc Natl Acad Sci USA. 2013; 110: 20569-74.
15. Marchetto M C, Narvaiza I, Denli A M, Benner C, Lazzarini T A, Nathanson J L, Paquola A C, Desai K N, Herai R H, Weitzman M D, Yeo G W, Muotri A R, Gage F H. (2013). Differential LINE-1 regulation in pluripotent stem cells of humans and other great apes. Nature 503: 525-529.
16. Xue Z, Huang K, Cai C, Cai L, Jiang C Y, Feng Y, Liu Z, Zeng Q, Cheng L, Sun Y E, Liu J Y, Horvath S, Fan G. Genetic programs in human and mouse early embryos revealed by single-cell RNA sequencing. Nature 2013; 500: 593-597.
17. Yan L, Yang M, Guo H, Yang L, Wu J, Li R, Liu P, Lian Y, Zheng X, Yan J, Huang J, Li M, Wu X, Wen L, Lao K, Li R, Qiao J, Tang F. Single-cell RNA-Seq profiling of human preimplantation embryos and embryonic stem cells. Nat Struct Mol Biol 2013; 20: 1131-1139.
18. Goke J, Lu X, Chan Y S, Ng H H, Ly L H, Sachs F, Szczerbinska I. Dynamic transcription of distinct classes of endogenous retroviral elements marks specific populations of early human embryonic cells. Cell Stem Cell 2015; 16: 135-141.
19. Wang J, Xie G, Singh M, Ghanbarian A T, RaskóT, Szvetnik A, Cai H, Besser D, Prigione A, Fuchs N V, Schumann G G, Chen W, Lorincz M C, Ivics Z, Hurst LD, Izsvák Z. Primate-specific endogenous retrovirus-driven transcription defines naive-like stem cells. Nature 2014; 516: 405-9.
20. Grow E J, Flynn R A, Chavez S L, Bayless N L, Wossidlo M, Wesche D J, Martin L, Ware C B, Blish C A, Chang H Y, Pera R A, Wysocka J. Intrinsic retroviral reactivation in human preimplantation embryos and pluripotent cells. Nature 2015; 522: 221-5.
21. Robbez [symbol] Masson L, Rowe H M. Retrotransposons shape species [symbol] specific embryonic stem cell gene expression. Retrovirology 2015; 12: 45.
22. Tamborero D1, Gonzalez-Perez A, Perez-Llamas C, Deu-Pons J, Kandoth C, Reimand J, Lawrence M S, Getz G, Bader G D, Ding L, Lopez-Bigas N. Comprehensive identification of mutational cancer driver genes across 12 tumor types. Sci Rep. 2013; 3: 2650.
23. Hoadley K A, Yau C, Wolf D M, Cherniack A D, Tamborero D, Ng S, Leiserson M D, Niu B, McLellan M D, Uzunangelov V, Zhang J, Kandoth C, Akbani R, Shen H, Omberg L, Chu A, Margolin A A, Van't Veer L J, Lopez-Bigas N, Laird P W, Raphael B J, Ding L, Robertson A G, Byers L A, Mills G B, Weinstein J N, Van Waes C, Chen Z, Collisson E A; Cancer Genome Atlas Research Network, Benz C C, Perou C M, Stuart J M. Multiplatform analysis of 12 cancer types reveals molecular classification within and across tissues of origin. Cell 2014; 158: 929-44.
24. Yu, X. and Gabriel, A. Patching broken chromosomes with extranuclear cellular DNA. Mol. Cell 1999; 4: 873-881.
25. Lin, Y. and Waldman, A.S. Promiscuous patching of broken chromosomes in mammalian cells with extrachromosomal DNA. Nucleic Acids Res. 2001; 29: 3975-3981.
26. Teng, S.C., Kim, B. and Gabriel, A. Retrotransposon reverse transcriptase-mediated repair of chromosomal breaks. Nature 1996; 383: 641-644.
27. Morrish, T.A., Gilbert, N., Myers, J.S., Vincent, B.J., Stamato, T.D., Taccioli, G.E., Batzer, M.A. and Moran, J.V. DNA repair mediated by endonuclease-independent LINE-1 retrotransposition. Nat. Genet. 2002; 31: 159-165.
28. Morrish T A, Garcia-Perez J L, Stamato T D, Taccioli G E, Sekiguchi J, Moran J V. Endonuclease-independent LINE-1 retrotransposition at mammalian telomeres. Nature. 2007; 446: 208-12.
29. lchiyanagi, K., Nakajima, R., Kajikawa, M. and Okada, N. (2007) Novel retrotransposon analysis reveals multiple mobility pathways dictated by hosts. Genome Res. 2007; 17: 33-41.
30. Sen, S.K., Huang, C.T., Han, K., Batzer, M.A. Endonuclease-independent insertion provides an alternative pathway for L1 retrotransposition in the human genome. Nucleic Acids Res. 2007; 35: 3741-3751.
31. Srikanta D, Sen S K, Huang C T, Conlin E M, Rhodes R M, et al. An alternative pathway for Alu 63 retrotransposition suggests a role in DNA double strand break repair. Genomics 2009; 93: 205-212.
32. Shin W, Lee J, Son S-Y, Ahn K, Kim H-S, Han, K. Human-specific HERVK insertion causes genomic variations in the human genome. PLoS ONE 2013; 8: e60605.
33. Nussenzweig A, Nussenzweig M C. A backup DNA repair pathway moves to the forefront. Cell. 2007; 131: 223-225.
34. Iliakis G. Backup pathways of NHEJ in cells of higher eukaryotes: cell cycle dependence. Radiother Oncol. 2009; 92: 310-315.
35. Bogomazova A N, Lagarkova M A, Tskhovrebova L V, Shutova M V, Kiselev S L. Error-prone nonhomologous end joining repair operates in human pluripotent stem cells during late G2. Aging (Albany NY). 2011; 3: 584-96.
36. Fan J, Robert C, Jang Y Y, Liu H, Sharkis S, Baylin S B, Rassool F V. Human induced pluripotent cells resemble embryonic stem cells demonstrating enhanced levels of DNA repair and efficacy of nonhomologous end-joining. Mutat Res. 2011; 713: 8-17.
37. Glinsky G V, Glinskii A B, Berezovskaya O. Microarray analysis identifies a death-from-cancer signature predicting therapy failure in patients with multiple types of cancer. Journal of Clinical Investigation 2005; 115: 1503-21.
38. Glinsky G V. Death-from-cancer signatures and stem cell contribution to metastatic cancer. Cell Cycle 2005; 4: 1171-5.
39. Glinsky, G V. Genomic models of metastatic cancer: Functional analysis of death-from-cancer signature genes reveals aneuploid, anoikis-resistant, metastasis-enabling phenotype with altered cell cycle control and activated Polycomb Group (PcG) protein chromatin silencing pathway. Cell Cycle, 2006; 5: 1208-1216.
40. Berezovska, O P, Glinskii, A B, Yang, Z, Li, X-M, Hoffman, R M, Glinsky, G V. Essential role of the Polycomb Group (PcG) protein chromatin silencing pathway in metastatic prostate cancer. Cell Cycle, 2006; 5: 1886-1901.
41. Glinskii A B, Smith B A, Jiang P, Li X M, Yang M, Hoffman R M, Glinsky G V. Viable circulating metastatic cells produced in orthotopic but not ectopic prostate cancer models. Cancer Res. 2003; 63: 4239-43.
42. Berezovskaya O, Schimmer A D, Glinskii A B, Pinilla C, Hoffman R M, Reed J C, Glinsky G V. Increased expression of apoptosis inhibitor protein XIAP contributes to anoikis resistance of circulating human prostate cancer metastasis precursor cells. Cancer Res. 2005; 65: 2378-86.
43. Glinsky G V, Glinskii A B, Berezovskaya O, Smith B A, Jiang P, Li X M, Yang M, Hoffman R M. Dual-color-coded imaging of viable circulating prostate carcinoma cells reveals genetic exchange between tumor cells in vivo, contributing to highly metastatic phenotypes. Cell Cycle. 2006; 5: 191-7.
44. Holt, S., Glinsky, V.V., Ivanova, A.B., Glinsky, G.V. Resistance to apoptosis in human cells conferred by telomerase function and telomere stability. Molecular Carcinogenesis 1999; 25: 241-248.
45. Glinsky, G.V., Glinsky, V.V., Ivanova, A.B., Hueser, C.N. Apoptosis and metastasis: Increased apoptosis resistance of metastatic cancer cells is associated with the profound deficiency of apoptosis execution mechanisms. Cancer Letters 1997; 115: 185-193.
46. Glinsky, G.V. Apoptosis in metastatic cancer cells. Crit. Rev. Oncol/Hemat. 1997; 25: 175-186.
47. Glinsky, G V, Glinsky, V V. Apoptosis and metastasis: A superior resistance of metastatic cancer cells to programmed cell death. Cancer Letters 1996; 101: 43-51.
48. Glinsky G V. Stem cell origin of death-from-cancer phenotypes of human prostate and breast cancers. Stem Cells Reviews 2007; 3: 79-93.
49. Glinsky G V. “Stemness ” genomics law governs clinical behavior of human cancer: Implications for decision making in disease management. Journal of Clinical Oncology 2008; 26:2 846-53.
50. Glinsky G V, Berezovska O, Glinskii A. Genetic signatures of regulatory circuitry of embryonic stem cells (ESC) identify therapy-resistant phenotypes in cancer patients diagnosed with multiple types of epithelial malignancies. Cancer Research 2007; 67 (9 Supplement):1272.
51. Glinskii A, Berezovskaya O, Sidorenko A, Glinsky G. Stemness pathways define therapy-resistant phenotypes of human cancers. Clinical Cancer Research 2008; 14 (15 Supplement):B38.
52. Schwartzberg P, Colicelli J, Goff S P. Recombination between a defective retrovirus and homologous sequences in host DNA: reversion by patch repair. J Virol. 1985; 53: 719-26.
53. McClure H M. Tumors in nonhuman primates: observations during a six-year period in the Yerkes primate center colony. Am J Phys Anthropol. 1973; 38:425-429.
54. Seibold H R, Wolf R H. Neoplasms and proliferative lesions in 1065 nonhuman primate necropsies. Lab Anim Sci. 1973; 23:533-539.
55. Beniashvili D S. An overview of the world literature on spontaneous tumors in nonhuman primates. J Med Primatol. 1989; 18:423-437.
56. Scott, G.B.D. 1992. Comparative primate pathology. Oxford University Press, New York, NY.
57. Waters D J, Sakr W A, Hayden D W, Lang C M, McKinney L, Murphy G P, Radinsky R, Ramoner R, Richardson R C, Tindall D J. Workgroup 4: spontaneous prostate carcinoma in dogs and nonhuman primates. Prostate. 1998; 36: 64-67.
58. Simmons H A, Mattison J A. The incidence of spontaneous neoplasia in two populations of captive rhesus macaques (Macaca mulatta). Antioxid Redox Signal. 2011; 14: 221-7.
59. Gemmell, P., Hein, J., Katzourakis, A. Orthologous endogenous retroviruses exhibit directional selection since the chimp-human split. Retrovirology 2015; 12: 52.
60. Subramanian, R.P., Wildschutte, J.H., Russo, C., Coffin, J.M. Identification, characterization, and comparative genomic distribution of the HERV-K (HML-2) group of human endogenous retroviruses. Retrovirology 2011; 8: 90.
61. Hohn, O., Hanke, K., Bannert, N. HERV-K(HML-2), the best preserved family of HERVs: Endogenization, expression, and implications in health and disease. Front Oncol 2013; 3: 246.
62. Bhardwaj, N., Coffin, J.M. Endogenous Retroviruses and Human Cancer: Is There Anything to the Rumors? Cell Host & Microbes 2014; 15: 255-250.
63. Kent, W J. BLAT—the BLAST-like alignment tool. Genome Res. 2002; 12: 656-664.
64. Schwartz, S., Kent, W.J., Smit, A., Zhang, Z., Baertsch, R., Hardison, R.C., Haussler, D., and Miller, W. Human-mouse alignments with BLASTZ. Genome Res. 2003; 13: 103-107.
65. Tay, S.K., Blythe, J., and Lipovich, L. Global discovery of primate-specific genes in the human genome. Proc. Natl. Acad. Sci. USA 2009; 106: 12019-12024.
66. Capra, J.A., Erwin, G.D., McKinsey, G., Rubenstein, J.L., Pollard, K.S. Many human accelerated regions are developmental enhancers. Philos Trans R Soc Lond B Biol Sci. 2013; 368 (1632): 20130025.
67. Marnetto D, Molineris I, Grassi E, Provero P. Genome-wide identification and characterization of fixed human-specific regulatory regions. Am J Hum Genet 2014; 95: 39-48.
68. Gittelman R M, Hun E, Ay F, Madeoy J, Pennacchio L, Noble W S, Hawkins R D, Akey J M. 2015. Comprehensive identification and analysis of human accelerated regulatory DNA. Genome Res 2015; 25: 1245-55.
69. Guttman, M., Donaghey, J., Carey, B.W., Garber, M., Grenier, J.K., Munson, G., Young, G., Lucas, A.B., Ach, R., Bruhn, L., Yang, X., Amit, I., Meissner, A., Regev, A., Rinn, J.L., Root, D.E., and Lander, E.S. lincRNAs act in the circuitry controlling pluripotency and differentiation. Nature 2011; 477: 295-300.
70. Glinsky, G V. Rapidly evolving in humans topologically associating domains. 2015. arXiv:1507.05368 .
71. Dixon, J.R., Selvaraj, S., Yue, F., Kim, A., Li, Y., Shen, Y., Hu, M., Liu, J.S., and Ren, B. Topological domains in mammalian genomes identified by analysis of chromatin interactions. Nature 2012; 485: 376-380.
72. Dowen J.M., Fan Z.P., Hnisz D., Ren G., Abraham B.J., Zhang L.N., Weintraub A.S., Schuijers J., Lee T.I., Zhao K., Young R A. Control of cell identity genes occurs in insulated neighborhoods in mammalian chromosomes. Cell 2014; 159: 374-387.
73. Hnisz, D., Abraham, B.J., Lee, T.I., Lau, A., Saint-Andre′, V., Sigova, A.A., Hoke, H.A., and Young, R A. Super-enhancers in the control of cell identity and disease. Cell 2013; 155: 934-947.
74. Whyte, W.A., Orlando, D.A., Hnisz, D., Abraham, B.J., Lin, C.Y., Kagey, M.H., Rahl, P.B., Lee, T.I., and Young, R A. Master transcription factors and mediator establish super-enhancers at key cell identity genes. Cell 2013; 153: 307-319.
75. Meyer, L.R., Zweig, A.S., Hinrichs, A.S., Karolchik, D., Kuhn, R.M., Wong, M., Sloan, C.A., Rosenbloom, K.R., Roe, G., Rhead, B., Raney, B.J., Pohl, A., Malladi, V.S., Li, C.H., Lee, B.T., Learned, K., Kirkup, V., Hsu, F., Heitner, S., Harte, R.A., Haeussler, M., Guruvadoo, L., Goldman, M., Giardine, B.M., Fujita, P.A., Dreszer, T.R., Diekhans, M., Cline, M.S., Clawson, H., Barber, G.P., Haussler, D., and Kent, W.J. The UCSC Genome Browser database: extensions and updates 2013. Nucleic Acids Res. 2013; 41: D64-69.
76. Lister, R., Pelizzola, M., Dowen, R.H., Hawkins, R.D., Hon, G., Tonti-Filippini, J., Nery, J.R., Lee, L., Ye, Z., Ngo, Q.M., Edsall, L., Antosiewicz-Bourget, J., Stewart, R., Ruotti, V., Millar, A.H., Thomson, J.A., Ren, B., and Ecker, J R. Human DNA methylomes at base resolution show widespread epigenomic differences. Nature 2009; 462: 315-322.
77. Lister R, Mukamel E A, Nery J R, Urich M, Puddifoot C A, Johnson N D, Lucero J, Huang Y, Dwork A J, Schultz M D, Yu M, Tonti-Filippini J, Heyn H, Hu S, Wu J C, Rao A, Esteller M, He C, Haghighi F G, Sejnowski T J, Behrens M M, Ecker J R. Global epigenomic reconfiguration during mammalian brain development. Science 2013; 341: 1237905.
78. Rosenbloom, K.R., Sloan, C.A., Malladi, V.S., Dreszer, T.R., Learned, K., Kirkup, V.M., Wong, M.C., Maddren, M., Fang, R., Heitner, S.G., Lee, B.T., Barber, G.P., Harte, R.A., Diekhans, M., Long, J.C., Wilder, S.P., Zweig, A.S., Karolchik, D., Kuhn, R.M., Haussler, D., and Kent, W J. ENCODE data in the UCSC Genome Browser: year 5 update. Nucleic Acids Res 2013; 41: D56-63.
79. Li, G., Ruan, X., Auerbach, R.K., Sandhu, K.S., Zheng, M., Wang, P., Poh, H.M., Goh, Y., Lim, J., Zhang, J., Sim, H.S., Peh, S.Q., Mulawadi, F.H., Ong, C.T., Orlov, Y.L., Hong, S., Zhang, Z., Landt, S., Raha, D., Euskirchen, G., Wei, C.L., Ge, W., Wang, H., Davis, C., Fisher-Aylor, K.I., Mortazavi, A., Gerstein, M., Gingeras, T., Wold, B., Sun, Y., Fullwood, M.J., Cheung, E., Liu, E., Sung, W.K., Snyder, M., and Ruan, Y. Extensive promoter-centered chromatin interactions provide a topological basis for transcription regulation. Cell 2012; 148: 84-98.
80. Wang, J., Zhuang, J., Iyer, S., Lin, X., Whitfield, T.W., Greven, M.C., Pierce, B.G., Dong, X., Kundaje, A., Cheng, Y., Rando, O.J., Birney, E., Myers, R.M., Noble, W.S., Snyder, M., and Weng, Z. Sequence features and chromatin structure around the genomic regions bound by 119 human transcription factors. Genome Res. 2012; 22: 1798-1812.
81. Ernst, J., and Kellis, M. 2013. Interplay between chromatin state, regulator binding, and regulatory motifs in six human cell types. Genome Res. 2013; 23: 1142-1154. 82. Reich, D., Green, R.E., Kircher, M., Krause, J., Patterson, N., Durand, E.Y., Viola, B., Briggs, A.W., Stenzel, U., Johnson, P.L., Maricic, T., Good, J.M., Marques-Bonet, T., Alkan, C., Fu, Q., Mallick, S., Li, H., Meyer, M., Eichler, E.E., Stoneking, M., Richards, M., Talamo, S., Shunkov, M.V., Derevianko, A.P., Hublin, J.J., Kelso, J., Slatkin, M., Paabo, S. Genetic history of an archaic hominin group from Denisova Cave in Siberia. Nature 2010; 468: 053-1060.
83. Meyer, M., Kircher, M., Gansauge, M.T., Li, H., Racimo, F., Mallick, S., Schraiber, J.G., Jay, F., Prüfer, K., de Filippo, C., Sudmant, P.H., Alkan, C., Fu, Q., Do, R., Rohland, N., Tandon, A., Siebauer, M., Green, R.E., Bryc, K., Briggs, A.W., Stenzel, U., Dabney, J., Shendure, J., Kitzman, J., Hammer, M.F., Shunkov, M.V., Derevianko, A.P., Patterson, N., Andrés, A.M., Eichler, E.E., Slatkin, M., Reich, D., Kelso, J., Pääbo, S. A high-coverage genome sequence from an archaic Denisovan individual. Science 2012; 338: 222-226.
84. Marchler-Bauer A, Lu S, Anderson J B, Chitsaz F, Derbyshire M K, DeWeese-Scott C, Fong J H, Geer L Y, Geer R C, Gonzales N R, Gwadz M, Hurwitz D I, Jackson J D, Ke Z, Lanczycki C J, Lu F, Marchler G H, Mullokandov M, Omelchenko M V, Robertson C L, Song J S, Thanki N, Yamashita R A, Zhang D, Zhang N, Zheng C, Bryant S H. CDD: a Conserved Domain Database for the functional annotation of proteins. Nucleic Acids Res. 2011; 39: D225-9.
85. Marchler-Bauer A, Derbyshire M K, Gonzales N R, Lu S2, Chitsaz F, Geer L Y, Geer R C, He J, Gwadz M, Hurwitz D I, Lanczycki C J, Lu F, Marchler G H, Song J S, Thanki N, Wang Z, Yamashita R A, Zhang D, Zheng C, Bryant S H. CDD: NCBI's conserved domain database. Nucleic Acids Res. 2015; 43: D222-6.
86. Tavazoie, S., Hughes, J.D., Campbell, M.J., Cho, R.J., and Church, G M. 1999. Systematic determination of genetic network architecture. Nat. Genet.1999; 22: 281-285.

TABLE 1A
Enrichment analysis of LTR7/HERVH/LBP9-regulated genes in single cells from
human embryos cultured at the one- to approximately eight-cell stage.

| | | | Ratio of | | |
|---|---|---|---|---|---|
| | | Number of | HERVH/LBP9 | Fold enrichment | |
| | | HERVH/LBP9 | regulated/non- | of HERVH/LBP9 | |
| | Number of | regulated | regulated | regulated | |
| Gene category | genes | genes* | genes** | genes*** | P value**** |
| Human Embryo | 29 | 11 | 0.6 | 1.0 | 0.185 |
| Development Cluster 1 | | | | | |
| Human Embryo | 4 | 2 | 1.0 | 1.6 | 0.339 |
| Development Cluster 2 | | | | | |
| Human Embryo | 10 | 4 | 0.7 | 1.1 | 0.264 |
| Development Cluster 3 | | | | | |
| Human Embryo | 12 | 5 | 0.7 | 1.2 | 0.237 |
| Development Cluster 4 | | | | | |
| 55-gene Human Embryo | 55 | 22 | 0.7 | 1.1 | 0.160 |
| Development Signature | | | | | |
| Euploid vs Aneuploid | 22 | 12 | 1.2 | 2.0 | 0.037 |
| Embryos (p < 0.05) | | | | | |
| 12-gene Aneuploidy | 12 | 8 | 2.0 | 3.3 | 0.025 |
| Predictor | | | | | |
| Human Embryonic | 87 | 33 | 0.6 | 1.0 | NA |
| Development Associated | | | | | |
| Genes | | | | | |

Legends:
shHERVH or shLBP9, small haipin RNAs against HERVH or LBP9;
NA, not applicable;
*Number of genes with significant expression changes in both shHERVH and shLBP9 experiments;
**Ratio of HERVH/LBP9 regulated genes to genes expression of which was not significantly changed;
***Fold enrichment of HERVH/LBP9 regulated genes was calculated compared to the entire set of 87-genes associated with the human embryo development;
****P values were estimated using the hypergeometric distribution test;

TABLE 1
Distribution of conserved and human-specific regulatory sequences derived from the full-length LTR7/HERVH endogenous
human stem cell-associated retroviruses (SCARs) with distinct patterns of activation in human embryonic stem cells (hESC)

| | | Conserved in | Percent conserved | Reciprocal | Bonobo & | | | |
|---|---|---|---|---|---|---|---|---|
| Full-length | Human | non-human | in non-human | conversion | Chimpanzee | Candidate | Percent | |
| SCAR's loci | genome | primates* | primates | failure | conversion failures | HSRS** | HSRS** | P value^{#} |
| Highly active | 117 | 73 | 62.4 | 6 | 38 | 44 | 37.6 | <0.0001 |
| LTR/HERVH | | | | | | | | |
| elements | | | | | | | | |
| Moderately active | 433 | 308 | 71.1 | 25 | 100 | 125 | 28.9 | 0.0006 |
| LTR/HERVH | | | | | | | | |
| elements | | | | | | | | |
| Inactive | 672 | 539 | 80.2 | 20 | 113 | 133 | 19.8 | |
| LTR/HERVH | | | | | | | | |
| elements | | | | | | | | |
| LTR7/HERVH- | 48 | 28 | 58.3 | 5 | 15 | 20 | 41.7 | 0.0008 |
| derived IncRNA | | | | | | | | |
| expressed in | | | | | | | | |
| hESC & hiPSC | | | | | | | | |
| LTR7/HERVH- | 128 | 81 | 63.3 | 6 | 41 | 47 | 36.7 | <0.0001 |
| derived RNAs | | | | | | | | |
| most highly | | | | | | | | |
| expressed in | | | | | | | | |
| hESC | | | | | | | | |
| Full-length | 1,222*** | 920 | 75.3 | 51 | 251 | 302 | 24.7 | |
| LTR/HERVH | | | | | | | | |
| elements | | | | | | | | |

Legends:
*Sequences conserved in non-human primates were defined based on successful direct and reciprocal conversions between human, bonobo, and chimpanzee reference genome databases using the LiftOver algorithm (MinMatch threshold setting of 0.95) as described in [3];
**HSRS, human-specific regulatory sequences;
***Sequences of 1,222 full-length LTR7/HERVH were successfully converted between hg19 and hg38 database releases of the human reference genome;
^{#}Two-sided Fisher's exact test versus inactive LTR7/HERVH elements.

TABLE 2
Distribution of human-specific insertions and deletions within DNA sequences of candidate HSRS* derived from the
full-length LTR7/HERVH endogenous human SCARs^{&} with distinct patterns of activation in human embryonic stem cells.

| Genomic loci of | | | | | | | | | |
|---|---|---|---|---|---|---|---|---|---|
| endogenous human | | | | | | | | | |
| stem cell- | | | | | Percent | | Percent | | |
| associated | | Conserved in | | Human- | human- | Human- | human- | Number of loci | Percent of loci |
| retroviruses | Human | non-human | Number | specific | specific | specific | specific | with HS deletions' | with HS deletions' |
| (SCARs) | genome | primates** | of HSRS | insertions | insertions | deletions | deletions | cascade events^{#} | cascade events^{#} |
| Highly active | 117 | 73 | 44 | 35 | 79.5 | 39 | 88.6 | 26 | 59.1 |
| LTR/HERVH | | | | | | | | | |
| elements | | | | | | | | | |
| Moderately active | 433 | 308 | 125 | 99 | 79.2 | 93 | 74.4 | 62 | 49.6 |
| LTR/HERVH | | | | | | | | | |
| elements | | | | | | | | | |
| Inactive | 672 | 539 | 133 | 95 | 71.4 | 79 | 59.4 | 70 | 52.6 |
| LTR/HERVH | | | | | | | | | |
| elements | | | | | | | | | |
| LTR7/HERVH- | 48 | 28 | 20 | 15 | 75.0 | 16 | 80.0 | 13 | 65.0 |
| derived IncRNA*** | | | | | | | | | |
| expressed in hESC | | | | | | | | | |
| & hiPSC**** | | | | | | | | | |

Legends:
*HSRS, human-specific regulatory sequences;
^{&}SCARs, stem cell-associated retroviruses;
**Sequences conserved in non-human primates were defined based on successful direct and reciprocal conversions between human, bonobo, and chimpanzee reference genome databases using the LiftOver algorithm (MinMatch setting of 0.95) as described in [3];
***IncRNAs, long noncoding RNAs;
****hiPSC, human induced pluripotent stem cells;
^{#}Number (percent) of loci with at least 2 distinct events of human-specific (HS) DNA deletions compared to genomes of at least 2 different species of non-human primates selected from the group comprising of chimpanzee, bonobo, gorilla, orangutan, and gibbon; hESC, human embryonic stem cells.

TABLE 3
Identification of candidate human-specific virus/host chimeric transcripts associated with naïve-state hESCs.

| | | | | | | |
|---|---|---|---|---|---|---|
| Number of | Bonobo | Chimp | Conserved in non- | Percent conserved | Candidate human- | Percent |
| chimeric | conversion | conversion | human primates | in non-human | specific regulatory | human- |
| transcripts* | failures | failures | chimeric transcripts** | primates | sequences*** | specific |

3.1. Distribution patterns of virus/host chimeric transcripts detected in ELF1 naïve vs. primed hESC cells.

| | | | | | | |
|---|---|---|---|---|---|---|
| 38 | 10 | 7 | 33 | 86.8 | 5 | 13.2 |
| 36 | 13 | 9 | 29 | 80.6 | 7 | 19.4 |
| 37 | 8 | 11 | 33 | 89.2 | 4 | 10.8 |

3.2. All ERV1/host chimeric transcripts reported by Grow et al. (2015).

| | | | | | | |
|---|---|---|---|---|---|---|
| 364 | 107 | 106 | 300 | 82.4 | 64 | 17.6 |

3.3. Genomic regions consistently generating human-specific virus/host chimeric transcripts in naïve-state hESCs.

| Genomic | Genomic | Number of | | Genomic coordinates | Genomic | Sequences | |
|---|---|---|---|---|---|---|---|
| coordinates of the | size of the | chimeric | Repeats' sequence structure of | of the human-specific | size of the | of human | Comments on human-specific |
| region (hg38) | region | transcripts | human-specific insert | insert (hg38) | insert | genes | regions |
| chr11: 62357061-62381889 | 24,828 bp. | 4 | Zaphod/AluSx/Zaphod/Zaphod/ | chr11: 62,359, 700-62,364,100 | 4,401 bp. | ASRGL1 | Human specific region created |
| | | | AluJo/AluSx4/Zaphod/A- | | | intron | by DNA and SINE (Alu) |
| | | | rich/(AC)n/Zaphod/ | | | | repeats |
| | | | AluY/Zaphod/AluSx3 | | | | |
| chr5: 1579414-1589336 | 9,922 bp. | 8 | HERVK9-int/MER9a3/SVA_D | chr5: 1,581,000-1,587,500 | 6,501 bp. | SDHAP3 | Created by HERVK9- |
| | | | | | | pseudogene | int/MER9a4 and SVA_D |
| | | | | | | | repeats |
| chr13: 45370126-45383162 | 13,036 bp. | 28 | HERVE-int/HERVE-int/ | chr13: 45376607-45383238 | 6,632 bp. | TPT1 | Sub-regions created by six |
| | | | HERVE-int/HERVE-int/ | | | antisense | HERVE-int repeats and |
| | | | HERVE-int/HERVE-int | | | RNA 1 | multiple deletions of non- |
| | | | | | | | human primates' sequences |
| chr5: 147870455-147881521 | 11,067 bp. | 1 | HERVH-int/LTR7/MER61- | chr5: 147864645-147874526 | 9,882 bp. | SCGB3A2 | Created by two HERVH/LTR7 |
| | | | int/LTR8/LTR7/HERVH- | | | exon 1 & | integration sites |
| | | | int/LTR7/LTR8/MER74a | | | intron 1 | |
| chrX: 53576971-53580926 | 3,956 bp. | 1 | SVA_E | chrX: 53577490-53579966 | 2,477 bp. | HUWE1 | Human-specific region |
| | | | | | | intron | created by SVA_E repeats |
| chr2: 187555926-187566148 | 10,223 bp. | 3 | SVA_D/SVA_D/(AAAAT)n/LTR7/ | chr2: 187555926-187557937 | 2,012 bp. | Intergenic | Sub-region created by two |
| | | | HERVH-int/HERVH-int/LTR7 | | | near TFPI | SVA_D and seven (AAAAT)n |
| | | | | | | gene | repeats |
| chr3: 109300370-109308123 | 7,754 bp | 2 | Several distinct structures | Several distinct | Several | DPPA2 | Several distinct human- |
| | | | | genomic locations | distinct | intron/exon/ | specific sites compared to |
| | | | | | sites | intron | other primates |
| chrY: 278899-284215 | 5,317 bp. | 2 | LTR7C/MER4B/AluSx/MER4B/ | Two distinct human- | | PLCXD1 | Distinct patterns of human- |
| chrX: 278899-284215 | | | AluSx & | specific genomic sites | | gene: intron | specific sequences with |
| | | | AluSx/(TCTAA)n/AluSq2/ | on chrY & chX | | 1/exon | intermitted homology regions |
| | | | AluSq2/MER67C/(TA)n/ | | | 2/intron 2 | on chrX and chrY compared |
| | | | (TG)n/LTR9B/AluSp/ | | | sequence | to other primates |
| | | | LTR9B/LT9B/AluSq | | | | |

Legends:
*Genomic identities of chimeric transcripts from 3 biological replicates [20];
**Sequences conserved in non-human primates were defined based on successful conversions between human, bonobo, and chimpanzee reference genome databases using the LiftOver algorithm (MinMatch setting of 0.95) as described in [3];
***Candidate human-specific regulatory sequences were defined based on conversion failures from the human genome to the genomes of both bonobo and chimpanzee. In bold, genomic coordinates of the regions generating in the hESC virus/host chimeric transcripts encoding GVQW conserved protein domains.

TABLE 4
Data for FIGS. 1A-1K

| N | FIG. 1A | | P value | Data set | N | FIG. 1C | | P value | Data set |
|---|---|---|---|---|---|---|---|---|---|
| 5,158 | PLCXD1 | Gene expression | 1.78E−09 | TCGA PANCAN12 | 5,158 | CCL26 | Gene expression | 0.007 | TCGA PANCAN12 |
| 5,158 | ZNF443 | Gene expression | 0.00E+00 | TCGA PANCAN12 | 5,158 | PLCXD1 | Gene expression | 1.78E−09 | TCGA PANCAN12 |
| 5,158 | LRBA | Gene expression | 0.00E+00 | TCGA PANCAN12 | 5,158 | ZNF443 | Gene expression | 0.00E+00 | TCGA PANCAN12 |
| 5,158 | TPT1 | Gene expression | 5.27E−06 | TCGA PANCAN12 | 5,158 | LRBA | Gene expression | 0.00E+00 | TCGA PANCAN12 |
| 5,158 | ABHD12B | Gene expression | 5.26E−05 | TCGA PANCAN12 | | | | | |
| 5,158 | LIN7A | Gene expression | 0.00031 | TCGA PANCAN12 | | | | | |

| N | FIG. 1B | | P value | Data set | N | FIG. 1D | | P value | Data set |
|---|---|---|---|---|---|---|---|---|---|
| 568 | PLCXD1 | Exon expression | 0.0052 | TCGA Prostate | 5,158 | ZNF443 | Gene copy number | 4.66E−15 | TCGA PANCAN12 |
| | | | | cancer | | | | | |
| 1,241 | RHOT1 | Gene expression | 0.026 | TCGA Breast | 5,158 | ZNF587 | Gene copy number | 3.86E−09 | TCGA PANCAN12 |
| | | | | cancer | | | | | |
| 1,241 | RHOT1 | Exon expression | 0.012 | TCGA Breast | 5,158 | ZNF814 | Gene copy number | 3.72E−09 | TCGA PANCAN12 |
| | | | | cancer | | | | | |
| 187 | TPT1 | Gene expression | 0.037 | TCGA Rectal | 5,158 | CCL26 | Gene copy number | 0.00E+00 | TCGA PANCAN12 |
| | | | | cancer | | | | | |
| 187 | HUWE1 | Gene expression | 0.041 | TCGA Rectal | | | | | |
| | | | | cancer | | | | | |

TABLE 5
Data for FIG. 4A-4D

| | | | P value | P value | N | | | | | | | | |
|---|---|---|---|---|---|---|---|---|---|---|---|---|---|
| | | | TCGA Breast | PTCGA Pan-Cancer | | | | | | | | | |
| N | FIG. 4B | | cancer | 12K | | N | FIG. 4C | | P value | Data set | N | P value | Data set |
| 1,241 | ZNF546 | Gene | 0.014 | 0.00E+00 | 12,093 | 550 | ZNF385A | Exon | 0.02 | TCGA | 12,093 | | PTCGA |
| | | expression | | | | | | expression | | Colon | | | Pan- |
| | | | | | | | | | | cancer | | | Cancer |
| | | | | | | | | | | | | | 12K |
| 1,241 | ZNF763 | Gene | 0.042 | 0.00E+00 | 12,093 | 550 | ZNF385A | Gene | 0.0092 | TCGA | 12,093 | 0.013 | PTCGA |
| | | expression | | | | | | expression | | Colon | | | Pan- |
| | | | | | | | | | | cancer | | | Cancer |
| | | | | | | | | | | | | | 12K |
| 1,241 | ZNF283 | Gene | 0.045 | 0.033 | 12,093 | 187 | ZNF283 | Exon | 2.66E−05 | TCGA | 12,093 | | PTCGA |
| | | expression | | | | | | expression | | Rectal | | | Pan- |
| | | | | | | | | | | cancer | | | Cancer |
| | | | | | | | | | | | | | 12K |
| 1,241 | AEBP2 | Gene | 0.0009 | 0.11 | 12,093 | 187 | ZNF283 | Gene | 0.011 | TCGA | 12,093 | 0.033 | PTCGA |
| | | expression | | | | | | expression | | Rectal | | | Pan- |
| | | | | | | | | | | cancer | | | Cancer |
| | | | | | | | | | | | | | 12K |
| 1,241 | ZNF83 | Gene | 0.071 | 0.00E+00 | 12,093 | 1,241 | ZNF546 | Gene | 0.015 | TCGA | 12,093 | | PTCGA |
| | | expression | | | | | | expression | | Breast | | | Pan- |
| | | | | | | | | | | cancer | | | Cancer |
| | | | | | | | | | | | | | 12K |
| 1,241 | ZNF611 | Gene | 0.04 | 4.15E−07 | 12,093 | 196 | ZNF546 | Gene | 0.044 | TCGA | 12,093 | 0.00E+00 | PTCGA |
| | | expression | | | | | | expression | | Pancre- | | | Pan- |
| | | | | | | | | | | atic | | | Cancer |
| | | | | | | | | | | cancer | | | 12K |

| | | | P value | | | | | | | | | | |
|---|---|---|---|---|---|---|---|---|---|---|---|---|---|
| | | | TCGA | P value | N | | | | | | | | |
| | | | Prostate | PTCGA Pan-Cancer | | | | | | | | | |
| N | FIG. 4A | | cancer | 12K | | N | FIG. 4D | | P value | Data set | N | P value | Data set |
| 568 | HKR1 | Gene/exon | 0.00046 | 0.00E+00 | 12,093 | 5,158 | ZNF546 | Gene | 3.12E−11 | TCGA | 12,093 | 0.00E+00 | PTCGA |
| | | expression | | | | | | copy | | PANCAN12 | | | Pan- |
| | | | | | | | | number | | | | | Cancer |
| | | | | | | | | | | | | | 12K |
| 568 | ZNF546 | Gene/exon | 0.57 | 0.00E+00 | 12,093 | 5,158 | ZNF763 | Gene | 1.33E−15 | TCGA | 12,093 | 0.00E+00 | PTCGA |
| | | expression | | | | | | copy | | PANCAN12 | | | Pan- |
| | | | | | | | | number | | | | | Cancer |
| | | | | | | | | | | | | | 12K |
| 568 | ZNF611 | Gene/exon | 0.76 | 4.15E−07 | 12,093 | 5,158 | ZNF283 | Gene | 4.30E−11 | TCGA | 12,093 | 0.00E+00 | PTCGA |
| | | expression | | | | | | copy | | PANCAN12 | | | Pan- |
| | | | | | | | | number | | | | | Cancer |
| | | | | | | | | | | | | | 12K |
| 568 | ZNF283 | Gene/exon | 0.24 | 0.033 | 12,093 | 5,158 | HKR1 | Gene | 5.18E−10 | TCGA | 12,093 | 0.00E+00 | PTCGA |
| | | expression | | | | | | copy | | PANCAN12 | | | Pan- |
| | | | | | | | | number | | | | | Cancer |
| | | | | | | | | | | | | | 12K |
| 568 | ZNF28 | Gene/exon | 0.15 | 4.42E−06 | 12,093 | | | | | | | | |
| | | expression | | | | | | | | | | | |
| 568 | ZNF385A | Gene/exon | 0.19 | 0.013 | 12,093 | | | | | | | | |
| | | expression | | | | | | | | | | | |
| 568 | PLCXD1 | Gene/exon | 0.0052 | 0.00E+00 | 12,093 | | | | | | | | |
| | | expression | | | | | | | P value | Data set | N | P value | Data set |
| | | | | | | | ZNF611 | Gene | 1.13E−10 | TCGA | 12,093 | 0.00E+00 | PTCGA |
| | | | | | | | | copy | | PANCAN12 | | | Pan- |
| | | | | | | | | number | | | | | Cancer |
| | | | | | | | | | | | | | 12K |
| | | | | | | | ZNF385A | Gene | 1.41E−05 | TCGA | 12,093 | 0.00E+00 | PTCGA |
| | | | | | | | | copy | | PANCAN12 | | | Pan- |
| | | | | | | | | number | | | | | Cancer |
| | | | | | | | | | | | | | 12K |
| | | | | | | | ZNF28 | Gene | 1.13E−10 | TCGA | 12,093 | 0.00E+00 | PTCGA |
| | | | | | | | | copy | | PANCAN12 | | | Pan- |
| | | | | | | | | number | | | | | Cancer |
| | | | | | | | | | | | | | 12K |
| | | | | | | | AEBP2 | Gene | 3.25E−09 | TCGA | 12,093 | 7.30E−13 | PTCGA |
| | | | | | | | | copy | | PANCAN12 | | | Pan- |
| | | | | | | | | number | | | | | Cancer |
| | | | | | | | | | | | | | 12K |
| | | | | | | | ZNF83 | Gene | 1.95E−10 | TCGA | 12,093 | 0.00E+00 | PTCGA |
| | | | | | | | | copy | | PANCAN12 | | | Pan- |
| | | | | | | | | number | | | | | Cancer |
| | | | | | | | | | | | | | 12K |

| | SCARs network | | | | | | |
|---|---|---|---|---|---|---|---|
| | ZNFs | | | | | | |
| chr19: | ZNF443 | Gene | 5.55E−16 | TCGA | 12,093 | 0.00E+00 | PTCGA |
| 12,429, | | copy | | PANCAN12 | | | Pan- |
| 707-12, | | number | | | | | Cancer |
| 441, | | | | | | | 12K |
| 112 | | | | | | | |
| chr19: | ZNF587 | Gene | 8.12E−10 | TCGA | 12,093 | 0.00E+00 | PTCGA |
| 57,849, | | copy | | PANCAN12 | | | Pan- |
| 857-57, | | number | | | | | Cancer |
| 865, | | | | | | | 12K |
| 112 | | | | | | | |
| chr19: | ZNF814 | Gene | 7.96E−10 | TCGA | 12,093 | 0.00E+00 | PTCGA |
| 57,864, | | copy | | PANCAN12 | | | Pan- |
| 765-57, | | number | | | | | Cancer |
| 888, | | | | | | | 12K |
| 780 | | | | | | | |

TABLE 6
Data for FIGS. 5A-5D

| | | | | | | | | | | Pradigm |
|---|---|---|---|---|---|---|---|---|---|---|
| | | | | | | | | | | IPLs |
| | GVQW Zinc | | | | | Data | | | | (Five3 |
| | Finger Proteins | | | | P value | set | | Order in the FIG. 5 | | Genomics) |
| chr11: | ZNF195 | Zinc | Gene copy | 12,093 | 0.00E+00 | PTCGA Pan- | | ZNF763 | 0.00E+00 | |
| 3,357,927-3,379,145 | | finger | number | | | Cancer 12K | | | | |
| | | protein | changes | | | | | | | |
| chr12: | AEBP2 | Zinc | Gene copy | 12,093 | 7.30E−13 | PTCGA Pan- | | ZNF283 | 0.00E+00 | |
| 19,439,674-19,522,239 | | finger | number | | | Cancer 12K | | | | |
| | | protein | changes | | | | | | | |
| | | AEBP2 | | | | | | | | |
| chr12: | ZNF385A | Zinc | Gene copy | 12,093 | 0.00E+00 | PTCGA Pan- | | HKR1 | 0.00E+00 | |
| 54,369,140-54,391,298 | | finger | number | | | Cancer 12K | | | | |
| | | protein | changes | | | | | | | |
| | | 385A | | | | | | | | |
| chr19: | ZNF763 | Zinc | Gene copy | 12,093 | 0.00E+00 | PTCGA Pan- | | ZNF611 | 0.00E+00 | |
| 11,965,054-11,980,381 | | finger | number | | | Cancer 12K | | | | |
| | | protein | | | | | | | | |
| | | 763 | changes | | | | | | | |
| chr19: | ZNF20 | Zinc | Gene copy | 12,093 | 0.00E+00 | PTCGA Pan- | | ZNF385A | 0.00E+00 | 5.36E−12 |
| 12,131,350-12,140,407 | | finger | number | | | Cancer 12K | | | | |
| | | protein | changes | | | | | | | |
| chr19: | ZNF100 | Zinc | Gene copy | 12,093 | 0.00E+00 | PTCGA Pan- | | ZNF28 | 0.00E+00 | 1.15E−09 |
| 21,726,529-21,767,498 | | finger | number | | | Cancer 12K | | | | |
| | | protein | changes | | | | | | | |
| chr19: | ZNF675 | Zinc | Gene copy | 12,093 | 0.00E+00 | PTCGA Pan- | | AEBP2 | 7.30E−13 | |
| 23,652,801-23,687,202 | | finger | number | | | Cancer 12K | | | | |
| | | protein | changes | | | | | | | |
| chr19: | ZNF461 | Zinc | Gene copy | 12,093 | 0.00E+00 | PTCGA Pan- | | ZNF83 | 0.00E+00 | |
| 36,637,989-36,666,837 | | finger | number | | | Cancer 12K | | | | |
| | | protein | changes | | | | | | | |
| chr19: | ZNF585B | Zinc | Gene copy | 12,093 | 0.00E+00 | PTCGA Pan- | | ZNF546 | 0.00E+00 | 0.015 |
| 37,181,579-37,210,549 | | finger | number | | | Cancer 12K | | | | |
| | | protein | changes | | | | | | | |
| | | 585B | | | | | | | | |
| chr19: | HKR1 | Zinc | Gene copy | 12,093 | 0.00E+00 | PTCGA Pan- | | ZNF816 | 0.00E+00 | |
| 37,317,911-37,364,446 | | finger | number | | | Cancer 12K | | | | |
| | | protein | changes | | | | | | | |
| | | HKR1 | | | | | | | | |
| chr19: | ZNF527 | Zinc | Gene copy | 12,093 | 0.00E+00 | PTCGA Pan- | | ZNF585B | 0.00E+00 | |
| 37,371,161-37,390,770 | | finger | number | | | Cancer 12K | | | | |
| | | protein | changes | | | | | | | |
| chr19: | ZNF546 | Zinc | Gene copy | 12,093 | 0.00E+00 | PTCGA Pan- | | ZNF20 | 0.00E+00 | 2.14E−10 |
| 39,997,076-40,021,041 | | finger | number | | | Cancer 12K | | | | |
| | | protein | changes | | | | | | | |
| chr19: | ZNF283 | Zinc | Gene copy | 12,093 | 0.00E+00 | PTCGA Pan- | | ZNF100 | 0.00E+00 | 4.69E−05 |
| 43,827,292-43,852,017 | | finger | number | | | Cancer 12K | | | | |
| | | protein | changes | | | | | | | |
| | | 283 | | | | | | | | |
| chr19: | ZNF880 | Zinc | Gene copy | 12,093 | 0.00E+00 | PTCGA Pan- | | ZNF461 | 0.00E+00 | |
| 52,369,951-52,385,795 | | finger | number | | | Cancer 12K | | | | |
| | | protein | changes | | | | | | | |
| chr19: | ZNF83 | Zinc | Gene copy | 12,093 | 0.00E+00 | PTCGA Pan- | | ZNF468 | 0.00E+00 | 9.55E−15 |
| 52,612,367-52,638,391 | | finger | number | | | Cancer 12K | | | | |
| | | protein | changes | | | | | | | |
| chr19: | ZNF611 | Zinc | Gene copy | 12,093 | 0.00E+00 | PTCGA Pan- | | ZNF527 | 0.00E+00 | |
| 52,702,813-52,735,054 | | finger | number | | | Cancer 12K | | | | |
| | | protein | changes | | | | | | | |
| chr19: | ZNF28 | Zinc | Gene copy | 12,093 | 0.00E+00 | PTCGA Pan- | | ZNF675 | 0.00E+00 | |
| 52,797,409-52,821,632 | | finger | number | | | Cancer 12K | | | | |
| | | protein | changes | | | | | | | |
| chr19: | ZNF468 | Zinc | Gene copy | 12,093 | 0.00E+00 | PTCGA Pan- | | ZNF880 | 0.00E+00 | |
| 52,838,008-52,857,619 | | finger | number | | | Cancer 12K | | | | |
| | | protein | changes | | | | | | | |
| chr19: | ZNF816 | Zinc | Gene copy | 12,093 | 0.00E+00 | PTCGA Pan- | | ZNF169 | 0.00E+00 | 4.56E−12 |
| 52,949,381-52,962,911 | | finger | number | | | Cancer 12K | | | | |
| | | protein | changes | | | | | | | |
| | | 816 | | | | | | | | |
| chr7: | ZNF212 | Zinc | Gene copy | 12,093 | 0.00E+00 | PTCGA Pan- | | ZNF195 | 0.00E+00 | 1.73E−05 |
| 149,239,651-149,255,609 | | finger | number | | | Cancer 12K | | | | |
| | | protein | changes | | | | | | | |
| chr9: | ZNF169 | Zinc | Gene copy | 12,093 | 0.00E+00 | PTCGA Pan- | | ZNF212 | 0.00E+00 | 1.32E−11 |
| 94,259,311-94,301,454 | | finger | number | | | Cancer 12K | | | | |
| | | protein | changes | | | | | | | |
| | | | | | | | SCARs | ZNF443 | 0.00E+00 | 0.00E+00 |
| | | | | | | | network genes | (ZK1) | | |
| | | | | | | | SCARs | ZNF587 | 0.00E+00 | |
| | | | | | | | network genes | | | |
| | | | | | | | SCARs | ZNF814 | 0.00E+00 | |
| | | | | | | | network genes | | | |

TABLE 7
Data for FIGS. 6A and 6B

| Gene | SNMs p value | Xena-1 |
|---|---|---|
| TP53 | 0.00E+00 | |
| PCDH15 | 2.77E−05 | |
| DMD | 0.031 | |
| NF1 | 3.93E−06 | |
| NOTCH1 | 0.016 | |
| EGFR | 0.00E+00 | |
| MALAT1 | 0.00043 | |
| RB1 | 0.00059 | |
| LPHN3 | 0.0094 | |
| KDM6A | 9.93E−05 | |
| TLR4 | 0.031 | |
| KEAP1 | 0.00011 | |
| SMAD4 | 2.58E−08 | |
| PRX | 0.01 | |
| EPHA7 | 2.53E−05 | |
| IDH1 | 0.0015 | |
| KIAA1244 | 0.0064 | |
| STK11 | 0.00011 | |
| DAB2IP | 4.21E−05 | |
| PTPN11 | 0.00023 | |
| ELF3 | 0.02 | |
| VEZF1 | 0.019 | |
| GLUD2 | 0.024 | |
| ZNF28 | 0.012 | |
| DPPA2 | 0.032 | |
| CHST6 | 0.039 | |
| FEZ2 | 0.014 | |

TABLE 8
Data for FIGS. 7A-7D

| | Gene-level copy | TCGA |
|---|---|---|
| Gene | numbers p value | Pan-Cancer 12K |
| KLF4 | 0.00E+00 | |
| LBP9 (TFCP2L1) | 0.00E+00 | |
| NANOG | 1.26E−10 | |
| POU5F1 | 0.00E+00 | |
| TP53 | 2.50E−04 | |
| PCDH15 | 0.00E+00 | |
| DMD | 0.00E+00 | |
| NF1 | 0.00E+00 | |
| NOTCH1 | 0.00E+00 | |
| EGFR | 0.00E+00 | |
| MALAT1 | 0.00E+00 | |
| RB1 | 3.29E−08 | |
| LPHN3 | 0.00E+00 | |
| KDM6A | 4.42E−13 | |
| TLR4 | 0.00E+00 | |
| KEAP1 | 0.00E+00 | |
| SMAD4 | 0.00E+00 | |
| PRX | 0.00E+00 | |
| EPHA7 | 1.91E−13 | |
| IDH1 | 1.78E−15 | |
| KIAA1244 | 0.00E+00 | |
| STK11 | 0.00E+00 | |
| DAB2IP | 0.00E+00 | |
| PTPN11 | 3.66E−15 | |
| VEZF1 | 2.56E−13 | |
| GLUD2 | 3.79E−08 | |
| ZNF28 | 0.00E+00 | |
| DPPA2 | 3.35E−09 | |
| CHST6 | 3.05E−08 | |
| FEZ2 | 1.24E−13 | |
| ADARB2 | 0.00E+00 | |
| CYP19A1 | 0.00E+00 | |
| LDB2 | 0.00E+00 | |
| BMI1 | 0.00E+00 | |
| EZH2 | 0.00E+00 | |

TABLE 9
Data for FIGS. 8A and 8B (Proteins P value)

| | | | PANCAN12 | |
|---|---|---|---|---|
| | | | protein expression | |
| gene | | | pvalue | |
| BCL2 | BCL2 | Protein | 0.00E+00 | 60.5263 |
| | | expression | | |
| INPP4B | INPP4B | Protein | 2.81E−09 | |
| | | expression | | |
| XRCC1 | XRCC1 | Protein | 3.66E−09 | |
| | | expression | | |
| SRC | SRC | Protein | 2.80E−08 | |
| | | expression | | |
| DVL3 | DVL3 | Protein | 7.19E−08 | |
| | | expression | | |
| IGFBP2 | IGFBP2 | Protein | 1.51E−07 | |
| | | expression | | |
| SHC1 | SHCPY317 | Protein | 2.58E−06 | |
| | | expression | | |
| LCK | LCK | Protein | 5.55E−06 | |
| | | expression | | |
| PCNA | PCNA | Protein | 2.33E−05 | |
| | | expression | | |
| ASNS | ASNS | Protein | 2.38E−05 | |
| | | expression | | |
| FN1 | FIBRONECTIN | Protein | 2.52E−05 | |
| | | expression | | |
| GAB2 | GAB2 | Protein | 4.11E−05 | |
| | | expression | | |
| MYC | CMYC | Protein | 5.92E−05 | |
| | | expression | | |
| SMAD4 | SMAD4 | Protein | 0.0014 | |
| | | expression | | |
| CCNE1 | CYCLINE1 | Protein | 0.0018 | |
| | | expression | | |
| SMAD1 | SMAD1 | Protein | 0.003 | |
| | | expression | | |
| EEF2K | EEF2K | Protein | 0.0037 | |
| | | expression | | |
| CCND1 | CYCLIND1 | Protein | 0.0038 | |
| | | expression | | |
| NOTCH1 | NOTCH1 | Protein | 0.0081 | |
| | | expression | | |
| TP53 | P53 | Protein | 0.013 | |
| | | expression | | |
| CAV1 | CAVEOLIN1 | Protein | 0.028 | |
| | | expression | | |
| BID | BID | Protein | 0.03 | |
| | | expression | | |
| CTNNB1 | BETACATENIN | Protein | 0.046 | |
| | | expression | | |
| EIF4E | EIF4E | Protein | 0.052 | |
| | | expression | | |
| YAP1 | YAP | Protein | 0.054 | |
| | | expression | | |
| RAD51C | RAD51 | Protein | 0.059 | |
| | | expression | | |
| EEF2 | EEF2 | Protein | 0.13 | |
| | | expression | | |
| BAX | BAX | Protein | 0.21 | |
| | | expression | | |
| SYK | SYK | Protein | 0.21 | |
| | | expression | | |
| BAK1 | BAK1 | Protein | 0.32 | |
| | | expression | | |
| MET | CMETPY1235 | Protein | 0.39 | |
| | | expression | | |
| STMN1 | STATHMIN | Protein | 0.39 | |
| | | expression | | |
| STAT3 | STAT3PY705 | Protein | 0.41 | |
| | | expression | | |
| ATM | ATM | Protein | 0.53 | |
| | | expression | | |
| SMAD3 | SMAD3 | Protein | 0.55 | |
| | | expression | | |
| AKT1 | AKT1 | Protein | 0.72 | |
| | | expression | | |
| FOXO3 | FOXO3A | Protein | 0.83 | |
| | | expression | | |
| IRS1 | IRS1 | Protein | 0.99 | |
| | | expression | | |

Tables 10-14 (Data Set S2) contain descriptions of human-specific SCARs loci defined based on the direct and reciprocal sequence alignment conversion failures during the comparisons of the human genome sequences to the sequences of the genomes of 17 the primates, including genomes of Chimpanzee, Bonobo, Gorilla, Orangutan, Gibbon, and Rhesus. Tables 10-X also denote for each SCARs loci the size of human-specific deletions of ancestral DNA defined by the sequence alignments to the genomes of 17 primates.

TABLE 10
251b.c.failures (Section A)

| 1. | | | | | | | | | | | |
|---|---|---|---|---|---|---|---|---|---|---|---|
| 2. | GENE | hg38 | Bonobo | Chimp | Expression | HUMAN_SPECIC | HUMAN_SPECIC | High | HUMAN_SPECIC INTEGRATION SITE | | |
| | | | LiftOver | LiftOver | type in | INSERTIONS | INTEGRATION SITE | Confi- | | | |
| | | | | | hESC | | | dence | | | |
| 3. | TECPR2 | chr14 | #Deleted | #Deleted | highly | YES | YES | YES | | | |
| | | 102410503 | in new | in new | active | | | | | | |
| | | 102411706 | | | | | | | | | |
| 4. | | chr19 | #Deleted | #Deleted | highly | Chimp | | | | | |
| | | 36155474 | in new | in new | active | | | | | | |
| | | 36161023 | | | | | | | | | |
| 5. | | chr1 81245282 | #Partially | #Partially | highly | YES | Bonobo closest alignment | | | | |
| | | 81251207 | deleted in | deleted in | active | | | | | | |
| | | | new | new | | | | | | | |
| 6. | LINC01356 | chr1 | #Partially | #Partially | highly | YES | chr1: | YES | HERVH/ | chr1: | chr1: |
| | | 112809666 | deleted in | deleted in | active | | 112,821,143-112,826,054 | | AluY/ | 112821143-112822269 | 112823542-112825658 |
| | | 112826054 | new | new | | | 4,912 bp | | HERVH/ | | |
| | | | | | | | | | LTR7 | | |

| | | | | | | |
|---|---|---|---|---|---|---|
| 7. | chr1 | #Partially | #Partially | highly | YES | Probable (gorilla) |
| | 212910007 | deleted in | deleted in | active | | |
| | 212914681 | new | new | | | |

| 8. | chr2 7872705 | #Partially | #Partially | highly | YES | Probable: large deletions in chimp; bonobo; gorilla |
|---|---|---|---|---|---|---|
| | 7878891 | deleted in | deleted in | active | | |
| | | new | new | | | |
| 9. | chr2 64252413 | #Partially | #Partially | highly | YES | Bonobo closest alignment |
| | 64257646 | deleted in | deleted in | active | | |
| | | new | new | | | |

| 10. | LRRTM4 | chr2 77088246 | #Partially | #Partially | highly | YES | YES | | |
|---|---|---|---|---|---|---|---|---|---|
| | | 77094030 | deleted in | deleted in | active | | | | |
| | | | new | new | | | | | |
| 11. | | chr2 | #Partially | #Partially | highly | YES | | | |
| | | 209299312 | deleted in | deleted in | active | | | | |
| | | 209304932 | new | new | | | | | |
| 12. | LPHN3 | chr4 61764217 | #Partially | #Partially | highly | YES | YES | YES | chr4: 61,757,766-61,771,477 13,712 bp. |
| | | 61770025 | deleted in | deleted in | active | | | | |
| | | | new | new | | | | | |

| | | | | | | | |
|---|---|---|---|---|---|---|---|
| 13. | LOC101929194 | chr4 92271491 | #Partially | #Partially | highly | YES | Bonobo closest alignment |
| | | 92277648 | deleted in | deleted in | active | | |
| | | | new | new | | | |
| 14. | C4orf51 | chr4 | #Partially | #Partially | highly | YES | |
| | | 145698822 | deleted in | deleted in | active | | |
| | | 145703503 | new | new | | | |
| 15. | | chr5 | #Partially | #Partially | highly | YES | |
| | | 120697545 | deleted in | deleted in | active | | |
| | | 120703411 | new | new | | | |

| 16. | | chr5 | #Partially | #Partially | highly | YES | 2 adjacent LTR7/HERVH; one human-specific | | |
|---|---|---|---|---|---|---|---|---|---|
| | | 147860285 | deleted in | deleted in | active | | | | |
| | | 147874526 | new | new | | | | | |
| 17. | | chr6 | #Partially | #Partially | highly | YES | | | |
| | | 114422438 | deleted in | deleted in | active | | | | |
| | | 114428297 | new | new | | | | | |
| 18. | | chr6 | #Partially | #Partially | highly | YES | | | |
| | | 142015665 | deleted in | deleted in | active | | | | |
| | | 142021782 | new | new | | | | | |
| 19. | SEMA3E | chr7 83459667 | #Partially | #Partially | highly | Chimp | | | |
| | | 83465383 | deleted in | deleted in | active | | | | |
| | | | new | new | | | | | |
| 20. | | chr9 12948344 | #Partially | #Partially | highly | YES | | | |
| | | 12954128 | deleted in | deleted in | active | | | | |
| | | | new | new | | | | | |
| 21. | | chr9 87410693 | #Partially | #Partially | highly | YES | YES | YES | chr9: 87409190-87418209 9,020 bp |
| | | 87416706 | deleted in | deleted in | active | | | | |
| | | | new | new | | | | | |
| 22. | | chr9 97214493 | #Partially | #Partially | highly | YES | | | |
| | | 97220014 | deleted in | deleted in | active | | | | |
| | | | new | new | | | | | |
| 23. | | chr9 | #Partially | #Partially | highly | YES | YES | | |
| | | 115473180 | deleted in | deleted in | active | | | | |
| | | 115478918 | new | new | | | | | |
| 24. | | chr10 | #Partially | #Partially | highly | YES | | | |
| | | 90081017 | deleted in | deleted in | active | | | | |
| | | 90086792 | new | new | | | | | |
| 25. | BDNF- | chr11 | #Partially | #Partially | highly | YES | | | |
| | AS; | 27629071 | deleted in | deleted in | active | | | | |
| | LINC0678 | 27634926 | new | new | | | | | |
| 26. | AP002954.4 | chr11 | #Partially | #Partially | highly | YES | | | |
| | | 118717033 | deleted in | deleted in | active | | | | |
| | | 118731855 | new | new | | | | | |
| 27. | | chr12 | #Partially | #Partially | highly | YES | | | |
| | | 14705420 | deleted in | deleted in | active | | | | |
| | | 14710640 | new | new | | | | | |
| 28. | | chr12 | #Partially | #Partially | highly | YES | | | |
| | | 59323187 | deleted in | deleted in | active | | | | |
| | | 59328986 | new | new | | | | | |
| 29. | LINC00371 | chr13 | #Partially | #Partially | highly | YES | | | |
| | | 51169865 | deleted in | deleted in | active | | | | |
| | | 51175006 | new | new | | | | | |
| 30. | | chr14 | #Partially | #Partially | highly | YES | | | |
| | | 38190637 | deleted in | deleted in | active | | | | |
| | | 38196525 | new | new | | | | | |
| 31. | MDGA2 | chr14 | #Partially | #Partially | highly | YES | | | |
| | | 47104196 | deleted in | deleted in | active | | | | |
| | | 47108765 | new | new | | | | | |
| 32. | | chr16 | #Partially | #Partially | highly | YES | | | |
| | | 13352582 | deleted in | deleted in | active | | | | |
| | | 13358061 | new | new | | | | | |
| 33. | | chr16 | #Partially | #Partially | highly | YES | | | |
| | | 65229804 | deleted in | deleted in | active | | | | |
| | | 65235349 | new | new | | | | | |
| 34. | | chr20 | #Partially | #Partially | highly | YES | YES | | |
| | | 12340266 | deleted in | deleted in | active | | | | |
| | | 12345939 | new | new | | | | | |
| 35. | | chr20 | #Partially | #Partially | highly | YES | | | |
| | | 40269053 | deleted in | deleted in | active | | | | |
| | | 40274761 | new | new | | | | | |
| 36. | PCDH11X | chrX 92100239 | #Partially | #Partially | highly | YES | | | |
| | | 92105917 | deleted in | deleted in | active | | | | |
| | | | new | new | | | | | |
| 37. | | chrX | #Partially | #Split in | highly | YES | YES | | |
| | | 114466671 | deleted in | new | active | | | | |
| | | 114472531 | new | | | | | | |
| 38. | PCDH11Y | chrY 5324786 | #Partially | #Split in | highly | YES | YES | Nine | |
| | | 5330427 | deleted in | new | active | | | sites | |
| | | | new | | | | | | |
| 39. | | chr4 87921802 | #Split in | #Split in | highly | YES | | | |
| | | 87927246 | new | new | active | | | | |
| 40. | LOC102467213 | chr5 | #Split in | #Split in | highly | Bonobo | | | |
| | | 106978587 | new | new | active | | | | |
| | | 106984086 | | | | | | | |
| 41. | | chr1 | #Partially | #Partially | moderately | YES | | | |
| | | 183613209 | deleted in | deleted in | active | | | | |
| | | 183619373 | new | new | | | | | |
| 42. | | chr1 | #Partially | #Partially | moderately | YES | | | |
| | | 195847913 | deleted in | deleted in | active | | | | |
| | | 195848597 | new | new | | | | | |
| 43. | | chr1 | #Partially | #Split in | moderately | YES | | | |
| | | 218593627 | deleted in | new | active | | | | |
| | | 218600065 | new | | | | | | |
| 44. | | chr1 | #Partially | #Partially | moderately | YES | | | |
| | | 233683448 | deleted in | deleted in | active | | | | |
| | | 233689204 | new | new | | | | | |
| 45. | | chr1 5044795 | #Partially | #Partially | moderately | YES | YES | | |
| | | 5053098 | deleted in | deleted in | active | | | | |
| | | | new | new | | | | | |
| 46. | | chr1 55022707 | #Partially | #Partially | moderately | YES | | | |
| | | 55028369 | deleted in | deleted in | active | | | | |
| | | | new | new | | | | | |
| 47. | | chr1 64349942 | #Partially | #Partially | moderately | YES | | | |
| | | 64355761 | deleted in | deleted in | active | | | | |
| | | | new | new | | | | | |
| 48. | | chr1 68386003 | #Partially | #Partially | moderately | YES | | | |
| | | 68391992 | deleted in | deleted in | active | | | | |
| | | | new | new | | | | | |
| 49. | | chr1 72980445 | #Partially | #Partially | moderately | YES | | | |
| | | 72993602 | deleted in | deleted in | active | | | | |
| | | | new | new | | | | | |
| 50. | | chr1 99509510 | #Partially | #Partially | moderately | YES | YES | chr1: 99508046-99516831 8,786 bp | |
| | | 99515367 | deleted in | deleted in | active | | | | |
| | | | new | new | | | | | |
| 51. | | chr10 | #Partially | #Partially | moderately | YES | YES | | |
| | | 25768955 | deleted in | deleted in | active | | | | |
| | | 25774917 | new | new | | | | | |
| 52. | | chr10 | #Partially | #Partially | moderately | Gorilla | | | |
| | | 53492722 | deleted in | deleted in | active | | | | |
| | | 53493946 | new | new | | | | | |
| 53. | | chr10 | #Partially | #Partially | moderately | YES | Probable (gorilla) | | |
| | | 53500028 | deleted in | deleted in | active | | | | |
| | | 53504727 | new | new | | | | | |
| 54. | | chr10 | #Partially | #Partially | moderately | YES | | | |
| | | 54166675 | deleted in | deleted in | active | | | | |
| | | 54172501 | new | new | | | | | |
| 55. | | chr10 | #Partially | #Partially | moderately | YES | | | |
| | | 58860994 | deleted in | deleted in | active | | | | |
| | | 58867331 | new | new | | | | | |
| 56. | | chr10 | #Partially | #Partially | moderately | YES | | | |
| | | 90294982 | deleted in | deleted in | active | | | | |
| | | 90300722 | new | new | | | | | |
| 57. | | chr11 3470256 | #Split in | #Split in | moderately | YES | YES | 12 | |
| | | 3485187 | new | new | active | | | | |
| 58. | | chr11 6069821 | #Partially | #Partially | moderately | YES | | | |
| | | 6075884 | deleted in | deleted in | active | | | | |
| | | | new | new | | | | | |

| | | | | | | | |
|---|---|---|---|---|---|---|---|
| 59. | chr11 | #Split in | #Split in | moderately | YES | YES | chr11: 71733794-71756475 22,682 bp |
| | 71737574 | new | new | active | | | |
| | 71752695 | | | | | | |
| 60. | chr11 | #Partially | #Partially | moderately | YES | | |
| | 96587634 | deleted in | deleted in | active | | | |
| | 96593674 | new | new | | | | |
| 61. | chr12 | #Partially | #Partially | moderately | YES | | |
| | 17021893 | deleted in | deleted in | active | | | |
| | 17027363 | new | new | | | | |
| 62. | chr12 | #Partially | #Partially | moderately | YES | | |
| | 20762908 | deleted in | deleted in | active | | | |
| | 20769052 | new | new | | | | |
| 63. | chr12 | #Partially | #Partially | moderately | YES | | |
| | 20817907 | deleted in | deleted in | active | | | |
| | 20822617 | new | new | | | | |
| 64. | chr12 | #Split in | #Deleted | moderately | YES | | |
| | 67766803 | new | in new | active | | | |
| | 67772346 | | | | | | |

| | | | | | | |
|---|---|---|---|---|---|---|
| 65. | chr12 8279022 | #Split in | #Split in | moderately | YES | Probable (chimp) |
| | 8294090 | new | new | active | | |

| | | | | | | |
|---|---|---|---|---|---|---|
| 66. | chr12 | #Partially | #Deleted | moderately | YES | Probable (bonobo) |
| | 99715181 | deleted in | in new | active | | |
| | 99721737 | new | | | | |
| 67. | chr13 | #Partially | #Partially | moderately | YES | |
| | 109265089 | deleted in | deleted in | active | | |
| | 109271116 | new | new | | | |
| 68. | chr13 | #Partially | #Partially | moderately | YES | |
| | 34799253 | deleted in | deleted in | active | | |
| | 34803348 | new | new | | | |
| 69. | chr13 | #Partially | #Partially | moderately | YES | |
| | 48056343 | deleted in | deleted in | active | | |
| | 48062289 | new | new | | | |
| 70. | chr13 | #Partially | #Partially | moderately | YES | |
| | 86358167 | deleted in | deleted in | active | | |
| | 86364136 | new | new | | | |

| | | | | | | | |
|---|---|---|---|---|---|---|---|
| 71. | chr14 | #Partially | #Partially | moderately | YES | YES | chr14: 41514368-41523384 9,017 bp. |
| | 41515870 | deleted in | deleted in | active | | | |
| | 41521881 | new | new | | | | |
| 72. | chr15 | #Partially | #Partially | moderately | YES | | |
| | 52738557 | deleted in | deleted in | active | | | |
| | 52745204 | new | new | | | | |
| 73. | chr15 | #Partially | #Partially | moderately | YES | | |
| | 88547267 | deleted in | deleted in | active | | | |
| | 88551308 | new | new | | | | |

| 74. | chr16 | #Partially | #Partially | moderately | YES | Overlapping pattern when combine views of Chimp & Bonobo genomes |
|---|---|---|---|---|---|---|
| | 60078534 | deleted in | deleted in | active | | |
| | 60084578 | new | new | | | |
| 75. | chr16 | #Partially | #Partially | moderately | YES | |
| | 62979239 | deleted in | deleted in | active | | |
| | 62985208 | new | new | | | |
| 76. | chr16 8833042 | #Partially | #Partially | moderately | YES | |
| | 8845457 | deleted in | deleted in | active | | |
| | | new | new | | | |
| 77. | chr17 | #Partially | #Partially | moderately | YES | |
| | 11971755 | deleted in | deleted in | active | | |
| | 11976947 | new | new | | | |
| 78. | chr17 | #Split in | #Partially | moderately | YES | Probable (chimp) |
| | 34183190 | new | deleted in | active | | |
| | 34188994 | | new | | | |
| 79. | chr19 | #Partially | #Partially | moderately | YES | YES |
| | 22568269 | deleted in | deleted in | active | | |
| | 22575020 | new | new | | | |
| 80. | chr19 5548575 | #Partially | #Partially | moderately | YES | Overlapping pattern when combine views of Chimp & Bonobo genomes |
| | 5553212 | deleted in | deleted in | active | | |
| | | new | new | | | |
| 81. | chr2 12569679 | #Partially | #Partially | moderately | YES | |
| | 12575439 | deleted in | deleted in | active | | |
| | | new | new | | | |
| 82. | chr2 | #Split in | #Partially | moderately | YES | Probable (chimp) |
| | 165707551 | new | deleted in | active | | |
| | 165716198 | | new | | | |

| | | | | | | |
|---|---|---|---|---|---|---|
| 83. | chr2 | #Partially | #Partially | moderately | YES | Probable (bonobo) |
| | 187670482 | deleted in | deleted in | active | | |
| | 187676269 | new | new | | | |
| 84. | chr2 | #Partially | #Partially | moderately | YES | |
| | 192130385 | deleted in | deleted in | active | | |
| | 192136111 | new | new | | | |
| 85. | chr2 | #Partially | #Partially | moderately | YES | Probable (bonobo) |
| | 237606783 | deleted in | deleted in | active | | |
| | 237612654 | new | new | | | |

| | | | | | | | |
|---|---|---|---|---|---|---|---|
| 86. | chr2 57192262 | #Deleted | #Partially | moderately | YES | YES | chr2: 57190655-57200305 9,651 bp |
| | 57198696 | in new | deleted in | active | | | |
| | | | new | | | | |
| 87. | chr2 58314168 | #Partially | #Partially | moderately | YES | | |
| | 58319388 | deleted in | deleted in | active | | | |
| | | new | new | | | | |
| 88. | chr2 60417434 | #Partially | #Deleted | moderately | YES | | |
| | 60422485 | deleted in | in new | active | | | |
| | | new | | | | | |
| 89. | chr2 71086359 | #Partially | #Partially | moderately | YES | | |
| | 71090997 | deleted in | deleted in | active | | | |
| | | new | new | | | | |

| | | | | | | |
|---|---|---|---|---|---|---|
| 90. | chr2 77965139 | #Partially | #Partially | moderately | YES | Probable (bonobo) |
| | 77970850 | deleted in | deleted in | active | | |
| | | new | new | | | |
| 91. | chr20 | #Partially | #Partially | moderately | YES | |
| | 19752048 | deleted in | deleted in | active | | |
| | 19756776 | new | new | | | |
| 92. | chr20 | #Partially | #Partially | moderately | YES | |
| | 40093109 | deleted in | deleted in | active | | |
| | 40099009 | new | new | | | |

| | | | | | | | |
|---|---|---|---|---|---|---|---|
| 93. | chr22 | #Partially | #Partially | moderately | YES | YES | chr22: 16608907-16617551 8,645 bp |
| | 16611307 | deleted in | deleted in | active | | | |
| | 16615149 | new | new | | | | |
| 94. | chr3 | #Split in | #Split in | moderately | YES | | |
| | 125863749 | new | new | active | | | |
| | 125869497 | | | | | | |
| 95. | chr3 | #Partially | #Partially | moderately | YES | | |
| | 153226149 | deleted in | deleted in | active | | | |
| | 153232523 | new | new | | | | |
| 96. | chr3 16744185 | #Partially | #Partially | moderately | YES | | |
| | 16750064 | deleted in | deleted in | active | | | |
| | | new | new | | | | |
| 97. | chr3 | #Split in | #Partially | moderately | YES | | |
| | 170817614 | new | deleted in | active | | | |
| | 170823761 | | new | | | | |
| 98. | chr3 39577831 | #Partially | #Partially | moderately | YES | | |
| | 39583618 | deleted in | deleted in | active | | | |
| | | new | new | | | | |
| 99. | chr3 46246274 | #Partially | #Partially | moderately | YES | | |
| | 46252065 | deleted in | deleted in | active | | | |
| | | new | new | | | | |
| 100. | chr3 78581211 | #Partially | #Partially | moderately | YES | | |
| | 78588919 | deleted in | deleted in | active | | | |
| | | new | new | | | | |
| 101. | chr4 | #Partially | #Partially | moderately | YES | | |
| | 152741354 | deleted in | deleted in | active | | | |
| | 152747147 | new | new | | | | |
| 102. | chr4 16997746 | #Split in | #Partially | moderately | YES | | |
| | 17003925 | new | deleted in | active | | | |
| | | | new | | | | |
| 103. | chr4 | #Partially | #Partially | moderately | YES | | |
| | 172955659 | deleted in | deleted in | active | | | |
| | 172962312 | new | new | | | | |
| 104. | chr4 | #Partially | #Partially | moderately | YES | | |
| | 189479538 | deleted in | deleted in | active | | | |
| | 189485403 | new | new | | | | |

| | | | | | | |
|---|---|---|---|---|---|---|
| 105. | chr4 23722872 | #Partially | #Partially | moderately | YES | Probable (bonobo) |
| | 23727866 | deleted in | deleted in | active | | |
| | | new | new | | | |

| 106. | chr4 24500974 | #Partially | #Partially | moderately | YES | | | |
|---|---|---|---|---|---|---|---|---|
| | 24506750 | deleted in | deleted in | active | | | | |
| | | new | new | | | | | |
| 107. | chr4 3927445 | #Split in | #Split in | moderately | YES | YES | | |
| | 3933080 | new | new | active | | | | |
| 108. | chr5 | #Partially | #Partially | moderately | YES | | | |
| | 108548737 | deleted in | deleted in | active | | | | |
| | 108555018 | new | new | | | | | |
| 109. | chr5 | #Partially | #Partially | moderately | YES | | | |
| | 117046414 | deleted in | deleted in | active | | | | |
| | 117052246 | new | new | | | | | |
| 110. | chr5 | #Partially | #Split in | moderately | YES | | | |
| | 118947011 | deleted in | new | active | | | | |
| | 118952646 | new | | | | | | |
| 111. | chr5 12490211 | #Deleted | #Deleted | moderately | YES | YES | YES | chr5: 12489144-12495547 6,404 bp |
| | 12494480 | in new | in new | active | | | | |
| 112. | chr5 | #Partially | #Partially | moderately | YES | | | |
| | 170762080 | deleted in | deleted in | active | | | | |
| | 170767864 | new | new | | | | | |
| 113. | chr5 18535210 | #Partially | #Partially | moderately | YES | | | |
| | 18544018 | deleted in | deleted in | active | | | | |
| | | new | new | | | | | |
| 114. | chr5 84698674 | #Partially | #Partially | moderately | YES | | | |
| | 84704182 | deleted in | deleted in | active | | | | |
| | | new | new | | | | | |

| | | | | | | |
|---|---|---|---|---|---|---|
| 115. | chr5 92823741 | #Partially | #Deleted | moderately | YES | Probable (bonobo) |
| | 92829706 | deleted in | in new | active | | |
| | | new | | | | |
| 116. | chr6 | #Partially | #Deleted | moderately | YES | Probable (bonobo) |
| | 115031792 | deleted in | in new | active | | |
| | 115037619 | new | | | | |
| 117. | chr6 | #Partially | #Partially | moderately | YES | |
| | 120462506 | deleted in | deleted in | active | | |
| | 120468133 | new | new | | | |
| 118. | chr6 | #Partially | #Partially | moderately | YES | |
| | 121620421 | deleted in | deleted in | active | | |
| | 121626300 | new | new | | | |
| 119. | chr6 | #Partially | #Partially | moderately | YES | |
| | 122840216 | deleted in | deleted in | active | | |
| | 122845567 | new | new | | | |
| 120. | chr6 | #Partially | #Partially | moderately | YES | |
| | 124890406 | deleted in | deleted in | active | | |
| | 124897763 | new | new | | | |
| 121. | chr6 | #Partially | #Partially | moderately | YES | |
| | 131295356 | deleted in | deleted in | active | | |
| | 131301196 | new | new | | | |
| 122. | chr6 16259011 | #Partially | #Partially | moderately | YES | |
| | 16264893 | deleted in | deleted in | active | | |
| | | new | new | | | |
| 123. | chr6 18754143 | #Partially | #Partially | moderately | YES | |
| | 18759870 | deleted in | deleted in | active | | |
| | | new | new | | | |
| 124. | chr6 80482837 | #Partially | #Partially | moderately | YES | |
| | 80487823 | deleted in | deleted in | active | | |
| | | new | new | | | |
| 125. | chr7 | #Partially | #Partially | moderately | YES | |
| | 121563648 | deleted in | deleted in | active | | |
| | 121569668 | new | new | | | |
| 126. | chr7 | #Partially | #Partially | moderately | YES | |
| | 122816728 | deleted in | deleted in | active | | |
| | 122822998 | new | new | | | |
| 127. | chr7 51869849 | #Partially | #Partially | moderately | YES | |
| | 51872089 | deleted in | deleted in | active | | |
| | | new | new | | | |

| | | | | | | | | |
|---|---|---|---|---|---|---|---|---|
| 128. | chr8 | #Deleted | #Partially | moderately | YES | YES | YES | chr8: 104,284,367-104,293,639 9,273 bp |
| | 104285911 | in new | deleted in | active | | | | |
| | 104292093 | | new | | | | | |

| | | | | | | |
|---|---|---|---|---|---|---|
| 129. | chr8 | #Partially | #Partially | moderately | YES | Probable (bonobo) |
| | 114241603 | deleted in | deleted in | active | | |
| | 114247083 | new | new | | | |

| | | | | | | | |
|---|---|---|---|---|---|---|---|
| 130. | chr8 | #Partially | #Partially | moderately | YES | YES | chr8: 144,952,399-144,961,518 9,120 bp. |
| | 144953918 | deleted in | deleted in | active | | | |
| | 144959998 | new | new | | | | |
| 131. | chr8 79386105 | #Partially | #Partially | moderately | YES | | |
| | 79391685 | deleted in | deleted in | active | | | |
| | | new | new | | | | |
| 132. | chr8 81914410 | #Partially | #Partially | moderately | YES | | |
| | 81919889 | deleted in | deleted in | active | | | |
| | | new | new | | | | |

| | | | | | | |
|---|---|---|---|---|---|---|
| 133. | chr8 99943694 | #Partially | #Partially | moderately | YES | Probable (bonobo; chimp; gorilla) |
| | 99949609 | deleted in | deleted in | active | | |
| | | new | new | | | |
| 134. | chr9 | #Partially | #Partially | moderately | YES | |
| | 121790001 | deleted in | deleted in | active | | |
| | 121796769 | new | new | | | |
| 135. | chr9 99669780 | #Partially | #Partially | moderately | YES | |
| | 99675901 | deleted in | deleted in | active | | |
| | | new | new | | | |
| 136. | chrX | #Partially | #Partially | moderately | YES | |
| | 109866073 | deleted in | deleted in | active | | |
| | 109870862 | new | new | | | |

| 137. | chrX | #Partially | #Partially | moderately | YES | YES | chrX: 119,316,348-119,324,896 8,549 bp | | |
|---|---|---|---|---|---|---|---|---|---|
| | 119317772 | deleted in | deleted in | active | | | | | |
| | 119323471 | new | new | | | | | | |
| 138. | chrX 3553141 | #Partially | #Partially | moderately | YES | | | | |
| | 3560161 | deleted in | deleted in | active | | | | | |
| | | new | new | | | | | | |
| 139. | chrX 4540473 | #Partially | #Partially | moderately | YES | | | | |
| | 4546320 | deleted in | deleted in | active | | | | | |
| | | new | new | | | | | | |
| 140. | chrX 4891613 | #Partially | #Partially | moderately | YES | | | | |
| | 4897331 | deleted in | deleted in | active | | | | | |
| | | new | new | | | | | | |
| 141. | chr1 | #Deleted | #Partially | Inactive | YES | Probable (gorilla) | | | |
| | 104380122 | in new | deleted in | | | | | | |
| | 104388639 | | new | | | | | | |
| 142. | chr1 | #Deleted | #Deleted | Inactive | YES | Gorilla closest alignment | | | |
| | 108473289 | in new | in new | | | | | | |
| | 108478597 | | | | | | | | |
| 143. | chr1 | #Partially | #Split in | Inactive | YES | Gorilla closest alignment | | | 3 different loci in hg19 |
| | 120955898 | deleted in | new | | | | | | |
| | 120958127 | new | | | | | | | |
| 144. | chr1 | #Partially | #Split in | Inactive | YES | Gorilla closest alignment | | | 3 different loci in hg19 |
| | 120955898 | deleted in | new | | | | | | |
| | 120958127 | new | | | | | | | |
| 145. | chr1 | #Partially | #Split in | Inactive | YES | Gorilla closest alignment | | | 3 different loci in hg19 |
| | 120955898 | deleted in | new | | | | | | |
| | 120958127 | new | | | | | | | |
| 146. | chr1 | #Split in | #Partially | Inactive | YES | Gorilla closest alignment | | | |
| | 210187603 | new | deleted in | | | | | | |
| | 210195678 | | new | | | | | | |
| 147. | chr1 | #Partially | #Partially | Inactive | YES | | | | |
| | 228676558 | deleted in | deleted in | | | | | | |
| | 228682691 | new | new | | | | | | |
| 148. | chr1 22997504 | #Deleted | #Partially | Inactive | YES | | | | |
| | 23004403 | in new | deleted in | | | | | | |
| | | | new | | | | | | |
| 149. | chr1 37907814 | #Partially | #Split in | Inactive | YES | | | | |
| | 37914173 | deleted in | new | | | | | | |
| | | new | | | | | | | |
| 150. | chr1 70588436 | #Partially | #Partially | Inactive | YES | | | | |
| | 70593991 | deleted in | deleted in | | | | | | |
| | | new | new | | | | | | |
| 151. | chr1 84058413 | #Deleted | #Deleted | Inactive | YES | YES | YES | truncated LTR7/HERVH next to L1HS | |
| | 84058945 | in new | in new | | | | | | |
| 152. | chr10 | #Partially | #Partially | Inactive | YES | | | | |
| | 118893301 | deleted in | deleted in | | | | | | |
| | 118900351 | new | new | | | | | | |
| 153. | chr10 | #Partially | #Deleted | Inactive | YES | YES | YES | truncated LTR7/HERVH next to SVA_F | |
| | 17630036 | deleted in | in new | | | | | | |
| | 17632161 | new | | | | | | | |
| 154. | chr10 | #Partially | #Partially | Inactive | YES | Probable (chimp) | | | |
| | 25716420 | deleted in | deleted in | | | | | | |
| | 25722926 | new | new | | | | | | |
| 155. | chr10 | #Partially | #Partially | Inactive | YES | | | | |
| | 35401604 | deleted in | deleted in | | | | | | |
| | 35408752 | new | new | | | | | | |
| 156. | chr10 | #Partially | #Deleted | Inactive | YES | L1HS sequence | YES | L1HS human-specific insert within | |
| | 79963907 | deleted in | in new | | | insert | | LTR7/HERVH | |
| | 79968032 | new | | | | | | | |
| 157. | chr10 | #Partially | #Partially | Inactive | Crab-eating macaque | | | | |
| | 99260263 | deleted in | deleted in | | | | | | |
| | 99265383 | new | new | | | | | | |
| 158. | chr11 | #Partially | #Partially | Inactive | YES | L1PA2 sequence | YES | L1PA2 human-specific insert within | |
| | 122824427 | deleted in | deleted in | | | insert | | LTR7/HERVH | |
| | 122832822 | new | new | | | | | | |
| 159. | chr11 | #Split in | #Split in | Inactive | YES | | | | |
| | 123865321 | new | new | | | | | | |
| | 123871065 | | | | | | | | |
| 160. | chr11 | #Partially | #Partially | Inactive | Gorilla; Golden snub-nosed monkey | | | | |
| | 25326795 | deleted in | deleted in | | | | | | |
| | 25333699 | new | new | | | | | | |
| 161. | chr11 | #Partially | #Partially | Inactive | YES | | | | |
| | 29973621 | deleted in | deleted in | | | | | | |
| | 29977330 | new | new | | | | | | |
| 162. | chr11 4219298 | #Partially | #Partially | Inactive | YES | | | | |
| | 4225317 | deleted in | deleted in | | | | | | |
| | | new | new | | | | | | |
| 163. | chr11 4315701 | #Split in | #Split in | Inactive | YES | YES | YES | | |
| | 4321901 | new | new | | | | | | |
| 164. | chr11 | #Split in | #Split in | Inactive | YES | Orangutan closest | | | |
| | 67759684 | new | new | | | | | | |
| | 67765364 | | | | | | | | |
| 165. | chr11 | #Split in | #Split in | Inactive | YES | LTR2C/HERVE | YES | LTR2C/HERVE human-specific insert within | |
| | 67841905 | new | new | | | sequence insert | | LTR7/HERVH | |
| | 67856961 | | | | | | | | |
| 166. | chr12 | #Partially | #Partially | Inactive | YES | | | | |
| | 127153654 | deleted in | deleted in | | | | | | |
| | 127158069 | new | new | | | | | | |
| 167. | chr12 | #Partially | #Partially | Inactive | YES | | | | |
| | 132889510 | deleted in | deleted in | | | | | | |
| | 132898499 | new | new | | | | | | |
| 168. | chr12 | #Partially | #Partially | Inactive | YES | YES | | | |
| | 25163212 | deleted in | deleted in | | | | | | |
| | 25169515 | new | new | | | | | | |
| 169. | chr12 9962436 | #Partially | #Partially | Inactive | YES | | | | |
| | 9968690 | deleted in | deleted in | | | | | | |
| | | new | new | | | | | | |
| 170. | chr14 | #Partially | #Partially | Inactive | YES | | | | |
| | 31246361 | deleted in | deleted in | | | | | | |
| | 31251138 | new | new | | | | | | |
| 171. | chr14 | #Partially | #Split in | Inactive | YES | | | | |
| | 71124206 | deleted in | new | | | | | | |
| | 71130006 | new | | | | | | | |
| 172. | chr14_GL000009v2_random | #Partially | #Partially | Inactive | YES | chr14_GL009v2_random: | YES | truncated HERVH next to human-specific | |
| | 197844 199392 | deleted in | deleted in | | | 199,076-201,397 | | SVA_D insert | |
| | | new | new | | | 2,322 bp. | | | |
| 173. | chr15 | #Deleted | #Partially | Inactive | Geen monkey | | | | |
| | 41131295 | in new | deleted in | | | | | | |
| | 41137621 | | new | | | | | | |
| 174. | chr15 | #Partially | #Partially | Inactive | YES | | | | |
| | 90133292 | deleted in | deleted in | | | | | | |
| | 90138300 | new | new | | | | | | |
| 175. | chr16 | #Deleted | #Split in | Inactive | YES | | | | |
| | 70211765 | in new | new | | | | | | |
| | 70212791 | | | | | | | | |
| 176. | chr18 | #Partially | #Partially | Inactive | Gorilla | | | | |
| | 31284198 | deleted in | deleted in | | | | | | |
| | 31289927 | new | new | | | | | | |
| 177. | chr19 | #Deleted | #Deleted | Inactive | Orangitan/Gorilla | | | | |
| | 20376301 | in new | in new | | | | | | |
| | 20376564 | | | | | | | | |
| 178. | chr19 | #Deleted | #Partially | Inactive | YES | YES | YES | | |
| | 38750365 | in new | deleted in | | | | | | |
| | 38755295 | | new | | | | | | |
| 179. | chr19 | #Deleted | #Deleted | Inactive | Multiple species | | | | |
| | 46201640 | in new | in new | | | | | | |
| | 46203386 | | | | | | | | |
| 180. | chr19 | #Partially | #Partially | Inactive | YES | | | | |
| | 55122804 | deleted in | deleted in | | | | | | |
| | 55129538 | new | new | | | | | | |
| 181. | chr2 | #Partially | #Partially | Inactive | Gorilla; gibbon | | | | |
| | 110217883 | deleted in | deleted in | | | | | | |
| | 110220841 | new | new | | | | | | |
| 182. | chr2 | #Partially | #Partially | Inactive | Gorilla | | | | |
| | 117130628 | deleted in | deleted in | | | | | | |
| | 117135078 | new | new | | | | | | |
| 183. | chr2 | #Partially | #Partially | Inactive | YES | | | | |
| | 150112716 | deleted in | deleted in | | | | | | |
| | 150118564 | new | new | | | | | | |
| 184. | chr2 | #Partially | #Partially | Inactive | YES | Probable (orangutan) | | | |
| | 218174019 | deleted in | deleted in | | | | | | |
| | 218179886 | new | new | | | | | | |
| 185. | chr2 | #Partially | #Partially | Inactive | YES | | | | |
| | 224087353 | deleted in | deleted in | | | | | | |
| | 224093515 | new | new | | | | | | |
| 186. | chr2 | #Partially | #Partially | Inactive | YES | | | | |
| | 224296632 | deleted in | deleted in | | | | | | |
| | 224302363 | new | new | | | | | | |
| 187. | chr2 34789818 | #Partially | #Partially | Inactive | YES | | | | |
| | 34796056 | deleted in | deleted in | | | | | | |
| | | new | new | | | | | | |
| 188. | chr2 36599099 | #Partially | #Partially | Inactive | YES | | | | |
| | 36604761 | deleted in | deleted in | | | | | | |
| | | new | new | | | | | | |
| 189. | chr2 3815548 | #Partially | #Partially | Inactive | YES | YES | 28 sites | | |
| | 3821340 | deleted in | deleted in | | | | | | |
| | | new | new | | | | | | |
| 190. | chr2 71157777 | #Partially | #Partially | Inactive | YES | YES | YES | SVA_D human-specific insert within | |
| | 71165609 | deleted in | deleted in | | | | | LTR7/HERVH | |
| | | new | new | | | | | | |
| 191. | chr2 89048844 | #Split in | #Split in | Inactive | YES | | | | |
| | 89056967 | new | new | | | | | | |
| 192. | chr2 90143600 | #Split in | #Partially | Inactive | YES | | | | |
| | 90151719 | new | deleted in | | | | | | |
| | | | new | | | | | | |
| 193. | chr20 1727238 | #Deleted | #Deleted | Inactive | YES | | | | |
| | 1733570 | in new | in new | | | | | | |
| 194. | chr20 896876 | #Split in | #Split in | Inactive | YES | | | | |
| | 901599 | new | new | | | | | | |
| 195. | chr22 | #Partially | #Split in | Inactive | YES | | | | |
| | 39056261 | deleted in | new | | | | | | |
| | 39068308 | new | | | | | | | |
| 196. | chr3 1240736 | #Partially | #Partially | Inactive | YES | | | | |
| | 1245092 | deleted in | deleted in | | | | | | |
| | | new | new | | | | | | |
| 197. | chr3 | #Partially | #Split in | Inactive | YES | | | | |
| | 128829425 | deleted in | new | | | | | | |
| | 128842027 | new | | | | | | | |
| 198. | chr3 | #Partially | #Partially | Inactive | YES | | | | |
| | 133428173 | deleted in | deleted in | | | | | | |
| | 133434933 | new | new | | | | | | |
| 199. | chr3 | #Split in | #Partially | Inactive | YES | | | | |
| | 146353816 | new | deleted in | | | | | | |
| | 146367972 | | new | | | | | | |
| 200. | chr3 | #Partially | #Partially | Inactive | YES | | | | |
| | 162153420 | deleted in | deleted in | | | | | | |
| | 162159637 | new | new | | | | | | |
| 201. | chr3 | #Partially | #Partially | Inactive | Multiple species | | | | |
| | 168930919 | deleted in | deleted in | | | | | | |
| | 168933315 | new | new | | | | | | |
| 202. | chr3 | #Split in | #Split in | Inactive | YES | | | | |
| | 170672176 | new | new | | | | | | |
| | 170689306 | | | | | | | | |
| 203. | chr3 | #Partially | #Partially | Inactive | YES | | | | |
| | 178207402 | deleted in | deleted in | | | | | | |
| | 178214658 | new | new | | | | | | |
| 204. | chr3 | #Partially | #Partially | Inactive | YES | | | | |
| | 192071108 | deleted in | deleted in | | | | | | |
| | 192076858 | new | new | | | | | | |
| 205. | chr3 38070495 | #Partially | #Partially | Inactive | YES | | | | |
| | 38083728 | deleted in | deleted in | | | | | | |
| | | new | new | | | | | | |
| 206. | chr3 46387684 | #Split in | #Partially | Inactive | YES | | | | |
| | 46393402 | new | deleted in | | | | | | |
| | | | new | | | | | | |
| 207. | chr3 83354175 | #Partially | #Partially | Inactive | YES | | | | |
| | 83357600 | deleted in | deleted in | | | | | | |
| | | new | new | | | | | | |
| 208. | chr4 | #Partially | #Partially | Inactive | YES | YES | YES | 29 sites | Good example of the |
| | 115975699 | deleted in | deleted in | | | | | | insertion within low G/C |
| | 115981223 | new | new | | | | | | content region |
| 209. | chr4 | #Partially | #Partially | Inactive | Orangutan | | | | |
| | 167876311 | deleted in | deleted in | | | | | | |
| | 167882021 | new | new | | | | | | |
| 210. | chr4 | #Partially | #Partially | Inactive | YES | | | | |
| | 178207119 | deleted in | deleted in | | | | | | |
| | 178213342 | new | new | | | | | | |
| 211. | chr4 27974888 | #Partially | #Partially | Inactive | YES | YES | YES | LTR12C | Good example of the |
| | 27981374 | deleted in | deleted in | | | | | insert | insertion within low G/C |
| | | new | new | | | | | within | content region |
| | | | | | | | | LTR7/ | |
| | | | | | | | | HERVH | |
| 212. | chr4 68030945 | #Split in | #Partially | Inactive | YES | | | | |
| | 68037573 | new | deleted in | | | | | | |
| | | | new | | | | | | |
| 213. | chr4 71031809 | #Partially | #Deleted | Inactive | YES | YES | | | |
| | 71037274 | deleted in | in new | | | | | | |
| | | new | | | | | | | |

| | | | | | | | | |
|---|---|---|---|---|---|---|---|---|
| 214. | chr4 9094399 | #Split in | #Split in | Inactive | YES | YES | YES | HERVE/LTR2C insert within LTR7/HERVH |
| | 9108459 | new | new | | | | | |
| 215. | chr4 92025771 | #Partially | #Partially | Inactive | YES | | | |
| | 92031162 | deleted in | deleted in | | | | | |
| | | new | new | | | | | |
| 216. | chr5 | #Partially | #Partially | Inactive | YES | | | |
| | 108567660 | deleted in | deleted in | | | | | |
| | 108574883 | new | new | | | | | |
| 217. | chr5 | #Partially | #Partially | Inactive | YES | | | 2 copies of LTR7/HERVH placed in close |
| | 161240263 | deleted in | deleted in | | | | | proximity |
| | 161255013 | new | new | | | | | |
| 218. | chr5 702470 | #Partially | #Deleted | Inactive | YES | | | |
| | 708501 | deleted in | in new | | | | | |
| | | new | | | | | | |
| 219. | chr5 7055004 | #Partially | #Partially | Inactive | YES | | | |
| | 7063741 | deleted in | deleted in | | | | | |
| | | new | new | | | | | |
| 220. | chr5 76879900 | #Split in | #Partially | Inactive | YES | | | |
| | 76887017 | new | deleted in | | | | | |
| | | | new | | | | | |
| 221. | chr5 98080082 | #Partially | #Partially | Inactive | YES | | | |
| | 98088779 | deleted in | deleted in | | | | | |
| | | new | new | | | | | |
| 222. | chr6 | #Partially | #Partially | Inactive | YES | | | |
| | 164338768 | deleted in | deleted in | | | | | |
| | 164344779 | new | new | | | | | |
| 223. | chr6 | #Partially | #Deleted | Inactive | YES | | | |
| | 164652141 | deleted in | in new | | | | | |
| | 164658014 | new | | | | | | |
| 224. | chr6 29245476 | #Split in | #Partially | Inactive | Gorilla | | | |
| | 29252808 | new | deleted in | | | | | |
| | | | new | | | | | |

| | | | | | | |
|---|---|---|---|---|---|---|
| 225. | chr6 3167035 | #Partially | #Partially | Inactive | YES | Gorilla closest alignment (probable) |
| | 3173856 | deleted in | deleted in | | | |
| | | new | new | | | |
| 226. | chr6 51938240 | #Partially | #Partially | Inactive | YES | |
| | 51944426 | deleted in | deleted in | | | |
| | | new | new | | | |
| 227. | chr6 56010738 | #Partially | #Partially | Inactive | YES | |
| | 56016786 | deleted in | deleted in | | | |
| | | new | new | | | |

| | | | | | |
|---|---|---|---|---|---|
| 228. | chr6 65672767 | #Deleted | #Split in | Inactive | Orangutan; Gibbon; Green monkey |
| | 65673965 | in new | new | | |

| | | | | | | | | |
|---|---|---|---|---|---|---|---|---|
| 229. | chr6 67867627 | #Partially | #Split in | Inactive | YES | | | 2 copies of LTR7/HERVH placed in close |
| | 67889473 | deleted in | new | | | | | proximity |
| | | new | | | | | | |
| 230. | chr6 81343927 | #Partially | #Partially | Inactive | YES | YES | 33 sites | L1PA3 insert within LTR7Y/HERVH |
| | 81351160 | deleted in | deleted in | | | | | |
| | | new | new | | | | | |
| 231. | chr7 12659787 | #Split in | #Split in | Inactive | YES | | | |
| | 12665594 | new | new | | | | | |
| 232. | chr7 6948200 | #Split in | #Split in | Inactive | Chimp | | | HERVE insert within LTR7/HERVH |
| | 6962263 | new | new | | | | | |
| 233. | chr7 9457701 | #Deleted | #Partially | Inactive | YES | | | |
| | 9464218 | in new | deleted in | | | | | |
| | | | new | | | | | |

| | | | | | | |
|---|---|---|---|---|---|---|
| 234. | chr8 60305379 | #Partially | #Deleted | Inactive | YES | Gorilla closest alignment (probable) |
| | 60312009 | deleted in | in new | | | |
| | | new | | | | |
| 235. | chr8 7402289 | #Deleted | #Partially | Inactive | YES | |
| | 7408174 | in new | deleted in | | | |
| | | | new | | | |
| 236. | chr8 7903418 | #Deleted | #Partially | Inactive | YES | |
| | 7909304 | in new | deleted in | | | |
| | | | new | | | |
| 237. | chr9 | #Split in | #Deleted | Inactive | YES | |
| | 137843939 | new | in new | | | |
| | 137850465 | | | | | |
| 238. | chr9 35003292 | #Split in | #Partially | Inactive | YES | |
| | 35025134 | new | deleted in | | | |
| | | | new | | | |
| 239. | chr9 86146097 | #Partially | #Partially | Inactive | YES | |
| | 86148298 | deleted in | deleted in | | | |
| | | new | new | | | |

| | | | | | | | | |
|---|---|---|---|---|---|---|---|---|
| 240. | chr9 86586833 | #Deleted | #Deleted | Inactive | YES | YES | YES | Truncared LTR7Y/HERVH |
| | 86589057 | in new | in new | | | | | |

| | | | | | | |
|---|---|---|---|---|---|---|
| 241. | chr9 98265312 | #Split in | #Split in | Inactive | YES | Gibbon closest alignment |
| | 98271294 | new | new | | | |

| | | | | | |
|---|---|---|---|---|---|
| 242. | chrX | #Split in | #Deleted | Inactive | Gorilla; Orangutan |
| | 153094555 | new | in new | | |
| | 153101476 | | | | |

| | | | | | | |
|---|---|---|---|---|---|---|
| 243. | chrX 29975545 | #Partially | #Partially | Inactive | YES | Chimp closest alignment (probable) |
| | 29981247 | deleted in | deleted in | | | |
| | | new | new | | | |
| 244. | chrX 6272219 | #Partially | #Partially | Inactive | YES | |
| | 6277943 | deleted in | deleted in | | | |
| | | new | new | | | |

| | | | | | | | |
|---|---|---|---|---|---|---|---|
| 245. | chrX 64651095 | #Partially | #Deleted | Inactive | YES | YES | YES |
| | 64657665 | deleted in | in new | | | | |
| | | new | | | | | |

| | | | | | |
|---|---|---|---|---|---|
| 246. | chrX 75855965 | #Partially | #Partially | Inactive | Gorilla; Orangutan |
| | 75859573 | deleted in | deleted in | | |
| | | new | new | | |

| | | | | | |
|---|---|---|---|---|---|
| 247. | chrX 82726765 | #Partially | #Deleted | Inactive | Crab-eating macaque; baboon |
| | 82732949 | deleted in | in new | | |
| | | new | | | |

| | | | | | | |
|---|---|---|---|---|---|---|
| 248. | chrX 99158721 | #Partially | #Partially | Inactive | YES | Bonobo closest alignment (probable) |
| | 99165186 | deleted in | deleted in | | | |
| | | new | new | | | |

| 249. | chrY 10047167 | #Deleted | #Deleted | Inactive | YES | YES | YES | | |
|---|---|---|---|---|---|---|---|---|---|
| | 10053754 | in new | in new | | | | | | |
| 250. | chrY 14350504 | #Partially | #Split in | Inactive | Chimp | | | HERV9 next to HERVH/LTR7 | |
| | 14360015 | deleted in | new | | | | | | |
| | | new | | | | | | | |
| 251. | chrY 15769836 | #Split in | #Split in | Inactive | YES | YES (probable) | | truncated HERV9 next to HERVH/LTR7; LTR5_Hs | |
| | 15773029 | new | new | | | | | nearby | |
| 252. | chrY | #Deleted | #Deleted | Inactive | YES | YES | YES | Several | chrY: 20,998,615-21,208,449 |
| | 21035919 | in new | in new | | | | | adjacent | 209,835 bp |
| | 21045245 | | | | | | | copies of | |
| | | | | | | | | LTR7/ | |
| | | | | | | | | HERVH | |

| | | | | | | |
|---|---|---|---|---|---|---|
| 253. | chrY 7500589 | #Deleted | #Partially | Inactive | Chimp | smal Alu human-specific insert |
| | 7507138 | in new | deleted in | | | |
| | | | new | | | |
| 254. | | | | | | |

| | |
|---|---|
| 255. | 39 human-specific integration sites |
| 256. | 4 additional sites with other repeats involved |

(Section B, with rows continued)

| 1. | Human-specific deletions of ancestral DNA (size, bp) | | | | | |
|---|---|---|---|---|---|---|
| 2. | Chimp | Bonobo | Gorilla | Orangutan | Gibbon | Deleted chimp sequences |
| 3. | | | | | | |

| | | | | | |
|---|---|---|---|---|---|
| 4. | 12 | | | | ttgaaggtgagg (SEQ ID NO: 25); ctt; t; gtt |
| 5. | | | | | |
| 6. | 7,433 | 4 | 6,995; 4655 | 71,036 | |

| | | | | | | |
|---|---|---|---|---|---|---|
| 7. | | 2,647 | | | | |
| 8. | 1,187 | 3,179 | 5,054 | | | |
| 9. | | 20 | | | | |
| 10. | 4,462 | 5,110 | | | | |
| 11. | 1,314 | 2,323 | | | | |
| 12. | 7,599 | | | 13,298 | 143 | |
| 13. | 332 | | | | | |
| 14. | | 7,007 | 7,477 | 1,255 | | |
| 15. | 4 | 2 | | 5 | | |
| 16. | 6,003 | | 2,377 | | 892 | |
| 17. | 3,355 | 1,781 | | | | |
| 18. | 1,691 | | 2,552 | 11 | | |
| 19. | 192 | | 4,925 | | | |
| 20. | | 20 | | | | |
| 21. | 4 | 4 | 4 | 4; 5 | | |
| 22. | 87 | 2,437 | | | | |
| 23. | 5,679 | | 5,858 | | | |
| 24. | | 148 | 4,808 | | | |
| 25. | 600 | 3,376 | | | | |
| 26. | | 6,080 | | | | |
| 27. | 2,549 | 2,287 | 5,677 | | | |
| 28. | 21 | | 5,356 | | | |
| 29. | | 20 | 6,230 | | | |
| 30. | | 20 | 2,728 | | | |
| 31. | 9 | 2,931 | | | | |
| 32. | 3,862 | | | | | |
| 33. | | 20 | | | | |
| 34. | | 3,391 | | | | |
| 35. | | 1,542 | 4,257 | | | |
| 36. | | 1,331 | 8,338 | | | |
| 37. | 9,025 | 3,148 | 5,927 | | | |
| 38. | | 4,676 | 9,965 | | | |
| 39. | 31 | 8,555 | | 31 | | |
| 40. | 10 | | | | | |
| 41. | 444 | 20 | | | | |
| 42. | | | | | | |
| 43. | | | | | | |
| 44. | 2,635 | 51 | | | | |
| 45. | 13,562 | 14,752 | 17,588 | 8,519 | 9,799 | |
| 46. | | | 7,017 | | | |
| 47. | | | | | | |
| 48. | | | | | | |
| 49. | 2,726 | | 383 | | | |
| 50. | | 5,036 | | | | |
| 51. | | 2,775 | | | | |
| 52. | 10,267 | 9,951 | | | | |
| 53. | | 29 | 71 | | | |
| 54. | 4,696 | 21 | | | | |
| 55. | 2,249 | | 5,409 | | | |
| 56. | 873 | 2,846 | | | | |
| 57. | 5,907 | 5,854 | | | | |
| 58. | 4,635 | 4,270 | 4,286 | | | |
| 59. | 2,841 | 16,377; 11,640 | 13,109 | 2 | 523 | |
| 60. | | | | | | |
| 61. | | 281 | 4,665 | | 100 | |
| 62. | 4,691 | | 10,410 | 18,729 | | |
| 63. | | | | | | |
| 64. | | | 10 | | | |
| 65. | 7,353 | 14,276 | 75,171 | | | |
| 66. | | 2,024 | | | 5,004 | |
| 67. | | | | | | |
| 68. | | 2,016 | 2,304 | | | |
| 69. | 378 | | 4,821 | | | |
| 70. | | | 4,429 | | | |
| 71. | 3,175 | | 6,977 | | | |
| 72. | | | | 207 | | |
| 73. | 1,442 | 9,145 | 13,816 | 473 | | |
| 74. | 3,250 | 72 | 5,995 | 857 | | |
| 75. | 2,974 | 21 | 5,217 | | | |
| 76. | 14,642 | 775 | 14,698 | 12,302 | 4 | |
| 77. | 2,252 | | | | | |
| 78. | | 3,162 | | | | |
| 79. | 5,907 | | 12,891 | | | |
| 80. | 2,823 | 2,366 | | 6 | | |
| 81. | | 6,118 | | | | |
| 82. | | | | | | |
| 83. | | 4,030 | | | 38 | |
| 84. | | 20 | 5,682 | 10 | | |
| 85. | | 2,041 | | 15,075 | | |
| 86. | 5,376 | 5,184 | 100 | 2 | 9 | |
| 87. | | | | | | |
| 88. | | 980 | 3,238 | 115 | 95 | |
| 89. | 756 | | | | 511 | |
| 90. | 4,717 | 3,158 | 5,196 | | | |
| 91. | | 25 | | | | |
| 92. | | 20 | | | | |
| 93. | | 330 | 407 | | | |
| 94. | | 10,696 | | | | |
| 95. | 1,457 | 676 | 5,066 | 39 | 1,762 | |
| 96. | 10 | | 2,238 | 4,780 | | |
| 97. | | | | | | |
| 98. | | 3,159 | 8,055 | | | |
| 99. | 4,423 | | | | | |
| 100. | 5,871 | | 6,576 | | | |
| 101. | 5,980 | 2,517 | | | | |
| 102. | | | | 8,310 | | |
| 103. | 1,372 | 21 | 3,975 | | | |
| 104. | | | | | | |
| 105. | 5,431 | 3,625 | 4,346 | | | |
| 106. | | 20 | | | | |
| 107. | | 81,108 | | 35,326 | | |
| 108. | 10,133 | 12,135 | 115 | | 102 | |
| 109. | 3,436 | 19 | | | | |
| 110. | | | 12 | 10 | | |
| 111. | | | 2,637 | | 1,255 | |
| 112. | 444 | | | 3,918 | | |
| 113. | | 20 | | | | |
| 114. | | 22 | | | | |
| 115. | | 3,035 | | | | |
| 116. | | | | | | |
| 117. | 3,248 | 1,090 | 3,133 | | | |
| 118. | 2,526 | | 8,138 | | | |
| 119. | | | 2,486 | | | |
| 120. | 20 | 4,021 | 52 | | | |
| 121. | 21 | 2,983 | | | | |
| 122. | 2,469 | 240 | 4,807 | 2,025 | | |
| 123. | 2,849 | | 7,230 | | 17 | |
| 124. | 3,374 | | | | | |
| 125. | | | 120 | 31 | 101 | |
| 126. | 21 | | | | | |
| 127. | 10 | 2,480 | 3,759 | 4,838 | 5,037 | |
| 128. | 8,318 | | 3,998 | 595 | 5 | |
| 129. | | 3,148 | | 58 | 21 | |
| 130. | | | 9,101 | 1,875 | 3,228 | |
| 131. | | 21 | | | | |
| 132. | 3,017 | | 5,622 | | | |
| 133. | 2,250 | 3,244 | 3,619 | | | |
| 134. | | | | | | |
| 135. | 5,601 | 2,552 | 5,161 | | | |
| 136. | | | | | | |
| 137. | | | 4,180 | | | |
| 138. | 5211; | 3,051 | 5,773 | | | |
| | 1,148 | | | | | |
| 139. | 189 | 3,956 | 4,479 | | | |
| 140. | Chimp | Bonobo | Gorilla | Orangutan | Gibbon | Deleted chimp sequences |

| | |
|---|---|
| 141. | 10 |
| 142. | 525 |

| | | | | | | | |
|---|---|---|---|---|---|---|---|
| 143. | 6,043 | 46,624; 633 | chr1 | 1.44E+08 | 1.44E+08 | inactive | hg19 |
| 144. | 6,043 | 46,624; 633 | chr1 | 1.44E+08 | 1.44E+08 | inactive | hg19 |
| 145. | 6,043 | 46,624; 633 | chr1 | 1.5E+08 | 1.5E+08 | inactive | hg19 |

| | | | | | | |
|---|---|---|---|---|---|---|
| 146. | | | 4,520 | 2,512 | 2,088 | |
| 147. | 2,724 | | 3,324 | | | |
| 148. | 5,808 | | | 16,505 | | |
| 149. | | | | | | |
| 150. | 4,498 | | 5,484 | | | |
| 151. | | | 10,542 | 320 | | chr1: 84,050,744-84,059,836 9,093 bp Expanded |
| | | | | | | region |
| 152. | | | 10 | 1,189 | | |
| 153. | | | 219 | | | chr10: 17,627,912-17,632,693 4,782 bp. |
| | | | | | | Expanded region |
| 154. | 3,989 | | 5,001 | | 4,612 | |
| 155. | 5,336 | | | | 1,768 | |
| 156. | | | 1,567 | | 3,498 | |
| 157. | 5,039 | 6,326 | 5,174 | | | |
| 158. | | | 8,684 | | 288 | |
| 159. | 3,029 | 63 | | 6,729 | | |
| 160. | 1,693 | | | 1,115 | 1 | |
| 161. | | | | | | |
| 162. | 10 | | | | | |
| 163. | | | | 5,753 | | |
| 164. | 3,123 | 12.374 | | | | |
| 165. | 1,150 | | | | | |
| 166. | | 11,376 | 6,333 | | | |
| 167. | | | | 991 | 20; | |
| | | | | | 5,067 | |
| 168. | | 10,245 | | | | |
| 169. | | | | 1 | | |
| 170. | 17 | | | | | |
| 171. | | | 10 | 9,369 | 1,778 | |
| 172. | | | | | | chr14_GL000009v2_random: 198,560-200,881 2,322 |
| | | | | | | bp. Adjusted region |
| 173. | 710 | | | 5,783 | | |
| 174. | | | | | | |
| 175. | | 398; 651; 630 | 1,282 | | | chr16: 70,207,530-70,219,220 11,691 bp Adjusted |
| | | | | | | region |
| 176. | | | 1,521 | 4,063 | 1,334 | |

| | | | |
|---|---|---|---|
| 177. | 1537; 7,891 | 9,784; 2; 8,096; | chr19: 20,372,488-20,380,377 7,890 bp. Adjusted |
| | | | region |

| | | | | | | |
|---|---|---|---|---|---|---|
| 178. | 6,565 | 8,205 | 4; 1,809; 8,688 | 1,158; 8; | 4; | chr19: 38745436-38760225 14,790 bp. Expanded |
| | | | | 4; 24,024 | 5,641; | region |
| 179. | 21 | | 132; 176; 748; | 386; 9 | 18 | chr19: 46199895-46205132 5,238 bp. Expanded |
| | | | 3,064 | | | region |
| 180. | | 4,165 | 5,765 | 6,106 | 7,508; 1 | |
| 181. | | | | 584 | | |
| 182. | | | 12 | | | |
| 183. | 1; 1,297; | 1; 9 | | 1; 3,608; | 103 | |
| 184. | | | | | | |
| 185. | | | | 10 | 6,221 | |

| | | |
|---|---|---|
| 186. | 10; 1,684; 44 | 101 |

| 187. | 822 | 7 | | 4,274 | 9.248 |
|---|---|---|---|---|---|
| 188. | | 2,171 | | 18 | |
| 189. | | | | | |
| 190. | 19 | 3,298 | | | |
| 191. | 2,140 | | 123; 6,257 | 1,062 | 4; 6,903; 1 |
| 192. | 2,140 | 4,632; 20 | 63,864; 1,087; | 1,058 | 1; |
| | | | 717 | | 6,903; |

| | |
|---|---|
| 193. | 3,373; 1,549 |

| | | | |
|---|---|---|---|
| 194. | 5 | 1; 10; 638; | 1; 1; |
| | | | 100; |

| | | | | |
|---|---|---|---|---|
| 195. | 9,680 | 1 | 12,071 | 2; 16,223; |
| 196. | 11; 1,335 | 3,188 | | |

| | | | | | |
|---|---|---|---|---|---|
| 197. | | acc | | 4,863 | |
| 198. | 13 | | | 11 | 100 |
| 199. | ccgc | c | 3,814 | | 99,980 |

| | | | |
|---|---|---|---|
| 200. | gagagataatgggcgatgtttctcagggctgctt | 13,965 | 8,059; |
| | c (SEQ ID NO: 26) | | gagagataatgggcgatgtttctcagggctgcttc |

| | | | | | | |
|---|---|---|---|---|---|---|
| 201. | 3 | | | 315; 273 | 6,753 | chr3: 168928524-168935711 7,188 bp. |
| | | | | | | Expanded region |
| 202. | 4,589 | 7,997 | 5,833 | 6,335 | 100 | |
| 203. | | | 7,520 | | 4,027 | |
| 204. | 4,027; 25 | | | 6,842 | | |
| 205. | | | 12,257 | | | |
| 206. | 23; | 25; | | | | |
| 207. | 3,018 | 4,546 | 7,873 | 2,241 | 2,122 | |
| 208. | | | 5,797 | 53 | 686 | |
| 209. | 2,801 | | | 875 | | |
| 210. | | | 4,997 | 10 | | |
| 211. | | | | | | |
| 212. | 2,358 | | | 2 | | |
| 213. | | 9,053 | | 8,542 | | |
| 214. | | | | | | |
| 215. | | 1,490 | 172 | | | |
| 216. | 4,179 | 3,689 | 9,780 | | | |

| | | | |
|---|---|---|---|
| 217. | 3,408 | 10; 5,898; | 100; |

| | | | | | |
|---|---|---|---|---|---|
| 218. | | | 243 | | |
| 219. | 5,094 | | | 4,625 | |
| 220. | | | 245; 18 | 4,863 | |
| 221. | 771; | 2,274; | 6,002; | 1,600; | 1,454; 681; |
| | | | | 672; | |
| 222. | | | | 722 | |
| 223. | | 58 | | | |
| 224. | | | | 2,037; | |
| 225. | | 8,017; | 4,780; | 11,615; | 1,985; |
| 226. | | | | | 509 |
| 227. | 4,363 | | | 3,724 | |
| 228. | | | 5,224 | | |

| | | | | |
|---|---|---|---|---|
| 229. | | 6; | 2,837; | 1,317; 2,410; |
| 230. | 4,827 | | | |

| | | | | | |
|---|---|---|---|---|---|
| 231. | | | | 9; 10; | |
| 232. | 842; 64; | 838; | 63; 2,988; | 48 | |
| 233. | 8; | | | 8,118; | |
| 234. | | | 1,995 | | |
| 235. | 18; | | 2,406 | | |
| 236. | 18; | | | | |
| 237. | | | | | 100; |
| 238. | 17; | 4,546; 21; 6,817; | 6,024; 5,203; | 1; 838; | 105; 1; |
| | 5,228; | | 10,432; 203; | 1,352; | |
| 239. | 13,639; | 13,378; | | 19,228; | 1,056; |
| 240. | | | | | |
| 241. | 5,724; | 3,587; | 2,597; | 3,748 | |

| | | |
|---|---|---|
| 242. | 2,098; | 28; 168; 2,625; |

| | | | | | |
|---|---|---|---|---|---|
| 243. | 5,846; | 395; 1,007; | 12,569; | 6,571; | 869; |
| 244. | 4,236; | | | | |
| 245. | | | | | |
| 246. | | | | | |
| 247. | | 840; | 5,563; | 6,017; | |
| 248. | | 493; | | | |
| 249. | | | | | |
| 250. | | | | | |
| 251. | | | | | |
| 252. | | | | | |
| 253. | | | | | |
| 254. | | | | | |
| 255. | | | | | |
| 256. | | | | | |

TABLE 11
Human-specific SCARs defined based on the failures of the reciprocal alignments from the genomes of Chimpanzee and Bonobo to the human genome.
(Section A)

| 1. | Human-specific SCARs defined based on the failures of the reciprocal alignments from the genomes of Chimpanzee and Bonobo to the human genome | | | | | | | | | | |
|---|---|---|---|---|---|---|---|---|---|---|---|
| 2. | | 6 Reciprocal conversion failures (highly active) | | | | | | | | | |
| 3. | Gene | hg38 | | | LiftOver to Chimp | | | Reciprocal to hg19 | | | |
| 4. | | chr1 | 2.23E+08 | 2.23E+08 | chr1 | 2.02E+08 | 2.02E+08 | #Partially deleted in new | | | |
| 5. | | chr4 | 1.79E+08 | 1.79E+08 | #Partially deleted in new | | | | | | |
| 6. | MGC32805 | chr5 | 1.22E+08 | 1.22E+08 | #Partially deleted in new | | | | | | |
| 7. | | chr9 | 1.18E+08 | 1.18E+08 | #Partially deleted in new | | | | | | |
| 8. | | chr20 | 13357879 | 13362689 | #Partially deleted in new | | | | | | |
| 9. | | chrY | 5941110 | 5946036 | chrX | 92875117 | 92879988 | chrX | 92079426 | 92084344 | |
| 10. | | | | | | | | | | | |
| 11. | | 6 Bonobo failures of reciprocal to hg38 (from 75 converted) | | | | | | | | | |
| 12. | | 2 converted to Chimp but failed reciprocal conversion | | | | | | | | | |
| 13. | | | | | | | | | | | |
| 14. | | 25 Reciprocal conversion failures (moderately active) | | | | | | | 2 of 24 conserved in Chimp | | |
| 15. | | 24 failed reciprocal LiftOver Bonobo to hg38 | | | | PanTro LiftOver | | | Reciprocal from Chimp to hg19 | | |
| 16. | | #Partially deleted in new | chr1 | 5114346 | 5118888 | #Deleted in new | | | | | |
| 17. | | #Partially deleted in new | chr1 | 1.88E+08 | 1.88E+08 | #Partially deleted in new | | | | | |
| 18. | | #Partially deleted in new | chr1 | 2.29E+08 | 2.29E+08 | #Partially deleted in new | | | | | |
| 19. | | #Partially deleted in new | chr1 | 2.32E+08 | 2.32E+08 | #Partially deleted in new | | | | | |
| 20. | | #Partially deleted in new | chr3 | 78323653 | 78331379 | #Partially deleted in new | | | | | |
| 21. | | #Partially deleted in new | chr3 | 98191087 | 98196791 | #Partially deleted in new | | | | | |
| 22. | | #Partially deleted in new | chr3 | 1.25E+08 | 1.25E+08 | #Deleted in new | | | | | |
| 23. | | #Partially deleted in new | chr3 | 1.92E+08 | 1.92E+08 | #Partially deleted in new | | | | | |
| 24. | | #Partially deleted in new | chr4 | 97136991 | 97140080 | chr4 | 99566900 | 99569989 | Yes | | |
| 25. | | #Partially deleted in new | chr5 | 1.04E+08 | 1.04E+08 | #Split in new | | | | | |
| 26. | | #Partially deleted in new | chr5 | 1.69E+08 | 1.69E+08 | chr5 | 1.7E+08 | 1.7E+08 | #Partially deleted in new | | |
| 27. | | #Partially deleted in new | chr11 | 42120988 | 42125302 | #Partially deleted in new | | | | | |
| 28. | | #Partially deleted in new | chr12 | 1.03E+08 | 1.03E+08 | #Split in new | | | | | |
| 29. | | #Partially deleted in new | chr13 | 66141331 | 66147036 | #Partially deleted in new | | | | | |
| 30. | | #Partially deleted in new | chr15 | 36285827 | 36293371 | #Partially deleted in new | | | | | |
| 31. | | #Partially deleted in new | chr16 | 86278094 | 86281279 | #Partially deleted in new | | | | | |
| 32. | | #Partially deleted in new | chr17 | 75252620 | 75258281 | #Partially deleted in new | | | | | |
| 33. | | #Partially deleted in new | chr18 | 31803782 | 31810056 | #Partially deleted in new | | | | | |
| 34. | | #Partially deleted in new | chr18 | 73324614 | 73330362 | #Partially deleted in new | | | | | |
| 35. | | #Partially deleted in new | chrX | 16179201 | 16184434 | #Partially deleted in new | | | | | |
| 36. | | #Partially deleted in new | chrX | 92824427 | 92829345 | chrX | 92875117 | 92879988 | Yes | | |
| 37. | | #Partially deleted in new | chrX | 1.17E+08 | 1.17E+08 | #Partially deleted in new | | | | | |
| 38. | | #Split in new | chr4 | 9638974 | 9643702 | #Split in new | | | | | |
| 39. | | #Split in new | chr13 | 90839563 | 90854278 | #Partially deleted in new | | | | | |
| 40. | | | | | | | | | | | |
| 41. | | 3 of 15 failed reciprocal LiftOver from Chimp genome (from 15 Chimp LiftOver derived from 115 Bonobo primary LiftOver failures) | | | | | | | | | |
| 42. | | #Partially deleted in new | chr12 4018540 4023694 | | | chr12 | 4116781 | 4122861 | #Partially deleted in new | | |
| 43. | | #Partially deleted in new | chr22 32502753 32508503 | | | chr22 | 31171458 | 31177690 | #Partially deleted in new | | |
| 44. | | #Partially deleted in new | chr7 113234430 113239308 | | | chr7 | 1.15E+08 | 1.15E+08 | #Partially deleted in new | | |
| 45. | | | | | | | | | | | |
| 46. | | | | | | | | | | | |
| 47. | | 20 Reciprocal conversion failures (inactive) | | | | | | | | | |
| 48. | | 20 records of reciprocal converison | | | HUMAN_SPECIC | HUMAN_SPECIC | High | HUMAN_SPECIC | | | Chimp |
| | | failures (18 Bonobo; 2 Chimp) | | | INSERTIONS | INTEGRATION | Confidence | INTEGRATION SITE | | | |
| | | | | | | SITE | | | | | |
| 49. | | chr1 | 2.1E+08 | 2.1E+08 | Bonobo | | | | | | 4,748 |
| 50. | | chr2 | 57227241 | 57235205 | Bonobo | | | | | | |
| 51. | | chr2 | 1.42E+08 | 1.42E+08 | Bonobo | | | | | | 3,487 |
| 52. | | chr2 | 1.91E+08 | 1.91E+08 | Chimp; Bonobo; Gorilla; Gibbon | | | | | | |
| 53. | | chr3 | 97272462 | 97277550 | Bonobo | | | | | | 3,026 |
| 54. | | chr3 | 1.42E+08 | 1.42E+08 | Bonobo | | | | | | 6,558 |
| 55. | | chr6 | 33345061 | 33351803 | Gorilla | | | | | | |
| 56. | | chr10 | 1.01E+08 | 1.01E+08 | Bonobo | | | | | | |
| 57. | | chr15 | 94750748 | 94760376 | Bonobo | | | | | | |
| 58. | | chr19 | 46102204 | 46109320 | Bonobo | | | | | | 8,946; 15; |
| 59. | | chrX | 75631546 | 75637730 | Multiple species | | | | | | 626; |
| 60. | | chrX | 1.49E+08 | 1.49E+08 | Bonobo | | | | | | 2,768 |
| 61. | | chr1 | 1.7E+08 | 1.7E+08 | Bonobo | | | | | | 1,507 |
| 62. | | chr4 | 4001143 | 4005763 | Bonobo | | | | | | |
| 63. | | chr4 | 1.29E+08 | 1.29E+08 | Bonobo | | | | | | 4,256 |
| 64. | | chr6 | 40861971 | 40868133 | Bonobo | | | | | | |
| 65. | | chr12 | 1.14E+08 | 1.14E+08 | Bonobo | | | | | | |
| 66. | | chr19 | 5847388 | 5857653 | YES | | | | | | |
| 67. | | chr10 118893301 118900351 | | | YES | | | | | | |
| 68. | | chr10 25716420 25722926 | | | YES | | | | | | 3,989; |
| 69. | | | | | | | | | | | |

| | |
|---|---|
| 70. | 16 failed reciprocal Bonobo and direct Chimp conversion |

| | |
|---|---|
| 71. | 2 failed reciprocal Bonobo; converted to Chimp; failed reciprocal Chimp |

| | |
|---|---|
| 72. | 1 record failed reciprocal Bonobo; converted direct and reciprocal Chimp (Conserved in Chimp) |

| | |
|---|---|
| 73. | 2 records failed reciprocal conversion in Chimp (from 25 direct conversion to Chimp from 113 direct Bonobo failures) |

(Section B, with rows continued)

| 1. | Human-specific deletions of ancestral DNA (size, bp) | | | | |
|---|---|---|---|---|---|
| 2. | Chimp | Bonobo | Gorilla | Orangutan | Gibbon |
| 3. | 974 | 976 | 7,256 | | |
| 4. | | 60; 83; 61 | 1,926 | | |
| 5. | 2,113 | 211 | | | |
| 6. | 1,945 | 200 | 84; 235 | | |
| 7. | 3,233 | 1,008 | 10 | | |
| 8. | | 58; 409; 187; 2,587 | | | |
| 9. | | | | | |
| 10. | | | | | |
| 11. | | | | | |
| 12. | | | | | |
| 13. | Human-specific DNA loss (C & B) | | | | |
| 14. | Chimp | Bonobo | Gorilla | Orangutan | Gibbon |
| 15. | 6 | 4; 6 | 6 | 6 | 6 |
| 16. | 2,670 | 313 | | | |
| 17. | 35 | 890 | | | |
| 18. | | 560 | | | |
| 19. | 6,916 | 3,395 | | | |
| 20. | 2,491 | 663 | 5,501 | | |
| 21. | | 311 | | | |
| 22. | 4,083 | 318 | 6,124 | 5,125 | 3,680 |
| 23. | | 2,223 | | 74 | |
| 24. | 7,491 | 375 | 7,205 | | |
| 25. | | | | | 3,223 |
| 26. | | 1,785 | 9,265 | | |
| 27. | 100 | 7 | | 7 | 7; 14 |
| 28. | 1,442 | 1,229 | 4,733 | | |
| 29. | 2,247; 77 | | | | 829 |
| 30. | 3,089 | 251 | | | |
| 31. | 3; 18 | 61; 3; 3; 18 | | 737; 85; 3; 18 | |
| 32. | | 413 | 6,949 | 18 | |
| 33. | 3; 4; 5; 2 | 4; 65; 3; 2; 2; 3; 4; 5; 2 | 4,963 | | |
| 34. | 8,762 | 610 | 5,829 | 1,793 | 105 |
| 35. | | | 124; 96 | | |
| 36. | | 450 | | | |
| 37. | | 2; 3 | | | 2 |
| 38. | 16 | 780 | | | |
| 39. | | | | | |
| 40. | | | | | |
| 41. | 948 | | | | |
| 42. | 319 | 2,497 | | | |
| 43. | 535 | 3,647 | | | |
| 44. | | | | | |
| 45. | | | | | |
| 46. | | | | | |
| 47. | Bonobo | Gorilla | Orangutan | Gibbon | |
| 48. | 2; 1,316; | 2; 11; | 3; 10; | | |
| 49. | 2,887; | | | 6,327 | |
| 50. | 1,329 | | 25; 6 | | |
| 51. | | | 1,004 | | |
| 52. | 933 | | 567 | 3,483 | |
| 53. | 1,330 | 1,301, 11; | | 306 | |
| 54. | | 294 | | | |
| 55. | 2,667; | | 2,843; | 2,303; | |
| 56. | 1,247; | | | | |
| 57. | 5,555 | 1,472 | 97,124 | 662 | |
| 58. | 1,330; | | 3; 10; | 21,153; | |
| 59. | 966; | | 13,007; | | |
| 60. | 1,155; | 1,959; | | | |
| 61. | | 773 | 11 | | |
| 62. | 2,000; | 4,603; | 18; 847; | | |
| 63. | | | | | |
| 64. | 1,024; | 1; | | 1; 4,671; | |
| 65. | 3,398; | 5,264; | 5,882; 320; | 6,590; 1,889; | |
| 66. | | 10; | 1,189; | | |
| 67. | | 5,001; | | 4,612; | |
| 68. | | | | | |
| 69. | | | | | |
| 70. | | | | | |
| 71. | | | | | |
| 72. | | | | | |
| 73. | | | | | |

(Section C, with rows continued)

| | | | | |
|---|---|---|---|---|
| 5. | 60bp: | 83bp: | 61bp: | |
| | gggaagaagggcggcaatga | catggaaataaggaattggggcacaga | aggtagagacaaggagagaag | |
| | gatacagctggggaagaagg | gataagaggtttgggcacagaaataag | gggttggggtacttgccctgtccct | |
| | gcggcaatgagatacagctg | ggattggggcacagagataaggggttg | ggaaaagcagagaag | |
| | (SEQ ID NO: 28) | gg (SEQ ID NO: 29) | (SEQ ID NO: 30) | |
| . . . | | | | |
| 16. | gact | gctata | | |
| . . . | | | | |
| 32. | 61bp: | 3bp: cct | 3bp: gat | 18bp: |
| | gggaggggcaagtatcccaa | | | tatcaacccttaccaca |
| | ccccttctctccgtgtctctaccc | | | a (SEQ ID NO:32) |
| | cttctctgcttttctga | | | |
| | (SEQ ID NO: 31) | | | |
| . . . | | | | |
| 34. | 65bp: | | | |
| | tttcctggggcaggggcaann | | | |
| | nnnnnnnnnnnnnnnnnnc | | | |
| | cttcacccttagccgcaagtccc | | | |
| | gc (SEQ ID NO: 33) | | | |

TABLE 12
from128hervh. (Section A)

| | | | |
|---|---|---|---|
| 1. | hg38 (from 128 LTR7/HERVH | Bonobo failures | Chimp |
| | most active in hESC) | | |
| 2. | chr1: 212910007-212914681 | #Partially deleted in new | #Partially deleted in new |
| 3. | chr1: 55022707-55028369 | #Partially deleted in new | #Partially deleted in new |
| 4. | chr1: 68386003-68391992 | #Partially deleted in new | #Partially deleted in new |
| 5. | chr1: 72987800-72993602 | #Partially deleted in new | #Partially deleted in new |
| 6. | chr1: 81245282-81251207 | #Partially deleted in new | #Partially deleted in new |
| 7. | chr1: 99509510-99515367 | #Partially deleted in new | #Partially deleted in new |
| 8. | chr10: 25768955-25774917 | #Partially deleted in new | #Partially deleted in new |
| 9. | chr10: 54166675-54172501 | #Partially deleted in new | #Partially deleted in new |
| 10. | chr10: 58860994-58867331 | #Partially deleted in new | #Partially deleted in new |
| 11. | chr11: 27629071-27634926 | #Partially deleted in new | #Partially deleted in new |
| 12. | chr12: 14705420-14710640 | #Partially deleted in new | #Partially deleted in new |
| 13. | chr12: 59323187-59328986 | #Partially deleted in new | #Partially deleted in new |
| 14. | chr12: 67766803-67772346 | #Split in new | #Deleted in new |
| 15. | chr13: 51169865-51175006 | #Partially deleted in new | #Partially deleted in new |
| 16. | chr14: 38190637-38196525 | #Partially deleted in new | #Partially deleted in new |
| 17. | chr14: 47104196-47108765 | #Partially deleted in new | #Partially deleted in new |
| 18. | chr16: 13352582-13358061 | #Partially deleted in new | #Partially deleted in new |
| 19. | chr16: 65229804-65235349 | #Partially deleted in new | #Partially deleted in new |
| 20. | chr2: 209299312-209304932 | #Partially deleted in new | #Partially deleted in new |
| 21. | chr2: 64252413-64257646 | #Partially deleted in new | #Partially deleted in new |
| 22. | chr2: 77088246-77094030 | #Partially deleted in new | #Partially deleted in new |
| 23. | chr2: 7872705-7878891 | #Partially deleted in new | #Partially deleted in new |
| 24. | chr20: 40269053-40274761 | #Partially deleted in new | #Partially deleted in new |
| 25. | chr3: 115793482-115799166 | #Partially deleted in new | #Split in new |
| 26. | chr3: 78581211-78588919 | #Partially deleted in new | #Partially deleted in new |
| 27. | chr4: 23722872-23727866 | #Partially deleted in new | #Partially deleted in new |
| 28. | chr4: 24500974-24506750 | #Partially deleted in new | #Partially deleted in new |
| 29. | chr4: 61764217-61770025 | #Partially deleted in new | #Partially deleted in new |
| 30. | chr4: 92271491-92277648 | #Partially deleted in new | #Partially deleted in new |
| 31. | chr5: 106978587-106984086 | #Split in new | #Partially deleted in new |
| 32. | chr5: 120697545-120703411 | #Partially deleted in new | #Partially deleted in new |
| 33. | chr5: 147869835-147874526 | #Partially deleted in new | #Partially deleted in new |
| 34. | chr6: 114422438-114428297 | #Partially deleted in new | #Split in new |
| 35. | chr6: 115031792-115037619 | #Partially deleted in new | #Deleted in new |
| 36. | chr6: 131295356-131301196 | #Partially deleted in new | #Partially deleted in new |
| 37. | chr6: 142015665-142021782 | #Partially deleted in new | #Partially deleted in new |
| 38. | chr9: 87410693-87416706 | #Partially deleted in new | #Partially deleted in new |
| 39. | chr9: 97214493-97220014 | #Partially deleted in new | #Partially deleted in new |
| 40. | chrX: 114466671-114472531 | #Partially deleted in new | #Partially deleted in new |
| 41. | chrX: 4891613-4897331 | #Partially deleted in new | #Split in new |
| 42. | chrX: 92100239-92105917 | #Partially deleted in new | #Partially deleted in new |

(Section B, with rows continued)

| 1. | HERVH- | hg38 to Bonobo | Bonobo LiftOver | hg38 | Direct to Chimp | hg19 reciprocal | |
|---|---|---|---|---|---|---|---|
| | derived | | | reciprocal | | from Chimp | |
| | transcripts | | | from Bonobo | | | |
| 2. | | chr4: 179166475- | JH650542: 6849370- | Partially | #Partially deleted | N/A | |
| | | 179170568 | 6853662 | deleted | in new | | |
| | | | | in new | | | |
| 3. | | chr5: 104063634- | JH650575: 1751459- | Partially | #Split in new | N/A | Deletions in |
| | | 104070481 | 1758624 | deleted | | | both Bonoob |
| | | | | in new | | | and Chimp |
| 4. | | chr5: 122474225- | JH650560: 7443946- | Partially | #Partially deleted | N/A | Deletions in |
| | | 122478846 | 7448815 | deleted | in new | | both Bonoob |
| | | | | in new | | | and Chimp |
| 5. | | chr9: 118485632- | JH650632: 5405353- | Partially | #Partially deleted | N/A | Deletions in |
| | | 118491397 | 5411479 | deleted | in new | | both Bonoob |
| | | | | in new | | | and Chimp |
| 6. | | | | | | | |
| 7. | | hg38 to Chimp | PanTro4 LiftOver | Bonobo | Reciprocal from Chimp to hg19 | | |
| | | | | failure | | | |
| 8. | | chr12: 4018540- | chr12: 4116781- | Partially | Partially deleted in new | | |
| | | 4023694 | 4122861 | deleted | | | |
| | | | | in new | | | |
| 9. | | | Inserts between block 8 and 9 in window | | | | |
| 10. | | | B D Chimp 948bp | | | | |
| 11. | | | 4019658 | 4019659 | | | |
| 12. | | | | | | | |
| 13. | | PanTro4 to hg19 | PanTro4 | hg38 | Bonobo | | Bonobo |
| | | (reciprocal) | | | | | to hg38 |
| | | | | | | | (reciprocal) |
| 14. | | #Partially | chr1: 202294224- | chr1: | JH650419: 502586- | Candidate | #Partially |
| | | deleted in new | 202301010 | 223024395- | 509368 | human- | deleted in |
| | | | | 223030156 | | specific | new |
| 15. | | | Inserts between block 2 and 3 in window | | | | |
| 16. | | | B D Chimp 974bp | | | | |
| 17. | | | B D Bonobo 976bp | | | | |
| 18. | | | 2.23E+08 | 2.23E+08 | | | |
| 19. | | | | | | | |
| 20. | | | Inserts between block 1 and 2 in window | | | | |
| 21. | | | B D Gorilla 7256bp | | | | |
| 22. | | | 2.23E+08 | 2.23E+08 | | | |
| 23. | | | | | | | |
| 24. | | | | | | | |
| 25. | | | | | | | |
| 26. | | | | | | | |
| 27. | | | | | | | |
| 28. | | | | | | | |
| 29. | | | | | | | |
| 30. | | | | | | | |
| 31. | | | | | | | |
| 32. | | | | | | | |
| 33. | | | | | | | |
| 34. | | | | | | | |
| 35. | | | | | | | |
| 36. | | | | | | | |
| 37. | | | | | | | |
| 38. | | | | | | | |
| 39. | | | | | | | |
| 40. | | | | | | | |
| 41. | | | | | | | |
| 42. | | | | | | | |

TABLE 13
HERVH-derived lincRNAs.
(Section A)

| 1. | HERVH-derived lincRNAs | | | | | | | | | |
|---|---|---|---|---|---|---|---|---|---|---|
| 2. | hg38 | gene_name | Note | FC_hESC/EB | Bonobo LiftOver | Reciprocal | Chimp | Reciprocal | HUMAN_SPECIC | HUMAN_SPECIC |
| | | | | | | from Bonobo | | to Chimp | FULL-LENGTH | INTEGRATION SITE |
| | | | | | | to hg38 | | | SEQUENCE | |
| | | | | | | | | | ALIGNMENT | |
| 3. | chr1: 229, 174, 100-229, | MIR4454 | MIR4454 at | 12.01562 | JH650550:528345- | #Partially | #Partially deleted in new | | Bonobo | |
| | 180, 291 | | chr1: 229174683- | | 535499 | deleted in | | | | |
| | | | 229174801- | | | new | | | | |
| | | | (NR_039659) | | | | | | | |
| 4. | chr10: 89, 283, 765-89, | RP11-149123.3 | | 1.132223 | JH650556:210188- | #Partially deleted in new | | | Bonobo | |
| | 292, 125 | | | | 219341 | | | | | |
| 5. | chr11: 3, 469, 319-3, | RP13-726E6.1 | | 1.504637 | #Split in new | | #Split in new | | YES | YES |
| | 486, 328 | | | | | | | | | |
| 6. | chr11: 3, 469, 382-3, | RP13-726E6.2 | | 1.931283 | #Split in new | | #Split in new | | YES | YES |
| | 486, 073 | | | | | | | | | |
| 7. | chr11: 96, 587, 502-96, | RP11-360K13.1 | | 1.890427 | #Partially deleted in new | | #Partially deleted in new | | YES | Gorilla closest alignment |
| | 595, 007 | | | | | | | | | |
| 8. | chr12: 4, 018, 137-4, | RP11-320N7.2 | | 0.631042 | #Partially deleted in new | | chr12: 4116378- | #Partially | Chimp | |
| | 023, 818 | | | | | | 4122985 | deleted in | | |
| | | | | | | | | new | | |
| 9. | chr14: 38, 189, 789-38, | CTD-2142D14.1 | | 4.293359 | #Partially deleted in new | | #Partially deleted in new | | YES | |
| | 196, 600 | | | | | | | | | |
| 10. | chr14: 38, 190, 286-38, | CTD-2058B24.2 | | 2.20354 | #Partially deleted in new | | #Partially deleted in new | | YES | |
| | 197, 000 | | | | | | | | | |
| 11. | chr16: 65, 229, 056-65, | RP11-25619.3 | | 1.106558 | #Partially deleted in new | | #Partially deleted in new | | YES | |
| | 235, 820 | | | | | | | | | |
| 12. | chr16: 65, 229, 500-65, | RP11-25619.2 | | 1.801303 | #Partially deleted in new | | #Partially deleted in new | | YES | |
| | 235, 500 | | | | | | | | | |
| 13. | chr17: 34, 182, 098-34, | RP11-215E13.1 | | 0.300893 | #Split in new | | #Partially deleted in new | | YES | YES |
| | 189, 358 | | | | | | | | | |
| 14. | chr18: 73, 324, 500-73, | CTD-2354A18.1 | | 0.78657 | JH650563:26209175- | #Partially | #Partially deleted in new | | YES | |
| | 330, 500 | | | | 26215407 | deleted in | | | | |
| | | | | | | new | | | | |
| 15. | chr22: 16, 611, 044-16, | TPTEP1 | | 0.48934 | #Split in new | | #Split in new | | YES | YES |
| | 615, 809 | | | | | | | | | |
| 16. | chr4: 132, 117, 632-132, | RP11-78902.1 | | 2.60954 | #Split in new | | #Split in new | | Bonobo | |
| | 124, 853 | | | | | | | | | |
| 17. | chr4:23,722,231-23, | RP11-380P13.2 | ERVH-1 | 3.29528 | #Partially deleted in new | | #Partially deleted in new | | YES | YES |
| | 728, 000 | | | | | | | | | |
| 18. | chr4: 92, 271, 100-92, | RP11-562F9.2 | | 10.64934 | #Partially deleted in new | | #Partially deleted in new | | YES | Bonobo closest alignment |
| | 277, 905 | | | | | | | | | |
| 19. | chr5: 106, 978, 303-106, | CTC-254B4.1 | | 3.272568 | #Split in new | | #Partially deleted in new | | Bonobo | |
| | 984, 967 | | | | | | | | | |
| 20. | chr5: 92, 822, 649-92, | CTC-458G6.4 | | 1.591137 | #Partially deleted in new | | #Partially deleted in new | | YES | Bonobo closest alignment |
| | 829, 398 | | | | | | | | | |
| 21. | chr8: 114, 280, 697-114, | RP11-267L5.1 | | 6.297389 | JH650540:5002141- | #Partially | #Partially deleted in new | | YES | Bonobo closest alignment |
| | 288, 463 | | | | 5017038 | deleted in | | | | |
| | | | | | | new | | | | |
| 22. | chrX: 109, 865, 747-109, | MIR4454 | MIR4454 at | 12.01562 | #Partially deleted in new | | #Partially deleted in new | | YES | Bonobo closest alignment |
| | 870, 946 | | chrX: 109870401- | | | | | | | |
| | | | 109870452- | | | | | | | |
| | | | (NR_039659) | | | | | | | |
| 23. | HERVH-derived lincRNAs | | | | | | | | | |

(Section B, with rows continued)

| 1. | Human-specific deletions of ancestral DNA (size, bp) | | | | |
|---|---|---|---|---|---|
| 2. | Chimp | Bonobo | Gorilla | Orangutan | Gibbon |
| 3. | 68 | 890 | | | |
| 4. | 4,030 | 800 | 3,035 | 12,542 | 12,577 |
| 5. | 5,907 | 5,854 | | | |
| 6. | 5,907 | 5,854 | | | |
| 7. | | | | | |
| 8. | 948 | | | | |
| 9. | | 20 | 2,728 | | |
| 10. | | 20 | 2,728 | | |
| 11. | 4 | 28 | | | 92 |
| 12. | 4 | 28 | | | 92 |
| 13. | 4,046 | 3,162 | | | 833 |
| 14. | | | 4,963 | | |
| 15. | | | | | |
| 16. | 27,843 | 24,411 | 5,650 | | 31,229 |
| 17. | 5,431 | 3,625 | 4,346 | | |
| 18. | 332 | | | | |
| 19. | 10 | | | | |
| 20. | 7,214 | 3,035 | | 6,822 | |
| 21. | 17 | 13 | 5,483 | 616 | 2,298 |
| 22. | | | | | |
| 23. | | | | | |

13 events of distinct deletions compared to genomes of at least 2 different species of non-human primates

TABLE 14
43 human-specific integration sites.
(Section A)

| 1. | 43 human-specific | | | | | | | | | |
|---|---|---|---|---|---|---|---|---|---|---|
| | integration sites | | | | | | | | | |
| 2. | hg38 | Bonobo | Chimp | Expression | HUMAN_ | HUMAN_ | High Confidence | | | |
| | | LiftOver | LiftOver | type | SPECIC | SPECIC | | | | |
| | | | | | SE- | INTEGRA- | | | | |
| | | | | | QUENCE | TION | | | | |
| | | | | | | SITE | | | | |
| 3. | chr14 | #Deleted | #Deleted | highly | YES | YES | YES | | | |
| | 102410503 | in new | in new | active | | | | | | |
| | 102411706 | | | | | | | | | |
| 4. | chr1 | #Partially | #Partially | highly | YES | chr1: 112, | YES | HERVH/ | chr1: | chr1: |
| | 112809666 | deleted | deleted | active | | 821, 143-112, | | AluY/ | 112821 | 112823 |
| | 112826054 | in new | in new | | | 826, 054 | | HERVH/ | 143- | 542- |
| | | | | | | 4,912 bp | | LTR7 | 112822269 | 112825658 |
| 5. | chr2 | #Partially | #Partially | highly | YES | YES | | | | |
| | 77088246 | deleted | deleted | active | | | | | | |
| | 77094030 | in new | in new | | | | | | | |
| 6. | chr4 | #Partially | #Partially | highly | YES | YES | YES | chr4: 61, 757, 766-61, 771, 477 | | |
| | 61764217 | deleted | deleted | active | | | | 13,712 bp. | | |
| | 61770025 | in new | in new | | | | | | | |
| 7. | chr9 | #Partially | #Partially | highly | YES | YES | YES | chr9: 87409190-87418209 9,020 bp | | |
| | 87410693 | deleted | deleted | active | | | | | | |
| | 87416706 | in new | in new | | | | | | | |
| 8. | chr9 | #Partially | #Partially | highly | YES | YES | | | | |
| | 115473180 | deleted | deleted | active | | | | | | |
| | 115478918 | in new | in new | | | | | | | |
| 9. | chr20 | #Partially | #Partially | highly | YES | YES | | | | |
| | 12340266 | deleted | deleted | active | | | | | | |
| | 12345939 | in new | in new | | | | | | | |
| 10. | chrX | #Partially | #Split in | highly | YES | YES | | | | |
| | 114466671 | deleted | new | active | | | | | | |
| | 114472531 | in new | | | | | | | | |
| 11. | chrY | #Partially | #Split in | highly | YES | YES | | | | |
| | 5324786 | deleted | new | active | | | | | | |
| | 5330427 | in new | | | | | | | | |
| 12. | chr1 | #Partially | #Partially | moderately | YES | YES | | | | |
| | 5044795 | deleted | deleted | active | | | | | | |
| | 5053098 | in new | in new | | | | | | | |
| 13. | chr1 | #Partially | #Partially | moderately | YES | YES | chr1: 99508046-99516831 8,786 bp | | | |
| | 99509510 | deleted | deleted | active | | | | | | |
| | 99515367 | in new | in new | | | | | | | |
| 14. | chr10 | #Partially | #Partially | moderately | YES | YES | | | | |
| | 25768955 | deleted | deleted | active | | | | | | |
| | 25774917 | in new | in new | | | | | | | |
| 15. | chr11 | #Split | #Split in | moderately | YES | YES | | | | |
| | 3470256 | in new | new | active | | | | | | |
| | 3485187 | | | | | | | | | |
| 16. | chr11 | #Split | #Split in | moderately | YES | YES | chr11: 71733794-71756475 22,682 bp | | | |
| | 71737574 | in new | new | active | | | | | | |
| | 71752695 | | | | | | | | | |
| 17. | chr14 | #Partially | #Partially | moderately | YES | YES | chr14: 41514368-41523384 9,017 bp. | | | |
| | 41515870 | deleted | deleted | active | | | | | | |
| | 41521881 | in new | in new | | | | | | | |
| 18. | chr19 | #Partially | #Partially | moderately | YES | YES | | | | |
| | 22568269 | deleted | deleted | active | | | | | | |
| | 22575020 | in new | in new | | | | | | | |
| 19. | chr2 | #Deleted | #Partially | moderately | YES | YES | chr2: 57190655-57200305 9,651 bp | | | |
| | 57192262 | in new | deleted | active | | | | | | |
| | 57198696 | | in new | | | | | | | |
| 20. | chr22 | #Partially | #Partially | moderately | YES | YES | chr22: 16608907-16617551 8,645 bp | | | |
| | 16611307 | deleted | deleted | active | | | | | | |
| | 16615149 | in new | in new | | | | | | | |
| 21. | chr4 | #Split | #Split in | moderately | YES | YES | | | | |
| | 3927445 | in new | new | active | | | | | | |
| | 3933080 | | | | | | | | | |
| 22. | chr5 | #Deleted | #Deleted | moderately | YES | YES | YES | chr5: 12489144-12495547 6,404 bp | | |
| | 12490211 | in new | in new | active | | | | | | |
| | 12494480 | | | | | | | | | |
| 23. | chr8 | #Deleted | #Partially | moderately | YES | YES | YES | chr8: 104, 284, 367-104, 293, 639 | | |
| | 104285911 | in new | deleted | active | | | | 9,273 bp | | |
| | 104292093 | | in new | | | | | | | |
| 24. | chr8 | #Partially | #Partially | moderately | YES | YES | chr8: 144, 952, 399-144, 961, 518 9,120 bp. | | | |
| | 144953918 | deleted | deleted | active | | | | | | |
| | 144959998 | in new | in new | | | | | | | |
| 25. | chrX | #Partially | #Partially | moderately | YES | YES | chrX: 119, 316, 348-119, 324, 896 8,549 bp | | | |
| | 119317772 | deleted | deleted | active | | | | | | |
| | 119323471 | in new | in new | | | | | | | |
| 26. | chr1 | #Deleted | #Deleted | Inactive | YES | YES | YES | truncated LTR7/HERVH next to | | |
| | 84058413 | in new | in new | | | | | L1HS | | |
| | 84058945 | | | | | | | | | |
| 27. | chr10 | #Partially | #Deleted | Inactive | YES | YES | YES | truncated LTR7/HERVH next | | |
| | 17630036 | deleted | in new | | | | | to SVA_F | | |
| | 17632161 | in new | | | | | | | | |
| 28. | chr11 | #Split | #Split in | Inactive | YES | YES | YES | | | |
| | 4315701 | in new | new | | | | | | | |
| | 4321901 | | | | | | | | | |
| 29. | chr12 | #Partially | #Partially | Inactive | YES | YES | | | | |
| | 25163212 | deleted | deleted | | | | | | | |
| | 25169515 | in new | in new | | | | | | | |
| 30. | chr19 | #Deleted | #Partially | Inactive | YES | YES | YES | | | |
| | 38750365 | in new | deleted | | | | | | | |
| | 38755295 | | in new | | | | | | | |
| 31. | chr2 | #Partially | #Partially | Inactive | YES | YES | | | | |
| | 3815548 | deleted | deleted | | | | | | | |
| | 3821340 | in new | in new | | | | | | | |
| 32. | chr2 | #Partially | #Partially | Inactive | YES | YES | YES | SVA_D human-specific insert within | | |
| | 71157777 | deleted | deleted | | | | | LTR7/HERVH | | |
| | 71165609 | in new | in new | | | | | | | |
| 33. | chr4 | #Partially | #Partially | Inactive | YES | YES | YES | | | |
| | 115975699 | deleted | deleted | | | | | | | |
| | 115981223 | in new | in new | | | | | | | |
| 34. | chr4 | #Partially | #Partially | Inactive | YES | YES | YES | LTR12C insert within LTR7/HERVH | | |
| | 27974888 | deleted | deleted | | | | | | | |
| | 27981374 | in new | in new | | | | | | | |
| 35. | chr4 | #Split | #Split | Inactive | YES | YES | YES | HERVE/LTR2C insert within | | |
| | 9094399 | in new | in new | | | | | LTR7/HERVH | | |
| | 9108459 | | | | | | | | | |
| 36. | chr6 | #Partially | #Partially | Inactive | YES | YES | | L1PA3 insert within LTR7Y/HERVH | | |
| | 81343927 | deleted | deleted | | | | | | | |
| | 81351160 | in new | in new | | | | | | | |
| 37. | chr9 | #Deleted | #Deleted | Inactive | YES | YES | YES | Truncared LTR7Y/HERVH | | |
| | 86586833 | in new | in new | | | | | | | |
| | 86589057 | | | | | | | | | |
| 38. | chrX | #Partially | #Deleted | Inactive | YES | YES | YES | | | |
| | 64651095 | deleted | in new | | | | | | | |
| | 64657665 | in new | | | | | | | | |
| 39. | chrY | #Deleted | #Deleted | Inactive | YES | YES | YES | | | |
| | 10047167 | in new | in new | | | | | | | |
| | 10053754 | | | | | | | | | |
| 40. | chrY | #Split | #Split in | Inactive | YES | YES (probable) | | truncated HERV9 next to | | |
| | 15769836 | in new | new | | | | | HERVH/LTR7; LTR5_Hs nearby | | |
| | 15773029 | | | | | | | | | |
| 41. | chrY | #Deleted | #Deleted | Inactive | YES | YES | YES | Several adjacent copies of | | |
| | 21035919 | in new | in new | | | | | LTR7/HERVH | | |
| | 21045245 | | | | | | | | | |
| 42. | | | | | | | | | | |
| 43. | | | | | | 39 human-specific integration sites | | | | |
| 44. | | | | | | 4 additional sites with other repeats involved | | | | |
| 45. | | | | | | | | | | |
| 46. | chr10 | #Partial | #Deleted | Inactive | YES | LiHS | YES | L1HS human-specific insert within | | |
| | 79963907 | deleted | in new | | | sequence | | LTR7/HERVH | | |
| | 79968032 | in new | | | | insert | | | | |
| 47. | chr11 | #Partially | #Partially | Inactive | YES | L1PA2 | YES | L1PA2 human-specific insert within | | |
| | 122824427 | deleted | deleted | | | sequence | | LTR7/HERVH | | |
| | 122832822 | in new | in new | | | insert | | | | |
| 48. | chr11 | #Split | #Split in | Inactive | YES | LTR2C/ | YES | LTR2C/HERVE human-specific insert | | |
| | 67841905 | in new | new | | | HERVE | | within LTR7/HERVH | | |
| | 67856961 | | | | | sequence | | | | |
| | | | | | | insert | | | | |
| 49. | chr14_ | #Partially | #Partially | Inactive | YES | chr1_ | YES | truncated HERVH next to | | |
| | GL000009v2_ | deleted | deleted | | | GL000009v2_ | | human-specific SVA_D insert | | |
| | random | in new | in new | | | random: | | | | |
| | 197844 | | | | | 199, 076-201, | | | | |
| | 199392 | | | | | 397 | | | | |
| | | | | | | 2,322 bp. | | | | |

(Section B, with rows continued)

| 1. | Human-specific deletions of ancestral DNA (size, bp) | | | | |
|---|---|---|---|---|---|
| 2. | Chimp | Bonobo | Gorilla | Orangutan | Gibbon |
| 3. | | | | | 1,190 |
| 4. | 7,433 | 4 | 6,995; 4655 | | 71,036 |
| 5. | 4,462 | 5,110 | | | |
| 6. | 7,599 | | | 13,298 | 143 |
| 7. | 4 | 4 | 4 | 4; 5 | |
| 8. | 5,679 | | 5,858 | | |
| 9. | | 3,391 | | | |
| 10. | 9,025 | 3,148 | 5,927 | | |
| 11. | | 4,676 | 9,965 | | |
| 12. | 13,562 | 14,752 | 17,588 | 8,519 | 9,799 |
| 13. | | 5,036 | | | |
| 14. | | 2,775 | | | |
| 15. | 5,907 | 5,854 | | | |
| 16. | 2,841 | 16,377; | 13,109 | 2 | 523 |
| | | 11,640 | | | |
| 17. | 3,175 | | 6,977 | | |
| 18. | 5,907 | | 12,891 | | |
| 19. | 5,376 | 5,184 | 100 | 2 | 9 |
| 20. | | 330 | 407 | | |
| 21. | | 81,108 | | 35,326 | |
| 22. | | | 2,637 | 1,255 | |
| 23. | 8,318 | | 3,998 | 595 | 5 |
| 24. | | | 9,101 | 1,875 | 3,228 |
| 25. | | | 4,180 | | |
| 26. | | | | | |
| 27. | | | | | |
| 28. | | | | 5,753 | |
| 29. | | 10,245 | | | |
| 30. | 6,565 | | | | |
| 31. | | | | | |
| 32. | 19 | 3,298 | | | |
| 33. | | | 5,797 | 53 | 686 |
| 34. | | | | | |
| 35. | | | | | |
| 36. | 4,827 | | | | |
| 37. | | | | | |
| 38. | | | | | |
| 39. | | | | | |
| 40. | | | | | |
| 41. | | | | | |
| 42. | | | | | |
| 43. | | | | | |
| 44. | | | | | |
| 45. | | | | | |
| 46. | | | 1,567 | | 3,498 |
| 47. | | | 8,685 | | 288 |
| 48. | 1,150 | | | | |
| 49. | | | | | |

TABLE 15
SNMs10datasets.
(Section A)

| | | | | | | 19 cohorts | |
|---|---|---|---|---|---|---|---|
| | | | | | | Pancancer19 | |
| | SNMs Pancancer | | Poor | | | Percent in poor | P value Somatic |
| 1. | awe 19 cohorts | Gene | prognosis | Good prognosis | | prognosis group | non-silent mutations |
| 2. | 4,429 samples | TP53 | 1517 | 2715 | 4232 | 35.8 | 1.42E−11 |
| 3. | | PCDH15 | 268 | 3964 | 4232 | 6.3 | 0.0133 |
| 4. | | DMD | 254 | 3978 | 4232 | 6.0 | 0.88 |
| 5. | | NF1 | 214 | 4018 | 4232 | 5.1 | 0.015 |
| 6. | | NOTCH1 | 144 | 4088 | 4232 | 3.4 | 0.013 |
| 7. | | EGFR | 185 | 4047 | 4232 | 4.4 | 0.00E+00 |
| 8. | | MALAT1 | 152 | 4080 | 4232 | 3.6 | 0.011 |
| 9. | | RB1 | 132 | 4100 | 4232 | 3.1 | 0.85 |
| 10. | | LPHN3 | 125 | 4107 | 4232 | 3.0 | 0.65 |
| 11. | | KDM6A | 90 | 4142 | 4232 | 2.1 | 0.58 |
| 12. | | TLR4 | 105 | 4127 | 4232 | 2.5 | 0.22 |
| 13. | | KEAP1 | 90 | 4142 | 4232 | 2.1 | 0.12 |
| 14. | | SMAD4 | 74 | 4158 | 4232 | 1.7 | 0.034 |
| 15. | | PRX | 72 | 4160 | 4232 | 1.7 | 0.21 |
| 16. | | EPHA7 | 90 | 4142 | 4232 | 2.1 | 0.38 |
| 17. | | IDH1 | 198 | 4034 | 4232 | 4.7 | 0.12 |
| 18. | | KIAA1244 | 69 | 4163 | 4232 | 1.6 | 0.99 |
| 19. | | STK11 | 35 | 4197 | 4232 | 0.8 | 0.013 |
| 20. | | PTPN11 | 49 | 4183 | 4232 | 1.2 | 0.11 |
| 21. | | ELF3 | 33 | 4199 | 4232 | 0.8 | 0.81 |
| 22. | | VEZF1 | 28 | 4204 | 4232 | 0.7 | 0.12 |
| 23. | | DAB2IP | 45 | 4187 | 4232 | 1.1 | 0.0084 |
| 24. | | GLUD2 | 45 | 4187 | 4232 | 1.1 | 0.39 |
| 25. | | ZNF28 | 39 | 4193 | 4232 | 0.9 | 0.24 |
| 26. | | DPPA2 | 42 | 4190 | 4232 | 1.0 | 0.054 |
| 27. | | CHST6 | 27 | 4205 | 4232 | 0.6 | 0.22 |
| 28. | | FEZ2 | 9 | 4223 | 4232 | 0.2 | 0.26 |
| 29. | | KRAS | 249 | 3983 | 4232 | 5.9 | 1 |
| 30. | | CDKN2A | 161 | 4071 | 4232 | 3.8 | 0.015 |
| 31. | | DNMT3A | 114 | 4118 | 4232 | 2.69376 | 3.42E−07 |
| 32. | | FLT3 | 124 | 4108 | 4232 | 2.93006 | 0.001 |
| 33. | | NFE2L2 | 88 | 4144 | 4232 | 2.0794 | 0.15 |
| 34. | | NPM1 | 65 | 4167 | 4232 | 1.53592 | 6.48E−11 |
| 35. | | MIR142 | 6 | 4226 | 4232 | 0.14178 | 0.3 |
| 36. | | FOXL2 | 7 | 4225 | 4232 | 0.16541 | 0.0058 |
| 37. | | H3F3A | 10 | 4222 | 4232 | 0.23629 | 0.97 |
| 38. | | H3F3B | 11 | 4221 | 4232 | 0.25992 | 0.1 |
| 39. | | KMT2D | ND | | | | |
| 40. | | RNF43 | 53 | 4179 | 4232 | 1.25236 | 0.7 |
| 41. | | TERT | 37 | 4195 | 4232 | 0.87429 | 0.0021 |
| 42. | | ERBB2 | 72 | 4160 | 4232 | 1.70132 | 0.57 |
| 43. | | PLCG1 | 62 | 4170 | 4232 | 1.46503 | 0.67 |

(Section B, with rows continued)

| | | | | | | Xena-1 | |
|---|---|---|---|---|---|---|---|
| | | | | | | Pancancer29 | |
| | | | Poor | | | Percent in poor | P value Somatic non- |
| 1. | Xena-1 | Gene | prognosis | Good prognosis | | prognosis group | silent mutations |
| 2. | 7509 samples | TP53 | 2630 | 4445 | 7075 | 37.2 | 0.00E+00 |
| 3. | | PCDH15 | 515 | 6560 | 7075 | 7.3 | 2.77E−05 |
| 4. | | DMD | 465 | 6610 | 7075 | 6.6 | 0.031 |
| 5. | | NF1 | 394 | 6681 | 7075 | 5.6 | 3.93E−06 |
| 6. | | NOTCH1 | 298 | 6777 | 7075 | 4.2 | 0.016 |
| 7. | | EGFR | 293 | 6782 | 7075 | 4.1 | 0.00E+00 |
| 8. | | MALAT1 | 277 | 6798 | 7075 | 3.9 | 0.00043 |
| 9. | | RB1 | 276 | 6799 | 7075 | 3.9 | 0.00059 |
| 10. | | LPHN3 | 242 | 6833 | 7075 | 3.4 | 0.0094 |
| 11. | | KDM6A | 223 | 6852 | 7075 | 3.2 | 9.93E−05 |
| 12. | | TLR4 | 192 | 6883 | 7075 | 2.7 | 0.031 |
| 13. | | KEAP1 | 185 | 6890 | 7075 | 2.6 | 0.00011 |
| 14. | | SMAD4 | 177 | 6898 | 7075 | 2.5 | 2.58E−08 |
| 15. | | PRX | 154 | 6921 | 7075 | 2.2 | 0.01 |
| 16. | | EPHA7 | 158 | 6917 | 7075 | 2.2 | 2.53E−05 |
| 17. | | IDH1 | 486 | 6589 | 7075 | 6.9 | 0.0015 |
| 18. | | KIAA1244 | 149 | 6926 | 7075 | 2.1 | 0.0064 |
| 19. | | STK11 | 114 | 6961 | 7075 | 1.6 | 0.00011 |
| 20. | | PTPN11 | 63 | 7012 | 7075 | 0.9 | 0.00023 |
| 21. | | ELF3 | 96 | 6979 | 7075 | 1.4 | 0.02 |
| 22. | | VEZF1 | 77 | 6998 | 7075 | 1.1 | 0.019 |
| 23. | | DAB2IP | 96 | 6979 | 7075 | 1.4 | 4.21E−05 |
| 24. | | GLUD2 | 91 | 6984 | 7075 | 1.3 | 0.024 |
| 25. | | ZNF28 | 82 | 6993 | 7075 | 1.2 | 0.012 |
| 26. | | DPPA2 | 74 | 7001 | 7075 | 1.0 | 0.032 |
| 27. | | CHST6 | 52 | 7023 | 7075 | 0.7 | 0.039 |
| 28. | | FEZ2 | 30 | 7045 | 7075 | 0.4 | 0.014 |
| 29. | | KRAS | | | | | NS |
| 30. | | CDKN2A | | | | | NS |
| 31. | | DNMT3A | | | | | NS |
| 32. | | FLT3 | | | | | NS |
| 33. | | NFE2L2 | | | | | NS |
| 34. | | NPM1 | | | | | NS |
| 35. | | MIR142 | | | | | NS |
| 36. | | FOXL2 | | | | | ND |
| 37. | | H3F3A | | | | | ND |
| 38. | | H3F3B | | | | | ND |
| 39. | | KMT2D | | | | | ND |
| 40. | | RNF43 | | | | | ND |
| 41. | | TERT | | | | | ND |
| 42. | | ERBB2 | | | | | ND |
| 43. | | PLCG1 | | | | | ND |

(Section C, with rows continued)

| | | | | | | Xena-2 | |
|---|---|---|---|---|---|---|---|
| | | | | | | Pancancer29 | |
| | | | | | | Percent in | |
| | Xena-2 | | | | | poor | P value Somatic |
| | (10.30.2015 | | Poor | | | prognosis | non-silent |
| 1. | version) | Gene | prognosis | Good prognosis | | group | mutations |
| 2. | 7173 samples | TP53 | 1509 | 5436 | 6945 | 21.7 | 1.37E−06 |
| 3. | | PCDH15 | 207 | 6738 | 6945 | 3.0 | 0.42 |
| 4. | | DMD | 274 | 6671 | 6945 | 3.9 | 0.6 |
| 5. | | NF1 | 186 | 6759 | 6945 | 2.7 | 0.016 |
| 6. | | NOTCH1 | 114 | 6831 | 6945 | 1.6 | 0.99 |
| 7. | | EGFR | 151 | 6794 | 6945 | 2.2 | 0.00E+00 |
| 8. | | MALAT1 | 69 | 6876 | 6945 | 1.0 | 0.81 |
| 9. | | RB1 | 124 | 6821 | 6945 | 1.8 | 0.71 |
| 10. | | LPHN3 | 102 | 6843 | 6945 | 1.5 | 0.3 |
| 11. | | KDM6A | 104 | 6841 | 6945 | 1.5 | 0.28 |
| 12. | | TLR4 | 73 | 6872 | 6945 | 1.1 | 0.97 |
| 13. | | KEAP1 | 55 | 6890 | 6945 | 0.8 | 0.93 |
| 14. | | SMAD4 | 133 | 6812 | 6945 | 1.9 | 0.00069 |
| 15. | | PRX | 64 | 6881 | 6945 | 0.9 | 0.67 |
| 16. | | EPHA7 | 63 | 6882 | 6945 | 0.9 | 0.48 |
| 17. | | IDH1 | 426 | 6519 | 6945 | 6.1 | 5.45E−05 |
| 18. | | KIAA1244 | 82 | 6863 | 6945 | 1.2 | 1 |
| 19. | | STK11 | 37 | 6908 | 6945 | 0.5 | 0.0028 |
| 20. | | PTPN11 | 49 | 6896 | 6945 | 0.7 | 0.43 |
| 21. | | ELF3 | 40 | 6905 | 6945 | 0.6 | 0.52 |
| 22. | | VEZF1 | 35 | 6910 | 6945 | 0.5 | 0.33 |
| 23. | | DAB2IP | 44 | 6901 | 6945 | 0.6 | 0.89 |
| 24. | | GLUD2 | 55 | 6890 | 6945 | 0.8 | 0.3 |
| 25. | | ZNF28 | 32 | 6913 | 6945 | 0.5 | 0.59 |
| 26. | | DPPA2 | 28 | 6917 | 6945 | 0.4 | 0.14 |
| 27. | | CHST6 | 34 | 6911 | 6945 | 0.5 | 0.22 |
| 28. | | FEZ2 | 20 | 6925 | 6945 | 0.3 | 0.91 |
| 29. | | KRAS | 386 | 6559 | 6945 | 5.6 | 0.001 |
| 30. | | CDKN2A | 101 | 6844 | 6945 | 1.5 | 6.84E−11 |
| 31. | | DNMT3A | | | | | NS |
| 32. | | FLT3 | | | | | NS |
| 33. | | NFE2L2 | | | | | NS |
| 34. | | NPM1 | | | | | NS |
| 35. | | MIR142 | | | | | NS |
| 36. | | FOXL2 | | | | | ND |
| 37. | | H3F3A | | | | | ND |
| 38. | | H3F3B | | | | | ND |
| 39. | | KMT2D | | | | | ND |
| 40. | | RNF43 | | | | | ND |
| 41. | | TERT | | | | | ND |
| 42. | | ERBB2 | | | | | ND |
| 43. | | PLCG1 | | | | | ND |

(Section D, with rows continued)

| | | | | | | Broad Percent in | | |
|---|---|---|---|---|---|---|---|---|
| | | | Poor | | | poor prognosis | P value Somatic non-silent | |
| 1. | BROAD | Gene | prognosis | Good prognosis | | group | mutations | |
| 2. | 4333 samples | TP53 | 489 | 3739 | 4228 | 11.6 | 2.56E−06 | |
| 3. | | PCDH15 | 62 | 4166 | 4228 | 1.5 | 0.65 | |
| 4. | | DMD | 91 | 4137 | 4228 | 2.2 | 0.83 | |
| 5. | | NF1 | 86 | 4142 | 4228 | 2.0 | 0.00069 | |
| 6. | | NOTCH1 | 32 | 4196 | 4228 | 0.8 | 0.57 | |
| 7. | | EGFR | 90 | 4138 | 4228 | 2.1 | 0.00E+00 | |
| 8. | | MALAT1 | 27 | 4201 | 4228 | 0.6 | 0.87 | |
| 9. | | RB1 | 45 | 4163 | 4208 | 1.1 | 0.037 | |
| 10. | | LPHN3 | 26 | 4202 | 4228 | 0.6 | 0.057 | |
| 11. | | KDM6A | 42 | 4186 | 4228 | 1.0 | 0.55 | |
| 12. | | TLR4 | 16 | 4212 | 4228 | 0.4 | 0.32 | |
| 13. | | KEAP1 | 27 | 4201 | 4228 | 0.6 | 0.66 | |
| 14. | | SMAD4 | 8 | 4220 | 4228 | 0.2 | 0.19 | |
| 15. | | PRX | 11 | 4217 | 4228 | 0.3 | 0.65 | |
| 16. | | EPHA7 | 17 | 4211 | 4228 | 0.4 | 0.71 | |
| 17. | | IDH1 | 21 | 4207 | 4228 | 0.5 | 0.48 | |
| 18. | | KIAA1244 | 19 | 4209 | 4228 | 0.4 | 0.65 | |
| 19. | | STK11 | 21 | 4207 | 4228 | 0.5 | 0.23 | |
| 20. | | PTPN11 | 11 | 4217 | 4228 | 0.3 | 0.025 | |
| 21. | | ELF3 | 4 | 4224 | 4228 | 0.1 | 0.77 | |
| 22. | | VEZF1 | 9 | 4219 | 4228 | 0.2 | 0.84 | |
| 23. | | DAB2IP | 6 | 4222 | 4228 | 0.1 | 0.19 | |
| 24. | | GLUD2 | 20 | 4208 | 4228 | 0.5 | 0.27 | |
| 25. | | ZNF28 | 10 | 4118 | 4128 | 0.2 | 4.33E−06 | |
| 26. | | DPPA2 | 11 | 4217 | 4228 | 0.3 | 0.18 | |
| 27. | | CHST6 | 9 | 4219 | 4228 | 0.2 | 0.19 | |
| 28. | | FEZ2 | 5 | 4223 | 4228 | 0.1 | 0.99 | |
| 29. | | KRAS | 55 | 4173 | 4228 | 1.3 | 0.023 | Good |
| 30. | | CDKN2A | 48 | 4180 | 4228 | 1.1 | 2.32E−05 | |
| 31. | | DNMT3A | 104 | 5715 | 5819 | 1.78725 | 0.00017 | Good |
| 32. | | FLT3 | 105 | 5714 | 5819 | 1.80443 | 0.15 | |
| 33. | | NFE2L2 | 150 | 5669 | 5819 | 2.57776 | 1.60E−09 | |
| 34. | | NPM1 | 17 | 5802 | 5819 | 0.29215 | 0.2 | |
| 35. | | MIR142 | NO DATA | | | | | |
| 36. | | FOXL2 | 13 | 5806 | 5819 | 0.22341 | 0.034 | |
| 37. | | H3F3A | 12 | 5807 | 5819 | 0.20622 | 0.018 | |
| 38. | | H3F3B | 24 | 5795 | 5819 | 0.41244 | 0.015 | |
| 39. | | KMT2D | 423 | 5396 | 5819 | 7.26929 | 0.029 | |
| 40. | | RNF43 | 96 | 5720 | 5816 | 1.65062 | 0.6 | |
| 41. | | TERT | 56 | 5763 | 5819 | 0.96236 | 0.012 | |
| 42. | | ERBB2 | 122 | 5697 | 5819 | 2.09658 | 0.12 | |
| 43. | | PLCG1 | 80 | 5739 | 5819 | 1.37481 | 0.0057 | |

(Section E, with rows continued)

| | | | Poor | | | Pancancer | P value Somatic |
|---|---|---|---|---|---|---|---|
| 1. | UCSC automated vcf | Gene | prognosis | Good prognosis | | UCSC | non-silent mutations |
| 2. | 2970 samples | TP53 | 704 | 2203 | 2907 | 24.2 | 7.13E−12 |
| 3. | | PCDH15 | 206 | 2701 | 2907 | 7.1 | 0.22 |
| 4. | | DMD | 194 | 2713 | 2907 | 6.7 | 0.046 |
| 5. | | NF1 | 121 | 2786 | 2907 | 4.2 | 0.0031 |
| 6. | | NOTCH1 | 126 | 2781 | 2907 | 4.3 | 0.77 |
| 7. | | EGFR | 99 | 2808 | 2907 | 3.4 | 0.0078 |
| 8. | | MALAT1 | 124 | 2783 | 2907 | 4.3 | 0.27 |
| 9. | | RB1 | 52 | 2855 | 2907 | 1.8 | 0.58 |
| 10. | | LPHN3 | 80 | 2827 | 2907 | 2.8 | 0.024 |
| 11. | | KDM6A | 62 | 2845 | 2907 | 2.1 | 0.091 |
| 12. | | TLR4 | 76 | 2831 | 2907 | 2.6 | 0.11 |
| 13. | | KEAP1 | 49 | 2858 | 2907 | 1.7 | 0.31 |
| 14. | | SMAD4 | 68 | 2839 | 2907 | 2.3 | 0.00012 |
| 15. | | PRX | 69 | 2838 | 2907 | 2.4 | 0.76 |
| 16. | | EPHA7 | 85 | 2822 | 2907 | 2.9 | 0.015 |
| 17. | | IDH1 | 424 | 2483 | 2907 | 14.6 | 5.28E−05 |
| 18. | | KIAA1244 | 88 | 2819 | 2907 | 3.0 | 0.093 |
| 19. | | STK11 | 27 | 2880 | 2907 | 0.9 | 0.81 |
| 20. | | PTPN11 | 60 | 2847 | 2907 | 2.1 | 0.46 |
| 21. | | ELF3 | 25 | 2882 | 2907 | 0.9 | 0.41 |
| 22. | | VEZF1 | 10 | 2897 | 2907 | 0.3 | 0.68 |
| 23. | | DAB2IP | 54 | 2853 | 2907 | 1.9 | 0.5 |
| 24. | | GLUD2 | 43 | 2864 | 2907 | 1.5 | 0.43 |
| 25. | | ZNF28 | 31 | 2876 | 2907 | 1.1 | 0.49 |
| 26. | | DPPA2 | 34 | 2873 | 2907 | 1.2 | 0.7 |
| 27. | | CHST6 | 18 | 2889 | 2907 | 0.6 | 0.67 |
| 28. | | FEZ2 | 8 | 2899 | 2907 | 0.3 | 0.53 |
| 29. | | KRAS | 174 | 2733 | 2907 | 6.0 | 1.11E−16 |
| 30. | | CDKN2A | 48 | 2859 | 2907 | 1.7 | 0.074 |
| 31. | | DNMT3A | 61 | 2846 | 2907 | 2.09838 | 0.11 |
| 32. | | FLT3 | 69 | 2838 | 2907 | 2.37358 | 0.63 |
| 33. | | NFE2L2 | 55 | 2852 | 2907 | 1.89198 | 0.97 |
| 34. | | NPM1 | 18 | 2889 | 2907 | 0.6192 | 0.22 |
| 35. | | MIR142 | 3 | 2904 | 2907 | 0.1032 | 0.25 |
| 36. | | FOXL2 | 5 | 2902 | 2907 | 0.172 | 0.055 |
| 37. | | H3F3A | 9 | 2898 | 2907 | 0.3096 | 0.31 |
| 38. | | H3F3B | 7 | 2900 | 2907 | 0.2408 | 0.43 |
| 39. | | KMT2D | 214 | 2693 | 2907 | 7.36154 | 0.25 |
| 40. | | RNF43 | 51 | 2856 | 2907 | 1.75439 | 0.11 |
| 41. | | TERT | 40 | 2867 | 2907 | 1.37599 | 0.19 |
| 42. | | ERBB2 | 86 | 2821 | 2907 | 2.95838 | 0.0059 |
| 43. | | PLCG1 | 65 | 2842 | 2907 | 2.23598 | 0.12 |

(Section F, with rows continued)

| | | | | | | ICGC Pancancer in | P value Somatic |
|---|---|---|---|---|---|---|---|
| | | | Poor | | | poor prognosis | non-silent |
| 1. | ICGC Pancancer | Gene | prognosis | Good prognosis | | group | mutations |
| 2. | 3453 samples | TP53 | 957 | 1581 | 2538 | 37.7 | 0.00E+00 |
| 3. | | PCDH15 | 84 | 2454 | 2538 | 3.3 | 0.31 |
| 4. | | DMD | 59 | 2479 | 2538 | 2.3 | 0.13 |
| 5. | | NF1 | 56 | 2482 | 2538 | 2.2 | 0.36 |
| 6. | | NOTCH1 | 52 | 2486 | 2538 | 2.0 | 0.51 |
| 7. | | EGFR | 13 | 2525 | 2538 | 0.5 | 0.16 |
| 8. | | MALAT1 | 65 | 2473 | 2538 | 2.6 | 0.63 |
| 9. | | RB1 | 44 | 2494 | 2538 | 1.7 | 0.13 |
| 10. | | LPHN3 | 53 | 2334 | 2387 | 2.2 | 0.28 |
| 11. | | KDM6A | 46 | 2492 | 2538 | 1.8 | 0.11 |
| 12. | | TLR4 | 19 | 2519 | 2538 | 0.7 | 0.029 |
| 13. | | KEAP1 | 27 | 2511 | 2538 | 1.1 | 0.96 |
| 14. | | SMAD4 | 160 | 2378 | 2538 | 6.3 | 2.22E−15 |
| 15. | | PRX | 26 | 2512 | 2538 | 1.0 | 0.047 |
| 16. | | EPHA7 | 48 | 2490 | 2538 | 1.9 | 0.92 |
| 17. | | IDH1 | 20 | 2518 | 2538 | 0.8 | 0.11 |
| 18. | | KIAA1244 | 25 | 2171 | 2196 | 1.1 | 0.05 |
| 19. | | STK11 | 6 | 2532 | 2538 | 0.2 | 1.15E−05 |
| 20. | | PTPN11 | 16 | 2522 | 2538 | 0.6 | 0.35 |
| 21. | | ELF3 | 14 | 2524 | 2538 | 0.6 | 0.26 |
| 22. | | VEZF1 | 3 | 2535 | 2538 | 0.1 | 0.95 |
| 23. | | DAB2IP | 8 | 2530 | 2538 | 0.3 | 0.72 |
| 24. | | GLUD2 | 9 | 2529 | 2538 | 0.4 | 0.97 |
| 25. | | ZNF28 | 9 | 2529 | 2538 | 0.4 | 0.17 |
| 26. | | DPPA2 | 4 | 2534 | 2538 | 0.2 | 0.36 |
| 27. | | CHST6 | 12 | 2526 | 2538 | 0.5 | 0.31 |
| 28. | | FEZ2 | 5 | 2533 | 2538 | 0.2 | 0.46 |
| 29. | | KRAS | 589 | 1949 | 2538 | 23.2 | 0.00E+00 |
| 30. | | CDKN2A | 140 | 2398 | 2538 | 5.5 | 2.33E−12 |
| 31. | | DNMT3A | 17 | 2521 | 2538 | 0.66982 | 0.87 |
| 32. | | FLT3 | 18 | 2520 | 2538 | 0.70922 | 0.71 |
| 33. | | NFE2L2 | 42 | 2496 | 2538 | 1.65485 | 0.29 |
| 34. | | NPM1 | 7 | 2531 | 2538 | 0.27581 | 0.096 |
| 35. | | MIR142 | 7 | 2531 | 2538 | 0.27581 | 0.18 |
| 36. | | FOXL2 | 3 | 2535 | 2538 | 0.1182 | 0.81 |
| 37. | | H3F3A | 3 | 2535 | 2538 | 0.1182 | 0.29 |
| 38. | | H3F3B | 5 | 2533 | 2538 | 0.19701 | 0.46 |
| 39. | | KMT2D | 108 | 2430 | 2538 | 4.25532 | 0.17 |
| 40. | | RNF43 | 45 | 2493 | 2538 | 1.77305 | 0.00072 |
| 41. | | TERT | 15 | 2523 | 2538 | 0.59102 | 0.8 |
| 42. | | ERBB2 | 21 | 2517 | 2538 | 0.82742 | 0.19 |
| 43. | | PLCG1 | 17 | 2521 | 2538 | 0.66982 | 0.78 |

(Section G, with rows continued)

| | | | | | | Pancancer | | |
|---|---|---|---|---|---|---|---|---|
| | | | | | | 12 cohorts | | |
| | | | | | | Percent in | | |
| | | | | | | poor | P value Somatic | |
| | | | Poor | | | prognosis | non-silent | |
| 1. | Pancancer12 | Gene | prognosis | Good prognosis | | group | mutations | |
| 2. | 3276 samples | TP53 | 1316 | 1830 | 3146 | 41.8 | 0.0002 | |
| 3. | | PCDH15 | 162 | 2984 | 3146 | 5.1 | 0.99 | |
| 4. | | DMD | 202 | 2944 | 3146 | 6.4 | 0.44 | |
| 5. | | NF1 | 155 | 2991 | 3146 | 4.9 | 0.27 | |
| 6. | | NOTCH1 | 105 | 3041 | 3146 | 3.3 | 0.067 | |
| 7. | | EGFR | 153 | 2993 | 3146 | 4.9 | 0.00E+00 | |
| 8. | | MALAT1 | 87 | 3059 | 3146 | 2.8 | 0.002 | |
| 9. | | RB1 | 114 | 3032 | 3146 | 3.6 | 0.73 | |
| 10. | | LPHN3 | 93 | 3053 | 3146 | 3.0 | 0.48 | |
| 11. | | KDM6A | 74 | 3072 | 3146 | 2.4 | 0.44 | |
| 12. | | TLR4 | 70 | 3076 | 3146 | 2.2 | 0.88 | |
| 13. | | KEAP1 | 80 | 3066 | 3146 | 2.5 | 0.23 | |
| 14. | | SMAD4 | 56 | 3096 | 3152 | 1.8 | 0.92 | |
| 15. | | PRX | 40 | 3106 | 3146 | 1.3 | 0.87 | |
| 16. | | EPHA7 | 60 | 3086 | 3146 | 1.9 | 0.74 | |
| 17. | | IDH1 | 52 | 3094 | 3146 | 1.7 | 0.91 | |
| 18. | | KIAA1244 | 42 | 3104 | 3146 | 1.3 | 0.85 | |
| 19. | | STK11 | 28 | 3118 | 3146 | 0.9 | 0.011 | |
| 20. | | PTPN11 | 33 | 3113 | 3146 | 1.0 | 0.36 | |
| 21. | | ELF3 | 22 | 3124 | 3146 | 0.7 | 0.95 | |
| 22. | | VEZF1 | 19 | 3127 | 3146 | 0.6 | 0.23 | |
| 23. | | DAB2IP | 26 | 3120 | 3146 | 0.8 | 0.26 | |
| 24. | | GLUD2 | 36 | 3110 | 3146 | 1.1 | 0.7 | |
| 25. | | ZNF28 | 24 | 3122 | 3146 | 0.8 | 0.16 | |
| 26. | | DPPA2 | 26 | 3120 | 3146 | 0.8 | 0.021 | |
| 27. | | CHST6 | 21 | 3125 | 3146 | 0.7 | 0.064 | |
| 28. | | FEZ2 | 8 | 3138 | 3146 | 0.3 | 0.29 | |
| 29. | | KRAS | 209 | 2937 | 3146 | 6.6 | 0.0012 | Good |
| 30. | | CDKN2A | 116 | 3030 | 3146 | 3.7 | 0.012 | |
| 31. | | DNMT3A | 97 | 3049 | 3146 | 3.08328 | 1.20E−08 | |
| 32. | | FLT3 | 93 | 3053 | 3146 | 2.95613 | 6.96E−06 | |
| 33. | | NFE2L2 | 75 | 3071 | 3146 | 2.38398 | 0.26 | |
| 34. | | NPM1 | 61 | 3085 | 3146 | 1.93897 | 1.11E−16 | |
| 35. | | MIR142 | 6 | 3140 | 3146 | 0.19072 | 0.48 | |
| 36. | | FOXL2 | 1 | 3145 | 3146 | 0.03179 | 0.26 | |
| 37. | | H3F3A | 6 | 3140 | 3146 | 0.19072 | 0.69 | |
| 38. | | H3F3B | 8 | 3138 | 3146 | 0.25429 | 0.19 | |
| 39. | | KMT2D | ND | | | | | |
| 40. | | RNF43 | 39 | 3107 | 3146 | 1.23967 | 0.61 | |
| 41. | | TERT | 21 | 3125 | 3146 | 0.66751 | 0.0031 | |
| 42. | | ERBB2 | 59 | 3087 | 3146 | 1.8754 | 0.59 | |
| 43. | | PLCG1 | 43 | 3103 | 3146 | 1.36682 | 0.48 | |

(Section H, with rows continued)

| | | | | | | BCM Percent in | P value Somatic |
|---|---|---|---|---|---|---|---|
| | | | Poor | | | poor prognosis | non-silent |
| 1. | BCM | Gene | prognosis | Good prognosis | | group | mutations |
| 2. | 3517 samples | TP53 | 1041 | 2408 | 3449 | 30.2 | 0.00E+00 |
| 3. | | PCDH15 | 177 | 3272 | 3449 | 5.1 | 0.00061 |
| 4. | | DMD | 159 | 3290 | 3449 | 4.6 | 3.61E−05 |
| 5. | | NF1 | 155 | 3294 | 3449 | 4.5 | 0.004 |
| 6. | | NOTCH1 | 89 | 3360 | 3449 | 2.6 | 0.79 |
| 7. | | EGFR | 82 | 3367 | 3449 | 2.4 | 0.0043 |
| 8. | | MALAT1 | 37 | 3412 | 3449 | 1.1 | 0.0027 |
| 9. | | RB1 | 72 | 3377 | 3449 | 2.1 | 0.019 |
| 10. | | LPHN3 | 92 | 3357 | 3449 | 2.7 | 0.015 |
| 11. | | KDM6A | 43 | 3406 | 3449 | 1.2 | 3.84E−05 |
| 12. | | TLR4 | 71 | 3378 | 3449 | 2.1 | 0.0091 |
| 13. | | KEAP1 | 40 | 3409 | 3449 | 1.2 | 0.037 |
| 14. | | SMAD4 | 124 | 3325 | 3449 | 3.6 | 4.36E−12 |
| 15. | | PRX | 47 | 3402 | 3449 | 1.4 | 0.13 |
| 16. | | EPHA7 | 78 | 3371 | 3449 | 2.3 | 7.45E−09 |
| 17. | | IDH1 | 257 | 3192 | 3449 | 7.5 | 0.38 |
| 18. | | KIAA1244 | 74 | 3375 | 3449 | 2.1 | 0.00036 |
| 19. | | STK11 | 16 | 3433 | 3449 | 0.5 | 0.013 |
| 20. | | PTPN11 | 43 | 3406 | 3449 | 1.2 | 0.0023 |
| 21. | | ELF3 | 31 | 3418 | 3449 | 0.9 | 0.064 |
| 22. | | VEZF1 | 18 | 3431 | 3449 | 0.5 | 0.41 |
| 23. | | DAB2IP | 34 | 3415 | 3449 | 1.0 | 0.063 |
| 24. | | GLUD2 | 40 | 3409 | 3449 | 1.2 | 0.074 |
| 25. | | ZNF28 | 30 | 3419 | 3449 | 0.9 | 5.45E−05 |
| 26. | | DPPA2 | 35 | 3414 | 3449 | 1.0 | 0.21 |
| 27. | | CHST6 | 22 | 3427 | 3449 | 0.6 | 0.038 |
| 28. | | FEZ2 | 29 | 3420 | 3449 | 0.8 | 0.92 |
| 29. | | KRAS | 317 | 3132 | 3449 | 9.2 | 5.12E−11 |
| 30. | | CDKN2A | 134 | 3315 | 3449 | 3.9 | 0.0042 |
| 31. | | DNMT3A | 43 | 3406 | 3449 | 1.24674 | 0.31 |
| 32. | | FLT3 | 58 | 3391 | 3449 | 1.68165 | 0.18 |
| 33. | | NFE2L2 | 42 | 3407 | 3449 | 1.21774 | 0.012 |
| 34. | | NPM1 | 9 | 3440 | 3449 | 0.26095 | 0.99 |
| 35. | | MIR142 | NO DATA | | | | |
| 36. | | FOXL2 | 11 | 3438 | 3449 | 0.31893 | 0.72 |
| 37. | | H3F3A | 6 | 3443 | 3449 | 0.17396 | 0.024 |
| 38. | | H3F3B | 2 | 3447 | 3449 | 0.05799 | 0.51 |
| 39. | | KMT2D | NO DATA | | | | |
| 40. | | RNF43 | 90 | 3359 | 3449 | 2.60945 | 0.065 |
| 41. | | TERT | 24 | 3425 | 3449 | 0.69585 | 0.18 |
| 42. | | ERBB2 | 55 | 3394 | 3449 | 1.59467 | 2.48E-06 |
| 43. | | PLCG1 | 57 | 3392 | 3449 | 1.65265 | 0.002 |

(Section I, with rows continued)

| | | | | | | BCGSC Percent in | P value Somatic |
|---|---|---|---|---|---|---|---|
| | | | Poor | | | poor prognosis | non-silent |
| 1. | BCGSC | Gene | prognosis | Good prognosis | | group | mutations |
| 2. | 1947 samples | TP53 | 630 | 1304 | 1934 | 32.6 | 0.00E+00 |
| 3. | | PCDH15 | 98 | 1836 | 1934 | 5.1 | 0.00047 |
| 4. | | DMD | 92 | 1842 | 1934 | 4.8 | 0.0018 |
| 5. | | NF1 | 59 | 1875 | 1934 | 3.1 | 0.51 |
| 6. | | NOTCH1 | 81 | 1853 | 1934 | 4.2 | 0.00062 |
| 7. | | EGFR | 31 | 1903 | 1934 | 1.6 | 0.054 |
| 8. | | MALAT1 | 48 | 1886 | 1934 | 2.5 | 0.014 |
| 9. | | RB1 | 59 | 1875 | 1934 | 3.1 | 0.46 |
| 10. | | LPHN3 | 40 | 1894 | 1934 | 2.1 | 0.35 |
| 11. | | KDM6A | 83 | 1851 | 1934 | 4.3 | 0.069 |
| 12. | | TLR4 | 27 | 1907 | 1934 | 1.4 | 0.61 |
| 13. | | KEAP1 | 33 | 1901 | 1934 | 1.7 | 0.085 |
| 14. | | SMAD4 | 49 | 1885 | 1934 | 2.5 | 2.17E−05 |
| 15. | | PRX | 26 | 1908 | 1934 | 1.3 | 0.42 |
| 16. | | EPHA7 | 41 | 1893 | 1934 | 2.1 | 0.019 |
| 17. | | IDH1 | 19 | 1915 | 1934 | 1.0 | 0.0087 |
| 18. | | KIAA1244 | 22 | 1912 | 1934 | 1.1 | 0.06 |
| 19. | | STK11 | 5 | 1929 | 1934 | 0.3 | 0.095 |
| 20. | | PTPN11 | 36 | 1898 | 1934 | 1.9 | 0.65 |
| 21. | | ELF3 | 53 | 1881 | 1934 | 2.7 | 0.038 |
| 22. | | VEZF1 | 14 | 1920 | 1934 | 0.7 | 0.55 |
| 23. | | DAB2IP | 15 | 1919 | 1934 | 0.8 | 0.3 |
| 24. | | GLUD2 | 18 | 1916 | 1934 | 0.9 | 0.67 |
| 25. | | ZNF28 | 34 | 1900 | 1934 | 1.8 | 0.0063 |
| 26. | | DPPA2 | 17 | 1917 | 1934 | 0.9 | 0.024 |
| 27. | | CHST6 | 11 | 1923 | 1934 | 0.6 | 0.2 |
| 28. | | FEZ2 | 3 | 1931 | 1934 | 0.2 | 0.017 |
| 29. | | KRAS | 138 | 1796 | 1934 | 7.1 | 1.05E−14 |
| 30. | | CDKN2A | 96 | 1838 | 1934 | 5.0 | 0.048 |
| 31. | | DNMT3A | 45 | 3076 | 3121 | 1.44185 | 0.36 |
| 32. | | FLT3 | 43 | 3078 | 3121 | 1.37776 | 0.041 |
| 33. | | NFE2L2 | 92 | 3029 | 3121 | 2.94777 | 0.00024 |
| 34. | | NPM1 | 12 | 3109 | 3121 | 0.38449 | 0.13 |
| 35. | | MIR142 | NO DATA | | | | |
| 36. | | FOXL2 | 5 | 3116 | 3121 | 0.16021 | 0.24 |
| 37. | | H3F3A | 4 | 3117 | 3121 | 0.12816 | 0.012 |
| 38. | | H3F3B | 14 | 3107 | 3121 | 0.44857 | 0.72 |
| 39. | | KMT2D | NO DATA | | | | |
| 40. | | RNF43 | 52 | 3069 | 3121 | 1.66613 | 0.87 |
| 41. | | TERT | 20 | 3101 | 3121 | 0.64082 | 0.15 |
| 42. | | ERBB2 | 96 | 3025 | 3121 | 3.07594 | 0.02 |
| 43. | | PLCG1 | 54 | 3067 | 3121 | 1.73021 | 0.049 |

(Section J, with rows continued)

| | | | | | | Xena-3 | |
|---|---|---|---|---|---|---|---|
| | | | | | | Pancancer29 | |
| | Xena-3 (11.11.2015 | | Poor | | | Percent in poor | P value Somatic |
| 1. | version) | Gene | prognosis | Good prognosis | | prognosis group | non-silent mutations |
| 2. | 8542 samples | TP53 | 2992 | 5280 | 8272 | 36.2 | 0.00E+00 |
| 3. | | PCDH15 | 510 | 7762 | 8272 | 6.2 | 0.01 |
| 4. | | DMD | 517 | 7755 | 8272 | 6.3 | 0.32 |
| 5. | | NF1 | 400 | 7872 | 8272 | 4.8 | 0.012 |
| 6. | | NOTCHI | 285 | 7987 | 8272 | 3.4 | 0.054 |
| 7. | | EGFR | 294 | 7978 | 8272 | 3.6 | 7.45E−13 |
| 8. | | MALATI | 286 | 7986 | 8272 | 3.5 | 0.0065 |
| 9. | | RBI | 309 | 7963 | 8272 | 3.7 | 0.031 |
| 10. | | LPHN3 | 251 | 8021 | 8272 | 3.0 | 0.041 |
| 11. | | KDM6A | 233 | 8039 | 8272 | 2.8 | 0.00079 |
| 12. | | TLR4 | 205 | 8067 | 8272 | 2.5 | 0.1 |
| 13. | | KEAPI | 199 | 8073 | 8272 | 2.4 | 0.0051 |
| 14. | | SMAD4 | 198 | 8074 | 8272 | 2.4 | 2.68E−06 |
| 15. | | PRX | 133 | 8139 | 8272 | 1.6 | 0.52 |
| 16. | | EPHA7 | 178 | 8094 | 8272 | 2.2 | 0.0016 |
| 17. | | IDHI | 498 | 7774 | 8272 | 6.0 | 0.00089 |
| 18. | | KIAA1244 | 163 | 8109 | 8272 | 2.0 | 0.028 |
| 19. | | STK11 | 115 | 8157 | 8272 | 1.4 | 0.0002 |
| 20. | | PTPN11 | 82 | 8190 | 8272 | 1.0 | 0.00015 |
| 21. | | ELF3 | 107 | 8165 | 8272 | 1.3 | 0.099 |
| 22. | | VEZF1 | 70 | 8202 | 8272 | 0.8 | 0.65 |
| 23. | | DAB2IP | 85 | 8187 | 8272 | 1.0 | 0.34 |
| 24. | | GLUD2 | 96 | 8176 | 8272 | 1.2 | 0.09 |
| 25. | | ZNF28 | 86 | 8186 | 8272 | 1.0 | 0.4 |
| 26. | | DPPA2 | 76 | 8196 | 8272 | 0.9 | 0.13 |
| 27. | | CHST6 | 56 | 8216 | 8272 | 0.7 | 0.14 |
| 28. | | FEZ2 | 30 | 8242 | 8272 | 0.4 | 0.11 |
| 29. | | KRAS | 586 | 7686 | 8272 | 7.1 | 3.40E−06 |
| 30. | | CDKN2A | 318 | 7954 | 8272 | 3.8 | 1.97E−05 |
| 31. | | DNMT3A | 202 | 8070 | 8272 | 2.4 | 0.0016 |
| 32. | | FLT3 | 189 | 8083 | 8272 | 2.3 | 3.47E−06 |
| 33. | | NFE2L2 | 172 | 8100 | 8272 | 2.1 | 0.0023 |
| 34. | | NPM1 | 78 | 8194 | 8272 | 0.9 | 2.71E−10 |
| 35. | | MIR142 | 6 | 8266 | 8272 | 0.1 | 0.036 |
| 36. | | FOXL2 | 24 | 8248 | 8272 | 0.3 | 0.017 |
| 37. | | H3F3A | 20 | 8252 | 8272 | 0.2 | 0.004 |
| 38. | | H3F3B | 27 | 8245 | 8272 | 0.3 | 0.016 |
| 39. | | KMT2D | 418 | 3694 | 4112 | 10.2 | 0.0013 |
| 40. | | RNF43 | 73 | 8199 | 8272 | 0.9 | 0.047 |
| 41. | | TERT | 71 | 8201 | 8272 | 0.9 | 0.054 |
| 42. | | ERBB2 | 189 | 8083 | 8272 | 2.3 | 0.058 |
| 43. | | PLCG1 | 127 | 8145 | 8272 | 1.5 | 0.053 |
| | | 33 of 42 | SCARs regulated gene | | | | |
| | | 78.57142857 | | | | | |

TABLE 16
SNMsPvalues.

| | SNMs p value | | | | | | | | | | | | |
|---|---|---|---|---|---|---|---|---|---|---|---|---|---|
| | | | | UCSC | | | | | | | | | |
| | | | | automated | | | | | | | | | |
| | | | Broad- | vcf | | Intenational Cancer | | Baylor | | | | | |
| | Xena-1 | | MIT | UCSC | Xena-2 | genome Consortium | | College of | British Columbia Genome Science Center | | | | |
| | SNMs | Pancan19 | Broad- | automated | SNMs | ICGC | | Medicien | | SNMs | | | |
| | Xena-1.0 | Pancan19 | MIT | vcf | Xena-2.0 | Pancancer | Pancan12 | BCM | BCGSC | Xena-3.0 | | | |
| | Number of samples (K-M survival curves) | | | | | | | | | | | | |
| Gene | 7,075 | 4,232 | 4,228 | 2,907 | 6,945 | 2,538 | 3,146 | 3,449 | 1,934 | 8,272 | Gene | p = <0.05 | p = <0.1 |
| TP53 | 0.00E+00 | 1.42E−11 | 2.56E−06 | 7.13E−12 | 1.37E−06 | 0.00E+00 | 0.0002 | 0.00E+00 | 0.00E+00 | 0.00E+00 | TP53 | 10 | 10 |
| PCDH15 | 2.77E−05 | 0.0133 | 0.65 | 0.22 | 0.42 | 0.31 | 0.99 | 0.00061 | 0.00047 | 0.01 | PCDH15 | 5 | 5 |
| DMD | 0.031 | 0.88 | 0.83 | 0.046 | 0.6 | 0.13 | 0.44 | 3.61E−05 | 0.0018 | 0.32 | DMD | 4 | 4 |
| NF1 | 3.93E−06 | 0.015 | 0.00069 | 0.0031 | 0.016 | 0.36 | 0.27 | 0.004 | 0.51 | 0.012 | NF1 | 7 | 7 |
| NOTCH1 | 0.016 | 0.013 | 0.57 | 0.77 | 0.99 | 0.51 | 0.067 | 0.79 | 0.00062 | 0.054 | NOTCH1 | 4 | 5 |
| EGFR | 0.00E+00 | 0.00E+00 | 0.00E+00 | 0.0078 | 0.00E+00 | 0.16 | 0.00E+00 | 0.0043 | 0.054 | 7.45E−13 | EGFR | 8 | 9 |
| MALAT1 | 0.00043 | 0.011 | 0.87 | 0.27 | 0.81 | 0.63 | 0.002 | 0.0027 | 0.014 | 0.0065 | MALAT1 | 6 | 6 |
| RB1 | 0.00059 | 0.85 | 0.037 | 0.58 | 0.71 | 0.13 | 0.73 | 0.019 | 0.46 | 0.031 | RB1 | 4 | 4 |
| LPHN3 | 0.0094 | 0.65 | 0.057 | 0.024 | 0.3 | 0.28 | 0.48 | 0.015 | 0.35 | 0.041 | LPHN3 | 4 | 5 |
| KDM6A | 9.93E−05 | 0.58 | 0.55 | 0.091 | 0.28 | 0.11 | 0.44 | 3.84E−05 | 0.069 | 0.00079 | KDM6A | 3 | 4 |
| TLR4 | 0.031 | 0.22 | 0.32 | 0.11 | 0.97 | 0.029 | 0.88 | 0.0091 | 0.61 | 0.1 | TLR4 | 3 | 4 |
| KEAP1 | 0.00011 | 0.12 | 0.66 | 0.31 | 0.93 | 0.96 | 0.23 | 0.037 | 0.085 | 0.0051 | KEAP1 | 3 | 4 |
| SMAD4 | 2.58E−08 | 0.034 | 0.19 | 0.00012 | 0.00069 | 2.22E−15 | 0.92 | 4.36E−12 | 2.17E−05 | 2.68E−06 | SMAD4 | 8 | 8 |
| PRX | 0.01 | 0.21 | 0.65 | 0.76 | 0.67 | 0.047 | 0.87 | 0.13 | 0.42 | 0.52 | PRX | 2 | 2 |
| EPHA7 | 2.53E−05 | 0.38 | 0.71 | 0.015 | 0.48 | 0.92 | 0.74 | 7.45E−09 | 0.019 | 0.0016 | EPHA7 | 5 | 5 |
| IDH1 | 0.0015 | 0.12 | 0.48 | 5.28E−05 | 5.45E−05 | 0.11 | 0.91 | 0.38 | 0.0087 | 0.00089 | IDH1 | 5 | 5 |
| KIAA1244 | 0.0064 | 0.99 | 0.65 | 0.093 | 1 | 0.05 | 0.85 | 0.00036 | 0.06 | 0.028 | KIAA1244 | 4 | 5 |
| STK11 | 0.00011 | 0.013 | 0.23 | 0.81 | 0.0028 | 1.15E−05 | 0.011 | 0.013 | 0.095 | 0.0002 | STK11 | 7 | 8 |
| PTPN11 | 0.00023 | 0.11 | 0.025 | 0.46 | 0.43 | 0.35 | 0.36 | 0.0023 | 0.65 | 0.00015 | PTPN11 | 4 | 4 |
| ELF3 | 0.02 | 0.81 | 0.77 | 0.41 | 0.52 | 0.26 | 0.95 | 0.064 | 0.038 | 0.099 | ELF3 | 2 | 4 |
| VEZF1 | 0.019 | 0.12 | 0.84 | 0.68 | 0.33 | 0.95 | 0.23 | 0.41 | 0.55 | 0.65 | VEZF1 | 1 | 1 |
| DAB2IP | 4.21E−05 | 0.0084 | 0.19 | 0.5 | 0.89 | 0.72 | 0.26 | 0.063 | 0.3 | 0.34 | DAB2IP | 2 | 3 |
| GLUD2 | 0.024 | 0.39 | 0.27 | 0.43 | 0.3 | 0.97 | 0.7 | 0.074 | 0.67 | 0.09 | GLUD2 | 1 | 3 |
| ZNF28 | 0.012 | 0.24 | 4.33E−06 | 0.49 | 0.59 | 0.17 | 0.16 | 5.45E−05 | 0.0063 | 0.4 | ZNF28 | 4 | 4 |
| DPPA2 | 0.032 | 0.054 | 0.18 | 0.7 | 0.14 | 0.36 | 0.021 | 0.21 | 0.024 | 0.13 | DPPA2 | 3 | 4 |
| CHST6 | 0.039 | 0.22 | 0.19 | 0.67 | 0.22 | 0.31 | 0.064 | 0.038 | 0.2 | 0.14 | CHST6 | 2 | 3 |
| FEZ2 | 0.014 | 0.26 | 0.99 | 0.53 | 0.91 | 0.46 | 0.29 | 0.92 | 0.017 | 0.11 | FEZ2 | 2 | 2 |
| KRAS | NS | 1 | 0.023 | 1.11E−16 | 0.001 | 0.00E+00 | 0.0012 | 5.12E−11 | 1.05E−14 | 3.40E−06 | KRAS | 6 | 8 |
| CDKN2A | NS | 0.015 | 2.32E−05 | 0.074 | 6.84E−11 | 2.33E−12 | 0.012 | 0.0042 | 0.048 | 1.97E−05 | CDKN2A | 8 | 9 |
| DNMT3A | NS | 3.42E−07 | 0.00017 | 0.11 | NS | 0.87 | 1.20E−08 | 0.31 | 0.36 | 0.0016 | DNMT3A | 4 | 4 |
| FLT3 | NS | 0.001 | 0.15 | 0.63 | NS | 0.71 | 6.96E−06 | 0.18 | 0.041 | 3.47E−06 | FLT3 | 4 | 4 |
| NFE2L2 | NS | 0.15 | 1.60E−09 | 0.97 | NS | 0.29 | 0.26 | 0.012 | 0.00024 | 0.0023 | NFE2L2 | 4 | 4 |
| NPM1 | NS | 6.48E−11 | 0.2 | 0.22 | NS | 0.096 | 1.11E−16 | 0.99 | 0.13 | 2.71E−10 | NPM1 | 3 | 4 |
| MIR142 | NS | 0.3 | ND | 0.25 | NS | 0.18 | 0.48 | ND | ND | 0.036 | MIR142 | 1 | 1 |
| FOXL2 | ND | 0.0058 | 0.034 | 0.055 | ND | 0.81 | 0.26 | 0.72 | 0.24 | 0.017 | FOXL2 | 3 | 4 |
| H3F3A | ND | 0.97 | 0.018 | 0.31 | ND | 0.29 | 0.69 | 0.024 | 0.012 | 0.004 | H3F3A | 4 | 4 |
| H3F3B | ND | 0.1 | 0.015 | 0.43 | ND | 0.46 | 0.19 | 0.51 | 0.72 | 0.016 | H3F3B | 2 | 3 |
| KMT2D | ND | ND | 0.029 | 0.25 | ND | 0.17 | ND | ND | ND | 0.0013 | KMT2D | 2 | 2 |
| RNF43 | ND | 0.7 | 0.6 | 0.11 | ND | 0.00072 | 0.61 | 0.065 | 0.87 | 0.047 | RNF43 | 3 | 3 |
| TERT | ND | 0.0021 | 0.012 | 0.19 | ND | 0.8 | 0.0031 | 0.18 | 0.15 | 0.054 | TERT | 3 | 4 |
| ERBB2 | ND | 0.57 | 0.12 | 0.0059 | ND | 0.19 | 0.59 | 2.48E−06 | 0.02 | 0.058 | ERBB2 | 2 | 3 |
| PLCG1 | ND | 0.67 | 0.0057 | 0.12 | ND | 0.78 | 0.48 | 0.002 | 0.049 | 0.053 | PLCG1 | 5 | 4 |
| | | | | UCSC | | | | | | | | | |
| | SNMs | | | automated | SNMs | ICGC | | | | SNMs | | | |
| Gene | Xena-1.0 | Pancan19 | Broad-MIT | vcf | Xena-2.0 | Pancancer | Pancancer12 | BCM | BCGSC | Xena-3.0 | Gene | | |
| | Number of samples in dataset | | | | | | | | | | Number of | | |
| | 7,509 | 4,429 | 4,333 | 2,970 | 7,173 | 3,453 | 3,276 | 3,517 | 1,947 | 8,542 | samples in dataset | | |
| NS, not significant; ND, | | | | | | | | | | | | | |
| no data | | | | | | | | | | | | | |
| Significant associations with survival | | | | | | | | | | | | | |
| VEZF1 | ZNF161 | | | | | | | | | | | | |
| GLUD2 | | | | | | | | | | | | | |
| Gene expression TCGA breast | | | | | | | | | | | | | |
| cancer | | | | | | | | | | | | | |
| Gene expression TCGA Glioblastoma | | | | | | | | | | | | | |
| PANCANCER 12 K | | | | | | | | | | | | | |
| Gene level copy number changes | | | | | | | | | | | | | |
| Gene | | | | | | | | | | | | | |
| expression | | | | | | | | | | | | | |
| | SNMs | TCGA | Broad- | UCSC | SNMs | Intenational Cancer | | Baylor | British Columbia | SNMs | | | |
| | Xena-1 | Panncan19 | MIT | automated | Xena-2 | genome Consortium | | College of | Genome Science | Xena-3.0 | | | |
| | | | | vcf | | | | Medicien | Center | | | | |
| | TCGA | Panncan19 | Broad- | UCSC | TCGA | ICGC | Pancan12 | BCM | BCGSC | | | | |
| | Xena-1.0 | | MIT | automated | Xena-2.0 | Pancancer | | | | | | | |
| | | | | vcf | | | | | | | | | |
| | | TCGA | TCGA | TCGA | TCGA Pan-cacer | | | TCGA | TCGA Pan-cacer | TCGA Pan-cacer | | | |
| | | Pan-cacer | Pan- | Pan-cacer | | | | Pan-cacer | | | | | |
| | | | cacer | | | | | | | | | | |

| | |
|---|---|
| Public 10.30.15 | Public 11.11.15 |

TABLE 17
PercentSNMs.

| | Percent of patients with gene-level somatic non-silent mutations (SNMs) | | | | | | | | | | | |
|---|---|---|---|---|---|---|---|---|---|---|---|---|
| | 19 cohorts | Xena-1 | Xena-2 | | | | Pancancer | | | Xena-3 | | |
| | Pancancer19 | Pancancer29 | Pancancer29 | Broad | | ICGC | 12 cohorts | BCM | BCGSC | Pancancer29 | | |
| | Percent in | Percent in | Percent in | Percent in | | Pancancer | Percent in | Percent | Percent | Percent in | | |
| | poor | poor | poor | poor | | in poor | poor | in poor | in poor | poor | | |
| | prognosis | prognosis | prognosis | prognosis | Pancancer | prognosis | prognosis | prognosis | prognosis | prognosis | | Average |
| Gene | group | group | group | group | UCSC | group | group | group | group | group | Gene | (n = 10) |
| TP53 | 35.8459 | 37.1731 | 21.7279 | 11.5658 | 24.2174 | 37.7069 | 41.8309 | 30.1827 | 32.575 | 36.1702 | TP53 | 30.9 |
| PCDH15 | 6.3327 | 7.27915 | 2.98056 | 1.46641 | 7.08634 | 3.30969 | 5.1494 | 5.13192 | 5.06722 | 6.16538 | PCDH15 | 5.0 |
| DMD | 6.00189 | 6.57244 | 3.94528 | 2.15232 | 6.67355 | 2.32467 | 6.42085 | 4.61003 | 4.75698 | 6.25 | DMD | 5.0 |
| NF1 | 5.05671 | 5.5689 | 2.67819 | 2.03406 | 4.16237 | 2.20646 | 4.92689 | 4.49406 | 3.05067 | 4.83559 | NF1 | 3.9 |
| NOTCH1 | 3.40265 | 4.21201 | 1.64147 | 0.75686 | 4.33437 | 2.04886 | 3.33757 | 2.58046 | 4.18821 | 3.44536 | NOTCH1 | 3.0 |
| EGFR | 4.37146 | 4.14134 | 2.17423 | 2.12867 | 3.40557 | 0.51221 | 4.86332 | 2.3775 | 1.6029 | 3.55416 | EGFR | 2.9 |
| MALAT1 | 3.59168 | 3.91519 | 0.99352 | 0.6386 | 4.26557 | 2.56107 | 2.76542 | 1.07277 | 2.4819 | 3.45745 | MALAT1 | 2.6 |
| RB1 | 3.11909 | 3.90106 | 1.78546 | 1.06939 | 1.78879 | 1.73365 | 3.62365 | 2.08756 | 3.05067 | 3.73549 | RB1 | 2.6 |
| LPHN3 | 2.95369 | 3.42049 | 1.46868 | 0.61495 | 2.75198 | 2.22036 | 2.95613 | 2.66744 | 2.06825 | 3.03433 | LPHN3 | 2.4 |
| KDM6A | 2.12665 | 3.15194 | 1.49748 | 0.99338 | 2.13278 | 1.81245 | 2.35219 | 1.24674 | 4.29162 | 2.81673 | KDM6A | 2.2 |
| TLR4 | 2.4811 | 2.71378 | 1.05112 | 0.37843 | 2.61438 | 0.74862 | 2.22505 | 2.05857 | 1.39607 | 2.47824 | TLR4 | 1.8 |
| KEAP1 | 2.12665 | 2.61484 | 0.79194 | 0.6386 | 1.68559 | 1.06383 | 2.54291 | 1.15976 | 1.70631 | 2.40571 | KEAP1 | 1.7 |
| SMAD4 | 1.74858 | 2.50177 | 1.91505 | 0.18921 | 2.33918 | 6.30418 | 1.77665 | 3.59524 | 2.53361 | 2.39362 | SMAD4 | 2.5 |
| PRX | 1.70132 | 2.17668 | 0.92153 | 0.26017 | 2.37358 | 1.02443 | 1.27146 | 1.36271 | 1.34436 | 1.60783 | PRX | 1.4 |
| EPHA7 | 2.12665 | 2.23322 | 0.90713 | 0.40208 | 2.92398 | 1.89125 | 1.90718 | 2.26153 | 2.11996 | 2.15184 | EPHA7 | 1.9 |
| IDH1 | 4.67864 | 6.86926 | 6.13391 | 0.49669 | 14.5855 | 0.78802 | 1.65289 | 7.45144 | 0.98242 | 6.02031 | IDH1 | 5.0 |
| KIAA1244 | 1.63043 | 2.10601 | 1.18071 | 0.44939 | 3.02718 | 1.13843 | 1.33503 | 2.14555 | 1.13754 | 1.9705 | KIAA1244 | 1.6 |
| STK11 | 0.82703 | 1.61131 | 0.53276 | 0.49669 | 0.92879 | 0.23641 | 0.89002 | 0.4639 | 0.25853 | 1.39023 | STK11 | 0.8 |
| PTPN11 | 1.15784 | 0.89046 | 0.70554 | 0.26017 | 2.06398 | 0.63042 | 1.04895 | 1.24674 | 1.86143 | 0.9913 | PTPN11 | 1.1 |
| ELF3 | 0.77977 | 1.35689 | 0.57595 | 0.09461 | 0.85999 | 0.55162 | 0.6993 | 0.89881 | 2.74043 | 1.29352 | ELF3 | 1.0 |
| VEZF1 | 0.66163 | 1.08834 | 0.50396 | 0.21287 | 0.344 | 0.1182 | 0.60394 | 0.52189 | 0.72389 | 0.84623 | VEZF1 | 0.6 |
| DAB2IP | 1.06333 | 1.35689 | 0.63355 | 0.14191 | 1.85759 | 0.31521 | 0.82645 | 0.98579 | 0.77559 | 1.02756 | DAB2IP | 0.9 |
| GLUD2 | 1.06333 | 1.28622 | 0.79194 | 0.47304 | 1.47919 | 0.35461 | 1.14431 | 1.15976 | 0.93071 | 1.16054 | GLUD2 | 1.0 |
| ZNF28 | 0.92155 | 1.15901 | 0.46076 | 0.24225 | 1.06639 | 0.35461 | 0.76287 | 0.86982 | 1.75801 | 1.03965 | ZNF28 | 0.9 |
| DPPA2 | 0.99244 | 1.04594 | 0.40317 | 0.26017 | 1.16959 | 0.1576 | 0.82645 | 1.01479 | 0.87901 | 0.91876 | DPPA2 | 0.8 |
| CHST6 | 0.638 | 0.73498 | 0.48956 | 0.21287 | 0.6192 | 0.47281 | 0.66751 | 0.63787 | 0.56877 | 0.67698 | CHST6 | 0.6 |
| FEZ2 | 0.21267 | 0.42403 | 0.28798 | 0.11826 | 0.2752 | 0.19701 | 0.25429 | 0.84082 | 0.15512 | 0.36267 | FEZ2 | 0.3 |
| KRAS | 5.88374 | | 5.55796 | 1.30085 | 5.98555 | 23.2072 | 6.64336 | 9.19107 | 7.13547 | 7.08414 | KRAS | 8.0 |
| CDKN2A | 3.80435 | | 1.45428 | 1.13529 | 1.65119 | 5.51615 | 3.68722 | 3.88518 | 4.96381 | 3.84429 | CDKN2A | 3.3 |
| DNMT3A | 2.69376 | | | 1.78725 | 2.09838 | 0.66982 | 3.08328 | 0.31 | 1.44185 | 0.0016 | DNMT3A | 1.5 |
| FLT3 | 2.93006 | | | 1.80443 | 2.37358 | 0.70922 | 2.95613 | 0.18 | 1.37776 | 3.5E−06 | FLT3 | 1.5 |
| NFE2L2 | 2.0794 | | | 2.57776 | 1.89198 | 1.65485 | 2.38398 | 0.012 | 2.94777 | 0.0023 | NFE2L2 | 1.7 |
| NPM1 | 1.53592 | | | 0.29215 | 0.6192 | 0.27581 | 1.93897 | 0.99 | 0.38449 | 2.7E−10 | NPM1 | 0.8 |
| M1R142 | 0.14178 | | | | 0.1032 | 0.27581 | 0.19072 | | | 0.036 | M1R142 | 0.1 |
| FOXL2 | 0.16541 | | | 0.22341 | 0.172 | 0.1182 | 0.03179 | 0.31893 | 0.16021 | 0.29014 | FOXL2 | 0.2 |
| H3F3A | 0.23629 | | | 0.20622 | 0.3096 | 0.1182 | 0.19072 | 0.17396 | 0.12816 | 0.24178 | H3F3A | 0.2 |
| H3F3B | 0.25992 | | | 0.41244 | 0.2408 | 0.19701 | 0.25429 | 0.05799 | 0.44857 | 0.3264 | H3F3B | 0.3 |
| KMT2D | | | | 7.26929 | 7.36154 | 4.25532 | | | | 10.1654 | KMT2D | 7.3 |
| RNF43 | 1.25236 | | | 1.65062 | 1.75439 | 1.77305 | 1.23967 | 2.60945 | 1.66613 | 0.8825 | RNF43 | 1.6 |
| TERT | 0.87429 | | | 0.96236 | 1.37599 | 0.59102 | 0.66751 | 0.69585 | 0.64082 | 0.85832 | TERT | 0.8 |
| ERBB2 | 1.70132 | | | 2.09658 | 2.95838 | 0.82742 | 1.8754 | 1.59467 | 3.07594 | 2.28482 | ERBB2 | 2.1 |
| PLCG1 | 1.46503 | | | 1.37481 | 2.23598 | 0.66982 | 1.36682 | 1.65265 | 1.73021 | 1.5353 | PLCG1 | 1.5 |
| Gene | 19 cohorts | Xena-1 | Xena-2 | Broad | Pancancer | ICGC | Pancancer | BCM | BCGSC | Xena-3 | Gene | |
| | Pancancer19 | Pancancer29 | Pancancer29 | Percent | UCSC | Pancancer | 12 cohorts | Percent | Percent | Pancancer29 | | |
| | Percent in | Percent in | Percent in | with | | in poor | Percent | in poor | in poor | Percent in | | |
| | poor | poor | poor | mutations | | prognosis | with | prognosis | prognosis | poor | | |
| | prognosis | prognosis | prognosis | | | group | mutations | group | group | prognosis | | |
| | group | group | group | | | | | | | group | | |

Note: Tables 4-9 are “Data Set S1”, Tables 10-14 are “Data Set S2”, and Tables 15-17 are “Data Set S3”.

PARAGRAPH 1: A method for diagnosing cancer or predicting cancer-therapy outcome in a subject, comprising: generating target marker information responsive to one or more inputs indicative of a genomic signature pathway and one or more inputs indicative of a proteomic signature pathway of endogenous human Stem Cell-Associated Retroviruses (SCAR); and generating aberrant object information responsive to comparing detected expression levels and sequence information of a biological sample with target marker information.

In an embodiment, generating aberrant object information includes displaying the aberrant object information on a client device, a user interface, and the like. In an embodiment, generating aberrant object information includes exchanging the aberrant object information with a remote network. Non-limiting examples of aberrant object information include aberrant sequence information, aberrant expression level information, expression level is above a target threshold information, detected positioning of a plurality of bases, sequence aberrant score, and the like.

Further non-limiting examples of aberrant object information includes information indicative of a threshold level derived by comparing reference information derived from samples obtained from biological subjects; information indicative of a comparison of at least one input indicative of an expression levels and at least one input indicative of a sequence of a biological sample with target marker information; and the like.

PARAGRAPH 2: The method of according to PARAGRAPH 1, wherein generating the target marker information includes generating target marker information responsive to one or more inputs indicative of a SCARs pathway.

PARAGRAPH 3: The method of according to PARAGRAPH 1, wherein generating the target marker information includes generating target marker information responsive to one or more inputs indicative of a SCARs pathway target gene.

PARAGRAPH 4: The method of according to PARAGRAPH 1, wherein generating the target marker information includes generating target marker information associated with one or more of ELF3; PCDH15; MALAT1; PTPN11; RB1; CHST6; NF1; VEZF1; TP53; SMAD4; KEAP1; STK11; PRX; ZNF28; IDH1; FEZ2; DPPA2; LPHN3; KIAA1244; EPHA7; EGFR; TLR4; DAB21P; NOTCH1; GLUD2; DMD; KDM6A; KRAS; CDKN2A; DNMT3A; FLT3; NFE2L2; NPM1; MIR142; FOXL2; H3F3A; H3F3B; KMT2D ; RNF43 ; TERT; ERBB2; PLCG1.

PARAGRAPH 5: The method of according to PARAGRAPH 1, wherein generating the target marker information includes generating target marker information associated with one or more of mRNA, RNA, DNA, peptide or protein.

PARAGRAPH 6: The method of according to PARAGRAPH 1, wherein generating the target marker information includes generating target marker information associated with one or more of PLCXD1, HKR1, ZNF283, ADA, AMACR+p63, ANK3, BCL2L1, BIRC5, BMI-1, BUB1, CCNB1, CCND1, CES1, CHAF1A, CRIP1, CRYAB, ESM1, EZH2, FGFR2, FOS, Gbx2, HCFC1, IER3, ITPR1, JUNB, KLF6, KI67, KNTC2, MGC5466, Phc1, RNF2, Suz12, TCF2, TRAP100, USP22, Wnt5A and ZFP36.

PARAGRAPH 7: The method of according to PARAGRAPH 1, wherein generating the aberrant object information includes generating aberrant sequence information when a quality of a sequence associated with the biological sample is distinct as compared with one or more reference sequences.

PARAGRAPH 8: The method of according to PARAGRAPH 1, wherein generating the aberrant object information includes generating aberrant sequence information responsive to one or more inputs indicative of a distinct positioning of a plurality of bases within an entire sequence associated with the biological sample, as compared with one or more reference sequences.

PARAGRAPH 9: The method of according to PARAGRAPH 1, wherein generating the aberrant object information includes generating aberrant sequence information responsive to one or more inputs indicative of a distinct fragment of a sequence associated with the biological sample, as compared with one or more reference sequences.

PARAGRAPH 10: The method of according to PARAGRAPH 1, wherein generating the aberrant object information includes generating aberrant expression level information responsive to one or more inputs indicative of when an expression level exceeds a target threshold.

PARAGRAPH 11: The method of according to PARAGRAPH 1, wherein generating the aberrant object information includes determining expression level aberrant score when a detected expression level is above a target threshold

PARAGRAPH 12: The method of according to PARAGRAPH 1, wherein generating the aberrant object information includes determining a sequence aberrant score when a detected positioning of a plurality of bases associated with the biological sample is distinct compared with a one or more reference sequences.

PARAGRAPH 13: The method of according to PARAGRAPH 1, wherein generating the aberrant object information includes determining a sequence aberrant score responsive to one or more inputs from a next generation sequencing, multicolor quantitative immunofluorescence co-localization analysis, fluorescence in situ hybridization, and quantitative RT-PCR analysis.

PARAGRAPH 14: The method of according to PARAGRAPH 1, wherein generating the aberrant object information includes determining a threshold level by comparing reference information derived from samples obtained from biological subjects with known diagnosis or known clinical outcome after therapies.

PARAGRAPH 15: The method of according to PARAGRAPH 14, further comprising: generating a cancer-therapy efficacy status, cancer therapy progress, a cancer prognosis, a cancer diagnosis responsive to one or more inputs indicative of an aberrant expression and an expression level above a target threshold coefficient of at least two markers.

PARAGRAPH 16: The method of according to PARAGRAPH 1, wherein generating the aberrant object information includes generating aberrant sequence information and marker co-expression level information.

PARAGRAPH 17: The method of according to PARAGRAPH 1, further comprising: generating a cancer-therapy efficacy status responsive to one or more inputs indicative of an aberrant sequence and a threshold marker co-expression level.

PARAGRAPH 18: The method of according to PARAGRAPH 1, further comprising: generating information indicative of the presence or absence of cancer in a biological subject responsive to one or more inputs indicative of an aberrant sequence and a threshold marker co-expression level.

PARAGRAPH 19: A system for diagnosing cancer or predicting cancer-therapy outcome in a subject, comprising: circuitry configured to generate target marker information responsive to one or more inputs indicative of a genomic signature pathway and one or more inputs indicative of a proteomic signature pathway of endogenous human Stem Cell-Associated Retroviruses (SCAR); and circuitry configured to generate aberrant object information responsive to comparing at least one input indicative of an expression levels and at least one input indicative of a sequence of a biological sample with target marker information.

PARAGRAPH 20: The system of according to PARAGRAPH 19, further comprising: circuitry configured to generate information indicative of the presence or absence of cancer in a biological subject responsive to one or more inputs indicative of an aberrant sequence and a threshold marker co-expression level.

PARAGRAPH 21: The system of according to PARAGRAPH 19, further comprising: circuitry configured to generate a cancer-therapy efficacy status, cancer therapy progress, a cancer prognosis, a cancer diagnosis responsive to one or more inputs indicative of an aberrant expression and an expression level above a target threshold coefficient of at least two markers.

PARAGRAPH 22: The system of according to PARAGRAPH 19, further comprising: circuitry configured to generate a cancer-therapy efficacy status responsive to one or more inputs indicative of an aberrant sequence and a threshold marker co-expression level.

PARAGRAPH 23: A system for treating cancer, comprising: circuitry configured to acquire information associated with a Stem Cell-Associated Retroviruses (SCAR) pathway activation in a subject diagnosed with cancer; and circuitry configured to identify single therapeutic agent or combination of therapeutic agents and to generate user-specific treatment protocol responsive to one or more inputs associated with a Stem Cell-Associated Retroviruses (SCAR) pathway activation in a subject diagnosed with cancer.

PARAGRAPH 24: A method for diagnosing cancer or predicting cancer-therapy outcome in a subject, comprising: concurrently screening a biological sample for a presence of an aberrant sequences and an aberrant expression level of one or more target markers associated with a pathway involving genomic and proteomic signatures of endogenous human Stem Cell-Associated Retroviruses (SCAR); scoring a sequence associated with the biological sample as aberrant when the quality of the sequence is distinct compared with a reference sequence; and scoring an expression level associated with the biological sample as being aberrant when a detected expression level is above a target threshold coefficient. In an embodiment, a method for diagnosing cancer or predicting cancer-therapy outcome in a subject, comprising: screening a biological sample for at least one of a presence of an aberrant sequences and an aberrant expression level of one or more target markers associated with a pathway involving genomic and proteomic signatures of endogenous human Stem Cell-Associated Retroviruses (SCAR); scoring a sequence associated with the biological sample as aberrant when the quality of the sequence is distinct compared with a reference sequence; and scoring an expression level associated with the biological sample as being aberrant when a detected expression level is above a target threshold coefficient.

PARAGRAPH 25: The method of according to PARAGRAPH 24, wherein concurrently screening a biological sample for a presence of an aberrant sequences and an aberrant expression level of one or more target markers associated with a pathway involving genomic and proteomic signatures of endogenous SCAR, includes concurrently screening a biological sample for a presence of an aberrant sequences and an aberrant expression level of one or more target markers indicative of a cancer diagnosis or a prognosis for cancer-therapy failure in a biological subject.

PARAGRAPH 26: The method of according to PARAGRAPH 25, further comprising: generating a user-specific cancer therapy protocol responsive to one or more inputs indicative of an aberrant sequence or an aberrant expression level associated with a cancer diagnosis or a prognosis for cancer-therapy failure in a biological subject.

PARAGRAPH 27: The method of according to PARAGRAPH 24, wherein concurrently screening a biological sample for a presence of an aberrant sequences and an aberrant expression level of one or more target markers associated with a pathway involving genomic and proteomic signatures of endogenous SCAR, includes concurrently screening a biological sample for a presence of an aberrant sequences and an aberrant expression level of one or more target markers indicative of a progress of cancer therapy in a biological subject.

PARAGRAPH 28: The method of according to PARAGRAPH 27, further comprising: generating a user-specific cancer therapy protocol responsive to one or more inputs indicative of an aberrant sequence or an aberrant expression level associated with a progress of cancer therapy in a biological subject.

PARAGRAPH 29: The method of according to PARAGRAPH 24, wherein the detection threshold is being determined by comparing to the values in a reference database of samples obtained from subjects with known diagnosis or known clinical outcome after therapies, wherein the presence of an aberrant expression level of at least one but preferably, two or more markers in the test sample and presence of aberrant expression of two or more such markers is indicative of a cancer diagnosis or a prognosis for cancer-therapy failure, or of the progress of cancer therapy in the subject.

PARAGRAPH 30: The method of according to PARAGRAPH 24, where the detection threshold is continuously refined by adding the outcome data of each patient tested to the reference database of samples, and in an automated and/or recursive manner either manually or using computational methods using data stored either locally, in remote server(s), or in the cloud, continuously improving the accuracy of diagnosis, prognosis, or specification of future cancer therapy.

PARAGRAPH 31: The method of according to PARAGRAPH 24, wherein said sample phenotype is selected from the group consisting of cancer, non-cancer, recurrence, non-recurrence, relapse, non-relapse, invasiveness, non-invasiveness, metastatic, non-metastatic, localized, tumor size, tumor grade, Gleason score, survival prognosis, lymph node status, tumor stage, degree of differentiation, age, hormone receptor status, tumor antigen level (including but not limited to PSA level, PSMA level, survivin level, oncofetal protein level, testis antigen level), histologic type, level of, phenotype and genotype of and activation status of immune cells, and disease free survival.

PARAGRAPH 32: The method of according to PARAGRAPH 24, wherein said threshold coefficient has an absolute value 0.5.

PARAGRAPH 33: The method of according to PARAGRAPH 24, wherein said threshold coefficient has an absolute value 0.6.

PARAGRAPH 34: The method of according to PARAGRAPH 24, wherein said threshold coefficient has an absolute value 0.7.

PARAGRAPH 35: The method of according to PARAGRAPH 24, wherein said threshold coefficient has an absolute value 0.8.

PARAGRAPH 36: The method of according to PARAGRAPH 24, wherein said threshold coefficient has an absolute value 0.9.

PARAGRAPH 37: The method of according to PARAGRAPH 24, wherein said threshold coefficient has an absolute value 0.95.

PARAGRAPH 38: The method of according to PARAGRAPH 24, wherein said threshold coefficient has an absolute value 0.99.

PARAGRAPH 39: The method of according to PARAGRAPH 24, wherein said threshold coefficient has an absolute value 0.995.

PARAGRAPH 40: The method of according to PARAGRAPH 24, wherein said threshold coefficient has an absolute value 0.999.

PARAGRAPH 41: A method of determining detection threshold for classifying a sample phenotype, comprising: identifying a subset of markers and scoring marker expression in cells according to the method of according to PARAGRAPH 24; and determining the sample classification accuracy at different detection thresholds using a reference database of samples from subjects with known phenotypes.

PARAGRAPH 42: The method of according to PARAGRAPH 41, comprising determining the sample classification accuracy in an automated and/or recursive manner either manually or using computational methods using data stored either locally, in remote server(s), or in the cloud.

PARAGRAPH 43: The method of according to PARAGRAPH 41, further comprising determining the best performing magnitude of said detection threshold and using said magnitude to assess the reliability of said established detection threshold in classifying a sample phenotype.

PARAGRAPH 44: The method of according to PARAGRAPH 41, further comprising determining the best performing magnitude of said detection threshold and using said magnitude to assess the reliability of said established detection threshold in classifying a sample phenotype in an automated and/or recursive manner either manually or using computational methods using data stored either locally, in remote server(s), or in the cloud.

PARAGRAPH 45: The method of according to PARAGRAPH 41, further comprising using the best performing magnitude of said detection threshold to score an unclassified sample and assign a sample phenotype to said sample.

PARAGRAPH 46: The method of according to PARAGRAPH 41, further comprising using the best performing magnitude of said detection threshold to score an unclassified sample and assign a sample phenotype to said sample either manually or using computational methods using data stored either locally, in remote server(s), or in the cloud.

PARAGRAPH 47: The method of according to PARAGRAPH 41, wherein said subset of markers consists essentially of the genes, genetic loci, and sequences identified in Table 1A, Table 1, Table 2, Table 3, FIGS. 16 and 18A-21C, Data Set S1, Data Set S2, Data Set S3.

PARAGRAPH 48: The method of according to PARAGRAPH 41, wherein said subset of markers consists essentially of 90% of the genes, genetic loci, and sequences identified in Table 1A, Table 1, Table 2, Table 3, FIGS. 16 and 18A-21C, Data Set S1, Data Set S2, Data Set S3.

PARAGRAPH 49: The method of according to PARAGRAPH 41, wherein said subset of markers consists essentially of 80% of the genes, genetic loci, and sequences identified in Table 1A, Table 1, Table 2, Table 3, FIGS. 16 and 18A-21C, Data Set S1, Data Set S2, Data Set S3.

PARAGRAPH 50: The method of according to PARAGRAPH 41, wherein said subset of markers consists essentially of 70% of the genes, genetic loci, and sequences identified in Table 1A, Table 1, Table 2, Table 3, FIGS. 16 and 18A-21C, Data Set S1, Data Set S2, Data Set S3.

PARAGRAPH 51: The method of according to PARAGRAPH 41, wherein said subset of markers consists essentially of 60% of the genes, genetic loci, and sequences identified in Table 1A, Table 1, Table 2, Table 3, FIGS. 16 and 18A-21C, Data Set S1, Data Set S2, Data Set S3.

PARAGRAPH 52: The method of according to PARAGRAPH 41, wherein said subset of markers consists essentially of 50% of the genes, genetic loci, and sequences identified in Table 1A, Table 1, Table 2, Table 3, FIGS. 16 and 18A-21C, Data Set S2, Data Set S3.

PARAGRAPH 53: A method of treating cancer, comprising: detecting a molecular signal(s) of SCAR's pathway activation in a subject diagnosed with cancer; generating a user-specific therapeutic treatment targeted to activated SCAR's loci and/or down-stream SCARs-regulated genetic loci based on detecting the molecular signal(s) of SCAR's pathway activation.

PARAGRAPH 54: The method of according to PARAGRAPH 53, wherein the user-specific therapeutic treatment its based on genome editing, including but not limited to CRISPR/Cas9 complex-mediated genome editing, to silence the defined genomic elements of the activated SCARs pathway.

PARAGRAPH 55: The method of according to PARAGRAPH 53, wherein the user-specific therapeutic treatment is based on genome editing, including but not limited to CRISPR/Cas9 complex-mediated genome editing, to activate the defined genomic elements of the activated SCARs pathway.

PARAGRAPH 56: The method of according to PARAGRAPH 53, wherein the user-specific therapeutic treatment is based on the application of Highly Active Anti-Retroviral Therapy (HAART).

PARAGRAPH 57: The method of according to PARAGRAPH 53, wherein the user-specific therapeutic treatment is based on administration of the antiretroviral drug, Raltegravir (RAL, Isentress, formerly MK-0518).

PARAGRAPH 58: The method of according to PARAGRAPH 53, wherein the user-specific therapeutic treatment is based on application of anti-sense therapy directed against transcriptionally active SCAR's loci and/or defined genomic elements of the activated SCARs pathway.

PARAGRAPH 59: The method of according to PARAGRAPH 53, wherein the user-specific therapeutic treatment is based on the application of targeted immunotherapy, including but not limited to antagonist antibodies or fragments thereof, agonist antibodies or fragments thereof, autologous cells, allogeneic cells, peptides, small molecules, signaling proteins or fragments thereof, or compositions containing two or more of the above and compositions containing in a single molecule or cellular therapy all or part of two or more of the above, directed against the proteins and/or peptides encoded by the activated SCARs sequences.

PARAGRAPH 60: A method of treating cancer where the methods of according to PARAGRAPHs 39-45 are used to enhance tumor infiltrating lymphocytes in tumors of treated subjects, either as a sole function or to augment the activity of anti-cancer modulators of the immune system.

1. A method for diagnosing cancer or predicting cancer-therapy outcome in a subject, comprising:
generating target marker information responsive to one or more inputs indicative of a genomic signature pathway and one or more inputs indicative of a proteomic signature pathway of endogenous human Stem Cell-Associated Retroviruses (SCAR); and
generating aberrant object information responsive to comparing detected expression levels and sequence information of a biological sample with target marker information.

2. The method of claim 1, wherein generating the target marker information includes generating target marker information responsive to one or more inputs indicative of a SCARs pathway.

3. The method of claim 1, wherein generating the target marker information includes generating target marker information responsive to one or more inputs indicative of a SCARs pathway target gene.

4. The method of claim 1, wherein generating the target marker information includes generating target marker information associated with one or more of ELF3; PCDH15; MALAT1; PTPN11; RB1; CHST6; NF1; VEZF1; TP53; SMAD4; KEAP1; STK11; PRX; ZNF28; IDH1; FEZ2; DPPA2; LPHN3; KIAA1244; EPHA7; EGFR; TLR4; DAB2IP; NOTCH1; GLUD2; DMD; KDM6A; KRAS; CDKN2A; DNMT3A; FLT3; NFE2L2; NPM1; MIR142; FOXL2; H3F3A; H3F3B; KMT2D ; RNF43 ; TERT; ERBB2; PLCG1.

5. The method of claim 1, wherein generating the target marker information includes generating target marker information associated with one or more of mRNA, RNA, DNA, peptide or protein.

6. The method of claim 1, wherein generating the target marker information includes generating target marker information associated with one or more of PLCXD1, HKR1, ZNF283, ADA, AMACR+p63, ANK3, BCL2L1, BIRCS, BMI-1, BUB1, CCNB1, CCND1, CES1, CHAF1A, CRIP1, CRYAB, ESM1, EZH2, FGFR2, FOS, Gbx2, HCFC1, IER3, ITPR1, JUNB, KLF6, KI67, KNTC2, MGC5466, Phc1, RNF2, Suz12, TCF2, TRAP100, USP22, Wnt5A and ZFP36.

7. The method of claim 1, wherein generating the aberrant object information includes generating aberrant sequence information when a quality of a sequence associated with the biological sample is distinct as compared with one or more reference sequences.

8. The method of claim 1, wherein generating the aberrant object information includes generating aberrant sequence information responsive to one or more inputs indicative of a distinct positioning of a plurality of bases within an entire sequence associated with the biological sample, as compared with one or more reference sequences.

9. The method of claim 1, wherein generating the aberrant object information includes generating aberrant sequence information responsive to one or more inputs indicative of a distinct fragment of a sequence associated with the biological sample, as compared with one or more reference sequences.

10. The method of claim 1, wherein generating the aberrant object information includes generating aberrant expression level information responsive to one or more inputs indicative of when an expression level exceeds a target threshold.

11. The method of claim 1, wherein generating the aberrant object information includes determining expression level aberrant score when a detected expression level is above a target threshold.

12. The method of claim 1, wherein generating the aberrant object information includes determining a sequence aberrant score when a detected positioning of a plurality of bases associated with the biological sample is distinct compared with a one or more reference sequences.

13. The method of claim 1, wherein generating the aberrant object information includes determining a sequence aberrant score responsive to one or more inputs from a next generation sequencing, multicolor quantitative immunofluorescence co-localization analysis, fluorescence in situ hybridization, and quantitative RT-PCR analysis.

14. The method of claim 1, wherein generating the aberrant object information includes determining a threshold level by comparing reference information derived from samples obtained from biological subjects with known diagnosis or known clinical outcome after therapies.

15. The method of claim 14, further comprising:
generating a cancer-therapy efficacy status, cancer therapy progress, a cancer prognosis, a cancer diagnosis responsive to one or more inputs indicative of an aberrant expression and an expression level above a target threshold coefficient of at least two markers.

16. The method of claim 1, wherein generating the aberrant object information includes generating aberrant sequence information and marker co-expression level information.

17. The method of claim 1, further comprising:
generating a cancer-therapy efficacy status responsive to one or more inputs indicative of an aberrant sequence and a threshold marker co-expression level.

18. The method of claim 1, further comprising:
generating information indicative of the presence or absence of cancer in a biological subject responsive to one or more inputs indicative of an aberrant sequence and a threshold marker co-expression level.

19. A system for diagnosing cancer or predicting cancer-therapy outcome in a subject, comprising:
circuitry configured to generate target marker information responsive to one or more inputs indicative of a genomic signature pathway and one or more inputs indicative of a proteomic signature pathway of endogenous human Stem Cell-Associated Retroviruses (SCAR); and
circuitry configured to generate aberrant object information responsive to comparing at least one input indicative of an expression levels and at least one input indicative of a sequence of a biological sample with target marker information.

20. The system of claim 19, further comprising:
circuitry configured to generate information indicative of the presence or absence of cancer in a biological subject responsive to one or more inputs indicative of an aberrant sequence and a threshold marker co-expression level.

21. The system of claim 19, further comprising:
circuitry configured to generate a cancer-therapy efficacy status, cancer therapy progress, a cancer prognosis, a cancer diagnosis responsive to one or more inputs indicative of an aberrant expression and an expression level above a target threshold coefficient of at least two markers.

22. The system of claim 19, further comprising:
circuitry configured to generate a cancer-therapy efficacy status responsive to one or more inputs indicative of an aberrant sequence and a threshold marker co-expression level.

23. A system for treating cancer, comprising:
circuitry configured to acquire information associated with a Stem Cell-Associated Retroviruses (SCAR) pathway activation in a subject diagnosed with cancer; and
circuitry configured to identify single therapeutic agent or combination of therapeutic agents and to generate user-specific treatment protocol responsive to one or more inputs associated with a Stem Cell-Associated Retroviruses (SCAR) pathway activation in a subject diagnosed with cancer.

24. A method for diagnosing cancer or predicting cancer-therapy outcome in a subject, comprising:
concurrently screening a biological sample for a presence of an aberrant sequence and an aberrant expression level of one or more target markers associated with a pathway involving genomic and proteomic signatures of endogenous human Stem Cell-Associated Retroviruses (SCAR);
scoring a sequence associated with the biological sample as aberrant when the quality of the sequence is distinct compared with a reference sequence; and
scoring an expression level associated with the biological sample as being aberrant when a detected expression level is above a target threshold coefficient.

25. The method of claim 24, wherein concurrently screening a biological sample for a presence of an aberrant sequence and an aberrant expression level of one or more target markers associated with a pathway involving genomic and proteomic signatures of endogenous SCAR includes concurrently screening a biological sample for a presence of an aberrant sequence.
and an aberrant expression level of one or more target markers indicative of a cancer diagnosis or a prognosis for cancer-therapy failure in a biological subject.

26. The method of claim 25, further comprising:
generating a user-specific cancer therapy protocol responsive to one or more inputs indicative of an aberrant sequence or an aberrant expression level associated with a cancer diagnosis or a prognosis for cancer-therapy failure in a biological subject.

27. The method of claim 24, wherein concurrently screening a biological sample for a presence of an aberrant sequence and an aberrant expression level of one or more target markers associated with a pathway involving genomic and proteomic signatures of endogenous SCAR includes concurrently screening a biological sample for a presence of an aberrant aberrant sequence and an aberrant expression level of one or more target markers indicative of a progress of cancer therapy in a biological subject.

28. The method of claim 27, further comprising:
generating a user-specific cancer therapy protocol responsive to one or more inputs indicative of an aberrant sequence or an aberrant expression level associated with a progress of cancer therapy in a biological subject.

29. The method of claim 24, wherein the detection threshold is being determined by comparing the values in a reference database of samples obtained from subjects with known diagnosis or known clinical outcome after therapies, wherein the presence of an aberrant expression level of at least one but preferably two or more markers in the test sample and presence of aberrant expression of two or more such markers is indicative of a cancer diagnosis or a prognosis for cancer-therapy failure, or of the progress of cancer therapy in the subject.

30. The method of claim 24, where the detection threshold is continuously refined by adding the outcome data of each patient tested to the reference database of samples, and in an automated and/or recursive manner either manually or using computational methods using data stored either locally, in remote server(s), or in the cloud, continuously improving the accuracy of diagnosis, prognosis, or specification of future cancer therapy.

31. The method of claim 24, wherein said sample phenotype is selected from the group consisting of cancer, non-cancer, recurrence, non-recurrence, relapse, non-relapse, invasiveness, non-invasiveness, metastatic, non-metastatic, localized, tumor size, tumor grade, Gleason score, survival prognosis, lymph node status, tumor stage, degree of differentiation, age, hormone receptor status, tumor antigen level (including but not limited to PSA level, PSMA level, survivin level, oncofetal protein level, testis antigen level), histologic type, level of, phenotype and genotype of and activation status of immune cells, and disease free survival.

32-40. (canceled)

41. A method of determining detection threshold for classifying a sample phenotype, comprising:
identifying a subset of markers and scoring marker expression in cells according to the method of claim 24; and
determining the sample classification accuracy at different detection thresholds using a reference database of samples from subjects with known phenotypes.

42. The method of claim 41, further comprising determining the sample classification accuracy in an automated and/or recursive manner either manually or using computational methods using data stored either locally, in remote server(s), or in the cloud.

43. The method of claim 41, further comprising determining the best performing magnitude of said detection threshold and using said magnitude to assess the reliability of said established detection threshold in classifying a sample phenotype.

44. The method of claim 41, further comprising determining the best performing magnitude of said detection threshold and using said magnitude to assess the reliability of said established detection threshold in classifying a sample phenotype in an automated and/or recursive manner either manually or using computational methods using data stored either locally, in remote server(s), or in the cloud.

45. The method of claim 41, further comprising using the best performing magnitude of said detection threshold to score an unclassified sample and assign a sample phenotype to said sample.

46. The method of claim 41, further comprising using the best performing magnitude of said detection threshold to score an unclassified sample and assign a sample phenotype to said sample either manually or using computational methods using data stored either locally, in remote server(s), or in the cloud.

47. The method of claim 41, wherein said subset of markers consists essentially of the genes, genetic loci, and sequences identified in Table 1A, Table 1, Table 2, Table 3, FIG. 16, FIGS. 18A and 18B, FIGS. 19A and 19B, FIGS. 20A-20C, FIGS. 21A-21C, Data Set S1, Data Set S2, or Data Set S3.

48-52. (canceled)

53. A method of treating cancer, comprising:
detecting a molecular signal(s) of SCAR's pathway activation in a subject diagnosed with cancer; and
generating a user-specific therapeutic treatment targeted to activated SCAR's loci and/or down-stream SCARs-regulated genetic loci based on detecting the molecular signal(s) of SCAR's pathway activation.

54. The method of claim 53, wherein the user-specific therapeutic treatment is based on genome editing to silence the defined genomic elements of the activated SCARs pathway.

55. The method of claim 53, wherein the user-specific therapeutic treatment is based on genome editing, including but not limited to CRISPR/Cas9 complex-mediated genome editing, to activate the defined genomic elements of the activated SCARs pathway.

56. The method of claim 53, wherein the user-specific therapeutic treatment is based on the application of Highly Active Anti-Retroviral Therapy (HAART).

57. The method of claim 53, wherein the user-specific therapeutic treatment is based on administration of the antiretroviral drug, Raltegravir (RAL, Isentress, formerly MK-0518).

58. The method of claim 53, wherein the user-specific therapeutic treatment is based on application of anti-sense therapy directed against transcriptionally active SCAR's loci and/or defined genomic elements of the activated SCARs pathway.

59. The method of claim 53, wherein the user-specific therapeutic treatment is based on the application of targeted immunotherapy, including at least one of antagonist antibodies or fragments thereof, agonist antibodies or fragments thereof, autologous cells, allogeneic cells, peptides, small molecules, signaling proteins or fragments thereof, or compositions containing two or more of the above and compositions containing in a single molecule or cellular therapy all or part of two or more of the above, directed against the proteins and/or peptides encoded by the activated SCARs sequences.

60. A method of treating cancer where the the method of claim 41 is used to enhance tumor infiltrating lymphocytes in tumors of treated subjects, either as a sole function or to augment the activity of anti-cancer modulators of the immune system.
